(19) United States
(12) Patent Application Publication
(10) Pub. No.: US 2026/0325653 A1
(43) Pub. Date: Oct. 22, 2026

(54) HETEROBIFUNCTIONAL SMALL MOLECULES THAT DEUBIQUITINYLATE AND STABILIZE TARGET PROTEINS BY RECRUITING USP7

(71) Applicants: ICAHN SCHOOL OF MEDICINE AT MOUNT SINAI, New York, NY (US); BETH ISTAEL DEACONESS MEDICAL CENTER, INC., Boston, MA (US)

(72) Inventors: Jian Jin, New York, NY (US); Wenyi Wei, Boston, MA (US); Yan Xiong, New York, NY (US); Jing Liu, Boston, MA (US); Kaixiu Luo, New York, NY (US); Xiaoping Hu, New York, NY (US); Zhen Wang, Boston, MA (US); Li Chen, Boston, MA (US)

(21) Appl. No.: 19/481,429

(22) PCT Filed: May 2, 2024
(86) PCT No.: PCT/US2024/027532

§ 371 Date: Nov. 4, 2025

Related U.S. Application Data
(60) Provisional application No. 63/500,505, filed on May 5, 2023.

(51) Int. Cl. A61K 47/55; A61K 47/54; A61P 11/00

(52) U.S. Cl. CPC A61K 47/55; A61K 47/545; A61P 11/00

(57) ABSTRACT

Disclosed is a method and compounds for treating AMPK, cGAS, or ΔF508-CFTR mediated diseases, the method including administering one or more AMPK, cGAS, or CFTR DUBTACs to a subject who has an AMPK, cGAS, or ΔF508-CFTR-mediated disease, the AMPK, cGAS, or CFTR DUBTACs being heterobifunctional small-molecule compounds (or bivalent compounds in short) including an AMPK, cGAS, or CFTR ligand conjugated to a de-ubiquitination tag (such as a small-molecule ligand of USP7) via a linker, which would stabilize AMPK, cGAS, or CFTR. The AMPK, cGAS, or ΔF508-CFTR-mediated disease can be a disease resulting from AMPK, cGAS, or CFTR destabilization. The AMPK, cGAS, or ΔF508-CFTR-mediated disease can have reduced AMPK, cGAS, or CFTR expression relative to a wild-type tissue of the same species and tissue type. The disclosed bivalent compounds stabilize AMPK, cGAS, or CFTR by recruiting USP7.

### CROSS-REFERENCE TO RELATED APPLICATION

The present application claims priority to U.S. Provisional Application Ser. No. 63/500,505, filed on May 5, 2023, the contents of which are hereby incorporated by reference in their entirety.

### STATEMENT REGARDING GOVERNMENT FUNDING

This invention was made with government support under R35CA253027 awarded by the National Institutes of Health. The government has certain rights in the invention.

### TECHNICAL FIELD

The present disclosure is directed to heterobifunctional small-molecule compounds that recruit USP7, a deubiquitinase, to deubiquitinylate and stabilize a target protein such as AMPK, cGAS and CFTR, and to methods of use of the heterobifunctional small-molecule compounds for the treatment of AMPK, cGAS, or CFTR-mediated diseases in a subject in need thereof. The disclosure also related to methods for identifying such heterobifunctional small-molecule compounds.

### BACKGROUND

Protein ubiquitination plays a critical role in a variety of cellular process, like protein degradation, quality control, trafficking, and signaling. Therefore, controlling the ubiquitination level of disease-relevant proteins can achieve substantial therapeutic effects. For example, inducing the ubiquitination of oncoproteins such as Myc and Ras to lead to their degradation by small molecules could become effective cancer therapy. Similarly, deubiquitination and stabilization of proteins such as tumor suppressors by small-molecule compounds can also become effective therapy for the treatment of relevant diseases including cancer.

Ubiquitin-specific-processing protease 7 (USP7), also known as herpesvirus-associated ubiquitin-specific protease (HAUSP), belongs to the deubiquitinase (DUB) family. USP7 is ubiquitously expressed in cells and has low tissue specificity. Therefore, USP7 is an excellent DUB to be recruited to achieve targeted protein stabilization (TPS).

5′ adenosine monophosphate-activated protein kinase (also known as AMPK) is an enzyme that plays key roles in cellular energy homeostasis. Recent studies have shown that AMPK regulates diverse metabolic and physiological processes. However, it is downregulated in major chronic diseases, such as obesity, inflammation, diabetes and cancer. Therefore, heterobifunctional small-molecule compounds that stabilize AMPK in cells and tissues, which leads to AMPK activation, can provide novel and effective therapeutics to treat such diseases (Jeon, 2016).

This TPS strategy can also be applied to other protein targets, such as cyclic GMP-AMP (cGAMP) synthase (also known as cGAS). The cGAS-stimulator of interferon genes (STING) pathway is a component of the innate immune system. cGAS detects the presence of cytosolic DNA and, in response, triggers expression of inflammatory genes that can lead to senescence or activation of defense mechanisms. In tumor cells, the cGAS-STING pathway exerts its antitumor effects through the induction of a robust type I IFN response, which activates immune cells, particularly dendritic cells (DCs), in the tumor micro environment (TME) (Samson and Ablasser, 2022).

Besides AMPK and cGAS, cystic fibrosis transmembrane conductance regulator (CFTR) is another suitable protein target for TPS. CFTR ΔF508 mutation destabilizes CFTR, and leads to the cystic fibrosis phenotype (Ward et al., 1995). Therefore, deubiquitination of ΔF508 mutant CFTR by heterobifunctional small-molecule compounds can be an effective strategy to prevent the cystic fibrosis process.

The targeted protein degradation (TPD) field has made great progress over the past 10 years. The PROteolysis TArgeting Chimera (PROTAC) technology has been widely utilized in drug discovery field (Dale et al., 2022). Deubiquitinase-targeting chimera (DUBTAC), which is a heterobifunctional small-molecule compound that hijacks a cellular deubiquitinase to remove the polyubiquitin attached to the target protein by inducing the close proximity between the target protein and deubiquitinase, recently emerged as an effective technology to improve target protein stability by reducing the proteasomal degradation of the target protein (Henning et al., 2022; Liu et al., 2022). To date, only OTUB1-recruiting DUBTACs have been reported (Henning et al., 2022; Liu et al., 2022). DUBTACs or heterobifunctional small-molecule compounds that recruit USP7 to deubiquitinylate and stabilize a target protein are unprecedented.

### SUMMARY

The ubiquitination and degradation of many proteins is the main cause of numerous diseases. Therefore, de-ubiquitination and stabilization of these disease-relevant proteins by DUBTACs or heterobifunctional small-molecule compounds can provide a novel therapeutic strategy.

In an aspect, this disclosure provides a method of treating AMPK, cGAS, or ΔF508-CFTR mediated diseases, the method including administering one or more AMPK, cGAS, or CFTR DUBTACs to a subject who has an AMPK, cGAS, or ΔF508-CFTR-mediated disease, the AMPK, cGAS, or CFTR DUBTACs being heterobifunctional small-molecule compounds (or bivalent compounds in short) including an AMPK, cGAS, or CFTR ligand conjugated to a de-ubiquitination tag (such as a small-molecule ligand of USP7) via a linker, which would stabilize AMPK, cGAS, or CFTR. The AMPK, cGAS, or ΔF508-CFTR-mediated disease can be a disease resulting from AMPK, cGAS, or CFTR destabilization. The AMPK, cGAS, or ΔF508-CFTR-mediated disease can have reduced AMPK, cGAS, or CFTR expression relative to a wild-type tissue of the same species and tissue type.

More specifically, the invention disclosed here provides bivalent compounds that stabilize AMPK, cGAS, or CFTR by recruiting USP7.

In some aspects, the AMPK, cGAS, or CFTR DUBTACs have the form “PI-Linker-USP7 Recruiter”, as shown below:

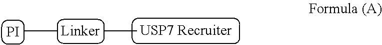

wherein PI (a ligand for a “protein of interest,” i.e., the protein to be de-ubiquitination) (e.g., a CFTR, AMPK or cGAS ligand), and USP7 Recruiter comprises a de-ubiquitination tag by binding with USP7. Exemplary CFTR, AMPK or cGAS ligands (PI), exemplary USP7 ligands (USP7 Recruiter), and exemplary linkers (Linker) are illustrated below:

### PI

### AMPK Ligand

In some embodiments, the AMPK ligand can be an AMPK activator or AMPK inhibitor which potently binds to AMPK. In some embodiments, the AMPK ligand comprises Metformin (Xiao et al., 2020), AICAR (Nakamaru et al., 2005), ZMP, AMP, A-769662 (Sanders et al., 2007), 991 (Ngoei et al., 2018), PF-06685249 (Edmonds et al., 2018), PXL770 (Cusi et al., 2021), MK-8722 (Myers et al., 2017), PF-739 (Aledavood et al., 2021), 4-azaindole, RSVA405 (Vingtdeux et al., 2011), COH-SR4 (Figarola and Rahbar, 2013), B10 (Sun et al., 2020), Dorsomorphin (Kim et al., 2011), BAY-3827 (Lemos et al., 2021), GSK-690693 (Rhodes et al., 2008) or a derivative thereof. In some embodiments, the AMPK ligand is a compound disclosed in one or more of WO2009124636, WO2009100130, WO2010036613, WO2011029855, WO2011080277, WO2011032320, WO2013116491, WO2014133008, WO2016008404, and WO2016001224, each of which is incorporated by reference in its entirety.

In some embodiments, the AMPK ligand is an AMPK inhibitor. In some embodiments, the AMPK ligand is an ATP-competitive AMPK inhibitor. In some embodiments, the AMPK ligand is an AMPK activator. In some embodiments, the AMPK ligand is an AMPK activator, where the AMPK activator binds to an allosteric binding site. In some embodiments, the AMPK activator binds to an allosteric binding site located at the α/β subunit interface. In some embodiments, the AMPK activator has different AMPK isoform selectivity. In some embodiments, the AMPK activator is a pan-AMPK isoform activator.

In some embodiments, the AMPK ligand comprise the structure of Formula (B-I):

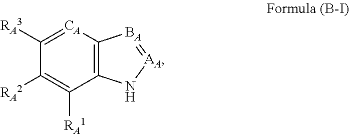

or a pharmaceutically acceptable salt of the compound or the tautomer thereof, wherein
A_{A} is selected from N, CR_{A}⁴, or

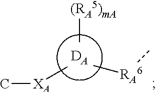

B_{A} is selected from N, CR_{A}⁴, or

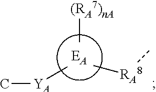

[symbol] denotes the point of attachment to Linker in Formula (A);
C_{A} is selected from N or CR_{A}⁴;
Ring D_{A} and Ring E_{A} are independently selected from null, C₃-C₁₂ cycloalkyl, 3-12-membered heterocyclic, C₆-C₁₀ aryl, and 5-10 membered heteroaryl;
X_{A} and Y_{A} are independently selected from a bond, —O—, —S—, —NR_{A}⁹—, —C(O)—, —C(O)O—, —C(O)NR_{A}⁹—, —O—C(O)NR_{A}⁹—, —NR_{A}¹⁰C(O)NR_{A}⁹—, —S(O)—, —S(O)₂—, —S(O)NR_{A}⁹—, —S(O)₂NR_{A}⁹—, C₁-C₆ alkylene, C₁-C₆ haloalkylene, C₁-C₆ heteroalkylene, C₂-C₆ alkenylene, and C₂-C₆ alkynylene;
R_{A}¹, R_{A}², each R_{A}⁴, each R_{A}⁵, and each R_{A}⁷ are independently selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic;
R_{A}³ is selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl; or
R_{A}¹ and R_{A}², R_{A}² and R_{A}³, R_{A}³ and R_{A}⁴, two R_{A}⁵, or two R_{A}⁷, together with the atoms to which they are attached, optionally form C₄-C₆ cycloalkyl or 5-7 membered heterocyclic, C₆-C₁₀ aryl, and 5-10 membered heteroaryl;
R_{A}⁶ and R_{A}⁸ are independently selected from a bond, —O—, —NR_{A}¹³—, —C(O)—, —C(O)O—, —C(O)NR_{A}¹³—, —O—C(O)NR_{A}¹³—, —NR_{A}¹⁴C(O)NR_{A}¹³—, —S(O)—, —S(O)₂—, —S(O)NR_{A}¹³—, —S(O)₂NR_{A}¹³—, C₁-C₆ alkylene, C₁-C₆ haloalkylene, C₁-C₆ heteroalkylene, C₂-C₆ alkenylene, C₂-C₆ alkynylene, C₆-C₁₀ arylene, and 5-10 membered heteroarylene;
each R_{A}⁹, each R_{A}¹⁰, each R_{A}¹¹, each R_{A}¹², each R_{A}¹³, and each R_{A}¹⁴ are independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl; or
R_{A}¹¹ and R_{A}¹² together with the atoms to which they are attached, optionally form 3-12 membered heterocyclic; and
m_{A} and n_{A} are independently selected from 0, 1, 2, 3, and 4.

In some embodiments, A_{A} is N; and B_{A} is

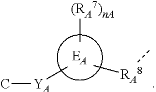

In some embodiments, A_{A} is CR_{A}⁴; and B_{A} is

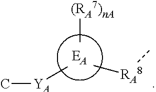

In some embodiments, A_{A} is

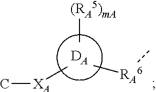

and B_{A} is N. In some embodiments, A_{A} is

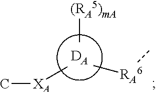

and B_{A} is CR_{A}⁴.

In some embodiments, the AMPK ligand comprise the structure of Formulae (B-I-a) and Formulae (B-I-b):

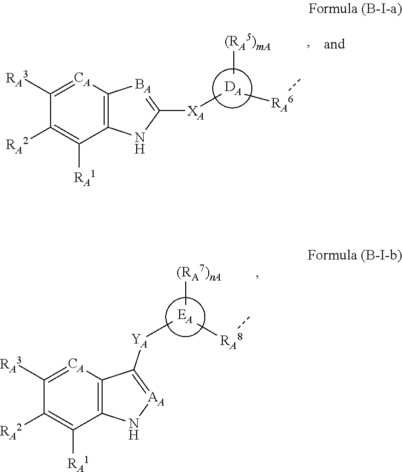

wherein
A_{A} and B_{A} are independently selected from N, CR_{A}⁴.

In some embodiments, X_{A} is selected from —O—, —S—, —NR_{A}⁹—, —C(O)—, C₁-C₆ alkylene, C₁-C₆ haloalkylene, C₁-C₆ heteroalkylene, C₆-C₁₀ arylene, and 5-10 membered heteroarylene. In some embodiments, X_{A} is selected from —O—, —S—, —NR_{A}⁹—, —C(O)—, methylene, and halomethylene. In some embodiments, X_{A} is selected from —O—, —S—, —NH—, —C(O)—, CH₂, and CF₂. In some embodiments, X_{A} is —O—.

In some embodiments, Y_{A} is selected from a bond, —C(O)—, —C(O)O—, —C(O)NR_{A}⁹—, —S(O)—, —S(O)₂—, —S(O)NR_{A}⁹—, —S(O)₂NR_{A}⁹—. In some embodiments, Y_{A} is selected from a bond, —C(O)—, —C(O)NR_{A}⁹—, and —S(O)₂NR_{A}⁹—. In some embodiments, Y_{A} is —C(O)NR_{A}⁹—.

In some embodiments, the AMPK ligand comprise the structure of Formulae (B-I-a1), (B-I-b1), and (B-I-b2):

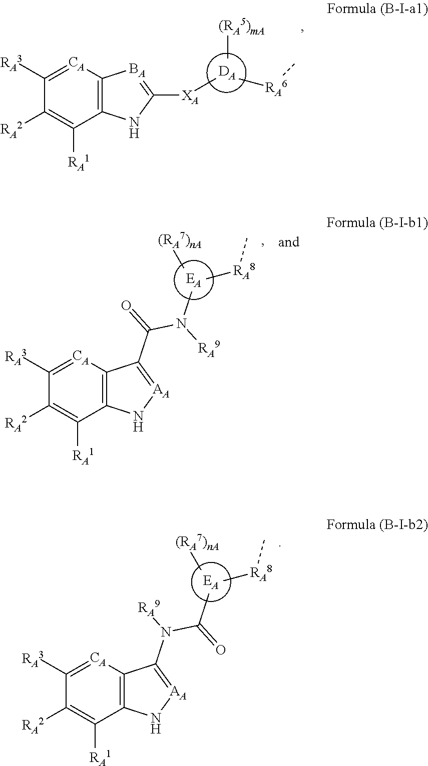

In some embodiments, A_{A} is N. In some embodiments, A_{A} is CR_{A}⁴. In some embodiments, B_{A} is N. In some embodiments, B_{A} is CR_{A}⁴. In some embodiments, C_{A} is N. In some embodiments, C_{A} is CR_{A}⁴.

In some embodiments, Ring D_{A} is selected from null, C₃-C₁₂ cycloalkyl, 3-12-membered heterocyclic, C₆-C₁₀ aryl, and 5-10 membered heteroaryl. In some embodiments, Ring D_{A} is C₃-C₁₂ cycloalkyl. In some embodiments, Ring D_{A} is selected from cyclobutyl, cyclopropyl, cyclobutyl, cyclopentyl, cyclohexyl, and cycloheptyl. In some embodiments, Ring D_{A} is 3-12-membered heterocyclic. In some embodiments, Ring D_{A} is selected from tetrahydrofuryl, hexahydrofuro[3,2-b]furyl, azetidinyl, pyrrolidinyl, piperidinyl, and piperazinyl. In some embodiments, Ring D_{A} is C₆-C₁₀ aryl. In some embodiments, Ring D_{A} is phenyl. In some embodiments, Ring D_{A} is 5-10 membered heteroaryl. In some embodiments, Ring D_{A} is selected from thiophenyl, benzothiophenyl, tetrahydrobenzothiophenyl, thiazolyl, imidazolyl, furanyl, pyrrolyl, pyrazolyl, pyridinyl, pyrimidinyl, pyridazinyl, pyrazinyl, benzofuranyl, indolyl, and indazolyl.

In some embodiments, Ring E_{A} selected from null, C₃-C₁₂ cycloalkyl, 3-12-membered heterocyclic, C₆-C₁₀ aryl, and 5-10 membered heteroaryl. In some embodiments, Ring E_{A} selected from null, C₆-C₁₀ aryl, and 5-10 membered heteroaryl. In some embodiments, Ring E_{A} selected from null. In some embodiments, Ring E_{A} is C₆-C₁₀ aryl. In some embodiments, Ring E_{A} is phenyl. In some embodiments, Ring E_{A} is 5-10 membered heteroaryl. In some embodiments, Ring E_{A} is selected from thiophenyl, benzothiophenyl, tetrahydrobenzothiophenyl, thiazolyl, imidazolyl, furanyl, pyrrolyl, pyrazolyl, pyridinyl, pyrimidinyl, pyridazinyl, pyrazinyl, benzofuranyl, indolyl, and indazolyl.

In some embodiments, the AMPK ligand comprise the structure of Formulae (B-I-a2), (B-I-a3), (B-I-a4), (B-I-b3), and (B-I-b4):

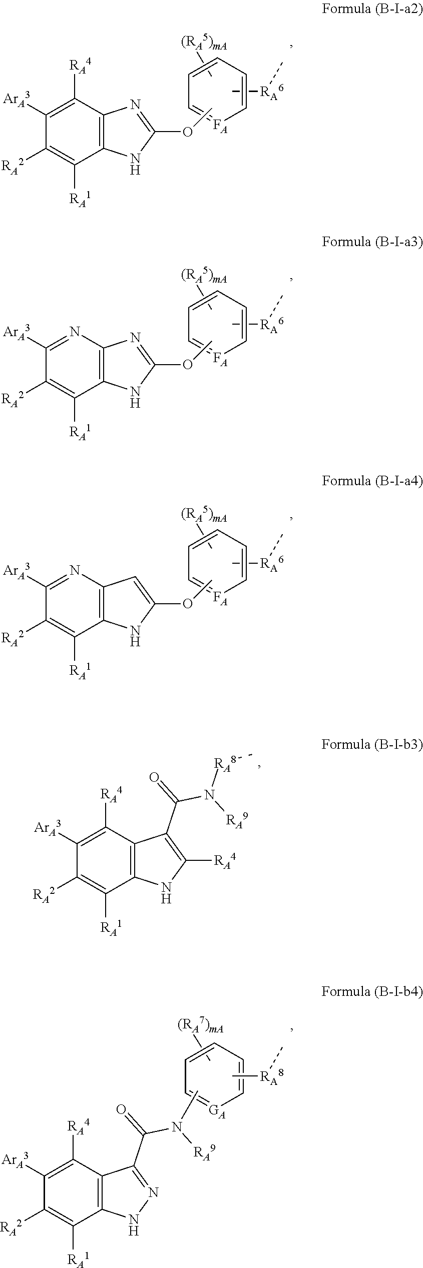

wherein
F_{A} is selected from N or CR_{A}⁵; and
G_{A} is selected from N or CR_{A}⁷.

In some embodiments, R_{A}³ is selected from C₃-C₁₂ cycloalkyl, 3-12 membered heterocyclic, C₆-C₁₀ aryl, and 5-10 membered heteroaryl. In some embodiments, R_{A}³ is selected from C₆-C₁₀ aryl and 5-10 membered heteroaryl.

In some embodiments, the AMPK ligand comprise the structure of Formulae (B-I-a5), (B-I-a6), (B-I-a7), (B-I-b5), and (B-I-b6):

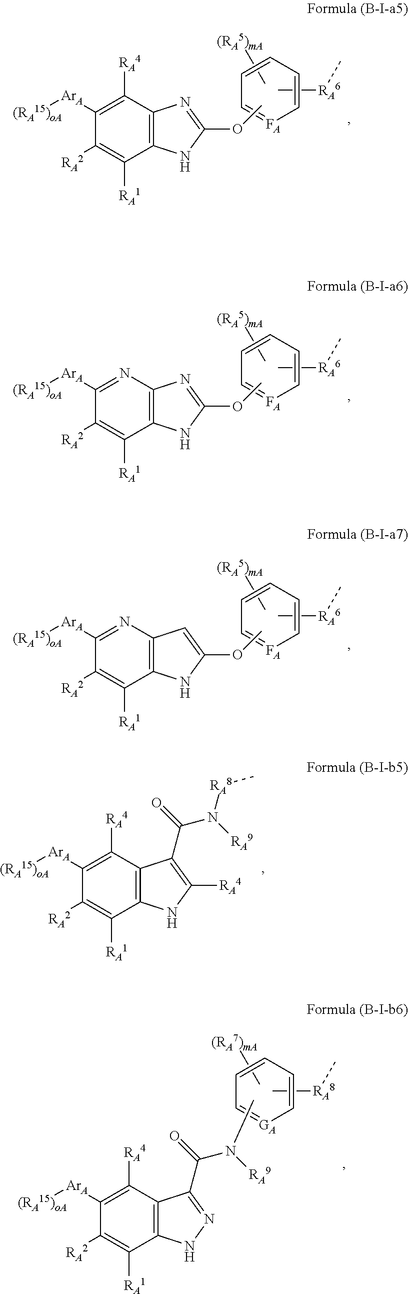

wherein
Ar_{A} is selected from C₆-C₁₀ aryl, or 5-10 membered heteroaryl;
each R_{A}¹⁵ is independently selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹¹R_{A}¹², —N═S(O)R_{A}¹¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl and 5-10 membered heteroaryl, or
two of R_{A}¹⁵, together with the atoms to which they are attached, optionally form C₃-C₁₂ cycloalkyl, 3-12 membered heterocyclic, C₆-C₁₀ aryl or 5-10 membered heteroaryl; and
o_{A} is selected from 0, 1, 2, 3, 4, 5, or 6.

In some embodiments, Ar_{A} is selected from C₆-C₁₀ aryl and 5-10 membered heteroaryl. In some embodiments, Ar_{A} is selected from phenyl, naphthalene, indane, 5,6,7,8-tetrahydronaphthalene, biphenyl, pyrazole, pyridine, pyrazine, pyrimidine, thiazole, thiophene, benzoimidazole, quinoline, isoquinoline, indole, indazole, carbazole, benzotriazole, benzofuran, benzothiazole, benzo[b]thiophene, benzo[d]isooxazole, 3,4-dihydro-2H-benzo[1,4]oxazine, benzo[1,3]dioxole, benzo[1,4]dioxane, 1H-pyrrolo[2,3-b]pyridine, [1,2,4]triazolo[4,3-a]pyridine, 3,4 dihydropyrido[3,2-b][1,4]oxazine, 3,4-dihydro-2H-1,4-benzoxazine, 2,3-dihydro-1H-indole, 2,3-dihydro-1H-isoindole, 2,3-dihydrobenzoimidazole, 1,2-dihydroquinoline, 1,2,3,4-tetrahydroisoqumoline, 1,2,3,4-tetrahydrocyclopenta[b]indole, 1,2,3,4-tetrahydroquinoxaHne, 1,2,3,6-tetrahydropyridine. In some embodiments, Ar_{A} is selected from phenyl, pyridine, biphenyl, and indole. In some embodiments, Ar_{A} is indole.

In some embodiments, R_{A}⁶ is selected from a bond, —O—, —NR_{A}¹³—, —C(O)—, —C(O)O—, —C(O)NR_{A}¹³—, —S(O)₂NR_{A}¹³—, C₁-C₆ alkylene, C₁-C₆ haloalkylene, and C₁-C₆ heteroalkylene. In some embodiments, R_{A}⁶ is selected from —C(O)—, —C(O)O—, and —C(O)NR_{A}¹³—. In some embodiments, R_{A}⁶ is —C(O)NR_{A}¹³—In some embodiments, R_{A}⁶ is

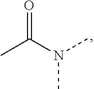

where the N atom of the amide attaches two Linker-USP7 Recruiter moieties.

In some embodiments, R_{A}⁸ is selected from a bond, —O—, —NR_{A}¹³—, —C(O)—, —C(O)O—, —C(O)NR_{A}¹³—, —S(O)₂NR_{A}¹³—, C₁-C₆ alkylene, C₁-C₆ haloalkylene, and C₁-C₆ heteroalkylene.

In some embodiments, R_{A}¹ is selected from H, halogen, cyano, C₁-C₆ alkyl, C₁-C₆ haloalkyl, C₁-C₆ heteroalkyl, C₃-C₁₂ cycloalkyl, and 3-12 membered heterocyclic. In some embodiments, R_{A}¹ is selected from H and CH₃. In some embodiments, R_{A}¹ is H. In some embodiments, R_{A}¹ is CH₃.

In some embodiments, R_{A}² is selected from H, halogen, cyano, C₁-C₆ alkyl, C₁-C₆ haloalkyl, C₁-C₆ heteroalkyl, C₃-C₁₂ cycloalkyl, and 3-12 membered heterocyclic. In some embodiments, R_{A}² is selected from halogen. In some embodiments, R_{A}² selected from F or C₁. In some embodiments, R_{A}² is F. In some embodiments, R_{A}² is Cl.

In some embodiments, each R_{A}⁴ is selected from H, halogen, cyano, C₁-C₆ alkyl, C₁-C₆ haloalkyl, C₁-C₆ heteroalkyl, C₃-C₁₂ cycloalkyl, and 3-12 membered heterocyclic. In some embodiments, R_{A}⁴ is H.

In some embodiments, each R_{A}⁵ is selected from H, halogen, cyano, C₁-C₆ alkyl, C₁-C₆ haloalkyl, C₁-C₆ heteroalkyl, C₃-C₁₂ cycloalkyl, and 3-12 membered heterocyclic. In some embodiments, each R_{A}⁵ is selected from H, halogen, C₁-C₆ alkyl, C₁-C₆ haloalkyl, C₁-C₆ heteroalkyl, C₃-C₁₂ cycloalkyl. In some embodiments, R_{A}⁵ is H, CH₃, CH₂CH₃, and CH₂OH.

In some embodiments, each R_{A}⁷ is selected from H, halogen, cyano, C₁-C₆ alkyl, C₁-C₆ haloalkyl, C₁-C₆ heteroalkyl, C₃-C₁₂ cycloalkyl, and 3-12 membered heterocyclic. In some embodiments, each R_{A}⁷ is selected from H, halogen, C₁-C₆ alkyl, C₁-C₆ haloalkyl, C₁-C₆ heteroalkyl, C₃-C₁₂ cycloalkyl. In some embodiments, R_{A}⁷ is H, CH₃, and CH₂CH₃.

In some embodiments, each R_{A}⁹ is independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl. In some embodiments, each R_{A}⁹ is independently selected from H, C₁-C₆ alkyl, C₁-C₆ haloalkyl, C₁-C₆ heteroalkyl, C₃-C₁₂ cycloalkyl, and 3-12 membered heterocyclic. In some embodiments, R_{A}⁹ is H.

In some embodiments, each R_{A}¹⁰ is independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl. In some embodiments, each R_{A}¹⁰ is independently selected from H, C₁-C₆ alkyl, C₁-C₆ haloalkyl, C₁-C₆ heteroalkyl, C₃-C₁₂ cycloalkyl, and 3-12 membered heterocyclic. In some embodiments, R_{A}¹⁰ is H.

In some embodiments, each R_{A}¹¹ and each R_{A}¹² are independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl. In some embodiments, each R_{A}¹¹ and each R_{A}¹² are independently selected from H, C₁-C₆ alkyl, C₁-C₆ haloalkyl, C₁-C₆ heteroalkyl, C₃-C₁₂ cycloalkyl, and 3-12 membered heterocyclic. In some embodiments, R_{A}¹¹ is selected from H and CH₃. In some embodiments, R_{A}¹² is selected from H and CH₃.

In some embodiments, R_{A}¹¹ and R_{A}¹² together with the atoms to which they are attached, optionally form 3-12 membered heterocyclic.

In some embodiments, each R_{A}¹³ is independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl. In some embodiments, each R_{A}¹³ is independently selected from H, C₁-C₆ alkyl, C₁-C₆ haloalkyl, C₁-C₆ heteroalkyl, C₃-C₁₂ cycloalkyl, and 3-12 membered heterocyclic.

In some embodiments, each R_{A}¹⁴ is independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl. In some embodiments, each R_{A}¹⁴ is independently selected from H, C₁-C₆ alkyl, C₁-C₆ haloalkyl, C₁-C₆ heteroalkyl, C₃-C₁₂ cycloalkyl, and 3-12 membered heterocyclic.

In some embodiments, each R_{A}¹⁵ is independently selected from each R_{A}¹⁵ is independently selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl and 5-10 membered heteroaryl.

In some embodiments, each R_{A}¹⁵ is independently selected from each R_{A}¹⁵ is independently selected from H, halogen, cyano, OR_{A}¹¹, NR_{A}¹¹R_{A}¹², S(O)₂NR_{A}¹¹R_{A}¹², —N═S(O)R_{A}¹¹R_{A}¹², C₁-C₆ alkyl, C₁-C₆ haloalkyl, C₁-C₆ heteroalkyl, C₃-C₁₂ cycloalkyl, 3-12 membered heterocyclic, C₆-C₁₀ aryl and 5-10 membered heteroaryl. In some embodiments, each R_{A}¹⁵ is independently selected from each R_{A}¹⁵ is independently selected from H, CN, CH₃, cyclopropyl, hydroxymethyl cyclopropyl, OCH₃, dimethyl amino, —N═S(O)(CH₃)₂, and -Ph-N═S(O)(CH₃)₂.

In some embodiments, m_{A} is selected from 0, 1, 2, 3, and 4. In some embodiments, m_{A} is selected from 0, 1, and 2. In some embodiments, n_{A} is selected from 0, 1, 2, 3, and 4. In some embodiments, n_{A} is selected from 0, 1, and 2. In some embodiments, o_{A} is selected from 0, 1, 2, 3, 4, and 5.

In some embodiments, the AMPK ligand comprise the structure of Formulae (B-I), (B-I-a), (B-I-a1), (B-I-a2) and (B-I-a5).

In some embodiments, the AMPK ligand comprise a derivative of following compounds:

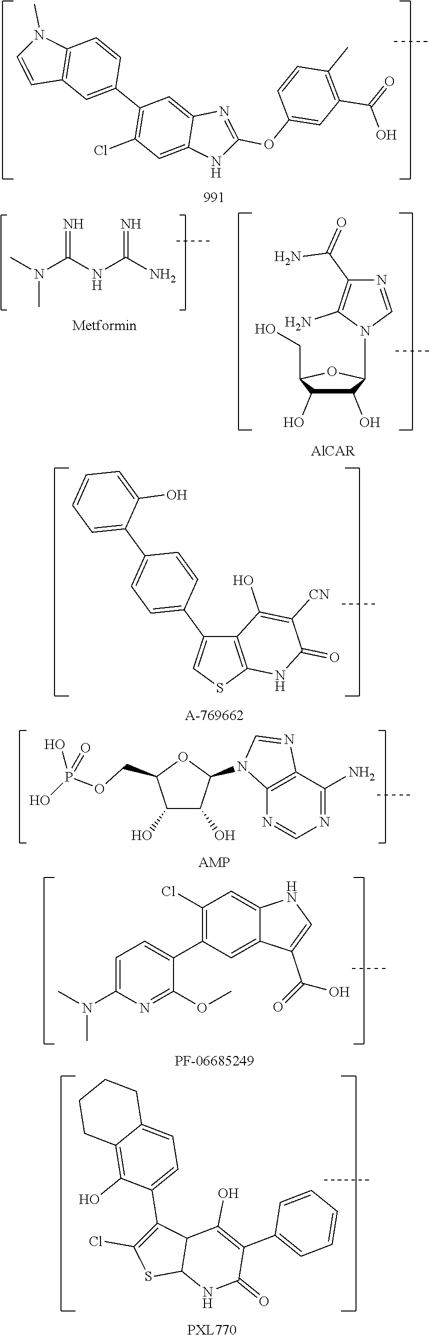

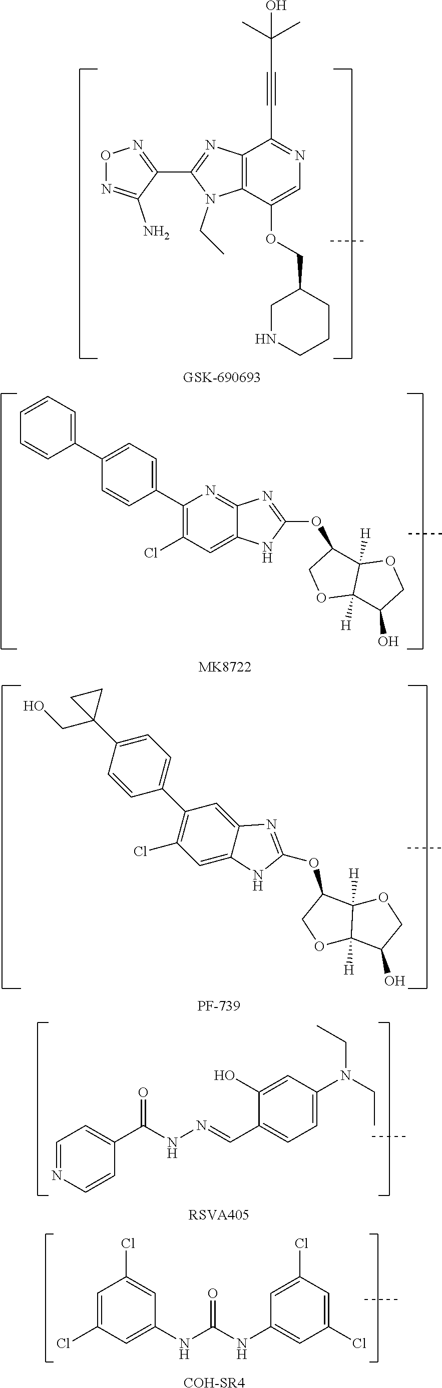

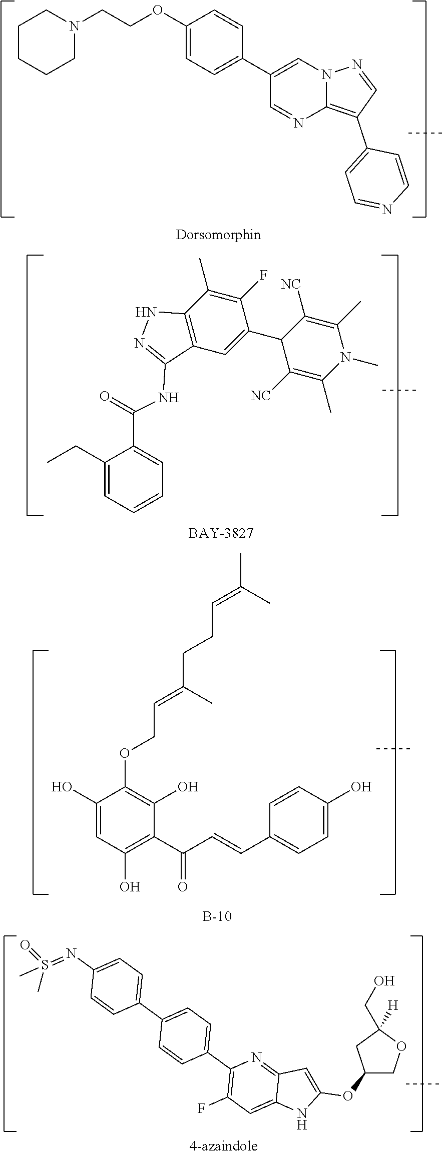

Wherein, [symbol] denotes the point of attachment to Linker.

One skilled in the art recognizes that there are sites on the ligand and recruiter that are not necessary for binding of the ligand and recruiter to the target protein and USP7, respectively, and would be appropriate for attaching a linker, provided that the linker, once attached to the ligand and recruiter does not significantly interfere with the binding of the bivalent compound to the target protein and USP7. Once a site is selected, one skilled in the art is able to chemically modify the ligand and/or recruiter to provide a derivative compound having a free valence at the desired site for attachment to a linker.

cGAS Ligand

In some embodiments, the cGAS ligand is selected from a cGAS activator or cGAS inhibitor which potently binds with cGAS. In some embodiments, the cGAS ligand is cGAS activator. In some embodiments, the cGAS ligand is cGAS inhibitor. In some embodiments, the cGAS ligand comprises hydroxychloroquinine (HCQ), Quinacrine (QC), X6, RU114757, RU191752, RU100840, RU.365, RU.521, J001, G001, G108, G150, G140, CPD-25, asprin, CPD-C, PF-06928215 (Zhao et al., 2022). In some embodiments, the cGAS ligand is a compound disclosed in one or more of U.S. 62/318,435, U.S. 62/355,403, U.S. 2018/0230115, WO2019241787, WO2019153002, WO2022140387, WO2023004437, each of which is incorporated herein by reference in its entirety.

In some embodiments, the cGAS ligand comprise the structure of Formula (C—I):

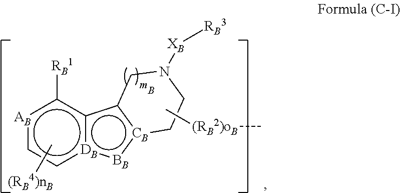

or a pharmaceutically acceptable salt of the compound or the tautomer thereof,
wherein
[symbol] denotes the point of attachment to Linker in Formula (A);
[symbol] connects to either R_{B}^{1b} or R_{B}^{3b}, with the proviso that when [symbol] connects to R_{B}^{1b}, R_{B}³ is R_{B}^{3a}; and when [symbol] connects to R_{B}^{3b}, R_{B}¹ is R_{B}^{1a};
A_{B} is selected from N and CR_{B}⁴;
B_{B} is selected from N, O, S, CR_{B}⁵ and NR_{B}⁵;
C_{B} is selected from N and C;
D_{B} is selected from N and C;
X_{B} is X_{B}^{a}—X_{B}^{b}.
X_{B}^{a} is selected from a bond, —C(O)—, —C(O)O—, —C(O)NR_{B}⁶—, —S(O)—, —S(O)₂—, —S(O)NR_{B}⁶—, —S(O)₂NR_{B}⁶—;
X_{B}^{b} is selected from a bond, C₁-C₆ alkylene, C₁-C₆ haloalkylene, C₁-C₆ heteroalkylene, C₃-C₁₂ cycloalkylene, and 3-12 membered heterocyclicene, C₆-C₁₀ arylene, and 5-10 membered heteroarylene;
R_{B}¹ is selected from monovalent group R_{B}^{1a} and bivalent group R_{B}^{1b};
R_{B}^{1a} is selected from H, halogen, cyano, OR_{B}⁷, NR_{B}⁷R_{B}⁸, C(O)OR_{B}⁷, C(O)NR_{B}⁷R_{B}⁸, S(O)₂NR_{B}⁷R_{B}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl;
R_{B}^{1b} is connected to the Linker, and is selected from a bond, —O—, —NR_{B}⁷—, —C(O)O—, —C(O)NR_{B}⁷—, —S(O)₂NR_{B}⁷—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, 3-12 membered heterocyclicene, C₆-C₁₀ arylene, and 5-10 membered heteroarylene;
each R_{B}² is independently selected from H, halogen, C₁-C₆ alkyl, C₁-C₆ haloalkyl, C₁-C₆ heteroalkyl, C₃-C₁₂ cycloalkyl, or
two R_{B}², together with the atoms to which they are attached, optionally form C₃-C₁₂ cycloalkyl or 3-12 membered heterocyclic;
R_{B}³ is selected from monovalent group R_{B}^{3a} and bivalent group R_{B}^{3b};
R_{B}^{3a} is selected from H, halogen, OR_{B}⁹, NR_{B}⁹R_{B}¹⁰, C(O)OR_{B}⁹, C(O)NR_{B}⁹R_{B}¹⁰, S(O)₂NR_{B}⁹R_{B}¹⁰, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic;
R_{B}^{3b} is connected to the Linker, and is selected from a bond, —O—, —NR_{B}⁹—, —C(O)O—, —C(O)NR_{B}⁹—, —S(O)₂NR_{B}⁹—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, and 3-12 membered heterocyclicene;
each R_{B}⁴ is independently selected from H, halogen, cyano, OR_{B}⁷, NR_{B}⁷R_{B}⁸, C(O)OR_{B}⁷, C(O)NR_{B}⁷R_{B}⁸, S(O)₂NR_{B}⁷R_{B}⁸, C₁-C₃ alkyl, C₁-C₃ haloalkyl, C₁-C₃ heteroalkyl, C₂-C₄ alkenyl, C₂-C₄ alkynyl, C₃-C₆ cycloalkyl, and 3-6 membered heterocyclic, or
two R_{B}⁴, together with the atoms to which they are attached, optionally form partially unsaturated C₃-C₁₂ cycloalkyl, partially unsaturated 3-12 membered heterocyclic, C₆-C₁₀ arylene, or 5-10 membered heteroarylene;
R_{B}⁵ is selected from H, halogen, C₁-C₃ alkyl, C₁-C₃ haloalkyl, C₁-C₃ heteroalkyl, C₃-C₆ cycloalkyl, and 3-6 membered heterocyclic;
R_{B}⁶, each R_{B}⁷, each R_{B}⁸, each R_{B}⁹, and each R_{B}¹⁰, are independently selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic, or
R_{B}⁷ and R_{B}⁸, and R_{B}⁹ and R_{B}¹⁰ together with the atoms to which they are attached, optionally form 3-12 membered heterocyclic;
m_{B} is selected from 1 and 2;
n_{B} is selected from 0, 1, 2 and 3; and
o_{B} is selected from 0, 1, 2, 3, 4, 5 and 6.

In some embodiments, [symbol] connects to R_{B}^{1b}, R_{B}³ is R_{B}^{3a}. In some embodiments, [symbol] connects to R_{B}^{3b}, R_{B}¹ is R_{B}^{1a}.

In some embodiments, the cGAS ligand comprise the structure of Formulae (C—I-a) and (C—I-b):

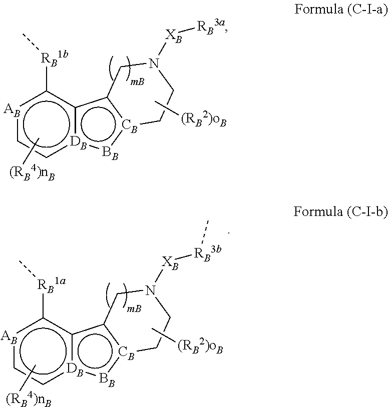

In some embodiments, A_{B} is CR_{B}⁴. In some embodiments, B_{B} is NR_{B}⁵. In some embodiments, C_{B} is C. In some embodiments, D_{B} is C.

In some embodiments, the cGAS ligand comprise the structure of Formulae (C—I-a1) and (C—I-b1):

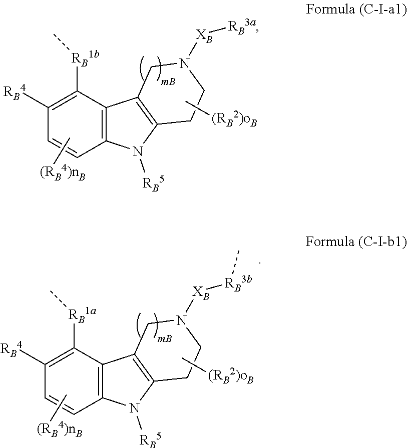

In some embodiments, R_{B}^{1a} is selected from H, halogen, C₃-C₁₂ cycloalkyl, 3-12 membered heterocyclic, C₆-C₁₀ aryl, and 5-10 membered heteroaryl. In some embodiments, R_{B}^{1a} is selected from C₃-C₁₂ cycloalkyl, 3-12 membered heterocyclic, C₆-C₁₀ aryl, and 5-10 membered heteroaryl. In some embodiments, R_{B}^{1a} is selected from C₆-C₁₀ aryl, and 5-10 membered heteroaryl. In some embodiments, R_{B}^{1a} is 5-10 membered heteroaryl. In some embodiments, R_{B}^{1a} is selected from furyl, thiophenyl, pyrrolyl, pyrazolyl, oxazolyl, oxadiazolyl, thiazolyl, isoxazolyl, isothiazolyl, imidazolyl, triazolyl, pyridinyl, pyrimidinyl, pyrazinyl, and pyridazinyl. In some embodiments, R_{B}^{1a} is pyrazolyl. In some embodiments, R_{B}^{1a} is =pyridinyl.

In some embodiments, R_{B}^{1b} is selected from C₃-C₁₂ cycloalkylene, 3-12 membered heterocyclicene, C₆-C₁₀ arylene, and 5-10 membered heteroarylene. In some embodiments, R_{B}^{1b} is selected from C₆-C₁₀ arylene, and 5-10 membered heteroarylene.

In some embodiments, the cGAS ligand comprise the structure of Formulae (C—I-a2) and (C—I-b2):

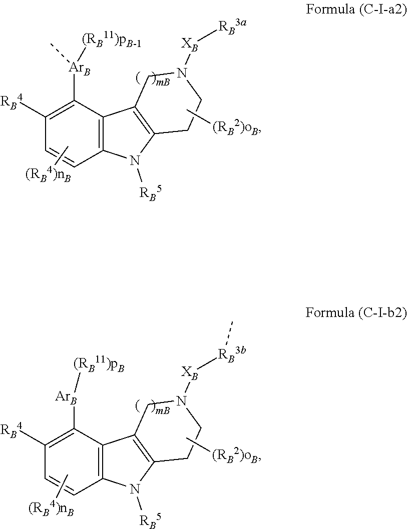

wherein
Ar_{B} is selected from C₆-C₁₀ aryl, and 5-10 membered heteroaryl;
each R_{B}¹¹ is independently selected from H, halogen, cyano, OR_{B}⁷, NR_{B}⁷R_{B}⁸, C(O)OR_{B}⁷, C(O)NR_{B}⁷R_{B}⁸, S(O)₂NR_{B}⁷R_{B}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl; or
two R_{B}¹¹, together with the atoms to which they are attached, optionally form partially unsaturated C₃-C₁₂ cycloalkyl, partially unsaturated 3-12 membered heterocyclic, C₆-C₁₀ aryl, or 5-10 membered heteroaryl;
p_{B} is selected from 0, 1, 2, 3, 4, and 5; and
p_{B-1} is selected from 0, 1, 2, 3, and 4.

In some embodiments, X_{B}^{a} is selected from —C(O)—, —S(O)—, and —S(O)₂—. In some embodiments, X_{B}^{a} is —C(O)—. In some embodiments, X_{B}^{b} is selected from C₁-C₆ alkylene, C₁-C₆ haloalkylene, C₁-C₆ heteroalkylene, C₃-C₁₂ cycloalkylene, and 3-12 membered heterocyclicene.

In some embodiments, the cGAS ligand comprise the structure of Formulae (C—I-a3) and (C—I-b3):

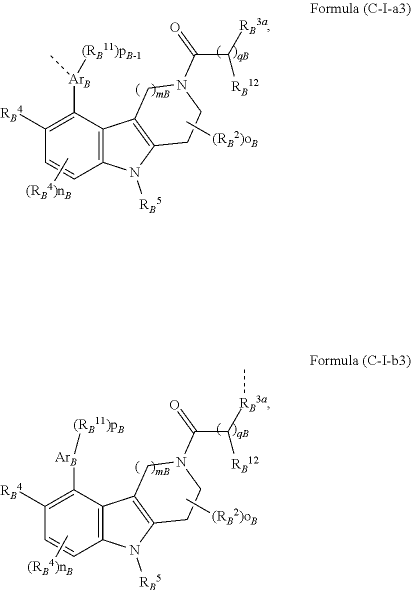

wherein
each R_{B}¹² is independently selected from H, halogen, OR_{B}⁷, NR_{B}⁷R_{B}⁸, C₁-C₆ alkyl, C₁-C₆ haloalkyl, C₁-C₆ heteroalkyl, C₃-C₁₂ cycloalkyl, and 3-12 membered heterocyclic; or
two R_{B}¹², R_{B}¹² and R_{B}^{3a}, and R_{B}¹² and R_{B}^{3b}, together with the atoms to which they are attached, optionally form C₃-C₁₂ cycloalkyl, 3-12 membered heterocyclic; and
q_{B} is selected from 0, 1, 2, 3, 4, and 5.

In some embodiments, Ar_{B} is 5-10 membered heteroaryl. In some embodiments, Ar_{B} is selected from furyl, thiophenyl, pyrrolyl, pyrazolyl, oxazolyl, oxadiazolyl, thiazolyl, isoxazolyl, isothiazolyl, imidazolyl, triazolyl, pyridinyl, pyrimidinyl, pyrazinyl, and pyridazinyl. In some embodiments, Ar_{B} is pyrazolyl. In some embodiments, Ar_{B} is pyridinyl.

In some embodiments, the cGAS ligand comprise the structure of Formulae (C—I-a4) and (C—I-b4):

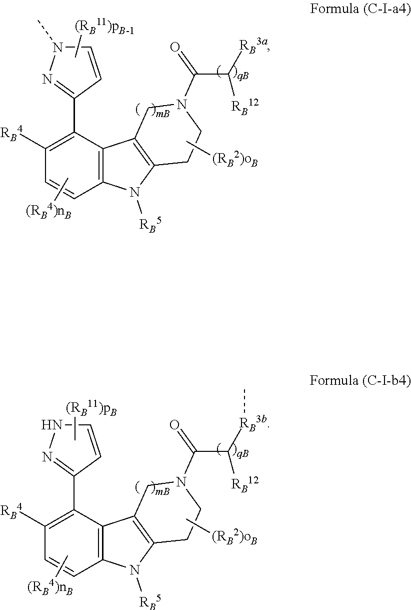

In some embodiments, each R_{B}² is independently selected from H, C₁-C₃ alkyl, C₁-C₃ haloalkyl, C₁-C₃ heteroalkyl, C₃-C₆ cycloalkyl. In some embodiments, R_{B}² is H. In some embodiments, R_{B}² is C₁-C₃ alkyl. In some embodiments, R_{B}² is CH₃. In some embodiments, two R_{B}², together with the atoms to which they are attached, optionally form C₃-C₆ cycloalkyl or 3-6 membered heterocyclic. In some embodiments, two R_{B}², together with the atoms to which they are attached, optionally form C₃-C₅ cycloalkyl.

In some embodiments, R_{B}^{3a} is selected from H, halogen, OR_{B}⁹, NR_{B}⁹R_{B}¹⁰, C₁-C₆ alkyl, C₁-C₆ haloalkyl, C₁-C₆ heteroalkyl, C₃-C₁₂ cycloalkyl, and 3-12 membered heterocyclic. In some embodiments, R_{B}^{3a} is selected from H, OR_{B}⁹ and NR_{B}⁹R_{B}¹⁰. In some embodiments, R_{B}^{3a} is selected from H, OH, OCH₃, OCH₂CH₂OCH₃, NH₂, NHCH₃, and NHC(O)CH₂NH₂. In some embodiments, R_{B}^{3a} is OH.

In some embodiments, R_{B}^{3b} is selected from a bond, —O—, —NR_{B}⁹—, C₁-C₆ alkylene, C₁-C₆ haloalkylene, C₁-C₆ heteroalkylene, C₃-C₁₂ cycloalkylene, and 3-12 membered heterocyclicene. In some embodiments, R_{B}^{3b} is selected from a bond, —O—, —NR_{B}⁹—. In some embodiments, R_{B}^{3b} is selected from a bond, —O—, —NH—, and —N(CH₃)—. In some embodiments, R_{B}^{3b} is —O—.

In some embodiments, each R_{B}⁴ is independently selected from H, halogen, cyano, OR_{B}⁷, NR_{B}⁷R_{B}⁸, C₁-C₃ alkyl, C₁-C₃ haloalkyl, C₁-C₃ heteroalkyl, C₂-C₄ alkenyl, C₂-C₄ alkynyl, C₃-C₆ cycloalkyl, and 3-6 membered heterocyclic. In some embodiments, each R_{B}⁴ is independently selected from H. In some embodiments, each R_{B}⁴ is independently selected from cyano. In some embodiments, each R_{B}⁴ is independently selected from halogen. In some embodiments, each R_{B}⁴ is independently selected from F. In some embodiments, each R_{B}⁴ is independently selected from Cl. In some embodiments, each R_{B}⁴ is independently selected from methyl, ethyl, propyl, isopropyl, cyclopropyl, trifluoromethyl, difluoromethyl, methoxy, ethoxy, cyclopropoxy, and azetidinyl. In some embodiments, each R_{B}⁴ is independently selected from H. In some embodiments, each R_{B}⁴ is independently selected from cyano. In some embodiments, each R_{B}⁴ is independently selected from halogen. In some embodiments, each R_{B}⁴ is independently selected from F. In some embodiments, each R_{B}⁴ is independently selected from Cl. In some embodiments, each R_{B}⁴ is independently selected from Br.

In some embodiments, two R_{B}⁴, together with the atoms to which they are attached, optionally form partially unsaturated C₃-C₆ cycloalkyl, partially unsaturated 3-6 membered heterocyclic, C₆-C₁₀ aryl, or 5-10 membered heteroaryl.

In some embodiments, R_{B}⁵ is selected from H, C₁-C₃ alkyl, C₁-C₃ haloalkyl, C₁-C₃ heteroalkyl, C₃-C₆ cycloalkyl, and 3-6 membered heterocyclic. In some embodiments, R_{B}⁵ is selected from H and C₁-C₃ alkyl. In some embodiments, R_{B}⁵ is H. In some embodiments, R_{B}⁵ is CH₃.

In some embodiments, R_{B}⁶, each R_{B}⁷, each R_{B}⁸, each R_{B}⁹, and each R_{B}¹⁰, are independently selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic. In some embodiments, R_{B}⁶, each R_{B}⁷, each R_{B}⁸, each R_{B}⁹, and each R_{B}¹⁰, are independently selected from H, methyl, ethyl, propyl, isopropyl, cyclopropyl, trifluoromethyl, and difluoromethyl. In some embodiments, R_{B}⁶, each R_{B}⁷, each R_{B}⁸, each R_{B}⁹, and each R_{B}¹⁰, are H. In some embodiments, R_{B}⁶, each R_{B}⁷, each R_{B}⁸, each R_{B}⁹, and each R_{B}¹⁰, are methyl.

In some embodiments, R_{B}⁷ and R_{B}⁸, and R_{B}⁹ and R_{B}¹⁰ together with the atoms to which they are attached, optionally form 3-6 membered heterocyclic.

In some embodiments, each R_{B}¹¹ is independently selected from H, halogen, cyano, OR_{B}⁷, NR_{B}⁷R_{B}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic. In some embodiments, R_{B}¹¹ is H. In some embodiments, each R_{B}¹¹ is CH₃.

In some embodiments, two R_{B}¹¹, together with the atoms to which they are attached, optionally form partially unsaturated C₃-C₁₂ cycloalkyl, 3-12 membered partially unsaturated heterocyclic, C₆-C₁₀ aryl, or 5-10 membered heteroaryl.

In some embodiments, each R_{B}¹² is independently selected from H, C₁-C₆ alkyl, C₁-C₆ haloalkyl, C₁-C₆ heteroalkyl, C₃-C₁₂ cycloalkyl, and 3-12 membered heterocyclic. In some embodiments, each R_{B}¹² is independently selected from H, and C₁-C₆ alkyl. In some embodiments, R_{B}¹² is H.

In some embodiments, two R_{B}¹², R_{B}¹² and R_{B}^{3a}, and R_{B}¹² and R_{B}^{3b}, together with the atoms to which they are attached, optionally form C₃-C₆ cycloalkyl, 3-6 membered heterocyclic.

In some embodiments, m_{B} is 1. In some embodiments, n_{B} is 1. In some embodiments, n_{B} is 2. In some embodiments, o_{B} is 0. In some embodiments, o_{B} is 1. In some embodiments, o_{B} is 2. In some embodiments, p_{B} is 0. In some embodiments, p_{B} is 1. In some embodiments, p_{B} is 2. In some embodiments, p_{B-1} is 0. In some embodiments, p_{B-1} is 1. In some embodiments, q_{B} is 0. In some embodiments, q_{B} is 1. In some embodiments, q_{B} is 2.

In some embodiments, the cGAS ligand comprise the structure of Formulae (C—I), (C—I-a), (C—I-a1), (C—I-a2), (C—I-a3), and (C—I-a4).

In some embodiments, the cGAS ligand may be a derivative of following compounds:

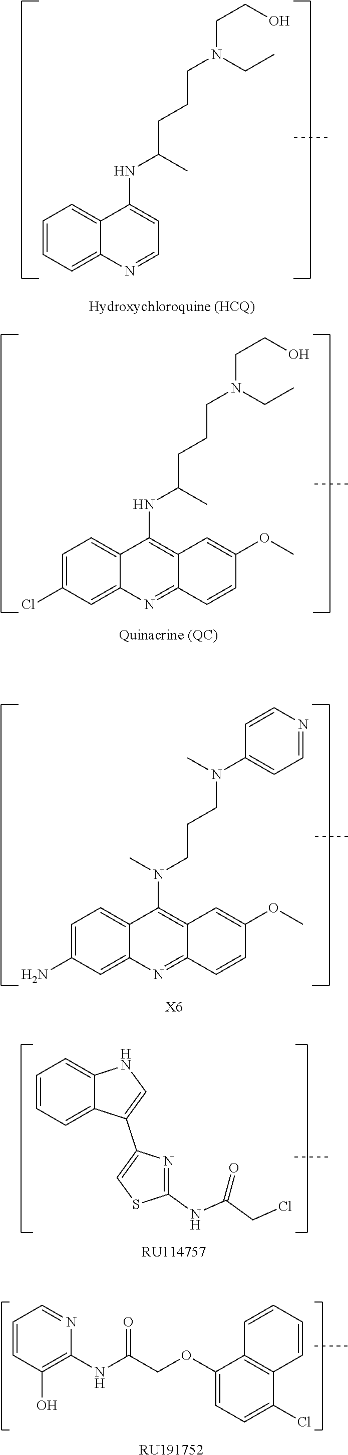

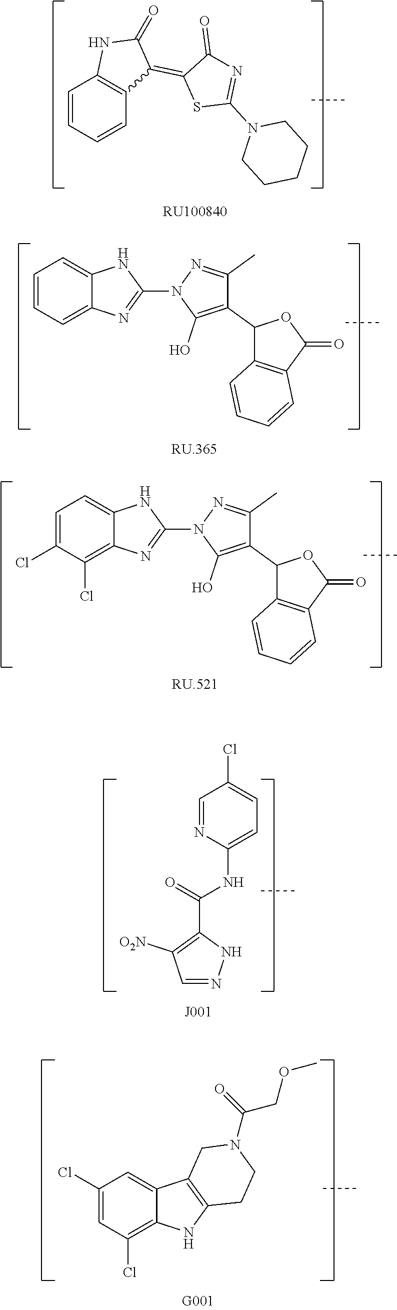

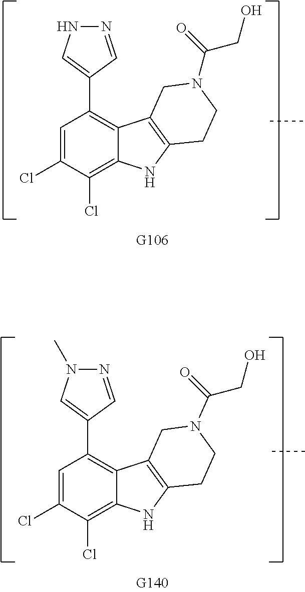

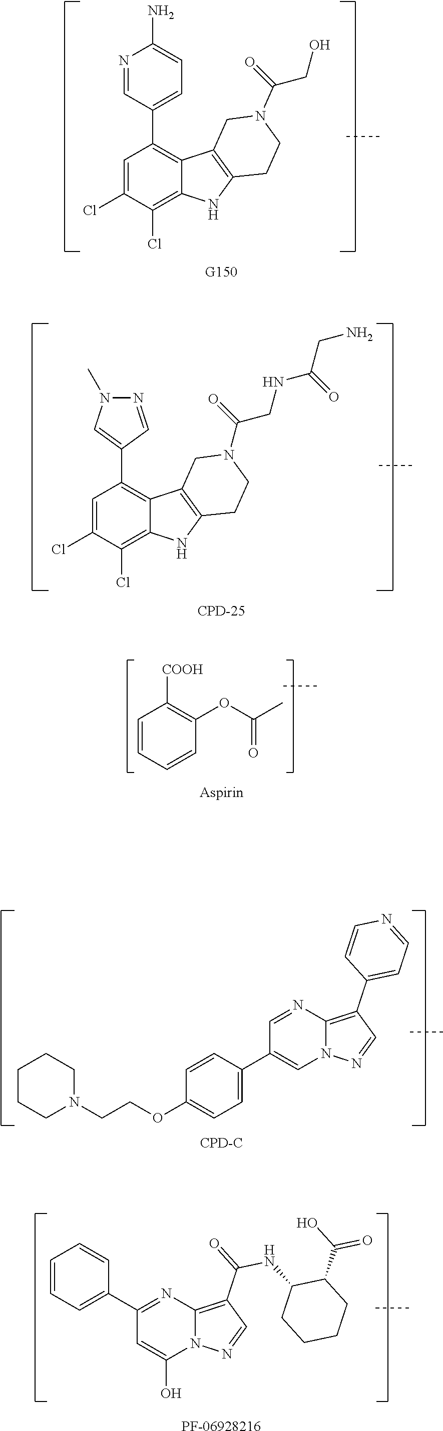

Wherein, [symbol] denotes the point of attachment to Linker.

One skilled in the art recognizes that there are sites on the ligand and recruiter that are not necessary for binding of the ligand and recruiter to the target protein and USP7, respectively, and would be appropriate for attaching a linker, provided that the linker, once attached to the ligand and recruiter does not significantly interfere with the binding of the bivalent compound to the target protein and USP7. Once a site is selected, one skilled in the art is able to chemically modify the ligand and/or recruiter to provide a derivative compound having a free valence at the desired site for attachment to a linker.

### CFTR Ligand

In some embodiments, the CFTR ligand can be a CFTR potentiator which potently binds with either wild type CFTR or mutant CFTR. In some embodiments, the CFTR ligand comprises ivacftor, lumacaftor, tezacaftor, elexacafor, or icenticaftor, or derivative thereof. In some embodiments, the CFTR ligand is a compound disclosed in one or more of U.S. Pat. Nos. 7,999,113; 8,247,436; 8,410,274; WO 2011/133953; and WO2018/037350, each of which is incorporated by reference in its entirety.

In some embodiments, the CFTR ligand comprise the structure of Formula (D-I):

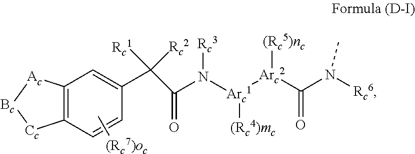

or a tautomer thereof, or a pharmaceutically acceptable salt of the compound or the tautomer,
wherein
[symbol] denotes the point of attachment to Linker in Formula (A).
A_{C} and C_{C} are independently selected from O, S, or C(R_{C}⁸)(R_{C}⁹);
B_{C} is C(R_{C}¹⁰)(R_{C}¹¹) or NR_{C}¹²;
Ar_{C}¹ and Ar_{C}² are independently selected from null, C₆-C₁₀ aryl and 5-10 membered heteroaryl;
R_{C}¹ and R_{C}² are independently selected from H, halo, OR_{C}¹³, NR_{C}¹³R_{C}¹⁴, C(O)OR_{C}¹³, C(O)NR_{C}¹³R_{C}¹⁴, S(O)₂NR_{C}¹³R_{C}¹⁴, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic, or
R_{C}¹ and R_{C}², together with the atoms to which they are attached, optionally form C₃-C₁₂ cycloalkyl;
R_{C}³ and R_{C}⁶ are independently selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic;
each R_{C}⁴, each R_{C}⁵, and each R_{C}⁷ are independently selected from H, halogen, cyano, OR_{C}¹³, NR_{C}¹³R_{C}¹⁴, C(O)OR_{C}¹³, C(O)NR_{C}¹³R_{C}¹⁴, S(O)₂NR_{C}¹³R_{C}¹⁴, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic,
two R_{C}⁴, two R_{C}⁵, or two R_{C}⁷, together with the atoms to which they are attached, optionally form partially unsaturated C₃-C₁₂ cycloalkyl, partially unsaturated 3-12 membered heterocyclic, C₆-C₁₀ aryl and 5-10 membered heteroaryl;
R_{C}⁸, R_{C}⁹, R_{C}¹⁰ and R_{C}¹¹ are independently selected from H, halogen, OR_{C}¹³, NR_{C}¹³R_{C}¹⁴, C(O)OR_{C}¹³, C(O)NR_{C}¹³R_{C}¹⁴, S(O)₂NR_{C}¹³R_{C}¹⁴, C₁-C₆ alkyl, C₁-C₆ haloalkyl, C₁-C₆ heteroalkyl, C₃-C₁₂ cycloalkyl, and 3-12 membered heterocyclic, or
R_{C}⁸ and R_{C}⁹, and R_{C}¹⁰ and R_{C}¹¹, together with the atoms to which they are attached, optionally form C₃-C₁₂ cycloalkyl and 3-12 membered heterocyclic;
R_{C}¹² is selected from H, C₁-C₆ alkyl, C₁-C₆ haloalkyl, C₁-C₆ heteroalkyl, C₃-C₁₂ cycloalkyl, and 3-12 membered heterocyclic;
R_{C}¹³ and R_{C}¹⁴ are independently selected from H, C₁-C₆ alkyl, C₁-C₆ haloalkyl, C₁-C₆ heteroalkyl, C₃-C₁₂ cycloalkyl, and 3-12 membered heterocyclic, or
R_{C}¹³ and R_{C}¹⁴ together with the atoms to which they are attached, optionally form 3-12 membered heterocyclic;
m_{C} is 0, 1, 2, or 3;
n_{C} is 0, 1, 2, or 3; and
o_{C} is 0, 1, 2, or 3.

In some embodiments, Ar_{C}¹ is 5-10 membered heteroaryl. In some embodiments, Ar_{C}¹ is selected from furyl, thiophenyl, pyrrolyl, pyrazolyl, oxazolyl, oxadiazolyl, thiazolyl, isoxazolyl, isothiazolyl, imidazolyl, triazolyl, pyridinyl, pyrimidinyl, pyrazinyl, and pyridazinyl. In some embodiments, Ar_{B} is pyridinyl. In some embodiments, Ar_{C}² is C₆-C₁₀ aryl. In some embodiments, Ar_{C}² is phenyl.

In some embodiments, the CFTR ligand comprise the structure of Formula (D-I-a):

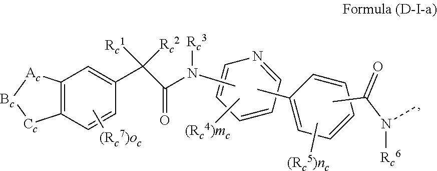

or a tautomer thereof, or a pharmaceutically acceptable salt of the compound or the tautomer.

In some embodiments, the CFTR ligand comprise the structure of Formula (D-I-a1):

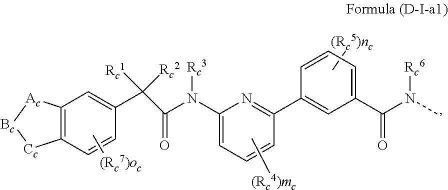

or a tautomer thereof, or a pharmaceutically acceptable salt of the compound or the tautomer.

In some embodiments, A_{C} is O. In some embodiments, B_{C} is C(R_{C}¹⁰)(R_{C}¹¹). In some embodiments, C_{C} is O.

In some embodiments, the CFTR ligand comprise the structure of Formula (D-I-a2):

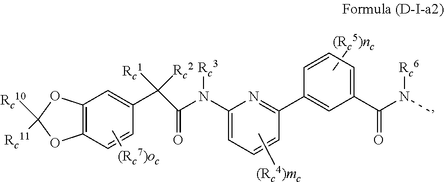

or a tautomer thereof, or a pharmaceutically acceptable salt of the compound or the tautomer.

In some embodiments, R_{C}¹ is selected from H, C₁-C₃ alkyl, C₂-C₃ alkenyl, C₂-C₃ alkynyl, C₃-C₅ cycloalkyl, and 3-5 membered heterocyclic. In some embodiments, R_{C}² is selected from H, C₁-C₃ alkyl, C₂-C₃ alkenyl, C₂-C₃ alkynyl, C₃-C₅ cycloalkyl, and 3-5 membered heterocyclic. In some embodiments, R_{C}¹ and R_{C}², together with the atoms to which they are attached, optionally form C₃-C₆ cycloalkyl. In some embodiments, R_{C}¹ and R_{C}², together with the atoms to which they are attached, optionally form cyclopropyl.

In some embodiments, R_{C}³ is selected from H, C₁-C₆ alkyl, C₁-C₆ haloalkyl, and C₁-C₆ heteroalkyl. In some embodiments, R_{C}³ is H.

In some embodiments, each R_{C}⁴ is independently selected from H, halo, cyano, OR_{C}¹³, NR_{C}¹³R_{C}¹⁴, C₁-C₆ alkyl, C₂-C₆ alkenyl, C₂-C₆ alkynyl, C₃-C₁₂ cycloalkyl, and 3-12 membered heterocyclic. In some embodiments, each R_{C}⁴ is independently selected from H and C₁-C₆ alkyl. In some embodiments, each R_{C}⁴ is independently selected from H and CH₃.

In some embodiments, each R_{C}⁵ is independently selected from H, halo, cyano, OR_{C}¹³, NR_{C}¹³R_{C}¹⁴, C₁-C₆ alkyl, C₂-C₆ alkenyl, C₂-C₆ alkynyl, C₃-C₁₂ cycloalkyl, and 3-12 membered heterocyclic. In some embodiments, each R_{C}⁵ is independently selected from H and C₁-C₆ alkyl. In some embodiments, R_{C}⁵ is H.

In some embodiments, R_{C}⁶ is selected from H, C₁-C₆ alkyl, C₁-C₆ haloalkyl, and C₁-C₆ heteroalkyl. In some embodiments, R_{C}⁶ is H. In some embodiments, R_{C}³ is CH₃.

In some embodiments, each R_{C}⁷ is independently selected from H, halogen, cyano, OR_{C}¹³, NR_{C}¹³R_{C}¹⁴, C₁-C₆ alkyl, C₂-C₆ alkenyl, C₂-C₆ alkynyl, C₃-C₁₂ cycloalkyl, and 3-12 membered heterocyclic. In some embodiments, each R_{C}⁷ is independently selected from H and C₁-C₆ alkyl. In some embodiments, R_{C}⁷ is H.

In some embodiments, R_{C}⁸ and R_{C}⁹ are independently selected from H, halogen, C₁-C₆ alkyl, C₁-C₆ haloalkyl, C₁-C₆ heteroalkyl, C₃-C₁₂ cycloalkyl, and 3-12 membered heterocyclic. In some embodiments, R_{C}⁸ and R_{C}⁹ are independently selected from H and C₁-C₆ alkyl.

In some embodiments, R_{C}¹⁰ and R_{C}¹¹ are independently selected from H, halogen, C₁-C₆ alkyl, C₁-C₆ haloalkyl, C₁-C₆ heteroalkyl, C₃-C₁₂ cycloalkyl, and 3-12 membered heterocyclic. In some embodiments, R_{C}¹⁰ and R_{C}¹¹ are independently selected from H, halogen and C₁-C₆ alkyl. In some embodiments. R_{C}¹⁰ and R_{C}¹¹ are independently selected from H, F, Cl, CH₃, and CF₃. In some embodiments, both R_{C}¹⁰ and R_{C}¹¹ are F.

In some embodiments, R_{C}¹² is selected from H, C₁-C₆ alkyl, C₁-C₆ haloalkyl, C₁-C₆ heteroalkyl, C₃-C₁₂ cycloalkyl, and 3-12 membered heterocyclic.

In some embodiments, R_{C}¹³ and R_{C}¹⁴ are independently selected from H, C₁-C₆ alkyl, C₁-C₆ haloalkyl, C₁-C₆ heteroalkyl, C₃-C₁₂ cycloalkyl, and 3-12 membered heterocyclic. In some embodiments, R_{C}¹³ and R_{C}¹⁴ are independently selected from H and C₁-C₆ alkyl. In some embodiments, R_{C}¹³ and R_{C}¹⁴ are independently selected from H and CH₃.

In some embodiments, R_{C}¹³ and R_{C}¹⁴ together with the atoms to which they are attached, optionally form 3-6 membered heterocyclic.

In some embodiments, m_{C} is 1. In some embodiments, n_{C} is 0. In some embodiments, o_{C} is 0.

In some embodiments, the CFTR ligand comprise the structure of Formulae (D-I-a3) and (D-I-a4):

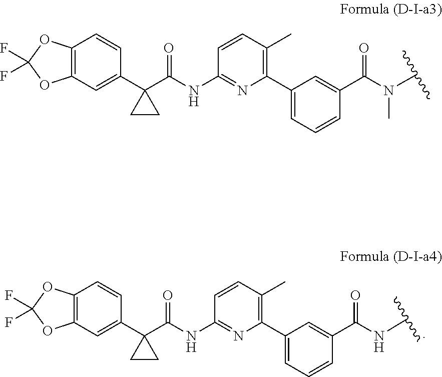 [SMILES: *N(C)C(=O)c1cccc(-c2nc(NC(=O)C3(c4ccc5c(c4)OC(F)(F)O5)CC3)ccc2C)c1 ; *NC(=O)c1cccc(-c2nc(NC(=O)C3(c4ccc5c(c4)OC(F)(F)O5)CC3)ccc2C)c1]

In some embodiments, the CFTR ligand may be a derivative of following compounds:

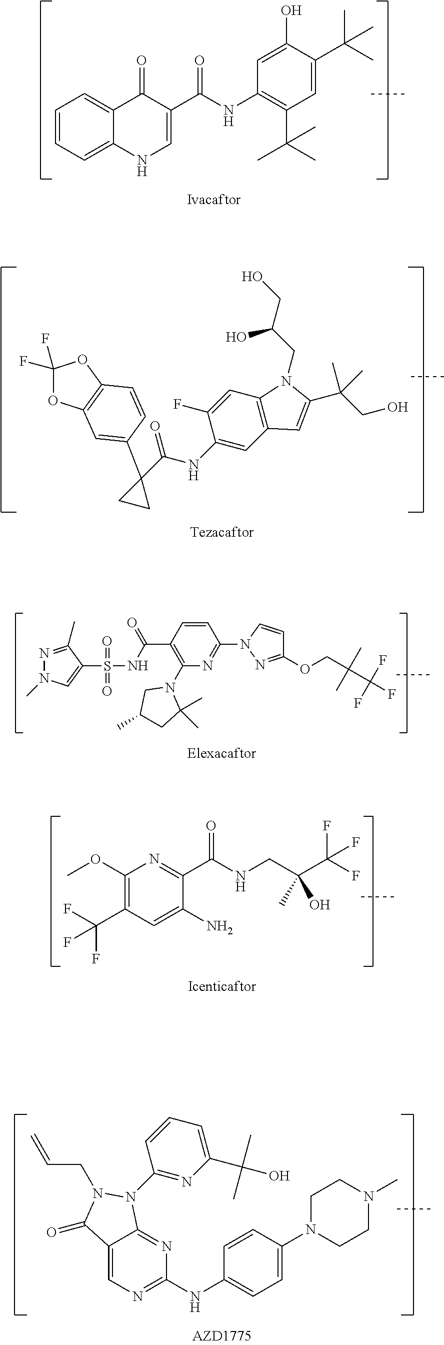

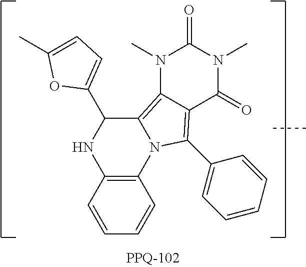

Wherein, [symbol] denotes the point of attachment to Linker.

One skilled in the art recognizes that there are sites on the ligand and recruiter that are not necessary for binding of the ligand and recruiter to the target protein and USP7, respectively, and would be appropriate for attaching a linker, provided that the linker, once attached to the ligand and recruiter does not significantly interfere with the binding of the bivalent compound to the target protein and USP7. Once a site is selected, one skilled in the art is able to chemically modify the ligand and/or recruiter to provide a derivative compound having a free valence at the desired site for attachment to a linker.

### Linkers

In all of the above-described compounds, the AMPK, cGAS or CFTR ligand is conjugated to the de-ubiquitination tag through a linker. The linker can include, for example, acyclic or cyclic saturated or unsaturated carbon, ethylene glycol, amide, amino, ether, urea, carbamate, aromatic, heteroaromatic, heterocyclic and/or carbonyl containing groups with different lengths.

In some embodiments, the linker can be a moiety of Formula (L):

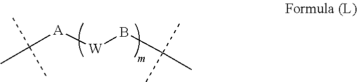

wherein
A, W and B, at each occurrence, are independently selected from null, or bivalent moiety selected from R′—R″, R′COR″, R′CO₂R″, R′C(O)N(R¹)R″, R′C(S)N(R¹)R″, R′OR″, R′OC(O)R″, R′OC(O)OR″, R′OCON(R¹)R″, R′SR″, R′SOR″, R′SO₂R″, R′SO₂N(R¹)R″, R′N(R¹)R″, R′NR¹COR″, R′NR¹C(O)OR″, R′NR¹CON(R²)R″, R′NR¹C(S)R″, R′NR¹S(O)R″, R′NR¹S(O)₂R″, and R′NR¹S(O)₂N(R²)R″, wherein
R′ and R″ are independently selected from a bond, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R^{r} is selected from optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclic, optionally substituted aryl, and optionally substituted heteroaryl;
R′ and R″, R¹ and R², R′ and R¹, R′ and R², R″ and R¹, R″ and R² together with the atom to which they are connected form a 3-20 membered cycloalkyl or 4-20 membered heterocyclic ring; and
m is 0 to 15.

In one embodiment, the linker moiety is of Formula (L-1)

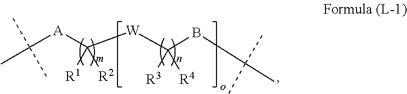

wherein
R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclic, optionally substituted aryl, and optionally substituted heteroaryl, or
R¹ and R², R³ and R⁴ together with the atom to which they are connected form a 3-20 membered cycloalkyl or 4-20 membered heterocyclic ring;
A, W and B, at each occurrence, are independently selected from null, or bivalent moiety selected from R′—R″, R′COR″, R′CO₂R″, R′C(O)N(R⁵)R″, R′C(S)N(R⁵)R″, R′OR″, R′OC(O)R″, R′OC(O)OR″, R′OCONR′R″, R′SR″, R′SOR″, R′SO₂R″, R′SO₂N(R⁵)R″, R′N(R⁵)R″, R′NR⁵COR″, R′NR⁵C(O)OR″, R′NR⁵CON(R⁶)R″, R′NR⁵C(S)R″, R′NR⁵S(O)R″, R′NR⁵S(O)₂R″, and R′NR⁵S(O)₂N(R⁶)R″, wherein
R′ and R″ are independently selected from null, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R^{r} is selected from optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclic, optionally substituted aryl, and optionally substituted heteroaryl;
R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclic ring;
m is 0 to 15;
n, at each occurrence, is 0 to 15; and
o is 0 to 15.

In another embodiment, the linker moiety is of Formula (L-2)

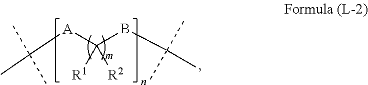

wherein
R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclic, optionally substituted aryl, and optionally substituted heteroaryl, or
R¹ and R² together with the atom to which they are connected form a 3-20 membered cycloalkyl or 4-20 membered heterocyclic ring;
A and B, at each occurrence, are independently selected from null, or bivalent moiety selected from R′—R″, R′COR″, R′CO₂R″, R′C(O)NR³R″, R′C(S)NR³R″, R′OR″, R′OC(O)R″, R′OC(O)OR″, R′OCON(R³)R″, R′SR″, R′SOR″, R′SO₂R″, R′SO₂N(R³)R″, R′N(R³)R″, R′NR³COR″, R′NR³C(O)OR″, R′NR³CON(R⁴)R′, R′NR³C(S)R″, R′NR³S(O)R″, R′NR³S(O)₂R″, and R′NR³S(O)₂N(R⁴)R″, wherein
R′ and R″ are independently selected from null, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R^{r} is selected from optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclic, optionally substituted aryl, and optionally substituted heteroaryl;
R′ and R″, R³ and R⁴, R′ and R³, R′ and R⁴, R″ and R³, R″ and R⁴ together with the atom to which they are connected form a 3-20 membered cycloalkyl or 4-20 membered heterocyclic ring;
m, at each occurrence, is 0 to 15; and
n is 0 to 15.

In another embodiment, the linker moiety is of Formula (L-3):

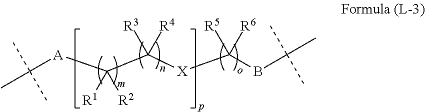

wherein
X is selected from O, NH, and NR⁷;
R¹, R², R³, R⁴, R⁵, and R⁶, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered carbocyclyl, optionally substituted 3-8 membered cycloalkoxy, optionally substituted 4-10 membered heterocyclic, optionally substituted aryl, and optionally substituted heteroaryl;
A and B are independently selected from null, or bivalent moiety selected from R′—R″, R′COR″, R′CO₂R″, R′C(O)N(R⁸)R″, R′C(S)N(R⁸)R″, R′OR″, R′OC(O)R″, R′OC(O)OR″, R′OCON(R⁸)R″, R′SR″, R′SOR″, R′SO₂R″, R′SO₂N(R⁸)R″, R′N(R⁸)R″, R′NR⁸COR″, R′NR⁸C(O)OR″, R′NR⁸CON(R⁹)R″, R′NR⁸C(S)R″, R′NR⁸S(O)R″, R′NR⁸S(O)₂R″, and R′NR⁸S(O)₂N(R⁹)R″, wherein
R′ and R″ are independently selected from null, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R^{r} is selected from optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R⁷, R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclic, optionally substituted aryl, and optionally substituted heteroaryl;
R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclic ring;
m, at each occurrence, is 0 to 15;
n, at each occurrence, is 0 to 15;
o is 0 to 15; and
p is 0 to 15.

In some embodiments, in Formula (E-3), m and n is 0 or 1, and p is 0 to 15;

In some embodiments, in Formula (E-3), X is selected from O and NH;

In some embodiments, in Formula (E-3), R¹, R², R³, R₄, R⁵, and R⁶, are independently selected from hydrogen, and optionally substituted C₁-C₆ alkyl.

In some embodiments, Formulae L, L-1, L-2, and L-3, the linker moiety comprises a ring selected from the group consisting of a 3 to 13 membered ring, a 3 to 13 membered fused ring, a 3 to 13 membered bridged ring, and a 3 to 13 membered spiro ring.

In some aspects of Formulae L, L-1, L-2, and L-3, the linker moiety comprises a ring selected from the group consisting of Formula W1, W2, W3, W4 and W5:

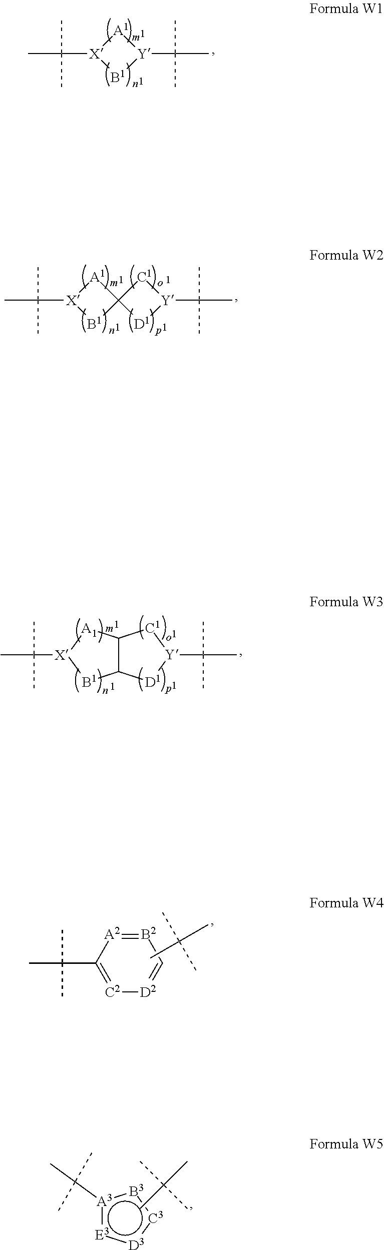

### Wherein

X and Y are independently selected from N and CR^{b};
A¹, B¹, C¹ and D¹, at each occurrence, are independently selected from null, O, CO, SO, SO₂, NR^{b}, and CR^{b}R^{c};
A², B², C² and D², at each occurrence, are independently selected from N and CR^{b};
A³, B³, C³, D³, and E³ at each occurrence, are independently selected from N, O, S, NR^{b}, and CR^{b};

R^{b} and R^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈alkylamino, optionally substituted C₁-C₈ alkylaminoC₁-C₈ alkyl, optionally substituted 3-10 membered carbocyclyl, optionally substituted 3-10 membered cycloalkoxy, optionally substituted 3-10 membered carbocyclylamino, optionally substituted 3-8 membered heterocyclic, optionally substituted aryl, and optionally substituted heteroaryl;
R^{b} and R^{b}, or R^{b} and R^{c} together with the atom to which they are connected form a 3-8 membered carbocyclyl or 3-8 membered heterocyclic ring; and
m¹, n¹, o¹ and p¹ are independently selected from 0, 1, 2, 3, 4, and 5.

In one embodiment, A, B, and W, at each occurrence, are independently selected from null, optionally substituted —(CH₂)₀₋₈—, optionally substituted —(CH₂)₀₋₈—CO—(CH₂)₀₋₈—, optionally substituted —(CH₂)₀₋₈—NH—(CH₂)₀₋₈—, optionally substituted —(CH₂)₀₋₈—NH—CO—(CH₂)₀₋₈—, optionally substituted —(CH₂)₀₋₈—CO—NH—(CH₂)₀₋₈—, optionally substituted —(CH₂)₀₋₃—NH—(CH₂)₀₋₃—CO—NH—(CH₂)₀₋₈—, optionally substituted —(CH₂)₀₋₃—NH—(CH₂)₁₋₃—NH—CO—(CH₂)₀₋₈—, optionally substituted —(CH₂)₀₋₈—CO—NH—(CH₂)₁₋₃—NH—(CH₂)₀₋₃—, optionally substituted —(CH₂)₀₋₈—R^{r}—(CH₂)₀₋₈—, optionally substituted —(CH₂)₀₋₃—CO—(CH₂)₀₋₃—R^{r}—(CH₂)₀₋₃—, —(CH₂)₀₋₈—R^{r}—(CH₂)₀₋₈—, optionally substituted —(CH₂)₀₋₃—NH—CO—(CH₂)₀₋₃—R^{r}—(CH₂)₀₋₃—, optionally substituted —(CH₂)₀₋₃—NH—(CH₂)₀₋₃—R^{r}—(CH₂)₀₋₃—.

In one embodiment, R^{r} is of Formula W1, W2, W3, W4, or W5.

In one embodiment, R^{r} is selected from

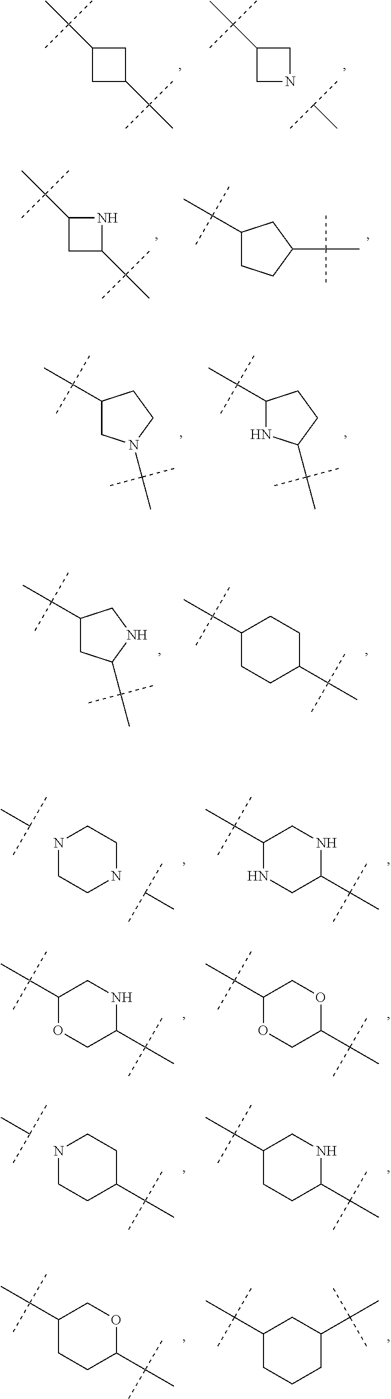

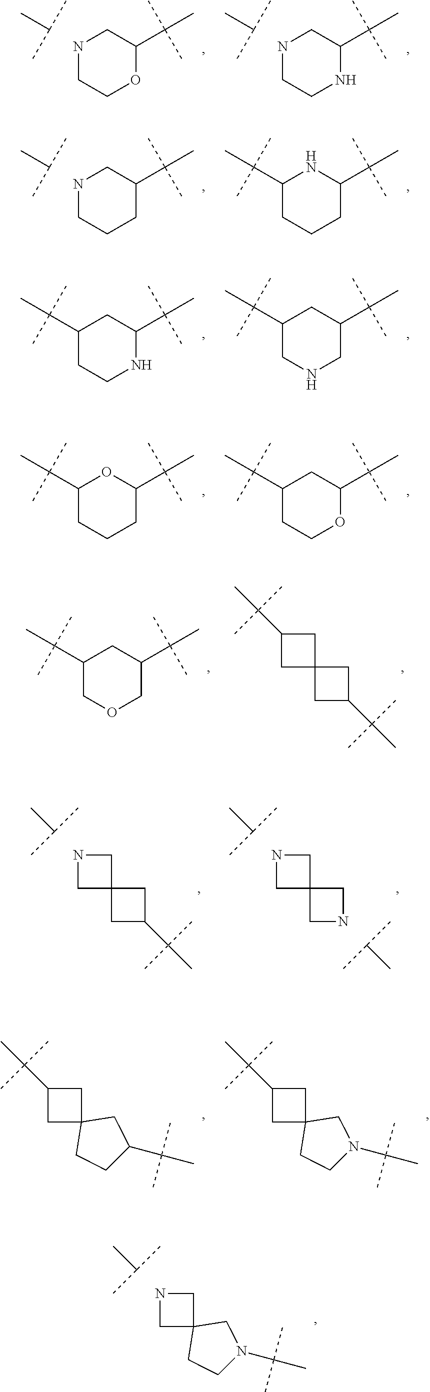

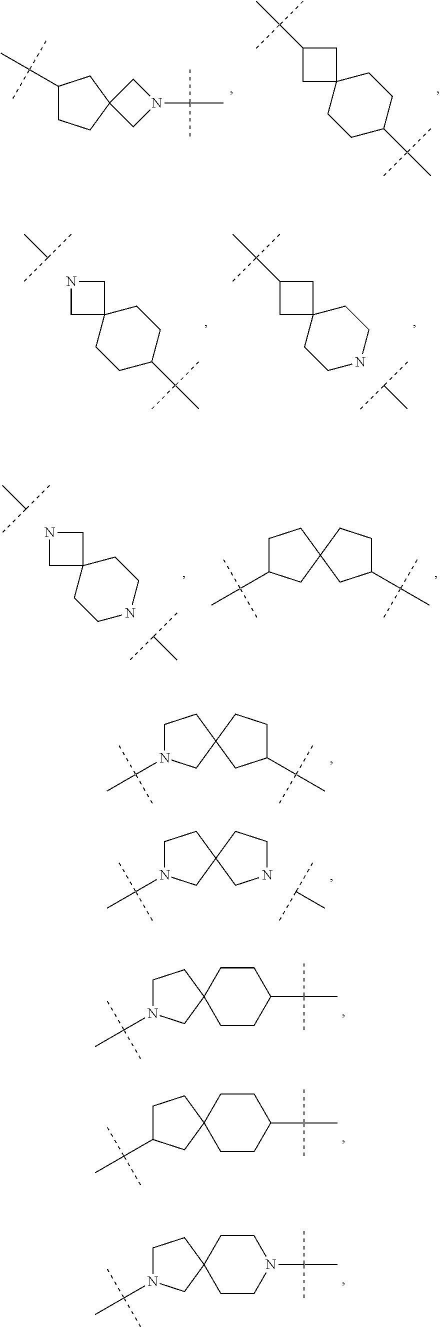

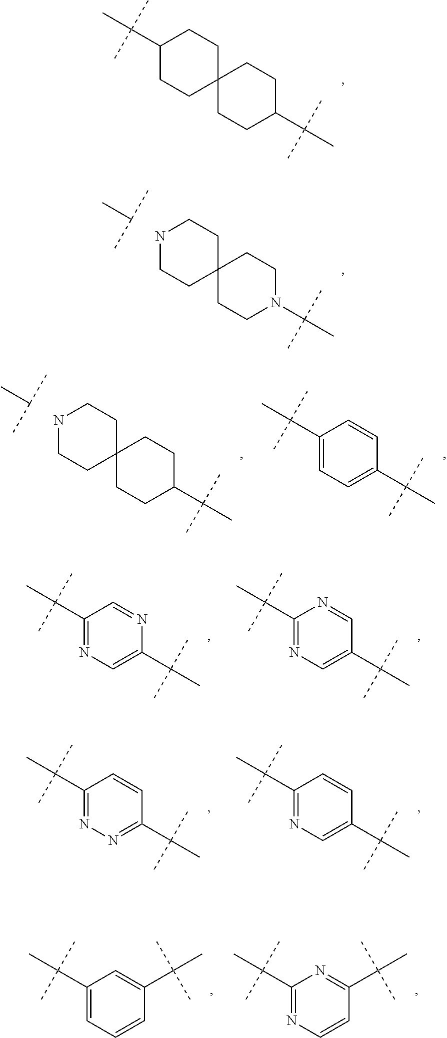

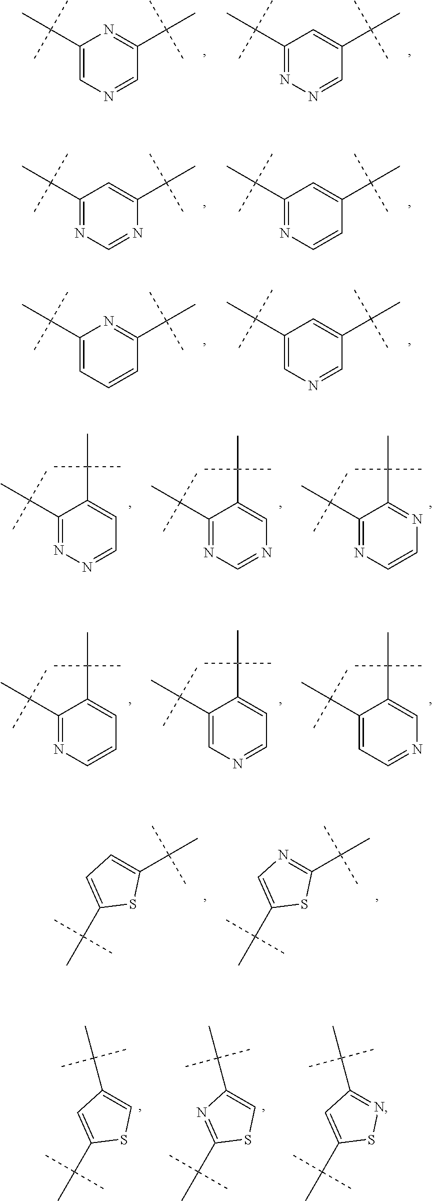

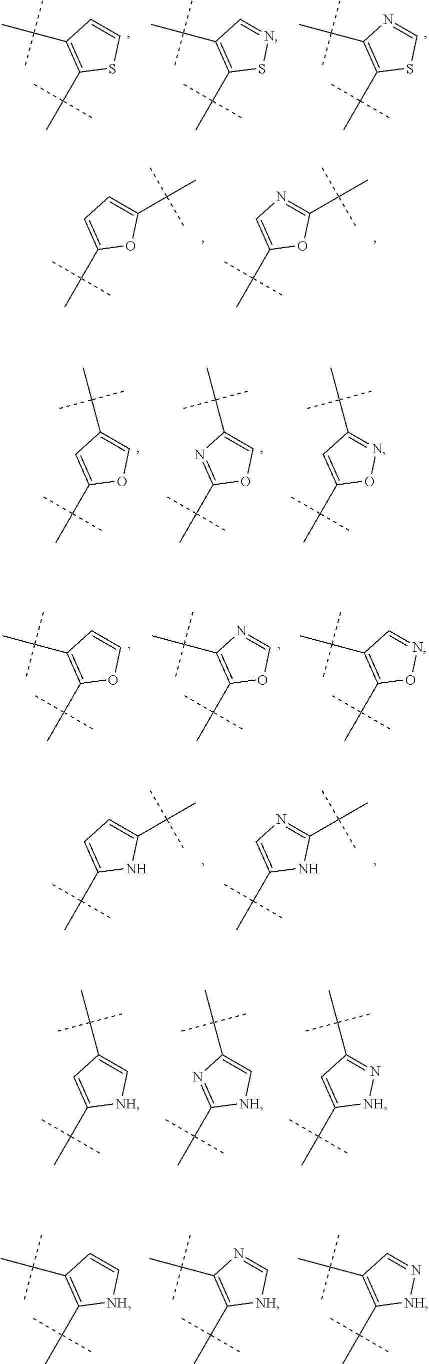

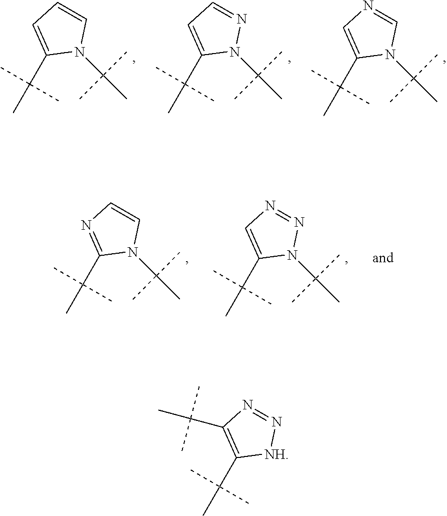

In another embodiment, the length of the linker is 0 to 40 atoms.

In another embodiment, the length of the linker is 0 to 20 atoms.

In another embodiment, the length of the linker is 0 to 10 atoms.

In another embodiment, the length of the linker is 0 to 40 atoms.

In another embodiment, the linker is selected from null, optionally substituted —CO—(CH₂)₀₋₁₀—, optionally substituted —(CH₂)₀₋₁₀—, optionally substituted —(CH₂)₁₋₂—(CO)NH—(CH₂)₀₋₁₀—, optionally substituted —(CH₂)₁₋₂—(CO)NH—(CH₂)₁₋₃—(OCH₂CH₂)₁₋₇—, optionally substituted —(CH₂)₀₋₁—CO—(CH₂)₁₋₃—(OCH₂CH₂)₁₋₇—, optionally substituted —CO—(CH₂)₀₋₃-(alkenylene)-(CH₂)₀₋₃—, optionally substituted —CO—(CH₂)₀₋₃-(alkynylene)-(CH₂)₀₋₃—, optionally substituted —CO—(CH₂)₀₋₃-(3-8 membered carbocyclyl)-(CH₂)₀₋₃—, optionally substituted —CO—(CH₂)₀₋₃-(3-8 membered heterocyclic)-(CH₂)₀₋₃—, optionally substituted —(CH₂)₀₋₃-(alkenylene)-(CH₂)₀₋₃—, optionally substituted —(CH₂)₀₋₃-(alkynylene)-(CH₂)₀₋₃—, optionally substituted —(CH₂)₀₋₃-(3-8 membered carbocyclyl)-(CH₂)₀₋₃—, optionally substituted —(CH₂)₀₋₃-(3-8 membered heterocyclic)-(CH₂)₀₋₃—, optionally substituted —(CH₂)₀₋₈—R^{r}—(CH₂)₀₋₈—, optionally substituted —(CH₂)₀₋₈—R^{r}—(CO)—(CH₂)₁₋₈—, optionally substituted —(CH₂)₀₋₈—R^{r}—(CH₂)₁₋₂—(CO)—NH—(CH₂)₂₋₉—, optionally substituted —(CH₂)₀₋₈—R^{r}—(CH₂)₁₋₂—(CO)—NH—(CH₂)₁₋₃—(OCH₂CH₂)₁₋₇—, optionally substituted —(CH₂)₀₋₈—R^{r}—(CH₂)₀₋₁—(CO)—(CH₂)₁₋₃—(OCH₂CH₂)₁₋₇—, optionally substituted —(CH₂)₀₋₈—R^{r}—(CO)—(CH₂)₀₋₃-(alkenylene)-(CH₂)₀₋₃—, optionally substituted —(CH₂)₀₋₈—R^{r}—(CO)—(CH₂)₀₋₃-(alkynylene)-(CH₂)₀₋₃—, optionally substituted —(CH₂)₀₋₈—R^{r}—(CO)—(CH₂)₀₋₃-(3-8 membered carbocyclyl)-(CH₂)₀₋₃—, optionally substituted —(CH₂)₀₋₈—R^{r}—(CO)—(CH₂)₀₋₃-(3-8 heterocyclic)-(CH₂)₀₋₃—, optionally substituted —(CH₂)₀₋₈—R^{r}—(CH₂)₀₋₃-(alkenylene)-(CH₂)₀₋₃—, optionally substituted —(CH₂)₀₋₈—R^{r}—(CH₂)₀₋₃-(alkynylene)-(CH₂)₀₋₃—, optionally substituted —(CH₂)₀₋₈—R^{r}—(CH₂)₀₋₃-(3-8 membered carbocyclyl)-(CH₂)₀₋₃—, and optionally substituted —(CH₂)₀₋₈—R^{r}—(CH₂)₀₋₃-(heterocyclic)-(CH₂)₀₋₃—.

In some embodiments, the linker can also be a moiety of:

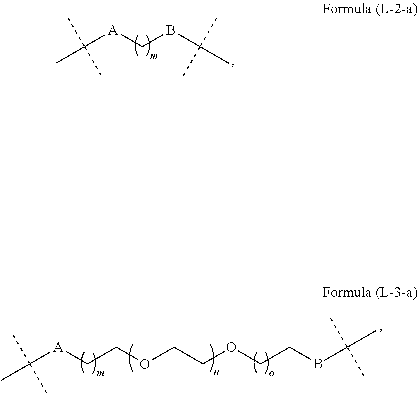

wherein
A is C═O or CH₂,
B is C═O or CH₂, and
m is 0-16,
n is 0-6, and
o is 0-6.

In some embodiments, the linker can also be a moiety of:

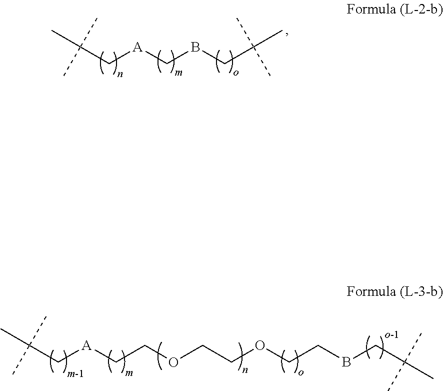

A is —C(O)N—,
B is —C(O)N—,
m is 0-16,
n is 0-6,
o is 0-6,
n−1 is 0-6, and
o−1 is 0-6.

### USP7 Recruiter

In some embodiments, the USP7 recruiter can be a USP7 binder. In some embodiments, the USP7 recruiter can be a USP7 potentiator which potently binds with USP7. In some embodiments, the USP7 recruiter comprises P5019, GNE6776, Almac4, HBX19818, P217564, XL188, XL177A, FT671, USP-797, L55, ALM34, Parthenolide, or derivative thereof. In some embodiments, the USP7 recruiter is a compound disclosed in one or more of WO2019067503; WO2013030218; CN112047933; CN113801135; WO2016109480; WO2017212010; WO2017212012; US20200095260A1; US20190142834; WO2016150800; CN111808105; WO2018073602; WO2022048498; US20200095260; WO2021175234; and WO2018183587, each of which is incorporated by reference in its entirety.

In some embodiments, the USP7 recruiter has the structure of Formula (E-I):

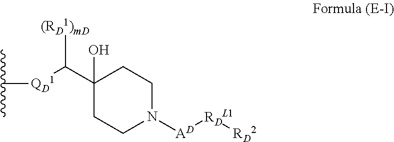

Or a pharmaceutically acceptable salt of the compound, or stereoisomer, or a tautomer thereof,
wherein
[symbol] denotes the point of attachment to Linker in Formula (A);
A_{D} is selected from C═O or C(R_{D}³)₂;
each R_{D}¹ and each R_{D}³ is independently selected from H, OH, C₁-C₆ alkyl and C₃-C₆ cycloalkyl, where each said C₁-C₆ alkyl and C₃-C₆ cycloalkyl is optionally substituted with halogen, oxo, OR_{D}⁴, NR_{D}⁴R_{D}⁵, C(O)OR_{D}⁴, C(O)NR_{D}⁴R_{D}⁵, OC(O)R_{D}⁶, NRDC(O)R_{D}⁶, S(O)₂NR_{D}⁴R_{D}⁵, NR_{D}⁴S(O)₂R_{D}⁶, C₁-C₃ alkyl, C₁-C₃ heteroalkyl, C₃-C₇ cycloalkyl, 3-7 membered heterocyclic, C₆-C₁₀ aryl, or 5-10 membered heteroaryl, or
two R_{D}¹, or two R_{D}³ together with the atom(s) to which they are attached, optionally form C₃-C₇ cycloalkyl or 3-7 membered heterocyclic;
R_{D}² is selected from hydrogen, C₁-C₆ alkyl, C₂-C₆ alkenyl, C₂-C₆ alkynyl, C₁-C₆ heteroalkyl, C₃-C₇ cycloalkyl, 3-7 membered heterocyclic, C₆-C₁₀ aryl, 5-10 membered heteroaryl, C₆-C₁₀ aryloxy, or 5-10 membered heteroaryloxy, where each said C₁-C₆ alkyl, C₂-C₆ alkenyl, C₂-C₆ alkynyl, C₁-C₆ heteroalkyl, C₃-C₇ cycloalkyl, 3-7 membered heterocyclic, C₆-C₁₀ aryl, 5-10 membered heteroaryl, C₆-C₁₀ aryloxy, 5-10 membered heteroaryloxy is optionally substituted with halogen, cyano, oxo, OR_{D}⁴, NR_{D}⁴R_{D}⁵, C(O)OR_{D}⁴, C(O)NR_{D}⁴R_{D}⁵, OC(O)R_{D}⁶, NRDC(O)R_{D}⁶, S(O)₂NR_{D}⁴R_{D}⁵, NR_{D}⁴S(O)₂R_{D}⁵, C₁-C₃ alkyl, C₁-C₃ heteroalkyl, C₃-C₇ cycloalkyl, 3-7 membered heterocyclic, C₆-C₁₀ aryl, or 5-10 membered heteroaryl;
R_{D}^{L1} is selected from a bond, C₁-C₆ alkylene, C₁-C₆ haloalkylene, C₁-C₆ heteroalkylene, C₃-C₁₂ cycloalkylene, 3-12 membered heterocyclicene, C₆-C₁₀ arylene, and 5-10 membered heteroarylene;
Q_{D}¹ is selected from nitrogen containing 5-10 membered heterocyclic and 5-10 membered heteroaryl;
each R_{D}⁴ and each R_{D}⁵ are independently selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic, or
R_{D}⁴ and R_{D}⁵, together with the atoms to which they are attached, optionally form 3-12 membered heterocyclic;
each R_{D}⁶ is selected from OR_{D}⁴, NR_{D}⁴R_{D}⁵, C₁-C₆ alkyl, C₁-C₆ haloalkyl, C₁-C₆ heteroalkyl, C₂-C₆ alkenyl, C₂-C₆ alkynyl, C₃-C₇ cycloalkyl, 3-7 membered heterocyclic, C₆-C₁₀ aryl, and 5-10 membered heteroaryl; and
m_{D} is selected from 0, 1 and 2.

In some embodiments, Q_{D}¹ is selected from formulae Q_{D}-1, Q_{D}-2, Q_{D}-3, Q_{D}-4, Q_{D}-5, Q_{D}-6 and Q_{D}-7:

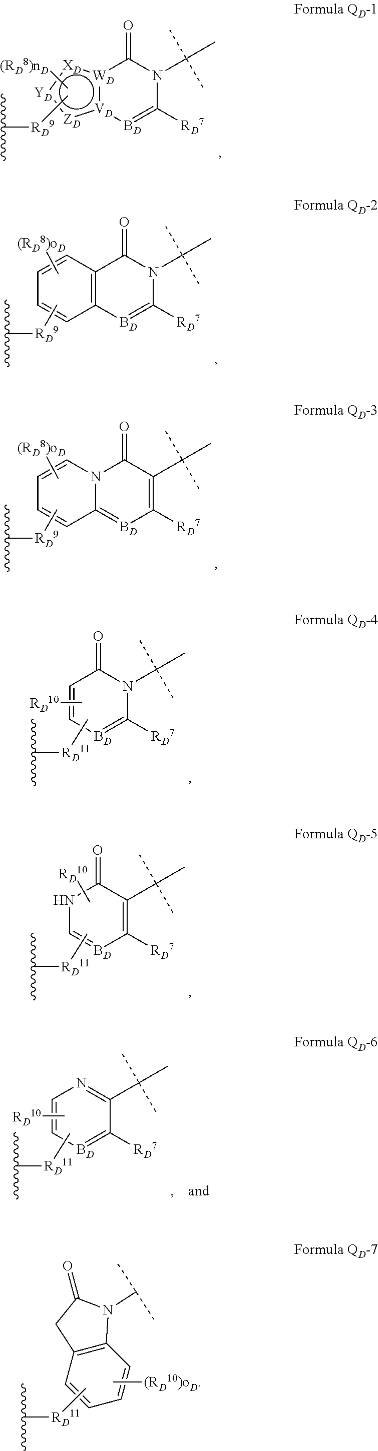

wherein
[symbol] denotes the point of attachment to Linker in Formula (A);
[symbol] denotes the point of attachment to

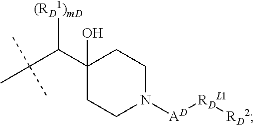

B_{D} is N or CH;
V_{D} and W_{D} are independently selected from N or C, with the proviso that at least one of V_{D} and W_{D} is C;
X_{D}, Y_{D}, and Z_{D} are independently selected from S, O, N, NR_{D}¹², and CR_{D}⁸, with the proviso that when both V_{D} and W_{D} are C, one of X_{D}, Y_{D}, and Z_{D} is S, O, or NR_{D}¹², and when one of V_{D} and W_{D} is N, none of X_{D}, Y_{D}, and Z_{D} are S, O, or NR_{D}¹²;
R_{D}⁷ is selected from hydrogen, halogen, cyano, C₁-C₃ alkyl, C₁-C₃ haloalkyl, C₁-C₃ heteroalkyl, C₃-C₅ cycloalkyl, 3-5 membered heterocyclic;
each R_{D}⁸ and each R_{D}¹⁰ are independently selected from hydrogen, halogen, cyano, OR_{D}⁴, NR_{D}⁴R_{D}⁵, OC(O)R_{D}⁶, NR_{D}⁴C(O)R_{D}⁶, C(O)R_{D}⁶, C(O)OR_{D}⁴, C(O)NR_{D}⁴R_{D}⁵, S(O)₂NR_{D}⁴R_{D}⁵, NR_{D}⁴S(O)₂R_{D}⁶, C₁-C₆ alkyl, C₂-C₆ alkenyl, C₂-C₆ alkynyl, C₁-C₆ haloalkyl, C₁-C₆ heteroalkyl, C₃-C₁₂ cycloalkyl, 3-12 membered heterocyclic, C₆-C₁₀ aryl, and 5-10 membered heteroaryl;
each R_{D}⁹ and each R_{D}¹¹ are independently selected from —R_{D}^{L2}—R_{D}^{L3}—;
R_{D}^{L2} is selected from a bond, —O—, —NR_{D}⁴—, —C(O)—, —C(O)O—, —C(O)NR_{D}⁴—, —S(O)—, —S(O)₂—, —S(O)NR_{D}⁴—, —S(O)₂NR_{D}⁴—, C₁-C₆ alkylene, C₁-C₆ haloalkylene, C₁-C₆ heteroalkylene, C₃-C₁₂ cycloalkylene, 3-12 membered heterocyclicene, C₆-C₁₀ arylene, and 5-10 membered heteroarylene;
R_{D}^{L3} is selected from a bond, —O—, —NR_{D}⁴—, C₁-C₆ alkylene, C₁-C₆ haloalkylene, C₁-C₆ heteroalkylene, C₃-C₁₂ cycloalkylene, 3-12 membered heterocyclicene, C₆-C₁₀ arylene, and 5-10 membered heteroarylene;
R_{D}¹² is selected from hydrogen, C(O)R_{D}⁶, C(O)OR_{D}⁴, C(O)NR_{D}⁴R_{D}⁵, S(O)R_{D}⁶, S(O)₂R_{D}⁶, C₁-C₆ alkyl, C₁-C₆ haloalkyl, C₁-C₆ heteroalkyl, C₃-C₁₂ cycloalkyl, 3-12 membered heterocyclic, C₆-C₁₀ aryl, and 5-10 membered heteroaryl;
R_{D}⁴, R_{D}⁵ and R_{D}⁶ are defined in Formula (E-I);
n_{D} is selected from 0, 1 and 2; and
o_{D} is selected from 0, 1, 2, 3 and 4.

In some embodiments, V_{D} is N and W_{D} is C. In some embodiments, V_{D} is C and W_{D} is N.

In some embodiments, V_{D} is C and W_{D} is C.

In some embodiments, Q_{D}-1 is selected from formulae Q_{D}-8, Q_{D}-9, and Q_{D}-10:

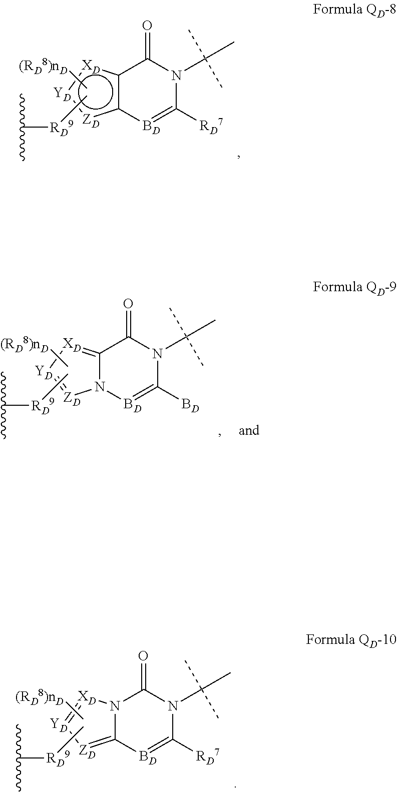

In some embodiments, Q_{D}-1 is selected from formulae Q_{D}-11, Q_{D}-12, Q_{D}-13, Q_{D}-14, Q_{D}-15, Q_{D}-16, Q_{D}-17, Q_{D}-18, Q_{D}-19, Q_{D}-20, Q_{D}-21, Q_{D}-22, Q_{D}-23, Q_{D}-24, Q_{D}-25, Q_{D}-26, Q_{D}-27, Q_{D}-28, Q_{D}-29, Q_{D}-30, Q_{D}-31, Q_{D}-32, Q_{D}-33, Q_{D}-34, Q_{D}-35, and Q_{D}-36:

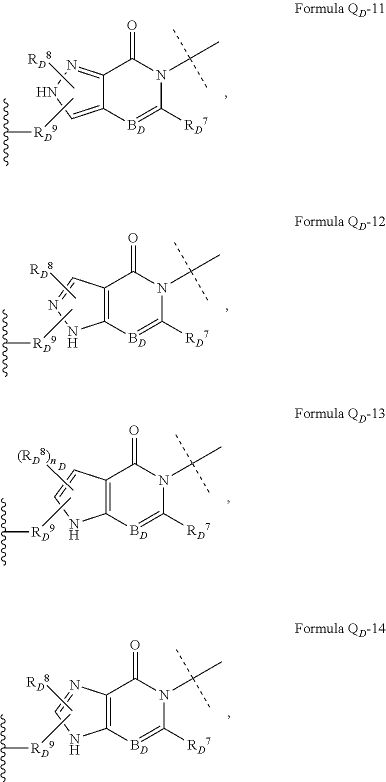

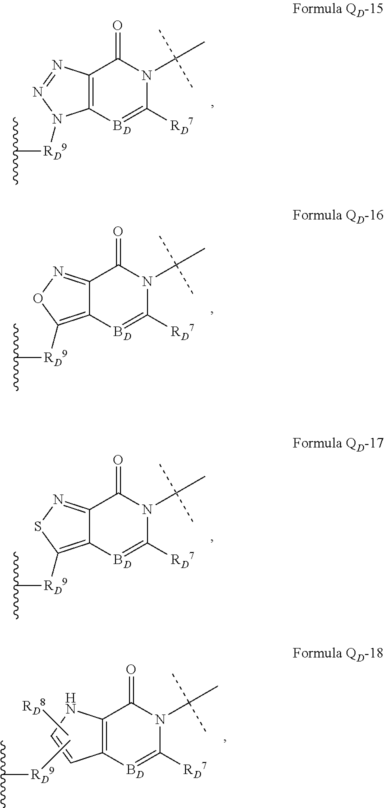

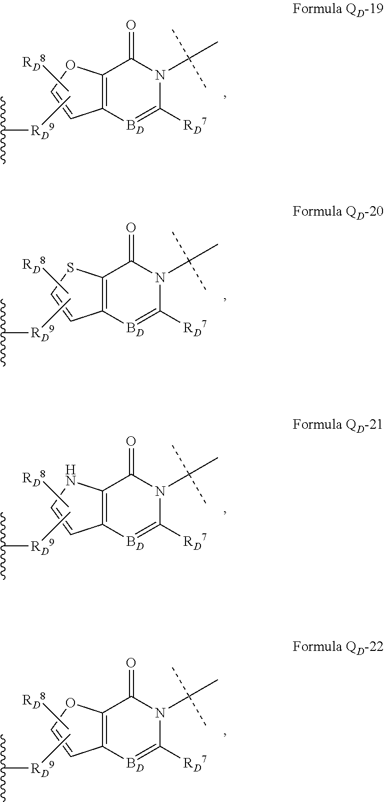

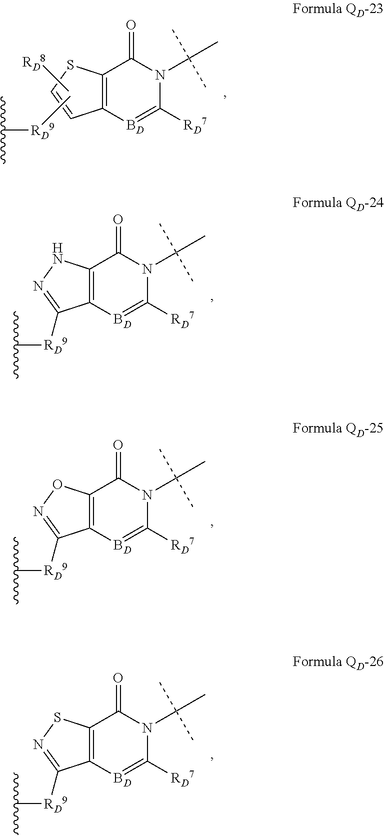

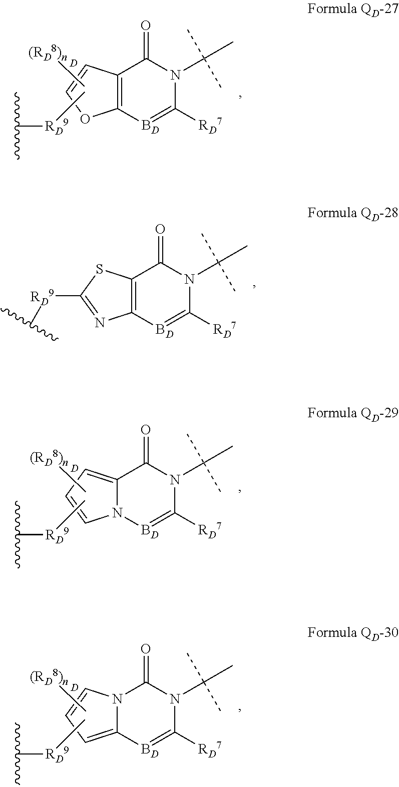

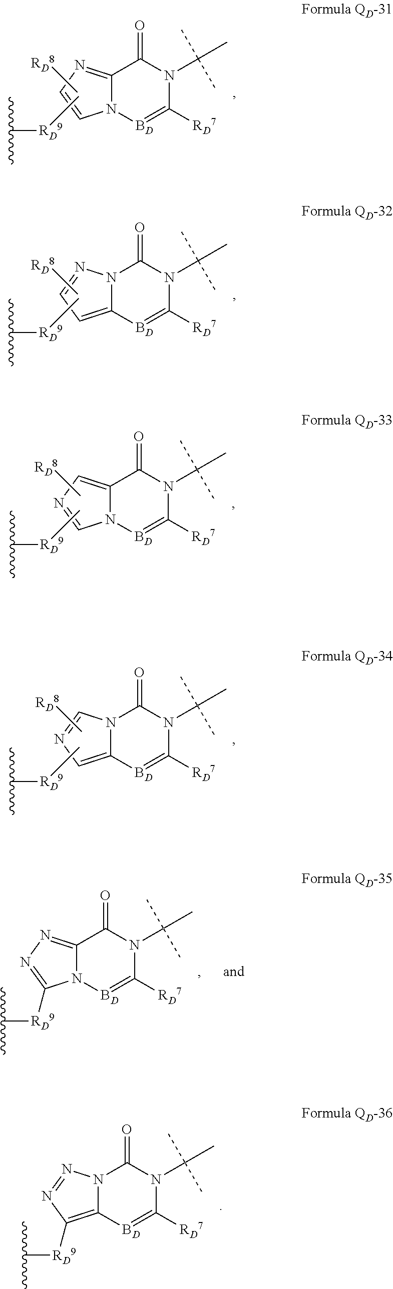

In some embodiments, Q_{D}¹ is selected from formulae Q_{D}-1, Q_{D}-2, Q_{D}-8, Q_{D}-11, Q_{D}-12, and Q_{D}-13.

In some embodiments, Q_{D}¹ is selected from formulae Q_{D}-37, Q_{D}-38, Q_{D}-39, Q_{D}-40, Q_{D}-41, Q_{D}-42:

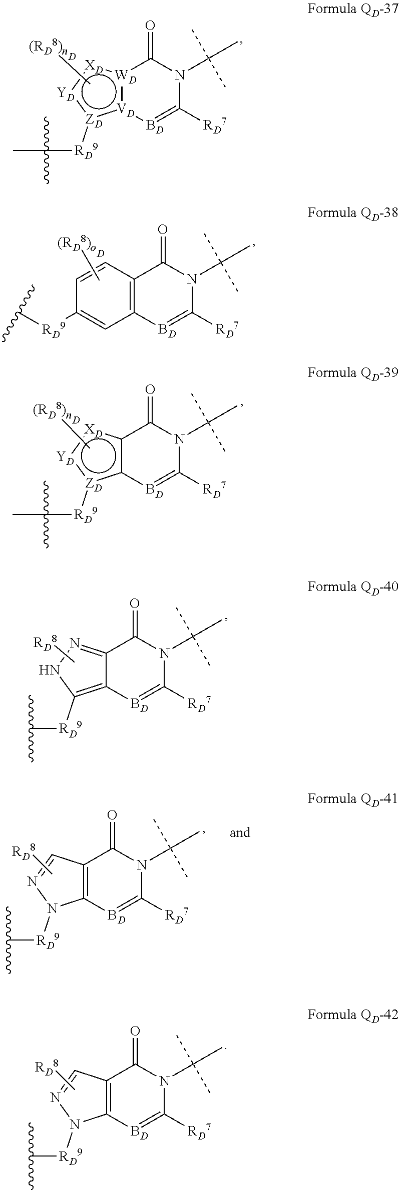

In some embodiments, B_{D} is N.

In some embodiments, R_{D}⁷ is hydrogen, C₁-C₃ alkyl, C₁-C₃ haloalkyl, C₁-C₃ heteroalkyl, and C₃-C₅ cycloalkyl. In some embodiments, R_{D}⁷ is selected from H, CH₃, CF₃, CH(CH₃)₂, and CH(CH₂)₂. In some embodiments, R_{D}⁷ is H.

In some embodiments, Q_{D}¹ is selected from formulae Q_{D}-43, Q_{D}-44, Q_{D}-45, Q_{D}-46, Q_{D}-47, Q_{D}-48:

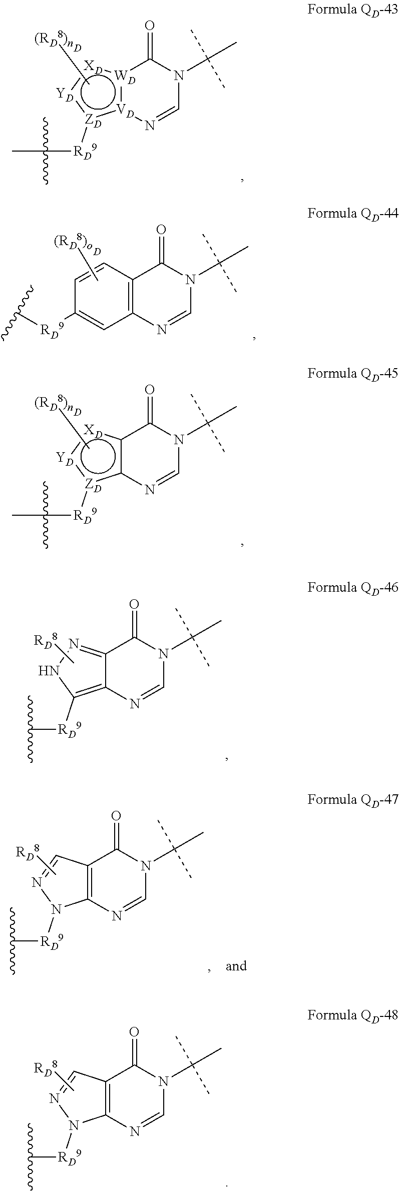

In some embodiments, each R_{D}¹ is independently selected from H, C₁-C₃ alkyl, and C₁-C₃ haloalkyl. In some embodiments, each R_{D}¹ is independently selected from H, CH₃, CF₃, CH(CH₃)₂, and CH(CH₂)₂. In some embodiments, each R_{D}¹ is independently selected from H. In some embodiments, two R_{D}¹ together with the atom(s) to which they are attached, optionally form cyclopropyl.

In some embodiments, each R_{D}³ is independently selected from H, C₁-C₃ alkyl, and C₁-C₃ haloalkyl. In some embodiments, each R_{D}³ is independently selected from H, CH₃, CF₃, CH(CH₃)₂, and CH(CH₂)₂. In some embodiments, each R_{D}³ is independently selected from H. In some embodiments, two R_{D}³ together with the atom(s) to which they are attached, optionally form cyclopropyl.

In some embodiments, A_{D} is C═O. In some embodiments, A_{D} is C(R_{D}³)₂. In some embodiments, A_{D} is CH₂.

In some embodiments, R_{D}^{L1} is selected from a bond, C₁-C₆ alkylene, C₁-C₆ haloalkylene, C₁-C₆ heteroalkylene, C₃-C₁₂ cycloalkylene, 3-12 membered heterocyclicene, C₆-C₁₀ arylene, and 5-10 membered heteroarylene. In some embodiments, R_{D}^{L1} is selected from a bond,

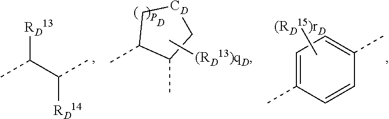

wherein
C_{D} is selected from N(R_{D}¹⁶) or C(R_{D}¹³)₂;
each R_{D}¹³ and R_{D}¹⁴ are independently selected from H, halogen, OR_{D}⁴, NR_{D}⁴R_{D}⁵, OC(O)R_{D}⁶, NR_{D}⁴C(O)R_{D}⁶, C(O)R_{D}⁶, C(O)OR_{D}⁴, C(O)NR_{D}⁴R_{D}⁵, S(O)₂NR_{D}⁴R_{D}⁵, NR_{D}⁴S(O)₂R_{D}⁶, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic, C₆-C₁₀ aryl, and 5-10 membered heteroaryl, or
R_{D}¹³ and R_{D}¹⁴, or two R_{D}¹⁴, together with the atom(s) to which they are attached, optionally form C₃-C₁₂ cycloalkyl, or 3-12 membered heterocyclic;
R_{D}¹⁵ is selected from H, halogen, cyano, nitro, OR_{D}⁴, NR_{D}⁴R_{D}⁵, OC(O)R_{D}⁶, NRDC(O)R_{D}⁶, C(O)R_{D}⁶, C(O)OR_{D}⁴, C(O)NR_{D}⁴R_{D}⁵, S(O)₂NR_{D}⁴R_{D}⁵, NR_{D}⁴S(O)₂R_{D}⁵, C₁-C₆ alkyl, C₂-C₆ alkenyl, C₂-C₆ alkynyl, C₁-C₆ haloalkyl, C₁-C₆ heteroalkyl, C₃-C₁₂ cycloalkyl, 3-12 membered heterocyclic, C₆-C₁₀ aryl, and 5-10 membered heteroaryl;
R_{D}¹⁶ is selected from hydrogen, C(O)R_{D}⁶, C(O)OR_{D}⁴, C(O)NR_{D}⁴R_{D}⁵, S(O)₂NR_{D}⁴R_{D}⁵, NR_{D}⁴S(O)₂R_{D}⁶, C₁-C₆ alkyl, C₂-C₆ alkenyl, C₂-C₆ alkynyl, C₁-C₆ haloalkyl, C₁-C₆ heteroalkyl, C₃-C₁₂ cycloalkyl, 3-12 membered heterocyclic, C₆-C₁₀ aryl, C₁-C₆ alkyl-C₆-C₁₀ aryl, 5-10 membered heteroaryl, and C₁-C₆ alkyl-5-10 membered heteroaryl;
p_{D} is selected from 0, 1, 2, and 3;
q_{D} is selected from 0, 1, 2, 3, 4, 5, and 6; and
r_{D} is selected from 0, 1, 2, 3, and 4.

In some embodiments, each R_{D}¹³ is independently selected from H, halogen, C₁-C₆ alkyl, C₁-C₆ haloalkyl, C₁-C₆ heteroalkyl, C₃-C₁₂ cycloalkyl, and 3-12 membered heterocyclic. In some embodiments, each R_{D}¹³ is independently selected from H, C₁-C₆ alkyl, C₁-C₆ haloalkyl, C₁-C₆ heteroalkyl and C₃-C₁₂ cycloalkyl. In some embodiments, R_{D}¹³ is H.

In some embodiments, R_{D}¹⁴ is selected from H, halogen, C₁-C₆ alkyl, C₁-C₆ haloalkyl, C₁-C₆ heteroalkyl, and C₃-C₁₂ cycloalkyl. In some embodiments, each R_{D}¹⁴ is independently selected from H, C₁-C₆ alkyl, C₁-C₆ haloalkyl, C₁-C₆ heteroalkyl and C₃-C₁₂ cycloalkyl. In some embodiments, R_{D}¹⁴ is selected from H, CH₃, CHF₂ and CF₃.

In some embodiments, R_{D}¹⁵ is selected from H, halogen, cyano, C₁-C₆ alkyl, C₂-C₆ alkenyl, C₂-C₆ alkynyl, C₁-C₆ haloalkyl, C₁-C₆ heteroalkyl, C₃-C₁₂ cycloalkyl, and 3-12 membered heterocyclic. In some embodiments, R_{D}¹⁵ is selected from H, F, and Cl. In some embodiments, R_{D}¹⁵ is H.

In some embodiments, R_{D}¹⁶ is selected from H, C(O)R_{D}⁶, C(O)OR_{D}⁴, C(O)NR_{D}⁴R_{D}⁵, C₁-C₆ alkyl, C₁-C₆ haloalkyl, C₁-C₆ heteroalkyl, C₃-C₁₂ cycloalkyl, 3-12 membered heterocyclic, 5-10 membered heteroaryl, and C₁-C₆ alkyl-5-10 membered heteroaryl. In some embodiments, R_{D}¹⁶ is selected from hydrogen, C(O)OR_{D}⁴, and C₁-C₆ alkyl-5-10 membered heteroaryl. In some embodiments, R_{D}¹⁶ is selected from H, C(O)OC(CH₃) 3, and

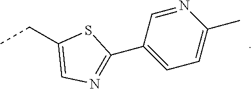

In some embodiments, R_{D}^{L1} is selected from a bond,

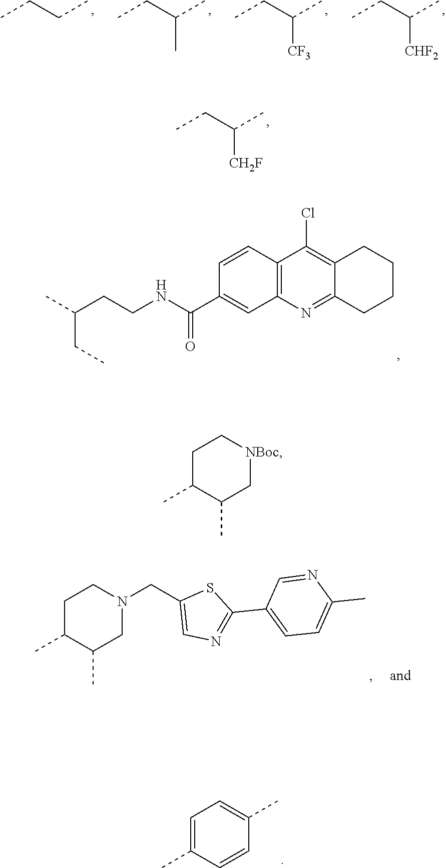

In some embodiments, R_{D}² is selected from C₆-C₁₀ aryl and 5-10 membered heteroaryl, where each said C₆-C₁₀ aryl and 5-10 membered heteroaryl is optionally substituted with halogen, cyano, oxo, OR_{D}⁴, NR_{D}⁴R_{D}⁵, C(O)OR_{D}⁴, C(O)NR_{D}⁴R_{D}⁵, OC(O)R_{D}⁶, NR_{D}⁴C(O)R_{D}⁶, S(O)₂NR_{D}⁴R_{D}⁵, NR_{D}⁴S(O)₂R_{D}⁶, C₁-C₃ alkyl, C₁-C₃ heteroalkyl, C₃-C₇ cycloalkyl, 3-7 membered heterocyclic, C₆-C₁₀ aryl, or 5-10 membered heteroaryl. In some embodiments, R_{D}² is selected from phenyl and 5-6 membered heteroaryl, where each said phenyl and 5-6 membered heteroaryl is optionally substituted with halogen, cyano, oxo, OR_{D}⁴, NR_{D}⁴R_{D}⁵, C(O)OR_{D}⁴, C(O)NR_{D}⁴R_{D}⁵, OC(O)R_{D}⁶, NR_{D}⁴C(O)R_{D}⁶, S(O)₂NR_{D}⁴R_{D}⁵, NR_{D}⁴S(O)₂R_{D}⁶, C₁-C₃ alkyl, C₁-C₃ heteroalkyl, C₃-C₇ cycloalkyl, 3-7 membered heterocyclic, C₆-C₁₀ aryl, or 5-10 membered heteroaryl. In some embodiments, R_{D}² is selected from phenyl and 5-6 membered heteroaryl, where each said phenyl and 5-6 membered heteroaryl is optionally substituted with halogen, C(O)OR_{D}⁴, C(O)NR_{D}⁴R_{D}⁵, NR_{D}⁴C(O)R_{D}⁶, S(O)₂NR_{D}⁴R_{D}⁵, NR_{D}⁴S(O)₂R_{D}⁵, C₁-C₃ alkyl, C₁-C₃ heteroalkyl, C₃-C₇ cycloalkyl, and 3-7 membered heterocyclic. In some embodiments, R_{D}² is selected from phenyl, pyridinyl and pyrazolyl, where each said phenyl, pyridinyl and pyrazolyl is optionally substituted with halogen, C(O)OR_{D}⁴, C(O)NR_{D}⁴R_{D}⁵, NRDC(O)R_{D}⁶, S(O)₂NR_{D}⁴R_{D}⁵, NR_{D}⁴S(O)₂R_{D}⁶, C₁-C₃ alkyl, C₁-C₃ heteroalkyl, C₃-C₇ cycloalkyl, and 3-7 membered heterocyclic. In some embodiments, R_{D}² is selected from phenyl, pyridinyl and pyrazolyl, where each said phenyl, pyridinyl and pyrazolyl is optionally substituted with F, Cl, Me, CF₃, CHF₂, C(O)OCH₃, C(O)NHCH₃, C(O)N(CH₃)₂, and/or NHS(O)₂CH═CH₂. In some embodiments, R_{D}² is selected from phenyl,

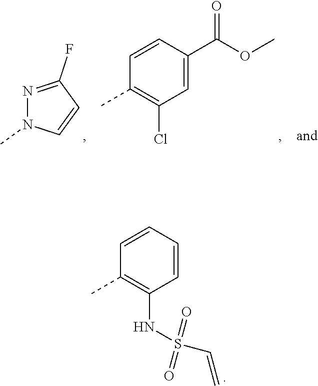

In some embodiments, each R_{D}⁴ is selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic.

In some embodiments, each R_{D}⁵ is selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic.

In some embodiments, R_{D}⁴ and R_{D}⁵, together with the atoms to which they are attached, optionally form 3-12 membered heterocyclic.

In some embodiments, each R_{D}⁶ is selected from C₁-C₆ alkyl, C₁-C₆ haloalkyl, C₁-C₆ heteroalkyl, C₂-C₆ alkenyl, C₃-C₇ cycloalkyl, 3-7 membered heterocyclic, C₆-C₁₀ aryl, and 5-10 membered heteroaryl.

In some embodiments, each R_{D}⁸ is selected from hydrogen, halogen, cyano, OR_{D}⁴, NR_{D}⁴R_{D}⁵, NR_{D}⁴C(O)R_{D}⁶, C(O)NR_{D}⁴R_{D}⁵, S(O)₂NR_{D}⁴R_{D}⁵, NR_{D}⁴S(O)₂R_{D}⁵, C₁-C₆ alkyl, C₁-C₆ haloalkyl, C₁-C₆ heteroalkyl, C₃-C₁₂ cycloalkyl, and 3-12 membered heterocyclic. In some embodiments, each R_{D}⁸ is selected from hydrogen, halogen, NR_{D}⁴C(O)R_{D}⁶, C₁-C₆ alkyl, C₁-C₆ haloalkyl, C₁-C₆ heteroalkyl, C₃-C₁₂ cycloalkyl, and 3-12 membered heterocyclic. In some embodiments, each R_{D}⁸ is selected from H, F, Cl, CH₃, and

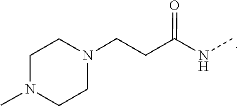

In some embodiments, R_{D}^{L2} is selected from a bond, —C(O)—, —C(O)O—, —C(O)NR_{D}⁴—, —S(O)NR_{D}⁴—, —S(O)₂NR_{D}⁴—, C₆-C₁₀ arylene, and 5-10 membered heteroarylene. In some embodiments, R_{D}^{L2} is selected from a bond, —C(O)NR_{D}⁴—, C₆-C₁₀ arylene, and 5-10 membered heteroarylene. In some embodiments, R_{D}^{L2} is selected from a bond, —NHC(O)—, and phenylene.

In some embodiments, R_{D}^{L3} is selected from a bond, —O—, —NR_{D}⁴—, C₁-C₆ alkylene, C₁-C₆ haloalkylene, C₁-C₆ heteroalkylene, C₃-C₁₂ cycloalkylene, 3-12 membered heterocyclicene. In some embodiments, R_{D}^{L3} is selected from a bond, —O—, —NH—, —CH₂—, —CH₂—CH₂—, —CH₂—NH—, and

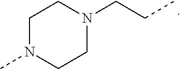

In some embodiments, each R_{D}⁹ is independently selected from —R_{D}^{L2}—R_{D}^{L3}—. In some embodiments, each R_{D}⁹ is independently selected from a bond, —NHC(O)—,

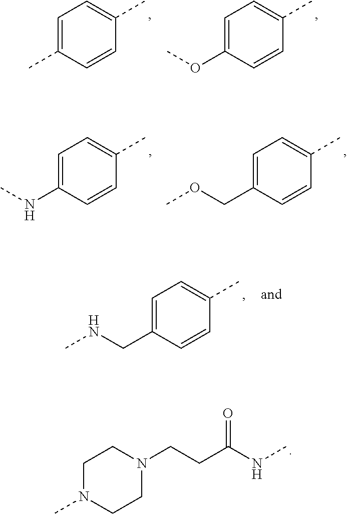

In some embodiments, R_{D}¹² is selected from hydrogen, C₁-C₆ alkyl, C₁-C₆ haloalkyl, C₁-C₆ heteroalkyl, C₃-C₁₂ cycloalkyl, and 3-12 membered heterocyclic. In some embodiments, R_{D}¹² is selected from H and CH₃.

In some embodiments, m_{D} is selected from 0 and 1. In some embodiments, m_{D} is 0. In some embodiments, n_{D} is selected from 0 and 1. In some embodiments, n_{D} is 0. In some embodiments, n_{D} is 1. In some embodiments, o_{D} is selected from 0, 1 and 2. In some embodiments, OD is selected from 0 and 1. In some embodiments, o_{D} is 0. In some embodiments, o_{D} is 1. In some embodiments, p_{D} is selected from 1 and 2. In some embodiments, p_{D} is 2. In some embodiments, q_{D} is selected from 0, 1, 2, 3, and 4. In some embodiments, q_{D} is selected from 0, 1 and 2. In some embodiments, q_{D} is 0. In some embodiments, r_{D} is selected from 0, 1, 2, 3, and 4. In some embodiments, r_{D} is selected from 0 and 1. In some embodiments, r_{D} is 0.

In some embodiments, the USP7 recruiter comprises the structure of formula (E-I-a).

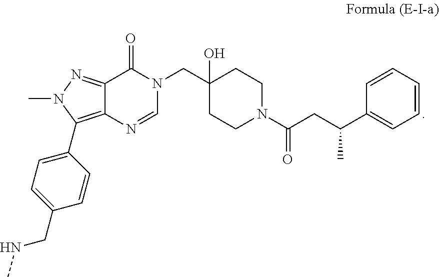

In some embodiments, the USP7 recruiter has the structure of Formula (F—I):

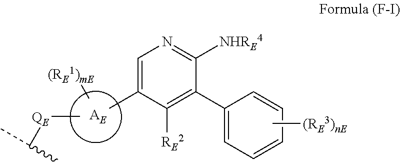

Or a tautomer, stereoisomers, or a pharmaceutically acceptable salt of the compound thereof,
wherein
[symbol] denotes the point of attachment to Linker in Formula (A);
Ring A_{E} is selected from C₃-C₁₂ cycloalkylene, 3-12 membered heterocyclicene, C₆-C₁₀ arylene, and 5-10 membered heteroarylene;
Q_{E} is selected from a bond, —O—, —NR_{E}⁵—, —C(O)—, —C(O)O—, —C(O)NR_{E}⁵—, —S(O)—, —S(O)₂—, —S(O)NR_{E}⁵—, —S(O)₂NR_{E}⁵—, C₁-C₆ alkylene, C₁-C₆ haloalkylene, C₁-C₆ heteroalkylene, C₃-C₁₂ cycloalkylene, 3-12 membered heterocyclicene, C₆-C₁₀ arylene, and 5-10 membered heteroarylene;
each R_{E}¹ and each R_{E}³ are independently selected from H, halogen, cyano, CF₃, NO₂, OR_{E}⁵, NR_{E}⁵R_{E}⁶, C(O)OR_{E}⁵, C(O)NR_{E}⁵R_{E}⁶, S(O)₂NR_{E}⁵R_{E}⁶, OC(O)R_{E}⁷, NR_{E}⁵C(O)R_{E}⁷, NR_{E}⁵S(O)₂R_{E}⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR_{E}⁸, NR_{E}⁸R_{E}⁹, C(O)OR_{E}⁸, C(O)NR_{E}⁸R_{E}⁹, OP(O)(OH)₂, OP(O)(OR_{E}⁸) (OR_{E}⁹), S(O)₂R_{E}¹⁰, S(O)₂NR_{E}⁸R_{E}⁹, OC(O)R_{E}¹⁰, NR_{E}⁸C(O)R_{E}¹⁰, NR_{E}⁸S(O)₂R_{E}¹⁰, C₁-C₆ alkyl, C₁-C₆ haloalkyl, C₁-C₆ heteroalkyl, C₃-C₁₂ cycloalkyl, 3-12 membered heterocyclic, C₆-C₁₀ aryl, and 5-10 membered heteroaryl, or
two R_{E}¹, or two R_{E}³, together with the atom(s) to which they are attached, optionally form C₃-C₆ cycloalkyl or 4-7 membered heterocyclic, where each said C₃-C₆ cycloalkyl or 4-7 membered heterocyclic is optionally substituted with halogen or C₁-C₆ alkyl;
R_{E}² is selected from H, cyano, C₁-C₃ alkyl, C₁-C₃ heteroalkyl, or C₃-C₆ cycloalkyl, where each said C₁-C₃ alkyl, C₁-C₃ heteroalkyl, and C₃-C₆ cycloalkyl, is optionally substituted with halogen, especially F;
R_{E}⁴ is selected from H, C₁-C₃ alkyl, C₁-C₃ heteroalkyl, or C₃-C₆ cycloalkyl, where each said C₁-C₃ alkyl, C₁-C₃ heteroalkyl, and C₃-C₆ cycloalkyl, is optionally substituted with halogen;
each R_{E}⁵, each R_{E}⁶, each R_{E}⁸, and each R_{E}⁹, are independently selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic, or
R_{E}⁵ and R_{E}⁶, or R_{E}⁸ and R_{E}⁹ together with the atom(s) to which they are attached, optionally form 3-12 membered heterocyclic;
each R_{E}⁷, and each R_{E}¹⁰ are independently selected from OR_{E}⁵, NR_{E}⁵R_{E}⁶, C₁-C₆ alkyl, C₁-C₆ haloalkyl, C₁-C₆ heteroalkyl, C₂-C₆ alkenyl, C₂-C₆ alkynyl, C₃-C₇ cycloalkyl, 3-7 membered heterocyclic, C₆-C₁₀ aryl, and 5-10 membered heteroaryl;
m_{E} is selected from 0, 1, 2, 3, 4, and 5; and
n_{E} is selected from 0, 1, 2, 3, 4, 5, and 6.

In some embodiments, Ring A_{E} is selected from C₆-C₁₀ arylene and 5-10 membered heteroarylene. In some embodiments, Ring A_{E} is selected from phenylene, pyridinylene, pyrimidinylene, furylene, thiophenylene, pyrrolylene, pyrazolylene, 1H-benzo[d]imidazolylene, indolylene, benzo[b]thiophenylene, or indazolyene. In some embodiments, Ring A_{E} is selected from phenylene, pyridinylene, and indazolyene. In some embodiments, Ring A_{E} is phenylene. In some embodiments, Ring A_{E} is pyridinylene. In some embodiments, Ring A_{E} is indazolyene.

In some embodiments, Q_{E} is selected from a bond, —O—, —NR_{E}⁵—, —C(O)NR_{E}⁵—, —S(O)NR_{E}⁵—, —S(O)₂NR_{E}⁵—, C₁-C₆ alkylene, C₁-C₆ haloalkylene, C₁-C₆ heteroalkylene, C₃-C₁₂ cycloalkylene, and 3-12 membered heterocyclicene. In some embodiments, Q_{E} is selected from a bond, —C(O)NR_{E}⁵—. In some embodiments, Q_{E} is selected from a bond, —C(O)NH—. In some embodiments, Q_{E} is a bond. In some embodiments, Q_{E} is —C(O)NH—.

In some embodiments, the USP7 recruiter comprises the structure of Formulae (F—I-a) and (F—I-b):

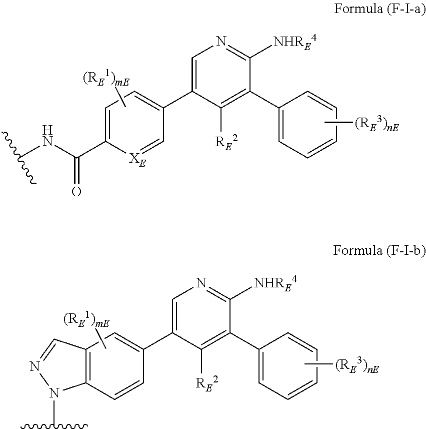

Or a tautomer, stereoisomers, or a pharmaceutically acceptable salt of the compound thereof,
wherein
[symbol] denotes the point of attachment to Linker in Formula (A), wherein:
X_{E} is selected from C(R_{E}¹) and N; and
R_{E}¹, R_{E}², R_{E}³, R_{E}⁴, m_{E} and n_{E} are defined in Formula (F—I).

In some embodiments, each R_{E}¹ is independently selected from H, halogen, cyano, CF₃, C₁-C₆ alkyl, C₁-C₆ heteroalkyl, C₃-C₁₂ cycloalkyl, and 3-12 membered heterocyclic, where each said C₁-C₆ alkyl, C₁-C₆ heteroalkyl, C₃-C₁₂ cycloalkyl, and 3-12 membered heterocyclic is optionally substituted with halogen, cyano, oxo, OR_{E}⁸, NR_{E}⁸R_{E}⁹, C(O)OR_{E}⁸, C(O)NR_{E}⁸R_{E}⁹, OP(O)(OH)₂, OP(O)(OR_{E}⁸) (OR_{E}⁹), S(O)₂R_{E}¹⁰, S(O)₂NR_{E}⁸R_{E}⁹, OC(O)R_{E}¹⁰, NR_{E}⁸C(O)R_{E}¹⁰, NR_{E}⁸S(O)₂R_{E}¹⁰, C₁-C₆ alkyl, C₁-C₆ haloalkyl, C₁-C₆ heteroalkyl, C₃-C₁₂ cycloalkyl, 3-12 membered heterocyclic, C₆-C₁₀ aryl, or 5-10 membered heteroaryl. In some embodiments, each R_{E}¹ is independently selected from H, halogen, cyano, CF₃, C₁-C₆ alkyl, C₁-C₆ haloalkyl, C₁-C₆ heteroalkyl, C₃-C₁₂ cycloalkyl, and 3-12 membered heterocyclic. In some embodiments, each R_{E}¹ is independently selected from H, halogen, cyano, CF₃, C₁-C₆ alkyl, C₁-C₆ haloalkyl. In some embodiments, each R_{E}¹ is independently selected from H, F, Cl, Br, CH₃, CF₃, and cyano. In some embodiments, R_{E}¹ is H. In some embodiments, R_{E}¹ is F, Cl, Br. In some embodiments R_{E}¹ is CH₃. In some embodiments, R_{E}¹ is CF₃. In some embodiments R_{E}¹ is cyano.

In some embodiments, R_{E}² is selected from H, cyano, C₁-C₃ alkyl, C₁-C₃ haloalkyl, C₁-C₃ heteroalkyl, or C₃-C₆ cycloalkyl. In some embodiments, R_{E}² is selected from H, CF₃, CH₃, CH₂CH₃, CH(CH₂)₂, CH₂CH₂CH₃, CH(CH₃)₂, and CH₃₀. In some embodiments, R_{E}² is H. In some embodiments, R_{E}² is CH₃. In some embodiments, R_{E}² is CH₂CH₃. In some embodiments, R_{E}² is CH(CH₂)₂. In some embodiments, R_{E}² is CF₃. In some embodiments, R_{E}² is CH₂CH₂CH₃. In some embodiments, R_{E}² is CH(CH₃)₂. In some embodiments, R_{E}² is CH₃O.

In some embodiments, each R_{E}³ are independently selected from H, halogen, cyano, CF₃, ORES, NR_{E}⁵R_{E}⁶, C₁-C₆ alkyl, C₁-C₆ haloalkyl, C₁-C₆ heteroalkyl, C₃-C₁₂ cycloalkyl, and 3-12 membered heterocyclic. In some embodiments, each R_{E}³ are independently selected from H, OH, NH₂, F, Cl, CN, CF₃, CH₃, CH(CH₂)₂, and CH(CH₃)₂. In some embodiments, R_{E}³ is OH. In some embodiments, R_{E}³ is NH₂.

In some embodiments, R_{E}⁴ is selected from H, C₁-C₃ alkyl, C₁-C₃ haloalkyl, C₁-C₃ heteroalkyl, or C₃-C₆ cycloalkyl. In some embodiments, R_{E}⁴ is selected from H, CH₃, and CH₂CH₃. In some embodiments, R_{E}⁴ is H. In some embodiments, R_{E}⁴ is CH₃. In some embodiments, R_{E}⁴ is CH₂CH₃.

In some embodiments, the USP7 recruiter comprises the structure of Formulae (F—I-a1), (F—I-a2) and (F—I-b1):

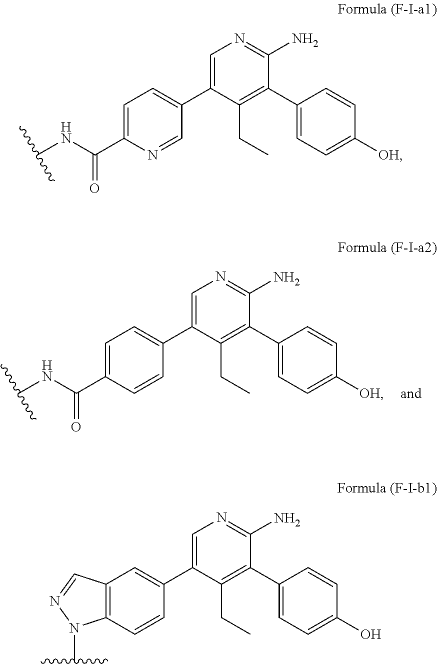 [SMILES: *NC(=O)c1ccc(-c2cnc(N)c(-c3ccc(O)cc3)c2CC)cn1 ; *NC(=O)c1ccc(-c2cnc(N)c(-c3ccc(O)cc3)c2CC)cc1 ; "and" ; *n1ncc2cc(-c3cnc(N)c(-c4ccc(O)cc4)c3CC)ccc21]

In some embodiments, the USP7 recruiter may be a derivative of following compounds: ALM34 (Gavory et al., 2018), FT671 (Turnbull et al., 2017), L55 (Li et al., 2020), GNE-6776 (Kategaya et al., 2017), GNE-6640 (Di Lello et al., 2017), HBX19818 (Reverdy et al., 2012), XL177A (Schauer et al., 2020), FT827 (Turnbull et al., 2017), NSC632839 (Nicholson et al., 2008), compound 14 (Weinstock et al., 2012), Example 14 (Pepe and Mesecar, 2018), compound 5 (O'Dowd et al., 2018), HBX41108 (Colland et al., 2009), Example 40 (Kotschy et al., 2017), Example 25 (Kotschy et al., 2017), USP7-797 (Leger et al., 2020), compound 55 (Sun et al., 2021), XL188 (Lamberto et al., 2017), Example 187 (Hou et al., 2022).

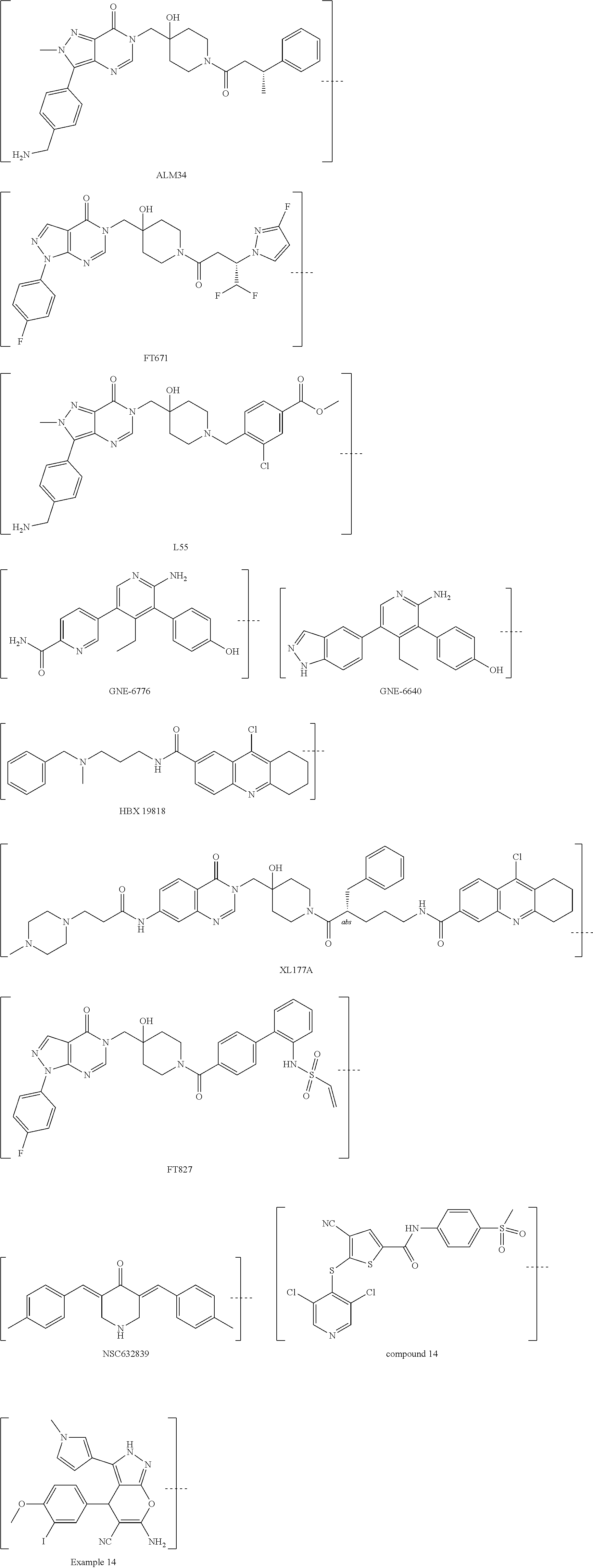

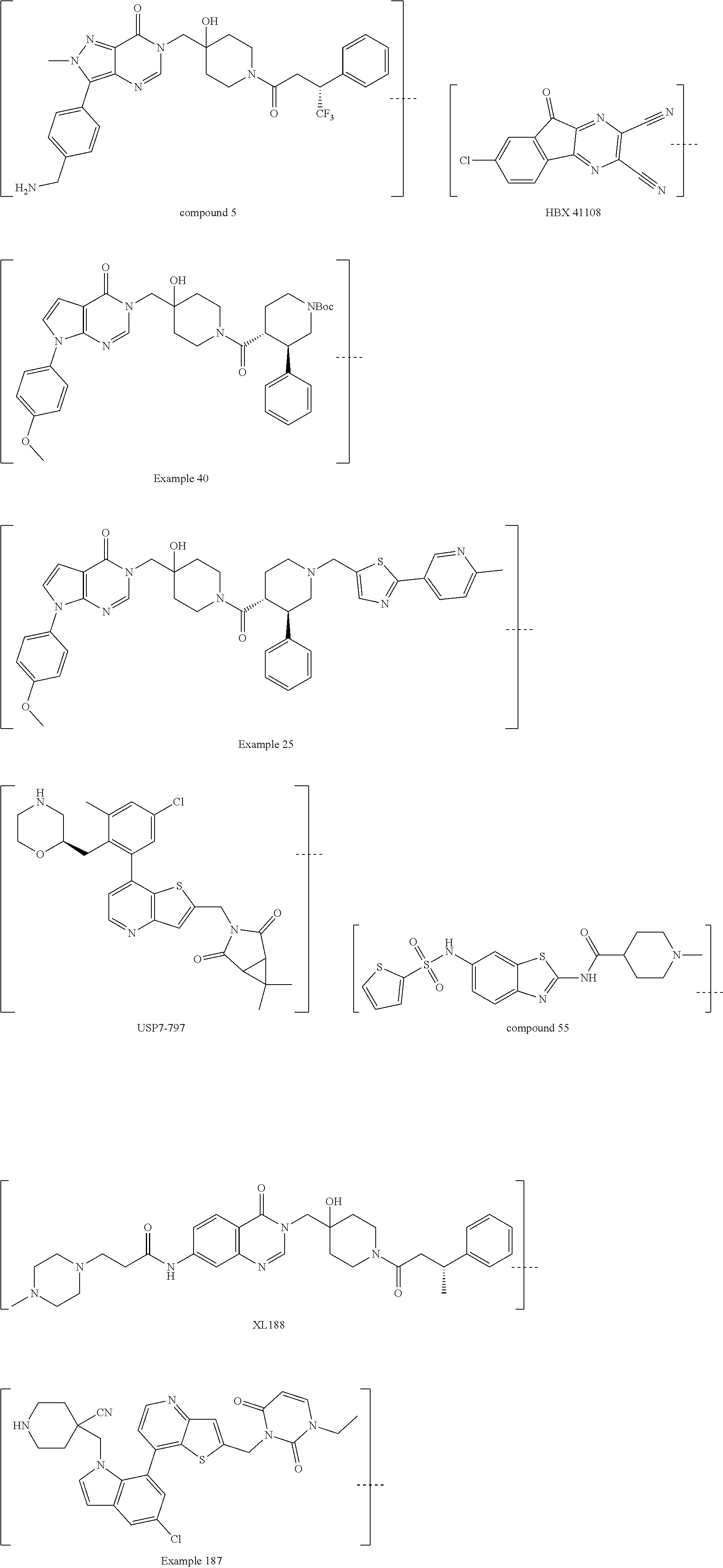

Wherein, [symbol] denotes the point of attachment to Linker.

One skilled in the art recognizes that there are sites on the ligand and recruiter that are not necessary for binding of the ligand and recruiter to the target protein and USP7, respectively, and would be appropriate for attaching a linker, provided that the linker, once attached to the ligand and recruiter does not significantly interfere with the binding of the bivalent compound to the target protein and USP7. Once a site is selected, one skilled in the art is able to chemically modify the ligand and/or recruiter to provide a derivative compound having a free valence at the desired site for attachment to a linker.

In some aspect, the USP7 recruiting AMPK DUBTACs are compounds selected from the following compounds, as identified in table 1 below: XH181-1, XH181-2, XH181-3, XH181-4, XH181-5, XH181-6, XH181-7, XH181-8, XH181-9, XH181-15, XH188-16, XH188-17, XH188-18, XH188-19, KL186-1, KL186-2, KL186-3, KL186-4, KL186-5, KL186-6, KL186-7, KL186-8, KL186-9, KL186-10, KL186-11, KL186-12, KL186-13, KL186-14, KL186-48, KL186-49, KL186-50, KL186-51, KL186-52, KL186-53, KL186-54, KL186-55, KL186-56, KL186-57, KL186-58, KL186-59, KL186-60 and analogs thereof.

In some aspects, the document provides a method of treating a AMPK-mediated diseases. The method includes administering to a subject in need thereof with a AMPK-mediated diseases one or more compounds to stabilize and activate AMPK. The AMPK-mediated disease may be a cancer or other diseases, including metabolic syndrome, type-2 diabetes, non-alcoholic fatty liver disease and cardiovascular disease. The AMPK-mediated disease with lower expression level of AMPK or unactivated AMPK relative to a wild-type tissue of the same species and tissue type. The AMPK-mediated diseases can have reduced AMPK enzymatic activity relative to a wildtype tissue of the same species and tissue type. Non-limiting examples of AMPK-mediated cancer include mesothelioma, hepatocellular cancer, central nervous system neoplasm, lung cancer, bone cancer, pancreatic cancer, skin cancer, head and neck cancer, melanoma, ovarian cancer, colon cancer, rectal cancer, anal cancer, stomach cancer, gastrointestinal cancer, breast cancer (e.g., estrogen receptor positive (ER+) breast cancer), uterine cancer, fallopian tube cancer, endometrial cancer, cervical cancer, vaginal cancer, vulvar cancer, esophageal cancer, gastrointestinal cancer, endocrine cancer, thyroid cancer, parathyroid cancer, adrenal cancer, soft tissue sarcoma, urethral cancer, penile cancer, prostate cancer, testicular cancer, leukemia, lymphoma, bladder cancer, renal cell cancer, brain stem glioma, pituitary cancer, adrenocortical cancer, gallbladder cancer, multiple myeloma, cholangiocarcinoma, fibrosarcoma, neuroblastoma, and/or retinoblastoma. The AMPK-mediated cancer can be a relapsed cancer. The AMPK-mediated cancer can have been refractory to one or more previous treatments.

In some aspects, the USP7 recruiting cGAS DUBTAC are compounds selected from the following compounds, as identified in table 2 below: XH168-142, XH168-143, XH168-144, XH168-145, XH168-146, XH168-147, XH168-148, XH168-149, XH168-150, XH168-151, XH168-152, XH168-153, XH168-154, XH168-155, XH181-72, XH181-73, XH181-90, XH181-91, XH181-92, XH181-93, XH181-94, XH181-95, XH181-96, XH181-97, XH181-99, XH181-100, XH181-101, XH181-102, XH181-103, XH188-48, XH188-49, XH188-50, XH188-51, Xh188-52, XH188-53, XH188-54, XH188-55, XH188-56, XH188-57, XH188-58, XH188-59, XH188-60, XH188-90, XH188-91, XH188-92, XH188-93, XH188-94, XH188-95, XH188-96, XH188-97, XH188-98, XH188-99, XH188-100, XH188-101, XH188-102, KL186-107, KL186-115, KL186-116, KL186-117, KL186-118, KL186-119, KL186-120, KL186-121, KL186-122, KL186-123, KL186-124, KL186-125, KL186-126, KL186-127, KL186-128, KL186-129, KL186-130, KL186-131, KL186-132, KL186-133, KL186-134, KL186-135, KL186-136, KL186-137, KL186-138, KL186-139, KL186-140, KL186-141, KL186-142, KL186-143, KL186-144, KL186-145, KL186-146, KL186-147, KL186-148, KL186-149, v50, KL186-151, KL186-152, KL186-153, KL186-154, KL186-155, KL186-156, KL186-157, KL186-158, KL186-159, KL186-160, KL186-161, KL186-162, KL186-163, KL186-164, KL186-165, KL186-166, KL186-167, KL186-168, KL186-169, KL186-170, KL186-171 and analogs thereof.

cGAS-STING functions as an innate immune signaling pathway that recognizes perturbation of cellular homeostasis and triggers potent antitumor immune responses. Therefore, in some aspects, the document provides a method of treating various disease including cancer by activating cGAS-STING signaling pathway through the stabilization and activation of cGAS. Non-limiting examples of cGAS-STING targeting cancer include mesothelioma, hepatocellular cancer, central nervous system neoplasm, lung cancer, bone cancer, pancreatic cancer, skin cancer, head and neck cancer, melanoma, ovarian cancer, colon cancer, rectal cancer, anal cancer, stomach cancer, gastrointestinal cancer, breast cancer (e.g., estrogen receptor positive (ER+) breast cancer), uterine cancer, fallopian tube cancer, endometrial cancer, cervical cancer, vaginal cancer, vulvar cancer, esophageal cancer, gastrointestinal cancer, endocrine cancer, thyroid cancer, parathyroid cancer, adrenal cancer, soft tissue sarcoma, urethral cancer, penile cancer, prostate cancer, testicular cancer, leukemia, lymphoma, bladder cancer, renal cell cancer, brain stem glioma, cancer, gallbladder cancer, multiple myeloma, pituitary cancer, adrenocortical cholangiocarcinoma, fibrosarcoma, neuroblastoma, and/or retinoblastoma. The cGAS-STING targeting cancer can be a relapsed cancer. The cGAS-STING targeting cancer can have been refractory to one or more previous treatments.

In some aspects, the USP7 recruiting CFTR DUBTAC are compounds selected from the following compounds, as identified in table 3 below: XH168-51, XH168-32, XH168-33, XH168-59, XH168-66, XH168-67, XH168-68, XH168-69, XH168-70, XH168-58, XH168-47, XH168-48, XH168-49, XH168-50, KL180-124, KL180-125, KL180-126, KL180-127, KL180-128, KL180-129, KL180-130, KL180-131, KL180-132, KL180-133, KL180-134, KL180-135, KL180-136, KL180-137, KL180-162, KL180-163, KL180-164, KL180-165, KL180-166, KL180-167, KL180-168, KL180-169, KL180-170, KL180-171, KL180-172, KL180-173, KL180-174 and analogs thereof.

In some aspects, the document provides a method of treating a CFTR-mediated diseases. The method includes administering to a subject in need thereof with a CFTR-mediated diseases one or more compounds stabilizing CFTR. The CFTR-mediated disease is associated with dysfunction of CFTR. The CFTR-mediated disease may be cystic fibrosis, male infertility, polycystic kidney disease, obstructive lung disease, intestinal obstruction syndromes, liver dysfunction, exocrine and endocrine pancreatic dysfunction, or secretory diarrhea.

In some embodiments of the disclosed methods, the compounds can be administered, e.g., orally, parenterally, intradermally, subcutaneously, topically, and/or rectally.

This disclosure additionally provides a method for identifying a bivalent compound which mediates de-ubiquitination/stabilization of AMPK, cGAS, or CFTR, the method including providing a heterobifunctional test compound including a AMPK, cGAS, or CFTR ligand conjugated to a de-ubiquitination tag (e.g. USP7 binder) via a linker, contacting the heterobifunctional test compound with a cell (e.g., a cell such as a AMPK, cGAS or CFTR-mediated disease cell) including a deubiquitinase (e.g., USP7) and AMPK, cGAS or CFTR protein.

Any of the above-described methods can further include treating the subject with one or more additional therapeutic regimens for treating the target related disease. The one or more additional therapeutic regimens for treating target related disease can be, e.g., one or more of surgery, chemotherapy, radiation therapy, hormone therapy, or immunotherapy.

As used herein, the terms “about” and “approximately” are defined as being within plus or minus 10% of a given value or state, preferably within plus or minus 5% of said value or state.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the invention will be apparent from the following detailed description and figures, and from the claims.

### BRIEF DESCRIPTION OF THE FIGURES

FIG. 1. Assessment of the effect of exemplary AMPK based bivalent compounds (XH series) on stabilization the AMPK protein level in Hela cells.

FIG. 2. Assessment of the effect of exemplary AMPK based bivalent compounds (KL series) in stabilization the AMPK protein level in Hela cells.

FIG. 3. Assessment of the effect of selected exemplary AMPK based bivalent compounds (XH series) in stabilization AMPK protein level and activating its downstream targets in in Hela cells.

FIG. 4. Assessment of the effect of selected exemplary AMPK based bivalent compounds (KL series) in stabilization AMPK protein level and activating its downstream targets in in Hela cells.

FIG. 5. Assessment of the effect of exemplary cGAS based bivalent compounds (XH series) in stabilization cGAS a protein level in Hela cells.

FIG. 6. Assessment of the effect of selected exemplary cGAS based bivalent compounds (XH series) in stabilization cGAS protein level and activating its downstream targets in Hela cells with or without DNA stimulation.

FIG. 7. Assessment of the effect of exemplary cGAS based bivalent compounds (KL series) in stabilization cGAS a protein level in Hela cells.

FIG. 8. Assessment of the effect of selected exemplary cGAS based bivalent compounds (KL series) in stabilization cGAS protein level and antiproliferation effect in Hela cells.

FIG. 9. Assessment of the effect of exemplary CFTR based bivalent compounds in stabilization CFTR protein level in human cystic fibrosis bronchial epithelial cells.

FIG. 10. Assessment of the effect of selected exemplary CFTR based bivalent compound in stabilization CFTR protein level in human cystic fibrosis bronchial epithelial cells and related mechanism of action.

### TESTING OF EXEMPLARY COMPOUNDS

The activity of novel synthesized bivalent AMPK-USP7, cGAS-USP7 and CFTR-USP7 compounds can be assessed by their ability to stabilize and activate AMPK, cGAS or CFTR proteins in related cellular assays. Assays suitable for use in any or all of these steps are known in the art, and include, e.g., Western blotting, quantitative mass spectrometry (MS) analysis, flow cytometry, enzymatic inhibition, ITC, SPR, cell growth inhibition and xenograft and PDX models.

By way of non-limiting example, detailed synthesis protocols are described in the Examples for specific exemplary of AMPK-USP7, cGAS-USP7 and CFTR-USP7 DUBTACs.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, de-acetone in place of acetone, or do-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

Pharmaceutically acceptable prodrugs of the compounds disclosed herein are contemplated and can be synthesized using conventional methods known in the art or methods corresponding to those described in the Examples (e.g., converting hydroxyl groups or carboxylic acid groups to ester groups). As used herein, a “prodrug” refers to a compound that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

### Characterization of Exemplary DUBTACs

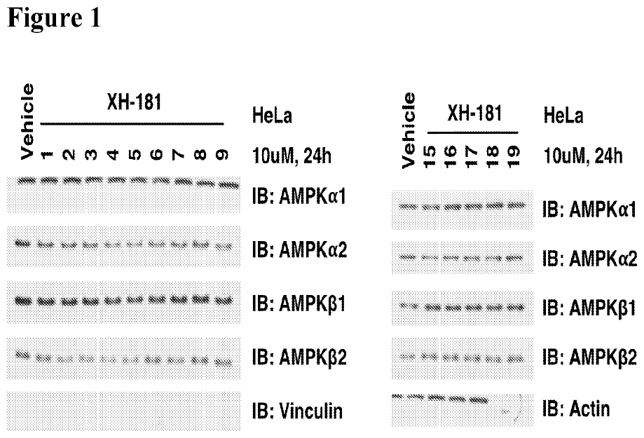

Specific exemplary DUBTACs were characterized in WB assays (Examples 168-174, FIGS. 1-7).

### Definition of Terms

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), pentyl, 3-methylhexyl, 2-methylhexyl, and the like.

“Alkylene” refers to a bivalent saturated aliphatic radical (such as ethylene) regarded as derived from an alkene by opening of the double bond or from an alkane by removal of two hydrogen atoms from different carbon atoms.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

The term “alkenylene” refers to linear or branched-chain divalent hydrocarbon radical of two to eight carbon atoms (C₂-C₈) with at least one site of unsaturation, i.e., a carbon-carbon, SP2 double bond, wherein the alkenylene radical may be optionally substituted independently with one or more substituents described herein, and includes radicals having “cis” and “trans” orientations, or alternatively, “E” and “Z” orientations. Examples include, but are not limited to, ethylenylene or vinylene (—CH═CH—), allyl (—CH₂CH═CH—), and the like.

The term “alkynylene” refers to a linear or branched divalent hydrocarbon radical of two to eight carbon atoms (C₂-C₈) with at least one site of unsaturation, i.e., a carbon-carbon, sp triple bond, wherein the alkynylene radical may be optionally substituted independently with one or more substituents described herein. Examples include, but are not limited to, ethynylene (—C≡C—), propynylene (propargylene, —CH₂C≡C—), and the like.

The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “alkoxy”, as used herein, means an alkyl group as defined herein which is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “aryl”, as used herein, “refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hückel theory. In certain embodiments, an aryl comprises six to fourteen carbon atoms (C₆-C₁₄ aryl). In certain embodiments, an aryl comprises six to ten carbon atoms (C₆-C₁₀ aryl). Examples of such groups include, but are not limited to, phenyl, fluorenyl and naphthyl. The terms “Ph” and “phenyl,” as used herein, mean a —C₆H₅ group.

The term “arylene” means a divalent aromatic hydrocarbon radical of 6-20 carbon atoms (C₆-C₂₀) derived by the removal of two hydrogen atom from a two carbon atoms of a parent aromatic ring system. Some arylene groups are represented in the exemplary structures as “Ar”. Arylene includes bicyclic radicals comprising an aromatic ring fused to a saturated, partially unsaturated ring, or aromatic carbocyclic ring. Typical arylene groups include, but not limited to, radicals derived from benzene (phenylene), substituted benzenes, naphthalene, anthracene, biphenylene indenylene, indaylene, 1,2-dihydronaphthalene, 1,2,3,4, -tetrahydronaphthyl, and the like. Arylene groups are optionally substituted with one or more substituents described herein.

The term “heteroaryl”, refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. In certain embodiments, a heteroaryl refers to a radical derived from a 3- to 10-membered aromatic ring radical (3-10 membered heteroaryl). In certain embodiments, a heteroaryl refers to a radical derived from 5- to 7-membered aromatic ring (5-7 membered heteroaryl). Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, an heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heteroalkyl”, as used herein, means a saturated or unsaturated chain of carbon atoms and at least one heteroatom, wherein no two heteroatoms are adjacent.

The term “heterocyclic”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from O, S and N, and with the proviso that the ring of said group does not contain two adjacent O atoms or two adjacent S atoms. A heterocyclic group may include fused, bridged or spirocyclic ring systems. In certain embodiments, a heterocyclic group comprises 3 to 10 ring atoms (3-10 membered heterocyclic). In certain embodiments, a heterocyclic group comprises 3 to 8 ring atoms (3-8 membered heterocyclic). In certain embodiments, a heterocyclic group comprises 4 to 8 ring atoms (4-8 membered heterocyclic). In certain embodiments, a heterocyclic group comprises 3 to 6 ring atoms (3-6 membered heterocyclic). A heterocyclic group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclic group contains a sulfur atom, said sulfur atom may be oxidized with one or two oxygen atoms to afford either a sulfoxide or sulfone. An example of a 4 membered heterocyclic group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclic groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8, diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “cycloalkyl” or “carbocyclyl” means a saturated, monocyclic, bicyclic, tricyclic, or tetracyclic radical having a total of from 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 8 carbon ring atoms (C₃-C₈ cycloalkyl). In certain embodiments, a cycloalkyl comprises 3 to 6 carbon ring atoms (C₃-C₆ cycloalkyl). Examples of such groups include, but are not limited to, cyclopropyl, cyclobutyl, cyclopentyl, cyclopentenyl, cyclohexyl, cycloheptyl, adamantyl, and the like.

The term “cycloalkylene” or “carbocyclylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C═N group.

An “aldehyde” group refers to a —C(O) H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O) OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

A “electron withdrawing” group refers to an atom or group that draws electron density from neighboring atoms towards itself.

The term “substituted,” means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O) OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “null” means the absence of an atom or moiety, and there is a bond between adjacent atoms in the structure.

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

As used herein, when m (or n or x) is defined by a range, for example, “m is 0 to 15” or “m=0-3” mean that m is an integer from 0 to 15 (i.e. m is 0, 1, 2, 3, 4, 5, 6, 7, 8, 9, 10, 11, 12, 13, 14, or 15) or m is an integer from 0 to 3 (i.e. m is 0, 1, 2, or 3) or is any integer in the defined range.

“Pharmaceutically acceptable salt” includes both acid and base addition salts. A pharmaceutically acceptable salt of any one of the bivalent compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Pharmaceutical Compositions

In some aspects, the compositions and methods described herein include the manufacture and use of pharmaceutical compositions and medicaments that include one or more bivalent compounds as disclosed herein. Also included are the pharmaceutical compositions themselves.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents known in the art. When co-administered, USP2 inhibitors disclosed herein can operate in conjunction with conventional chemotherapeutic agents to produce mechanistically additive or synergistic therapeutic effects.

In some aspects, the pH of the compositions disclosed herein can be adjusted with pharmaceutically acceptable acids, bases, or buffers to enhance the stability of the USP2 inhibitors or its delivery form.

Pharmaceutical compositions typically include a pharmaceutically acceptable carrier, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable carrier, adjuvant, or vehicle is a composition that can be administered to a patient, together with a compound of the invention, and which does not destroy the pharmacological activity thereof and is nontoxic when administered in doses sufficient to deliver a therapeutic amount of the compound. Exemplary conventional nontoxic pharmaceutically acceptable carriers, adjuvants, and vehicles include saline, solvents, dispersion media, coatings, antibacterial and antifungal agents, isotonic and absorption delaying agents, and the like, compatible with pharmaceutical administration.

In particular, pharmaceutically acceptable carriers, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

As used herein, the USP7 recruiting DUBTAC disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The USP7 recruiting DUBTAC disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivatives thereof. USP2 inhibitors disclosed herein include, e.g., those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, sulfate, salicylate, succinate, tartrate, tosylate, trifluoromethylsulfonate, and undecanoate. Salts derived from appropriate bases include, e.g., alkali metal (e.g., sodium), alkaline earth metal (e.g., magnesium), ammonium and N-(alkyl)4+ salts. The invention also envisions the quaternization of any basic nitrogen-containing groups of the USP2 inhibitors disclosed herein. Water or oil-soluble or dispersible products can be obtained by such quaternization.

In some aspects, the pharmaceutical compositions disclosed herein can include an effective amount of one or more USP7 recruiting DUBTAC. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer). In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In some aspects, the pharmaceutical compositions disclosed herein can be formulated for sale in the United States, import into the United States, or export from the United States.

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of a compound of the formulae described herein (e.g., an USP2 inhibitors) and one or more additional compounds (e.g., one or more additional compounds, drugs, or agents used for the treatment of cancer or any other condition or disease, including conditions or diseases known to be associated with or caused by cancer), both the compound and the additional compound should be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

### Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, the present disclosure provides methods for using a composition comprising a USP7 recruiting DUBTAC, including pharmaceutical compositions (indicated below as ‘X’) disclosed herein in the following methods:

Substance X for use as a medicament in the treatment of one or more diseases or conditions disclosed herein (e.g., cancer, referred to in the following examples as ‘Y’). Use of substance X for the manufacture of a medicament for the treatment of Y; and substance X for use in the treatment of Y.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of cancer.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition from which the subject is suffering (e.g., an AMPK, cGAS or CFTR-mediated disease). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the compositions and methods of the present invention. In some embodiments, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

As used herein, the term “treating cancer” means causing a partial or complete decrease in the rate of growth of a tumor, and/or in the size of the tumor and/or in the rate of local or distant tumor metastasis, and/or the overall tumor burden in a subject, and/or any decrease in tumor survival, in the presence of an bivalent compound (e.g., an USP7 recruiting DUBTAC) described herein.

As used herein, the term “virus infection” means when an organism's body is invaded by pathogenic viruses and infectious virus particle attach to and enter susceptible cells.

The terms “prevent,” “preventing,” “prevention,” “block”, and “blocking” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention. Exemplary AMPK, cGAS or CFTR-mediated diseases that can be treated with USP7 recruiting bivalent compounds include, for example, cystic fibrosis, breast cancer, ovarian cancer, prostate cancer, colon cancer, pancreatic cancer, bladder cancer, liver cancer and cervical cancer.

As used herein, the term “preventing a disease” (e.g., preventing cancer) in a subject means for example, to stop the development of one or more symptoms of a disease in a subject before they occur or are detectable, e.g., by the patient or the patient's doctor. Preferably, the disease (e.g., cancer) does not develop at all, i.e., no symptoms of the disease are detectable. However, it can also result in delaying or slowing of the development of one or more symptoms of the disease. Alternatively, or in addition, it can result in the decreasing of the severity of one or more subsequently developed symptoms.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

The present disclosure is also described and demonstrated by way of the following examples. However, the use of these and other examples anywhere in the specification is illustrative only and in no way limits the scope and meaning of the invention or of any exemplified term. Likewise, the invention is not limited to any particular preferred embodiment or aspect described herein. Indeed, many modifications and variations may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Chemistry General Procedures

All commercial chemical reagents and solvents were used for the reactions without further purification. Flash column chromatography was performed on Teledyne ISCO CombiFlash Rf+ instrument equipped with a 220/254/280 nm wavelength UV detector and a fraction collector. Normal phase column chromatography was conducted on silica gel columns with either hexane/ethyl acetate or dichloromethane/methanol as eluent. Reverse phase column chromatography was conducted on HP C18 RediSep Rf columns, and the gradient was set to 10% of acetonitrile in H₂O containing 0.1% TFA progressing to 100% of acetonitrile. All final compounds were purified with preparative high-performance liquid chromatography (HPLC) on an Agilent Prep 1200 series with the UV detector set to 220/254 nm at a flow rate of 40 mL/min. Samples were injected onto a Phenomenex Luna 750×30 mm, 5 μm C18 column, and the gradient was set to 10% of acetonitrile in H₂O containing 0.1% TFA progressing to 100% of acetonitrile. All compounds assessed for biological activity have purity >95% as determined by an Agilent 1200 series system with DAD detector and a 2.1 mm×150 mm Zorbax 300SB—C18 5 μm column for chromatography and high-resolution mass spectra (HRMS) that were acquired in positive ion mode using an Agilent G1969A API-TOF with an electrospray ionization (ESI) source. Samples (2 μL) were injected onto a C18 column at room temperature, and the flow rate was set to 0.4 mL/min with water containing 0.1% formic acid as solvent A and acetonitrile containing 0.1% formic acid as solvent B. Nuclear magnetic resonance (NMR) spectra were acquired on Bruker DRX 400 MHz for proton (¹H NMR) and 133 MHz for carbon (13C NMR). Chemical shifts for all compounds are reported in parts per million (ppm, δ). The format of chemical shift was reported as follows: chemical shift, multiplicity (s=singlet, d=doublet, t=triplet, q=quartet, m=multiplet), coupling constant (J values in Hz), and integration. All final compounds had >95% purity using the HPLC methods described above.

### EXAMPLES

### Procedure for the Synthesis of AMPK Based Bivalent Compounds

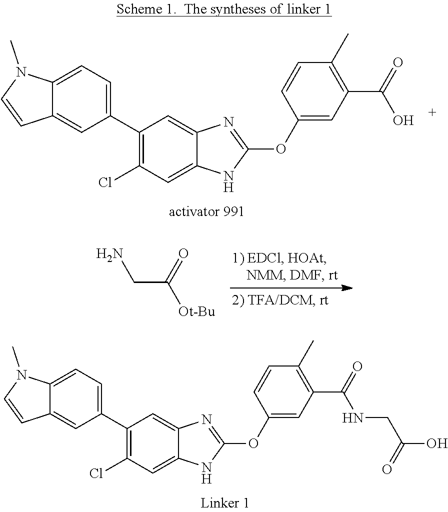 [SMILES: Cc1ccc(Oc2nc3cc(-c4ccc5c(ccn5C)c4)c(Cl)cc3[nH]2)cc1C(=O)O ; CC(C)(C)OC(=O)CN ; Cc1ccc(Oc2nc3cc(-c4ccc5c(ccn5C)c4)c(Cl)cc3[nH]2)cc1C(=O)NCC(=O)O]

### Linker 1: (5-((6-chloro-5-(1-methyl-1H-indol-5-yl)-1H-benzo[d]imidazol-2-yl)oxy)-2-methylbenzoyl)glycine

To a solution of activator 991 (prepared according to previous published paper) (Ngoei et al., 2018) (50 mg, 0.12 mmol) in DMF (1 mL) were added tert-butyl(2-aminoethyl)carbamate (19.2 mg, 0.12 mmol, 1.0 equiv), 1-ethyl-3-(3-dimethylaminopropyl)carbodiimide (EDCI, 34.3 mg, 0.18 mmol, 1.5 equiv), 1-hydroxy-7-azabenzo-triazole (HOAt, 24.5 mg, 0.18 mmol, 1.5 equiv), and N-methylmorpholine (NMM, 36.4 mg, 0.36 mmol, 3.0 equiv). After being stirred for overnight at rt, the resulting mixture was purified by prep-HPLC to afford crude product. The crude product was dissolved in DCM (1 mL) and TFA (1 mL). After being stirred at rt for 30 min, the resulting mixture was purified by prep-HPLC to afford compound titled compound as yellow solid (57 mg, yield 81%). MS (ESI) [M+H]⁺=489.3. Linker 2-13 were synthesized following the same procedure for preparing linker 1

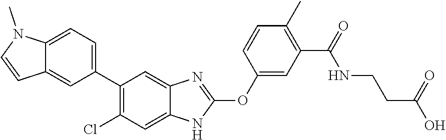 [SMILES: Cc1ccc(Oc2nc3cc(-c4ccc5c(ccn5C)c4)c(Cl)cc3[nH]2)cc1C(=O)NCCC(=O)O]

### Linker 2: 3-(5-((6-chloro-5-(1-methyl-1H-indol-5-yl)-1H-benzo[d]imidazol-2-yl)oxy)-2-methylbenzamido)propanoic acid

MS (ESI) [M+H]⁺=503.2.

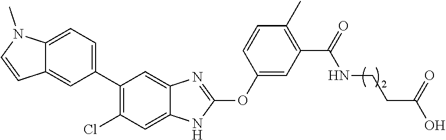

### Linker 3: 4-(5-((6-chloro-5-(1-methyl-1H-indol-5-yl)-1H-benzo[d]imidazol-2-yl)oxy)-2-methylbenzamido)butanoic acid

MS (ESI) [M+H]⁺=517.4.

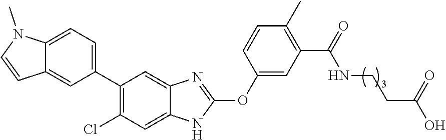

### Linker 4: 5-(5-((6-chloro-5-(1-methyl-1H-indol-5-yl)-1H-benzo[d]imidazol-2-yl)oxy)-2-methylbenzamido)pentanoic acid

MS (ESI) [M+H]⁺=531.3.

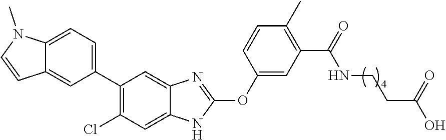

### Linker 5: 6-(5-((6-chloro-5-(1-methyl-1H-indol-5-yl)-1H-benzo[d]imidazol-2-yl)oxy)-2-methylbenzamido)hexanoic acid

MS (ESI) [M+H]⁺=545.3.

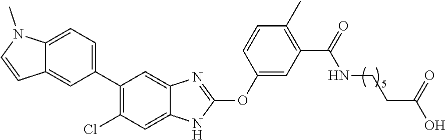

### Linker 6: 7-(5-((6-chloro-5-(1-methyl-1H-indol-5-yl)-1H-benzo[d]imidazol-2-yl)oxy)-2-methylbenzamido)heptanoic acid

MS (ESI) [M+H]⁺=559.4.

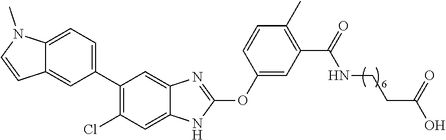

### Linker 7: 8-(5-((6-chloro-5-(1-methyl-1H-indol-5-yl)-1H-benzo[d]imidazol-2-yl)oxy)-2-methylbenzamido)octanoic acid

MS (ESI) [M+H]⁺=573.3.

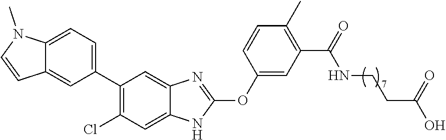

### Linker 8: 9-(5-((6-chloro-5-(1-methyl-1H-indol-5-yl)-1H-benzo[d]imidazol-2-yl)oxy)-2-methylbenzamido)nonanoic acid

MS (ESI) [M+H]⁺=587.5.

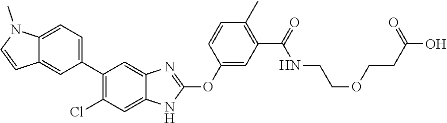 [SMILES: Cc1ccc(Oc2nc3cc(-c4ccc5c(ccn5C)c4)c(Cl)cc3[nH]2)cc1C(=O)NCCOCCC(=O)O]

### Linker 9: 3-(2-(5-((6-chloro-5-(1-methyl-1H-indol-5-yl)-1H-benzo[d]imidazol-2-yl)oxy)-2-methylbenzamido)ethoxy)propanoic acid

MS (ESI) [M+H]⁺=548.6.

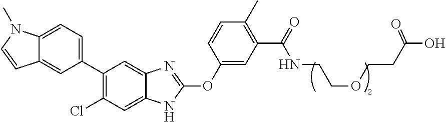

### Linker 10: 3-(2-(2-(5-((6-chloro-5-(1-methyl-1H-indol-5-yl)-1H-benzo[d]imidazol-2-yl)oxy)-2-methylbenzamido)ethoxy)ethoxy)propanoic acid

MS (ESI) [M+H]⁺=591.4.

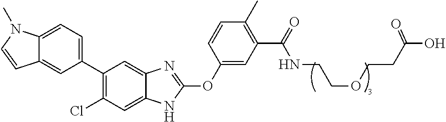

### Linker 11: 1-(5-((6-chloro-5-(1-methyl-1H-indol-5-yl)-1H-benzo[d]imidazol-2-yl)oxy)-2-methylphenyl)-1-oxo-5,8,11-trioxa-2-azatetradecan-14-oic acid

MS (ESI) [M+H]⁺=635.4.

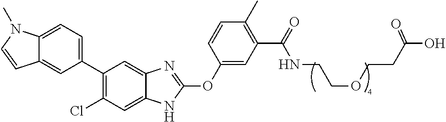

### Linker 12: 1-(5-((6-chloro-5-(1-methyl-1H-indol-5-yl)-1H-benzo[d]imidazol-2-yl)oxy)-2-methylphenyl)-1-oxo-5,8,11,14-tetraoxa-2-azaheptadecan-17-oic acid

MS (ESI) [M+H]⁺=679.4.

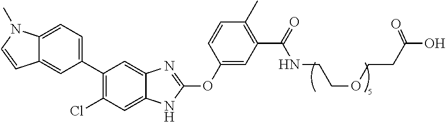

### Linker 13: 1-(5-((6-chloro-5-(1-methyl-1H-indol-5-yl)-1H-benzo[d]imidazol-2-yl)oxy)-2-methylphenyl)-1-oxo-5,8,11,14,17-pentaoxa-2-azaicosan-20-oic acid

MS (ESI) [M+H]⁺=723.3.

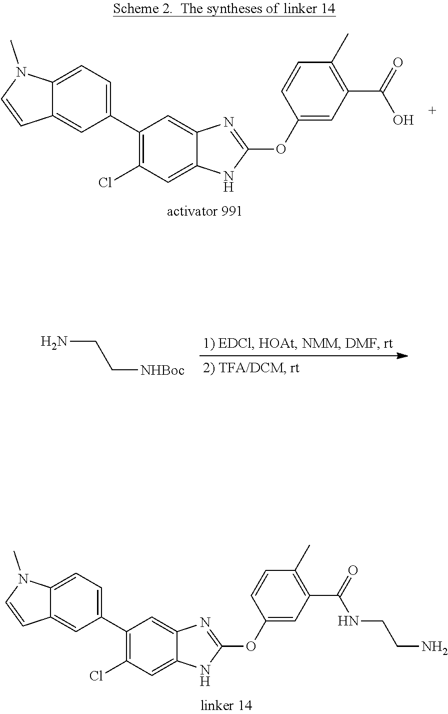 [SMILES: Cc1ccc(Oc2nc3cc(-c4ccc5c(ccn5C)c4)c(Cl)cc3[nH]2)cc1C(=O)O ; CC(C)(C)OC(=O)NCCN ; Cc1ccc(Oc2nc3cc(-c4ccc5c(ccn5C)c4)c(Cl)cc3[nH]2)cc1C(=O)NCCN]

### Linker 14: N-(2-aminoethyl)-5-((6-chloro-5-(1-methyl-1H-indol-5-yl)-1H-benzo[d]imidazol-2-yl)oxy)-2-methylbenzamide

To a solution of activator 991 (50 mg, 0.12 mmol) in DMF (1 mL) were added tert-butyl(2-aminoethyl)carbamate (19.2 mg, 0.12 mmol, 1.0 equiv), 1-ethyl-3-(3-dimethylaminopropyl)carbodiimide (EDCI, 34.3 mg, 0.18 mmol, 1.5 equiv), 1-hydroxy-7-azabenzo-triazole (HOAt, 24.5 mg, 0.18 mmol, 1.5 equiv), and N-methylmorpholine (NMM, 36.4 mg, 0.36 mmol, 3.0 equiv). After being stirred for overnight at rt, the resulting mixture was purified by preparative HPLC (10%-100% acetonitrile/0.1% TFA in H₂O) to afford crude product. The crude product was dissolved in DCM (1 mL) and TFA (1 mL). After being stirred at rt for 30 min, the resulting mixture was purified by preparative HPLC (10%-100% acetonitrile/0.1% TFA in H₂O) to afford compound titled compound as yellow solid (57 mg, yield 81%). MS (ESI) [M+H]⁺=474.4.

Linker 15-27 was synthesized following the same procedure for preparing linker 14.

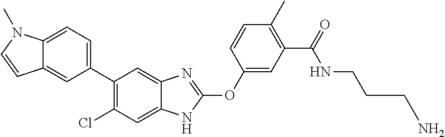 [SMILES: Cc1ccc(Oc2nc3cc(-c4ccc5c(ccn5C)c4)c(Cl)cc3[nH]2)cc1C(=O)NCCCN]

### Linker 15: N-(3-aminopropyl)-5-((6-chloro-5-(1-methyl-1H-indol-5-yl)-1H-benzo[d]imidazol-2-yl)oxy)-2-methylbenzamide (XH168-165)

Pink solid, 52 mg, 61% yield. MS (ESI) [M+H]⁺=488.3.

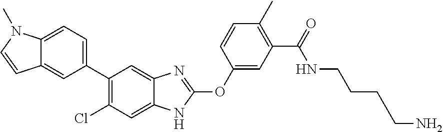 [SMILES: Cc1ccc(Oc2nc3cc(-c4ccc5c(ccn5C)c4)c(Cl)cc3[nH]2)cc1C(=O)NCCCCN]

### Linker 16: N-(4-aminobutyl)-5-((6-chloro-5-(1-methyl-1H-indol-5-yl)-1H-benzo[d]imidazol-2-yl)oxy)-2-methylbenzamide (XH168-166)

Pink solid, 59 mg, 67% yield. MS (ESI) [M+H]⁺=502.4.

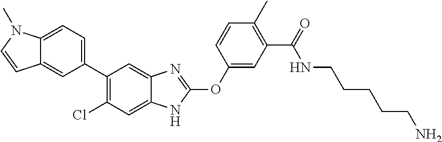 [SMILES: Cc1ccc(Oc2nc3cc(-c4ccc5c(ccn5C)c4)c(Cl)cc3[nH]2)cc1C(=O)NCCCCCN]

### Linker 17: N-(5-aminopentyl)-5-((6-chloro-5-(1-methyl-1H-indol-5-yl)-1H-benzo[d]imidazol-2-yl)oxy)-2-methylbenzamide (XH168-167)

Yellow solid, 64 mg, 72% yield. MS (ESI) [M+H]⁺=516.9.

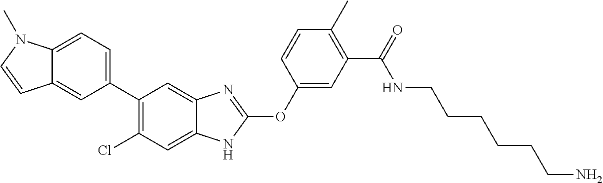 [SMILES: Cc1ccc(Oc2nc3cc(-c4ccc5c(ccn5C)c4)c(Cl)cc3[nH]2)cc1C(=O)NCCCCCCN]

### Linker 18: N-(6-aminohexyl)-5-((6-chloro-5-(1-methyl-1H-indol-5-yl)-1H-benzo[d]imidazol-2-yl)oxy)-2-methylbenzamide (XH168-168)

Yellow solid, 72 mg, 79% yield. MS (ESI) [M+H]⁺=530.1.

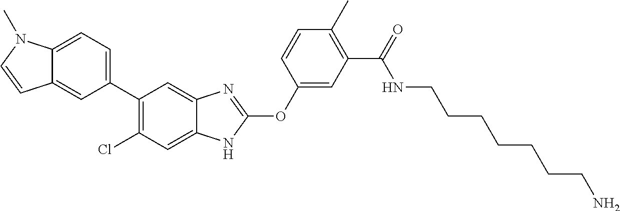 [SMILES: Cc1ccc(Oc2nc3cc(-c4ccc5c(ccn5C)c4)c(Cl)cc3[nH]2)cc1C(=O)NCCCCCCCN]

### Linker 19: N-(7-aminoheptyl)-5-((6-chloro-5-(1-methyl-1H-indol-5-yl)-1H-benzo[d]imidazol-2-yl)oxy)-2-methylbenzamide (XH168-169)

Yellow solid, 73 mg, 79% yield. MS (ESI) [M+H]⁺=544.5.

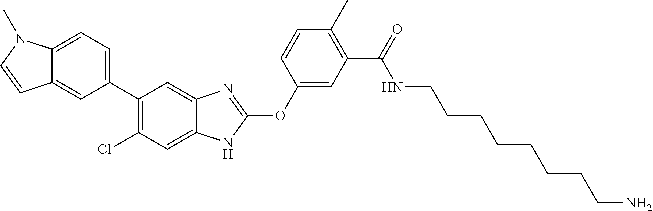 [SMILES: Cc1ccc(Oc2nc3cc(-c4ccc5c(ccn5C)c4)c(Cl)cc3[nH]2)cc1C(=O)NCCCCCCCCN]

### Linker 20: N-(8-aminooctyl)-5-((6-chloro-5-(1-methyl-1H-indol-5-yl)-1H-benzo[d]imidazol-2-yl)oxy)-2-methylbenzamide (XH168-170)

Yellow solid, 69 mg, 73% yield. MS (ESI) [M+H]⁺=558.4.

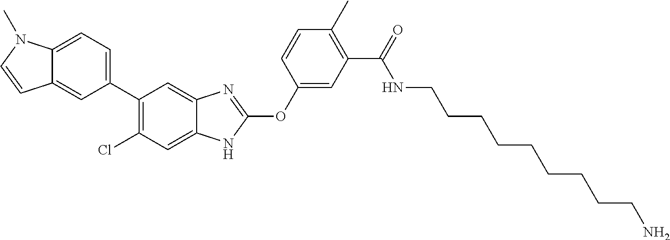 [SMILES: Cc1ccc(Oc2nc3cc(-c4ccc5c(ccn5C)c4)c(Cl)cc3[nH]2)cc1C(=O)NCCCCCCCCCN]

### Linker 21: N-(9-aminononyl)-5-((6-chloro-5-(1-methyl-1H-indol-5-yl)-1H-benzo[d]imidazol-2-yl)oxy)-2-methylbenzamide (XH168-171)

Yellow solid, 78 mg, 81% yield. MS (ESI) [M+H]⁺=572.4.

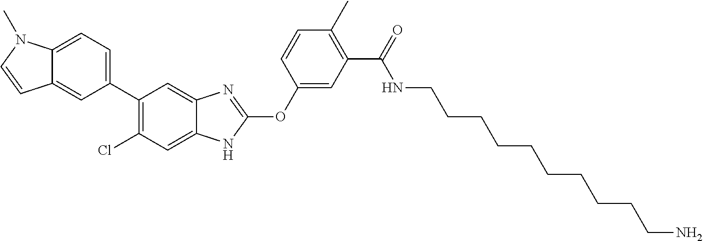 [SMILES: Cc1ccc(Oc2nc3cc(-c4ccc5c(ccn5C)c4)c(Cl)cc3[nH]2)cc1C(=O)NCCCCCCCCCCN]

### Linker 22: N-(10-aminodecyl)-5-((6-chloro-5-(1-methyl-1H-indol-5-yl)-1H-benzo[d]imidazol-2-yl)oxy)-2-methylbenzamide (XH168-172)

Yellow solid, 75 mg, 87% yield. MS (ESI) [M+H]⁺=586.5.

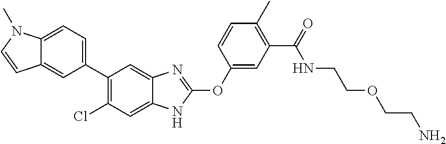 [SMILES: Cc1ccc(Oc2nc3cc(-c4ccc5c(ccn5C)c4)c(Cl)cc3[nH]2)cc1C(=O)NCCOCCN]

### Linker 23: N-(2-(2-aminoethoxy)ethyl)-5-((6-chloro-5-(1-methyl-1H-indol-5-yl)-1H-benzo[d]imidazol-2-yl)oxy)-2-methylbenzamide (XH168-173)

Yellow solid, 65 mg, 73% yield. MS (ESI) [M+H]⁺=518.1.

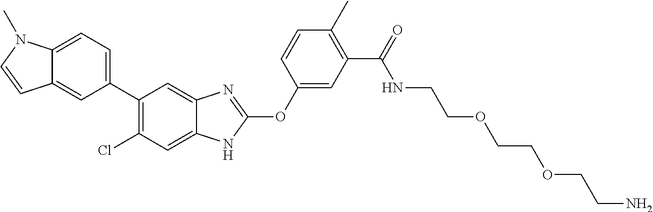 [SMILES: Cc1ccc(Oc2nc3cc(-c4ccc5c(ccn5C)c4)c(Cl)cc3[nH]2)cc1C(=O)NCCOCCOCCN]

### Linker 24: N-(2-(2-(2-aminoethoxy)ethoxy)ethyl)-5-((6-chloro-5-(1-methyl-1H-indol-5-yl)-1H-benzo[d]imidazol-2-yl)oxy)-2-methylbenzamide (XH168-174)

Yellow solid, 79 mg, 81% yield. MS (ESI) [M+H]⁺=562.5.

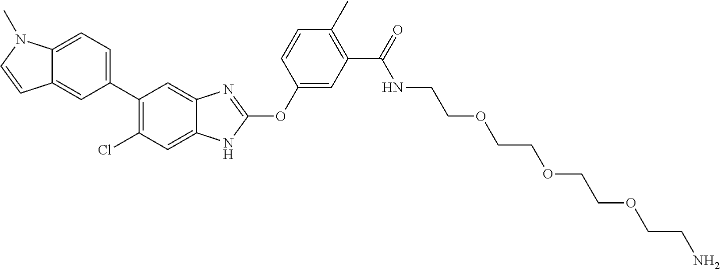 [SMILES: Cc1ccc(Oc2nc3cc(-c4ccc5c(ccn5C)c4)c(Cl)cc3[nH]2)cc1C(=O)NCCOCCOCCOCCN]

### Linker 25: N-(2-(2-(2-(2-aminoethoxy)ethoxy)ethoxy)ethyl)-5-((6-chloro-5-(1-methyl-1H-indol-5-yl)-1H-benzo[d]imidazol-2-yl)oxy)-2-methylbenzamide (XH168-175)

Yellow solid, 76 mg, 76% yield. MS (ESI) [M+H]⁺=606.3.

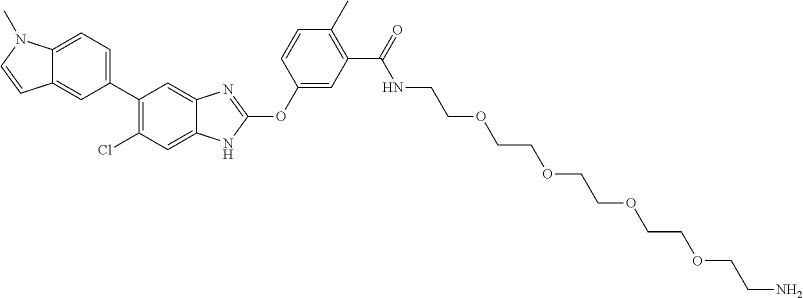 [SMILES: Cc1ccc(Oc2nc3cc(-c4ccc5c(ccn5C)c4)c(Cl)cc3[nH]2)cc1C(=O)NCCOCCOCCOCCOCCN]

### Linker 26: N-(14-amino-3,6,9,12-tetraoxatetradecyl)-5-((6-chloro-5-(1-methyl-1H-indol-5-yl)-1H-benzo[d]imidazol-2-yl)oxy)-2-methylbenzamide (XH168-176)

Yellow solid, 86 mg, 81% yield. MS (ESI) [M+H]⁺=650.2.

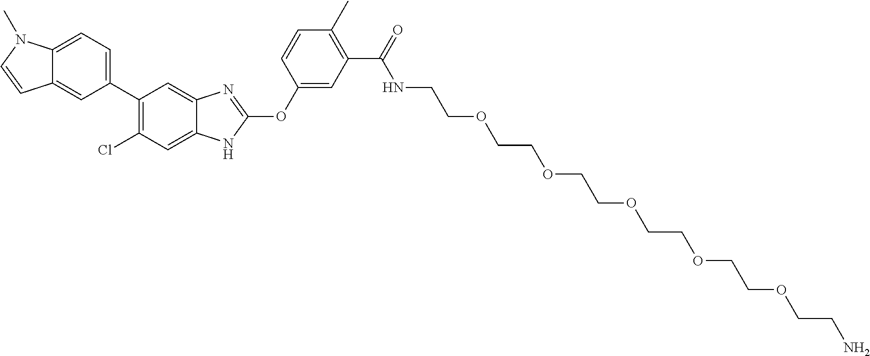 [SMILES: Cc1ccc(Oc2nc3cc(-c4ccc5c(ccn5C)c4)c(Cl)cc3[nH]2)cc1C(=O)NCCOCCOCCOCCOCCOCCN]

### Linker 27: N-(17-amino-3,6,9,12,15-pentaoxaheptadecyl)-5-((6-chloro-5-(1-methyl-1H-indol-5-yl)-1H-benzo[d]imidazol-2-yl)oxy)-2-methylbenzamide (XH168-177)

Yellow solid, 89 mg, 81% yield. MS (ESI) [M+H]⁺=694.5.

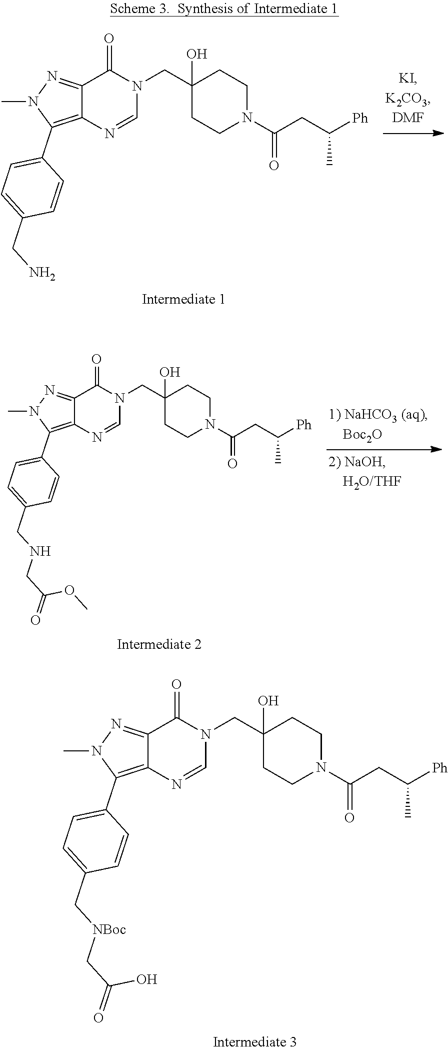 [SMILES: C[C@H](CC(=O)N1CCC(O)(Cn2cnc3c(-c4ccc(CN)cc4)n(C)nc3c2=O)CC1)c1ccccc1 ; COC(=O)CNCc1ccc(-c2c3ncn(CC4(O)CCN(C(=O)C[C@@H](C)c5ccccc5)CC4)c(=O)c3nn2C)cc1 ; C[C@H](CC(=O)N1CCC(O)(Cn2cnc3c(-c4ccc(CN(CC(=O)O)C(=O)OC(C)(C)C)cc4)n(C)nc3c2=O)CC1)c1ccccc1]

### Intermediate 3: (R)—N-(tert-butoxycarbonyl)-N-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)glycine (XH161-1)

To a solution of intermediate 1 (prepared according to previous report) (Gavory et al., 2018) (605.4 mg, 1.2 mmol) in DMF (5 mL) were added methyl 2-bromoacetate (183.6 mg, 1.2 mmol, 1.0 equiv), KI (398.4 mg, 2.4 mmol, 2 equiv), K₂CO₃ (331.8 mg, 2.4 mmol, 2 equiv). After being stirred for 3 hours at rt, the resulting mixture was purified by reversed ISCO to afford intermediate 2. Intermediate 2 was dissolved in THF/H₂O (2:1, 6 mL), sat. NaHCO₃ aqueous solution (2 mL) and di-tert-butyl decarbonate ((Boc)₂O, 139 mg, 0.637 mmol, 1.3 equiv). And the reaction was stirred at rt for 3 h. Then, the solvent was evaporated, the resulting mixture was purified by reversed ISCO to afford crude product. To the obtained crude product in THF/H₂O (1:1, 4 mL) was added lithium hydroxide (LiOH, 23.5 mg, 0.9 mmol, 2 equiv). After being stirred at rt for 1 h, the resulting mixture was purified by reversed ISCO to afford titled compound as white solid (250 mg, yield 76% for three steps). ¹H NMR (400 MHz, Methanol-d₄) δ 8.00-7.95 (m, 1H), 7.65-7.61 (m, 2H), 7.51-7.49 (m, 2H), 7.33-7.13 (m, 5H), 4.60 (s, 2H), 4.24-4.20 (m, 1H), 4.15-4.10 (m, 3H), 4.05-3.95 (m, 2H), 3.90-3.87 (m, 2H), 3.70-3.63 (m, 1H), 3.27-3.14 (m, 2H), 3.05-2.89 (m, 1H), 2.82-2.70 (m, 1H), 2.62-2.45 (m, 1H), 1.65-1.57 (m, 1H), 1.51-1.46 (m, 9H), 1.44-1.39 (m, 1H), 1.37-1.28 (m, 4H), 0.94-0.86 (m, 1H). MS (ESI) [M+H]⁺=673.85.

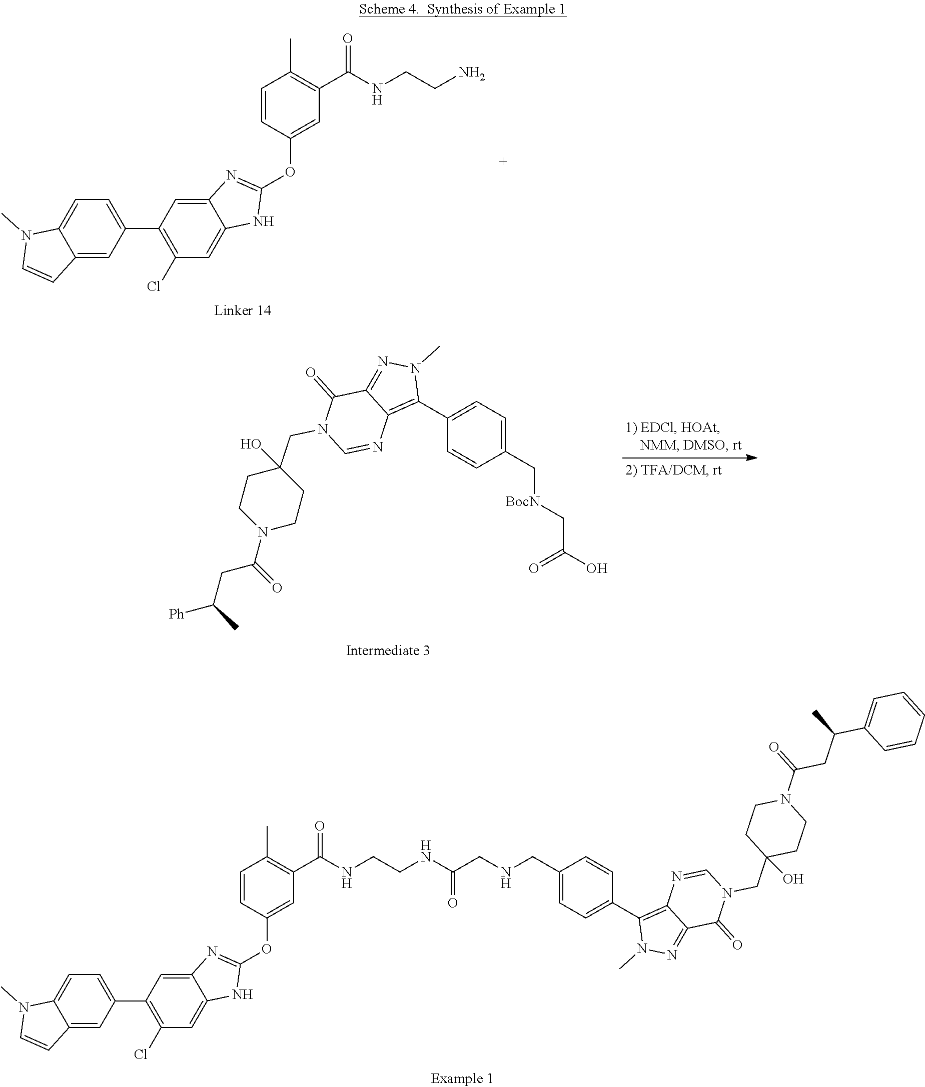 [SMILES: Cc1ccc(Oc2nc3cc(-c4ccc5c(ccn5C)c4)c(Cl)cc3[nH]2)cc1C(=O)NCCN ; C[C@H](CC(=O)N1CCC(O)(Cn2cnc3c(-c4ccc(CN(CC(=O)O)C(=O)OC(C)(C)C)cc4)n(C)nc3c2=O)CC1)c1ccccc1 ; Cc1ccc(Oc2nc3cc(-c4ccc5c(ccn5C)c4)c(Cl)cc3[nH]2)cc1C(=O)NCCNC(=O)CNCc1ccc(-c2c3ncn(CC4(O)CCN(C(=O)C[C@@H](C)c5ccccc5)CC4)c(=O)c3nn2C)cc1]

### Example 1: (R)-5-((6-chloro-5-(1-methyl-1H-indol-5-yl)-1H-benzo[d]imidazol-2-yl)oxy)-N-(2-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)ethyl)-2-methylbenzamide (XH181-1)

To a solution of linker 14 (10.5 mg, 0.015 mmol) in DMF (0.5 mL) were added intermediate 3 (10 mg, 0.015 mmol, 1.0 equiv), 1-ethyl-3-(3-dimethylaminopropyl)carbodiimide (EDCI, 4.3 mg, 0.0225 mmol, 1.5 equiv), 1-hydroxy-7-azabenzo-triazole (HOAt, 3.1 mg, 0.0225 mmol, 1.5 equiv), and N-methylmorpholine (NMM, 4.5 mg, 0.045 mmol, 3.0 equiv). After being stirred overnight at rt, the resulting mixture was purified by preparative HPLC to afford crude product. The crude product was re-dissolved in DCM/TFA (1:1, 1 mL) followed by stirred at rt for 30 min. The resulting mixture was purified by preparative HPLC to afford titled compound as pink solid (5.6 mg, yield 30%). ¹H NMR (400 MHz, Methanol-d₄) δ 7.97-7.85 (m, 1H), 7.65-7.42 (m, 7H), 7.39-7.11 (m, 12H), 4.29-4.17 (m, 3H), 4.05-3.90 (m, 5H), 3.84-3.76 (m, 5H), 3.69-3.49 (m, 6H), 3.25-3.14 (m, 2H), 3.03-2.86 (m, 1H), 2.80-2.54 (m, 2H), 2.44-2.43 (m, 3H), 1.61-1.52 (m, 1H), 1.40-1.25 (m, 6H). MS (ESI) [M+H]⁺=1028.9.

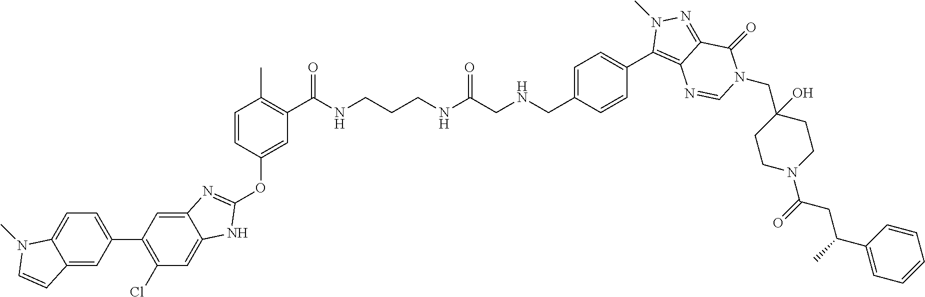 [SMILES: Cc1ccc(Oc2nc3cc(-c4ccc5c(ccn5C)c4)c(Cl)cc3[nH]2)cc1C(=O)NCCCNC(=O)CNCc1ccc(-c2c3ncn(CC4(O)CCN(C(=O)C[C@@H](C)c5ccccc5)CC4)c(=O)c3nn2C)cc1]

### Example 2: (R)-5-((6-chloro-5-(1-methyl-1H-indol-5-yl)-1H-benzo[d]imidazol-2-yl)oxy)-N-(3-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)propyl)-2-methylbenzamide (Xh181-2)

Example 2 was synthesized following the same procedure for preparing example 1 from linker 15. White solid, 4.7 mg, yield 25%, ¹H NMR (400 MHz, Methanol-d₄) δ 7.99-7.84 (m, 1H), 7.73-7.55 (m, 4H), 7.51-7.11 (m 15H), 4.31-4.13 (m, 3H), 4.09-3.91 (m, 5H), 3.84-3.73 (m, 5H), 3.63-3.59 (m, 1H), 3.47-3.38 (m, 5H), 3.26-3.12 (m, 2H), 3.02-2.81 (m, 1H), 2.75-2.70 (m, 2H), 2.43-2.41 (m, 3H), 1.82 (s, 2H), 1.57-1.54 (m, 1H), 1.39-1.28 (m, 6H).). MS (ESI) [M+H]⁻=1042.5.

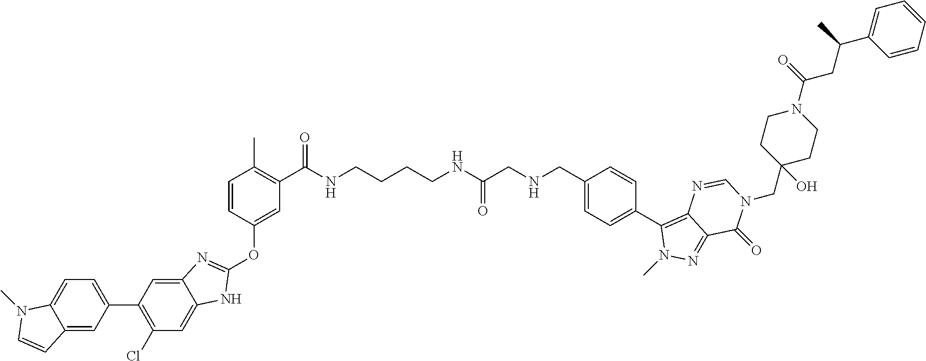 [SMILES: Cc1ccc(Oc2nc3cc(-c4ccc5c(ccn5C)c4)c(Cl)cc3[nH]2)cc1C(=O)NCCCCNC(=O)CNCc1ccc(-c2c3ncn(CC4(O)CCN(C(=O)C[C@@H](C)c5ccccc5)CC4)c(=O)c3nn2C)cc1]

### Example 3: (R)-5-((6-chloro-5-(1-methyl-1H-indol-5-yl)-1H-benzo[d]imidazol-2-yl)oxy)-N-(4-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)butyl)-2-methylbenzamide (Xh181-3)

Example 3 was synthesized following the same procedure for preparing example 1 from intermediate 3 and linker 16. White solid, 2.3 mg, yield 12%, ¹H NMR (400 MHz, DMSO-d₆) δ 9.31 (s, 2H), 8.39 (d, J=20.0 Hz, 2H), 7.99 (d, J=12.0 Hz, 1H), 7.80-7.78 (m, 2H), 7.68-7.66 (m, 2H), 7.54-7.46 (m, 2H), 7.37-7.24 (m, 8H), 7.18-7.14 (d, J=6.7 Hz, 2H), 4.87 (s, 1H), 4.29-4.25 (m, 2H), 4.11-3.90 (m, 6H), 3.83-3.68 (m, 4H), 3.23-3.16 (m, 6H), 2.90-2.85 (m, 1H), 2.69-2.56 (m, 2H), 2.35-2.33 (m, 3H), 1.50 (s, 4H), 1.38-1.15 (m, 7H). MS (ESI) [M+H]⁺=1056.8.

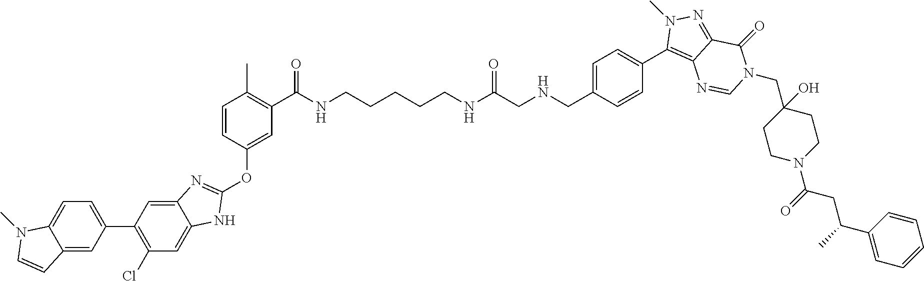 [SMILES: Cc1ccc(Oc2nc3cc(-c4ccc5c(ccn5C)c4)c(Cl)cc3[nH]2)cc1C(=O)NCCCCCNC(=O)CNCc1ccc(-c2c3ncn(CC4(O)CCN(C(=O)C[C@@H](C)c5ccccc5)CC4)c(=O)c3nn2C)cc1]

### Example 4: (R)-5-((6-chloro-5-(1-methyl-1H-indol-5-yl)-1H-benzo[d]imidazol-2-yl)oxy)-N-(5-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)pentyl)-2-methylbenzamide (Xh181-4)

Example 4 was synthesized following the same procedure for preparing example 1 from intermediate 3 and linker 17. Pink solid, 3.0 mg, yield 15%, ¹H NMR (400 MHz, DMSO-d₆) δ 9.32 (s, 2H), 8.41-8.38 (m, 2H), 7.99 (d, J=8.0 Hz, 1H), 7.79 (d, J=8.0 Hz, 2H), 7.67 (d, J=8.0 Hz, 2H), 7.54-7.45 (m, 2H), 7.37-7.24 (m, 8H), 7.18-7.16 (m, 2H), 4.25 (s, 3H), 4.12-4.11 (m, 5H), 4.05-3.99 (m, 7H), 3.82-3.62 (m, 5H), 3.20-3.12 (m, 7H), 2.91-2.85 (m, 1H), 2.67-2.57 (m, 2H), 2.34 (s, 3H), 1.51-1.19 (m, 13H). MS (ESI) [M+H]⁺=1070.3.

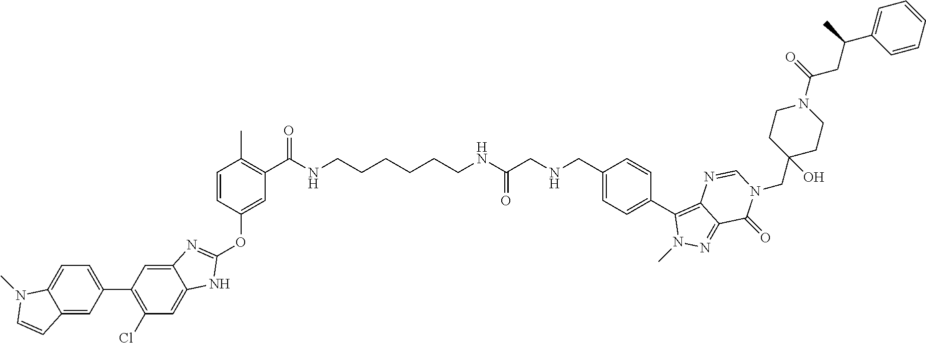 [SMILES: Cc1ccc(Oc2nc3cc(-c4ccc5c(ccn5C)c4)c(Cl)cc3[nH]2)cc1C(=O)NCCCCCCNC(=O)CNCc1ccc(-c2c3ncn(CC4(O)CCN(C(=O)C[C@@H](C)c5ccccc5)CC4)c(=O)c3nn2C)cc1]

### Example 5: (R)-5-((6-chloro-5-(1-methyl-1H-indol-5-yl)-1H-benzo[d]imidazol-2-yl)oxy)-N-(6-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)hexyl)-2-methylbenzamide (XH181-5)

Example 5 was synthesized following the same procedure for preparing example 1 from intermediate 3 and linker 18. White solid, 1.5 mg, yield 8%, ¹H NMR (400 MHz, DMSO-d₆) δ 9.31 (s, 2H), 8.38-8.34 (m, 2H), 7.99 (d, J=8.0 Hz, 1H), 7.79 (d, J=8.0 Hz, 2H), 7.72-7.61 (m, 2H), 7.54-7.45 (m, 2H), 7.36-7.25 (m, 8H), 7.43-7.21 (m, 8H), 7.19-7.15 (m, 2H), 4.29-4.25 (s, 2H), 4.11 (s, 3H), 4.00-3.94 (m, 8H), 3.21-3.12 (m, 7H), 2.91-2.85 (m, 1H), 2.68-2.56 (m, 2H), 2.33 (s, 3H), 1.52-1.19 (m, 15H). MS (ESI) [M+H]⁺=1084.7.

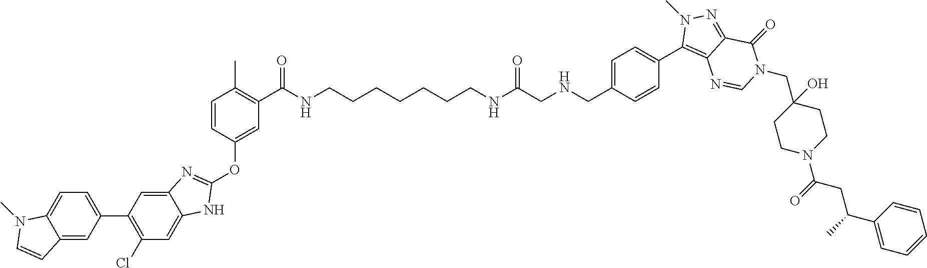 [SMILES: Cc1ccc(Oc2nc3cc(-c4ccc5c(ccn5C)c4)c(Cl)cc3[nH]2)cc1C(=O)NCCCCCCCNC(=O)CNCc1ccc(-c2c3ncn(CC4(O)CCN(C(=O)C[C@@H](C)c5ccccc5)CC4)c(=O)c3nn2C)cc1]

### Example 6: (R)-5-((6-chloro-5-(1-methyl-1H-indol-5-yl)-1H-benzo[d]imidazol-2-yl)oxy)-N-(7-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)heptyl)-2-methylbenzamide (XH181-6)

Example 6 was synthesized following the same procedure for preparing example 1 from intermediate 3 and linker 19. White solid, 1.9 mg, yield 9%, ¹H NMR (400 MHz, DMSO-d₆) δ 9.30 (s, 2H), 8.37-8.33 (m, 2H), 8.00-7.97 (m, 1H), 7.79 (d, J=8.0 Hz, 2H), 7.72-7.60 (m, 3H), 7.53-7.46 (m, 2H), 7.38-7.23 (m, 8H), 7.17-7.15 (m, 2H), 4.84 (s, 1H), 4.29-4.25 (m, 2H), 4.11 (s, 3H), 4.03-3.82 (m, 8H), 3.19-3.10 (m, 7H), 2.91-2.85 (m, 1H), 2.68-2.56 (m, 2H), 2.33 (s, 3H), 1.55-1.19 (m, 17H). MS (ESI) [M+H]⁺=1099.4.

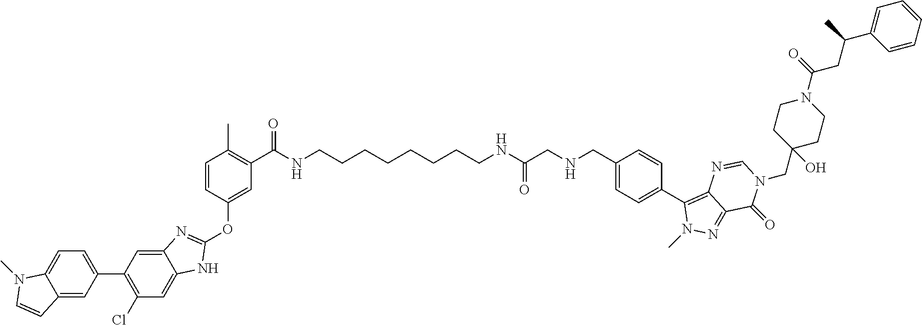 [SMILES: Cc1ccc(Oc2nc3cc(-c4ccc5c(ccn5C)c4)c(Cl)cc3[nH]2)cc1C(=O)NCCCCCCCCNC(=O)CNCc1ccc(-c2c3ncn(CC4(O)CCN(C(=O)C[C@@H](C)c5ccccc5)CC4)c(=O)c3nn2C)cc1]

### Example 7: (R)-5-((6-chloro-5-(1-methyl-1H-indol-5-yl)-1H-benzo[d]imidazol-2-yl)oxy)-N-(8-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)octyl)-2-methylbenzamide (XH181-7)

XH181-7 was synthesized following the same procedure for preparing example 1 from intermediate 3 and linker 20. Pink solid, 2.0 mg, yield 10%, ¹H NMR (400 MHz, DMSO-d₆) δ 9.43 (s, 2H), 8.42-8.34 (m, 2H), 8.01 (d, J=12.0 Hz, 1H), 7.78 (d, J=8.0 Hz, 2H), 7.69 (d, J=8.0 Hz, 2H), 7.63-7.45 (m, 2H), 7.39-7.24 (m, 8H), 7.19-7.15 (m, 2H), 4.84 (s, 1H), 4.29-4.25 (s, 2H), 4.12 (s, 3H), 4.04-3.94 (m, 8H), 3.25-3.10 (m, 7H), 2.90-2.84 (m, 1H), 2.67-2.58 (m, 2H), 2.33 (s, 3H), 1.49-1.19 (m, 19H). MS (ESI) [M+H]⁺=1112.7.

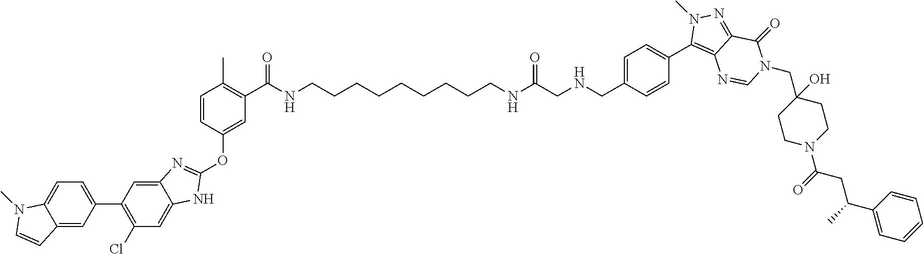 [SMILES: Cc1ccc(Oc2nc3cc(-c4ccc5c(ccn5C)c4)c(Cl)cc3[nH]2)cc1C(=O)NCCCCCCCCCNC(=O)CNCc1ccc(-c2c3ncn(CC4(O)CCN(C(=O)C[C@@H](C)c5ccccc5)CC4)c(=O)c3nn2C)cc1]

### Example 8: (R)-5-((6-chloro-5-(1-methyl-1H-indol-5-yl)-1H-benzo[d]imidazol-2-yl)oxy)-N-(9-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)nonyl)-2-methylbenzamide (XH181-8)

Example 8 was synthesized following the same procedure for preparing example 1 from intermediate 3 and linker 21. White solid, 1.9 mg, yield 9%, ¹H NMR (400 MHz, DMSO-d₆) δ 9.29 (s, 2H), 8.35-8.32 (m, 2H), 8.00-7.97 (m, 1H), 7.79 (d, J=8.0 Hz, 2H), 7.72-7.61 (m, 3H), 7.53-7.45 (m, 2H), 7.38-7.23 (m, 8H), 7.17-7.15 (m, 2H), 4.86 (s, 1H), 4.30-4.25 (m, 2H), 4.12 (s, 3H), 4.04-3.82 (m, 6H), 3.67-3.64 (m, 2H), 3.23-3.10 (m, 8H), 2.91-2.85 (m, 1H), 2.67-2.56 (m, 2H), 2.33 (s, 3H), 1.48-1.15 (m, 21H). MS (ESI) [M+H]⁺=1126.5.

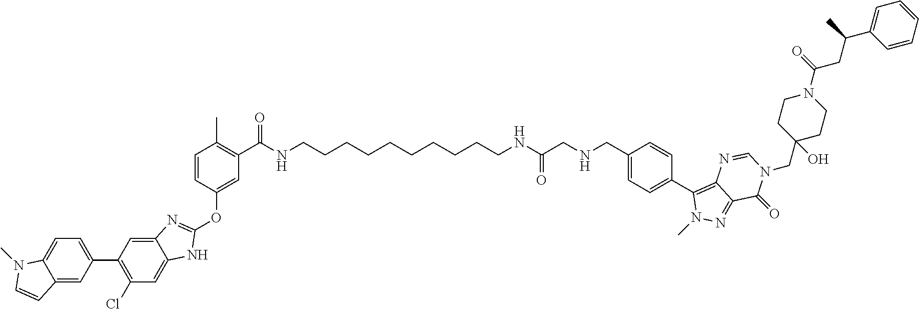 [SMILES: Cc1ccc(Oc2nc3cc(-c4ccc5c(ccn5C)c4)c(Cl)cc3[nH]2)cc1C(=O)NCCCCCCCCCCNC(=O)CNCc1ccc(-c2c3ncn(CC4(O)CCN(C(=O)C[C@@H](C)c5ccccc5)CC4)c(=O)c3nn2C)cc1]

### Example 9: (R)-5-((6-chloro-5-(1-methyl-1H-indol-5-yl)-1H-benzo[d]imidazol-2-yl)oxy)-N-(10-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)decyl)-2-methylbenzamide (XH181-9)

Example 9 was synthesized following the same procedure for preparing example 1 from intermediate 3 and linker 22. White solid, 2.3 mg, yield 11%, ¹H NMR (400 MHz, DMSO-d₆) δ 9.30 (s, 2H), 8.36-8.32 (m, 2H), 8.01-7.98 (m, 1H), 7.79 (d, J=8.0 Hz, 2H), 7.67 (d, J=8.0 Hz, 2H), 7.54-7.45 (m, 2H), 7.37-7.24 (m, 8H), 7.19-7.15 (m, 2H), 6.45 (d, J=3.7 Hz, 1H), 4.85 (s, 1H), 4.30-4.25 (m, 2H), 4.12 (s, 3H), 4.04-3.91 (m, 4H), 3.82 (s, 3H), 3.68 (s, 4H), 3.21-3.10 (m, 7H), 2.91-2.85 (m, 1H), 2.67-2.57 (m, 2H), 2.33 (s, 3H), 1.50-1.19 (m, 21H). MS (ESI) [M+H]⁺=1140.8.

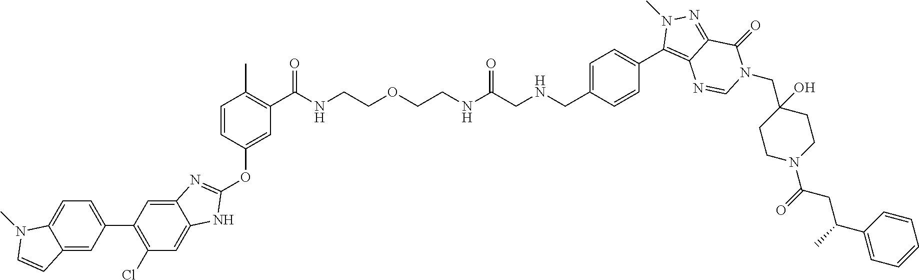 [SMILES: Cc1ccc(Oc2nc3cc(-c4ccc5c(ccn5C)c4)c(Cl)cc3[nH]2)cc1C(=O)NCCOCCNC(=O)CNCc1ccc(-c2c3ncn(CC4(O)CCN(C(=O)C[C@@H](C)c5ccccc5)CC4)c(=O)c3nn2C)cc1]

### Example 10: (R)-5-((6-chloro-5-(1-methyl-1H-indol-5-yl)-1H-benzo[d]imidazol-2-yl)oxy)-N-(2-(2-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)ethoxy)ethyl)-2-methylbenzamide (XH181-15)

Example 10 was synthesized following the same procedure for preparing example 1 from intermediate 3 and linker 23. White solid, 3.5 mg, yield 18%, ¹H NMR (400 MHz, Methanol-d₄) δ 7.98-7.82 (m, 1H), 7.68-7.49 (m, 6H), 7.44-7.12 (m, 14H), 4.22-4.13 (m, 2H), 4.06-4.00 (m, 2H), 3.97-3.94 (m, 3H), 3.87-3.80 (m, 2H), 3.73-3.67 (m, 2H), 3.63-3.53 (m, 8H), 3.43-3.38 (m, 2H), 3.23-3.09 (m, 2H), 3.05-2.76 (m, 2H), 2.73-2.60 (m, 2H), 2.53-2.42 (m, 4H), 1.59-1.45 (m, 1H), 1.34-1.24 (m, 6H). MS (ESI) [M+H]⁺=1072.7.

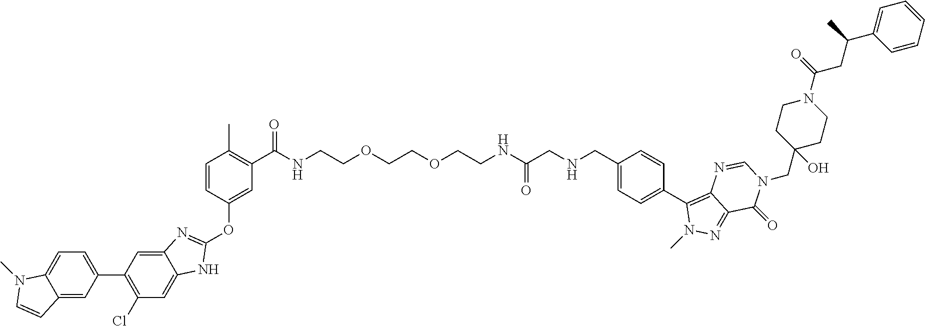 [SMILES: Cc1ccc(Oc2nc3cc(-c4ccc5c(ccn5C)c4)c(Cl)cc3[nH]2)cc1C(=O)NCCOCCOCCNC(=O)CNCc1ccc(-c2c3ncn(CC4(O)CCN(C(=O)C[C@@H](C)c5ccccc5)CC4)c(=O)c3nn2C)cc1]

### Example 11: (R)-5-((6-chloro-5-(1-methyl-1H-indol-5-yl)-1H-benzo[d]imidazol-2-yl)oxy)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11-dioxa-2,5-diazatridecan-13-yl)-2-methylbenzamide (Xh181-16)

Example 11 was synthesized following the same procedure for preparing example 1 from intermediate 3 and linker 24. White solid, 2.3 mg, yield 12%, 1H NMR (400 MHz, Methanol-d₄) δ 7.98-7.83 (m, 1H), 7.70-7.49 (m, 5H), 7.47-7.13 (m, 14H), 4.25-4.17 (m, 2H), 4.13-4.02 (m, 2H), 3.99-3.91 (m, 3H), 3.84-3.81 (m, 2H), 3.75-3.69 (m, 2H), 3.66-3.46 (m, 12H), 3.39-3.36 (m, 2H), 3.23-3.11 (m, 3H), 3.09-2.96 (m, 1H), 2.89-2.76 (m, 1H), 2.73-2.40 (m, 5H), 1.59-1.44 (m, 1H), 1.40-1.24 (m, 6H). MS (ESI) [M+H]⁺=1116.7.

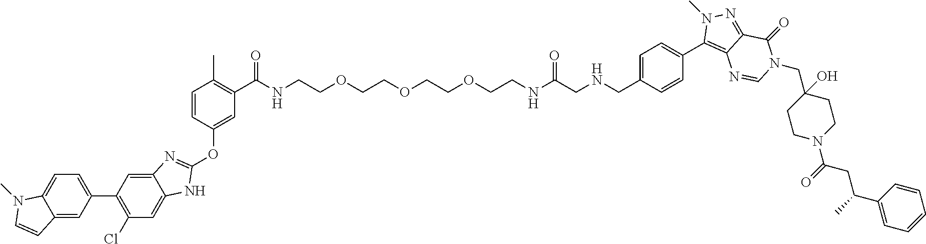 [SMILES: Cc1ccc(Oc2nc3cc(-c4ccc5c(ccn5C)c4)c(Cl)cc3[nH]2)cc1C(=O)NCCOCCOCCOCCNC(=O)CNCc1ccc(-c2c3ncn(CC4(O)CCN(C(=O)C[C@@H](C)c5ccccc5)CC4)c(=O)c3nn2C)cc1]

### Example 12: (R)-5-((6-chloro-5-(1-methyl-1H-indol-5-yl)-1H-benzo[d]imidazol-2-yl)oxy)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11,14-trioxa-2,5-diazahexadecan-16-yl)-2-methylbenzamide (XH181-17)

Example 12 was synthesized following the same procedure for preparing example 1 from intermediate 3 and linker 25. White solid, 4.0 mg, yield 19%, ¹H NMR (400 MHz, Methanol-d₄) δ 7.98-7.90 (m, 1H), 7.72-7.48 (m, 6H), 7.43-7.11 (m, 13H), 4.28-4.22 (m, 2H), 4.08-3.92 (m, 5H), 3.82 (s, 2H), 3.77-3.72 (m, 2H), 3.65-3.37 (m, 16H), 3.39-3.34 (m, 2H), 3.26-3.06 (m, 3H), 3.00-2.84 (m, 1H), 2.80-2.58 (m, 2H), 2.56-2.41 (m, 4H), 1.63-1.44 (m, 1H), 1.40-1.25 (m, 6H). MS (ESI) [M+H]⁺=1160.7.

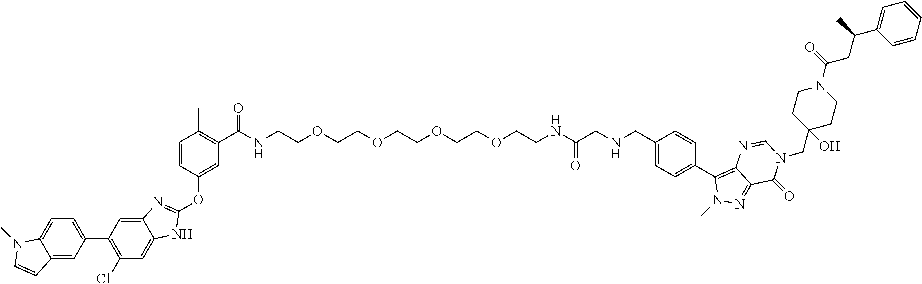 [SMILES: Cc1ccc(Oc2nc3cc(-c4ccc5c(ccn5C)c4)c(Cl)cc3[nH]2)cc1C(=O)NCCOCCOCCOCCOCCNC(=O)CNCc1ccc(-c2c3ncn(CC4(O)CCN(C(=O)C[C@@H](C)c5ccccc5)CC4)c(=O)c3nn2C)cc1]

### Example 13: (R)-5-((6-chloro-5-(1-methyl-1H-indol-5-yl)-1H-benzo[d]imidazol-2-yl)oxy)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11,14,17-tetraoxa-2,5-diazanonadecan-19-yl)-2-methylbenzamide (XH181-18)

Example 13 was synthesized following the same procedure for preparing example 1 from intermediate 3 and linker 26. White solid, 3.2 mg, yield 15%, ¹H NMR (400 MHz, Methanol-d₄) δ 7.98-7.86 (m, 1H), 7.73-7.57 (m, 4H), 7.52-7.12 (m, 15H), 4.33-4.24 (m, 2H), 4.16-4.02 (m, 5H), 3.98-3.88 (m, 2H), 3.85-3.82 (m, 2H), 3.79-3.72 (m, 2H), 3.64-3.46 (m, 20H), 3.41-3.34 (m, 2H), 3.24-3.07 (m, 2H), 3.00-2.84 (m, 1H), 2.79-2.67 (m, 1H), 2.65-2.41 (d, J=6.6 Hz, 4H), 1.59-1.47 (m, 1H), 1.41-1.22 (m, 6H). MS (ESI) [M+H]⁺=1204.8.

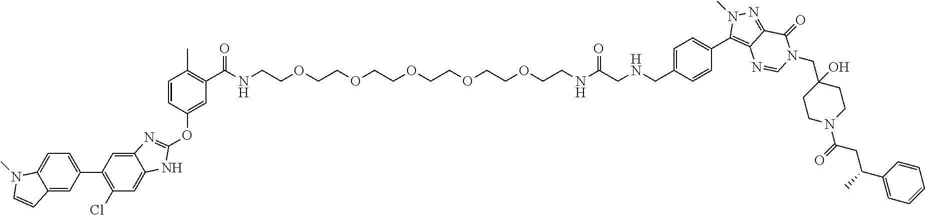 [SMILES: Cc1ccc(Oc2nc3cc(-c4ccc5c(ccn5C)c4)c(Cl)cc3[nH]2)cc1C(=O)NCCOCCOCCOCCOCCOCCNC(=O)CNCc1ccc(-c2c3ncn(CC4(O)CCN(C(=O)C[C@@H](C)c5ccccc5)CC4)c(=O)c3nn2C)cc1]

### Example 14: (R)-5-((6-chloro-5-(1-methyl-1H-indol-5-yl)-1H-benzo[d]imidazol-2-yl)oxy)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11,14,17,20-pentaoxa-2,5-diazadocosan-22-yl)-2-methylbenzamide (Xh181-19)

Example 14 was synthesized following the same procedure for preparing example 1 from intermediate 3 and linker 27. White solid, 3.2 mg, yield 14%, ¹H NMR (400 MHz, Methanol-d₄) δ 7.92-7.87 (m, 1H), 7.70-7.57 (m, 5H), 7.53-7.50 (m, 2H), 7.44-7.12 (m, 13H), 4.25-4.24 (m, 2H), 4.16-4.03 (m, 4H), 3.96 (s, 2H), 3.88-3.83 (m, 2H), 3.76-3.74 (m, 2H), 3.61-3.43 (m, 24H), 3.38-3.33 (m, 2H), 3.25-3.08 (m, 3H), 3.00-2.80 (m, 1H), 2.74-2.62 (m, 1H), 2.54-2.41 (m, 4H), 1.57-1.47 (m, 1H), 1.39-1.26 (m, 6H). MS (ESI) [M+H]⁺=1248.8.

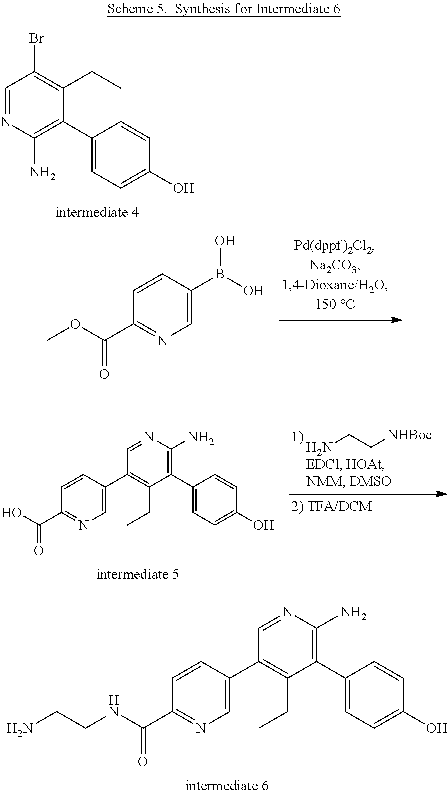 [SMILES: CCc1c(Br)cnc(N)c1-c1ccc(O)cc1 ; COC(=O)c1ccc(B(O)O)cn1 ; CCc1c(-c2ccc(C(=O)O)nc2)cnc(N)c1-c1ccc(O)cc1 ; CC(C)(C)OC(=O)NCCN ; CCc1c(-c2ccc(C(=O)NCCN)nc2)cnc(N)c1-c1ccc(O)cc1]

### Intermediate 6: 6′-amino-N-(2-aminoethyl)-4′-ethyl-5′-(4-hydroxyphenyl)-[3,3′-bipyridine]-6-carboxamide

To a solution of intermediate 4 (prepared following previous reported procedure) (Di Lello et al., 2017) (292 mg, 1 mmol, 1.0 equiv) in 1,4-Dioxane (3 mL), (6-(methoxycarbonyl)pyridin-3-yl) boronic acid (526 mg, 2 mmol, 2.0 equiv), Pd(dppf)Cl₂ (72.5 mg, 0.1 mmol, 10% mmol), saturated Na₂CO₃ aq. (3 mL) were added. The mixture was degassed with N₂ then heated under microwave irradiation for 4 h at 150° C. After cooling down to room temperature, the mixture was filtrated through a pad of celite, the filtrate was collected and evaporated in vacuo. The resulted residue was purified by reverse phase C18 column to afford intermediate 5 as white solid (67 mg, 15% yield). ¹H NMR (400 MHz, Methanol-d₄) δ 8.76 (d, J=3.3 Hz, 1H), 8.31 (dt, J=8.0, 2.3 Hz, 1H), 8.17-8.11 (m, 1H), 7.86 (t, J=2.6 Hz, 1H), 7.22 (dt, J=8.7, 2.4 Hz, 2H), 7.04 (dt, J=8.6, 2.3 Hz, 2H), 2.48 (h, J=6.4 Hz, 2H), 0.87-0.62 (m, 3H). MS (ESI) [M+H]⁺=336.3.

To a solution of intermediate 5 (300 mg, 0.89 mmol, 1.0 equiv) in DMSO (8 mL) were added tert-butyl (2-aminoethyl)carbamate (171 mg, 1.068 mmol, 1.2 equiv), EDCI (1-ethyl-3-(3-dimethylaminopropyl)carbodiimide) (256 mg, 1.335 mmol, 1.5 equiv), HOAt (1-hydroxy-7-azabenzo-triazole) (182 mg, 1.335 mmol, 1.5 equiv), and NMM (N-Methylmorpholine) (230 mg, 2.67 mmol, 3.0 equiv). After being stirred overnight at room temperature, the resulting mixture was purified by reverse phase C18 column to afford the crude product. The obtained solid was dissolved in DCM (2 mL) followed by TFA (1 mL). After being stirred for 1 h at room temperature, the reaction mixture was concentrated and purified by reverse phase C18 column to afford titled compound as white solid (162 mg, 48% yield for three steps). ¹H NMR (400 MHz, Methanol-d₄) δ 8.58 (d, J=2.1 Hz, 1H), 8.11 (d, J=8.0 Hz, 1H), 7.95 (dd, J=8.0, 2.2 Hz, 1H), 7.71 (s, 1H), 7.16-7.03 (m, 2H), 7.00-6.84 (m, 2H), 3.60 (dt, J=6.8, 3.2 Hz, 2H), 2.57 (q, J=6.7, 5.9 Hz, 2H), 2.34 (q, J=7.5 Hz, 2H), 0.62 (t, J=7.5 Hz, 3H). MS (ESI) [M+H]⁺=407.2.

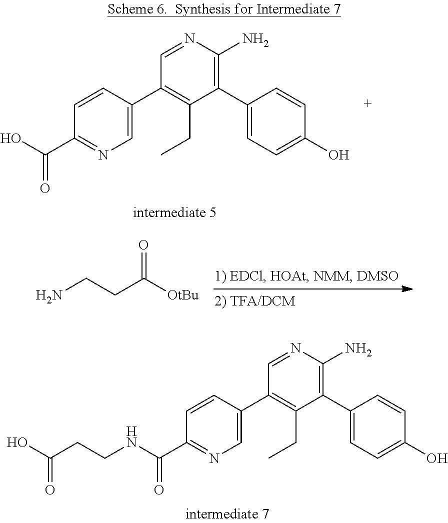 [SMILES: CCc1c(-c2ccc(C(=O)O)nc2)cnc(N)c1-c1ccc(O)cc1 ; CC(C)(C)OC(=O)CCN ; CCc1c(-c2ccc(C(=O)NCCC(=O)O)nc2)cnc(N)c1-c1ccc(O)cc1]

### Intermediate 7: 3-(6′-amino-4′-ethyl-5′-(4-hydroxyphenyl)-[3,3′-bipyridine]-6-carboxy-amido)propanoic acid

To a solution of intermediate 5 (300 mg, 0.89 mmol, 1.0 equiv) in DMSO (8 mL) were added tert-butyl 3-aminopropanoate (155 mg, 1.068 mmol, 1.2 equiv), EDCI (1-ethyl-3-(3-dimethylaminopropyl)carbodiimide) (256 mg, 1.335 mmol, 1.5 equiv), HOAt (1-hydroxy-7-azabenzo-triazole) (182 mg, 1.335 mmol, 1.5 equiv), and NMM (N-Methylmorpholine) (230 mg, 2.67 mmol, 3.0 equiv). After being stirred overnight at room temperature, the resulting mixture was purified by reverse phase C18 column to afford a white solid. The obtained solid was dissolved in DCM/TFA (2:1, 3 mL) TFA. After being stirred for 1 h at room temperature, the reaction mixture was concentrated and the residue was purified by reverse phase C18 column to afford titled compound as white solid (209 mg, 51% yield for two steps). ¹H NMR (400 MHz, Methanol-d₄) δ 8.62 (d, J=2.2 Hz, 1H), 8.14 (d, J=8.1 Hz, 1H), 7.98 (dd, J=8.1, 2.2 Hz, 1H), 7.71 (s, 1H), 7.16-7.01 (m, 2H), 6.99-6.85 (m, 2H), 3.64 (t, J=5.8 Hz, 2H), 3.10 (t, J=5.9 Hz, 2H), 2.34 (q, J=7.5 Hz, 2H), 0.61 (t, J=7.5 Hz, 3H). MS (ESI) [M+H]⁺=407.2.

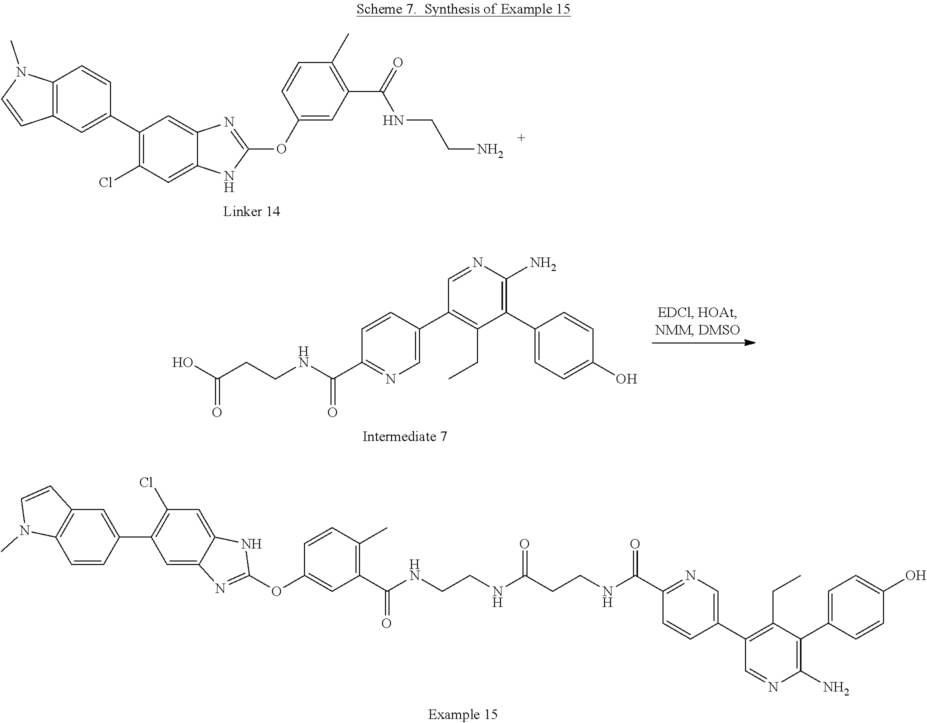 [SMILES: Cc1ccc(Oc2nc3cc(-c4ccc5c(ccn5C)c4)c(Cl)cc3[nH]2)cc1C(=O)NCCN ; CCc1c(-c2ccc(C(=O)NCCC(=O)O)nc2)cnc(N)c1-c1ccc(O)cc1 ; CCc1c(-c2ccc(C(=O)NCCC(=O)NCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 15: 6′-amino-N-(3-((2-(5-((6-chloro-5-(1-methyl-1H-indol-5-yl)-1H-benzo[d]imidazol-2-yl)oxy)-2-methylbenzamido)ethyl)amino)-3-oxopropyl)-4′-ethyl-5′-(4-hydroxyphenyl)-[3,3′-bipyridine]-6-carboxamide (KL186-1)

To a solution of intermediate 7 (4 mg, 0.01 mmol) in DMSO (1 mL) were added linker 14 (4.7 mg, 0.01 mmol, 1.0 equiv), EDCI (1-ethyl-3-(3-dimethylaminopropyl)carbodiimide) (3.0 mg, 0.015 mmol, 1.5 equiv), HOAt (1-hydroxy-7-azabenzo-triazole) (2.0 mg, 0.015 mmol, 1.5 equiv), and NMM (N-Methylmorpholine) (3.0 mg, 0.03 mmol, 3.0 equiv). After being stirred overnight at room temperature, the resulting mixture was purified by preparative HPLC to afford titled compound as pink solid (4.8 mg, 55%). ¹H NMR (400 MHz, Methanol-d₄) δ 8.50 (s, 1H), 8.05 (d, J=7.4 Hz, 1H), 7.87 (t, J=5.1 Hz, 1H), 7.61 (d, J=2.7 Hz, 1H), 7.43 (d, J=2.8 Hz, 1H), 7.34 (d, J=12.3 Hz, 2H), 7.29-7.22 (m, 4H), 7.10 (d, J=7.5 Hz, 2H), 7.02 (d, J=8.6 Hz, 2H), 6.88 (d, J=9.3 Hz, 2H), 6.34 (d, J=4.1 Hz, 1H), 3.74 (d, J=2.9 Hz, 3H), 3.64-3.57 (m, 2H), 2.43 (s, 2H), 2.33 (d, J=3.0 Hz, 3H), 1.67 (s, 4H), 0.56 (s, 3H). MS (ESI) [M+H]⁺=862.3.

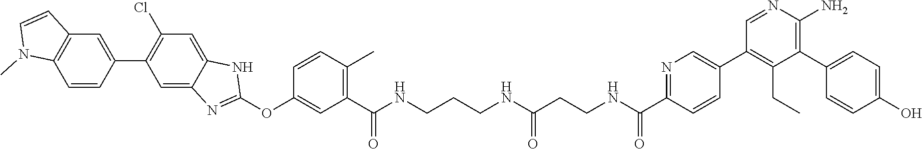 [SMILES: CCc1c(-c2ccc(C(=O)NCCC(=O)NCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 16: 6′-amino-N-(3-((3-(5-((6-chloro-5-(1-methyl-1H-indol-5-yl)-1H-benzo[d]imidazol-2-yl)oxy)-2-methylbenzamido)propyl)amino)-3-oxopropyl)-4′-ethyl-5′-(4-hydroxyphenyl)-[3,3′-bipyridine]-6-carboxamide (KL186-2)

Example 16 was synthesized following similar procedure for preparing example 15 from linker 15 and intermediate 7. Pink solid, 2.9 mg, 34% yield. ¹H NMR (400 MHz, Methanol-d₄) δ 8.50 (s, 1H), 8.05 (d, J=7.4 Hz, 1H), 7.87 (t, J=5.1 Hz, 1H), 7.61 (d, J=2.7 Hz, 1H), 7.43 (d, J=2.8 Hz, 1H), 7.34 (d, J=12.3 Hz, 2H), 7.29-7.22 (m, 4H), 7.10 (d, J=7.5 Hz, 2H), 7.02 (d, J=8.6 Hz, 2H), 6.88 (d, J=9.3 Hz, 2H), 6.34 (d, J=4.1 Hz, 1H), 3.74 (d, J=2.9 Hz, 3H), 3.64-3.57 (m, 2H), 2.43 (s, 2H), 2.33 (d, J=3.0 Hz, 3H), 2.28 (s, 2H), 1.67 (s, 4H), 0.56 (s, 3H). MS (ESI) m/z 876.5 [M+H]⁺.

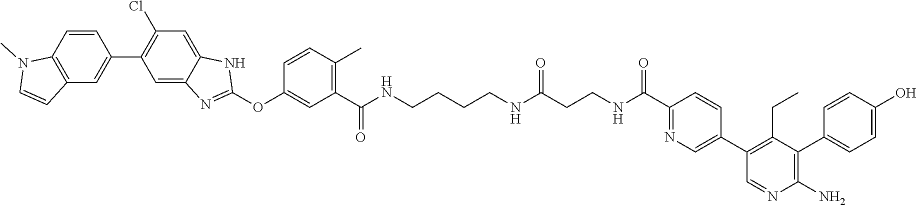 [SMILES: CCc1c(-c2ccc(C(=O)NCCC(=O)NCCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 17: 6′-amino-N-(3-((4-(5-((6-chloro-5-(1-methyl-1H-indol-5-yl)-1H-benzo[d]imidazol-2-yl)oxy)-2-methylbenzamido)butyl)amino)-3-oxopropyl)-4′-ethyl-5′-(4-hydroxyphenyl)-[3,3′-bipyridine]-6-carboxamide (KL186-3)

Example 17 was synthesized following similar procedure for preparing example 15 from linker 16 and intermediate 7. Pink solid, 2.3 mg, 26% yield. ¹H NMR (400 MHz, Methanol-d₄) δ 8.52 (s, 1H), 8.09-8.02 (m, 1H), 7.86 (d, J=8.0 Hz, 1H), 7.64 (d, J=2.8 Hz, 1H), 7.43 (d, J=3.1 Hz, 1H), 7.36 (d, J=2.9 Hz, 1H), 7.29-7.22 (m, 5H), 7.10 (d, J=8.9 Hz, 2H), 7.06-7.02 (m, 2H), 6.89 (d, J=8.6 Hz, 2H), 6.35 (d, J=2.9 Hz, 1H), 3.74 (d, J=2.8 Hz, 3H), 3.57 (d, J=8.0 Hz, 2H), 3.13 (s, 2H), 2.41 (s, 2H), 2.32 (d, J=2.9 Hz, 3H), 1.62 (s, 2H), 1.49 (s, 6H), 0.56 (d, J=9.0 Hz, 3H). MS (ESI) m/z 890.5 [M+H]⁺.

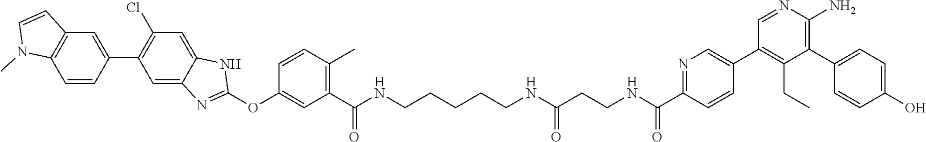 [SMILES: CCc1c(-c2ccc(C(=O)NCCC(=O)NCCCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 18: 6′-amino-N-(3-((5-(5-((6-chloro-5-(1-methyl-1H-indol-5-yl)-1H-benzo[d]imidazol-2-yl)oxy)-2-methylbenzamido)pentyl)amino)-3-oxopropyl)-4′-ethyl-5′-(4-hydroxyphenyl)-[3,3′-bipyridine]-6-carboxamide (KL186-4)

Example 18 was synthesized following similar procedure for preparing example 15 from linker 17 and intermediate 7. Pink solid, 2.8 mg, 31% yield. ¹H NMR (400 MHz, Methanol-d₄) δ 8.54 (d, J=3.1 Hz, 1H), 8.07 (d, J=8.1 Hz, 1H), 7.92-7.86 (m, 1H), 7.65 (d, J=3.2 Hz, 1H), 7.43 (d, J=3.3 Hz, 1H), 7.37 (d, J=2.9 Hz, 1H), 7.25 (t, J=8.0 Hz, 5H), 7.10 (d, J=9.2 Hz, 2H), 7.03 (d, J=8.9 Hz, 2H), 6.92-6.87 (m, 2H), 6.34 (d, J=3.7 Hz, 1H), 3.74 (t, J=2.8 Hz, 3H), 3.58 (d, J=5.5 Hz, 2H), 3.12-3.07 (m, 2H), 2.40 (q, J=6.2 Hz, 2H), 2.34-2.28 (m, 6H), 1.50 (s, 2H), 1.43 (s, 3H), 1.30 (s, 2H), 0.56 (d, J=9.6 Hz, 3H). MS (ESI) m/z 904.5 [M+H]⁺.

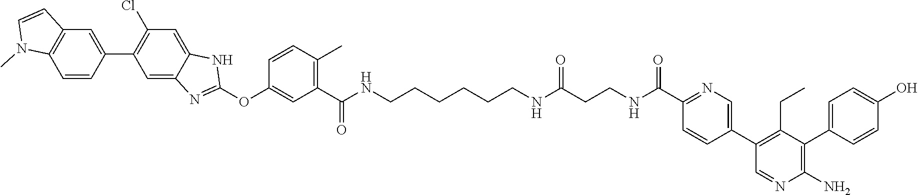 [SMILES: CCc1c(-c2ccc(C(=O)NCCC(=O)NCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 19: 6′-amino-N-(3-((6-(5-((6-chloro-5-(1-methyl-1H-indol-5-yl)-1H-benzo[d]imidazol-2-yl)oxy)-2-methylbenzamido)hexyl)amino)-3-oxopropyl)-4′-ethyl-5′-(4-hydroxyphenyl)-[3,3′-bipyridine]-6-carboxamide (KL186-5)

Example 19 was synthesized following similar procedure for preparing example 15 from linker 18 and intermediate 7. Pink solid, 3.0 mg, 33% yield. ¹H NMR (400 MHz, Methanol-d₄) δ 8.54 (d, J=3.2 Hz, 1H), 8.07 (d, J=8.9 Hz, 1H), 7.89 (d, J=8.2 Hz, 1H), 7.66 (d, J=2.9 Hz, 1H), 7.43 (d, J=3.5 Hz, 1H), 7.37 (d, J=2.9 Hz, 1H), 7.25 (dt, J=10.0, 4.8 Hz, 5H), 7.10 (d, J=9.8 Hz, 2H), 7.05 (d, J=8.7 Hz, 2H), 6.92-6.87 (m, 2H), 6.35 (d, J=2.7 Hz, 1H), 3.74 (t, J=2.7 Hz, 3H), 3.58 (d, J=5.6 Hz, 2H), 3.11-3.05 (m, 2H), 2.41 (q, J=6.0 Hz, 2H), 2.34-2.27 (m, 6H), 1.47 (s, 2H), 1.38 (s, 2H), 1.27 (s, 5H), 0.58 (q, J=9.1, 7.5 Hz, 3H). MS (ESI) m/z 918.5 [M+H]⁺.

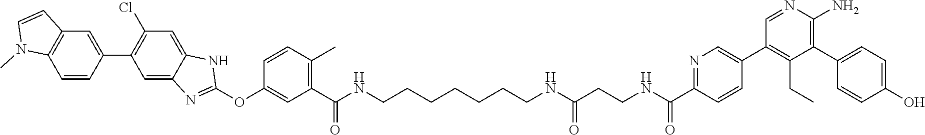 [SMILES: CCc1c(-c2ccc(C(=O)NCCC(=O)NCCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 20: 6′-amino-N-(3-((7-(5-((6-chloro-5-(1-methyl-1H-indol-5-yl)-1H-benzo[d]imidazol-2-yl)oxy)-2-methylbenzamido)heptyl)amino)-3-oxopropyl)-4′-ethyl-5′-(4-hydroxyphenyl)-[3,3′-bipyridine]-6-carboxamide (KL186-6)

Example 20 was synthesized following similar procedure for preparing example 15 from linker 19 and intermediate 7. Pink solid, 2.5 mg, 27% yield. ¹H NMR (400 MHz, Methanol-d₄) δ 8.54 (d, J=2.9 Hz, 1H), 8.10-8.04 (m, 1H), 7.89 (d, J=7.5 Hz, 1H), 7.66 (t, J=2.4 Hz, 1H), 7.44 (d, J=3.4 Hz, 1H), 7.36 (t, J=2.5 Hz, 1H), 7.29-7.21 (m, 5H), 7.11 (d, J=10.7 Hz, 2H), 7.05 (d, J=8.8 Hz, 2H), 6.91-6.87 (m, 2H), 6.35 (d, J=4.0 Hz, 1H), 3.74 (d, J=3.7 Hz, 3H), 3.62-3.55 (m, 2H), 3.05 (s, 2H), 2.41 (q, J=6.7, 6.0 Hz, 2H), 2.34-2.27 (m, 6H), 1.49 (s, 3H), 1.35 (s, 2H), 1.22 (d, J=14.8 Hz, 6H), 0.58 (q, J=6.6 Hz, 3H). MS (ESI) m/z 932.5 [M+H]⁺.

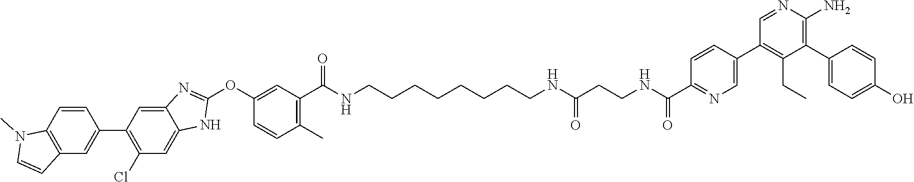 [SMILES: CCc1c(-c2ccc(C(=O)NCCC(=O)NCCCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 21: 6′-amino-N-(3-((8-(5-((6-chloro-5-(1-methyl-1H-indol-5-yl)-1H-benzo[d]imidazol-2-yl)oxy)-2-methylbenzamido)octyl)amino)-3-oxopropyl)-4′-ethyl-5′-(4-hydroxyphenyl)-[3,3′-bipyridine]-6-carboxamide (KL186-7)

Example 21 was synthesized following similar procedure for preparing example 15 from linker 20 and intermediate 7. Pink solid, 3.4 mg, 36% yield. ¹H NMR (400 MHz, Methanol-d₄) δ 8.66 (d, J=3.0 Hz, 1H), 8.24-8.18 (m, 1H), 8.03 (dd, J=8.5, 3.0 Hz, 1H), 7.79 (d, J=2.9 Hz, 1H), 7.57 (d, J=3.4 Hz, 1H), 7.50 (d, J=3.0 Hz, 1H), 7.41-7.35 (m, 5H), 7.23 (d, J=10.6 Hz, 2H), 7.20-7.16 (m, 2H), 7.02 (d, J=7.3 Hz, 2H), 6.48 (d, J=4.8 Hz, 1H), 3.87 (t, J=2.7 Hz, 3H), 3.78 (s, 1H), 3.71 (q, J=8.2, 6.8 Hz, 2H), 3.16 (d, J=11.1 Hz, 2H), 2.53 (p, J=5.2 Hz, 2H), 2.49-2.41 (m, 6H), 1.62 (s, 3H), 1.36 (d, J=32.1 Hz, 9H), 0.71 (q, J=8.4, 7.0 Hz, 3H). MS (ESI) m/z 946.5 [M+H]⁺.

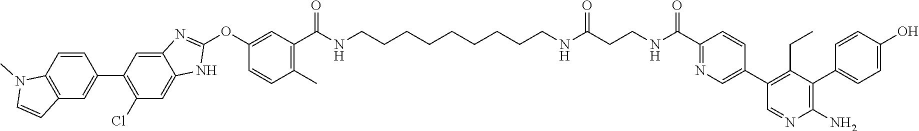 [SMILES: CCc1c(-c2ccc(C(=O)NCCC(=O)NCCCCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 22: 6′-amino-N-(3-((9-(5-((6-chloro-5-(1-methyl-1H-indol-5-yl)-1H-benzo[d]imidazol-2-yl)oxy)-2-methylbenzamido)nonyl)amino)-3-oxopropyl)-4′-ethyl-5′-(4-hydroxyphenyl)-[3,3′-bipyridine]-6-carboxamide (KL186-8)

Example 22 was synthesized following similar procedure for preparing example 15 from linker 21 and intermediate 7. Pink solid, 3.6 mg, 38% yield. ¹H NMR (400 MHz, Methanol-d₄) δ 8.53 (d, J=3.1 Hz, 1H), 8.13-8.04 (m, 1H), 7.95-7.86 (m, 1H), 7.66 (d, J=3.0 Hz, 1H), 7.44 (t, J=2.4 Hz, 1H), 7.36 (d, J=2.9 Hz, 1H), 7.33-7.18 (m, 5H), 7.15-7.03 (m, 4H), 6.94-6.86 (m, 2H), 6.35 (q, J=3.0 Hz, 1H), 3.74 (t, J=3.0 Hz, 3H), 3.62-3.56 (m, 2H), 3.29-3.23 (m, 2H), 3.04 (q, J=6.2, 5.8 Hz, 2H), 2.41 (p, J=5.1 Hz, 2H), 2.37-2.26 (m, 5H), 1.50 (d, J=10.6 Hz, 2H), 1.39-1.30 (m, 2H), 1.25 (s, 4H), 1.16 (s, 6H), 0.58 (d, J=9.6 Hz, 3H). MS (ESI) m/z 960.3 [M+H]⁺.

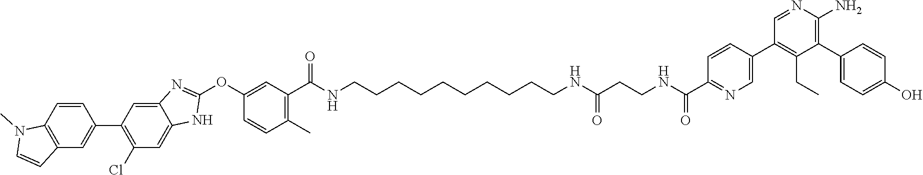 [SMILES: CCc1c(-c2ccc(C(=O)NCCC(=O)NCCCCCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 23: 6′-amino-N-(3-((10-(5-((6-chloro-5-(1-methyl-1H-indol-5-yl)-1H-benzo[d]imidazol-2-yl)oxy)-2-methylbenzamido)decyl)amino)-3-oxopropyl)-4′-ethyl-5′-(4-hydroxyphenyl)-[3,3′-bipyridine]-6-carboxamide (KL186-9)

Example 23 was synthesized following similar procedure for preparing example 15 from linker 22 and intermediate 7. Pink solid, 2.0 mg, 21% yield. ¹H NMR (400 MHz, Methanol-d₄) δ 8.53 (s, 1H), 8.07 (t, J=6.2 Hz, 1H), 7.95-7.86 (m, 1H), 7.67 (d, J=2.8 Hz, 1H), 7.44 (d, J=3.5 Hz, 1H), 7.37 (d, J=2.8 Hz, 1H), 7.31-7.21 (m, 5H), 7.11 (d, J=10.8 Hz, 2H), 7.05 (d, J=8.3 Hz, 2H), 6.93-6.88 (m, 2H), 6.35 (d, J=4.1 Hz, 1H), 3.74 (t, J=2.9 Hz, 3H), 3.59 (d, J=5.7 Hz, 2H), 3.26 (s, 2H), 3.04 (d, J=5.8 Hz, 2H), 2.41 (q, J=6.1 Hz, 2H), 2.37-2.25 (m, 6H), 1.49 (d, J=9.2 Hz, 3H), 1.31 (d, J=22.8 Hz, 6H), 1.17-1.12 (m, 6H), 0.58 (d, J=10.0 Hz, 3H). MS (ESI) m/z 974.5 [M+H]⁺.

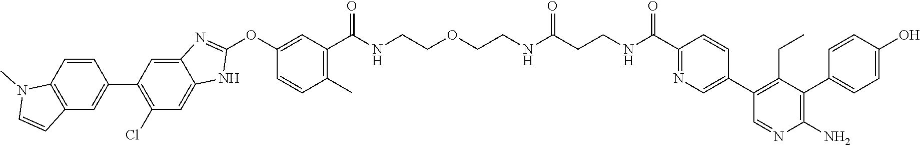 [SMILES: CCc1c(-c2ccc(C(=O)NCCC(=O)NCCOCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 24: 6′-amino-N-(3-((2-(2-(5-((6-chloro-5-(1-methyl-1H-indol-5-yl)-1H-benzo[d]imidazol-2-yl)oxy)-2-methylbenzamido)ethoxy)ethyl)amino)-3-oxopropyl)-4′-ethyl-5′-(4-hydroxyphenyl)-[3,3′-bipyridine]-6-carboxamide (KL186-10)

Example X24 was synthesized following similar procedure for preparing example 15 from linker 23 and intermediate 7. Pink solid, 3.2 mg, 36% yield. ¹H NMR (400 MHz, Methanol-d₄) δ 8.48 (d, J=3.2 Hz, 1H), 8.06-8.00 (m, 1H), 7.88-7.83 (m, 1H), 7.63 (d, J=3.0 Hz, 1H), 7.44-7.40 (m, 1H), 7.35 (d, J=3.1 Hz, 1H), 7.32-7.23 (m, 5H), 7.09 (d, J=4.5 Hz, 2H), 7.05-7.01 (m, 2H), 6.89 (d, J=8.3 Hz, 2H), 6.35 (s, 1H), 3.74 (t, J=2.7 Hz, 3H), 3.50 (q, J=5.5, 4.9 Hz, 4H), 3.42 (d, J=7.0 Hz, 4H), 3.27 (d, J=4.7 Hz, 2H), 2.36 (d, J=5.5 Hz, 2H), 2.33-2.30 (m, 4H), 1.20 (t, J=2.7 Hz, 1H), 0.56 (q, J=8.7, 7.3 Hz, 3H). MS (ESI) m/z 905.2 [M+H]⁺.

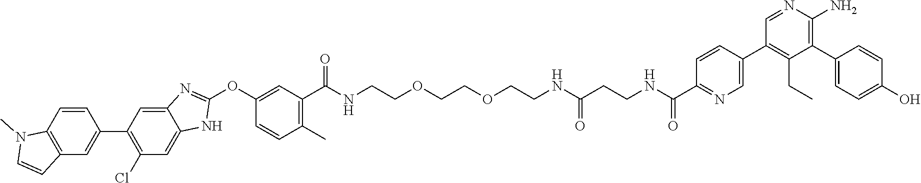 [SMILES: CCc1c(-c2ccc(C(=O)NCCC(=O)NCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 25: 6′-amino-N-(1-(5-((6-chloro-5-(1-methyl-1H-indol-5-yl)-1H-benzo[d]imidazol-2-yl)oxy)-2-methylphenyl)-1,12-dioxo-5,8-dioxa-2,11-diazatetradecan-14-yl)-4′-ethyl-5′-(4-hydroxyphenyl)-[3,3′-bipyridine]-6-carboxamide (KL186-11)

Example 25 was synthesized following similar procedure for preparing example 15 from linker 24 and intermediate 7. Pink solid, 3.1 mg, 33% yield. ¹H NMR (400 MHz, Methanol-d₄) δ 8.47 (s, 1H), 8.07-8.01 (m, 1H), 7.86 (dd, J=7.7, 3.4 Hz, 1H), 7.63 (d, J=3.3 Hz, 1H), 7.43 (d, J=3.3 Hz, 1H), 7.36 (t, J=2.3 Hz, 1H), 7.32-7.20 (m, 6H), 7.09 (t, J=5.2 Hz, 2H), 7.05-7.01 (m, 2H), 6.88 (dd, J=8.8, 3.1 Hz, 2H), 3.78-3.68 (m, 3H), 3.55 (t, J=5.0 Hz, 4H), 3.52-3.49 (m, 2H), 3.45 (s, 4H), 3.37 (d, J=5.0 Hz, 2H), 2.39 (q, J=5.9, 5.1 Hz, 2H), 2.35-2.25 (m, 6H), 1.19 (d, J=2.8 Hz, 1H), 0.56 (q, J=6.8, 6.3 Hz, 3H). MS (ESI) m/z 950.5 [M+H]⁺.

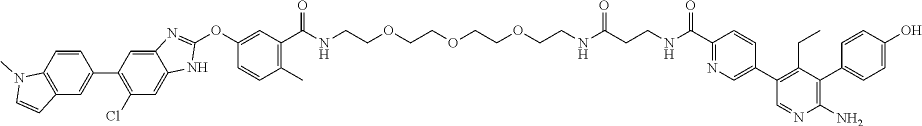 [SMILES: CCc1c(-c2ccc(C(=O)NCCC(=O)NCCOCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 26: 6′-amino-N-(1-(5-((6-chloro-5-(1-methyl-1H-indol-5-yl)-1H-benzo[d]imidazol-2-yl)oxy)-2-methylphenyl)-1,15-dioxo-5,8,11-trioxa-2,14-diazaheptadecan-17-yl)-4′-ethyl-5′-(4-hydroxyphenyl)-[3,3′-bipyridine]-6-carboxamide (KL186-12)

Example 26 was synthesized following similar procedure for preparing example 15 from linker 25 and intermediate 7. Pink solid, 3.7 mg, 38% yield. ¹H NMR (400 MHz, Methanol-d₄) δ 8.49 (d, J=3.3 Hz, 1H), 8.04 (q, J=3.2 Hz, 1H), 7.90-7.82 (m, 1H), 7.64 (d, J=2.8 Hz, 1H), 7.46-7.41 (m, 1H), 7.36 (d, J=2.9 Hz, 1H), 7.31-7.27 (m, 2H), 7.26-7.21 (m, 4H), 7.10 (t, J=5.7 Hz, 2H), 7.06-7.01 (m, 2H), 6.92-6.87 (m, 2H), 6.35 (d, J=4.5 Hz, 1H), 3.74 (t, J=2.7 Hz, 3H), 3.53 (dt, J=21.8, 7.5 Hz, 8H), 3.45-3.41 (m, 4H), 3.38-3.33 (m, 4H), 2.46-2.39 (m, 2H), 2.32 (q, J=8.9, 4.9 Hz, 6H), 1.20 (t, J=2.8 Hz, 1H), 0.57 (q, J=9.2, 7.8 Hz, 3H). MS (ESI) m/z 994.5 [M+H]⁺.

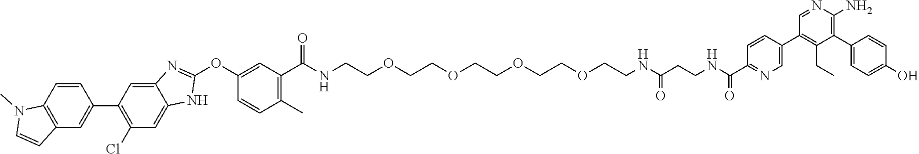 [SMILES: CCc1c(-c2ccc(C(=O)NCCC(=O)NCCOCCOCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 27: 6′-amino-N-(1-(5-((6-chloro-5-(1-methyl-1H-indol-5-yl)-1H-benzo[d]imidazol-2-yl)oxy)-2-methylphenyl)-1,18-dioxo-5,8,11,14-tetraoxa-2,17-diazaicosan-20-yl)-4′-ethyl-5′-(4-hydroxyphenyl)-[3,3′-bipyridine]-6-carboxamide (KL186-13)

Example 27 was synthesized following similar procedure for preparing example 15 from linker 26 and intermediate 7. Pink solid, 3.5 mg, 34% yield. ¹H NMR (400 MHz, Methanol-d₄) δ 8.60 (d, J=3.5 Hz, 1H), 8.22-8.11 (m, 1H), 8.01-7.90 (m, 1H), 7.75 (t, J=2.9 Hz, 1H), 7.59-7.50 (m, 1H), 7.48 (d, J=3.0 Hz, 1H), 7.44-7.31 (m, 5H), 7.23-7.17 (m, 2H), 7.17-7.10 (m, 2H), 7.00 (tt, J=7.6, 4.9 Hz, 2H), 6.45 (q, J=3.1 Hz, 1H), 3.89-3.78 (m, 3H), 3.65 (ddt, J=24.8, 14.7, 5.0 Hz, 9H), 3.56-3.46 (m, 12H), 2.53 (dt, J=8.7, 5.3 Hz, 2H), 2.44 (d, J=3.0 Hz, 3H), 2.37 (q, J=7.4, 6.4 Hz, 2H), 0.67 (dd, J=8.2, 4.7 Hz, 3H). MS (ESI) m/z 1038.3 [M+H]⁺.

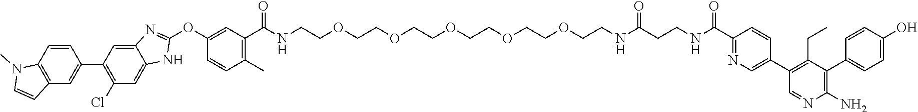 [SMILES: CCc1c(-c2ccc(C(=O)NCCC(=O)NCCOCCOCCOCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 28: 6′-amino-N-(1-(5-((6-chloro-5-(1-methyl-1H-indol-5-yl)-1H-benzo[d]imidazol-2-yl)oxy)-2-methylphenyl)-1,21-dioxo-5,8,11,14,17-pentaoxa-2,20-diazatricosan-23-yl)-4′-ethyl-5′-(4-hydroxyphenyl)-[3,3′-bipyridine]-6-carboxamide (KL186-14)

Example 28 was synthesized following similar procedure for preparing example 15 from linker 27 and intermediate 7. Pink solid, 2.0 mg, 19% yield. ¹H NMR (400 MHz, Methanol-d₄) δ 8.51 (d, J=3.1 Hz, 1H), 8.06 (d, J=8.6 Hz, 1H), 7.91-7.85 (m, 1H), 7.68-7.64 (m, 1H), 7.44 (t, J=2.4 Hz, 1H), 7.37 (t, J=2.5 Hz, 1H), 7.31-7.23 (m, 5H), 7.10 (d, J=9.2 Hz, 2H), 7.04 (d, J=10.1 Hz, 2H), 6.92-6.87 (m, 2H), 6.35 (s, 1H), 3.74 (t, J=2.7 Hz, 3H), 3.55-3.48 (m, 8H), 3.40 (d, J=13.9 Hz, 16H), 2.42 (d, J=8.3 Hz, 2H), 2.35-2.27 (m, 6H), 1.21-1.19 (m, 1H), 0.57 (d, J=9.6 Hz, 3H). MS (ESI) m/z 1083.2 [M+H]⁺.

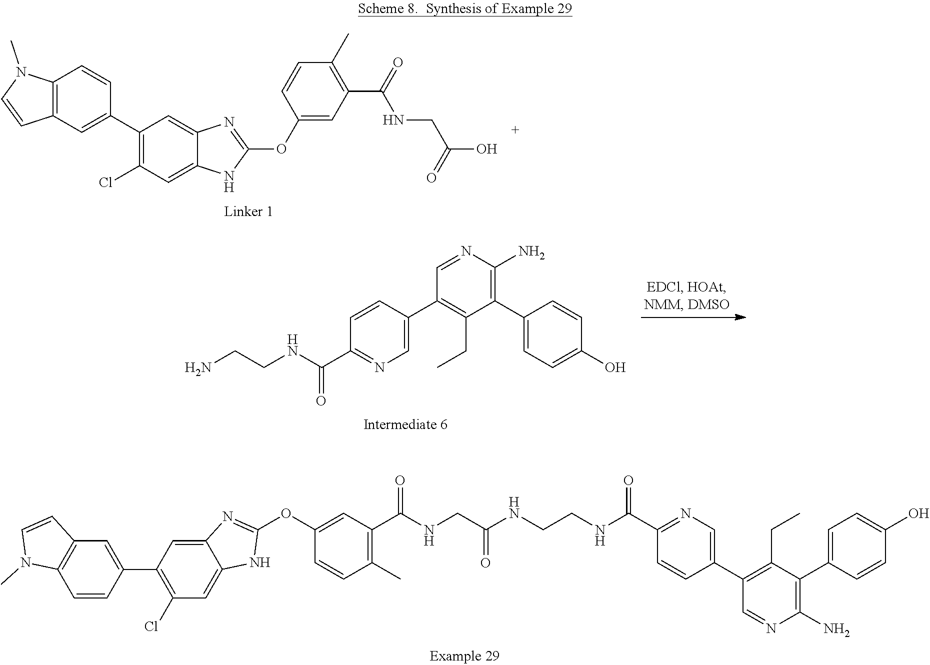 [SMILES: Cc1ccc(Oc2nc3cc(-c4ccc5c(ccn5C)c4)c(Cl)cc3[nH]2)cc1C(=O)NCC(=O)O ; CCc1c(-c2ccc(C(=O)NCCN)nc2)cnc(N)c1-c1ccc(O)cc1 ; CCc1c(-c2ccc(C(=O)NCCNC(=O)CNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 29: 6′-amino-N-(2-(2-(5-((6-chloro-5-(1-methyl-1H-indol-5-yl)-1H-benzo[d]imidazol-2-yl)oxy)-2-methylbenzamido)acetamido)ethyl)-4′-ethyl-5′-(4-hydroxyphenyl)-[3,3′-bipyridine]-6-carboxamide (KL186-48)

To a solution of intermediate 6 (3.8 mg, 0.01 mmol) in DMSO (1 mL) were added linker 1 (4.9 mg, 0.01 mmol, 1.0 equiv), EDCI (1-ethyl-3-(3-dimethylaminopropyl)carbodiimide) (3.0 mg, 0.015 mmol, 1.5 equiv), HOAt (1-hydroxy-7-azabenzo-triazole) (2.0 mg, 0.015 mmol, 1.5 equiv), and NMM (N-Methylmorpholine) (3.0 mg, 0.03 mmol, 3.0 equiv). After being stirred overnight at room temperature, the resulting mixture was purified by preparative HPLC to afford titled compound as pink solid (4.8 mg, 57%). ¹H NMR (400 MHz, Methanol-d₄) δ 8.50 (d, J=3.8 Hz, 1H), 7.89 (dt, J=6.2, 2.8 Hz, 1H), 7.70 (d, J=3.5 Hz, 1H), 7.62 (t, J=2.8 Hz, 1H), 7.58-7.51 (m, 1H), 7.43 (d, J=3.8 Hz, 1H), 7.37 (t, J=2.8 Hz, 1H), 7.33-7.22 (m, 4H), 7.14-7.04 (m, 2H), 6.97-6.89 (m, 2H), 6.89-6.82 (m, 2H), 6.36 (q, J=3.0 Hz, 1H), 3.99-3.86 (m, 2H), 3.81-3.69 (m, 3H), 3.51 (t, J=5.1 Hz, 2H), 3.39 (d, J=5.4 Hz, 2H), 2.36 (t, J=2.7 Hz, 3H), 2.26-2.19 (m, 2H), 0.55-0.50 (m, 3H). MS (ESI) m/z 848.3 [M+H]⁺.

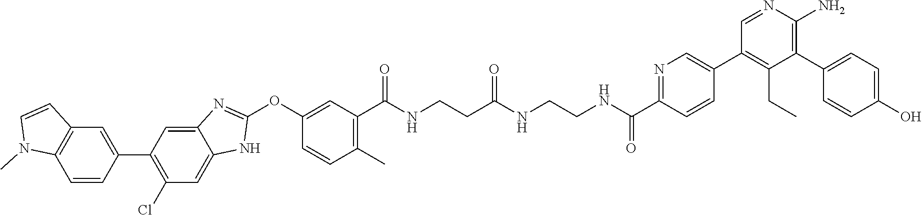 [SMILES: CCc1c(-c2ccc(C(=O)NCCNC(=O)CCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 30: 6′-amino-N-(2-(3-(5-((6-chloro-5-(1-methyl-1H-indol-5-yl)-1H-benzo[d]imidazol-2-yl)oxy)-2-methylbenzamido)propanamido)ethyl)-4′-ethyl-5′-(4-hydroxyphenyl)-[3,3′-bipyridine]-6-carboxamide (KL186-49)

Example 30 was synthesized following similar procedure for preparing example 29 from linker 2 and intermediate 6. Pink solid, 4.9 mg, 57% yield. ¹H NMR (400 MHz, Methanol-d₄) δ 8.58 (d, J=3.6 Hz, 1H), 8.12-8.02 (m, 1H), 7.93-7.85 (m, 1H), 7.69 (d, J=3.5 Hz, 1H), 7.53 (d, J=3.4 Hz, 1H), 7.44 (d, J=3.5 Hz, 2H), 7.42-7.28 (m, 4H), 7.25-7.17 (m, 2H), 7.15-7.09 (m, 2H), 7.05-6.95 (m, 2H), 6.46 (q, J=2.9 Hz, 1H), 3.85 (d, J=3.2 Hz, 3H), 3.67-3.64 (m, 2H), 3.50 (t, J=5.1 Hz, 2H), 3.42 (t, J=5.0 Hz, 2H), 2.56-2.51 (m, 2H), 2.44 (d, J=3.5 Hz, 3H), 2.40-2.27 (m, 2H), 0.65 (q, J=6.5 Hz, 3H). MS (ESI) m/z 862.3 [M+H]⁺.

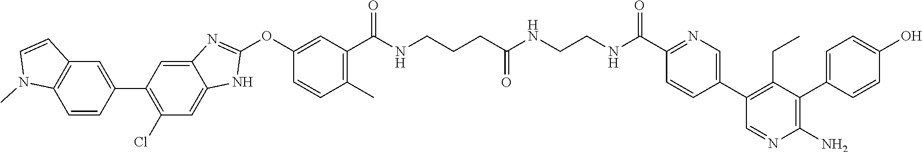 [SMILES: CCc1c(-c2ccc(C(=O)NCCNC(=O)CCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 31: 6′-amino-N-(2-(4-(5-((6-chloro-5-(1-methyl-1H-indol-5-yl)-1H-benzo[d]imidazol-2-yl)oxy)-2-methylbenzamido)butanamido)ethyl)-4′-ethyl-5′-(4-hydroxyphenyl)-[3,3′-bipyridine]-6-carboxamide (KL186-50)

Example 31 was synthesized following similar procedure for preparing example 29 from linker 3 and intermediate 6. Pink solid, 5.5 mg, 63% yield. ¹H NMR (400 MHz, Methanol-d₄) δ 8.62 (d, J=3.7 Hz, 1H), 8.18-8.09 (m, 1H), 7.99-7.89 (m, 1H), 7.72 (t, J=2.7 Hz, 1H), 7.54 (d, J=3.6 Hz, 1H), 7.51-7.43 (m, 2H), 7.43-7.33 (m, 4H), 7.21 (q, J=4.0, 3.0 Hz, 2H), 7.17-7.10 (m, 2H), 7.00 (dt, J=8.4, 2.3 Hz, 2H), 6.47 (q, J=2.9 Hz, 1H), 3.91-3.80 (m, 3H), 3.56 (t, J=5.2 Hz, 2H), 3.46 (t, J=5.2 Hz, 2H), 3.39 (dd, J=7.5, 4.3 Hz, 2H), 2.44 (t, J=2.6 Hz, 3H), 2.42-2.30 (m, 4H), 1.93-1.87 (m, 2H), 0.69-0.65 (m, 3H). MS (ESI) m/z 876.3 [M+H]⁻.

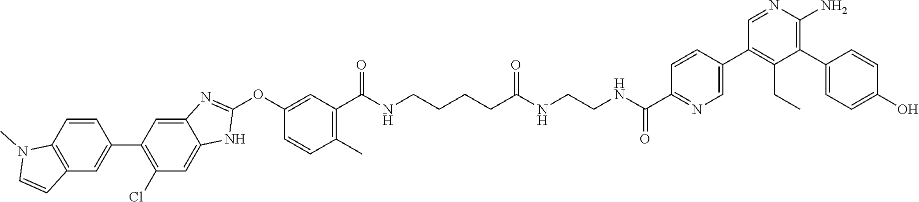 [SMILES: CCc1c(-c2ccc(C(=O)NCCNC(=O)CCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 32: 6′-amino-N-(2-(5-(5-((6-chloro-5-(1-methyl-1H-indol-5-yl)-1H-benzo[d]imidazol-2-yl)oxy)-2-methylbenzamido)pentanamido)ethyl)-4′-ethyl-5′-(4-hydroxyphenyl)-[3,3′-bipyridine]-6-carboxamide (KL186-51)

Example 32 was synthesized following similar procedure for preparing example 29 from linker 4 and intermediate 6. Pink solid, 5.2 mg, 58% yield. ¹H NMR (400 MHz, Methanol-d₄) δ 8.65 (d, J=3.5 Hz, 1H), 8.19-8.11 (m, 1H), 7.99-7.92 (m, 1H), 7.75 (t, J=2.7 Hz, 1H), 7.55 (d, J=3.6 Hz, 1H), 7.49 (t, J=2.8 Hz, 1H), 7.43-7.33 (m, 5H), 7.22 (t, J=5.7 Hz, 2H), 7.14 (t, J=5.3 Hz, 2H), 7.04-6.97 (m, 2H), 6.47 (q, J=3.0 Hz, 1H), 3.91-3.80 (m, 3H), 3.55 (t, J=5.2 Hz, 2H), 3.45 (t, J=5.3 Hz, 2H), 3.37 (s, 2H), 2.44 (t, J=2.8 Hz, 3H), 2.38 (q, J=7.3, 6.6 Hz, 2H), 2.29-2.24 (m, 2H), 1.78-1.58 (m, 4H), 0.71-0.66 (m, 3H). MS (ESI) m/z 890.3 [M+H]⁺.

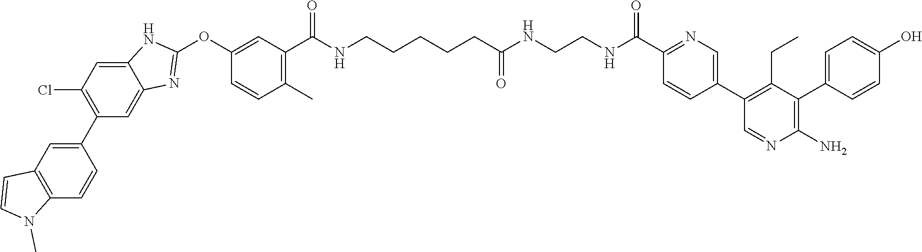 [SMILES: CCc1c(-c2ccc(C(=O)NCCNC(=O)CCCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 33: 6′-amino-N-(2-(6-(5-((6-chloro-5-(1-methyl-1H-indol-5-yl)-1H-benzo[d]imidazol-2-yl)oxy)-2-methylbenzamido)hexanamido)ethyl)-4′-ethyl-5′-(4-hydroxyphenyl)-[3,3′-bipyridine]-6-carboxamide (KL186-52)

Example 33 was synthesized following similar procedure for preparing example 29 from linker 5 and intermediate 6. Pink solid, 5.8 mg, 64% yield. ¹H NMR (400 MHz, Methanol-d₄) δ 8.66 (d, J=3.8 Hz, 1H), 8.23-8.10 (m, 1H), 7.98 (dd, J=7.2, 3.9 Hz, 1H), 7.76 (t, J=2.8 Hz, 1H), 7.55 (d, J=3.8 Hz, 1H), 7.48 (t, J=2.8 Hz, 1H), 7.43-7.32 (m, 5H), 7.26-7.18 (m, 2H), 7.18-7.11 (m, 2H), 7.01 (dt, J=7.5, 2.9 Hz, 2H), 6.47 (q, J=3.1 Hz, 1H), 3.95-3.78 (m, 3H), 3.55 (t, J=5.4 Hz, 2H), 3.44 (q, J=5.6 Hz, 2H), 2.46-2.41 (m, 3H), 2.39 (q, J=7.4, 6.0 Hz, 2H), 2.25-2.20 (m, 2H), 1.71-1.55 (m, 4H), 1.45-1.38 (m, 2H), 0.71-0.66 (m, 3H). MS (ESI) m/z 904.4 [M+H]⁺.

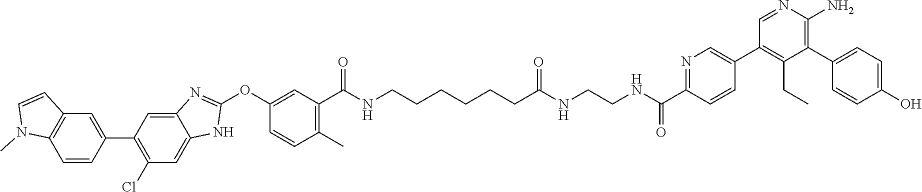 [SMILES: CCc1c(-c2ccc(C(=O)NCCNC(=O)CCCCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 34: 6′-amino-N-(2-(7-(5-((6-chloro-5-(1-methyl-1H-indol-5-yl)-1H-benzo[d]imidazol-2-yl)oxy)-2-methylbenzamido)heptanamido)ethyl)-4′-ethyl-5′-(4-hydroxyphenyl)-[3,3′-bipyridine]-6-carboxamide (KL186-53)

Example 34 was synthesized following similar procedure for preparing example 29 from linker 6 and intermediate 6. Pink solid, 4.8 mg, 52% yield. ¹H NMR (400 MHz, Methanol-d₄) δ 8.67 (d, J=3.6 Hz, 1H), 8.18 (dt, J=6.8, 2.9 Hz, 1H), 8.04-7.96 (m, 1H), 7.77 (t, J=2.7 Hz, 1H), 7.60-7.53 (m, 1H), 7.49 (t, J=2.6 Hz, 1H), 7.44-7.32 (m, 5H), 7.23 (dd, J=10.5, 3.0 Hz, 2H), 7.19-7.13 (m, 2H), 7.05-6.96 (m, 2H), 6.47 (q, J=3.1 Hz, 1H), 3.91-3.80 (m, 3H), 3.56 (t, J=5.4 Hz, 2H), 3.46 (t, J=5.4 Hz, 2H), 3.35 (s, 2H), 2.44-2.38 (m, 5H), 2.19 (q, J=5.7, 4.7 Hz, 2H), 1.63-1.56 (m, 4H), 1.37 (s, 4H), 0.73-0.68 (m, 3H). MS (ESI) m/z 918.4 [M+H]⁻.

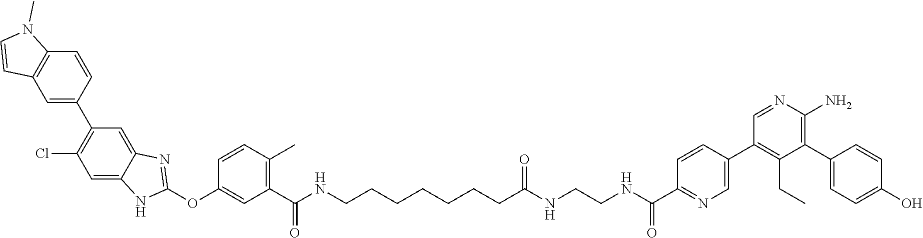 [SMILES: CCc1c(-c2ccc(C(=O)NCCNC(=O)CCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 35: 6′-amino-N-(2-(8-(5-((6-chloro-5-(1-methyl-1H-indol-5-yl)-1H-benzo[d]imidazol-2-yl)oxy)-2-methylbenzamido)octanamido)ethyl)-4′-ethyl-5′-(4-hydroxyphenyl)-[3,3′-bipyridine]-6-carboxamide (KL186-54)

Example 35 was synthesized following similar procedure for preparing example 29 from linker 7 and intermediate 6. Pink solid, 6.1 mg, 65% yield. ¹H NMR (400 MHz, Methanol-d₄) δ 8.67 (d, J=3.6 Hz, 1H), 8.24-8.14 (m, 1H), 8.01 (dd, J=7.9, 3.8 Hz, 1H), 7.78 (t, J=2.8 Hz, 1H), 7.56 (d, J=3.7 Hz, 1H), 7.49 (t, J=2.7 Hz, 1H), 7.46-7.27 (m, 5H), 7.26-7.19 (m, 2H), 7.19-7.13 (m, 2H), 7.04-6.97 (m, 2H), 6.47 (q, J=3.0 Hz, 1H), 3.90-3.80 (m, 3H), 3.56 (t, J=5.3 Hz, 2H), 3.45 (q, J=5.6 Hz, 2H), 3.35 (s, 2H), 2.43 (t, J=5.3 Hz, 5H), 2.27-2.08 (m, 2H), 1.59 (q, J=6.1 Hz, 4H), 1.49-1.23 (m, 6H), 0.73-0.68 (m, 3H). MS (ESI) m/z 932.4 [M+H]⁺.

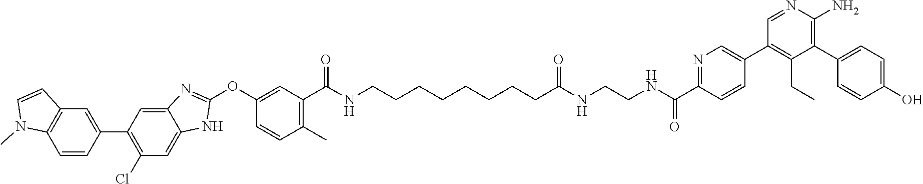 [SMILES: CCc1c(-c2ccc(C(=O)NCCNC(=O)CCCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 36: 6′-amino-N-(2-(9-(5-((6-chloro-5-(1-methyl-1H-indol-5-yl)-1H-benzo[d]imidazol-2-yl)oxy)-2-methylbenzamido)nonanamido)ethyl)-4′-ethyl-5′-(4-hydroxyphenyl)-[3,3′-bipyridine]-6-carboxamide (KL186-55)

Example 36 was synthesized following similar procedure for preparing example 29 from linker 8 and intermediate 6. Pink solid, 5.2 mg, 55% yield. ¹H NMR (400 MHz, Methanol-d₄) δ 8.67 (d, J=3.5 Hz, 1H), 8.24-8.17 (m, 1H), 8.02 (dd, J=6.9, 3.8 Hz, 1H), 7.78 (d, J=3.5 Hz, 1H), 7.56 (d, J=3.5 Hz, 1H), 7.49 (d, J=3.6 Hz, 1H), 7.44-7.31 (m, 5H), 7.27-7.20 (m, 2H), 7.20-7.14 (m, 2H), 7.02 (dt, J=8.4, 2.3 Hz, 2H), 6.48 (d, J=4.3 Hz, 1H), 3.86 (d, J=3.6 Hz, 3H), 3.56 (q, J=5.3 Hz, 2H), 3.46 (t, J=5.1 Hz, 2H), 3.37 (d, J=4.7 Hz, 2H), 2.45-2.39 (m, 5H), 2.16 (d, J=8.7 Hz, 2H), 1.59 (q, J=7.9, 6.4 Hz, 4H), 1.34 (d, J=34.3 Hz, 8H), 0.71 (q, J=6.7 Hz, 3H). MS (ESI) m/z 946.4 [M+H]⁺.

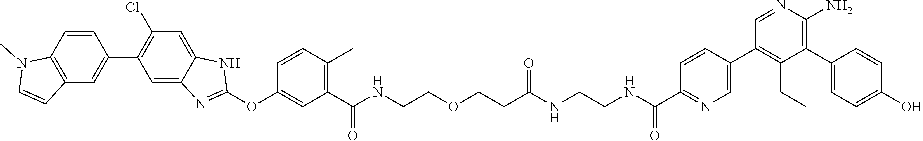 [SMILES: CCc1c(-c2ccc(C(=O)NCCNC(=O)CCOCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 37: 6′-amino-N-(2-(3-(2-(5-((6-chloro-5-(1-methyl-1H-indol-5-yl)-1H-benzo[d]imidazol-2-yl)oxy)-2-methylbenzamido)ethoxy)propanamido)ethyl)-4′-ethyl-5′-(4-hydroxyphenyl)-[3,3′-bipyridine]-6-carboxamide (KL186-56)

Example 37 was synthesized following similar procedure for preparing example 29 from linker 9 and intermediate 6. Pink solid, 5.3 mg, 59% yield. ¹H NMR (400 MHz, Methanol-d₄) δ 8.60 (d, J=3.2 Hz, 1H), 8.12 (dd, J=8.3, 3.4 Hz, 1H), 8.01-7.88 (m, 1H), 7.73 (d, J=2.8 Hz, 1H), 7.54 (d, J=3.4 Hz, 1H), 7.46 (dt, J=11.9, 2.5 Hz, 2H), 7.43-7.33 (m, 4H), 7.26-7.19 (m, 2H), 7.18-7.10 (m, 2H), 7.00 (dt, J=8.5, 2.3 Hz, 2H), 6.46 (d, J=4.6 Hz, 1H), 3.85 (t, J=2.5 Hz, 3H), 3.74 (q, J=5.4 Hz, 2H), 3.62 (t, J=4.9 Hz, 2H), 3.55-3.48 (m, 4H), 3.39 (d, J=5.2 Hz, 2H), 2.45 (d, J=3.7 Hz, 5H), 2.41-2.33 (m, 2H), 0.67 (q, J=6.8, 6.2 Hz, 3H). MS (ESI) m/z 906.3 [M+H]⁺.

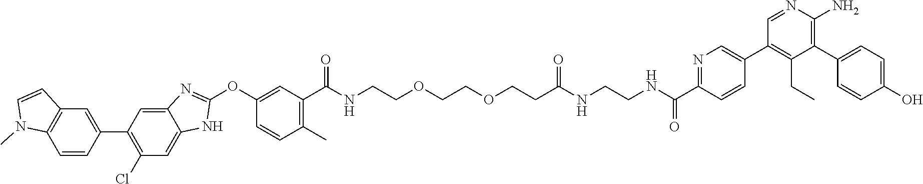 [SMILES: CCc1c(-c2ccc(C(=O)NCCNC(=O)CCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 38: 6′-amino-N-(1-(5-((6-chloro-5-(1-methyl-1H-indol-5-yl)-1H-benzo[d]imidazol-2-yl)oxy)-2-methylphenyl)-1,11-dioxo-5,8-dioxa-2,12-diazatetradecan-14-yl)-4′-ethyl-5′-(4-hydroxyphenyl)-[3,3′-bipyridine]-6-carboxamide (KL186-57)

Example 38 was synthesized following similar procedure for preparing example 29 from linker 10 and intermediate 6. Pink solid, 5.1 mg, 54% yield. ¹H NMR (400 MHz, Methanol-d₄) δ 8.63 (s, 1H), 8.15 (d, J=7.3 Hz, 1H), 8.02-7.91 (m, 1H), 7.75 (t, J=2.6 Hz, 1H), 7.55 (d, J=3.6 Hz, 1H), 7.50 (t, J=2.7 Hz, 1H), 7.47-7.31 (m, 6H), 7.22 (d, J=8.3 Hz, 2H), 7.15 (dd, J=7.7, 3.8 Hz, 2H), 7.04-6.97 (m, 2H), 6.47 (d, J=3.0 Hz, 1H), 3.86 (t, J=2.6 Hz, 3H), 3.67 (dt, J=10.8, 5.1 Hz, 4H), 3.62-3.52 (m, 8H), 3.43 (t, J=5.1 Hz, 2H), 2.45 (t, J=2.6 Hz, 3H), 2.40 (q, J=5.9 Hz, 4H), 0.69 (q, J=7.2, 5.4 Hz, 3H). MS (ESI) m/z 950.4 [M+H]⁻.

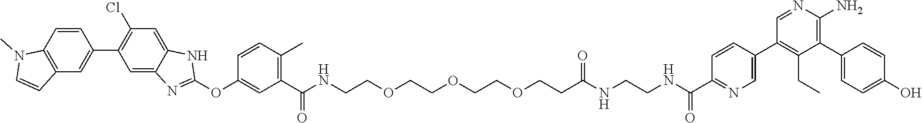 [SMILES: CCc1c(-c2ccc(C(=O)NCCNC(=O)CCOCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 39: 6′-amino-N-(1-(5-((6-chloro-5-(1-methyl-1H-indol-5-yl)-1H-benzo[d]imidazol-2-yl)oxy)-2-methylphenyl)-1,14-dioxo-5,8,11-trioxa-2,15-diazaheptadecan-17-yl)-4′-ethyl-5′-(4-hydroxyphenyl)-[3,3′-bipyridine]-6-carboxamide (KL186-58)

Example 39 was synthesized following similar procedure for preparing example 29 from linker 11 and intermediate 6. Pink solid, 5.9 mg, 60% yield. ¹H NMR (400 MHz, Methanol-d₄) δ 8.64 (d, J=3.3 Hz, 1H), 8.20-8.12 (m, 1H), 7.97 (d, J=8.1 Hz, 1H), 7.76 (t, J=2.8 Hz, 1H), 7.56 (d, J=3.5 Hz, 1H), 7.50 (d, J=3.0 Hz, 1H), 7.40 (dt, J=18.4, 5.6 Hz, 5H), 7.26-7.20 (m, 2H), 7.15 (dd, J=8.7, 3.0 Hz, 2H), 7.01 (dd, J=7.8, 3.3 Hz, 2H), 6.47 (q, J=2.9 Hz, 1H), 3.86 (t, J=2.6 Hz, 3H), 3.69-3.61 (m, 6H), 3.60-3.50 (m, 10H), 3.44 (d, J=5.0 Hz, 2H), 2.45 (t, J=2.7 Hz, 3H), 2.39 (q, J=9.4, 7.2 Hz, 4H), 0.71-0.62 (m, 3H). MS (ESI) m/z 994.4 [M+H]⁻.

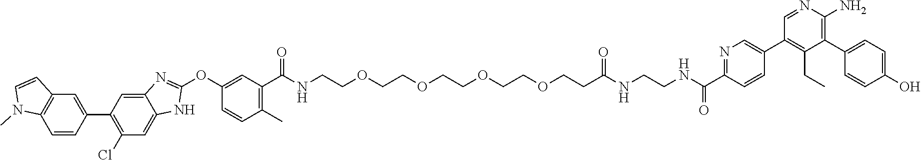 [SMILES: CCc1c(-c2ccc(C(=O)NCCNC(=O)CCOCCOCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 40: 6′-amino-N-(1-(5-((6-chloro-5-(1-methyl-1H-indol-5-yl)-1H-benzo[d]imidazol-2-yl)oxy)-2-methylphenyl)-1,17-dioxo-5,8,11,14-tetraoxa-2,18-diazaicosan-20-yl)-4′-ethyl-5′-(4-hydroxyphenyl)-[3,3′-bipyridine]-6-carboxamide (KL186-59)

Example 40 was synthesized following similar procedure for preparing example 29 from linker 12 and intermediate 6. Pink solid, 5.8 mg, 56% yield. ¹H NMR (400 MHz, Methanol-d₄) δ 8.62 (d, J=3.2 Hz, 1H), 8.20-8.10 (m, 1H), 7.96 (d, J=8.3 Hz, 1H), 7.74 (d, J=3.4 Hz, 1H), 7.53 (d, J=3.4 Hz, 1H), 7.48 (d, J=3.3 Hz, 1H), 7.42-7.32 (m, 5H), 7.25-7.17 (m, 2H), 7.17-7.10 (m, 2H), 7.04-6.96 (m, 2H), 6.45 (d, J=3.5 Hz, 1H), 3.83 (d, J=3.5 Hz, 3H), 3.65 (t, J=5.4 Hz, 4H), 3.62-3.56 (m, 4H), 3.54 (q, J=5.4 Hz, 6H), 3.49 (d, J=3.6 Hz, 6H), 3.43 (t, J=5.1 Hz, 2H), 2.43 (d, J=3.5 Hz, 3H), 2.42-2.34 (m, 4H), 0.67 (q, J=6.9 Hz, 3H). MS (ESI) m/z 1038.5 [M+H]⁺.

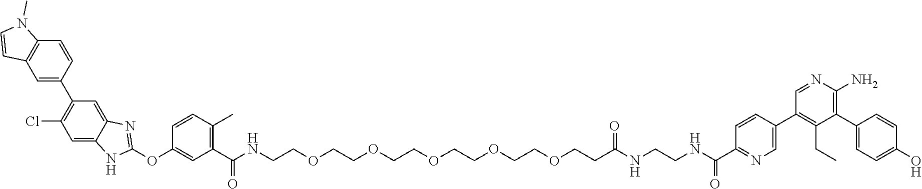 [SMILES: CCc1c(-c2ccc(C(=O)NCCNC(=O)CCOCCOCCOCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1]

### Example 41: 6′-amino-N-(1-(5-((6-chloro-5-(1-methyl-1H-indol-5-yl)-1H-benzo[d]imidazol-2-yl)oxy)-2-methylphenyl)-1,20-dioxo-5,8,11,14,17-pentaoxa-2,21-diazatricosan-23-yl)-4′-ethyl-5′-(4-hydroxyphenyl)-[3,3′-bipyridine]-6-carboxamide (KL186-60)

Example 41 was synthesized following similar procedure for preparing example 29 from linker 13 and intermediate 6. Pink solid, 5.8 mg, 54% yield. ¹H NMR (400 MHz, Methanol-d₄) δ 8.66 (d, J=3.3 Hz, 1H), 8.22-8.15 (m, 1H), 8.03-7.96 (m, 1H), 7.78 (t, J=2.7 Hz, 1H), 7.56 (d, J=3.5 Hz, 1H), 7.51 (t, J=2.7 Hz, 1H), 7.45-7.35 (m, 5H), 7.23 (d, J=11.6 Hz, 2H), 7.20-7.13 (m, 2H), 7.06-6.98 (m, 2H), 6.47 (q, J=2.9 Hz, 1H), 3.86 (t, J=2.6 Hz, 3H), 3.71-3.65 (m, 4H), 3.62 (q, J=5.8, 5.1 Hz, 4H), 3.58-3.51 (m, 16H), 3.46 (t, J=5.3 Hz, 2H), 2.48-2.45 (m, 3H), 2.42 (q, J=6.7, 5.7 Hz, 4H), 0.70 (q, J=6.8, 6.2 Hz, 3H). MS (ESI) m/z 1082.5 [M+H]⁺.

Procedure for the Synthesis of cGAS Based Bivalent Compounds

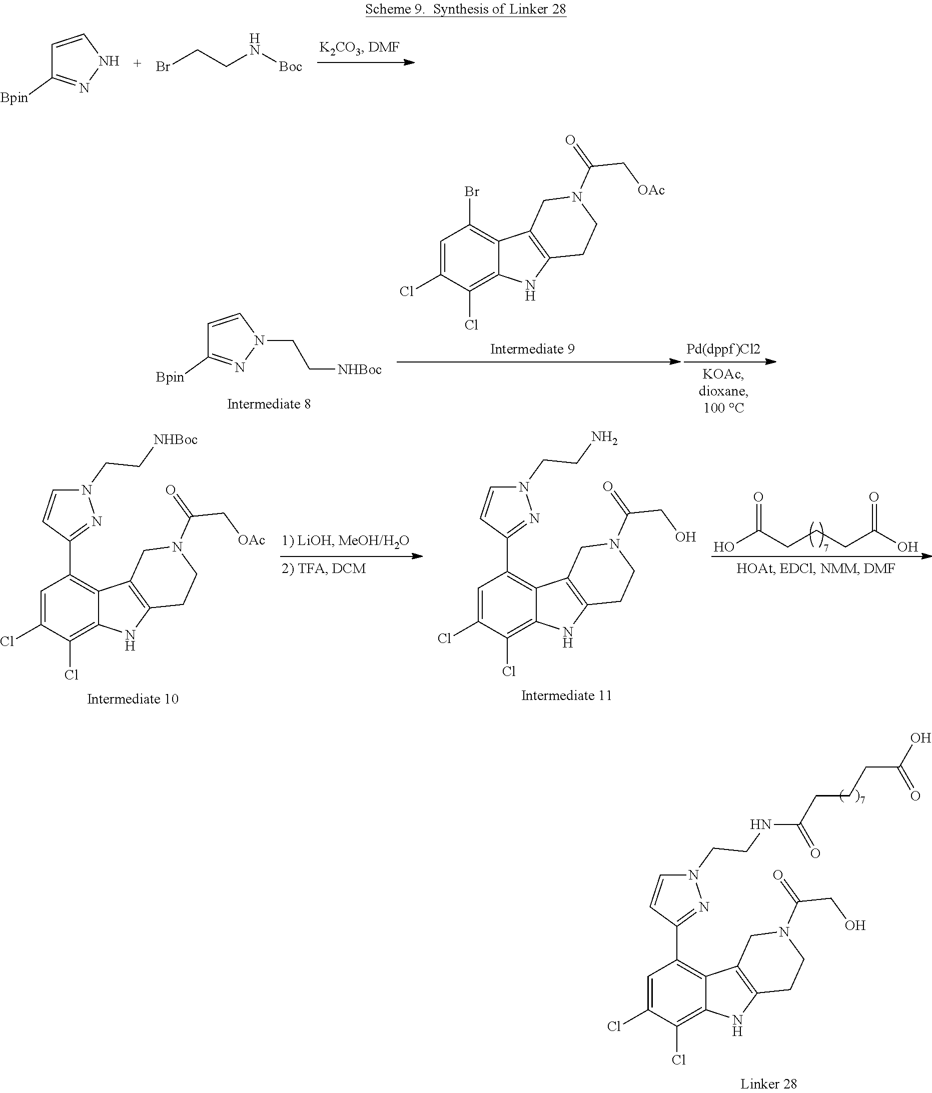

### Linker 28: 1-(9-(1-(2-aminoethyl)-1H-pyrazol-3-yl)-6,7-dichloro-1,3,4,5-tetrahydro-2H-pyrido[4,3-b]indol-2-yl)-2-hydroxyethan-1-one

To a solution of 3-(4,4,5,5-tetramethyl-1,3,2-dioxaborolan-2-yl)-1H-pyrazole (7.3 g, 37.6 mmol, 1.0 eq) in MeCN (30 ml) was added K₂CO₃ (15.6 g, 112.9 mmol, 3.0 eq) and tert-butyl(2-bromoethyl)carbamate (10.1 g, 45.1 mmol, 1.2 eq). The mixture was stirred at 80° C. for 6 h. After that, water was added and the mixture was extracted with ethyl acetate. The organic layer was collected, and excess solvent was removed to get intermediate 8 and used for the next step directly without further purification.

To a solution of intermediate 9 (1.05 g, 2.5 mmol, 1 eq) (prepared following previous reported lit.) (Lama et al., 2019) and intermediate 8 (3.2 g, 9.5 mmol, 3.8 eq) in dioxane (40 ml) were added KOAC (981 mg, 10.0 mmol, 4.0 eq) and Pd(dppf)Cl₂ (408 mg, 0.5 mmol, 0.2 eq). The mixture was stirred at 100° C. for 6 h under N₂ followed concentrated under reduced pressure. The resulted residue was purified by column chromatography (SiO₂, DCM/MeOH=10:1) to give intermediate 10 as a brown oil, yield 65%.

To a solution of intermediate 10 (420 mg, 0.76 mmol, 1 eq) in MeOH/H₂O (10:1, 4.4 ml) was added LiOH·H₂O (91 mg, 2.3 mmol, 3 eq). The mixture was stirred at rt for 10 min followed by concentrated and diluted with H₂O. After that, the pH value of the solution was adjusted to 6 with HCl aq. solution (1M) followed by concentration. The residue was re-dissolved in DCM/TFA (2:1, 7.5 mL). After stirring at rt for 1 h, excess solvent was removed and the residue was purified by prep-HPLC to give intermediate 11 as a yellow solid, yield 45%.

To a solution of undecanedioic acid (0.3 mmol, 2.0 eq) in dimethylformamide (0.75 mL) and DCM (18 mL) was added NMM (0.75 mmol, 5.0 eq), intermediate 11 (0.15 mmol, 1.0 eq), HOAt (0.18 mmol, 1.2 eq), and EDCI (0.18 mmol, 1.2 eq) at 0° C. The resulted reaction solution was stirred at 0° C. for 6 h before being stirred at room temperature overnight followed by concentrated. The resulted residue was purified by reverse-phase chromatography to yield the titled product, yield 50%. MS (ESI) [M+H]⁺=607.6.

Linker 29-42 were synthesized following the same procedure for preparing linker 28.

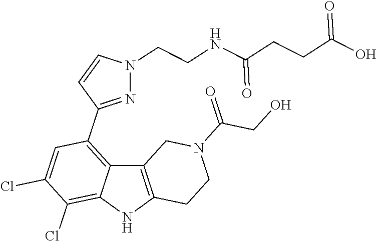 [SMILES: O=C(O)CCC(=O)NCCn1ccc(-c2cc(Cl)c(Cl)c3[nH]c4c(c23)CN(C(=O)CO)CC4)n1]

### Linker 29: 4-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-4-oxobutanoic acid

Brown solid, yield 48%. MS (ESI) [M+H]⁺=509.4.

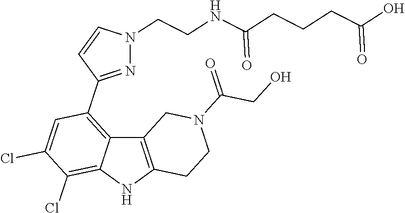 [SMILES: O=C(O)CCCC(=O)NCCn1ccc(-c2cc(Cl)c(Cl)c3[nH]c4c(c23)CN(C(=O)CO)CC4)n1]

### Linker 30: 5-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-5-oxopentanoic acid

Brown solid, yield 45%. MS (ESI) [M+H]⁺=523.3.

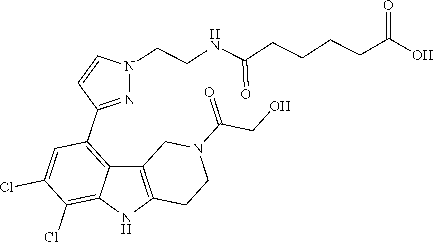 [SMILES: O=C(O)CCCCC(=O)NCCn1ccc(-c2cc(Cl)c(Cl)c3[nH]c4c(c23)CN(C(=O)CO)CC4)n1]

### Linker 31: 6-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-6-oxohexanoic acid

Brown solid, yield 47%. MS (ESI) [M+H]⁺=537.4.

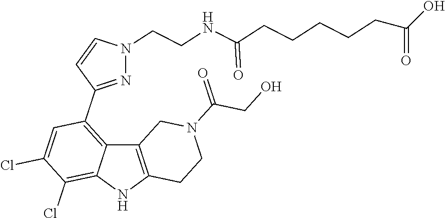 [SMILES: O=C(O)CCCCCC(=O)NCCn1ccc(-c2cc(Cl)c(Cl)c3[nH]c4c(c23)CN(C(=O)CO)CC4)n1]

### Linker 32: 7-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-7-oxoheptanoic acid

Brown solid, yield 53%. MS (ESI) [M+H]⁺=551.4.

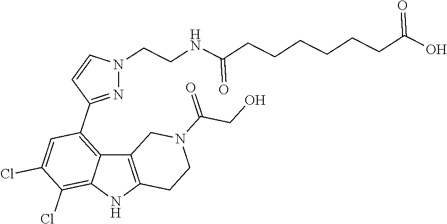 [SMILES: O=C(O)CCCCCCC(=O)NCCn1ccc(-c2cc(Cl)c(Cl)c3[nH]c4c(c23)CN(C(=O)CO)CC4)n1]

### Linker 33: 8-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-8-oxooctanoic acid

Brown solid, yield 58%. MS (ESI) [M+H]⁺=565.5.

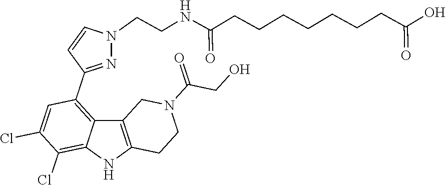 [SMILES: O=C(O)CCCCCCCC(=O)NCCn1ccc(-c2cc(Cl)c(Cl)c3[nH]c4c(c23)CN(C(=O)CO)CC4)n1]

### Linker 34: 9-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-9-oxononanoic acid

Brown solid, yield 56%. MS (ESI) [M+H]⁺=579.5.

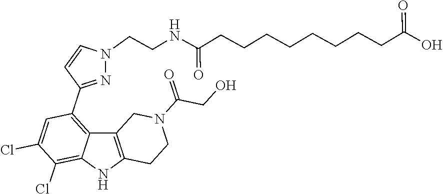 [SMILES: O=C(O)CCCCCCCCC(=O)NCCn1ccc(-c2cc(Cl)c(Cl)c3[nH]c4c(c23)CN(C(=O)CO)CC4)n1]

### Linker 35: 10-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-10-oxodecanoic acid

Brown solid, yield 60%. MS (ESI) [M+H]⁺=593.5.

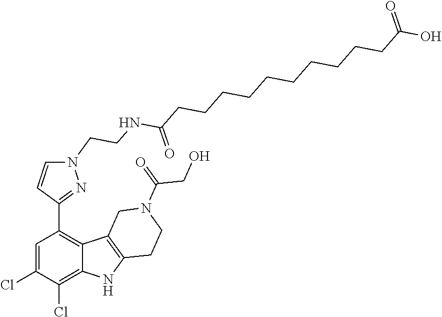 [SMILES: O=C(O)CCCCCCCCCCC(=O)NCCn1ccc(-c2cc(Cl)c(Cl)c3[nH]c4c(c23)CN(C(=O)CO)CC4)n1]

### Linker 36: 12-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-12-oxododecanoic acid

Brown solid, yield 45%. MS (ESI) [M+H]⁺=621.6.

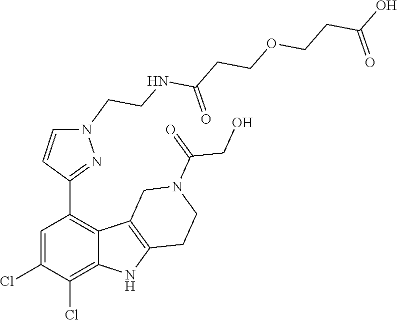 [SMILES: O=C(O)CCOCCC(=O)NCCn1ccc(-c2cc(Cl)c(Cl)c3[nH]c4c(c23)CN(C(=O)CO)CC4)n1]

### Linker 37: 3-(3-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-3-oxopropoxy)propanoic acid

Brown solid, yield 46%. MS (ESI) [M+H]⁺=553.4.

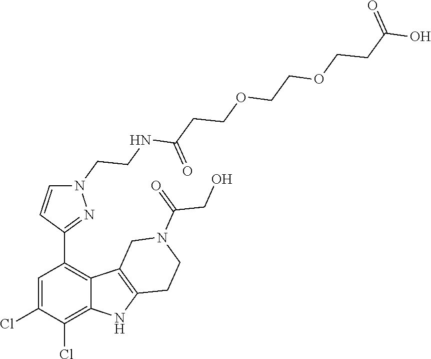 [SMILES: O=C(O)CCOCCOCCC(=O)NCCn1ccc(-c2cc(Cl)c(Cl)c3[nH]c4c(c23)CN(C(=O)CO)CC4)n1]

### Linker 38: 3-(2-(3-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-3-oxopropoxy)ethoxy)propanoic acid

Brown solid, yield 49%. MS (ESI) [M+H]⁺=597.5.

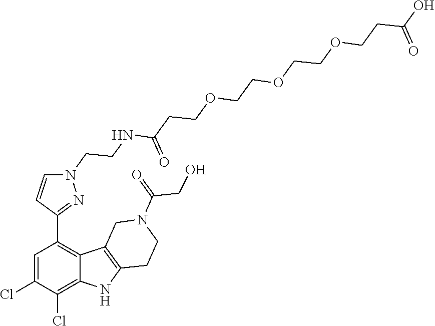 [SMILES: O=C(O)CCOCCOCCOCCC(=O)NCCn1ccc(-c2cc(Cl)c(Cl)c3[nH]c4c(c23)CN(C(=O)CO)CC4)n1]

### Linker 39: 1-(3-(6,7-dichloro-2-(2-hydroxyacetyl)-2,3,4,5-tetrahydro-1H-pyrido[4,3-b]indol-9-yl)-1H-pyrazol-1-yl)-4-oxo-7,10,13-trioxa-3-azahexadecan-16-oic acid

Brown solid, yield 52%. MS (ESI) [M+H]⁺=641.5.

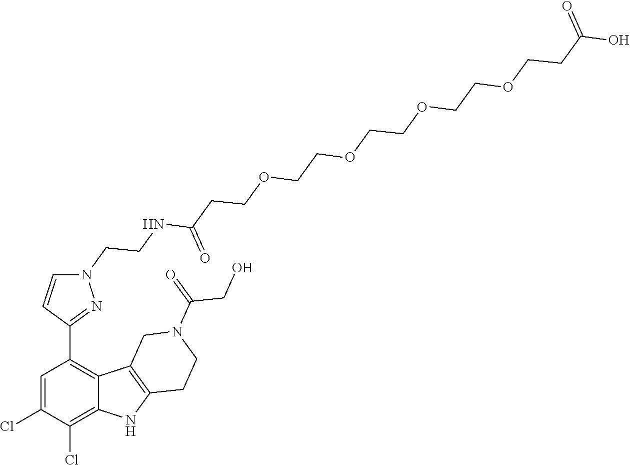 [SMILES: O=C(O)CCOCCOCCOCCOCCC(=O)NCCn1ccc(-c2cc(Cl)c(Cl)c3[nH]c4c(c23)CN(C(=O)CO)CC4)n1]

### Linker 40: 1-(3-(6,7-dichloro-2-(2-hydroxyacetyl)-2,3,4,5-tetrahydro-1H-pyrido[4,3-b]indol-9-yl)-1H-pyrazol-1-yl)-4-oxo-7,10,13,16-tetraoxa-3-azanonadecan-19-oic acid

Brown solid, yield 53%. MS (ESI) [M+H]⁺=685.6.

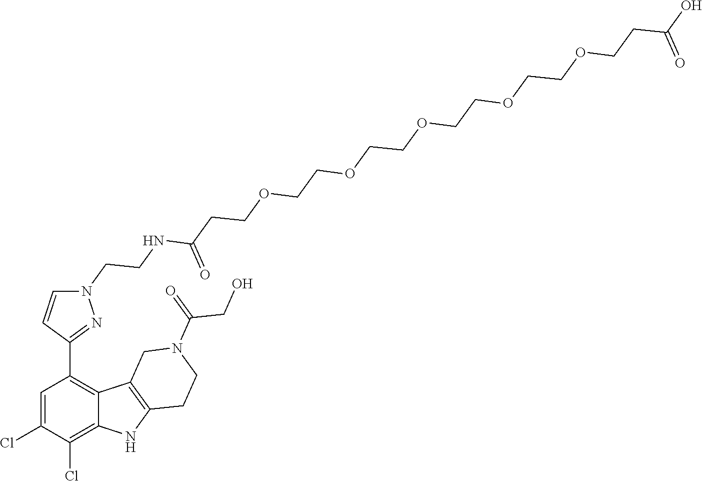 [SMILES: O=C(O)CCOCCOCCOCCOCCOCCC(=O)NCCn1ccc(-c2cc(Cl)c(Cl)c3[nH]c4c(c23)CN(C(=O)CO)CC4)n1]

### Linker 41: 1-(3-(6,7-dichloro-2-(2-hydroxyacetyl)-2,3,4,5-tetrahydro-1H-pyrido[4,3-b]indol-9-yl)-1H-pyrazol-1-yl)-4-oxo-7,10,13,16,19-pentaoxa-3-azadocosan-22-oic acid

Brown solid, yield 47%. MS (ESI) [M+H]⁺=729.6.

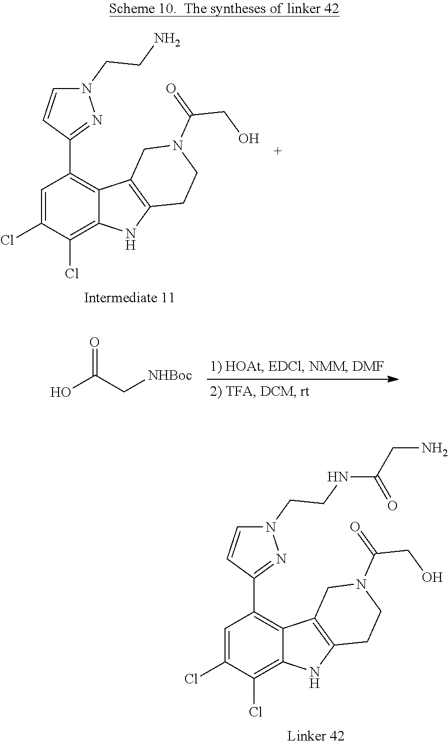 [SMILES: NCCn1ccc(-c2cc(Cl)c(Cl)c3[nH]c4c(c23)CN(C(=O)CO)CC4)n1 ; CC(C)(C)OC(=O)NCC(=O)O ; NCC(=O)NCCn1ccc(-c2cc(Cl)c(Cl)c3[nH]c4c(c23)CN(C(=O)CO)CC4)n1]

### Linker 42: 2-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)acetamide

To a solution of Boc-glycine (0.15 mmol, 1.5 eq) in DMF/DCM (1: 2, 3 mL) was added NMM (0.5 mmol, 5.0 eq), intermediate 11 (0.1 mmol, 1.0 eq), HOAt (0.12 mmol, 1.2 eq), and EDCI (0.12 mmol, 1.2 eq) at 0° C. The mixture was stirred at 0° C. for 6 h before being stirred at room temperature overnight followed by concentrated. The resulted residue was redissolved in DCM/TFA (2:1, 3 mL), and stirred at room temperature for 1 h followed by concentration. The residue was purified by reverse-phase chromatography to yield the titled product (Brown solid, 40% yield). MS (ESI) [M+H]⁺=466.3. Linker 43-56 were synthesized following the same procedure for preparing linker 42.

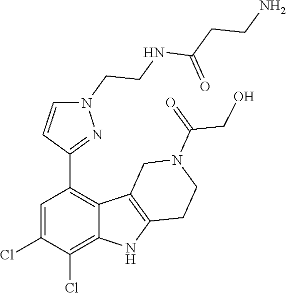 [SMILES: NCCC(=O)NCCn1ccc(-c2cc(Cl)c(Cl)c3[nH]c4c(c23)CN(C(=O)CO)CC4)n1]

### Linker 43: 3-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)propenamide

Brown solid, yield 42%. MS (ESI) [M+H]⁺=480.4.

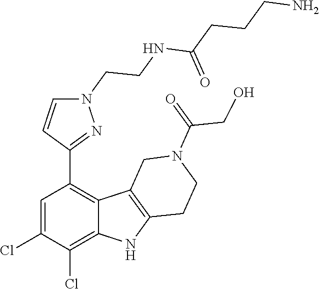 [SMILES: NCCCC(=O)NCCn1ccc(-c2cc(Cl)c(Cl)c3[nH]c4c(c23)CN(C(=O)CO)CC4)n1]

### Linker 44: 4-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)butanamide

Brown solid, yield 41%. MS (ESI) [M+H]⁺=494.4.

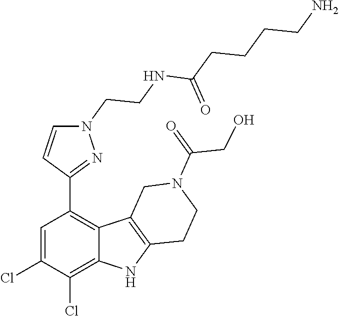 [SMILES: NCCCCC(=O)NCCn1ccc(-c2cc(Cl)c(Cl)c3[nH]c4c(c23)CN(C(=O)CO)CC4)n1]

### Linker 45: 5-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)pentanamide

Brown solid, yield 44%. MS (ESI) [M+H]⁺=508.4.

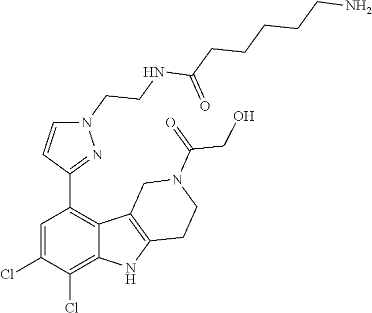 [SMILES: NCCCCCC(=O)NCCn1ccc(-c2cc(Cl)c(Cl)c3[nH]c4c(c23)CN(C(=O)CO)CC4)n1]

### Linker 46: 6-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)hexanamide

Brown solid, yield 47%. MS (ESI) [M+H]⁺=522.4.

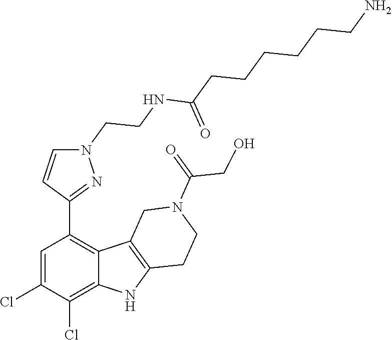 [SMILES: NCCCCCCC(=O)NCCn1ccc(-c2cc(Cl)c(Cl)c3[nH]c4c(c23)CN(C(=O)CO)CC4)n1]

### Linker 47: 7-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)heptanamide

Brown solid, yield 43%. MS (ESI) [M+H]⁺=536.5.

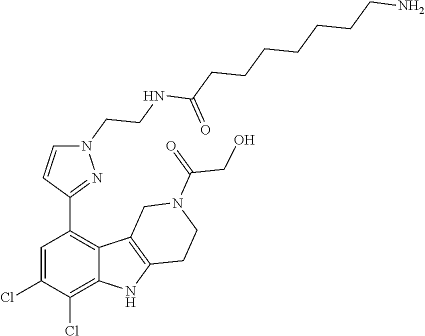 [SMILES: NCCCCCCCC(=O)NCCn1ccc(-c2cc(Cl)c(Cl)c3[nH]c4c(c23)CN(C(=O)CO)CC4)n1]

### Linker 48: 8-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)octanamide

Brown solid, yield 45%. MS (ESI) [M+H]⁺=550.5.

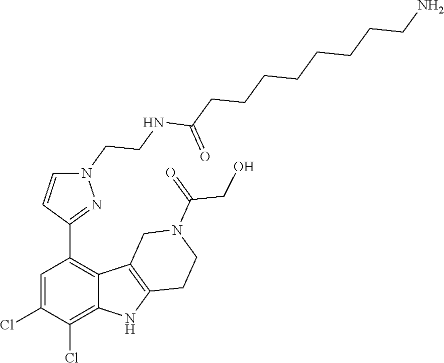 [SMILES: NCCCCCCCCC(=O)NCCn1ccc(-c2cc(Cl)c(Cl)c3[nH]c4c(c23)CN(C(=O)CO)CC4)n1]

### Linker 49: 9-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)nonanamide

Brown solid, yield 46%. MS (ESI) [M+H]⁺=564.5.

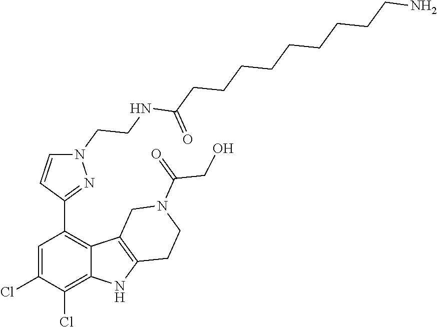 [SMILES: NCCCCCCCCCC(=O)NCCn1ccc(-c2cc(Cl)c(Cl)c3[nH]c4c(c23)CN(C(=O)CO)CC4)n1]

### Linker 50: 10-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)decanamide

Brown solid, yield 49%. MS (ESI) [M+H]⁺=578.6.

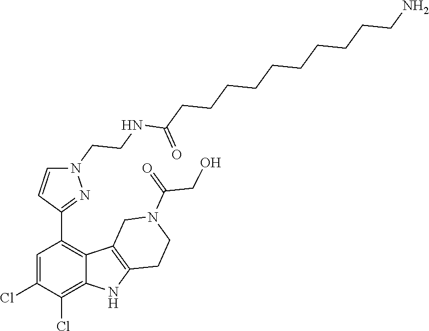 [SMILES: NCCCCCCCCCCC(=O)NCCn1ccc(-c2cc(Cl)c(Cl)c3[nH]c4c(c23)CN(C(=O)CO)CC4)n1]

### Linker 51: 11-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)undecanamide

Brown solid, yield 44%. MS (ESI) [M+H]⁺=592.6.

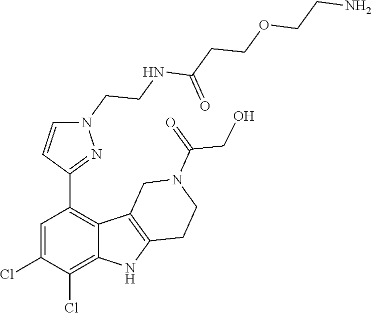 [SMILES: NCCOCCC(=O)NCCn1ccc(-c2cc(Cl)c(Cl)c3[nH]c4c(c23)CN(C(=O)CO)CC4)n1]

### Linker 52: 3-(2-aminoethoxy)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)propenamide

Brown solid, yield 42%. MS (ESI) [M+H]⁺=524.4.

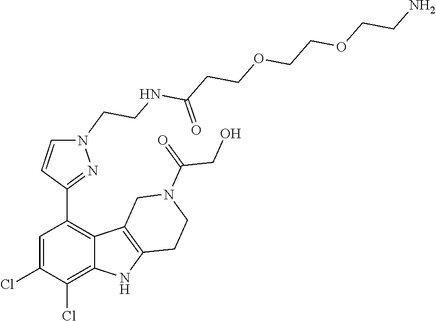 [SMILES: NCCOCCOCCC(=O)NCCn1ccc(-c2cc(Cl)c(Cl)c3[nH]c4c(c23)CN(C(=O)CO)CC4)n1]

### Linker 53: 3-(2-(2-aminoethoxy)ethoxy)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)propenamide

Brown solid, yield 45%. MS (ESI) [M+H]⁺=568.5.

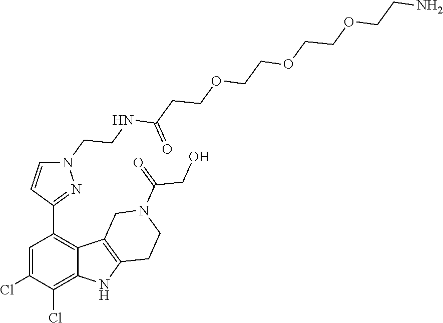 [SMILES: NCCOCCOCCOCCC(=O)NCCn1ccc(-c2cc(Cl)c(Cl)c3[nH]c4c(c23)CN(C(=O)CO)CC4)n1]

### Linker 54: 3-(2-(2-(2-aminoethoxy)ethoxy)ethoxy)-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)propenamide

Brown solid, yield 46%. MS (ESI) [M+H]⁺=612.5.

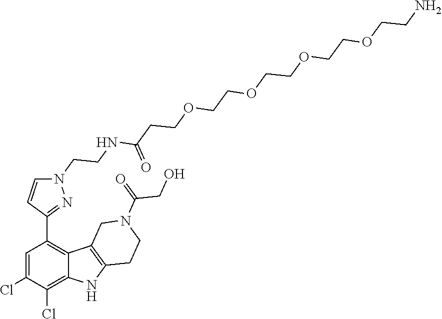 [SMILES: NCCOCCOCCOCCOCCC(=O)NCCn1ccc(-c2cc(Cl)c(Cl)c3[nH]c4c(c23)CN(C(=O)CO)CC4)n1]

### Linker 55: 1-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3,6,9,12-tetraoxapentadecan-15-amide

Brown solid, yield 42%. MS (ESI) [M+H]⁺=656.6.

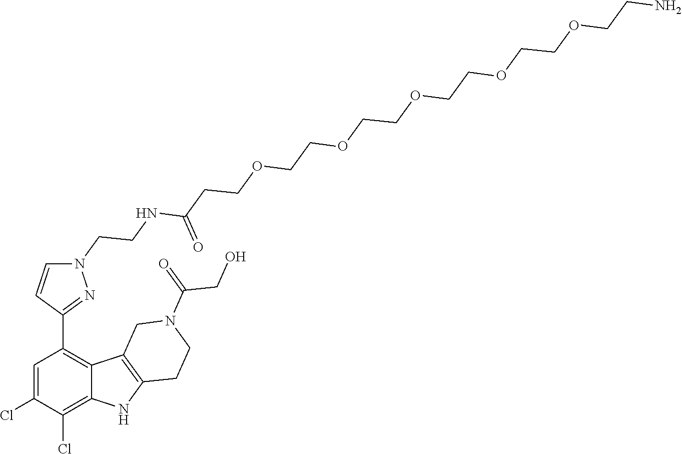 [SMILES: NCCOCCOCCOCCOCCOCCC(=O)NCCn1ccc(-c2cc(Cl)c(Cl)c3[nH]c4c(c23)CN(C(=O)CO)CC4)n1]

### Linker 56: 1-amino-N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3,6,9,12,15-pentaoxaoctadecan-18-amide

Brown solid, yield 44%. MS (ESI) [M+H]⁺=700.6.

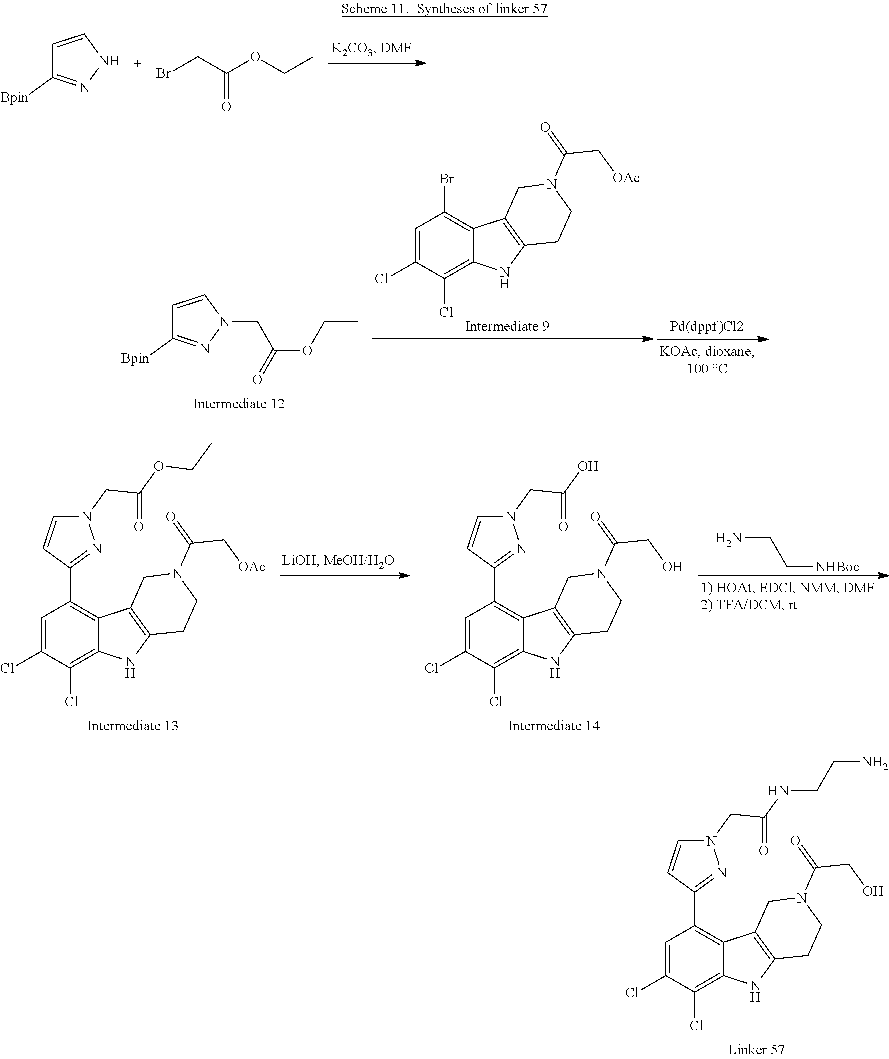

### Linker 57: N-(2-aminoethyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (XH168-108)

To a solution of 3-(4,4,5,5-tetramethyl-1,3,2-dioxaborolan-2-yl)-1H-pyrazole (7.3 g, 37.6 mmol, 1.0 eq) in MeCN (30 ml) was added K₂CO₃ (15.6 g, 112.9 mmol, 3.0 eq) and tert-butyl 2-bromoacetate (7.5 g, 45.1 mmol, 1.2 eq). The mixture was stirred at 80° C. for 6 h. After that, water was added and the mixture was extracted with ethyl acetate. The organic layer was collected, and excess solvent was removed to get intermediate 12 and used for the next step directly without further purification.

To a solution of intermediate 9 (1.05 g, 2.5 mmol, 1 eq) and intermediate 12 (2.7 g, 9.5 mmol, 3.8 eq) in dioxane (40 ml) was added KOAC (981.4 mg, 10.0 mmol, 4.0 eq) and Pd(dppf)Cl₂CH₂Cl₂ (408 mg, 0.5 mmol, 0.2 eq). The mixture was stirred at 100° C. for 6 h under N₂. Ater cooling down to room temperature, the reaction mixture was concentrated under reduced pressure and purified by column chromatography (SiO₂, DCM/MeOH=10:1) to give intermediate 13 as a brown oil, 68% yield.

To a solution of intermediate 13 (374.9 mg, 0.76 mmol, 1 eq) in MeOH (4 ml) and H₂O (0.4 ml) was added LiOH·H₂O (90.8 mg, 2.3 mmol, 3 eq). The mixture was stirred at rt for 10 min. Removed the MeOH and added H₂O, the PH of the residue was adjusted to 6 with HCl (1M). Remove the solvent and the residue was purified by prep-HPLC (acid condition) to give intermediate 14 as a yellow solid, 50% yield.

To a solution of intermediate 14 (65 mg, 0.12 mmol) in DMF (0.5 mL) were added tert-butyl(2-aminoethyl)carbamate (19.2 mg, 0.12 mmol, 1.0 equiv), 1-ethyl-3-(3-dimethylaminopropyl)carbodiimide (EDCI, 34.3 mg, 0.18 mmol, 1.5 equiv), 1-hydroxy-7-azabenzo-triazole (HOAt, 24.5 mg, 0.18 mmol, 1.5 equiv), and N-methylmorpholine (NMM, 36.4 mg, 0.36 mmol, 3.0 equiv). After being stirred for overnight at rt, the resulting mixture was purified by prep-HPLC to afford crude product. The crude product was dissolved in DCM/TFA (1:1, 2 mL) followed by stirred at rt for 30 min. The resulting mixture was purified by prep-HPLC to afford titled compound as yellow solid (44 mg, 63% yield). MS (ESI) [M+H]⁺=467.9. Linker 58-70 were synthesized following the same procedure for preparing linker 57.

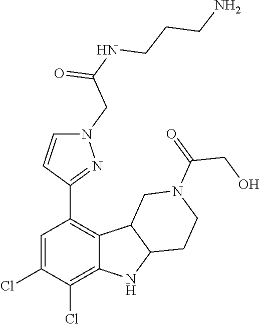 [SMILES: NCCCNC(=O)Cn1ccc(-c2cc(Cl)c(Cl)c3c2C2CN(C(=O)CO)CCC2N3)n1]

### Linker 58: N-(3-aminopropyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-109)

Yellow solid, 38 mg, 53% yield. MS (ESI) [M+H]⁺=479.2.

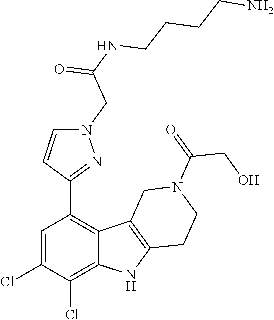 [SMILES: NCCCCNC(=O)Cn1ccc(-c2cc(Cl)c(Cl)c3[nH]c4c(c23)CN(C(=O)CO)CC4)n1]

### Linker 59: N-(4-aminobutyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-112)

Yellow solid, 27 mg, 37% yield. MS (ESI) [M+H]⁺=493.2.

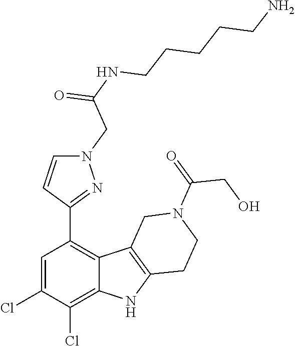 [SMILES: NCCCCCNC(=O)Cn1ccc(-c2cc(Cl)c(Cl)c3[nH]c4c(c23)CN(C(=O)CO)CC4)n1]

### Linker 60: N-(5-aminopentyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-113)

Example XH168-113 was synthesized following similar procedure for preparing example XH168-108. (yellow solid, 24 mg, yield 32%. MS (ESI) [M+H]⁺=507.3.

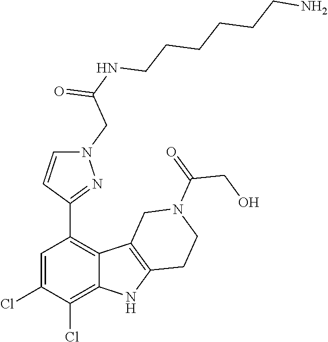 [SMILES: NCCCCCCNC(=O)Cn1ccc(-c2cc(Cl)c(Cl)c3[nH]c4c(c23)CN(C(=O)CO)CC4)n1]

### Linker 61: N-(6-aminohexyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-114)

Yellow solid, 29 mg, 38% yield. MS (ESI) [M+H]⁺=521.1.

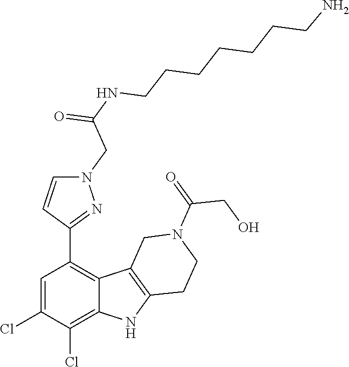 [SMILES: NCCCCCCCNC(=O)Cn1ccc(-c2cc(Cl)c(Cl)c3[nH]c4c(c23)CN(C(=O)CO)CC4)n1]

### Linker 62: N-(7-aminoheptyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-115)

Yellow solid, 27 mg, 35% yield. MS (ESI) [M+H]⁺=535.1.

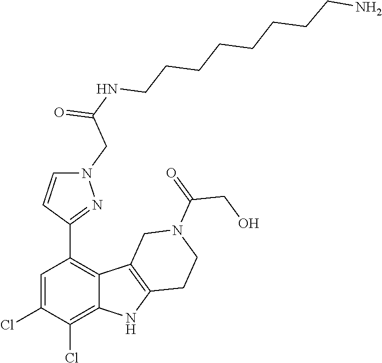 [SMILES: NCCCCCCCCNC(=O)Cn1ccc(-c2cc(Cl)c(Cl)c3[nH]c4c(c23)CN(C(=O)CO)CC4)n1]

### Linker 63: N-(8-aminooctyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-116)

Yellow solid, 26 mg, 33% yield. MS (ESI) [M+H]⁺=549.3.

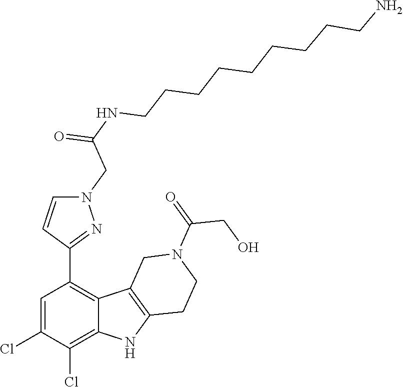 [SMILES: NCCCCCCCCCNC(=O)Cn1ccc(-c2cc(Cl)c(Cl)c3[nH]c4c(c23)CN(C(=O)CO)CC4)n1]

### Linker 64: N-(9-aminononyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-117)

Yellow solid, 64 mg, 79% yield. MS (ESI) [M+H]⁺=563.4.

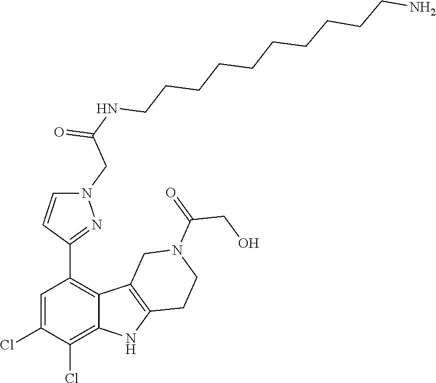 [SMILES: NCCCCCCCCCCNC(=O)Cn1ccc(-c2cc(Cl)c(Cl)c3[nH]c4c(c23)CN(C(=O)CO)CC4)n1]

### Linker 65: N-(10-aminodecyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-118)

Yellow solid, 39 mg, 47% yield. MS (ESI) [M+H]⁺=577.3.

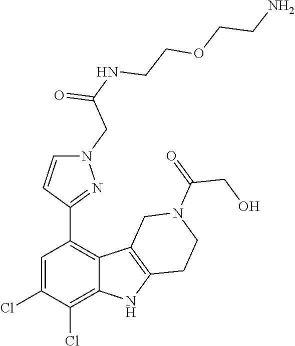 [SMILES: NCCOCCNC(=O)Cn1ccc(-c2cc(Cl)c(Cl)c3[nH]c4c(c23)CN(C(=O)CO)CC4)n1]

### Linker 66: N-(2-(2-aminoethoxy)ethyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-119)

Yellow solid, 40 mg, 53% yield. MS (ESI) [M+H]⁺=509.3.

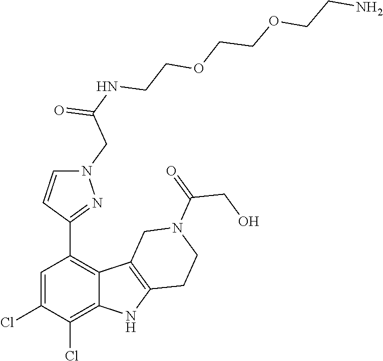 [SMILES: NCCOCCOCCNC(=O)Cn1ccc(-c2cc(Cl)c(Cl)c3[nH]c4c(c23)CN(C(=O)CO)CC4)n1]

### Linker 67: N-(2-(2-(2-aminoethoxy)ethoxy)ethyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-120)

Yellow solid, 61 mg, 46% yield. MS (ESI) [M+H]⁺=555.1.

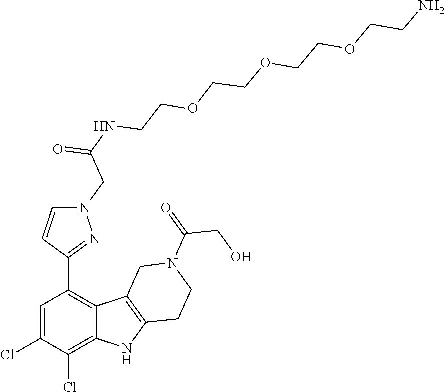 [SMILES: NCCOCCOCCOCCNC(=O)Cn1ccc(-c2cc(Cl)c(Cl)c3[nH]c4c(c23)CN(C(=O)CO)CC4)n1]

### Linker 68: N-(2-(2-(2-(2-aminoethoxy)ethoxy)ethoxy)ethyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-121)

Yellow solid, 43 mg, 30% yield. MS (ESI [M+H]⁺=597.5.

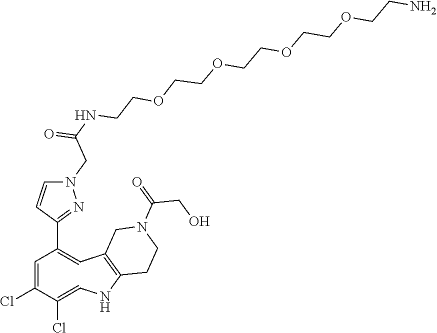 [SMILES: NCCOCCOCCOCCOCCNC(=O)Cn1ccc(-c2cc(Cl)c(Cl)c[nH]c3c(c2)CN(C(=O)CO)CC3)n1]

### Linker 69: N-(14-amino-3,6,9,12-tetraoxatetradecyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-122)

Yellow solid, 26 mg, 29% yield. MS (ESI) [M+H]⁺=641.3.

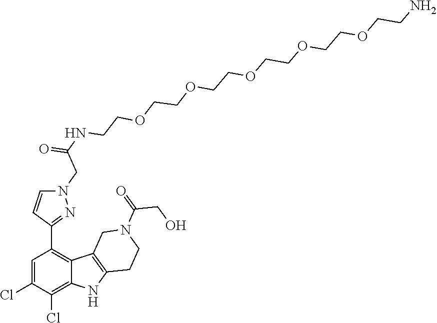 [SMILES: NCCOCCOCCOCCOCCOCCNC(=O)Cn1ccc(-c2cc(Cl)c(Cl)c3[nH]c4c(c23)CN(C(=O)CO)CC4)n1]

### Linker 70: N-(17-amino-3,6,9,12,15-pentaoxaheptadecyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (xh168-123)

Yellow solid, 31 mg, 32% yield. MS (ESI) [M+H]⁺=685.3.

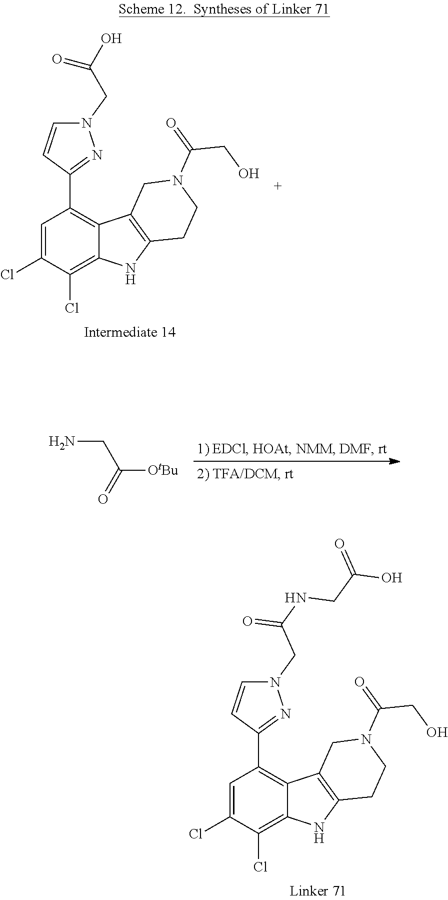 [SMILES: O=C(O)Cn1ccc(-c2cc(Cl)c(Cl)c3[nH]c4c(c23)CN(C(=O)CO)CC4)n1 ; CC(C)(C)OC(=O)CN ; O=C(O)CNC(=O)Cn1ccc(-c2cc(Cl)c(Cl)c3[nH]c4c(c23)CN(C(=O)CO)CC4)n1]

### Linker 71: N-(2-aminoethyl)-2-(3-(6,7-dichloro-2-(2-hydroxyacetyl)-2,3,4,5-tetrahydro-1H-pyrido[4,3-b]indol-9-yl)-1H-pyrazol-1-yl)acetamide (XH168-108)

To a solution of Intermediate 14 (50 mg, 0.1 mmol) in DMF (1 mL) were added tert-butyl glycinate (13.1 mg, 0.1 mmol, 1.0 equiv), 1-ethyl-3-(3-dimethylaminopropyl)carbodiimide (EDCI, 28.7 mg, 0.15 mmol, 1.5 equiv), 1-hydroxy-7-azabenzo-triazole (HOAt, 20.4 mg, 0.15 mmol, 1.5 equiv), and N-methylmorpholine (NMM, 30.3 mg, 0.3 mmol, 3.0 equiv). After being stirred at rt overnight, the resulting mixture was purified by prep-HPLC to afford crude product. The crude product was dissolved in DCM/TFA (1:1, 2 mL) followed by stirred at rt for 30 min. The resulting mixture was purified by prep-HPLC to afford titled compound as white solid (16 mg, 32% yield). MS (ESI) [M+H]⁺=480.3.

Linkers 72-83 were synthesized following the same procedure for preparing linker 71.

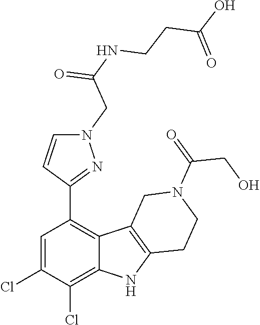 [SMILES: O=C(O)CCNC(=O)Cn1ccc(-c2cc(Cl)c(Cl)c3[nH]c4c(c23)CN(C(=O)CO)CC4)n1]

### Linker 72: 3-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)propanoic acid (XH181-71)

White solid, 24 mg, 49% yield. MS (ESI) [M+H]⁺=494.2.

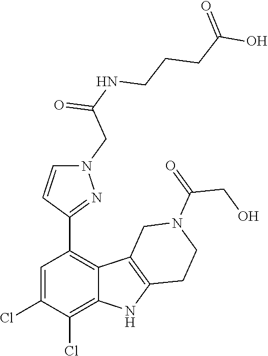 [SMILES: O=C(O)CCCNC(=O)Cn1ccc(-c2cc(Cl)c(Cl)c3[nH]c4c(c23)CN(C(=O)CO)CC4)n1]

### Linker 73: 4-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)butanoic acid (XH181-74)

White solid, 20 mg, 70% yield. MS (ESI) [M+H]⁺=508.11.

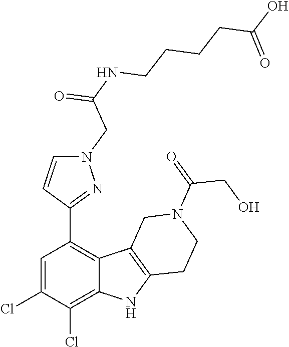 [SMILES: O=C(O)CCCCNC(=O)Cn1ccc(-c2cc(Cl)c(Cl)c3[nH]c4c(c23)CN(C(=O)CO)CC4)n1]

### Linker 74: 5-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)pentanoic acid (XH181-75)

White solid, 14 mg, 47% yield. MS (ESI) [M+H]⁺=522.2.

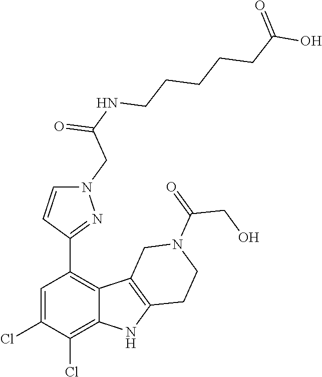 [SMILES: O=C(O)CCCCCNC(=O)Cn1ccc(-c2cc(Cl)c(Cl)c3[nH]c4c(c23)CN(C(=O)CO)CC4)n1]

### Linker 75: 6-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)hexanoic acid (XH181-151)

Yellow solid, 16 mg, 25% yield. MS (ESI) [M+H]⁺=536.3.

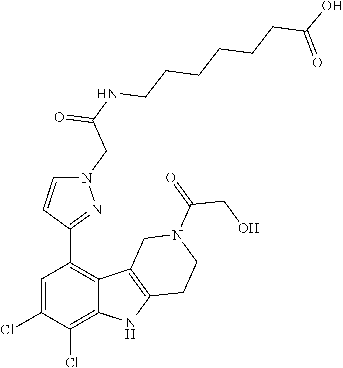 [SMILES: O=C(O)CCCCCCNC(=O)Cn1ccc(-c2cc(Cl)c(Cl)c3[nH]c4c(c23)CN(C(=O)CO)CC4)n1]

### Linker 76: 7-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)heptanoic acid (XH181-152)

Yellow solid, 18 mg, 27% yield. MS (ESI) [M+H]⁺=550.6.

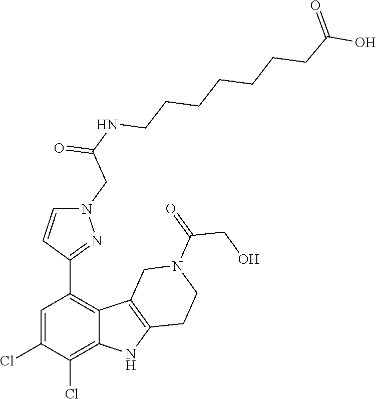 [SMILES: O=C(O)CCCCCCCNC(=O)Cn1ccc(-c2cc(Cl)c(Cl)c3[nH]c4c(c23)CN(C(=O)CO)CC4)n1]

### Linker 77: 8-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)octanoic acid (XH181-81)

White solid, 46 mg, 67% yield. MS (ESI) [M+H]⁺=564.7.

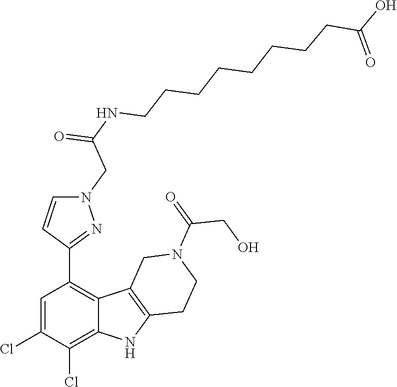 [SMILES: O=C(O)CCCCCCCCNC(=O)Cn1ccc(-c2cc(Cl)c(Cl)c3[nH]c4c(c23)CN(C(=O)CO)CC4)n1]

### Linker 78: 9-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)nonanoic acid (XH181-82)

White solid, 38 mg, 54% yield. MS (ESI) [M+H]⁺=578.6.

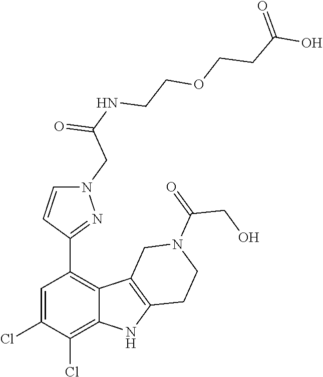 [SMILES: O=C(O)CCOCCNC(=O)Cn1ccc(-c2cc(Cl)c(Cl)c3[nH]c4c(c23)CN(C(=O)CO)CC4)n1]

### Linker 79: 3-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)propanoic acid (XH181-153)

White solid, 23 mg, 35% yield). MS (ESI) [M+H]⁺538.1.

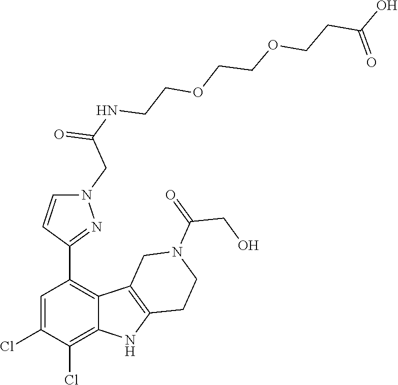 [SMILES: O=C(O)CCOCCOCCNC(=O)Cn1ccc(-c2cc(Cl)c(Cl)c3[nH]c4c(c23)CN(C(=O)CO)CC4)n1]

### Linker 80: 3-(2-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)ethoxy)propanoic acid (XH181-154)

Yellow solid, 33 mg, 47% yield. MS (ESI) [M+H]⁺=582.6.

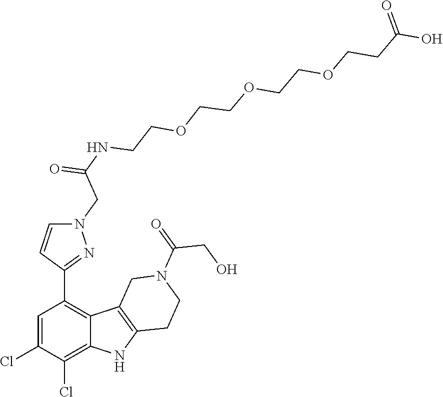 [SMILES: O=C(O)CCOCCOCCOCCNC(=O)Cn1ccc(-c2cc(Cl)c(Cl)c3[nH]c4c(c23)CN(C(=O)CO)CC4)n1]

### Linker 81: 1-(3-(6,7-dichloro-2-(2-hydroxyacetyl)-2,3,4,5-tetrahydro-1H-pyrido[4,3-b]indol-9-yl)-1H-pyrazol-1-yl)-2-oxo-6,9,12-trioxa-3-azapentadecan-15-oic acid (XH181-155)

Yellow solid, 54 mg, 71% yield. MS (ESI) [M+H]⁺=626.7.

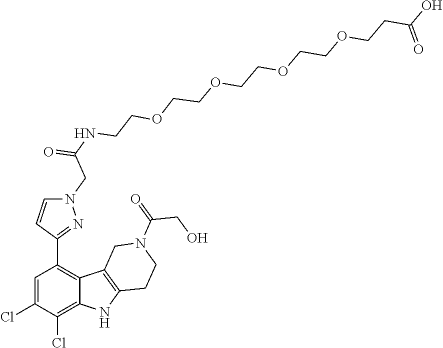 [SMILES: O=C(O)CCOCCOCCOCCOCCNC(=O)Cn1ccc(-c2cc(Cl)c(Cl)c3[nH]c4c(c23)CN(C(=O)CO)CC4)n1]

### Linker 82: 1-(3-(6,7-dichloro-2-(2-hydroxyacetyl)-2,3,4,5-tetrahydro-1H-pyrido[4,3-b]indol-9-yl)-1H-pyrazol-1-yl)-2-oxo-6,9,12,15-tetraoxa-3-azaoctadecan-18-oic acid (XH181-156)

Yellow solid, 49 mg, 61% yield. MS (ESI) [M+H]⁺=670.5.

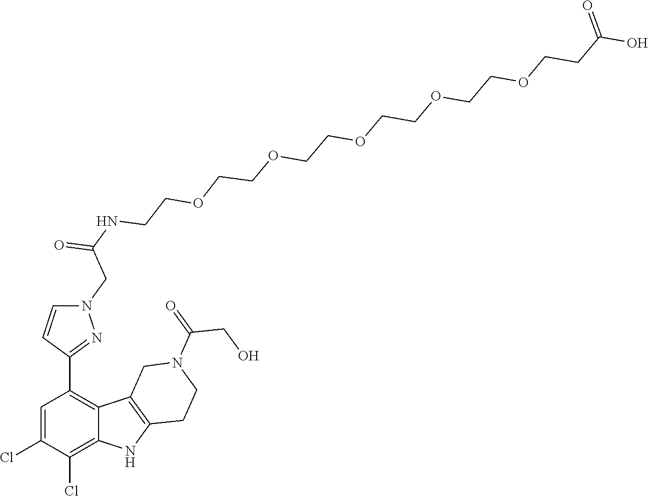 [SMILES: O=C(O)CCOCCOCCOCCOCCOCCNC(=O)Cn1ccc(-c2cc(Cl)c(Cl)c3[nH]c4c(c23)CN(C(=O)CO)CC4)n1]

### Linker 83: 1-(3-(6,7-dichloro-2-(2-hydroxyacetyl)-2,3,4,5-tetrahydro-1H-pyrido[4,3-b]indol-9-yl)-1H-pyrazol-1-yl)-2-oxo-6,9,12,15,18-pentaoxa-3-azahenicosan-21-oic acid (XH181-157)

Yellow solid, 54 mg, 63% yield. MS (ESI) [M+H]⁺=714.1.

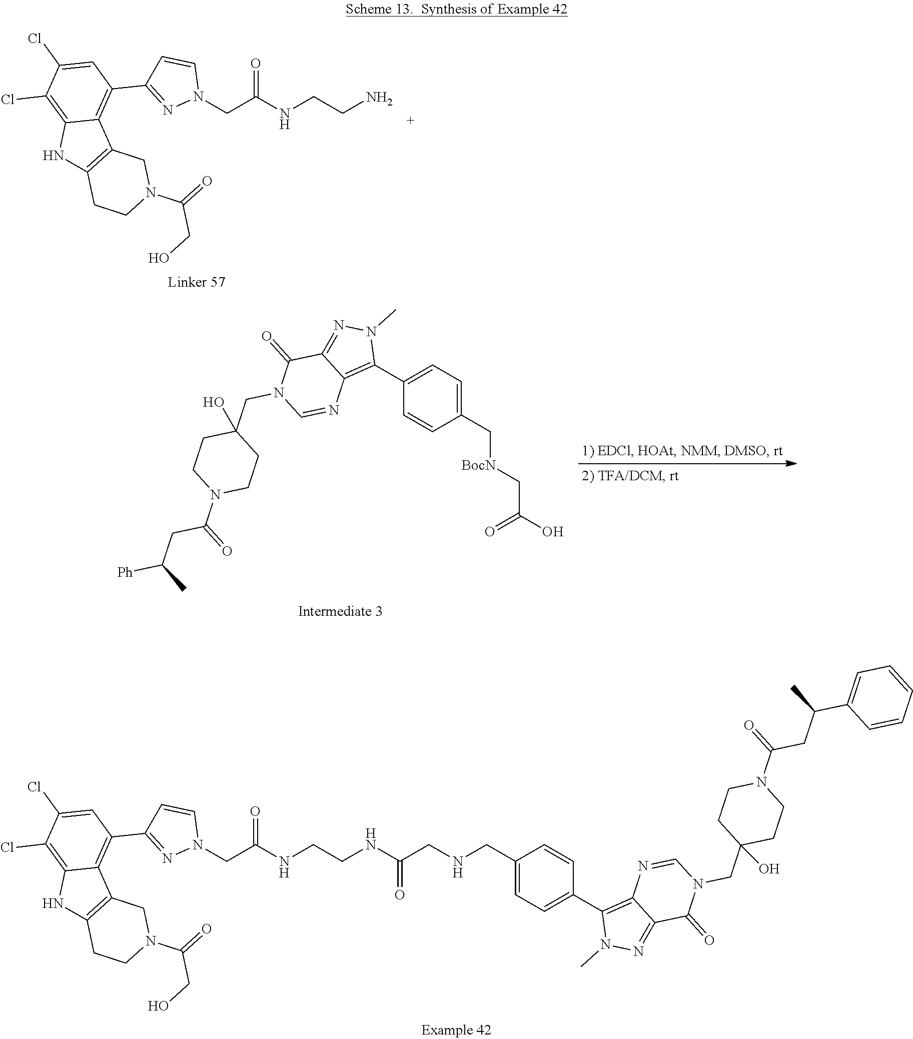 [SMILES: NCCNC(=O)Cn1ccc(-c2cc(Cl)c(Cl)c3[nH]c4c(c23)CN(C(=O)CO)CC4)n1 ; C[C@H](CC(=O)N1CCC(O)(Cn2cnc3c(-c4ccc(CN(CC(=O)O)C(=O)OC(C)(C)C)cc4)n(C)nc3c2=O)CC1)c1ccccc1 ; C[C@H](CC(=O)N1CCC(O)(Cn2cnc3c(-c4ccc(CNCC(=O)NCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 42: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(2-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)ethyl)acetamide (XH168-142)

To a solution of linker 57 (10.0 mg, 0.015 mmol) in DMF (0.5 mL) were added intermediate 3 (8.7 mg, 0.015 mmol, 1.0 equiv), 1-ethyl-3-(3-dimethylaminopropyl)carbodiimide (EDCI, 4.3 mg, 0.0225 mmol, 1.5 equiv), 1-hydroxy-7-azabenzo-triazole (HOAt, 3.1 mg, 0.0225 mmol, 1.5 equiv), and N-methylmorpholine (NMM, 4.5 mg, 0.045 mmol, 3.0 equiv). After being stirred for overnight at rt, the resulting mixture was purified by preparative HPLC to afford crude product. The crude product was dissolved in MeOH (0.5 mL) and HCl in dioxane (4 M, 0.5 mL). After being stirred at rt for 30 min, the resulting mixture was purified by preparative HPLC to afford titled compound as white solid (5.9 mg, yield 35%). ¹H NMR (400 MHz, Methanol-d₄) δ 7.97-7.92 (m, 1H), 7.77 (s, 1H), 7.67-7.65 (m, 1H), 7.62-7.58 (m, 2H), 7.50-7.47 (m, 1H), 7.32-7.17 (m, 6H), 6.53 (s, 1H), 4.98 (s, 2H), 4.65 (s, 1H), 4.40 (s, 1H), 4.34 (s, 1H), 4.24-4.19 (m, 2H), 4.00-4.06 (m, 4H), 4.02-3.81 (m, 3H), 3.75-3.61 (m, 4H), 3.40-3.34 (m, 5H), 3.27-3.14 (m, 2H), 3.05-2.84 (m, 3H), 2.78-2.70 (m, 1H), 2.62-2.45 (m, 1H), 1.65-1.55 (m, 1H), 1.48-1.28 (m, 6H). MS (ESI) [M+H]⁺=1019.5.

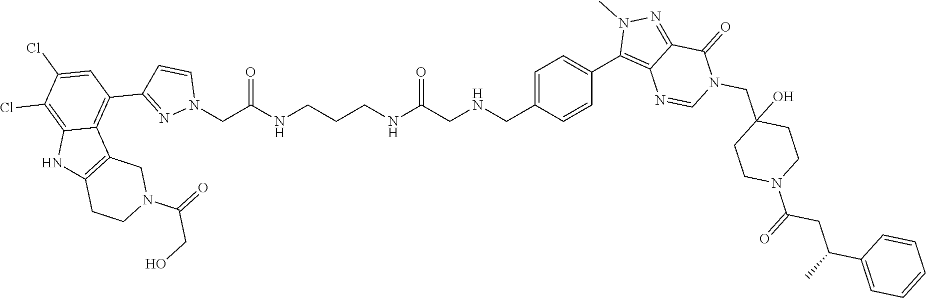 [SMILES: C[C@H](CC(=O)N1CCC(O)(Cn2cnc3c(-c4ccc(CNCC(=O)NCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 43: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(3-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)propyl)acetamide (XH168-143)

Example 43 was synthesized following the same procedure for preparing example 42 from linker 58 and intermediate 3. (white solid, 3.9 mg, yield 23%). ¹H NMR (400 MHz, Methanol-d₄) δ 8.00-7.90 (m, 1H), 7.78-7.67 (m, 4H), 7.58-7.52 (m, 2H), 7.32-7.17 (m, 6H), 6.55 (m, 1H), 4.94 (s, 2H), 4.64 (s, 1H), 4.37-4.32 (m, 2H), 4.24-4.17 (m, 2H), 4.09-4.06 (m, 4H), 4.01-3.81 (m, 4H), 3.69-3.60 (m, 3H), 3.23-3.16 (m, 6H), 3.05-2.84 (m, 3H), 2.78-2.69 (m, 1H), 2.61-2.56 (m, 1H), 2.49-2.46 (m, 1H), 1.73-1.71 (m, 3H), 1.58-1.28 (m, 6H). MS (ESI) [M+H]⁺=1033.5.

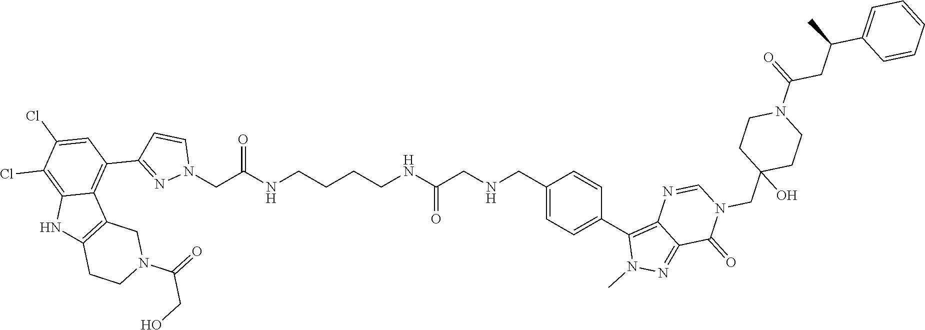 [SMILES: C[C@H](CC(=O)N1CCC(O)(Cn2cnc3c(-c4ccc(CNCC(=O)NCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 44: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(4-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)butyl)acetamide (XH168-144)

Example 44 was synthesized following the same procedure for preparing example 42 from linker 59 and intermediate 3. (white solid, 6.9 mg, yield 40%). ¹H NMR (400 MHz, Methanol-d₄) δ 7.98-7.88 (m, 1H), 7.75-7.56 (m, 5H), 7.32-7.14 (m, 6H), 6.55-6.52 (m, 1H), 4.94-4.92 (m, 2H), 4.62 (s, 1H), 4.43 (s, 1H), 4.33 (s, 2H), 4.26-4.18 (m, 3H), 4.11-4.00 (m, 4H), 3.93-3.89 (m, 2H), 3.82-3.76 (m, 2H), 3.69-3.59 (m, 2H), 3.34 (s, 1H), 3.26-3.14 (m, 6H), 3.04-2.84 (m, 3H), 2.80-2.69 (m, 1H), 2.61-2.44 (m, 1H), 1.56-1.47 (m, 5H), 1.42-1.26 (m, 6H). MS (ESI) [M+H]⁺=1047.5.

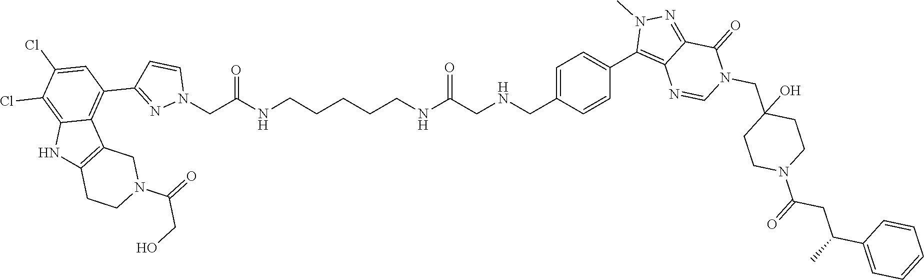 [SMILES: C[C@H](CC(=O)N1CCC(O)(Cn2cnc3c(-c4ccc(CNCC(=O)NCCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 45: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(5-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)pentyl)acetamide (XH168-145)

Example 45 was synthesized following the same procedure for preparing example 42 from linker 60 and intermediate 3. (white solid, 8.1 mg, yield 46%). ¹H NMR (400 MHz, Methanol-d₄) δ 7.98-7.89 (m, 1H), 7.75-7.56 (m, 5H), 7.32-7.15 (m, 6H), 6.55 (s, 1H), 4.93 (s, 2H), 4.65 (s, 1H), 4.39 (s, 1H), 4.32 (s, 2H), 4.24-4.19 (m, 2H), 4.11-4.00 (m, 4H), 3.93-3.78 (m, 4H), 3.71-3.59 (m, 2H), 3.34 (s, 1H), 3.23-3.21 (m, 6H), 3.04-2.91 (m, 3H), 2.84-2.68 (m, 1H), 2.61-2.44 (m, 1H), 1.60-1.52 (m, 5H), 1.41-1.30 (m, 8H). MS (ESI) [M+H]⁺=1161.5.

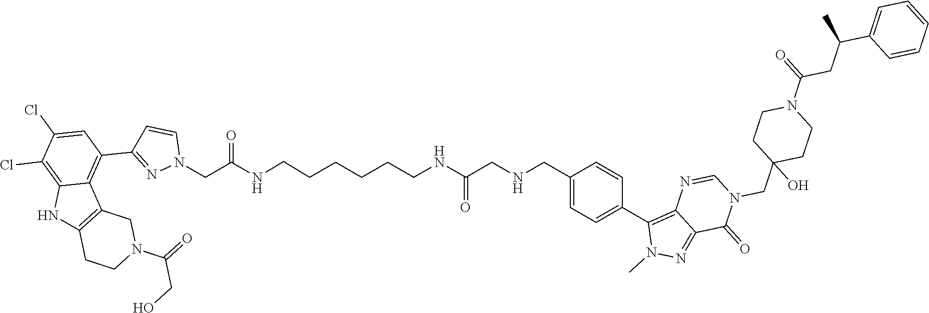 [SMILES: C[C@H](CC(=O)N1CCC(O)(Cn2cnc3c(-c4ccc(CNCC(=O)NCCCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 46: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(6-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)hexyl)acetamide (XH168-146)

Example 46 was synthesized following the same procedure for preparing example 42 from linker 61 and intermediate 3. (white solid, 7.1 mg, yield 40%). ¹H NMR (400 MHz, Methanol-d₄) δ 7.97-7.85 (m, 1H), 7.74-7.59 (m, 5H), 7.30-7.17 (m, 6H), 6.55-6.52 (m, 1H), 4.92 (s, 2H), 4.63 (s, 1H), 4.36 (s, 1H), 4.31 (s, 2H), 4.26-4.15 (m, 2H), 4.08-4.00 (m, 4H), 3.93-3.80 (m, 4H), 3.69-3.58 (m, 2H), 3.24-3.13 (m, 6H), 3.04-2.80 (m, 3H), 2.76-2.68 (m, 1H), 2.60-2.43 (m, 1H), 1.60-1.49 (m, 6H), 1.38-1.29 (m, 10H).). MS (ESI) [M+H]⁺=1075.5.

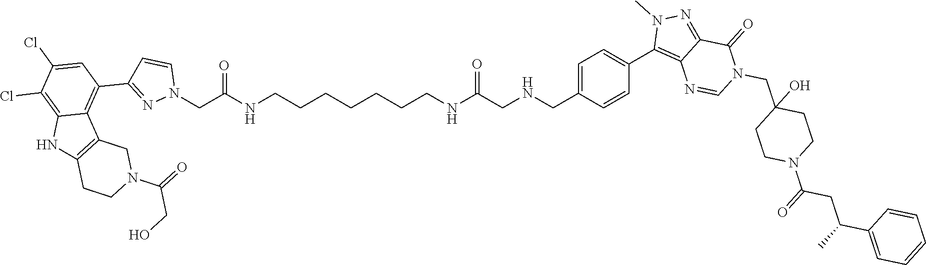 [SMILES: C[C@H](CC(=O)N1CCC(O)(Cn2cnc3c(-c4ccc(CNCC(=O)NCCCCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 47: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(7-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)heptyl)acetamide (XH168-147)

Example 47 was synthesized following the same procedure for preparing example 42 from linker 62 and intermediate 3. (white solid, 8.7 mg, yield 48%). ¹H NMR (400 MHz, Methanol-d₄) δ 7.98-7.88 (m, 1H), 7.75-7.58 (m, 5H), 7.32-7.15 (m, 6H), 6.55-6.51 (m, 1H), 4.92 (s, 2H), 4.65 (s, 1H), 4.42 (s, 1H), 4.31-4.27 (m, 2H), 4.21-4.13 (m, 2H), 4.08-4.01 (m, 4H), 3.97-3.80 (m, 4H), 3.70-3.59 (m, 2H), 3.34 (s, 1H), 3.22-3.19 (m, 6H), 3.04-2.80 (m, 3H), 2.77-2.69 (m, 1H), 2.61-2.45 (m, 1H), 1.63-1.50 (m, 5H), 1.38-1.32 (s, 12H).). MS (ESI) [M+H]⁻=1089.7.

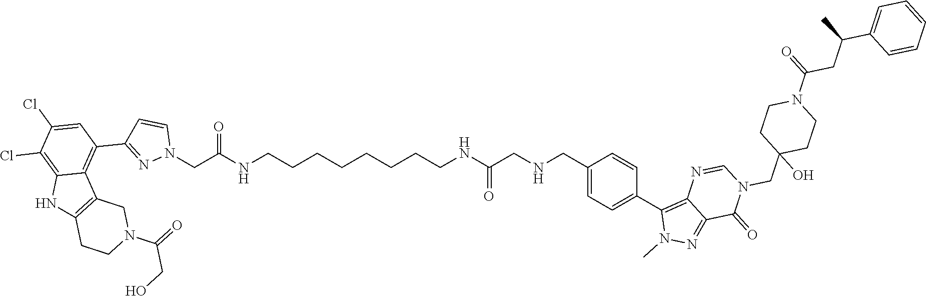 [SMILES: C[C@H](CC(=O)N1CCC(O)(Cn2cnc3c(-c4ccc(CNCC(=O)NCCCCCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 48: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(8-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)octyl)acetamide (XH168-148)

Example 48 was synthesized following the same procedure for preparing example 42 from linker 63 and intermediate 3. (white solid, 7.9 mg, yield 43%). ¹H NMR (400 MHz, Methanol-d₄) δ 7.98-7.88 (m, 1H), 7.75-7.61 (m, 5H), 7.32-7.15 (m, 6H), 6.55-6.53 (m, 1H), 4.92 (s, 2H), 4.64 (s, 1H), 4.37 (s, 1H), 4.32-4.29 (m, 2H), 4.21-4.02 (m, 6H), 3.95-3.80 (m, 4H), 3.72-3.59 (m, 2H), 3.34 (s, 1H), 3.25-3.21 (m, 6H), 3.04-2.85 (m, 3H), 2.80-2.68 (m, 1H), 2.61-2.44 (m, 1H), 1.63-1.47 (m, 5H), 1.37-1.26 (m, 14H). MS (ESI) [M+H]⁻=1103.6.

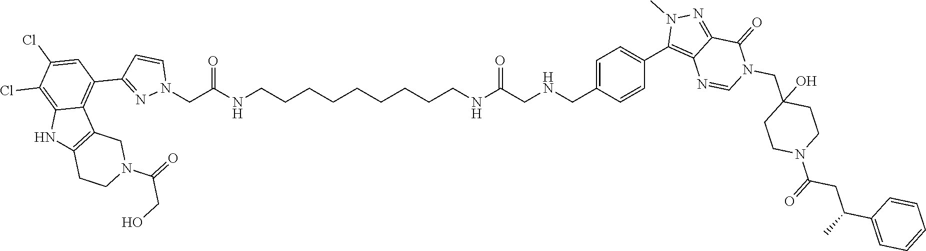 [SMILES: C[C@H](CC(=O)N1CCC(O)(Cn2cnc3c(-c4ccc(CNCC(=O)NCCCCCCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 49: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(9-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)nonyl)acetamide (XH168-149)

Example 49 was synthesized following the same procedure for preparing example 42 from linker 64 and intermediate 3. (white solid, 1.7 mg, yield 9%). ¹H NMR (400 MHz, Methanol-d₄) δ 8.00-7.90 (m, 1H), 7.73-7.64 (m, 5H), 7.30-7.17 (m, 6H), 6.56 (s, 1H), 4.92 (s, 2H), 4.38-4.31 (m, 3H), 4.22-4.06 (m, 6H), 3.99-3.85 (m, 3H), 3.79 (s, 2H), 3.69-3.60 (m, 2H), 3.23-3.13 (m, 6H), 3.06-2.98 (m, 2H), 2.89-2.85 (m, 2H), 2.81-2.69 (m, 1H), 2.61-2.45 (m, 1H), 1.61-1.49 (m, 5H), 1.38-1.28 (m, 16H).). MS (ESI) [M+H]⁺=1117.6.

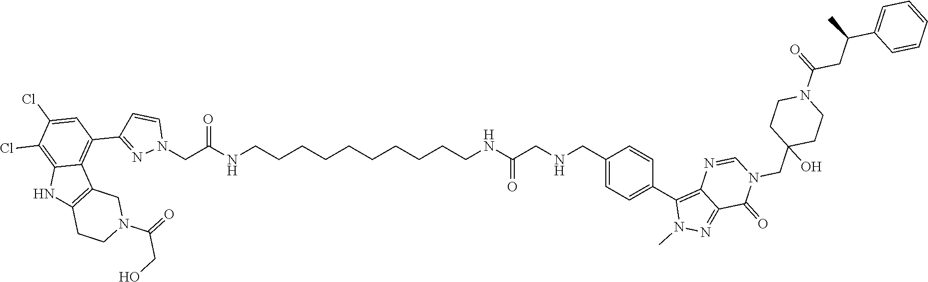 [SMILES: C[C@H](CC(=O)N1CCC(O)(Cn2cnc3c(-c4ccc(CNCC(=O)NCCCCCCCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 50: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(10-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)decyl)acetamide (XH168-150)

Example 50 was synthesized following the same procedure for preparing example 42 from linker 65 and intermediate 3. (white solid, 6.1 mg, yield 33%). ¹H NMR (400 MHz, Methanol-d₄) δ 7.99-7.92 (m, 1H), 7.75-7.64 (m, 5H), 7.32-7.14 (m, 6H), 6.55 (s, 1H), 4.92 (s, 2H), 4.64 (s, 1H), 4.41 (s, 1H), 4.32-4.30 (m, 2H), 4.21-4.02 (m, 6H), 3.96-3.79 (m, 4H), 3.70-3.59 (m, 2H), 3.24-3.20 (m, 6H), 3.04-2.86 (m, 3H), 2.80-2.68 (m, 1H), 2.61-2.45 (m, 1H), 1.64-1.46 (m, 5H), 1.42-1.22 (m, 18H). MS (ESI) [M+H]⁺=1131.7.

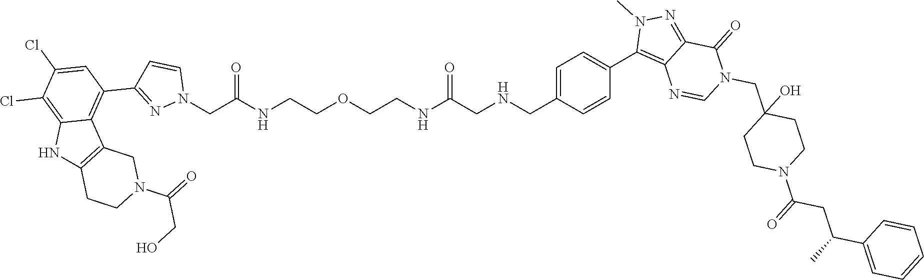 [SMILES: C[C@H](CC(=O)N1CCC(O)(Cn2cnc3c(-c4ccc(CNCC(=O)NCCOCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 51: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(2-(2-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)ethoxy)ethyl)acetamide (XH168-151)

Example 51 was synthesized following the same procedure for preparing example 42 from linker 66 and intermediate 3. (white solid, 4.8 mg, yield 27%). ¹H NMR (400 MHz, Methanol-d₄) δ 7.97-7.88 (m, 1H), 7.75 (s, 1H), 7.67-7.57 (m, 3H), 7.50-7.48 (m, 1H), 7.32-7.15 (m, 6H), 6.57-6.53 (m, 1H), 4.96 (s, 2H), 4.64 (s, 1H), 4.42 (s, 1H), 4.33-4.27 (m, 2H), 4.21-4.15 (m, 3H), 4.08-3.97 (m, 4H), 3.92-3.79 (m, 4H), 3.68-3.54 (m, 6H), 3.43-3.34 (m, 4H), 3.25-3.13 (m, 2H), 3.05-2.83 (m, 3H), 2.78-2.69 (m, 1H), 2.61-2.44 (m, 1H), 1.63-1.53 (m, 1H), 1.47-1.28 (m, 6H). MS (ESI) [M+H]⁺=1063.6.

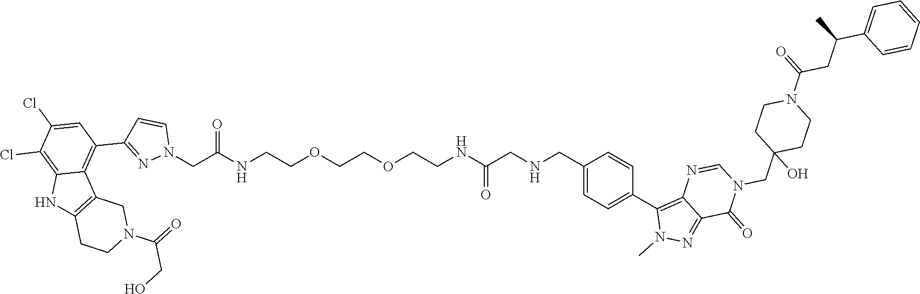 [SMILES: C[C@H](CC(=O)N1CCC(O)(Cn2cnc3c(-c4ccc(CNCC(=O)NCCOCCOCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 52: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11-dioxa-2,5-diazatridecan-13-yl)acetamide (XH168-152)

Example 52 was synthesized following the same procedure for preparing example 42 from linker 67 and intermediate 3. (white solid, 6.4 mg, yield 35%). ¹H NMR (400 MHz, Methanol-d₄) δ 7.96-7.84 (m, 1H), 7.76 (s, 1H), 7.60-7.50 (m, 4H), 7.30-7.15 (m, 6H), 6.59-6.50 (m, 1H), 4.96 (s, 2H), 4.64 (s, 1H), 4.45 (s, 1H), 4.33-4.28 (m, 2H), 4.21-4.15 (m, 3H), 4.09-3.97 (m, 4H), 3.93-3.76 (m, 4H), 3.68-3.54 (m, 10H), 3.42 (s, 4H), 3.25-3.13 (m, 2H), 3.04-2.83 (m, 3H), 2.77-2.69 (m, 1H), 2.61-2.44 (m, 1H), 1.58-1.56 (m, 1H), 1.47-1.25 (m, 6H). MS (ESI) [M+H]⁺=1107.6.

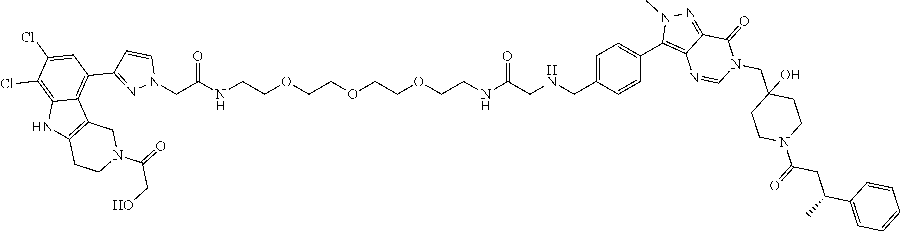 [SMILES: C[C@H](CC(=O)N1CCC(O)(Cn2cnc3c(-c4ccc(CNCC(=O)NCCOCCOCCOCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 53: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11,14-trioxa-2,5-diazahexadecan-16-yl)acetamide (XH168-153)

Example 53 was synthesized following the same procedure for preparing example 42 from linker 68 and intermediate 3. (white solid, 2.6 mg, yield 14%). ¹H NMR (400 MHz, Methanol-d₄) δ 7.97-7.89 (m, 1H), 7.76 (s, 1H), 7.67-7.53 (m, 4H), 7.30-7.17 (m 6H), 6.56-6.53 (m, 1H), 4.97 (s, 2H), 4.63 (s, 1H), 4.45 (s, 1H), 4.32-4.29 (m, 2H), 4.23-4.16 (m, 3H), 4.08-4.03 (m, 4H), 3.94-3.81 (m, 4H), 3.68-3.50 (m, 14H), 3.42 (s, 4H), 3.23-3.14 (m, 2H), 3.05-2.84 (m, 3H), 2.81-2.69 (m, 1H), 2.61-2.45 (m, 1H), 1.63-1.54 (m, 1H), 1.48-1.28 (m, 6H). MS (ESI) [M+H]⁺=1151.6.

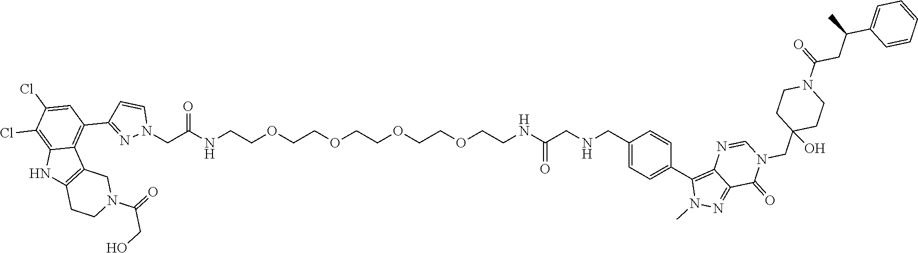 [SMILES: C[C@H](CC(=O)N1CCC(O)(Cn2cnc3c(-c4ccc(CNCC(=O)NCCOCCOCCOCCOCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 54: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11,14,17-tetraoxa-2,5-diazanonadecan-19-yl)acetamide (XH168-154)

Example 54 was synthesized following the same procedure for preparing example 42 from linker 69 and intermediate 3. (white solid, 3.5 mg, yield 35%). ¹H NMR (400 MHz, Methanol-d₄) δ 7.98-7.89 (m, 1H), 7.76 (s, 1H), 7.69-7.54 (m, 4H), 7.32-7.15 (m, 6H), 6.56-6.54 (m, 1H), 4.97 (s, 2H), 4.63 (s, 1H), 4.45 (s, 1H), 4.32-4.29 (m, 2H), 4.25-4.13 (m, 3H), 4.08-4.04 (m, 4H), 3.98-3.81 (m, 4H), 3.69-3.56 (m, 18H), 3.41 (s, 4H), 3.26-3.14 (m, 2H), 3.05-2.85 (m, 3H), 2.81-2.69 (m, 1H), 2.61-2.44 (m, 1H), 1.64-1.54 (m, 1H), 1.48-1.28 (m, 6H). MS (ESI) [M+H]⁺=1195.7.

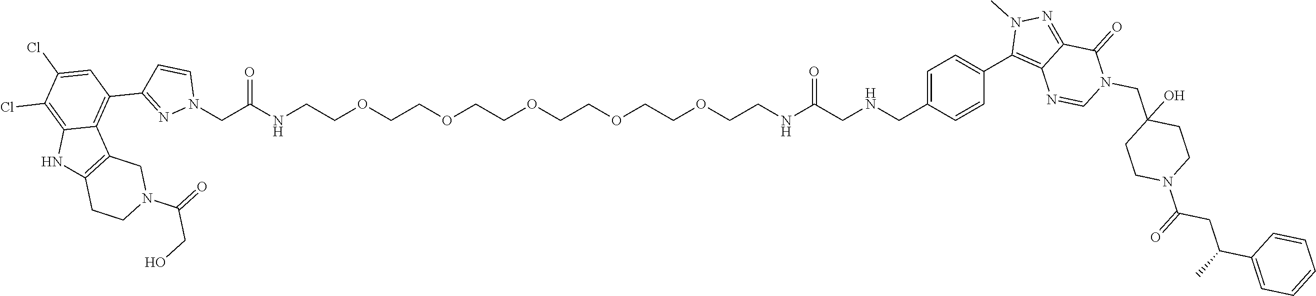 [SMILES: C[C@H](CC(=O)N1CCC(O)(Cn2cnc3c(-c4ccc(CNCC(=O)NCCOCCOCCOCCOCCOCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1]

### Example 55: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11,14,17,20-pentaoxa-2,5-diazadocosan-22-yl)acetamide (XH168-155)

Example 55 was synthesized following the same procedure for preparing example 42 from linker 70 and intermediate 3. (white solid, 0.9 mg, yield 4%). ¹H NMR (400 MHz, Methanol-d₄) δ 7.98-7.90 (m, 1H), 7.76 (s, 1H), 7.69-7.55 (m, 4H), 7.30-7.18 (m, 6H), 6.56-6.53 (m, 1H), 4.97 (m, 2H), 4.64 (s, 1H), 4.45 (s, 1H), 4.32-4.30 (m, 2H), 4.25-4.16 (m, 3H), 4.08-4.04 (m, 4H), 3.91-3.82 (m, 4H), 3.69-3.52 (m, 22H), 3.41 (s, 4H), 3.24-3.14 (m, 2H), 3.05-2.85 (m, 3H), 2.85-2.69 (m, 1H), 2.80-2.69 (m, 1H), 2.61-2.45 (m, 1H), 1.60-1.57 (m, 1H), 1.48-1.28 (m, 6H). MS (ESI) [M+H]⁺=1239.7.

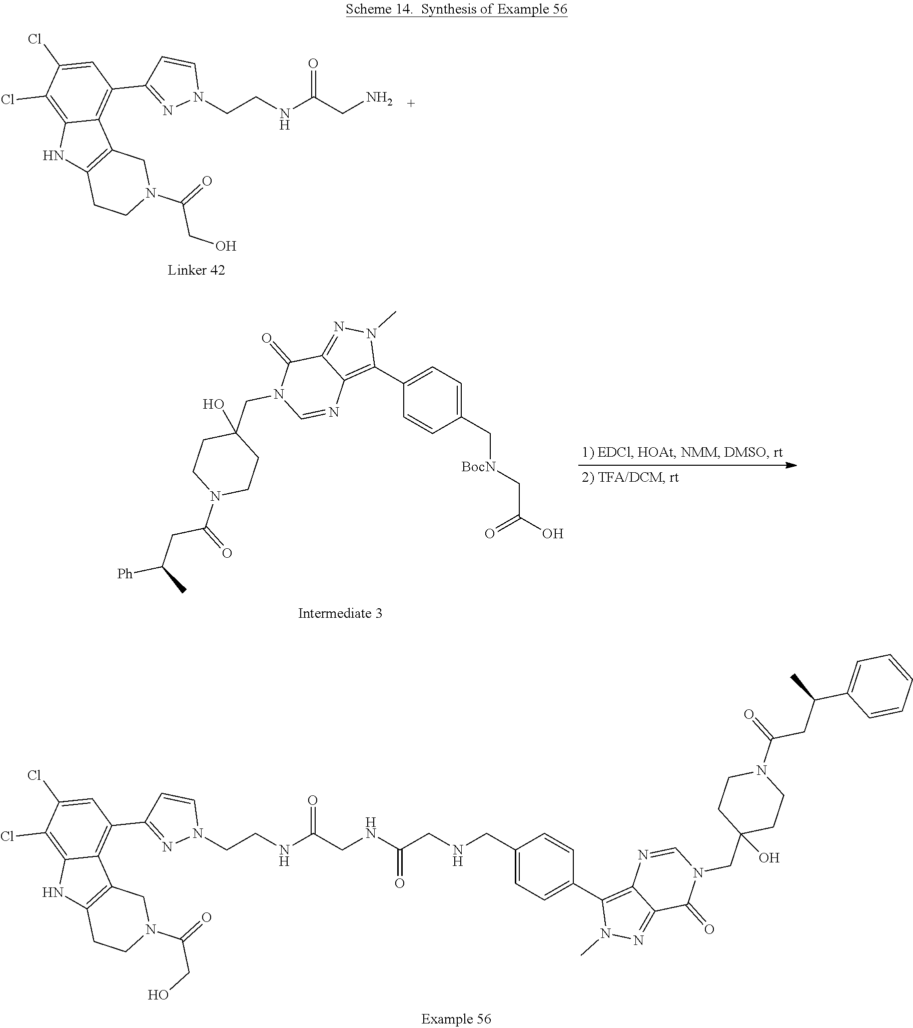 [SMILES: NCC(=O)NCCn1ccc(-c2cc(Cl)c(Cl)c3[nH]c4c(c23)CN(C(=O)CO)CC4)n1 ; C[C@H](CC(=O)N1CCC(O)(Cn2cnc3c(-c4ccc(CN(CC(=O)O)C(=O)OC(C)(C)C)cc4)n(C)nc3c2=O)CC1)c1ccccc1 ; C[C@H](CC(=O)N1CCC(O)(Cn2cnc3c(-c4ccc(CNCC(=O)NCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 56: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-2-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)acetamide (XH181-72)

To a solution of linker 42 (6.7 mg, 0.01 mmol) in DMF (0.5 mL) were added intermediate 3 (4.7 mg, 0.01 mmol, 1.0 equiv), 1-ethyl-3-(3-dimethylaminopropyl)carbodiimide (EDCI, 2.9 mg, 0.015 mmol, 1.5 equiv), 1-hydroxy-7-azabenzo-triazole (HOAt, 2.0 mg, 0.015 mmol, 1.5 equiv), and N-methylmorpholine (NMM, 3.0 mg, 0.03 mmol, 3.0 equiv). After being stirred for overnight at rt, the resulting mixture was purified by preparative HPLC to afford crude product. The crude product was dissolved in DCM/TFA (1:1, 1 mL). After being stirred at rt for 30 min, the resulting mixture was purified by preparative HPLC (10%-100% acetonitrile/0.1% TFA in H₂O) to afford titled compound as white solid (4.7 mg, yield 41%). ¹H NMR (400 MHz, Methanol-d₄) δ 8.01-7.93 (m 1H), 7.73-7.63 (m, 4H), 7.50 (d, J=8.0 Hz, 1H), 7.33-7.15 (m, 6H), 6.51-6.43 (m, 1H), 4.54 (s, 2H), 4.42-4.32 (m, 4H), 4.22-4.03 (m, 6H), 3.97-3.83 (m, 6H), 3.73-3.62 (m, 4H), 3.25-3.12 (m, 2H), 3.06-2.87 (m, 3H), 2.82-2.70 (m, 1H), 2.562-2.45 (m, 1H), 1.65-1.55 (m, 1H), 1.48-1.28 (m, 6H), 0.93-0.86 (m, 1H). MS (ESI) [M+H]⁺=1019.6.

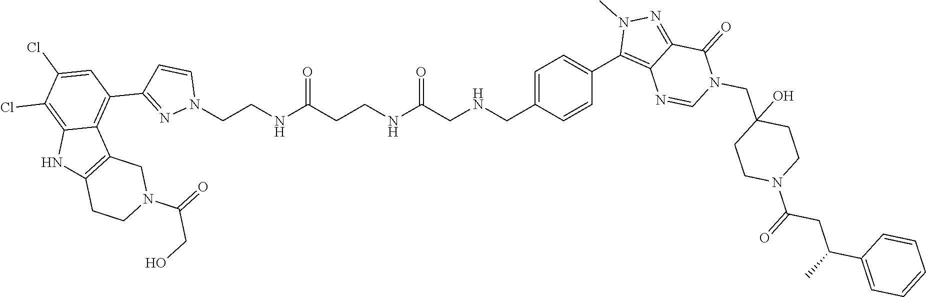 [SMILES: C[C@H](CC(=O)N1CCC(O)(Cn2cnc3c(-c4ccc(CNCC(=O)NCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 57: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)propenamide (XH181-73)

Example 57 was synthesized following the same procedure for preparing example 56 from linker 43 and intermediate 3. (white solid, 7.3 mg, yield 64%). ¹H NMR (400 MHz, Methanol-d₄) δ 7.93-7.87 (m, 1H), 7.69-7.53 (m, 4H), 7.54 (d, J=8.0 Hz, 1H), 7.28-7.12 (m, 6H), 6.46-6.37 (m, 1H), 4.49-4.47 (m, 2H), 4.35-4.26 (m, 4H), 4.18-4.14 (m, 2H), 4.07-4.03 (m, 4H), 3.99-3.86 (m, 2H), 3.79-3.56 (m, 6H), 3.47-3.42 (m, 2H), 3.23-3.10 (m, 2H), 3.01-2.83 (m, 3H), 2.77-2.65 (m, 1H), 2.57-2.35 (m, 3H), 1.60-1.49 (m, 1H), 1.44-1.23 (m, 6H), 0.87-0.81 (m, 1H). MS (ESI) [M+H]⁺=1033.6.

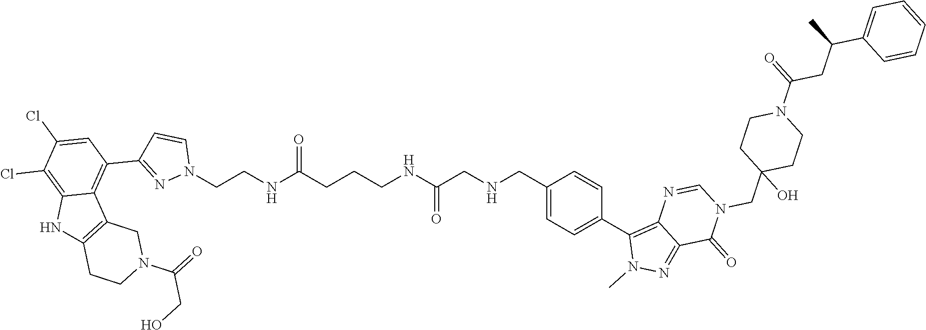 [SMILES: C[C@H](CC(=O)N1CCC(O)(Cn2cnc3c(-c4ccc(CNCC(=O)NCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 58: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-4-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)butanamide (XH181-90)

Example 58 was synthesized following the same procedure for preparing example 56 from linker 44 and intermediate 3. (white solid, 6.2 mg, yield 53%). ¹H NMR (400 MHz, Methanol-d₄) δ 7.98-7.93 (m, 1H), 7.73-7.59 (m, 5H), 7.30-7.15 (m, 6H), 6.50-6.42 (m, 1H), 4.53 (s, 2H), 4.37-4.31 (m, 4H), 4.26 (s, 1H), 4.20 (s, 1H), 4.12-4.06 (m, 4H), 4.00-3.82 (m, 2H), 3.77-3.60 (m, 6H), 3.23-3.14 (m, 4H), 3.05-2.88 (m, 3H), 2.81-2.69 (m, 1H), 2.61-2.45 (m, 1H), 2.24-2.19 (m, 2H), 1.80-1.77 (m, 2H), 1.64-1.28 (m, 7H), 0.96-0.85 (m, 1H). MS (ESI) [M+H]⁺=1047.6.

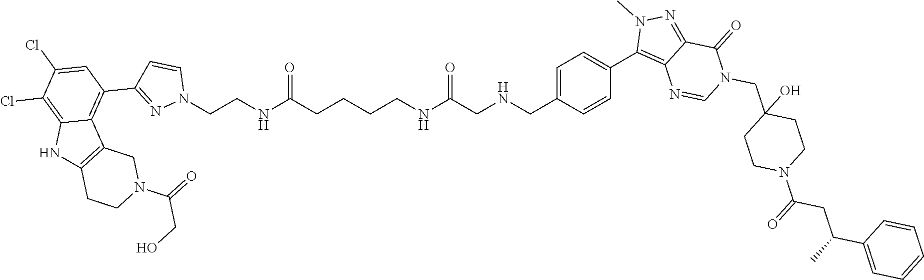 [SMILES: C[C@H](CC(=O)N1CCC(O)(Cn2cnc3c(-c4ccc(CNCC(=O)NCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 59: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-5-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)pentanamide (XH181-91)

Example 59 was synthesized following the same procedure for preparing example 56 from linker 45 and intermediate 3. (white solid, 7.2 mg, yield 61%). ¹H NMR (400 MHz, Methanol-d₄) δ 7.98-7.93 (m, 1H), 7.72-7.62 (m, 5H), 7.30-7.15 (m, 6H), 6.51-6.42 (m, 1H), 4.55 (s, 2H), 4.37-4.28 (m, 5H), 4.21-4.18 (m, 1H), 4.11-4.02 (m, 4H), 3.96-3.82 (m, 2H), 3.78-3.60 (m, 6H), 3.26-3.14 (m, 4H), 3.04-2.94 (m, 3H), 2.81-2.68 (m, 1H), 2.61-2.44 (m, 1H), 2.22-2.19 (m, 2H), 1.60-1.47 (m, 5H), 1.42-1.27 (m, 6H), 0.87 (t, J=12.0 Hz, 1H). MS (ESI) [M+H]⁺=1061.6.

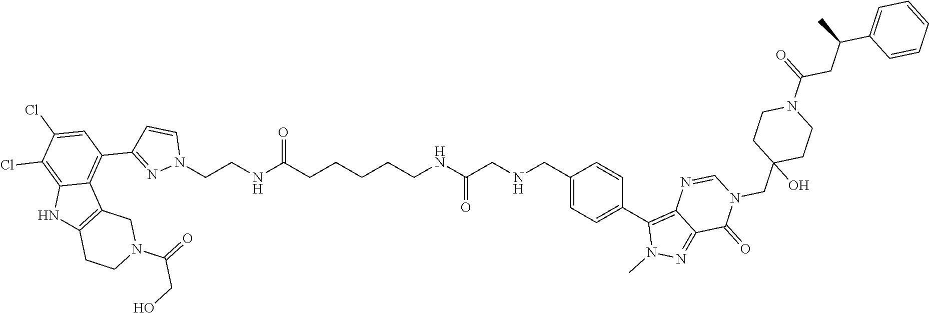 [SMILES: C[C@H](CC(=O)N1CCC(O)(Cn2cnc3c(-c4ccc(CNCC(=O)NCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 60: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-6-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)hexanamide (XH181-92)

Example 60 was synthesized following the same procedure for preparing example 56 from linker 46 and intermediate 3. (white solid, 5.8 mg, yield 49%). ¹H NMR (400 MHz, Methanol-d₄) δ 7.98-7.93 (m, 1H), 7.72-7.63 (m, 5H), 7.30-7.15 (m, 6H), 6.51-6.44 (m, 1H), 4.56-4.52 (m, 2H), 4.35-4.29 (m, 5H), 4.20 (s, 1H), 4.11-4.02 (m, 4H), 3.96-3.78 (m, 4H), 3.71-3.59 (m, 4H), 3.26-3.19 (m, 4H), 3.04-2.88 (m, 3H), 2.80-2.70 (m, 1H), 2.61-2.44 (m, 1H), 2.21-2.13 (m, 2H), 1.60-1.47 (m, 5H), 1.38-1.27 (m, 8H), 0.87 (t, J=12.0 Hz, 1H). MS (ESI) [M+H]⁺=1075.7.

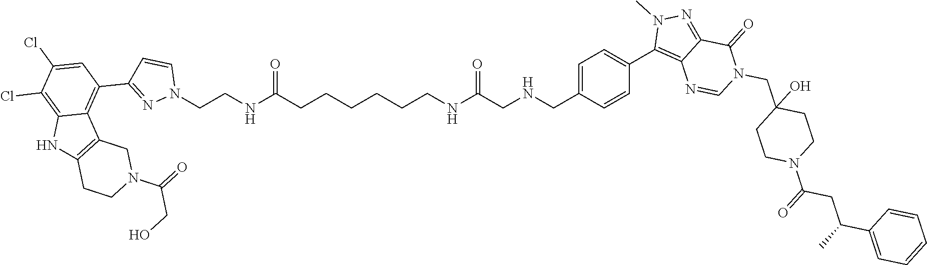 [SMILES: C[C@H](CC(=O)N1CCC(O)(Cn2cnc3c(-c4ccc(CNCC(=O)NCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 61: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-7-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)heptanamide (XH181-93)

Example 61 was synthesized following the same procedure for preparing example 56 from linker 47 and intermediate 3. (white solid, 7.2 mg, yield 60%). ¹H NMR (400 MHz, Methanol-d₄) δ 7.99-7.93 (m, 1H), 7.72-7.63 (m, 5H), 7.30-7.15 (m, 6H), 6.51-6.44 (m, 1H), 4.56-4.53 (m, 2H), 4.35-4.30 (m, 5H), 4.20 (s, 1H), 4.11-4.03 (m, 4H), 3.96-3.79 (m, 4H), 3.71-3.59 (m, 4H), 3.26-3.14 (m, 4H), 3.04-2.87 (m, 3H), 2.81-2.68 (m, 1H), 2.61-2.43 (m, 1H), 2.19-2.13 (m, 2H), 1.60-1.46 (m, 5H), 1.38-1.25 (m, 10H), 0.91-0.84 (m, 1H). MS (ESI) [M+H]⁺=1089.7.

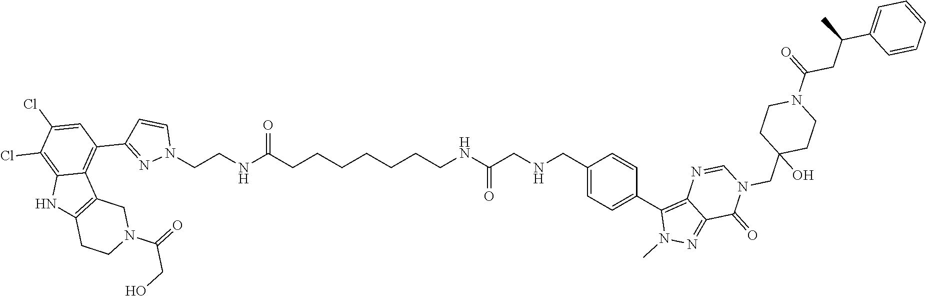 [SMILES: C[C@H](CC(=O)N1CCC(O)(Cn2cnc3c(-c4ccc(CNCC(=O)NCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 62: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-8-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)octanamide (XH181-94)

Example 62 was synthesized following the same procedure for preparing example 56 from linker 48 and intermediate 3. (white solid, 7.4 mg, yield 61%). ¹H NMR (400 MHz, Methanol-d₄) δ 7.98-7.93 (m, 1H), 7.71-7.61 (m, 5H), 7.30-7.16 (m, 6H), 6.51-6.44 (m, 1H), 4.57-4.54 (m, 2H), 4.35-4.32 (m, 5H), 4.20-4.03 (m, 5H), 3.96-3.79 (m, 4H), 3.72-3.59 (m, 4H), 3.26-3.17 (m, 4H), 3.04-2.88 (m, 3H), 2.80-2.68 (m, 1H), 2.61-2.44 (m, 1H), 2.18-2.10 (m, 2H), 1.60-1.44 (m, 5H), 1.38-1.23 (m, 12H), 0.87 (t, J=12.0 Hz, 1H). MS (ESI) [M+H]⁺=1103.7.

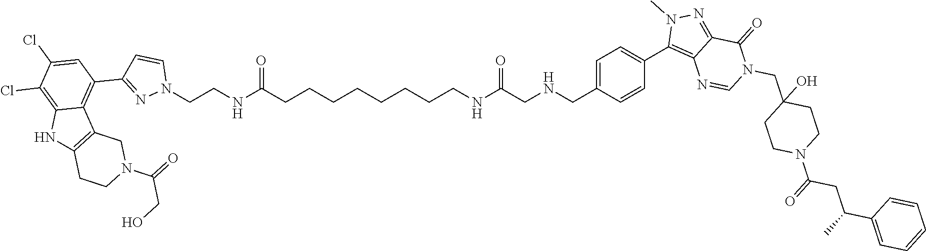 [SMILES: C[C@H](CC(=O)N1CCC(O)(Cn2cnc3c(-c4ccc(CNCC(=O)NCCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 63: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-9-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)nonanamide (XH181-95)

Example 63 was synthesized following the same procedure for preparing example 56 from linker 49 and intermediate 3. (white solid, 7.2 mg, yield 58%). ¹H NMR (400 MHz, Methanol-d₄) δ 7.99-7.94 (m, 1H), 7.72-7.62 (m, 5H), 7.30-7.17 (m, 6H), 6.51-6.44 (m, 1H), 4.58-4.54 (m, 2H), 4.37-4.31 (m, 5H), 4.21-4.03 (m, 5H), 3.97-3.80 (m, 4H), 3.72-3.60 (m, 4H), 3.26-3.14 (m, 4H), 3.05-2.88 (m, 3H), 2.81-2.68 (m, 1H), 2.61-2.44 (m, 1H), 2.18-2.12 (m, 2H), 1.64-1.42 (m, 5H), 1.38-1.20 (m, 14H), 0.88 (t, J=12.0 Hz, 1H). MS (ESI) [M+H]⁺=1117.9.

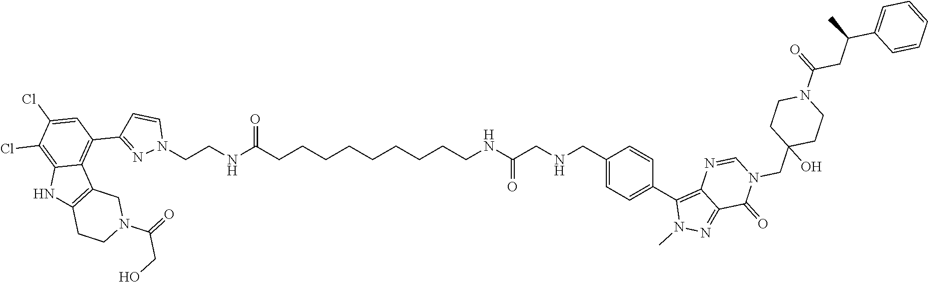 [SMILES: C[C@H](CC(=O)N1CCC(O)(Cn2cnc3c(-c4ccc(CNCC(=O)NCCCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 64: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-10-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)decanamide (XH181-96)

Example 64 was synthesized following the same procedure for preparing example 56 from linker 50 and intermediate 3. (white solid, 6.8 mg, yield 55%). ¹H NMR (400 MHz, Methanol-d₄) δ 8.00-7.94 (m, 1H), 7.73-7.62 (m, 5H), 7.31-7.017 (m, 6H), 6.51-6.44 (m, 1H), 4.59-4.55 (m, 2H), 4.36-4.31 (m, 5H), 4.21-4.03 (m, 5H), 3.97-3.80 (m, 4H), 3.72-3.60 (m, 4H), 3.26-3.14 (m, 4H), 3.05-2.88 (m, 3H), 2.81-2.68 (m, 1H), 2.61-2.44 (m, 1H), 2.18-2.12 (m, 2H), 1.64-1.47 (m, 5H), 1.38-1.18 (m, 16H), 0.88 (t, J=12.0 Hz, 1H). MS (ESI) [M+H]⁺=1131.7.

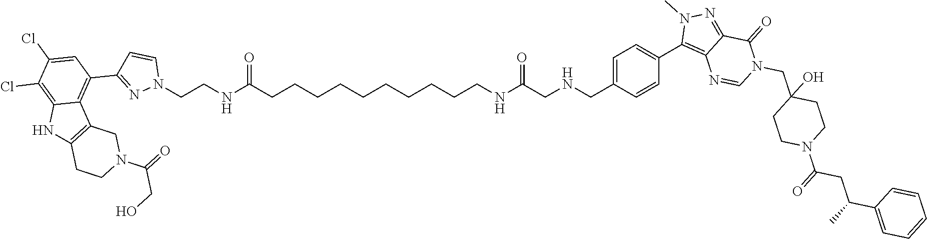 [SMILES: C[C@H](CC(=O)N1CCC(O)(Cn2cnc3c(-c4ccc(CNCC(=O)NCCCCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 65: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-11-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)undecanamide (XH181-97)

Example 65 was synthesized following the same procedure for preparing example 56 from linker 51 and intermediate 3. (white solid, 5.6 mg, yield 44%). ¹H NMR (400 MHz, Methanol-d₄) δ 8.00-7.95 (m, 1H), 7.75-7.62 (m, 5H), 7.31-7.17 (m, 6H), 6.50-6.44 (m, 1H), 4.59-4.55 (m, 2H), 4.37-4.32 (m, 5H), 4.21-4.03 (m, 5H), 3.98-3.80 (m, 4H), 3.74-3.50 (m, 4H), 3.27-3.21 (m, 4H), 3.05-2.88 (m, 3H), 2.81-2.69 (m, 1H), 2.61-2.44 (m, 1H), 2.18-2.10 (m, 2H), 1.64-1.49 (m, 5H), 1.38-1.19 (m, 18H), 0.91-0.84 (m, 1H). MS (ESI) [M+H]⁺=1145.8.

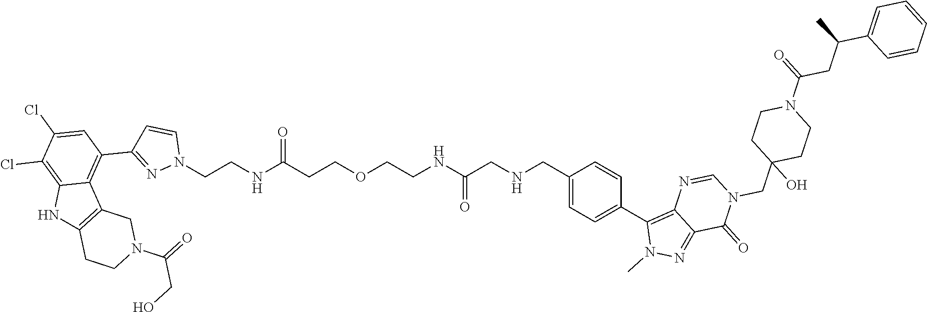 [SMILES: C[C@H](CC(=O)N1CCC(O)(Cn2cnc3c(-c4ccc(CNCC(=O)NCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 66: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(2-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)ethoxy)propenamide (XH181-99)

Example 66 was synthesized following the same procedure for preparing example 56 from linker 52 and intermediate 3. (white solid, 8.4 mg, yield 70%). ¹H NMR (400 MHz, Methanol-d₄) δ 7.96-7.91 (m, 1H), 7.69-7.57 (m, 5H), 7.30-7.15 (m, 6H), 6.50-6.43 (m, 1H), 4.53 (s, 2H), 4.35-4.24 (m, 5H), 4.18 (s, 1H), 4.11-3.98 (m, 4H), 3.93-3.89 (m, 1H), 3.82-3.78 (m, 2H), 3.69-3.58 (m, 6H), 3.52-3.48 (m, 2H), 3.37 (m, 2H), 3.25-3.13 (m, 2H), 3.04-2.85 (m, 3H), 2.80-2.68 (m, 1H), 2.61-2.40 (m, 3H), 1.62-1.53 (m, 1H), 1.46-1.25 (m, 6H), 0.86 (t, J=12.0 Hz, 1H). MS (ESI) [M+H]⁺=1077.6.

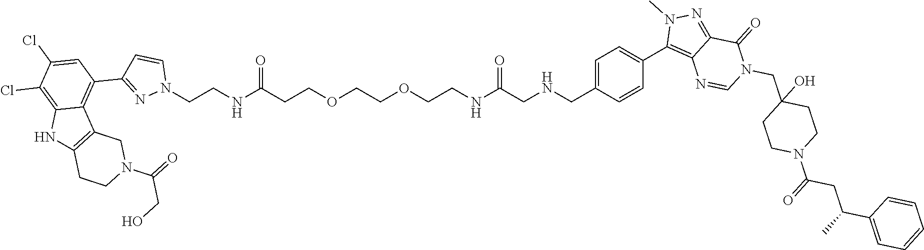 [SMILES: C[C@H](CC(=O)N1CCC(O)(Cn2cnc3c(-c4ccc(CNCC(=O)NCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 67: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(2-(2-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)ethoxy)ethoxy)propenamide (XH181-100)

Example 67 was synthesized following the same procedure for preparing example 56 from linker 53 and intermediate 3. (white solid, 8.2 mg, yield 66%). ¹H NMR (400 MHz, Methanol-d₄) δ 7.96-7.91 (m, 1H), 7.70-7.57 (m, 5H), 7.30-7.15 (m, 6H), 6.50-6.43 (m, 1H), 4.54-4.53 (m, 2H), 4.34-4.26 (m, 5H), 4.18 (s, 1H), 4.10-3.98 (m, 4H), 3.90-3.89 (m, 1H), 3.82 (s, 2H), 3.70-3.65 (m, 6H), 3.56-3.52 (m, 6H), 3.41-3.39 (tm, 2H), 3.25-3.13 (m, 2H), 3.04-2.87 (m, 3H), 2.80-2.68 (m, 1H), 2.61-2.39 (m, 3H), 1.63-1.53 (m, 1H), 1.47-1.26 (m, 6H), 0.86 (t, J=12.0 Hz, 1H). MS (ESI) [M+H]⁺=1121.7.

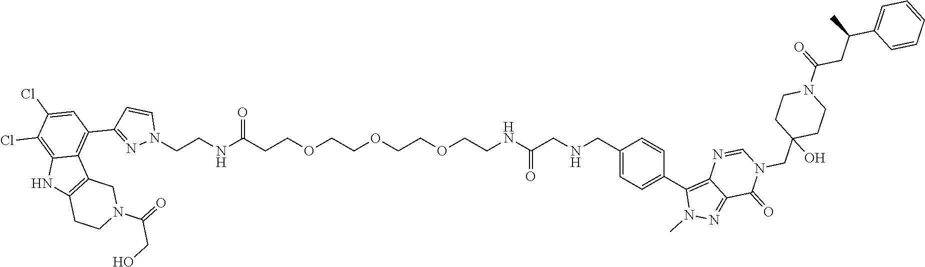 [SMILES: C[C@H](CC(=O)N1CCC(O)(Cn2cnc3c(-c4ccc(CNCC(=O)NCCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 68: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-((1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11-dioxa-2,5-diazatridecan-13-yl)oxy)propenamide (XH181-101)

Example 68 was synthesized following the same procedure for preparing example 56 from linker 54 and intermediate 3. (white solid, 9.0 mg, yield 70%). ¹H NMR (400 MHz, Methanol-d₄) δ 7.97-7.92 (m, 1H), 7.71-7.62 (m, 5H), 7.31-7.17 (m, 6H), 6.50-6.43 (m, 1H), 4.56-4.54 (m, 2H), 4.35-4.28 (m, 5H), 4.20 (s, 1H), 4.09-3.99 (m, 4H), 3.90 (s, 1H), 3.83-3.80 (m 2H), 3.67 (s, 6H), 3.56-3.51 (m, 10H), 3.40 (s, 2H), 3.25-3.13 (m, 2H), 3.04-2.85 (m, 3H), 2.80-2.68 (m, 1H), 2.61-2.40 (m, 3H), 1.63-1.53 (m, 1H), 1.47-1.27 (m, 6H), 0.87 (t, J=12.0 Hz, 1H). MS (ESI) [M+H]⁺=1165.7.

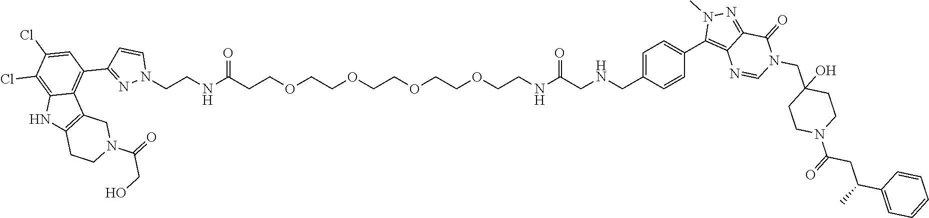 [SMILES: C[C@H](CC(=O)N1CCC(O)(Cn2cnc3c(-c4ccc(CNCC(=O)NCCOCCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 69: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-1-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)-3,6,9,12-tetraoxapentadecan-15-amide (XH181-102)

Example 69 was synthesized following the same procedure for preparing example 56 from linker 55 and intermediate 3. (white solid, 8.1 mg, yield 61%). ¹H NMR (400 MHz, Methanol-d₄) δ 7.97-7.92 (m, 1H), 7.71-7.63 (m, 5H), 7.31-7.16 (m, 6H), 6.50-6.44 (m, 1H), 4.57-4.54 (m, 2H), 4.34-4.29 (m, 5H), 4.20 (s, 1H), 4.09-4.01 (m, 4H), 3.95-3.89 (m, 1H), 3.82 (s, 2H), 3.67 (s, 6H), 3.55 (s, 14H), 3.41-3.39 (m, 2H), 3.25-3.13 (m, 2H), 3.04-2.85 (m, 3H), 2.80-2.68 (m, 1H), 2.61-2.39 (m, 3H), 1.63-1.53 (m, 1H), 1.47-1.27 (m, 6H), 0.87 (t, J=12.0 Hz, 1H). MS (ESI) [M+H]⁺=1209.7.

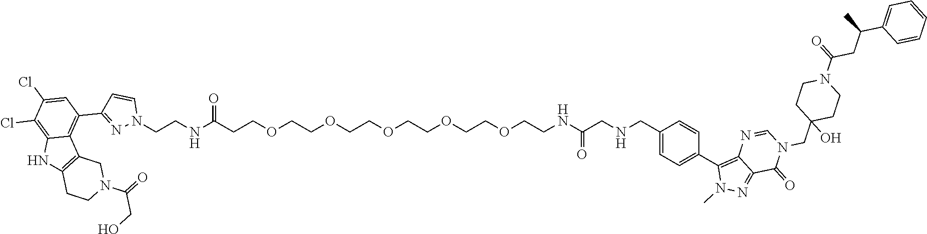 [SMILES: C[C@H](CC(=O)N1CCC(O)(Cn2cnc3c(-c4ccc(CNCC(=O)NCCOCCOCCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 70: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-1-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)-3,6,9,12,15-pentaoxaoctadecan-18-amide (XH181-103)

Example 70 was synthesized following the same procedure for preparing example 56 from linker 66 and intermediate 3. (white solid, 7.4 mg, yield 54%). ¹H NMR (400 MHz, Methanol-d₄) δ 7.97-7.92 (m, 1H), 7.72-7.65 (m, 5H), 7.30-7.16 (m, 6H), 6.51-6.44 (m, 1H), 4.57-4.54 (m, 2H), 4.34-4.30 (m, 5H), 4.20 (s, 1H), 4.10-4.01 (m, 4H), 3.94-3.91 (m, 1H), 3.82 (s, 2H), 3.88-3.86 (m, 6H), 3.56-3.53 (m, 18H), 3.41-3.40 (m, 2H), 3.26-3.13 (m, 2H), 3.05-2.87 (m, 3H), 2.80-2.68 (m, 1H), 2.61-2.39 (m, 3H), 1.63-1.53 (m, 1H), 1.47-1.27 (m, 6H), 0.87 (t, J=12.0 Hz, 1H). MS (ESI) [M+H]⁺=1253.8.

Examples 71-96 were synthesized following the same procedure for preparing example 56.

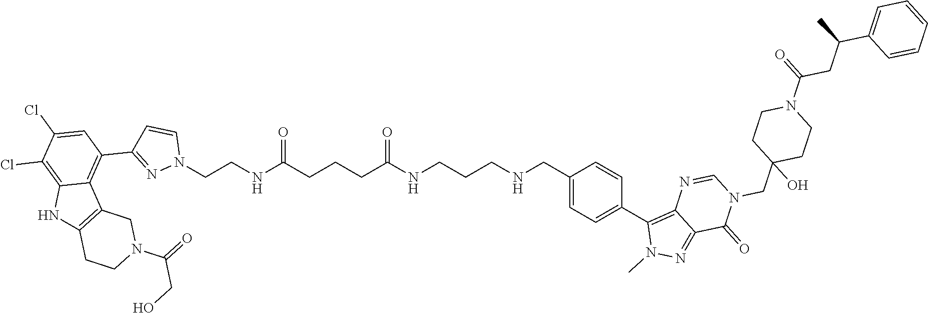 [SMILES: C[C@H](CC(=O)N1CCC(O)(Cn2cnc3c(-c4ccc(CNCCCNC(=O)CCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 71: (R)—N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N5-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl) glutaramide (Xh188-48)

¹H NMR (400 MHz, Methanol-d₄) δ 8.00-7.89 (m, 1H), 7.81-7.58 (m, 5H), 7.36-7.11 (m, 6H), 6.59-6.38 (m, 1H), 4.58-4.49 (m, 2H), 4.44-4.33 (m, 3H), 4.32-4.14 (m, 3H), 4.15-4.00 (m, 4H), 3.97-3.80 (m, 2H), 3.73-3.59 (m, 5H), 3.29-2.98 (m, 4H), 2.98-2.80 (m, 3H), 2.83-2.68 (m, 1H), 2.66-2.47 (m, 1H), 2.49-2.35 (m, 1H), 2.37-2.11 (m, 3H), 1.96-1.84 (m, 3H), 1.70-1.51 (m, 1H), 1.51-1.21 (m, 7H). MS (ESI) m/z 1075.4 [M+H]⁺.

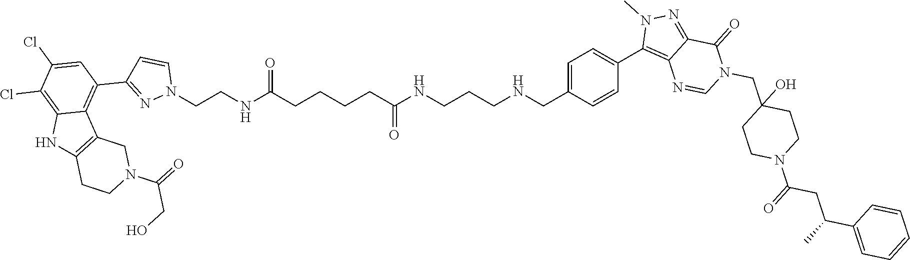 [SMILES: C[C@H](CC(=O)N1CCC(O)(Cn2cnc3c(-c4ccc(CNCCCNC(=O)CCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 72: (R)—N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N6-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl) adipamide (Xh188-49)

¹H NMR (400 MHz, Methanol-d₄) δ 8.01-7.85 (m, 1H), 7.74-7.58 (m, 5H), 7.39-7.09 (m, 6H), 6.55-6.34 (m, 1H), 4.53 (s, 2H), 4.39-4.30 (m, 3H), 4.28-4.22 (m, 2H), 4.21-4.16 (m, 1H), 4.14-4.02 (m, 4H), 4.01-3.76 (m, 2H), 3.74-3.52 (m, 5H), 3.26-3.03 (m, 4H), 3.02-2.83 (m, 3H), 2.81-2.66 (m, 1H), 2.64-2.43 (m, 1H), 2.26-2.15 (m, 4H), 1.95-1.85 (m, 2H), 1.67-1.52 (m, 5H), 1.56-1.14 (m, 8H). MS (ESI) m/z 1091.5 [M+H]⁺.

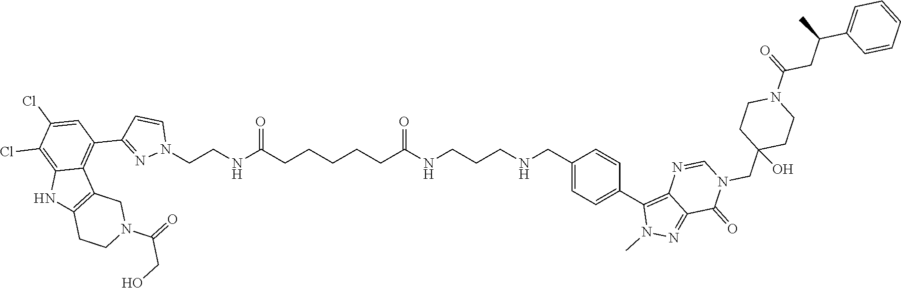 [SMILES: C[C@H](CC(=O)N1CCC(O)(Cn2cnc3c(-c4ccc(CNCCCNC(=O)CCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 73: (R)—N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N7-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)heptanediamide (Xh188-50)

¹H NMR (400 MHz, Methanol-d₄) δ 8.03-7.81 (m, 1H), 7.79-7.57 (m, 5H), 7.35-7.09 (m, 6H), 6.55-6.29 (m, 1H), 4.60-4.45 (m, 2H), 4.38-4.29 (m, 3H), 4.29-4.22 (m, 2H), 4.21-4.16 (m, 1H), 4.15-4.03 (m, 4H), 4.02-3.79 (m, 2H), 3.76-3.52 (m, 4H), 3.26-3.11 (m, 2H), 3.11-3.02 (m, 2H), 3.02-2.83 (m, 3H), 2.83-2.65 (m, 1H), 2.62-2.41 (m, 1H), 2.22-2.12 (m, 4H), 1.97-1.84 (m, 2H), 1.66-1.51 (m, 5H), 1.50-1.17 (m, 10H). MS (ESI) m/z 1103.5 [M+H]⁺.

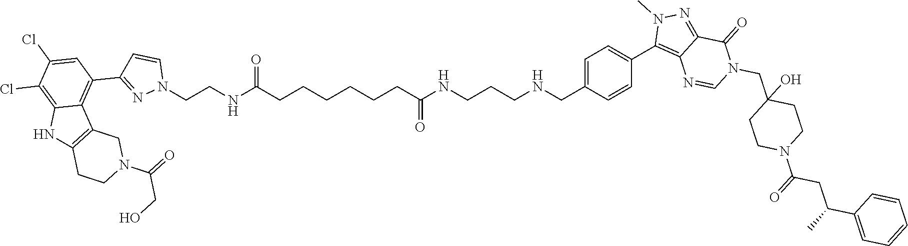 [SMILES: C[C@H](CC(=O)N1CCC(O)(Cn2cnc3c(-c4ccc(CNCCCNC(=O)CCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 74: (R)—N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N8-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)octanediamide (Xh188-51)

¹H NMR (400 MHz, Methanol-d₄) δ 8.03-7.84 (m, 1H), 7.78-7.62 (m, 5H), 7.32-7.15 (m, 6H), 6.53-6.40 (m, 1H), 4.62-4.50 (m, 2H), 4.37-4.30 (m, 3H), 4.28-4.22 (m, 2H), 4.22-4.15 (m, 1H), 4.15-4.06 (m, 4H), 4.03-3.80 (m, 2H), 3.78-3.54 (m, 5H), 3.26-3.00 (m, 4H), 3.00-2.84 (m, 3H), 2.83-2.66 (m, 1H), 2.65-2.41 (m, 1H), 2.23-2.12 (m, 4H), 1.96-1.85 (m, 2H), 1.63-1.51 (m, 5H), 1.48-1.25 (m, 12H). MS (ESI) m/z 1117.5 [M+H]⁺.

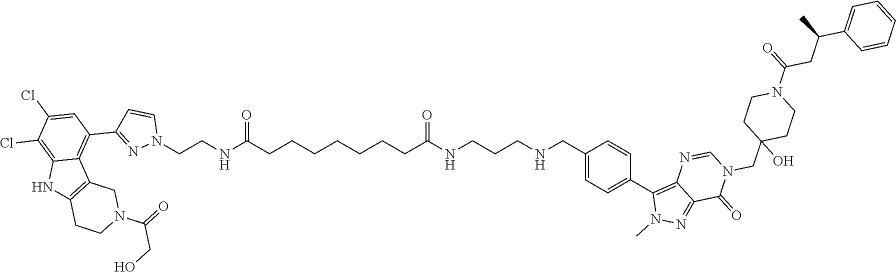 [SMILES: C[C@H](CC(=O)N1CCC(O)(Cn2cnc3c(-c4ccc(CNCCCNC(=O)CCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 75: (R)—N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N9-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)nonanediamide (Xh188-52)

¹H NMR (400 MHz, Methanol-d₄) δ 8.01-7.86 (m, 1H), 7.76-7.59 (m, 5H), 7.33-7.12 (m, 6H), 6.54-6.41 (m, 1H), 4.60-4.49 (m, 2H), 4.38-4.29 (m, 3H), 4.29-4.23 (m, 2H), 4.23-4.15 (m, 1H), 4.15-4.03 (m, 4H), 4.02-3.79 (m, 2H), 3.77-3.55 (m, 4H), 3.27-3.15 (m, 2H), 3.14-3.03 (m, 2H), 3.03-2.85 (m, 3H), 2.85-2.68 (m, 1H), 2.64-2.40 (m, 1H), 2.23-2.08 (m, 4H), 1.97-1.83 (m, 2H), 1.61-1.47 (m, 5H), 1.50-1.13 (m, 14H). MS (ESI) m/z 1132.6 [M+H]⁺.

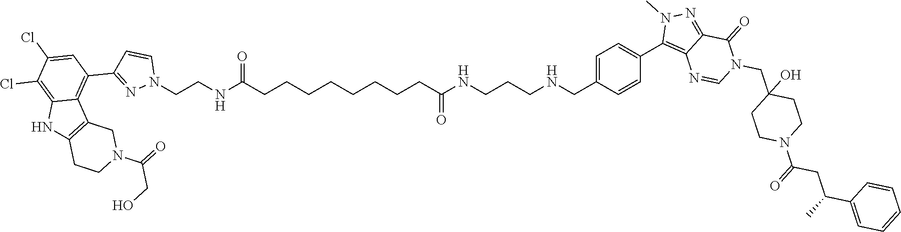 [SMILES: C[C@H](CC(=O)N1CCC(O)(Cn2cnc3c(-c4ccc(CNCCCNC(=O)CCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 76: (R)—N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N10-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)decanediamide (Xh188-53)

¹H NMR (400 MHz, Methanol-d₄) δ 8.05-7.83 (m, 1H), 7.83-7.58 (m, 5H), 7.33-7.08 (m, 6H), 6.55-6.36 (m, 1H), 4.64-4.47 (m, 2H), 4.43-4.30 (m, 3H), 4.30-4.25 (m, 2H), 4.24-4.15 (m, 1H), 4.15-4.02 (m, 4H), 4.00-3.79 (m, 2H), 3.77-3.51 (m, 5H), 3.28-3.14 (m, 2H), 3.14-3.03 (m, 2H), 3.02-2.84 (m, 3H), 2.84-2.66 (m, 1H), 2.65-2.38 (m, 1H), 2.23-2.07 (m, 4H), 1.97-1.84 (m, 2H), 1.64-1.45 (m, 5H), 1.45-1.11 (m, 16H). MS (ESI) m/z 1145.6 [M+H]⁺.

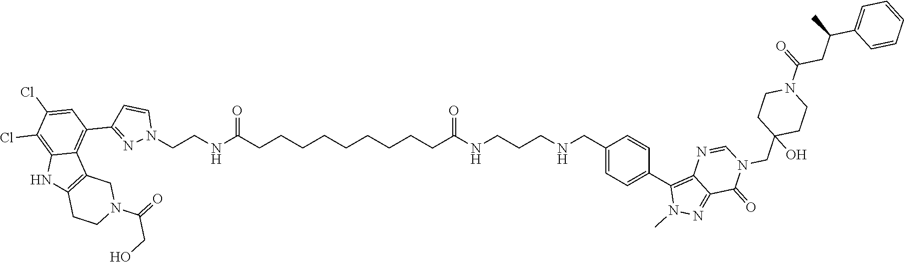 [SMILES: C[C@H](CC(=O)N1CCC(O)(Cn2cnc3c(-c4ccc(CNCCCNC(=O)CCCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 77: (R)—N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N11-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)undecanediamide (Xh188-54)

¹H NMR (400 MHz, Methanol-d₄) δ 8.03-7.87 (m, 1H), 7.81-7.58 (m, 5H), 7.37-7.10 (m, 6H), 6.56-6.39 (m, 1H), 4.63-4.51 (m, 2H), 4.41-4.30 (m, 3H), 4.30-4.25 (m, 2H), 4.24-4.15 (m, 1H), 4.14-4.07 (m, 4H), 4.05-3.81 (m, 2H), 3.78-3.46 (m, 4H), 3.28-3.16 (m, 2H), 3.15-3.03 (m, 2H), 3.03-2.83 (m, 3H), 2.81-2.65 (m, 1H), 2.64-2.41 (m, 1H), 2.25-2.06 (m, 4H), 1.99-1.83 (m, 2H), 1.62-1.46 (m, 5H), 1.47-1.08 (m, 18H). MS (ESI) m/z 1159.6 [M+H]⁺.

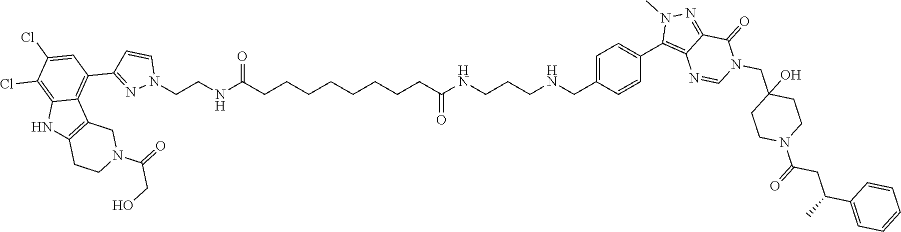 [SMILES: C[C@H](CC(=O)N1CCC(O)(Cn2cnc3c(-c4ccc(CNCCCNC(=O)CCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 78: (R)—N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N12-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl) dodecanediamide (Xh188-55)

¹H NMR (400 MHz, Methanol-d₄) δ 8.03-7.90 (m, 1H), 7.84-7.57 (m, 5H), 7.35-7.08 (m, 6H), 6.57-6.37 (m, 1H), 4.64-4.51 (m, 2H), 4.44-4.30 (m, 3H), 4.30-4.25 (m, 2H), 4.25-4.16 (m, 1H), 4.16-4.02 (m, 4H), 4.02-3.79 (m, 2H), 3.78-3.56 (m, 5H), 3.27-3.15 (m, 2H), 3.15-3.03 (m, 2H), 3.04-2.80 (m, 3H), 2.83-2.65 (m, 1H), 2.64-2.39 (m, 1H), 2.27-2.10 (m, 4H), 1.96-1.83 (m, 2H), 1.63-1.49 (m, 5H), 1.33-1.15 (m, 20H). MS (ESI) m/z 1173.6 [M+H]⁺.

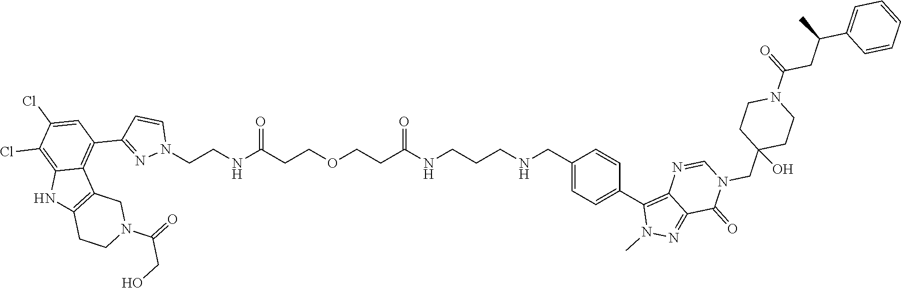 [SMILES: C[C@H](CC(=O)N1CCC(O)(Cn2cnc3c(-c4ccc(CNCCCNC(=O)CCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 79: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(3-((3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)amino)-3-oxopropoxy)propenamide (Xh188-56)

¹H NMR (400 MHz, Methanol-d₄) δ 8.03-7.82 (m, 1H), 7.76-7.51 (m, 5H), 7.36-7.09 (m, 6H), 6.56-6.34 (m, 1H), 4.59-4.44 (m, 2H), 4.36-4.29 (m, 3H), 4.27-4.14 (m, 3H), 4.14-4.02 (m, 4H), 3.98-3.79 (m, 2H), 3.76-3.58 (m, 9H), 3.28-3.11 (m, 2H), 3.11-2.99 (m, 2H), 2.95-2.86 (m, 2H), 2.83-2.74 (m, 1H), 2.74-2.53 (m, 1H), 2.49-2.39 (m, 4H), 1.98-1.80 (m, 2H), 1.71-1.51 (m, 1H), 1.50-1.19 (m, 7H). MS (ESI) m/z 1105.3 [M+H]⁺.

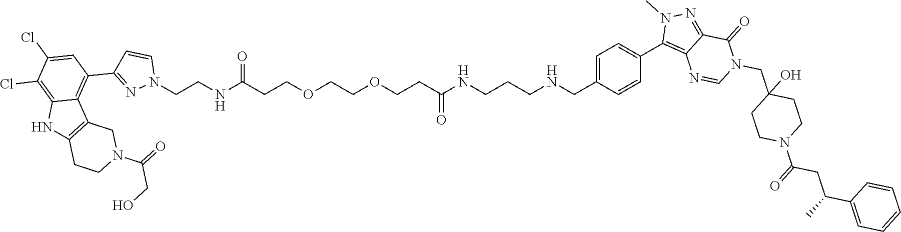 [SMILES: C[C@H](CC(=O)N1CCC(O)(Cn2cnc3c(-c4ccc(CNCCCNC(=O)CCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 80: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(2-(3-((3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)amino)-3-oxopropoxy)ethoxy)propenamide (Xh188-57)

¹H NMR (400 MHz, Methanol-d₄) δ 8.01-7.84 (m, 1H), 7.72-7.61 (m, 5H), 7.36-7.12 (m, 6H), 6.54-6.38 (m, 1H), 4.62-4.47 (m, 2H), 4.38-4.30 (m, 3H), 4.25-4.15 (m, 3H), 4.11-3.99 (m, 4H), 3.97-3.80 (m, 2H), 3.76-3.60 (m, 9H), 3.57-3.51 (m, 4H), 3.28-3.15 (m, 2H), 3.12-3.02 (m, 2H), 2.97-2.90 (m, 2H), 2.89-2.76 (m, 1H), 2.75-2.54 (m, 1H), 2.47-2.37 (m, 4H), 1.96-1.80 (m, 2H), 1.70-1.52 (m, 1H), 1.50-1.24 (m, 7H). MS (ESI) m/z 1149.3 [M+H]⁺.

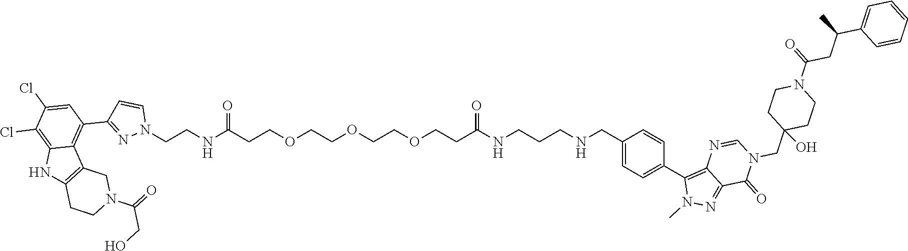 [SMILES: C[C@H](CC(=O)N1CCC(O)(Cn2cnc3c(-c4ccc(CNCCCNC(=O)CCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 81: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-((1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-7-oxo-10,13-dioxa-2,6-diazapentadecan-15-yl)oxy)propenamide (Xh188-58)

¹H NMR (400 MHz, Methanol-d₄) δ 8.00-7.85 (m, 1H), 7.75-7.58 (m, 5H), 7.36-7.11 (m, 6H), 6.54-6.39 (m, 1H), 4.64-4.47 (m, 2H), 4.35-4.26 (m, 3H), 4.27-4.16 (m, 3H), 4.11-3.97 (m, 4H), 3.96-3.78 (m, 2H), 3.73-3.61 (m, 9H), 3.57-3.51 (m, 8H), 3.27-3.15 (m, 2H), 3.15-3.07 (m, 2H), 2.95-2.75 (m, 3H), 2.75-2.53 (m, 1H), 2.47-2.38 (m, 4H), 1.96-1.81 (m, 2H), 1.68-1.50 (m, 1H), 1.50-1.21 (m, 7H). MS (ESI) m/z 1193.6 [M+H]⁺.

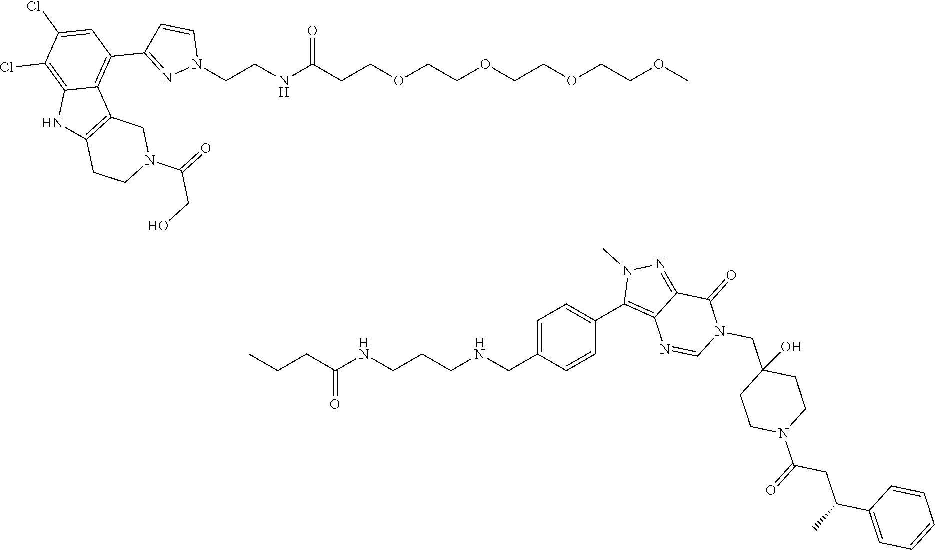

### Example 82: (R)—N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N16-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)-4,7,10,13-tetraoxahexadecanediamide (Xh188-59)

¹H NMR (400 MHz, Methanol-d₄) δ 8.03-7.85 (m, 1H), 7.78-7.60 (m, 5H), 7.36-7.12 (m, 6H), 6.56-6.39 (m, 1H), 4.63-4.50 (m, 2H), 4.35-4.30 (m, 3H), 4.26-4.17 (m, 3H), 4.13-4.05 (m, 4H), 4.01-3.88 (m, 2H), 3.75-3.64 (m, 9H), 3.59-3.53 (m, 12H), 3.27-3.19 (m, 2H), 3.17-3.11 (m, 2H), 3.01-2.90 (m, 2H), 2.88-2.74 (m, 1H), 2.74-2.55 (m, 1H), 2.48-2.41 (m, 4H), 1.97-1.86 (m, 2H), 1.69-1.50 (m, 1H), 1.49-1.23 (m, 7H). MS (ESI) m/z 1237.6 [M+H]⁺.

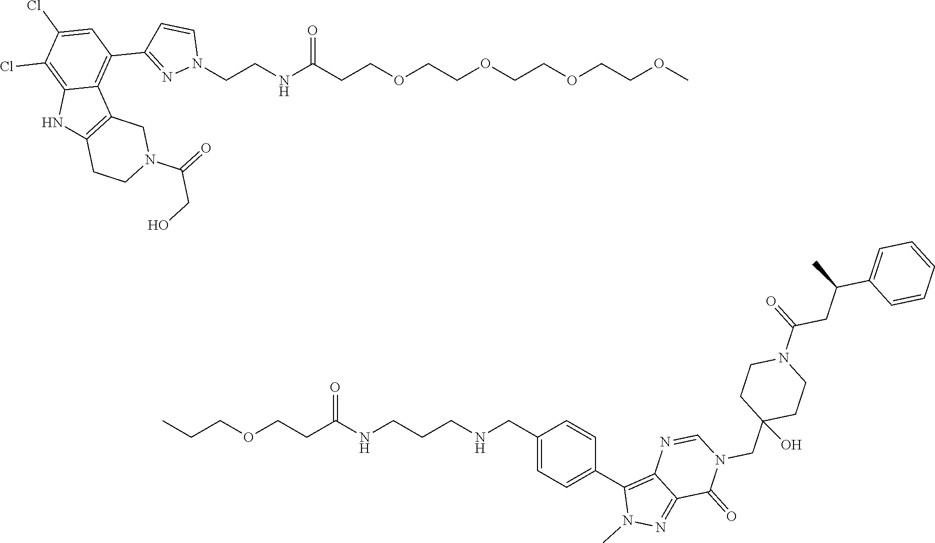

### Example 83: (R)—N1-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-N19-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)-4,7,10,13,16-pentaoxanonadecanediamide (Xh188-60)

¹H NMR (400 MHz, Methanol-d₄) δ 8.00-7.85 (m, 1H), 7.76-7.58 (m, 5H), 7.32-7.11 (m, 6H), 6.57-6.29 (m, 1H), 4.57-4.48 (m, 2H), 4.39-4.32 (m, 3H), 4.26-4.17 (m, 3H), 4.14-4.05 (m, 4H), 4.02-3.88 (m, 2H), 3.74-3.64 (m, 9H), 3.56-3.51 (m, 16H), 3.28-3.06 (m, 4H), 3.07-2.83 (m, 3H), 2.82-2.55 (m, 1H), 2.46-2.37 (m, 4H), 1.96-1.86 (m, 2H), 1.66-1.50 (m, 1H), 1.49-1.21 (m, 7H). MS (ESI) m/z 1281.6 [M+H]⁺.

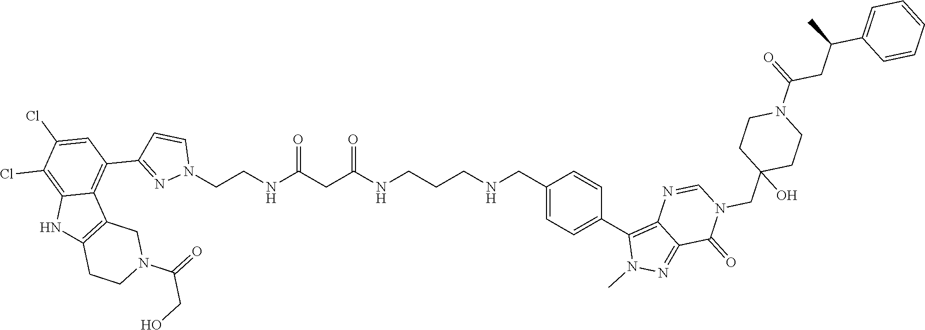 [SMILES: C[C@H](CC(=O)N1CCC(O)(Cn2cnc3c(-c4ccc(CNCCCNC(=O)CC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 84: (R)-2-(3-(6,7-dichloro-2-(2-hydroxyacetyl)-2,3,4,5-tetrahydro-1H-pyrido[4,3-b]indol-9-yl)-1H-pyrazol-1-yl)-N-(2-((3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)amino)-2-oxoethyl)acetamide (XH188-90)

¹H NMR (400 MHz, Methanol-d₄) δ 8.05-7.87 (m, 1H), 7.85-7.74 (m, 1H), 7.68-7.57 (m, 1H), 7.57-7.51 (m, 2H), 7.49-7.40 (m, 1H), 7.38-7.11 (m, 6H), 6.58-6.48 (m, 1H), 4.69 (s, 1H), 4.44 (s, 1H), 4.30 (s, 1H), 4.26-4.08 (m, 3H), 4.16-3.98 (m, 6H), 3.96-3.77 (m, 5H), 3.74-3.58 (m, 2H), 3.28-3.11 (m, 3H), 3.08-2.87 (m, 3H), 2.85-2.66 (m, 3H), 2.66-2.40 (m, 1H), 1.89-1.72 (m, 2H), 1.69-1.52 (m, 1H), 1.51-1.25 (m, 7H). MS (ESI) m/z 1033.5 [M+H]⁺.

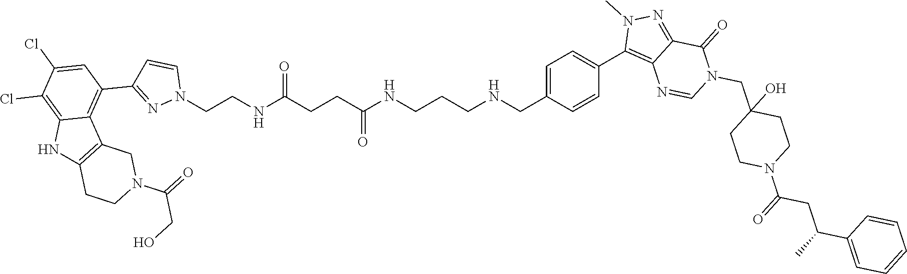 [SMILES: C[C@H](CC(=O)N1CCC(O)(Cn2cnc3c(-c4ccc(CNCCCNC(=O)CCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 85: (R)-3-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)propanamide (XH188-91)

¹H NMR (400 MHz, Methanol-d₄) δ 8.03-7.88 (m, 1H), 7.80-7.73 (m, 1H), 7.71-7.64 (m, 1H), 7.64-7.55 (m, 2H), 7.54-7.46 (m, 1H), 7.38-7.13 (m, 6H), 6.58-6.51 (m, 1H), 4.74-4.58 (m, 1H), 4.49-4.39 (m, 1H), 4.38-4.31 (m, 1H), 4.27-4.13 (m, 3H), 4.13-3.92 (m, 6H), 3.92-3.77 (m, 2H), 3.71-3.47 (m, 5H), 3.26-3.14 (m, 3H), 3.08-2.88 (m, 4H), 2.85-2.75 (m, 2H), 2.75-2.55 (m, 1H), 2.48-2.40 (m, 2H), 1.90-1.70 (m, 2H), 1.70-1.54 (m, 1H), 1.52-1.19 (m, 7H). MS (ESI) m/z 1047.5 [M+H]⁺.

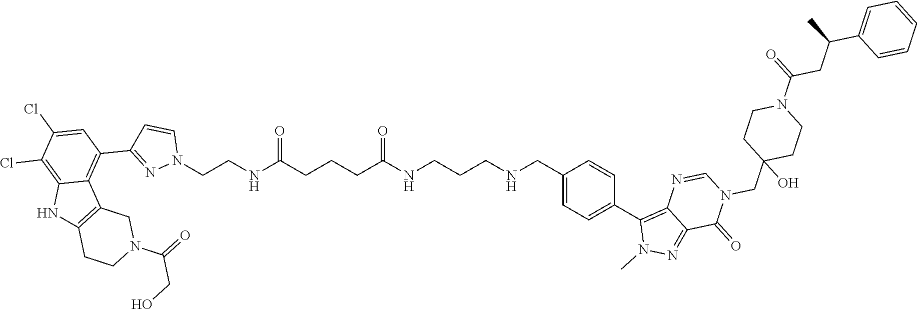 [SMILES: C[C@H](CC(=O)N1CCC(O)(Cn2cnc3c(-c4ccc(CNCCCNC(=O)CCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 86: (R)-4-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)butanamide (XH188-92)

¹H NMR (400 MHz, Methanol-d₄) δ 8.03-7.82 (m, 1H), 7.81-7.70 (m, 1H), 7.68-7.60 (m, 2H), 7.55-7.42 (m, 2H), 7.36-7.12 (m, 6H), 6.58-6.47 (m, 1H), 4.70-4.61 (m, 1H), 4.48-4.40 (m, 1H), 4.40-4.30 (m, 1H), 4.29-4.16 (m, 3H), 4.17-3.97 (m, 6H), 3.98-3.76 (m, 2H), 3.75-3.34 (m, 5H), 3.28-3.23 (m, 3H), 3.20-2.88 (m, 3H), 2.94-2.75 (m, 3H), 2.74-2.39 (m, 1H), 2.33-2.20 (m, 2H), 1.96-1.86 (m, 2H), 1.71-1.52 (m, 1H), 1.53-1.15 (m, 7H). MS (ESI) m/z 1061.5 [M+H]⁺.

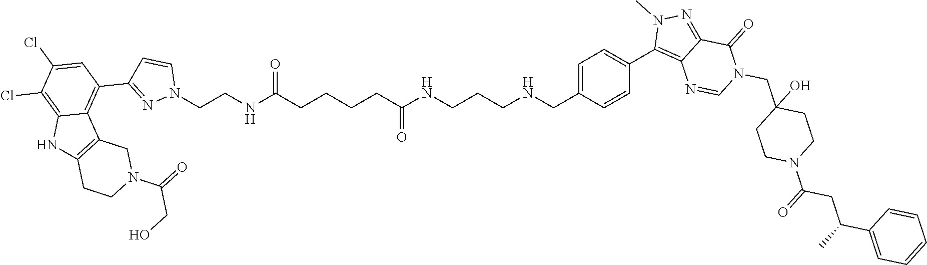 [SMILES: C[C@H](CC(=O)N1CCC(O)(Cn2cnc3c(-c4ccc(CNCCCNC(=O)CCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 87: (R)-5-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)pentanamide (XH188-93)

¹H NMR (400 MHz, Methanol-d₄) δ 8.02-7.85 (m, 1H), 7.78-7.72 (m, 1H), 7.73-7.66 (m, 1H), 7.67-7.61 (m, 2H), 7.60-7.47 (m, 1H), 7.39-7.03 (m, 6H), 6.64-6.48 (m, 1H), 4.68-4.56 (m, 1H), 4.50-4.38 (m, 1H), 4.37-4.30 (m, 1H), 4.28-4.15 (m, 3H), 4.17-3.97 (m, 6H), 3.97-3.74 (m, 2H), 3.74-3.34 (m, 5H), 3.25-3.16 (m, 3H), 3.15-2.96 (m, 3H), 2.94-2.81 (m, 2H), 2.81-2.67 (m, 1H), 2.63-2.40 (m, 1H), 2.28-2.17 (m, 2H), 1.95-1.81 (m, 2H), 1.62-1.27 (m, 12H). MS (ESI) m/z 1073.4 [M+H]⁺.

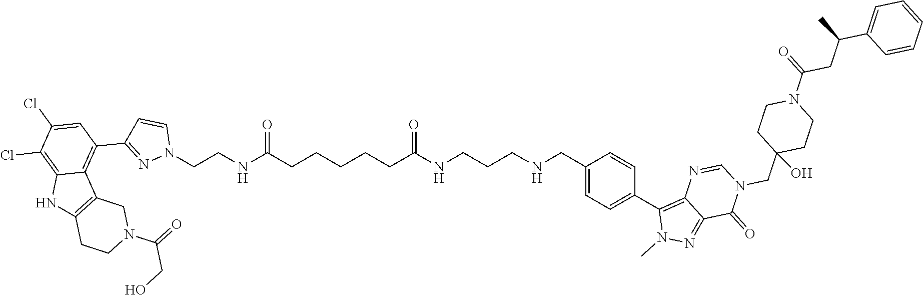 [SMILES: C[C@H](CC(=O)N1CCC(O)(Cn2cnc3c(-c4ccc(CNCCCNC(=O)CCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 88: (R)-6-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)hexanamide (XH188-94)

¹H NMR (400 MHz, Methanol-d₄) δ 7.91-7.75 (m, 1H), 7.71-7.61 (m, 1H), 7.60-7.52 (m, 2H), 7.52-7.45 (m, 1H), 7.27-7.03 (m, 6H), 6.51-6.39 (m, 1H), 4.58-4.47 (m, 1H), 4.29-4.24 (m, 1H), 4.21-4.07 (m, 3H), 4.08-3.89 (m, 6H), 3.92-3.74 (m, 2H), 3.75-3.25 (m, 5H), 3.18-3.12 (m, 3H), 3.07-2.92 (m, 3H), 2.82-2.60 (m, 3H), 2.56-2.33 (m, 1H), 2.16-2.05 (m, 2H), 1.88-1.76 (m, 2H), 1.51-1.19 (m, 14H). MS (ESI) m/z 1089.5 [M+H]⁺.

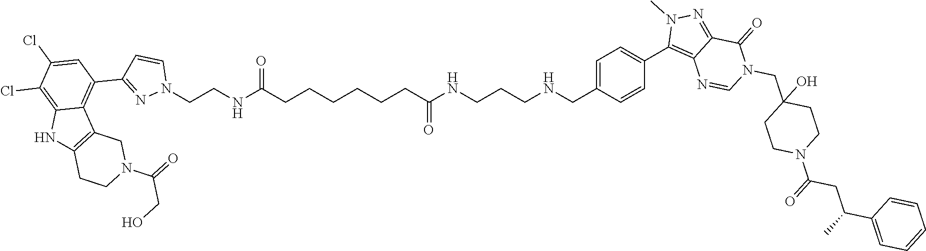 [SMILES: C[C@H](CC(=O)N1CCC(O)(Cn2cnc3c(-c4ccc(CNCCCNC(=O)CCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 89: (R)-7-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)heptanamide (XH188-95)

¹H NMR (400 MHz, Methanol-d₄) δ 8.01-7.82 (m, 1H), 7.79-7.71 (m, 1H), 7.69-7.55 (m, 4H), 7.36-7.12 (m, 6H), 6.59-6.49 (m, 1H), 4.71-4.57 (m, 1H), 4.40-4.26 (m, 2H), 4.29-4.17 (m, 3H), 4.17-4.00 (m, 6H), 3.97-3.75 (m, 2H), 3.75-3.42 (m, 5H), 3.26-3.17 (m, 3H), 3.13-2.98 (m, 3H), 2.97-2.69 (m, 3H), 2.61-2.44 (m, 1H), 2.29-2.12 (m, 2H), 2.02-1.81 (m, 2H), 1.60-1.29 (m, 16H). MS (ESI) m/z 1103.6 [M+H]⁺.

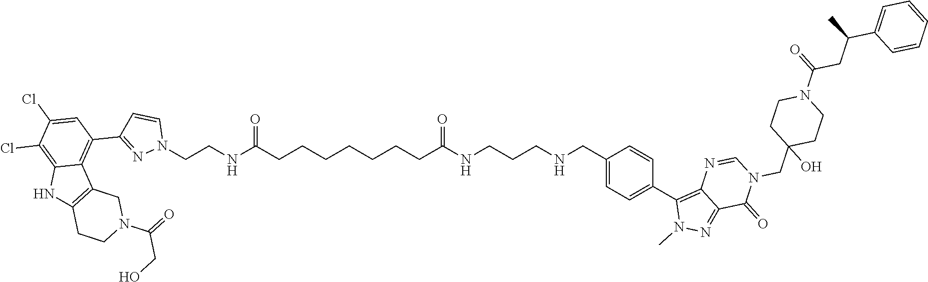 [SMILES: C[C@H](CC(=O)N1CCC(O)(Cn2cnc3c(-c4ccc(CNCCCNC(=O)CCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 90: (R)-8-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)octanamide (XH188-96)

¹H NMR (400 MHz, Methanol-d₄) δ 7.98-7.82 (m, 1H), 7.82-7.54 (m, 5H), 7.37-7.12 (m, 6H), 6.62-6.48 (m, 1H), 4.74-4.53 (m, 1H), 4.46-4.30 (m, 3H), 4.27-4.15 (m, 2H), 4.14-4.00 (m, 5H), 3.98-3.78 (m, 3H), 3.74-3.35 (m, 5H), 3.27-3.17 (m, 4H), 3.16-2.97 (m, 2H), 2.96-2.75 (m, 3H), 2.74-2.54 (m, 1H), 2.50-2.14 (m, 2H), 2.08-1.86 (m, 2H), 1.65-1.22 (m, 18H). MS (ESI) m/z 1117.5 [M+H]⁺.

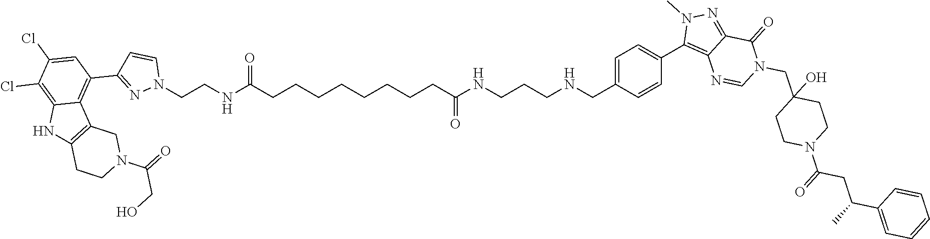 [SMILES: C[C@H](CC(=O)N1CCC(O)(Cn2cnc3c(-c4ccc(CNCCCNC(=O)CCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 91: (R)-9-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)nonanamide (XH188-97)

¹H NMR (400 MHz, Methanol-d₄) δ 8.00-7.84 (m, 1H), 7.79-7.71 (m, 1H), 7.71-7.55 (m, 4H), 7.35-7.10 (m, 6H), 6.62-6.51 (m, 1H), 4.63 (s, 1H), 4.38-4.29 (m, 3H), 4.29-4.17 (m, 2H), 4.14-3.97 (m, 5H), 3.97-3.77 (m, 2H), 3.75-3.34 (m, 5H), 3.25-3.17 (m, 3H), 3.15-2.96 (m, 3H), 2.97-2.82 (m, 2H), 2.84-2.68 (m, 1H), 2.65-2.40 (m, 1H), 2.26-2.13 (m, 2H), 1.98-1.81 (m, 2H), 1.59-1.25 (m, 20H). MS (ESI) m/z 1131.4 [M+H]⁺.

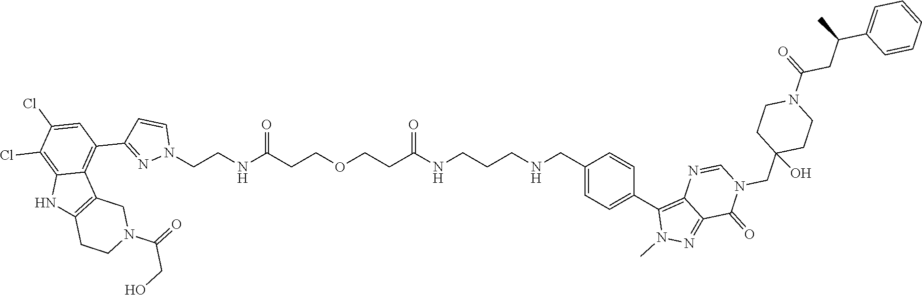 [SMILES: C[C@H](CC(=O)N1CCC(O)(Cn2cnc3c(-c4ccc(CNCCCNC(=O)CCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 92: (R)—N-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-3-(3-((3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)amino)-3-oxopropoxy)propanamide (XH188-98)

¹H NMR (400 MHz, Methanol-d₄) δ 7.97-7.79 (m, 1H), 7.78-7.72 (m, 1H), 7.67-7.58 (m, 2H), 7.58-7.46 (m, 2H), 7.36-7.11 (m, 6H), 6.61-6.48 (m, 1H), 4.62-4.53 (m, 1H), 4.48-4.36 (m, 1H), 4.38-4.28 (m, 1H), 4.27-4.15 (m, 2H), 4.12-3.95 (m, 5H), 3.98-3.81 (m, 3H), 3.79-3.54 (m, 6H), 3.58-3.39 (m, 5H), 3.26-3.16 (m, 2H), 3.15-2.91 (m, 3H), 2.91-2.76 (m, 3H), 2.71-2.56 (m, 1H), 2.54-2.38 (m, 2H), 2.04-1.81 (m, 2H), 1.68-1.52 (m, 1H), 1.50-1.20 (m, 7H). MS (ESI) m/z 1091.4 [M+H]⁺.

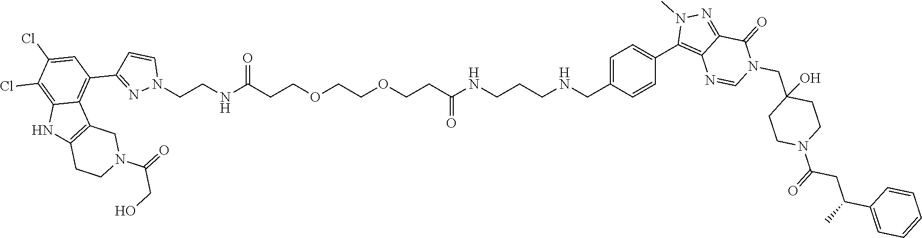 [SMILES: C[C@H](CC(=O)N1CCC(O)(Cn2cnc3c(-c4ccc(CNCCCNC(=O)CCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 93: (R)-3-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)-N-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)propanamide (XH188-99)

¹H NMR (400 MHz, Methanol-d₄) § 8.02-7.82 (m, 1H), 7.82-7.70 (m, 1H), 7.68-7.57 (m, 2H), 7.57-7.42 (m, 2H), 7.33-7.15 (m, 6H), 6.64-6.43 (m, 1H), 4.65-4.57 (m, 1H), 4.50-4.38 (m, 1H), 4.37-4.27 (m, 1H), 4.23-4.09 (m, 2H), 4.10-3.72 (m, 5H), 3.67-3.61 (m, 3H), 3.56-3.51 (m, 10H), 3.45-3.35 (m, 5H), 3.25-3.12 (m, 2H), 3.08-2.89 (m, 3H), 2.89-2.75 (m, 3H), 2.73-2.62 (m, 1H), 2.62-2.27 (m, 2H), 2.10-1.80 (m, 2H), 1.68-1.54 (m, 1H), 1.51-1.17 (m, 7H). MS (ESI) m/z 1135.5 [M+H]⁺.

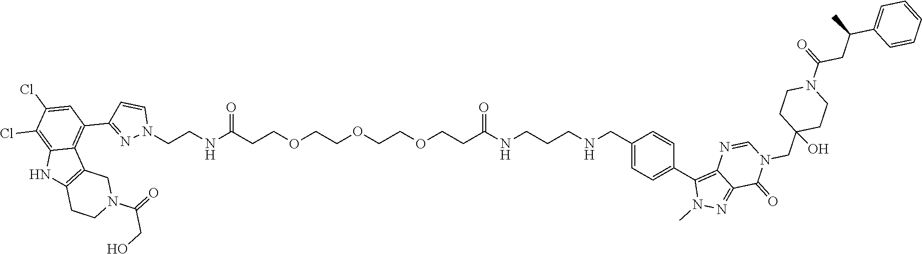 [SMILES: C[C@H](CC(=O)N1CCC(O)(Cn2cnc3c(-c4ccc(CNCCCNC(=O)CCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 94: (R)-3-(2-(2-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)ethoxy)ethoxy)-N-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)propanamide (XH188-100)

¹H NMR (400 MHz, Methanol-d₄) δ 8.02-7.83 (m, 1H), 7.81-7.71 (m, 1H), 7.70-7.61 (m, 2H), 7.61-7.56 (m, 2H), 7.38-7.12 (m, 6H), 6.61-6.51 (m, 1H), 4.68-4.55 (m, 1H), 4.46-4.37 (m, 1H), 4.36-4.27 (m, 1H), 4.24-4.18 (m, 2H), 4.13-4.00 (m, 5H), 3.98-3.78 (m, 3H), 3.74-3.63 (m, 4H), 3.59-3.51 (m, 10H), 3.50-3.34 (m, 5H), 3.28-3.14 (m, 2H), 3.15-2.96 (m, 3H), 2.96-2.78 (m, 3H), 2.76-2.54 (m, 1H), 2.49-2.43 (m, 2H), 1.99-1.82 (m, 2H), 1.65-1.50 (m, 1H), 1.45-1.26 (m, 7H). MS (ESI) m/z 1179.5 [M+H]⁺.

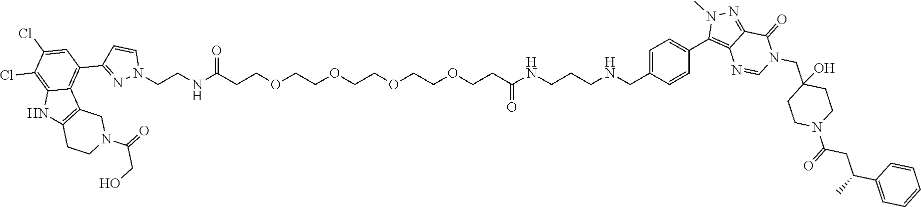 [SMILES: C[C@H](CC(=O)N1CCC(O)(Cn2cnc3c(-c4ccc(CNCCCNC(=O)CCOCCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 95: (R)-1-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)-3,6,9,12-tetraoxapentadecan-15-amide (XH188-101)

¹H NMR (400 MHz, Methanol-d₄) δ 8.01-7.83 (m, 1H), 7.82-7.73 (m, 1H), 7.73-7.57 (m, 4H), 7.36-7.11 (m, 6H), 6.61-6.49 (m, 1H), 4.68-4.57 (m, 1H), 4.47-4.38 (m, 1H), 4.35-4.27 (m, 1H), 4.23-4.18 (m, 2H), 4.14-4.01 (m, 5H), 4.00-3.77 (m, 3H), 3.79-3.62 (m, 4H), 3.60-3.49 (m, 14H), 3.46-3.33 (m, 5H), 3.27-3.17 (m, 2H), 3.17-2.97 (m, 3H), 2.96-2.75 (m, 3H), 2.74-2.54 (m, 1H), 2.50-2.42 (m, 2H), 1.93-1.81 (m, 2H), 1.67-1.52 (m, 1H), 1.53-1.15 (m, 7H). MS (ESI) m/z 1223.4 [M+H]⁺.

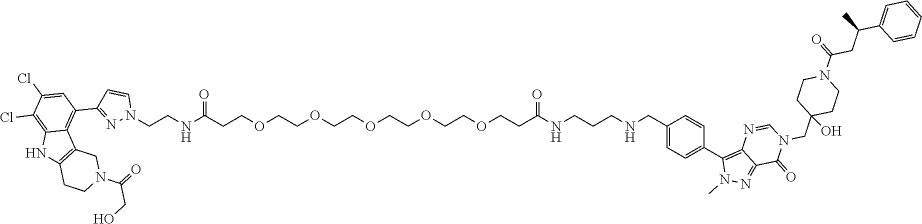 [SMILES: C[C@H](CC(=O)N1CCC(O)(Cn2cnc3c(-c4ccc(CNCCCNC(=O)CCOCCOCCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1]

### Example 96: (R)-1-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-N-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)propyl)-3,6,9,12,15-pentaoxaoctadecan-18-amide (XH188-102)

¹H NMR (400 MHz, Methanol-d₄) δ 8.02-7.83 (m, 1H), 7.80-7.73 (m, 1H), 7.71-7.56 (m, 4H), 7.36-7.10 (m, 6H), 6.61-6.51 (m, 1H), 4.67-4.58 (m, 1H), 4.46-4.38 (m, 1H), 4.35-4.29 (m, 1H), 4.25-4.18 (m, 2H), 4.14-4.02 (m, 5H), 3.99-3.77 (m, 3H), 3.77-3.62 (m, 4H), 3.62-3.49 (m, 18H), 3.46-3.32 (m, 5H), 3.28-3.17 (m, 2H), 3.17-2.96 (m, 3H), 2.95-2.76 (m, 3H), 2.75-2.54 (m, 1H), 2.50-2.42 (m, 2H), 1.93-1.83 (m, 2H), 1.64-1.51 (m, 1H), 1.48-1.19 (m, 7H). MS (ESI) m/z 1267.4 [M+H]⁻.

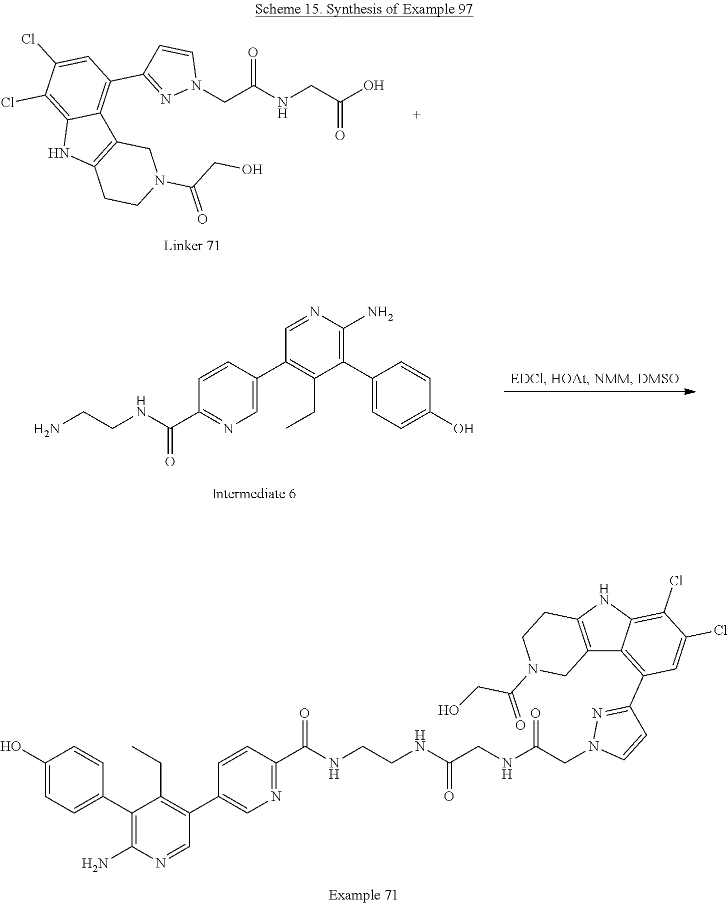 [SMILES: O=C(O)CNC(=O)Cn1ccc(-c2cc(Cl)c(Cl)c3[nH]c4c(c23)CN(C(=O)CO)CC4)n1 ; CCc1c(-c2ccc(C(=O)NCCN)nc2)cnc(N)c1-c1ccc(O)cc1 ; CCc1c(-c2ccc(C(=O)NCCNC(=O)CNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 97: 6′-amino-N-(2-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)acetamido)-ethyl)-4′-ethyl-5′-(4-hydroxyphenyl)-[3,3′-bipyridine]-6-carboxamide (KL186-107)

To a solution of intermediate 6 (3.8 mg, 0.01 mmol) in DMSO (1 mL) were added Linker 71 (4.8 mg, 0.01 mmol, 1.0 equiv), EDCI (1-ethyl-3-(3-dimethylaminopropyl)carbodiimide) (3.0 mg, 0.015 mmol, 1.5 equiv), HOAt (1-hydroxy-7-azabenzo-triazole) (2.0 mg, 0.015 mmol, 1.5 equiv), and NMM (N-Methylmorpholine) (3.0 mg, 0.03 mmol, 3.0 equiv). After being stirred overnight at room temperature, the resulting mixture was purified by preparative HPLC to afford titled compound as light yellow solid (5.3 mg, 64%). ¹H NMR (400 MHz, Methanol-d₄) δ 11.04 (s, 1H), 8.47 (s, 1H), 8.09 (t, J=7.2 Hz, 1H), 7.76 (ddd, J=10.4, 8.0, 2.1 Hz, 1H), 7.69 (dd, J=5.2, 2.3 Hz, 1H), 7.63 (s, 1H), 7.12 (s, 1H), 7.08-7.02 (m, 2H), 6.96-6.86 (m, 2H), 6.45 (t, J=2.4 Hz, 1H), 4.97 (d, J=13.6 Hz, 2H), 4.56 (s, 1H), 4.34 (s, 1H), 4.23 (s, 1H), 4.10 (s, 1H), 3.81 (d, J=6.6 Hz, 3H), 3.60 (t, J=5.8 Hz, 1H), 3.43 (t, J=5.7 Hz, 1H), 3.33 (dt, J=17.0, 5.6 Hz, 2H), 3.27-3.22 (m, 1H), 2.85 (t, J=5.7 Hz, 1H), 2.77 (t, J=5.8 Hz, 1H), 2.29-2.23 (m, 2H), 0.56-0.51 (m, 3H). MS (ESI) m/z 839.3 [M+H]⁻.

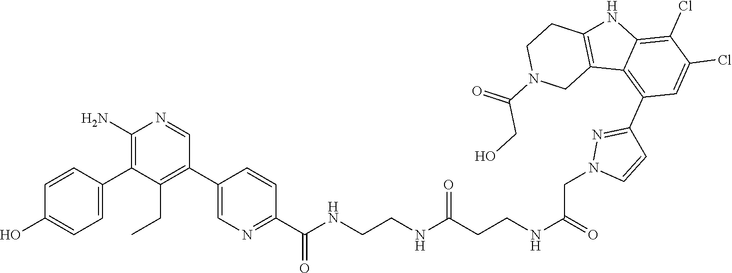 [SMILES: CCc1c(-c2ccc(C(=O)NCCNC(=O)CCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 98: 6′-amino-N-(2-(3-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)propane-mido)ethyl)-4′-ethyl-5′-(4-hydroxyphenyl)-[3,3′-bipyridine]-6-carboxamide (KL186-115)

Example 98 was synthesized following similar procedure for preparing example 97 from intermediate 6 and linker 72. Light yellow solid, 5.5 mg, 65% yield. ¹H NMR (400 MHz, Methanol-d₄) δ 8.50 (d, J=24.1 Hz, 1H), 8.07 (dd, J=21.4, 8.0 Hz, 1H), 7.78-7.68 (m, 1H), 7.64 (t, J=2.7 Hz, 1H), 7.55 (d, J=11.6 Hz, 1H), 7.09 (d, J=5.6 Hz, 1H), 7.08-7.00 (m, 2H), 6.90 (dd, J=8.5, 1.9 Hz, 2H), 6.44 (dd, J=6.8, 2.3 Hz, 1H), 4.90 (s, 1H), 4.84 (s, 1H), 4.56 (s, 1H), 4.36 (s, 1H), 4.25 (s, 1H), 4.12 (s, 1H), 3.80 (t, J=5.8 Hz, 1H), 3.59 (t, J=5.7 Hz, 1H), 3.44-3.35 (m, 4H), 3.30 (d, J=5.8 Hz, 2H), 2.80 (dt, J=30.7, 5.7 Hz, 2H), 2.30 (q, J=5.9 Hz, 2H), 2.27-2.12 (m, 2H), 0.50 (dt, J=7.8, 3.8 Hz, 3H). MS (ESI) m/z 853.3 [M+H]⁺.

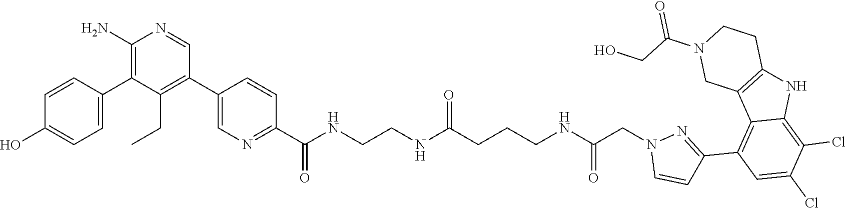 [SMILES: CCc1c(-c2ccc(C(=O)NCCNC(=O)CCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 99: 6′-amino-N-(2-(4-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)butanamido)-ethyl)-4′-ethyl-5′-(4-hydroxyphenyl)-[3,3′-bipyridine]-6-carboxamide (KL186-116)

Example 99 was synthesized following similar procedure for preparing example 97 from intermediate 6 and linker 73. Light yellow solid, 5.0 mg, 58% yield. ¹H NMR (400 MHz, Methanol-d₄) δ 11.04 (s, 1H), 8.49 (dd, J=12.4, 2.0 Hz, 1H), 8.04 (t, J=8.2 Hz, 1H), 7.79 (ddd, J=12.1, 8.1, 2.2 Hz, 1H), 7.74-7.53 (m, 2H), 7.11 (s, 1H), 7.08-6.96 (m, 2H), 6.96-6.82 (m, 2H), 6.45 (t, J=2.5 Hz, 1H), 4.84 (d, J=7.0 Hz, 2H), 4.57 (s, 1H), 4.34 (s, 1H), 4.25 (s, 1H), 4.12 (s, 1H), 3.82 (t, J=5.9 Hz, 1H), 3.62 (t, J=5.7 Hz, 1H), 3.43 (t, J=5.8 Hz, 2H), 3.32 (q, J=7.3, 6.6 Hz, 2H), 3.16-3.08 (m, 2H), 2.86 (s, 1H), 2.82-2.73 (m, 1H), 2.29-2.22 (m, 2H), 2.12 (q, J=7.6 Hz, 2H), 1.73-1.64 (m, 2H), 0.55-0.50 (m, 3H). MS (ESI) m/z 867.3 [M+H]⁺.

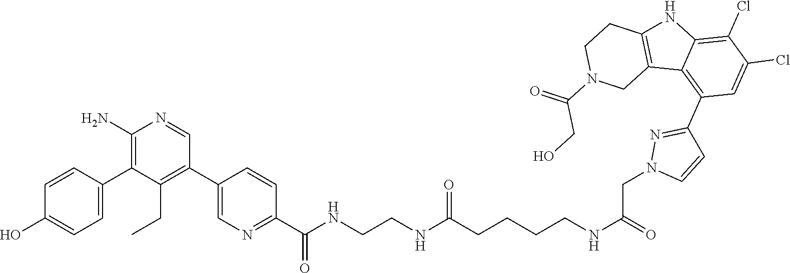 [SMILES: CCc1c(-c2ccc(C(=O)NCCNC(=O)CCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 100: 6′-amino-N-(2-(5-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)-pentan-amido)ethyl)-4′-ethyl-5′-(4-hydroxyphenyl)-[3,3′-bipyridine]-6-carboxamide (KL186-117)

Example 100 was synthesized following similar procedure for preparing example 97 from intermediate 6 and linker 74. Light yellow solid, 5.5 mg, 65% yield. ¹H NMR (400 MHz, Methanol-d₄) δ 8.50 (d, J=2.1 Hz, 1H), 8.05 (t, J=7.1 Hz, 1H), 7.79 (ddd, J=10.4, 8.0, 2.2 Hz, 1H), 7.63 (dd, J=16.1, 2.9 Hz, 2H), 7.10 (d, J=3.3 Hz, 1H), 7.04 (td, J=6.7, 2.2 Hz, 2H), 6.92-6.86 (m, 2H), 6.45 (t, J=2.3 Hz, 1H), 4.83 (d, J=14.1 Hz, 2H), 4.57 (s, 1H), 4.35 (s, 1H), 4.26 (s, 1H), 4.12 (s, 1H), 3.83 (t, J=5.8 Hz, 1H), 3.63 (t, J=5.7 Hz, 1H), 3.45 (q, J=5.3 Hz, 2H), 3.35 (d, J=5.8 Hz, 2H), 3.11-3.00 (m, 2H), 2.91-2.75 (m, 2H), 2.26 (q, J=7.1 Hz, 2H), 2.10 (q, J=6.7 Hz, 2H), 1.54-1.47 (m, 2H), 1.44-1.34 (m, 2H), 0.56-0.50 (m, 3H). MS (ESI) m/z 881.3 [M+H]⁺.

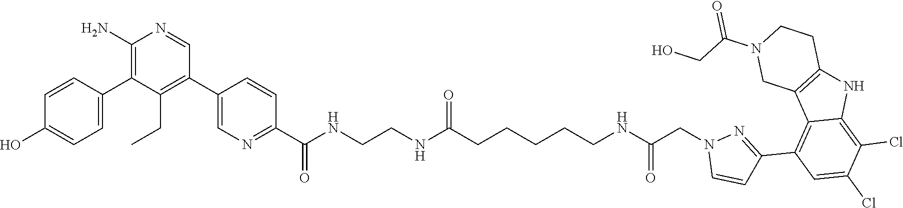 [SMILES: CCc1c(-c2ccc(C(=O)NCCNC(=O)CCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 101: 6′-amino-N-(2-(6-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)hexan-amido)ethyl)-4′-ethyl-5′-(4-hydroxyphenyl)-[3,3′-bipyridine]-6-carboxamide (KL186-118)

Example 101 was synthesized following similar procedure for preparing example 97 from intermediate 6 and linker 75. Light yellow solid, 4.8 mg, 54% yield. ¹H NMR (400 MHz, Methanol-d₄) δ 11.04 (s, 1H), 8.51 (d, J=2.2 Hz, 1H), 8.05 (dd, J=9.9, 7.9 Hz, 1H), 7.80 (ddd, J=17.0, 8.1, 2.2 Hz, 1H), 7.64 (dd, J=9.7, 3.2 Hz, 2H), 7.11 (d, J=2.9 Hz, 1H), 7.09-6.98 (m, 2H), 6.90 (d, J=8.2 Hz, 2H), 6.45 (d, J=2.3 Hz, 1H), 4.83 (d, J=10.2 Hz, 2H), 4.57 (s, 1H), 4.35 (s, 1H), 4.25 (s, 1H), 4.13 (s, 1H), 3.83 (t, J=5.8 Hz, 1H), 3.62 (t, J=5.7 Hz, 1H), 3.46 (dd, J=6.5, 3.8 Hz, 2H), 3.35 (dd, J=6.5, 4.0 Hz, 2H), 3.10-2.99 (m, 2H), 2.82 (dt, J=28.7, 5.7 Hz, 2H), 2.30-2.23 (m, 2H), 2.09-2.03 (m, 2H), 1.52-1.30 (m, 4H), 1.24-1.16 (m, 2H), 0.53 (q, J=7.4 Hz, 3H). MS (ESI) m/z 895.3 [M+H]⁺.

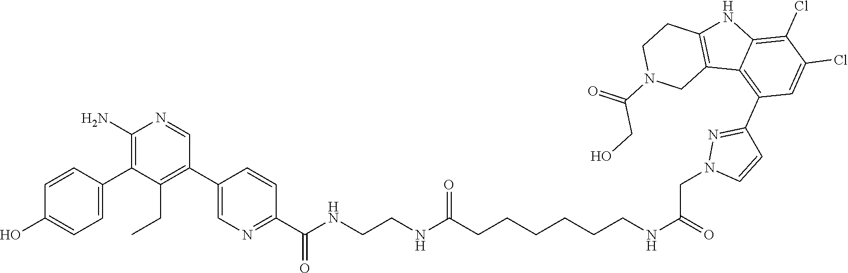 [SMILES: CCc1c(-c2ccc(C(=O)NCCNC(=O)CCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 102: 6′-amino-N-(2-(7-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)heptan-amido)ethyl)-4′-ethyl-5′-(4-hydroxyphenyl)-[3,3′-bipyridine]-6-carboxamide (KL186-119)

Example 102 was synthesized following similar procedure for preparing example 97 from intermediate 6 and linker 76. Light yellow solid, 5.5 mg, 61% yield. ¹H NMR (400 MHz, Methanol-d₄) δ 8.48 (d, J=2.3 Hz, 1H), 8.05 (t, J=8.0 Hz, 1H), 7.81 (ddd, J=21.1, 8.0, 2.2 Hz, 1H), 7.71-7.56 (m, 2H), 7.11 (s, 1H), 7.08-7.00 (m, 2H), 6.90 (dd, J=8.6, 2.1 Hz, 2H), 6.46 (d, J=2.3 Hz, 1H), 4.84 (d, J=5.0 Hz, 2H), 4.59 (s, 1H), 4.35 (s, 1H), 4.25 (s, 1H), 4.12 (s, 1H), 3.84 (t, J=5.9 Hz, 1H), 3.62 (t, J=5.7 Hz, 1H), 3.53-3.40 (m, 2H), 3.41-3.29 (m, 2H), 3.06 (dt, J=24.0, 6.9 Hz, 2H), 2.89-2.75 (m, 2H), 2.34-2.21 (m, 2H), 2.09-2.02 (m, 2H), 1.52-1.24 (m, 4H), 1.24-1.07 (m, 4H), 0.57-0.51 (m, 3H). MS (ESI) m/z 909.3 [M+H]⁺.

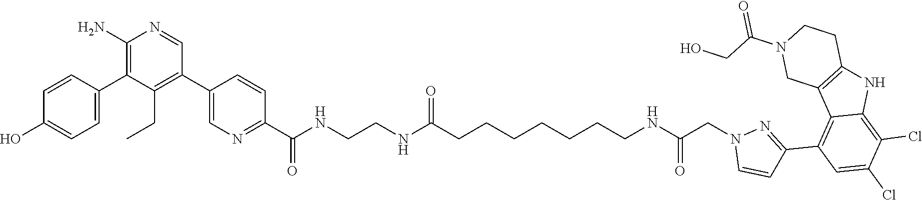 [SMILES: CCc1c(-c2ccc(C(=O)NCCNC(=O)CCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 103: 6′-amino-N-(2-(8-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)octanamido)-ethyl)-4′-ethyl-5′-(4-hydroxyphenyl)-[3,3′-bipyridine]-6-carboxamide (KL186-120)

Example 103 was synthesized following similar procedure for preparing example 97 from intermediate 6 and linker 77. Light yellow solid, 5.5 mg, 60% yield. ¹H NMR (400 MHz, Methanol-d₄) δ 8.52-8.41 (m, 1H), 8.06 (dd, J=8.2, 3.3 Hz, 1H), 7.81 (ddd, J=16.7, 8.1, 2.2 Hz, 1H), 7.66 (d, J=2.3 Hz, 1H), 7.59 (d, J=3.5 Hz, 1H), 7.12 (d, J=1.5 Hz, 1H), 7.09-6.96 (m, 2H), 6.90 (dd, J=8.6, 2.9 Hz, 2H), 6.46 (d, J=2.3 Hz, 1H), 4.84 (d, J=5.4 Hz, 2H), 4.57 (s, 1H), 4.36 (s, 1H), 4.23 (d, J=3.7 Hz, 1H), 4.12 (d, J=3.4 Hz, 1H), 3.85 (t, J=5.8 Hz, 1H), 3.62 (d, J=6.3 Hz, 1H), 3.47 (d, J=6.0 Hz, 2H), 3.41-3.33 (m, 2H), 3.15-3.01 (m, 2H), 2.91-2.76 (m, 2H), 2.27 (q, J=7.5 Hz, 2H), 2.09-2.02 (m, 2H), 1.45-1.33 (m, 4H), 1.12 (d, J=14.1 Hz, 6H), 0.54 (t, J=7.5 Hz, 3H). MS (ESI) m/z 923.4 [M+H]⁺.

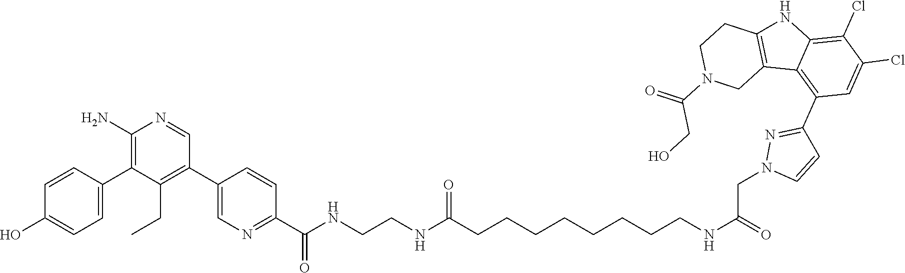 [SMILES: CCc1c(-c2ccc(C(=O)NCCNC(=O)CCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 104: 6′-amino-N-(2-(9-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)nonan-amido)ethyl)-4′-ethyl-5′-(4-hydroxyphenyl)-[3,3′-bipyridine]-6-carboxamide (KL186-121)

Example 104 was synthesized following similar procedure for preparing example 97 from intermediate 6 and linker 78. Light yellow solid, 5.3 mg, 57% yield. ¹H NMR (400 MHz, Methanol-d₄) δ 8.46 (dd, J=7.1, 2.1 Hz, 1H), 8.05 (t, J=8.3 Hz, 1H), 7.78 (ddd, J=32.9, 8.0, 2.2 Hz, 1H), 7.66 (t, J=2.1 Hz, 1H), 7.57 (d, J=16.2 Hz, 1H), 7.12 (s, 1H), 7.10-6.98 (m, 2H), 6.90 (dd, J=8.6, 2.6 Hz, 2H), 6.46 (d, J=2.3 Hz, 1H), 4.85 (s, 2H), 4.60 (s, 1H), 4.36 (s, 1H), 4.22 (s, 1H), 4.12 (s, 1H), 3.84 (t, J=5.8 Hz, 1H), 3.62 (t, J=5.8 Hz, 1H), 3.53-3.44 (m, 2H), 3.37 (t, J=5.1 Hz, 2H), 3.11 (q, J=7.2 Hz, 2H), 2.91-2.75 (m, 2H), 2.31-2.23 (m, 2H), 2.06 (q, J=7.8 Hz, 2H), 1.46-1.32 (m, 4H), 1.09 (d, J=27.4 Hz, 8H), 0.55 (q, J=7.7 Hz, 3H). MS (ESI) m/z 937.4 [M+H]⁺.

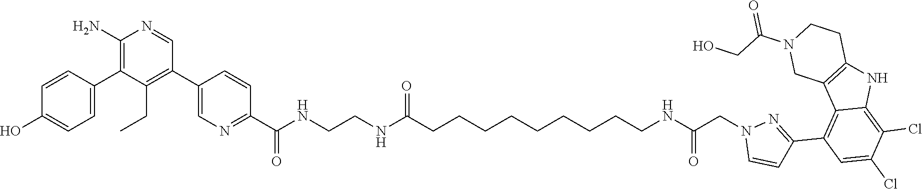 [SMILES: CCc1c(-c2ccc(C(=O)NCCNC(=O)CCCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 105: 6′-amino-N-(2-(10-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)decan-amido)ethyl)-4′-ethyl-5′-(4-hydroxyphenyl)-[3,3′-bipyridine]-6-carboxamide (KL186-122)

Example 105 was synthesized following similar procedure for preparing example 97 from intermediate 6 and linker 79. Light yellow solid, 6.1 mg, 64% yield. ¹H NMR (400 MHz, Methanol-d₄) δ 8.48 (dd, J=6.9, 2.1 Hz, 1H), 8.06 (t, J=7.4 Hz, 1H), 7.81 (ddd, J=27.3, 8.1, 2.2 Hz, 1H), 7.66 (d, J=2.3 Hz, 1H), 7.59 (d, J=12.1 Hz, 1H), 7.11 (d, J=1.1 Hz, 1H), 7.08-6.99 (m, 2H), 6.90 (dd, J=8.5, 3.4 Hz, 2H), 6.46 (d, J=2.3 Hz, 1H), 4.85 (s, 2H), 4.58 (s, 1H), 4.35 (s, 1H), 4.22 (s, 1H), 4.12 (s, 1H), 3.84 (t, J=5.8 Hz, 1H), 3.62 (t, J=5.8 Hz, 1H), 3.47 (dd, J=6.8, 4.7 Hz, 2H), 3.36 (dd, J=6.8, 4.7 Hz, 2H), 3.18-3.07 (m, 2H), 2.83 (dt, J=25.0, 5.8 Hz, 2H), 2.32-2.24 (m, 2H), 2.09-2.04 (m, 2H), 1.46-1.33 (m, 4H), 1.11 (s, 10H), 0.56 (q, J=7.8 Hz, 3H). MS (ESI) m/z 951.4 [M+H]⁺.

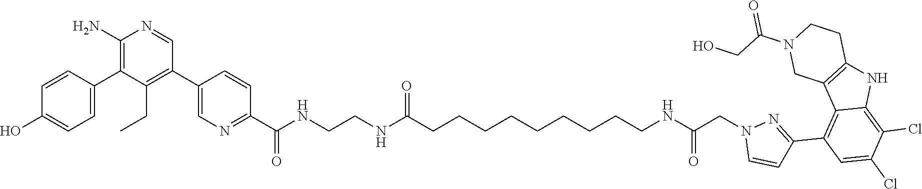 [SMILES: CCc1c(-c2ccc(C(=O)NCCNC(=O)CCCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 106: 6′-amino-N-(2-(11-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)undecane-amido)ethyl)-4′-ethyl-5′-(4-hydroxyphenyl)-[3,3′-bipyridine]-6-carboxamide (KL186-123)

Example 106 was synthesized following similar procedure for preparing example 97 from intermediate 6 and linker 80. Light yellow solid, 5.6 mg, 58% yield. ¹H NMR (400 MHz, Methanol-d₄) δ 8.49 (dd, J=10.0, 2.2 Hz, 1H), 8.07 (t, J=8.1 Hz, 1H), 7.82 (ddd, J=33.9, 8.0, 2.2 Hz, 1H), 7.66 (t, J=1.9 Hz, 1H), 7.61 (d, J=11.6 Hz, 1H), 7.12 (s, 1H), 7.09-6.97 (m, 2H), 6.90 (dd, J=8.6, 3.4 Hz, 2H), 6.46 (d, J=2.3 Hz, 1H), 4.85 (s, 2H), 4.59 (s, 1H), 4.35 (s, 1H), 4.22 (s, 1H), 4.12 (s, 1H), 3.84 (t, J=5.8 Hz, 1H), 3.62 (t, J=5.7 Hz, 1H), 3.47 (dd, J=6.8, 4.7 Hz, 2H), 3.36 (dd, J=6.8, 4.7 Hz, 2H), 3.12 (q, J=7.3 Hz, 2H), 2.83 (dt, J=24.9, 5.8 Hz, 2H), 2.36-2.21 (m, 2H), 2.09-2.05 (m, 2H), 1.47-1.33 (m, 4H), 1.19-1.04 (m, 12H), 0.60-0.54 (m, 3H). MS (ESI) m/z 965.4 [M+H]⁺.

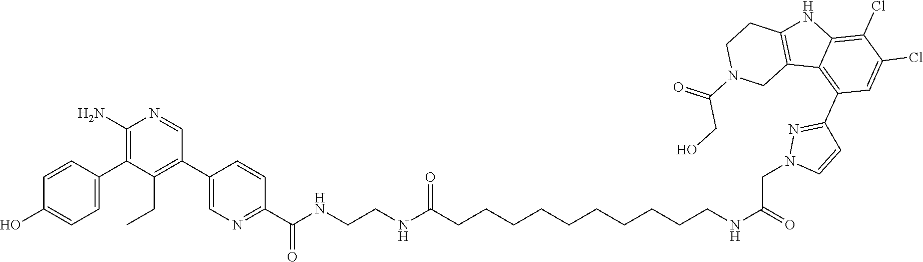 [SMILES: CCc1c(-c2ccc(C(=O)NCCNC(=O)CCCCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 107: 6′-amino-N-(2-(3-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)propanamido)ethyl)-4′-ethyl-5′-(4-hydroxyphenyl)-[3,3′-bipyridine]-6-carboxamide (KL186-124)

Example 107 was synthesized following similar procedure for preparing example 97 from intermediate 6 and linker 81. Light yellow solid, 4.9 mg, 55% yield. ¹H NMR (400 MHz, Methanol-d₄) δ 8.56-8.44 (m, 1H), 8.04 (t, J=8.6 Hz, 1H), 7.76 (ddd, J=18.9, 8.0, 2.2 Hz, 1H), 7.66 (t, J=2.8 Hz, 1H), 7.61 (d, J=4.7 Hz, 1H), 7.10 (d, J=6.6 Hz, 1H), 7.04 (dd, J=8.4, 5.8 Hz, 2H), 6.90 (d, J=8.2 Hz, 2H), 6.45 (dd, J=6.4, 2.3 Hz, 1H), 4.87 (d, J=18.2 Hz, 2H), 4.55 (s, 1H), 4.37 (s, 1H), 4.25 (s, 1H), 4.14 (s, 1H), 3.83 (t, J=5.9 Hz, 1H), 3.64-3.54 (m, 3H), 3.48-3.44 (m, 2H), 3.39-3.34 (m, 4H), 3.25 (d, J=5.2 Hz, 2H), 2.92-2.73 (m, 2H), 2.43-2.13 (m, 4H), 0.54-0.49 (m, 3H). MS (ESI) m/z 897.3 [M+H]⁻.

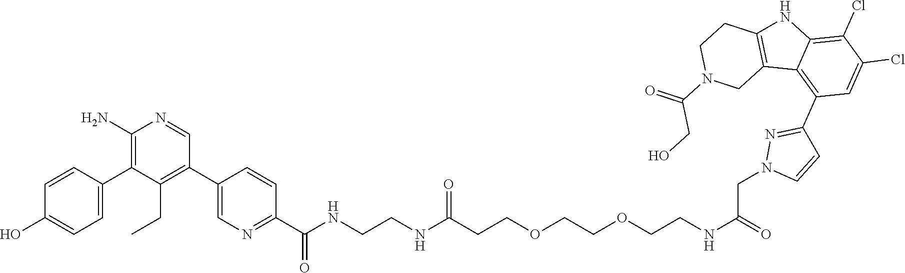 [SMILES: CCc1c(-c2ccc(C(=O)NCCNC(=O)CCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 108: 6′-amino-N-(1-(3-(6,7-dichloro-2-(2-hydroxyacetyl)-2,3,4,5-tetrahydro-1H-pyrido[4,3-b]indol-9-yl)-1H-pyrazol-1-yl)-2,12-dioxo-6,9-dioxa-3,13-diazapentadecan-15-yl)-4′-ethyl-5′-(4-hydroxyphenyl)-[3,3′-bipyridine]-6-carboxamide (KL186-125)

Example 108 was synthesized following similar procedure for preparing example 97 from intermediate 6 and linker 82. Light yellow solid, 5.9 mg, 63% yield. ¹H NMR (400 MHz, Methanol-d₄) δ 8.53-8.39 (m, 1H), 8.04 (d, J=8.0 Hz, 1H), 7.79 (dd, J=8.0, 2.1 Hz, 1H), 7.67 (d, J=2.3 Hz, 1H), 7.61 (d, J=10.2 Hz, 1H), 7.11 (d, J=2.8 Hz, 1H), 7.03 (dd, J=8.5, 3.1 Hz, 2H), 6.94-6.83 (m, 2H), 6.46 (dd, J=4.9, 2.3 Hz, 1H), 4.88 (d, J=14.2 Hz, 2H), 4.58 (s, 1H), 4.40 (s, 1H), 4.23 (s, 1H), 4.13 (s, 1H), 3.91-3.73 (m, 3H), 3.62 (t, J=5.6 Hz, 1H), 3.54-3.45 (m, 5H), 3.45-3.35 (m, 4H), 3.32 (t, J=5.3 Hz, 1H), 3.25 (t, J=5.3 Hz, 4H), 2.93-2.73 (m, 2H), 2.25 (q, J=7.6 Hz, 2H), 0.63-0.40 (m, 3H). MS (ESI) m/z 943.9 [M+H]⁻.

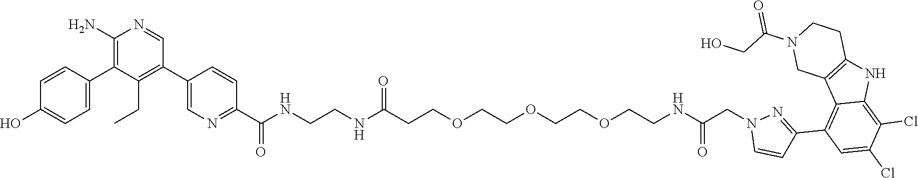 [SMILES: CCc1c(-c2ccc(C(=O)NCCNC(=O)CCOCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 109: 6′-amino-N-(1-(3-(6,7-dichloro-2-(2-hydroxyacetyl)-2,3,4,5-tetrahydro-1H-pyrido[4,3-b]indol-9-yl)-1H-pyrazol-1-yl)-2,15-dioxo-6,9,12-trioxa-3,16-diazaoctadecan-18-yl)-4′-ethyl-5′-(4-hydroxyphenyl)-[3,3′-bipyridine]-6-carboxamide (KL186-126)

Example 109 was synthesized following similar procedure for preparing example 97 from intermediate 6 and linker 83. Light yellow solid, 6.0 mg, 61% yield. ¹H NMR (400 MHz, Methanol-d₄) δ 8.49 (d, J=2.2 Hz, 1H), 8.05 (t, J=7.7 Hz, 1H), 7.81 (ddd, J=18.2, 8.1, 2.2 Hz, 1H), 7.67 (d, J=2.3 Hz, 1H), 7.63 (s, 1H), 7.12 (d, J=1.3 Hz, 1H), 7.08-7.01 (m, 2H), 6.90 (dd, J=8.7, 1.9 Hz, 2H), 6.46 (dd, J=5.8, 2.3 Hz, 1H), 4.88 (d, J=9.5 Hz, 2H), 4.57 (s, 1H), 4.39 (s, 1H), 4.23 (s, 1H), 4.13 (s, 1H), 3.84 (t, J=5.8 Hz, 1H), 3.63-3.55 (m, 3H), 3.49-3.29 (m, 16H), 2.90-2.74 (m, 2H), 2.35-2.26 (m, 4H), 0.57-0.53 (m, 3H). MS (ESI) m/z 985.4 [M+H]⁺.

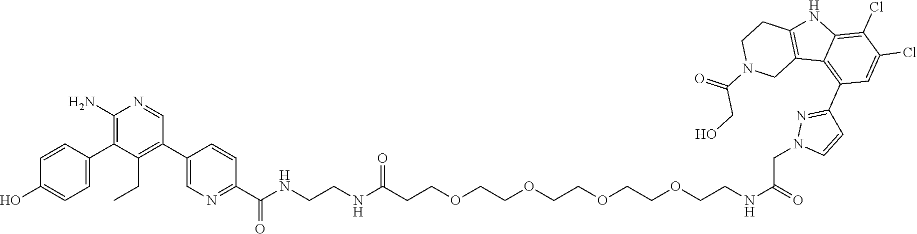 [SMILES: CCc1c(-c2ccc(C(=O)NCCNC(=O)CCOCCOCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 110: 6′-amino-N-(1-(3-(6,7-dichloro-2-(2-hydroxyacetyl)-2,3,4,5-tetrahydro-1H-pyrido[4,3-b]indol-9-yl)-1H-pyrazol-1-yl)-2,18-dioxo-6,9,12,15-tetraoxa-3,19-diazahenicosan-21-yl)-4′-ethyl-5′-(4-hydroxyphenyl)-[3,3′-bipyridine]-6-carboxamide (KL186-127)

Example 110 was synthesized following similar procedure for preparing example 97 from intermediate 6 and linker 84. Light yellow solid, 5.8 mg, 56% yield. ¹H NMR (400 MHz, Methanol-d₄) δ 8.62 (s, 1H), 8.18 (s, 1H), 7.97 (d, J=9.4 Hz, 1H), 7.77 (d, J=12.8 Hz, 2H), 7.24 (s, 1H), 7.21-7.12 (m, 2H), 7.02 (dd, J=8.6, 2.4 Hz, 2H), 6.59 (s, 1H), 5.00 (d, J=7.4 Hz, 2H), 4.69 (s, 1H), 4.50 (s, 1H), 4.35 (s, 1H), 4.25 (s, 1H), 3.96 (t, J=5.8 Hz, 1H), 3.75-3.69 (m, 3H), 3.60-3.52 (m, 16H), 3.48-3.39 (m, 4H), 3.04-2.85 (m, 2H), 2.48-2.38 (m, 4H), 0.74-0.63 (m, 3H). MS (ESI) m/z 1029.4 [M+H]⁺.

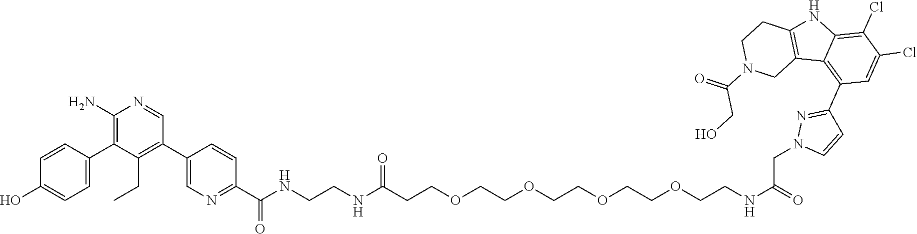 [SMILES: CCc1c(-c2ccc(C(=O)NCCNC(=O)CCOCCOCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 111: 6′-amino-N-(1-(3-(6,7-dichloro-2-(2-hydroxyacetyl)-2,3,4,5-tetrahydro-1H-pyrido[4,3-b]indol-9-yl)-1H-pyrazol-1-yl)-2,21-dioxo-6,9,12,15,18-pentaoxa-3,22-diazatetracosan-24-yl)-4′-ethyl-5′-(4-hydroxyphenyl)-[3,3′-bipyridine]-6-carboxamide (KL186-128)

Example 111 was synthesized following similar procedure for preparing example 97 from intermediate 6 and linker 85. Light yellow solid, 6.8 mg, 63% yield. ¹H NMR (400 MHz, Methanol-d₄) δ 8.51 (d, J=5.6 Hz, 1H), 8.06 (dd, J=8.2, 3.8 Hz, 1H), 7.87-7.81 (m, 1H), 7.65 (dd, J=7.9, 2.9 Hz, 2H), 7.12 (s, 1H), 7.10-6.99 (m, 2H), 6.90 (dd, J=8.6, 2.5 Hz, 2H), 6.46 (dd, J=6.2, 2.4 Hz, 1H), 4.88 (d, J=7.3 Hz, 2H), 4.57 (s, 1H), 4.38 (s, 1H), 4.23 (s, 1H), 4.12 (s, 1H), 3.84 (t, J=5.8 Hz, 1H), 3.63-3.56 (m, 3H), 3.49-3.38 (m, 20H), 3.36-3.27 (m, 4H), 2.88-2.76 (m, 2H), 2.34-2.25 (m, 4H), 0.59-0.54 (m, 3H). MS (ESI) m/z 1073.4 [M+H]⁺.

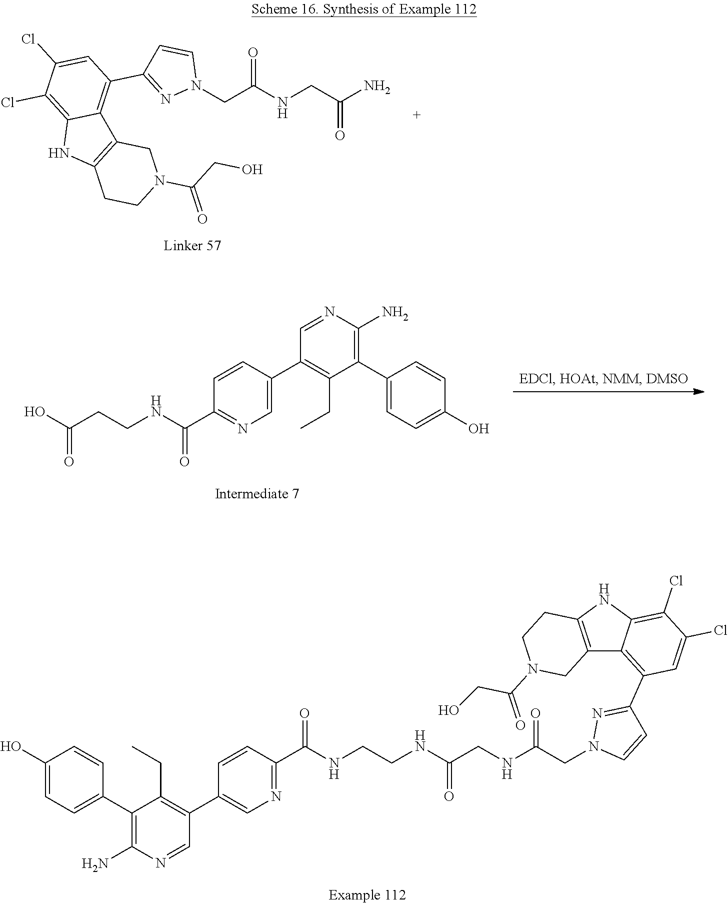 [SMILES: NC(=O)CNC(=O)Cn1ccc(-c2cc(Cl)c(Cl)c3[nH]c4c(c23)CN(C(=O)CO)CC4)n1 ; CCc1c(-c2ccc(C(=O)NCCC(=O)O)nc2)cnc(N)c1-c1ccc(O)cc1 ; CCc1c(-c2ccc(C(=O)NCCNC(=O)CNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 112: 6′-amino-N-(3-((2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethyl)amino)-3-oxopropyl)-4′-ethyl-5′-(4-hydroxyphenyl)-[3,3′-bipyridine]-6-carboxamide (KL186-129)

To a solution of intermediate 7 (4 mg, 0.01 mmol) in DMSO (1 mL) were added linker 57 (4.6 mg, 0.01 mmol, 1.0 equiv), EDCI (1-ethyl-3-(3-dimethylaminopropyl)carbodiimide) (3.0 mg, 0.015 mmol, 1.5 equiv), HOAt (1-hydroxy-7-azabenzo-triazole) (2.0 mg, 0.015 mmol, 1.5 equiv), and NMM (N-Methylmorpholine) (3.0 mg, 0.03 mmol, 3.0 equiv). After being stirred overnight at room temperature, the resulting mixture was purified by preparative HPLC to afford titled compound as light yellow solid (4.9 mg, 57%). ¹H NMR (400 MHz, Methanol-d₄) δ 8.46 (t, J=2.9 Hz, 1H), 8.04 (t, J=7.2 Hz, 1H), 7.76 (dt, J=8.1, 2.4 Hz, 1H), 7.65 (q, J=3.4, 2.8 Hz, 1H), 7.59 (d, J=6.3 Hz, 1H), 7.08 (d, J=1.7 Hz, 1H), 7.04-7.01 (m, 2H), 6.90 (dt, J=8.4, 2.1 Hz, 2H), 6.44 (dd, J=6.9, 2.4 Hz, 1H), 4.85 (d, J=14.9 Hz, 2H), 4.56 (s, 1H), 4.37 (s, 1H), 4.24 (s, 1H), 4.12 (s, 1H), 3.81 (t, J=5.9 Hz, 1H), 3.61-3.50 (m, 3H), 3.24 (d, J=3.6 Hz, 4H), 2.81 (dt, J=30.8, 5.7 Hz, 2H), 2.46-2.33 (m, 2H), 2.24 (q, J=7.5 Hz, 2H), 0.52 (t, J=7.5 Hz, 3H). MS (ESI) m/z 853.4 [M+H]⁺.

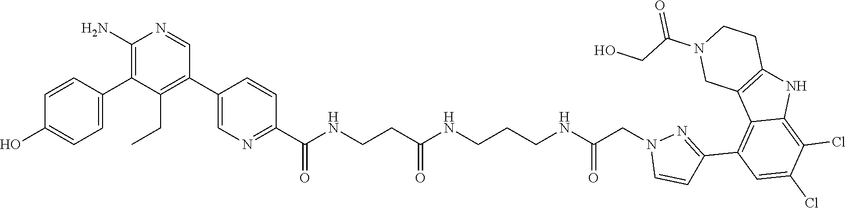 [SMILES: CCc1c(-c2ccc(C(=O)NCCC(=O)NCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 113: 6′-amino-N-(3-((3-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)propyl)-amino)-3-oxopropyl)-4′-ethyl-5′-(4-hydroxyphenyl)-[3,3′-bipyridine]-6-carboxamide (KL186-130)

Example 113 was synthesized following similar procedure for preparing example 112 from intermediate 7 and linker 58. Light yellow solid, 4.6 mg, 53% yield. ¹H NMR (400 MHz, Methanol-d₄) δ 8.46 (dd, J=12.3, 2.2 Hz, 1H), 8.03 (dd, J=16.7, 8.0 Hz, 1H), 7.77 (ddd, J=41.9, 8.0, 2.2 Hz, 1H), 7.69-7.56 (m, 2H), 7.11 (d, J=1.6 Hz, 1H), 7.03 (dd, J=8.4, 5.9 Hz, 2H), 6.94-6.82 (m, 2H), 6.45 (t, J=2.4 Hz, 1H), 4.84 (d, J=15.9 Hz, 2H), 4.57 (s, 1H), 4.35 (s, 1H), 4.26 (s, 1H), 4.12 (s, 1H), 3.82 (t, J=5.8 Hz, 1H), 3.67-3.53 (m, 3H), 3.15-3.10 (m, 3H), 3.05 (t, J=6.5 Hz, 1H), 2.86 (t, J=5.6 Hz, 1H), 2.78 (t, J=5.8 Hz, 1H), 2.48-2.38 (m, 2H), 2.29-2.19 (m, 2H), 1.61-1.54 (m, 2H), 0.54-0.48 (m, 3H). MS (ESI) m/z 867.1 [M+H]⁺.

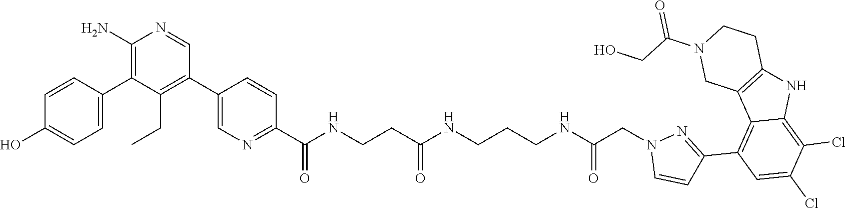 [SMILES: CCc1c(-c2ccc(C(=O)NCCC(=O)NCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 114: 6′-amino-N-(3-((4-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)butyl)-amino)-3-oxopropyl)-4′-ethyl-5′-(4-hydroxyphenyl)-[3,3′-bipyridine]-6-carboxamide (KL186-131)

Example 114 was synthesized following similar procedure for preparing example 112 from intermediate 7 and linker 59. Light yellow solid, 5.2 mg, 59% yield. ¹H NMR (400 MHz, Methanol-d₄) δ 8.48 (t, J=2.8 Hz, 1H), 8.04 (dd, J=8.1, 5.7 Hz, 1H), 7.84-7.79 (m, 1H), 7.68-7.57 (m, 2H), 7.09 (d, J=2.4 Hz, 1H), 7.03 (dd, J=8.7, 7.1 Hz, 2H), 6.89 (d, J=8.2 Hz, 2H), 6.45 (dd, J=5.4, 2.3 Hz, 1H), 4.83 (d, J=11.5 Hz, 2H), 4.57 (s, 1H), 4.34 (s, 1H), 4.26 (s, 1H), 4.12 (s, 1H), 3.83 (t, J=5.8 Hz, 1H), 3.65-3.58 (m, 3H), 3.10 (t, J=6.4, 3H), 3.05-2.97 (m, 1H), 2.86 (t, J=5.8 Hz, 1H), 2.78 (t, J=5.9 Hz, 1H), 2.42 (q, J=6.5 Hz, 2H), 2.32-2.16 (m, 2H), 1.42-1.36 (m, 4H), 0.67-0.45 (m, 3H). MS (ESI) m/z 881.3 [M+H]⁺.

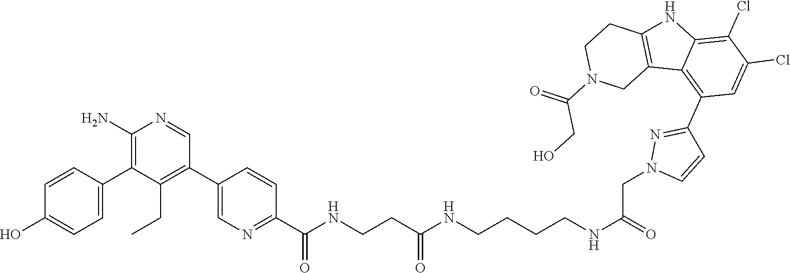 [SMILES: CCc1c(-c2ccc(C(=O)NCCC(=O)NCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 115: 6′-amino-N-(3-((5-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)pentyl)-amino)-3-oxopropyl)-4′-ethyl-5′-(4-hydroxyphenyl)-[3,3′-bipyridine]-6-carboxamide (KL186-132)

Example 115 was synthesized following similar procedure for preparing example 112 from intermediate 7 and linker 60. Light yellow solid, 5.0 mg, 56% yield. ¹H NMR (400 MHz, Methanol-d₄) δ 8.47 (t, J=2.8 Hz, 1H), 8.05 (t, J=7.7 Hz, 1H), 7.84-7.78 (m, 1H), 7.69-7.54 (m, 2H), 7.10 (d, J=2.4 Hz, 1H), 7.06-7.00 (m, 2H), 6.89 (d, J=8.4 Hz, 2H), 6.45 (t, J=2.8 Hz, 1H), 4.84 (d, J=9.4 Hz, 2H), 4.57 (s, 1H), 4.34 (s, 1H), 4.24 (s, 1H), 4.12 (s, 1H), 3.82 (t, J=5.9 Hz, 1H), 3.63-3.58 (m, 3H), 3.11-3.00 (m, 4H), 2.86 (t, J=5.8 Hz, 1H), 2.78 (t, J=5.9 Hz, 1H), 2.45-2.39 (m, 2H), 2.30-2.22 (m, 2H), 1.42-1.31 (m, 4H), 1.28-1.09 (m, 2H), 0.53 (q, J=7.8 Hz, 3H). MS (ESI) m/z 894.4 [M+H]⁺.

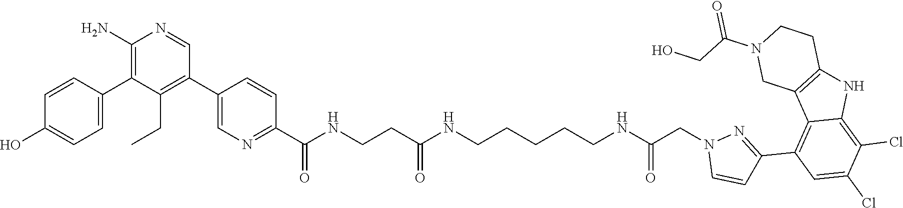 [SMILES: CCc1c(-c2ccc(C(=O)NCCC(=O)NCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 116: 6′-amino-N-(3-((6-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)hexyl)-amino)-3-oxopropyl)-4′-ethyl-5′-(4-hydroxyphenyl)-[3,3′-bipyridine]-6-carboxamide (KL186-133)

Example 116 was synthesized following similar procedure for preparing example 112 from intermediate 7 and linker 61. Light yellow solid, 5.5 mg, 60% yield. ¹H NMR (400 MHz, Methanol-d₄) δ 8.46 (d, J=2.1 Hz, 1H), 8.05 (t, J=7.8 Hz, 1H), 7.88-7.76 (m, 1H), 7.72-7.63 (m, 1H), 7.60 (d, J=6.2 Hz, 1H), 7.11 (s, 1H), 7.08-6.98 (m, 2H), 6.90 (dd, J=8.6, 2.0 Hz, 2H), 6.46 (d, J=2.4 Hz, 1H), 4.84 (d, J=6.2 Hz, 2H), 4.59 (s, 1H), 4.35 (s, 1H), 4.24 (s, 1H), 4.12 (s, 1H), 3.84 (t, J=5.8 Hz, 1H), 3.62 (d, J=7.0 Hz, 3H), 3.12-3.00 (m, 4H), 2.88 (d, J=8.9 Hz, 1H), 2.81 (d, J=7.0 Hz, 1H), 2.45-2.39 (m, 2H), 2.31-2.25 (m, 2H), 1.43-1.25 (m, 4H), 1.25-1.07 (m, 4H), 0.57-0.51 (m, 3H). MS (ESI) m/z 909.3 [M+H]⁻.

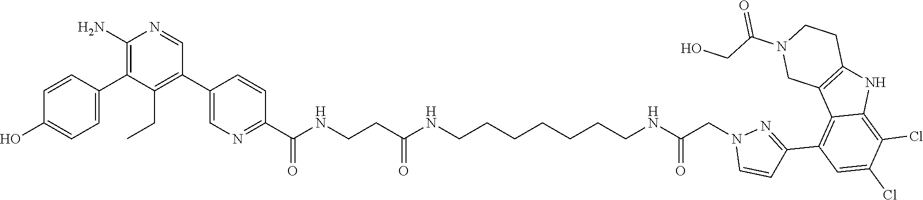 [SMILES: CCc1c(-c2ccc(C(=O)NCCC(=O)NCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 117: 6′-amino-N-(3-((7-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)heptyl)amino)-3-oxopropyl)-4′-ethyl-5′-(4-hydroxyphenyl)-[3,3′-bipyridine]-6-carboxamide (KL186-134)

Example 117 was synthesized following similar procedure for preparing example 112 from intermediate 7 and linker 62. Light yellow solid, 4.8 mg, 52% yield. ¹H NMR (400 MHz, Methanol-d₄) δ 8.47 (d, J=2.4 Hz, 1H), 8.06 (dd, J=8.1, 4.2 Hz, 1H), 7.87-7.80 (m, 1H), 7.66 (d, J=2.3 Hz, 1H), 7.60 (d, J=5.2 Hz, 1H), 7.11 (d, J=1.7 Hz, 1H), 7.09-6.97 (m, 2H), 6.90 (dd, J=8.5, 2.1 Hz, 2H), 6.46 (t, J=2.1 Hz, 1H), 4.89-4.81 (m, 2H), 4.56 (d, J=15.0 Hz, 1H), 4.36 (s, 1H), 4.24 (s, 1H), 4.12 (s, 1H), 3.84 (t, J=5.9 Hz, 1H), 3.62 (q, J=6.2 Hz, 3H), 3.13-3.02 (m, 4H), 2.87 (s, 1H), 2.80 (t, J=5.8 Hz, 1H), 2.43 (q, J=8.0, 7.3 Hz, 2H), 2.29 (q, J=7.2 Hz, 2H), 1.46-1.25 (m, 4H), 1.27-1.00 (m, 6H), 0.67-0.43 (m, 3H). MS (ESI) m/z 923.4 [M+H]⁺.

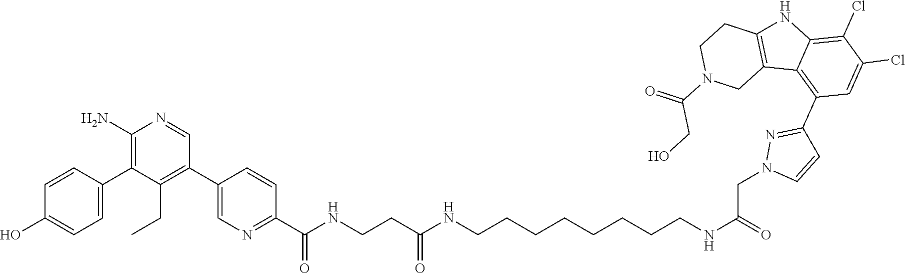 [SMILES: CCc1c(-c2ccc(C(=O)NCCC(=O)NCCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 118: 6′-amino-N-(3-((8-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)octyl)amino)-3-oxopropyl)-4′-ethyl-5′-(4-hydroxyphenyl)-[3,3′-bipyridine]-6-carboxamide (KL186-135)

Example 118 was synthesized following similar procedure for preparing example 112 from intermediate 7 and linker 63. Light yellow solid, 6.3 mg, 67% yield. ¹H NMR (400 MHz, Methanol-d₄) δ 8.44 (dd, J=20.9, 2.1 Hz, 1H), 8.06 (t, J=8.0 Hz, 1H), 7.80 (ddd, J=30.6, 8.0, 2.2 Hz, 1H), 7.69-7.52 (m, 2H), 7.11 (s, 1H), 7.09-6.99 (m, 2H), 6.90 (dd, J=8.6, 2.5 Hz, 2H), 6.46 (d, J=2.3 Hz, 1H), 4.85 (s, 2H), 4.60 (s, 1H), 4.35 (s, 1H), 4.22 (s, 1H), 4.12 (s, 1H), 3.84 (t, J=5.8 Hz, 1H), 3.61 (t, J=6.4 Hz, 3H), 3.11 (q, J=6.9 Hz, 2H), 3.05 (q, J=6.7 Hz, 2H), 2.87 (d, J=6.0 Hz, 1H), 2.80 (t, J=5.9 Hz, 1H), 2.43 (q, J=5.9 Hz, 2H), 2.36-2.16 (m, 2H), 1.45-1.24 (m, 4H), 1.24-0.99 (m, 8H), 0.59-0.52 (m, 3H). MS (ESI) m/z 937.4 [M+H]⁺.

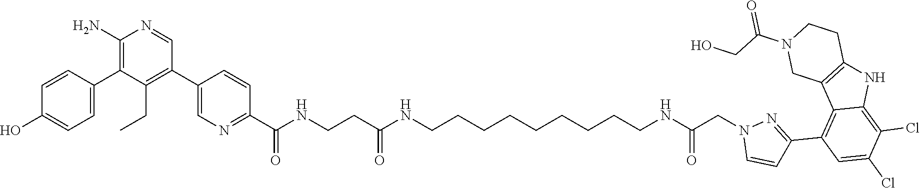 [SMILES: CCc1c(-c2ccc(C(=O)NCCC(=O)NCCCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 119: 6′-amino-N-(3-((9-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)nonyl)amino)-3-oxopropyl)-4′-ethyl-5′-(4-hydroxyphenyl)-[3,3′-bipyridine]-6-carboxamide (KL186-136)

Example 119 was synthesized following similar procedure for preparing example 112 from intermediate 7 and linker 64. Light yellow solid, 5.5 mg, 58% yield. ¹H NMR (400 MHz, Methanol-d₄) δ 8.59 (dd, J=12.5, 2.2 Hz, 1H), 8.18 (dd, J=8.1, 5.1 Hz, 1H), 7.95 (ddd, J=21.2, 8.0, 2.2 Hz, 1H), 7.82-7.64 (m, 2H), 7.23 (d, J=1.8 Hz, 1H), 7.19-7.12 (m, 2H), 7.02 (dd, J=8.6, 2.8 Hz, 2H), 6.57 (t, J=2.6 Hz, 1H), 4.97 (s, 2H), 4.70 (s, 1H), 4.47 (s, 1H), 4.34 (s, 1H), 4.24 (s, 1H), 3.95 (t, J=5.9 Hz, 1H), 3.73 (t, J=6.4 Hz, 3H), 3.28-3.13 (m, 4H), 2.98 (t, J=5.9 Hz, 1H), 2.92 (t, J=6.0 Hz, 1H), 2.57-2.53 (m, 2H), 2.44-2.36 (m, 2H), 1.54-1.42 (m, 4H), 1.38-1.03 (m, 10H), 0.68 (q, J=7.9 Hz, 3H). MS (ESI) m/z 951.4 [M+H]⁺.

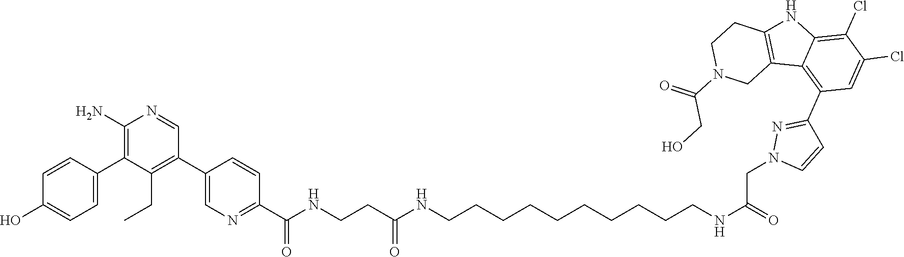 [SMILES: CCc1c(-c2ccc(C(=O)NCCC(=O)NCCCCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 120: 6′-amino-N-(3-((10-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)decyl)amino)-3-oxopropyl)-4′-ethyl-5′-(4-hydroxyphenyl)-[3,3′-bipyridine]-6-carboxamide (KL186-137)

Example 120 was synthesized following similar procedure for preparing example 112 from intermediate 7 and linker 65. Light yellow solid, 5.7 mg, 59% yield. ¹H NMR (400 MHz, Methanol-d₄) δ 8.48 (dd, J=15.8, 2.2 Hz, 1H), 8.07 (dd, J=8.0, 5.8 Hz, 1H), 7.84 (ddd, J=22.9, 8.0, 2.2 Hz, 1H), 7.69-7.50 (m, 2H), 7.11 (s, 1H), 7.07-6.98 (m, 2H), 6.89 (dd, J=8.6, 3.0 Hz, 2H), 6.45 (t, J=2.7 Hz, 1H), 4.85 (s, 2H), 4.58 (s, 1H), 4.34 (s, 1H), 4.22 (s, 1H), 4.12 (s, 1H), 3.83 (t, J=5.8 Hz, 1H), 3.61 (t, J=6.4 Hz, 3H), 3.16-3.00 (m, 4H), 2.86 (t, J=5.7 Hz, 1H), 2.83-2.75 (m, 1H), 2.43 (t, J=6.4 Hz, 2H), 2.35-2.23 (m, 2H), 1.41-1.32 (m, 4H), 1.18-1.05 (m, 12H), 0.64-0.50 (m, 3H). MS (ESI) m/z 965.4 [M+H]⁺.

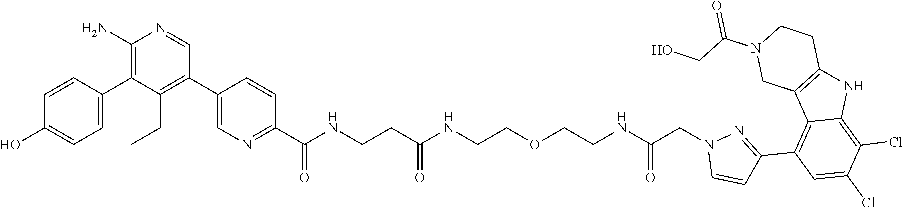 [SMILES: CCc1c(-c2ccc(C(=O)NCCC(=O)NCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 121: 6′-amino-N-(3-((2-(2-(2-(3-(6,7-dichloro-2-(2-hydroxyacetyl)-2,3,4,5-tetrahydro-1H-pyrido[4,3-b]indol-9-yl)-1H-pyrazol-1-yl)acetamido)ethoxy)-ethyl)amino)-3-oxopropyl)-4′-ethyl-5′-(4-hydroxyphenyl)-[3,3′-bipyridine]-6-carboxamide (KL186-138)

Example 121 was synthesized following similar procedure for preparing example 112 from intermediate 7 and linker 66. Light yellow solid, 5.7 mg, 64% yield. ¹H NMR (400 MHz, Methanol-d₄) δ 8.47 (t, J=3.2 Hz, 1H), 8.03 (d, J=8.0 Hz, 1H), 7.79 (dt, J=8.3, 2.5 Hz, 1H), 7.67 (t, J=2.0 Hz, 1H), 7.62 (s, 1H), 7.10 (d, J=1.8 Hz, 1H), 7.07-7.01 (m, 2H), 6.90 (d, J=8.3 Hz, 2H), 6.45 (dd, J=5.7, 2.3 Hz, 1H), 4.89 (d, J=17.6 Hz, 2H), 4.56 (s, 1H), 4.37 (s, 1H), 4.25 (s, 1H), 4.13 (s, 1H), 3.83 (t, J=5.8 Hz, 1H), 3.69-3.51 (m, 3H), 3.45-3.33 (m, 4H), 3.26 (t, J=6.0 Hz, 4H), 2.86 (s, 1H), 2.80 (q, J=5.9, 4.5 Hz, 1H), 2.46-2.40 (m, 2H), 2.32-2.17 (m, 2H), 0.55-0.50 (m, 3H). MS (ESI) m/z 897.4 [M+H]⁺.

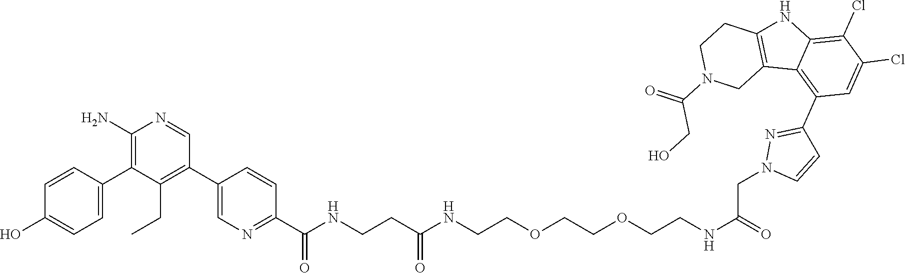 [SMILES: CCc1c(-c2ccc(C(=O)NCCC(=O)NCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 122: 6′-amino-N-(1-(3-(6,7-dichloro-2-(2-hydroxyacetyl)-2,3,4,5-tetrahydro-1H-pyrido[4,3-b]indol-9-yl)-1H-pyrazol-1-yl)-2,13-dioxo-6,9-dioxa-3,12-diazapentadecan-15-yl)-4′-ethyl-5′-(4-hydroxyphenyl)-[3,3′-bipyridine]-6-carboxamide (KL186-139)

Example 122 was synthesized following similar procedure for preparing example 112 from intermediate 7 and linker 67. Light yellow solid, 5.4 mg, 57% yield. ¹H NMR (400 MHz, Methanol-d₄) δ 8.46 (t, J=3.4 Hz, 1H), 8.04 (d, J=8.0 Hz, 1H), 7.82-7.78 (m, 1H), 7.67 (d, J=2.3 Hz, 1H), 7.61 (d, J=2.8 Hz, 1H), 7.11 (s, 1H), 7.09-7.00 (m, 2H), 6.95-6.83 (m, 2H), 6.46 (dd, J=5.1, 2.3 Hz, 1H), 4.88 (d, J=11.7 Hz, 2H), 4.58 (s, 1H), 4.39 (s, 1H), 4.24 (s, 1H), 4.13 (s, 1H), 3.84 (t, J=5.7 Hz, 1H), 3.64-3.59 (m, 3H), 3.46-3.38 (m, 6H), 3.38-3.23 (m, 6H), 2.87 (s, 1H), 2.80 (t, J=6.0 Hz, 1H), 2.51-2.38 (m, 2H), 2.26 (q, J=7.6 Hz, 2H), 0.54 (t, J=7.5 Hz, 3H). MS (ESI) m/z 941.3 [M+H]⁺.

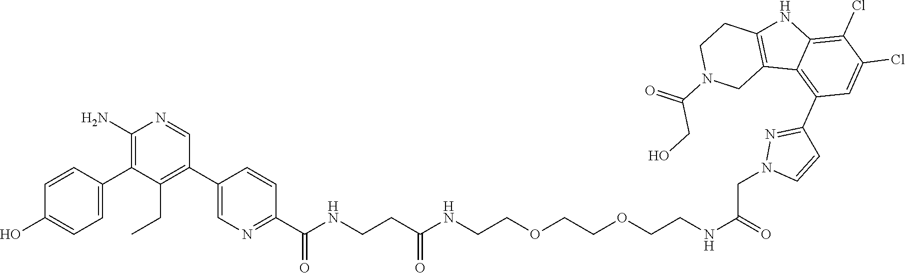 [SMILES: CCc1c(-c2ccc(C(=O)NCCC(=O)NCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 123: 6′-amino-N-(1-(3-(6,7-dichloro-2-(2-hydroxyacetyl)-2,3,4,5-tetrahydro-1H-pyrido[4,3-b]indol-9-yl)-1H-pyrazol-1-yl)-2,16-dioxo-6,9,12-trioxa-3,15-diazaoctadecan-18-yl)-4′-ethyl-5′-(4-hydroxyphenyl)-[3,3′-bipyridine]-6-carboxamide (KL186-140)

Example 123 was synthesized following similar procedure for preparing example 112 from intermediate 7 and linker 68. Light yellow solid, 6.1 mg, 62% yield. ¹H NMR (400 MHz, Methanol-d₄) δ 8.51-8.44 (m, 1H), 8.05 (t, J=7.4 Hz, 1H), 7.82 (ddd, J=17.0, 8.1, 2.2 Hz, 1H), 7.67 (d, J=2.3 Hz, 1H), 7.63 (s, 1H), 7.12 (s, 1H), 7.04 (t, J=8.2 Hz, 2H), 6.90 (dd, J=8.6, 1.8 Hz, 2H), 6.46 (dd, J=5.4, 2.3 Hz, 1H), 4.88 (d, J=9.1 Hz, 2H), 4.58 (s, 1H), 4.38 (s, 1H), 4.23 (s, 1H), 4.13 (s, 1H), 3.84 (t, J=5.8 Hz, 1H), 3.64-3.55 (m, 3H), 3.48-3.37 (m, 12H), 3.35-3.24 (m, 4H), 2.88 (d, J=12.0 Hz, 1H), 2.86-2.78 (m, 1H), 2.44 (t, J=6.5 Hz, 2H), 2.28 (q, J=6.9 Hz, 2H), 0.61-0.53 (m, 3H). MS (ESI) m/z 985.4 [M+H]⁺.

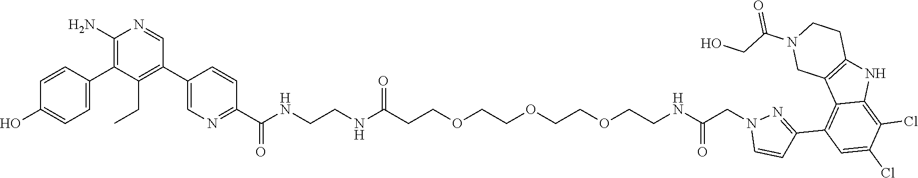 [SMILES: CCc1c(-c2ccc(C(=O)NCCNC(=O)CCOCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 124: 6′-amino-N-(1-(3-(6,7-dichloro-2-(2-hydroxyacetyl)-2,3,4,5-tetrahydro-1H-pyrido[4,3-b]indol-9-yl)-1H-pyrazol-1-yl)-2,19-dioxo-6,9,12,15-tetraoxa-3,18-diazahenicosan-21-yl)-4′-ethyl-5′-(4-hydroxyphenyl)-[3,3′-bipyridine]-6-carboxamide (KL186-141)

Example 124 was synthesized following similar procedure for preparing example 112 from intermediate 7 and linker 69. Light yellow solid, 6.0 mg, 58% yield. ¹H NMR (400 MHz, Methanol-d₄) δ 8.48 (dd, J=15.2, 2.1 Hz, 1H), 8.06 (t, J=7.8 Hz, 1H), 7.83 (ddd, J=18.5, 8.0, 2.2 Hz, 1H), 7.66 (dd, J=9.8, 2.9 Hz, 2H), 7.12 (s, 1H), 7.09-6.99 (m, 2H), 6.90 (dd, J=8.6, 2.1 Hz, 2H), 6.46 (dd, J=5.3, 2.3 Hz, 1H), 4.88 (d, J=8.1 Hz, 2H), 4.58 (s, 1H), 4.38 (s, 1H), 4.23 (s, 1H), 4.12 (s, 1H), 3.84 (t, J=5.9 Hz, 1H), 3.64-3.58 (m, 3H), 3.52-3.38 (m, 14H), 3.37-3.28 (m, 2H), 3.28-3.22 (m, 4H), 2.86 (s, 1H), 2.80 (t, J=5.8 Hz, 1H), 2.45 (t, J=6.5 Hz, 2H), 2.29 (q, J=7.6 Hz, 2H), 0.56 (q, J=7.3 Hz, 3H). MS (ESI) m/z 1029.4 [M+H]⁺.

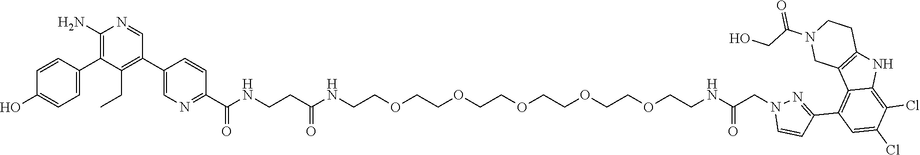 [SMILES: CCc1c(-c2ccc(C(=O)NCCC(=O)NCCOCCOCCOCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1]

### Example 125: 6′-amino-N-(1-(3-(6,7-dichloro-2-(2-hydroxyacetyl)-2,3,4,5-tetrahydro-1H-pyrido[4,3-b]indol-9-yl)-1H-pyrazol-1-yl)-2,22-dioxo-6,9,12,15,18-pentaoxa-3,21-diazatetracosan-24-yl)-4′-ethyl-5′-(4-hydroxyphenyl)-[3,3′-bipyridine]-6-carboxamide (KL186-142)

Example 125 was synthesized following similar procedure for preparing example 112 from intermediate 7 and linker 70. Light yellow solid, 6.6 mg, 61% yield. ¹H NMR (400 MHz, Methanol-d₄) δ 8.49 (dd, J=16.5, 2.1 Hz, 1H), 8.06 (t, J=7.4 Hz, 1H), 7.85 (ddd, J=19.0, 8.0, 2.2 Hz, 1H), 7.72-7.59 (m, 2H), 7.13 (s, 1H), 7.11-7.00 (m, 2H), 6.90 (dd, J=8.7, 2.4 Hz, 2H), 6.46 (dd, J=5.0, 2.3 Hz, 1H), 4.88 (d, J=7.8 Hz, 2H), 4.58 (s, 1H), 4.38 (s, 1H), 4.23 (s, 1H), 4.12 (s, 1H), 3.85 (t, J=5.9 Hz, 1H), 3.65-3.59 (m, 3H), 3.55-3.37 (m, 18H), 3.35-3.22 (m, 6H), 2.87 (s, 1H), 2.80 (t, J=5.8 Hz, 1H), 2.45 (t, J=6.5 Hz, 2H), 2.30 (q, J=7.6 Hz, 2H), 0.57 (q, J=7.3 Hz, 3H). MS (ESI) m/z 1073.4 [M+H]⁺.

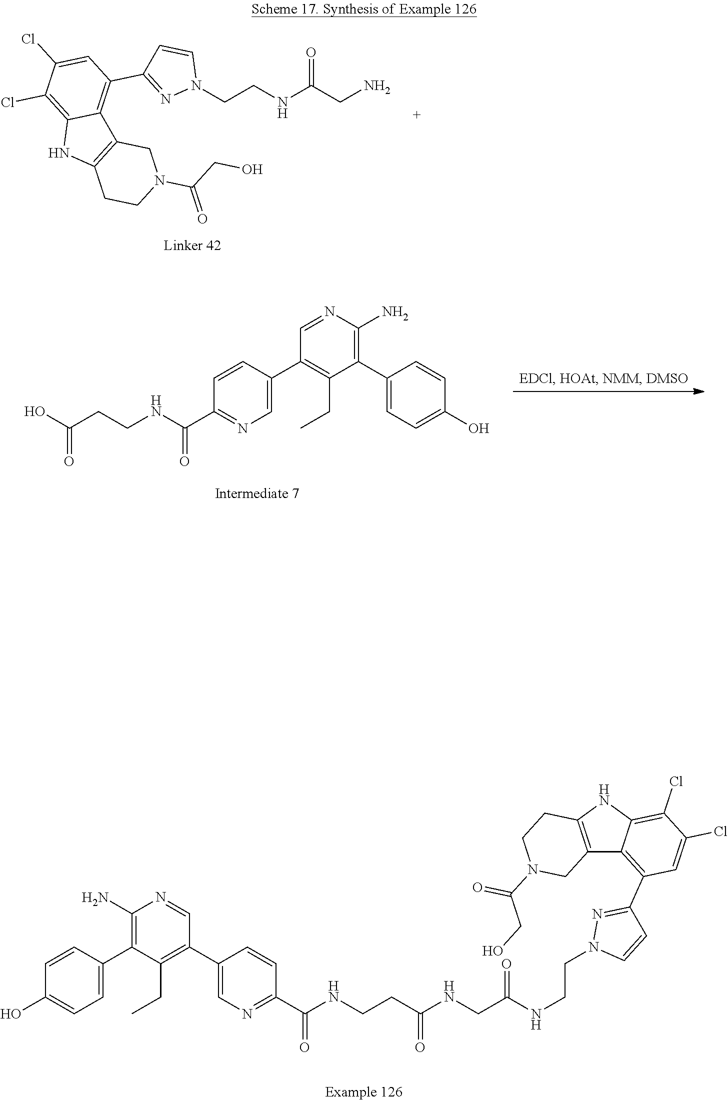 [SMILES: NCC(=O)NCCn1ccc(-c2cc(Cl)c(Cl)c3[nH]c4c(c23)CN(C(=O)CO)CC4)n1 ; CCc1c(-c2ccc(C(=O)NCCC(=O)O)nc2)cnc(N)c1-c1ccc(O)cc1 ; CCc1c(-c2ccc(C(=O)NCCC(=O)NCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 126: 6′-amino-N-(3-((2-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-2-oxoethyl)amino)-3-oxopropyl)-4′-ethyl-5′-(4-hydroxyphenyl)-[3,3′-bipyridine]-6-carboxamide (KL186-144)

To a solution of intermediate 7 (4 mg, 0.01 mmol) in DMSO (1 mL) were added linker 42 (4.6 mg, 0.01 mmol, 1.0 equiv), EDCI (1-ethyl-3-(3-dimethylaminopropyl)carbodiimide) (3.0 mg, 0.015 mmol, 1.5 equiv), HOAt (1-hydroxy-7-azabenzo-triazole) (2.0 mg, 0.015 mmol, 1.5 equiv), and NMM (N-Methylmorpholine) (3.0 mg, 0.03 mmol, 3.0 equiv). After being stirred overnight at room temperature, the resulting mixture was purified by preparative HPLC to afford titled compound as light yellow solid (4.7 mg, 55%). ¹H NMR (400 MHz, Methanol-d₄) δ 8.50 (dd, J=9.4, 2.0 Hz, 1H), 8.05 (dd, J=24.4, 8.1 Hz, 1H), 7.86-7.72 (m, 1H), 7.68-7.62 (m, 1H), 7.57 (d, J=2.3 Hz, 1H), 7.17-7.00 (m, 3H), 6.97-6.84 (m, 2H), 6.36 (dd, J=41.2, 2.3 Hz, 1H), 4.45 (d, J=12.1 Hz, 2H), 4.35-4.23 (m, 3H), 4.13 (s, 1H), 3.84 (t, J=5.7 Hz, 1H), 3.75 (d, J=11.2 Hz, 2H), 3.65-3.56 (m, 5H), 2.88 (t, J=5.8 Hz, 1H), 2.81 (d, J=10.5 Hz, 1H), 2.46 (t, J=6.3 Hz, 2H), 2.27 (q, J=7.5 Hz, 2H), 0.58-0.53 (m, 3H). MS (ESI) m/z 853.3 [M+H]⁺.

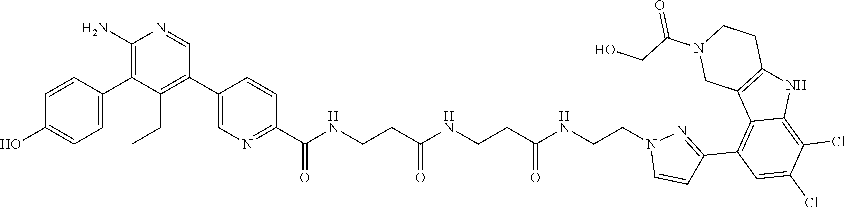 [SMILES: CCc1c(-c2ccc(C(=O)NCCC(=O)NCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 127: 6′-amino-N-(3-((3-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-3-oxopropyl)amino)-3-oxopropyl)-4′-ethyl-5′-(4-hydroxyphenyl)-[3,3′-bipyridine]-6-carboxamide (KL186-145)

Example 127 was synthesized following similar procedure for preparing example 126 from intermediate 7 and linker 43. Light yellow solid, 5.0 mg, 58% yield. ¹H NMR (400 MHz, Methanol-d₄) δ 8.47 (dd, J=18.4, 2.1 Hz, 1H), 8.03 (dd, J=10.0, 7.9 Hz, 1H), 7.81 (ddd, J=29.7, 8.1, 2.2 Hz, 1H), 7.71-7.50 (m, 2H), 7.14-6.99 (m, 3H), 6.95-6.83 (m, 2H), 6.37 (dd, J=37.6, 2.3 Hz, 1H), 4.42 (d, J=20.0 Hz, 2H), 4.30-4.21 (m, 3H), 4.12 (s, 1H), 3.83 (t, J=5.8 Hz, 1H), 3.62 (t, J=5.7 Hz, 1H), 3.59-3.48 (m, 4H), 3.31 (q, J=6.9 Hz, 2H), 2.87 (t, J=5.7 Hz, 1H), 2.80 (q, J=6.1, 4.3 Hz, 1H), 2.38 (t, J=6.4 Hz, 2H), 2.34-2.16 (m, 4H), 0.66-0.42 (m, 3H). MS (ESI) m/z 867.4 [M+H]⁺.

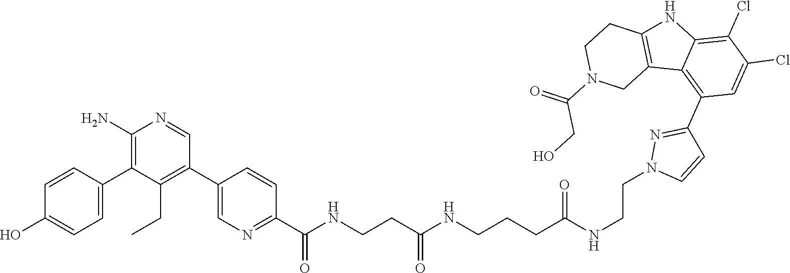 [SMILES: CCc1c(-c2ccc(C(=O)NCCC(=O)NCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 128: 6′-amino-N-(3-((4-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-4-oxobutyl)amino)-3-oxopropyl)-4′-ethyl-5′-(4-hydroxyphenyl)-[3,3′-bipyridine]-6-carboxamide (KL186-146)

Example 128 was synthesized following similar procedure for preparing example 126 from intermediate 7 and linker 44. Light yellow solid, 5.5 mg, 62% yield. ¹H NMR (400 MHz, Methanol-d₄) δ 8.48 (dd, J=12.5, 2.2 Hz, 1H), 8.05 (dd, J=11.5, 8.0 Hz, 1H), 7.83 (ddd, J=22.4, 8.0, 2.2 Hz, 1H), 7.73-7.48 (m, 2H), 7.16-7.01 (m, 3H), 6.97-6.86 (m, 2H), 6.38 (dd, J=33.9, 2.3 Hz, 1H), 4.46 (d, J=7.1 Hz, 2H), 4.28 (q, J=4.7, 4.2 Hz, 3H), 4.13 (s, 1H), 3.84 (t, J=5.8 Hz, 1H), 3.66-3.58 (m, 5H), 3.09-3.04 (m, 2H), 2.85 (dt, J=29.0, 5.9 Hz, 2H), 2.41 (t, J=6.4 Hz, 2H), 2.27 (q, J=7.4 Hz, 2H), 2.10-2.05 (m, 2H), 1.67-1.60 (m, 2H), 0.67-0.47 (m, 3H). MS (ESI) m/z 881.3 [M+H]⁺.

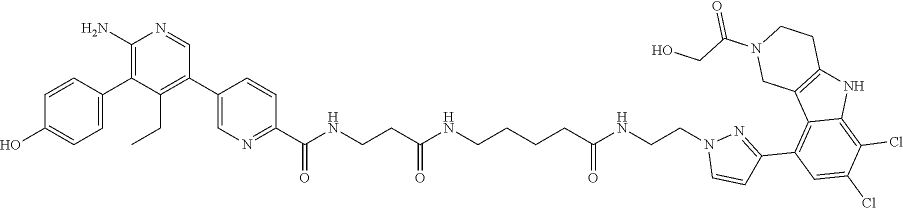 [SMILES: CCc1c(-c2ccc(C(=O)NCCC(=O)NCCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 129: 6′-amino-N-(3-((5-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-5-oxopentyl)amino)-3-oxopropyl)-4′-ethyl-5′-(4-hydroxyphenyl)-[3,3′-bipyridine]-6-carboxamide (KL186-147)

Example 129 was synthesized following similar procedure for preparing example 126 from intermediate 7 and linker 45. Light yellow solid, 5.1 mg, 57% yield. ¹H NMR (400 MHz, Methanol-d₄) δ 8.50 (t, J=3.0 Hz, 1H), 8.06 (dd, J=8.0, 2.3 Hz, 1H), 7.86 (ddd, J=8.0, 5.7, 2.2 Hz, 1H), 7.75-7.46 (m, 2H), 7.19-6.98 (m, 3H), 6.96-6.82 (m, 2H), 6.38 (dd, J=27.0, 2.3 Hz, 1H), 4.47 (d, J=11.6 Hz, 2H), 4.26 (d, J=3.3 Hz, 3H), 4.12 (s, 1H), 3.84 (t, J=5.9 Hz, 1H), 3.64 (t, J=5.7 Hz, 1H), 3.61-3.49 (m, 4H), 3.03 (t, J=6.7 Hz, 2H), 2.87 (t, J=5.8 Hz, 1H), 2.80 (t, J=5.7 Hz, 1H), 2.50-2.33 (m, 2H), 2.29 (q, J=7.5 Hz, 2H), 2.08-2.03 (m, 2H), 1.55-1.39 (m, 2H), 1.41-1.20 (m, 2H), 0.57 (t, J=7.5 Hz, 3H). MS (ESI) m/z 895.4 [M+H]⁻.

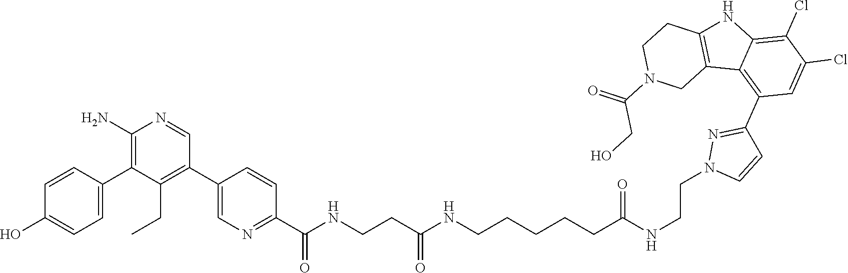 [SMILES: CCc1c(-c2ccc(C(=O)NCCC(=O)NCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 130: 6′-amino-N-(3-((6-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-6-oxohexyl)amino)-3-oxopropyl)-4′-ethyl-5′-(4-hydroxyphenyl)-[3,3′-bipyridine]-6-carboxamide (KL186-148)

Example 130 was synthesized following similar procedure for preparing example 126 from intermediate 7 and linker 46. Light yellow solid, 4.9 mg, 54% yield. ¹H NMR (400 MHz, Methanol-d₄) δ 8.51 (dd, J=8.1, 2.2 Hz, 1H), 8.07 (dd, J=8.1, 3.9 Hz, 1H), 7.87 (ddd, J=10.3, 8.1, 2.2 Hz, 1H), 7.74-7.48 (m, 2H), 7.18-7.01 (m, 3H), 6.96-6.83 (m, 2H), 6.39 (dd, J=28.6, 2.3 Hz, 1H), 4.48 (d, J=12.4 Hz, 2H), 4.26 (d, J=5.1 Hz, 3H), 4.14 (s, 1H), 3.85 (t, J=5.8 Hz, 1H), 3.71-3.48 (m, 5H), 3.03 (t, J=6.9 Hz, 2H), 2.88 (t, J=5.8 Hz, 1H), 2.85-2.69 (m, 1H), 2.44-2.40 (m, 2H), 2.33-2.27 (m, 2H), 2.02 (t, J=7.5 Hz, 2H), 1.45-1.37 (m, 2H), 1.35-1.28 (m, 2H), 1.20-1.11 (m, 2H), 0.60-0.56 (m, 3H). MS (ESI) m/z 909.3 [M+H]⁺.

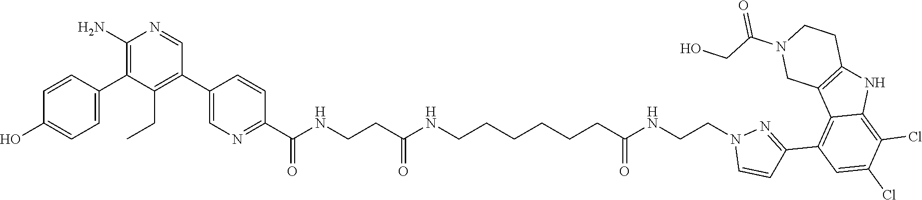 [SMILES: CCc1c(-c2ccc(C(=O)NCCC(=O)NCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 131: 6′-amino-N-(3-((7-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-7-oxoheptyl)amino)-3-oxopropyl)-4′-ethyl-5′-(4-hydroxyphenyl)-[3,3′-bipyridine]-6-carboxamide (KL186-149)

Example 131 was synthesized following similar procedure for preparing example 126 from intermediate 7 and linker 47. Light yellow solid, 5.4 mg, 58% yield. ¹H NMR (400 MHz, Methanol-d₄) δ 8.52 (d, J=2.2 Hz, 1H), 8.07 (d, J=8.0 Hz, 1H), 7.87 (dt, J=8.2, 2.6 Hz, 1H), 7.74-7.46 (m, 2H), 7.19-7.00 (m, 3H), 6.96-6.79 (m, 2H), 6.39 (dd, J=27.1, 2.3 Hz, 1H), 4.48 (d, J=16.3 Hz, 2H), 4.35-4.20 (m, 3H), 4.13 (s, 1H), 3.84 (t, J=5.9 Hz, 1H), 3.65-3.55 (m, 5H), 3.04-3.00 (m, 2H), 2.87 (t, J=5.8 Hz, 1H), 2.81 (t, J=5.6 Hz, 1H), 2.41 (q, J=7.0 Hz, 2H), 2.30 (q, J=7.5 Hz, 2H), 2.13-1.97 (m, 2H), 1.35 (dt, J=34.2, 7.4 Hz, 4H), 1.16-1.10 (m, 4H), 0.58 (t, J=7.5 Hz, 3H). MS (ESI) m/z 923.3 [M+H]⁺.

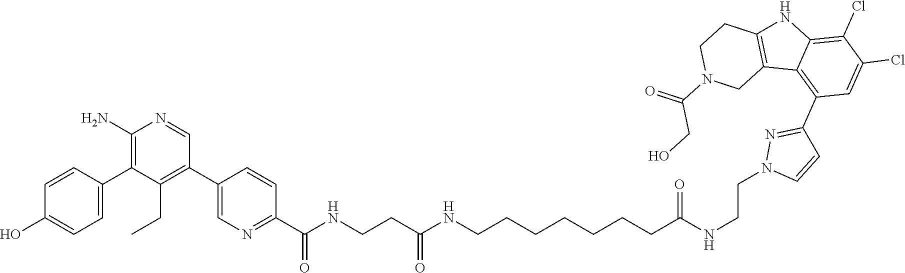 [SMILES: CCc1c(-c2ccc(C(=O)NCCC(=O)NCCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 132: 6′-amino-N-(3-((8-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-8-oxooctyl)amino)-3-oxopropyl)-4′-ethyl-5′-(4-hydroxyphenyl)-[3,3′-bipyridine]-6-carboxamide (KL186-150)

Example 132 was synthesized following similar procedure for preparing example 126 from intermediate 7 and linker 48. Light yellow solid, 6.1 mg, 65% yield. ¹H NMR (400 MHz, Methanol-d₄) δ 8.52 (d, J=2.6 Hz, 1H), 8.08 (d, J=8.0 Hz, 1H), 7.91-7.87 (m, 1H), 7.73-7.47 (m, 2H), 7.17-7.01 (m, 3H), 6.96-6.82 (m, 2H), 6.39 (dd, J=26.7, 2.2 Hz, 1H), 4.48 (d, J=16.1 Hz, 2H), 4.27 (dd, J=11.7, 6.3 Hz, 3H), 4.14 (s, 1H), 3.85 (t, J=5.7 Hz, 1H), 3.65-3.55 (m, 5H), 3.02 (q, J=6.9 Hz, 2H), 2.84 (dt, J=25.3, 5.6 Hz, 2H), 2.42 (q, J=6.0 Hz, 2H), 2.30 (q, J=7.5 Hz, 2H), 2.03 (q, J=8.7, 8.2 Hz, 2H), 1.47-1.26 (m, 4H), 1.10 (d, J=8.9 Hz, 6H), 0.58 (t, J=7.5 Hz, 3H). MS (ESI) m/z 937.4 [M+H]⁺.

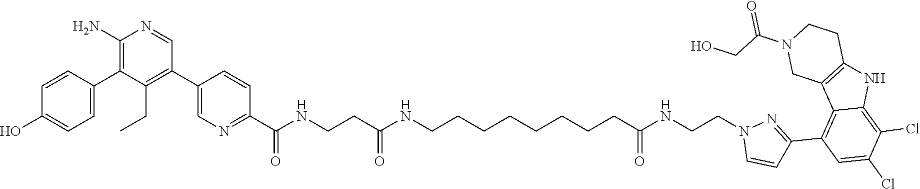 [SMILES: CCc1c(-c2ccc(C(=O)NCCC(=O)NCCCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 133: 6′-amino-N-(3-((9-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-9-oxononyl)amino)-3-oxopropyl)-4′-ethyl-5′-(4-hydroxyphenyl)-[3,3′-bipyridine]-6-carboxamide (KL186-151)

Example 133 was synthesized following similar procedure for preparing example 126 from intermediate 7 and linker 49. Light yellow solid, 5.0 mg, 53% yield. ¹H NMR (400 MHz, Methanol-d₄) δ 8.52 (d, J=2.4 Hz, 1H), 8.08 (d, J=8.0 Hz, 1H), 7.89 (ddd, J=7.9, 4.6, 2.2 Hz, 1H), 7.74-7.45 (m, 2H), 7.18-6.97 (m, 3H), 6.96-6.80 (m, 2H), 6.38 (dd, J=26.3, 2.3 Hz, 1H), 4.49 (d, J=19.6 Hz, 2H), 4.38-4.21 (m, 3H), 4.13 (s, 1H), 3.84 (t, J=5.8 Hz, 1H), 3.65-3.55 (m, 5H), 3.06-3.00 (m, 2H), 2.84 (dt, J=26.4, 5.9 Hz, 2H), 2.45-2.41 (m, 2H), 2.30 (q, J=7.5 Hz, 2H), 2.07-2.00 (m, 2H), 1.47-1.22 (m, 4H), 1.08 (d, J=9.9 Hz, 8H), 0.58 (t, J=7.5 Hz, 3H). MS (ESI) m/z 951.4 [M+H]⁺.

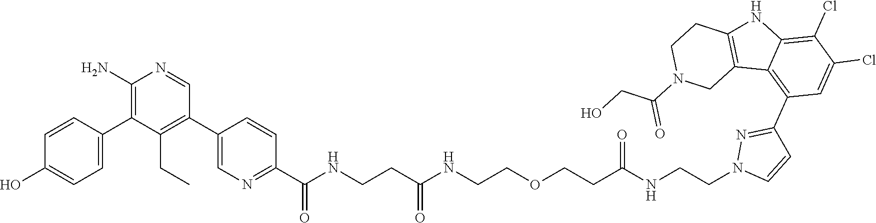 [SMILES: CCc1c(-c2ccc(C(=O)NCCC(=O)NCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 134: 6′-amino-N-(3-((2-(3-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-3-oxopropoxy)ethyl)amino)-3-oxopropyl)-4′-ethyl-5′-(4-hydroxyphenyl)-[3,3′-bipyridine]-6-carboxamide (KL186-152)

Example 134 was synthesized following similar procedure for preparing example 126 from intermediate 7 and linker 52. Light yellow solid, 5.1 mg, 56% yield. ¹H NMR (400 MHz, Methanol-d₄) δ 8.48 (dd, J=5.6, 2.2 Hz, 1H), 8.04 (d, J=8.1 Hz, 1H), 7.83 (dt, J=8.1, 2.5 Hz, 1H), 7.72-7.47 (m, 2H), 7.17-6.99 (m, 3H), 6.99-6.86 (m, 2H), 6.37 (dd, J=30.7, 2.3 Hz, 1H), 4.47 (d, J=9.3 Hz, 2H), 4.36-4.19 (m, 3H), 4.12 (s, 1H), 3.94-3.79 (m, 1H), 3.69-3.44 (m, 7H), 3.34 (q, J=4.9 Hz, 2H), 3.19 (s, 2H), 2.83 (dt, J=27.4, 5.9 Hz, 2H), 2.43 (t, J=6.5 Hz, 2H), 2.35-2.15 (m, 4H), 0.56 (t, J=7.5 Hz, 3H). MS (ESI) m/z 911.3 [M+H]⁺.

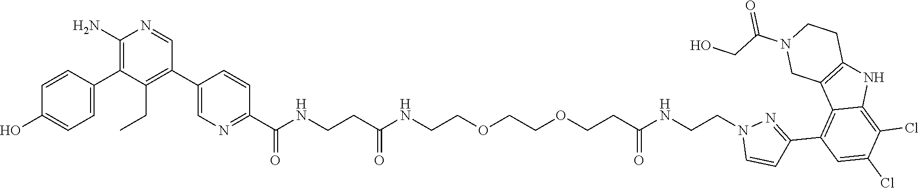 [SMILES: CCc1c(-c2ccc(C(=O)NCCC(=O)NCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 135: 6′-amino-N-(1-(3-(6,7-dichloro-2-(2-hydroxyacetyl)-2,3,4,5-tetrahydro-1H-pyrido[4,3-b]indol-9-yl)-1H-pyrazol-1-yl)-4,14-dioxo-7,10-dioxa-3,13-diazahexadecan-16-yl)-4′-ethyl-5′-(4-hydroxyphenyl)-[3,3′-bipyridine]-6-carboxamide (KL186-153)

Example 135 was synthesized following similar procedure for preparing example 126 from intermediate 7 and linker 53. Light yellow solid, 5.4 mg, 57% yield. ¹H NMR (400 MHz, Methanol-d₄) δ 8.49 (d, J=2.3 Hz, 1H), 8.05 (d, J=8.0 Hz, 1H), 7.85 (td, J=8.0, 7.5, 2.2 Hz, 1H), 7.73-7.49 (m, 2H), 7.15-7.00 (m, 3H), 6.94-6.83 (m, 2H), 6.37 (dd, J=30.5, 2.3 Hz, 1H), 4.48 (d, J=10.5 Hz, 2H), 4.25 (d, J=6.7 Hz, 3H), 4.13 (s, 1H), 3.92-3.77 (m, 1H), 3.69-3.51 (m, 7H), 3.38 (dd, J=14.3, 5.1 Hz, 6H), 3.24 (d, J=5.3 Hz, 2H), 2.84 (dt, J=26.6, 5.8 Hz, 2H), 2.43 (t, J=6.6 Hz, 2H), 2.38-2.18 (m, 4H), 0.57 (t, J=7.5 Hz, 3H). MS (ESI) m/z 954.3 [M+H]⁺.

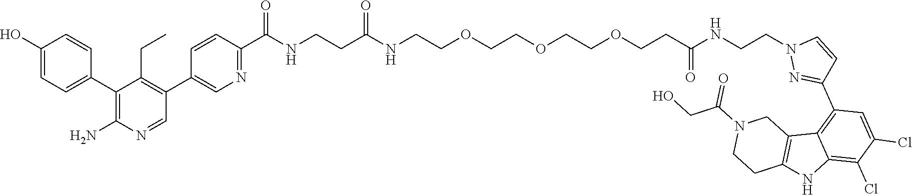 [SMILES: CCc1c(-c2ccc(C(=O)NCCC(=O)NCCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 136: 6′-amino-N-(1-(3-(6,7-dichloro-2-(2-hydroxyacetyl)-2,3,4,5-tetrahydro-1H-pyrido[4,3-b]indol-9-yl)-1H-pyrazol-1-yl)-4,17-dioxo-7,10,13-trioxa-3,16-diazanonadecan-19-yl)-4′-ethyl-5′-(4-hydroxyphenyl)-[3,3′-bipyridine]-6-carboxamide (KL186-154)

Example 136 was synthesized following similar procedure for preparing example 126 from intermediate 7 and linker 54. Light yellow solid, 6.0 mg, 60% yield. ¹H NMR (400 MHz, Methanol-d₄) δ 8.51 (d, J=2.5 Hz, 1H), 8.06 (d, J=8.0 Hz, 1H), 7.86 (dt, J=8.0, 2.2 Hz, 1H), 7.72-7.54 (m, 2H), 7.16-6.98 (m, 3H), 6.96-6.86 (m, 2H), 6.38 (dd, J=27.5, 2.3 Hz, 1H), 4.49 (d, J=15.3 Hz, 2H), 4.26 (dd, J=10.9, 4.9 Hz, 3H), 4.13 (s, 1H), 3.85 (q, J=7.6, 5.8 Hz, 1H), 3.65-3.54 (m, 7H), 3.44-3.35 (m, 9H), 3.24 (d, J=5.4 Hz, 3H), 2.84 (dt, J=26.0, 5.8 Hz, 2H), 2.43 (t, J=6.5 Hz, 2H), 2.37-2.19 (m, 4H), 0.57 (t, J=7.5 Hz, 3H). MS (ESI) m/z 999.4 [M+H]⁺.

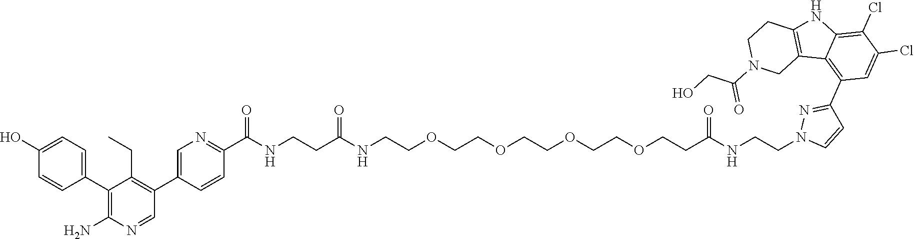 [SMILES: CCc1c(-c2ccc(C(=O)NCCC(=O)NCCOCCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 137: 6′-amino-N-(1-(3-(6,7-dichloro-2-(2-hydroxyacetyl)-2,3,4,5-tetrahydro-1H-pyrido[4,3-b]indol-9-yl)-1H-pyrazol-1-yl)-4,20-dioxo-7,10,13,16-tetraoxa-3,19-diazadocosan-22-yl)-4′-ethyl-5′-(4-hydroxyphenyl)-[3,3′-bipyridine]-6-carboxamide (KL186-155)

Example 137 was synthesized following similar procedure for preparing example 126 from intermediate 7 and linker 55. Light yellow solid, 5.3 mg, 51% yield. ¹H NMR (400 MHz, Methanol-d₄) δ 8.52 (d, J=2.3 Hz, 1H), 8.07 (d, J=8.0 Hz, 1H), 7.87 (dt, J=8.0, 2.2 Hz, 1H), 7.75-7.49 (m, 2H), 7.20-7.00 (m, 3H), 6.95-6.83 (m, 2H), 6.39 (dd, J=26.8, 2.4 Hz, 1H), 4.49 (d, J=15.4 Hz, 2H), 4.34-4.20 (m, 3H), 4.13 (s, 1H), 3.85 (t, J=5.8 Hz, 1H), 3.71-3.50 (m, 7H), 3.49-3.35 (m, 13H), 3.25 (d, J=5.3 Hz, 3H), 2.84 (dt, J=25.0, 5.8 Hz, 2H), 2.44 (t, J=6.5 Hz, 2H), 2.38-2.23 (m, 4H), 0.58 (t, J=7.4 Hz, 3H). MS (ESI) m/z 1043.4 [M+H]⁻.

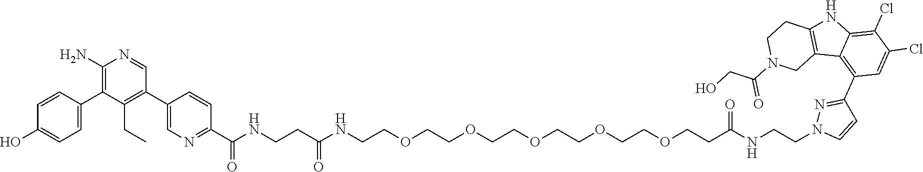 [SMILES: CCc1c(-c2ccc(C(=O)NCCC(=O)NCCOCCOCCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 138: 6′-amino-N-(1-(3-(6,7-dichloro-2-(2-hydroxyacetyl)-2,3,4,5-tetrahydro-1H-pyrido[4,3-b]indol-9-yl)-1H-pyrazol-1-yl)-4,23-dioxo-7,10,13,16,19-pentaoxa-3,22-diazapentacosan-25-yl)-4′-ethyl-5′-(4-hydroxyphenyl)-[3,3′-bipyridine]-6-carboxamide (KL186-156)

Example 138 was synthesized following similar procedure for preparing example 126 from intermediate 7 and linker 56. Light yellow solid, 5.9 mg, 54% yield. ¹H NMR (400 MHz, Methanol-d₄) δ 8.52 (d, J=2.1 Hz, 1H), 8.07 (d, J=8.0 Hz, 1H), 7.88 (dt, J=8.1, 2.3 Hz, 1H), 7.74-7.45 (m, 2H), 7.24-6.99 (m, 3H), 6.97-6.83 (m, 2H), 6.39 (dd, J=26.3, 2.3 Hz, 1H), 4.49 (d, J=14.9 Hz, 2H), 4.35-4.21 (m, 3H), 4.13 (s, 1H), 3.85 (t, J=5.8 Hz, 1H), 3.69-3.51 (m, 7H), 3.45-3.37 (m, 17H), 3.24 (t, J=5.4 Hz, 3H), 2.84 (dt, J=24.8, 5.9 Hz, 2H), 2.45 (t, J=6.5 Hz, 2H), 2.40-2.20 (m, 4H), 0.58 (t, J=7.5 Hz, 3H). MS (ESI) m/z 1087.4 [M+H]⁺.

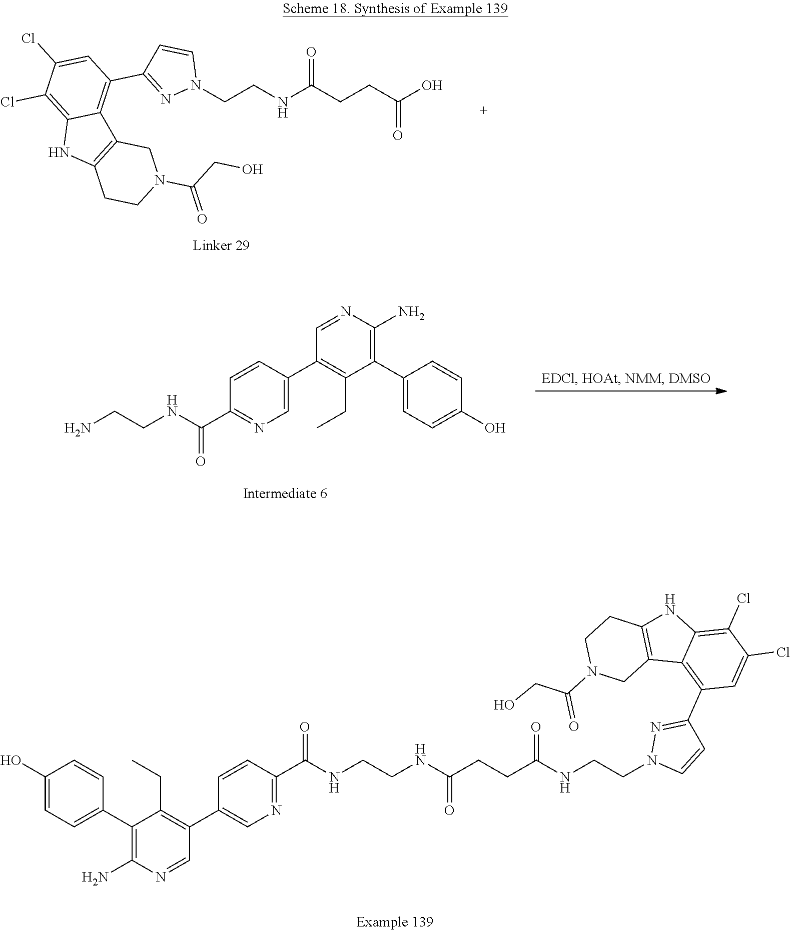 [SMILES: O=C(O)CCC(=O)NCCn1ccc(-c2cc(Cl)c(Cl)c3[nH]c4c(c23)CN(C(=O)CO)CC4)n1 ; CCc1c(-c2ccc(C(=O)NCCN)nc2)cnc(N)c1-c1ccc(O)cc1 ; CCc1c(-c2ccc(C(=O)NCCNC(=O)CCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 139: N1-(2-(6′-amino-4′-ethyl-5′-(4-hydroxyphenyl)-[3,3′-bipyridine]-6-carboxamido)ethyl)-N4-(2-(3-(6,7-dichloro-2-(2-hydroxyacetyl)-2,3,4,5-tetrahydro-1H-pyrido[4,3-b]indol-9-yl)-1H-pyrazol-1-yl)ethyl) succinamide (KL186-158)

To a solution of intermediate 6 (4 mg, 0.01 mmol) in DMSO (1 mL) were added linker 29 (5.1 mg, 0.01 mmol, 1.0 equiv), EDCI (1-ethyl-3-(3-dimethylaminopropyl)carbodiimide) (3.0 mg, 0.015 mmol, 1.5 equiv), HOAt (1-hydroxy-7-azabenzo-triazole) (2.0 mg, 0.015 mmol, 1.5 equiv), and NMM (N-Methylmorpholine) (3.0 mg, 0.03 mmol, 3.0 equiv). After being stirred overnight at room temperature, the resulting mixture was purified by preparative HPLC (5%-70% acetonitrile/0.1% TFA in H₂O) to afford titled compound as light yellow solid (5.1 mg, 58%). ¹H NMR (400 MHz, Methanol-d₄) δ 8.51 (s, 1H), 8.04 (d, J=7.8 Hz, 1H), 7.83 (dd, J=19.4, 8.0 Hz, 1H), 7.71-7.51 (m, 2H), 7.14-7.02 (m, 3H), 6.95-6.83 (m, 2H), 6.37 (dd, J=32.5, 2.2 Hz, 1H), 4.46 (d, J=4.1 Hz, 2H), 4.24 (dd, J=11.7, 5.8 Hz, 3H), 4.12 (s, 1H), 3.83 (d, J=5.8 Hz, 1H), 3.62 (t, J=5.7 Hz, 1H), 3.55 (d, J=5.5 Hz, 2H), 3.45 (d, J=6.4 Hz, 2H), 3.31 (d, J=5.5 Hz, 2H), 2.83 (dt, J=27.7, 5.9 Hz, 2H), 2.36 (d, J=4.1 Hz, 4H), 2.30-2.25 (m, 2H), 0.65-0.45 (m, 3H). MS (ESI) m/z 867.3 [M+H]⁺.

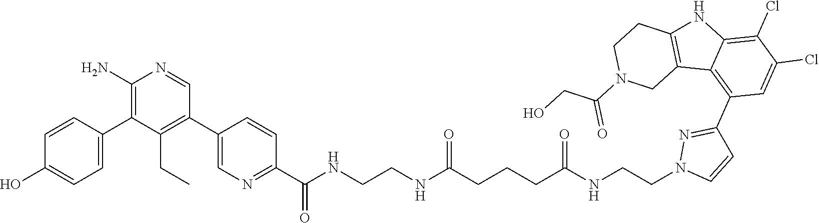 [SMILES: CCc1c(-c2ccc(C(=O)NCCNC(=O)CCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 114: N¹-(2-(6′-amino-4′-ethyl-5′-(4-hydroxyphenyl)-[3,3′-bipyridine]-6-carboxamido)ethyl)-N⁵-(2-(3-(6,7-dichloro-2-(2-hydroxyacetyl)-2,3,4,5-tetrahydro-1H-pyrido[4,3-b]indol-9-yl)-1H-pyrazol-1-yl)ethyl) glutaramide (KL186-159)

Example 140 was synthesized following similar procedure for preparing example 139 from intermediate 6 and linker 30. Light yellow solid, 4.9 mg, 52% yield. ¹H NMR (400 MHz, Methanol-d₄) δ 8.51 (dd, J=9.8, 2.2 Hz, 1H), 8.03 (d, J=8.0 Hz, 1H), 7.80 (dd, J=8.0, 2.2 Hz, 1H), 7.71-7.49 (m, 2H), 7.19-6.99 (m, 3H), 6.99-6.81 (m, 2H), 6.38 (dd, J=33.2, 2.3 Hz, 1H), 4.46 (d, J=10.4 Hz, 2H), 4.26 (q, J=4.0, 2.6 Hz, 3H), 4.11 (s, 1H), 3.84 (t, J=5.9 Hz, 1H), 3.64-3.56 (m, 3H), 3.44 (t, J=5.7 Hz, 2H), 3.30 (t, J=5.7 Hz, 2H), 2.83 (dt, J=25.6, 5.9 Hz, 2H), 2.27 (q, J=7.5 Hz, 2H), 2.09-2.03 (m, 4H), 1.77-1.70 (m, 2H), 0.55 (t, J=7.5 Hz, 3H). MS (ESI) m/z 881.3 [M+H]⁻.

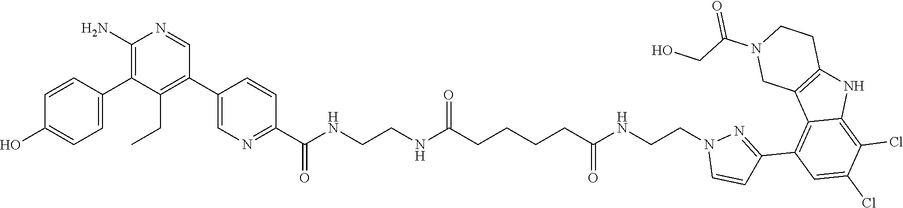 [SMILES: CCc1c(-c2ccc(C(=O)NCCNC(=O)CCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 141: N¹-(2-(6′-amino-4′-ethyl-5′-(4-hydroxyphenyl)-[3,3′-bipyridine]-6-carboxamido)ethyl)-N⁶-(2-(3-(6,7-dichloro-2-(2-hydroxyacetyl)-2,3,4,5-tetrahydro-1H-pyrido[4,3-b]indol-9-yl)-1H-pyrazol-1-yl)ethyl) adipamide (KL186-160)

Example 141 was synthesized following similar procedure for preparing example 139 from intermediate 6 and linker 31. Light yellow solid, 5.1 mg, 57% yield. ¹H NMR (400 MHz, Methanol-d₄) δ 8.53 (d, J=2.5 Hz, 1H), 8.05 (dd, J=8.1, 4.8 Hz, 1H), 7.84 (td, J=8.5, 2.2 Hz, 1H), 7.74-7.43 (m, 2H), 7.18-6.98 (m, 3H), 6.95-6.82 (m, 2H), 6.38 (dd, J=28.7, 2.3 Hz, 1H), 4.47 (d, J=11.2 Hz, 2H), 4.27 (d, J=7.3 Hz, 3H), 4.12 (s, 1H), 3.94-3.74 (m, 1H), 3.61 (dt, J=23.0, 5.8 Hz, 3H), 3.50-3.39 (m, 2H), 3.31 (q, J=6.1 Hz, 2H), 2.84 (dt, J=26.8, 5.8 Hz, 2H), 2.29 (q, J=7.5 Hz, 2H), 2.11-1.96 (m, 4H), 1.47-1.43 (m, 4H), 0.57 (t, J=7.5 Hz, 3H). MS (ESI) m/z 895.3 [M+H]⁺.

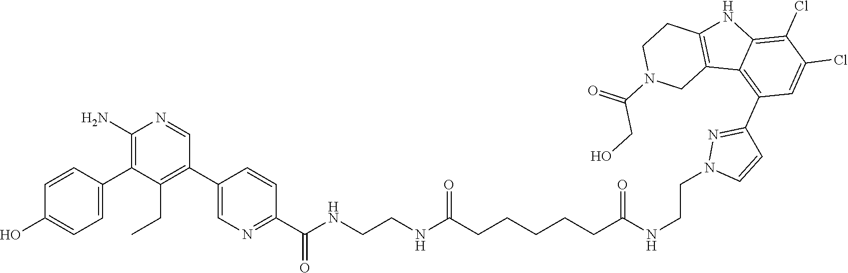 [SMILES: CCc1c(-c2ccc(C(=O)NCCNC(=O)CCCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 142: N¹-(2-(6′-amino-4′-ethyl-5′-(4-hydroxyphenyl)-[3,3′-bipyridine]-6-carboxamido)ethyl)-N⁷-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)heptanediamide (KL186-161)

Example 142 was synthesized following similar procedure for preparing example 139 from intermediate 6 and linker 32. Light yellow solid, 4.5 mg, 49% yield. ¹H NMR (400 MHz, Methanol-d₄) δ 8.53 (dd, J=5.7, 2.2 Hz, 1H), 8.06 (dd, J=8.1, 3.4 Hz, 1H), 7.85 (ddd, J=10.3, 8.0, 2.2 Hz, 1H), 7.67 (d, J=7.5 Hz, 1H), 7.56 (dd, J=23.0, 2.3 Hz, 1H), 7.19-7.02 (m, 3H), 6.93-6.82 (m, 2H), 6.38 (dd, J=27.8, 2.3 Hz, 1H), 4.47 (d, J=12.6 Hz, 2H), 4.26 (d, J=5.0 Hz, 3H), 4.14 (s, 1H), 3.85 (t, J=5.8 Hz, 1H), 3.70-3.51 (m, 3H), 3.51-3.40 (m, 2H), 3.36-3.32 (m, 2H), 2.84 (dt, J=25.1, 5.7 Hz, 2H), 2.32-2.26 (m, 2H), 2.10-1.95 (m, 4H), 1.46-1.36 (m, 4H), 1.18-1.10 (m, 2H), 0.60-0.55 (m, 3H). MS (ESI) m/z 909.3 [M+H]⁺.

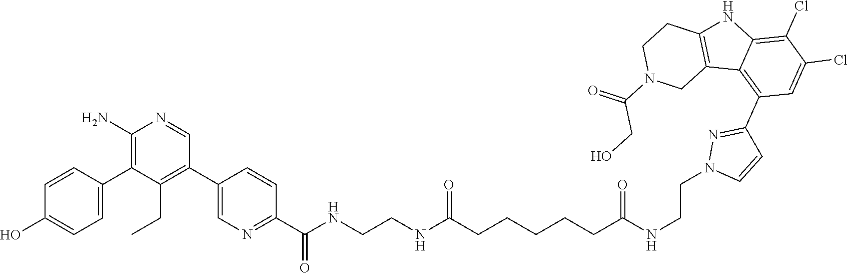 [SMILES: CCc1c(-c2ccc(C(=O)NCCNC(=O)CCCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 143: N¹-(2-(6′-amino-4′-ethyl-5′-(4-hydroxyphenyl)-[3,3′-bipyridine]-6-carboxamido)ethyl)-N⁸-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)octanediamide (KL186-162)

Example 143 was synthesized following similar procedure for preparing example 1139 from intermediate 6 and linker 33. Light yellow solid, 5.2 mg, 56% yield. ¹H NMR (400 MHz, Methanol-d₄) δ 8.54 (d, J=2.2 Hz, 1H), 8.06 (d, J=8.0 Hz, 1H), 7.87 (dd, J=8.1, 2.1 Hz, 1H), 7.67 (s, 1H), 7.55 (dd, J=24.2, 2.3 Hz, 1H), 7.19-6.99 (m, 3H), 6.97-6.83 (m, 2H), 6.39 (dd, J=27.4, 2.3 Hz, 1H), 4.48 (d, J=14.1 Hz, 2H), 4.33-4.19 (m, 3H), 4.13 (s, 1H), 3.85 (t, J=5.9 Hz, 1H), 3.64-3.55 (m, 3H), 3.47-3.43 (m, 2H), 3.41-3.30 (m, 2H), 2.84 (dt, J=24.9, 5.8 Hz, 2H), 2.30 (q, J=7.5 Hz, 2H), 2.06-2.01 (m, 4H), 1.41 (dd, J=13.8, 7.1 Hz, 4H), 1.22-0.97 (m, 4H), 0.58 (t, J=7.5 Hz, 3H). MS (ESI) m/z 923.4 [M+H]⁺.

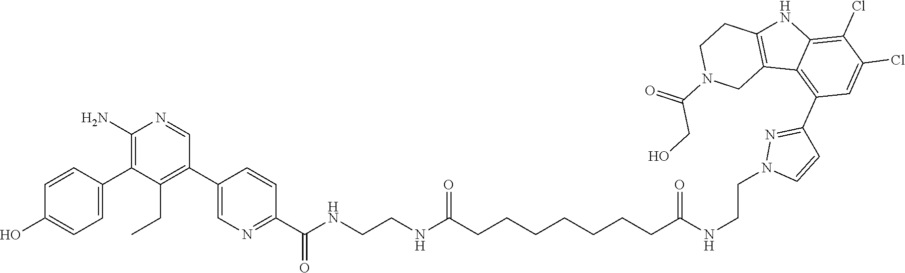 [SMILES: CCc1c(-c2ccc(C(=O)NCCNC(=O)CCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 144: N¹-(2-(6′-amino-4′-ethyl-5′-(4-hydroxyphenyl)-[3,3′-bipyridine]-6-carboxamido)ethyl)-N⁹-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)nonanediamide (KL186-163)

Example 144 was synthesized following similar procedure for preparing example 139 from intermediate 6 and linker 34. Light yellow solid, 6.0 mg, 64% yield. ¹H NMR (400 MHz, Methanol-d₄) δ 8.54 (d, J=2.1 Hz, 1H), 8.07 (dd, J=8.1, 2.1 Hz, 1H), 7.88 (td, J=8.1, 7.4, 2.2 Hz, 1H), 7.67 (d, J=1.4 Hz, 1H), 7.55 (dd, J=23.5, 2.3 Hz, 1H), 7.20-6.97 (m, 3H), 6.96-6.80 (m, 2H), 6.38 (dd, J=27.3, 2.3 Hz, 1H), 4.48 (d, J=14.7 Hz, 2H), 4.27 (dd, J=11.9, 6.0 Hz, 3H), 4.14 (s, 1H), 3.85 (t, J=5.9 Hz, 1H), 3.65-3.55 (m, 3H), 3.47-3.44 (m, 2H), 3.34 (q, J=5.5 Hz, 2H), 2.84 (dt, J=25.0, 5.9 Hz, 2H), 2.31 (q, J=7.5 Hz, 2H), 2.06-2.00 (m, 4H), 1.44-1.37 (m, 4H), 1.09 (d, J=4.9 Hz, 6H), 0.58 (t, J=7.5 Hz, 3H). MS (ESI) m/z 937.4 [M+H]⁺.

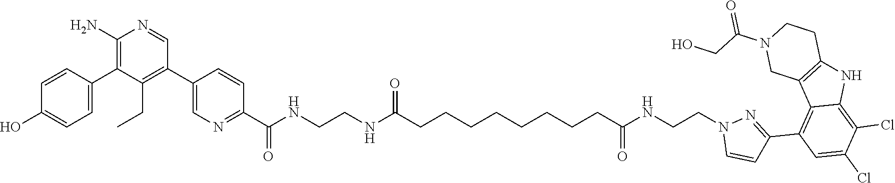 [SMILES: CCc1c(-c2ccc(C(=O)NCCNC(=O)CCCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 145: N¹-(2-(6′-amino-4′-ethyl-5′-(4-hydroxyphenyl)-[3,3′-bipyridine]-6-carboxamido)ethyl)-N¹⁰-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)decanediamide (KL186-164)

Example 145 was synthesized following similar procedure for preparing example 139 from intermediate 6 and linker 35. Light yellow solid, 5.2 mg, 55% yield. ¹H NMR (400 MHz, Methanol-d₄) δ 8.54 (d, J=2.2 Hz, 1H), 8.07 (d, J=8.0 Hz, 1H), 7.88 (ddd, J=8.0, 4.1, 2.1 Hz, 1H), 7.65 (s, 1H), 7.55 (dd, J=23.4, 2.3 Hz, 1H), 7.17-6.94 (m, 3H), 6.95-6.80 (m, 2H), 6.38 (dd, J=26.8, 2.3 Hz, 1H), 4.49 (d, J=17.3 Hz, 2H), 4.36-4.22 (m, 3H), 4.14 (s, 1H), 3.85 (t, J=5.8 Hz, 1H), 3.65-3.55 (m, 3H), 3.47-3.44 (m, 2H), 3.40-3.29 (m, 2H), 2.84 (dt, J=25.0, 5.8 Hz, 2H), 2.30 (q, J=7.5 Hz, 2H), 2.08-2.00 (m, 4H), 1.44-1.36 (m, 4H), 1.08 (t, J=10.9 Hz, 8H), 0.58 (t, J=7.5 Hz, 3H). MS (ESI) m/z 951.4 [M+H]⁺.

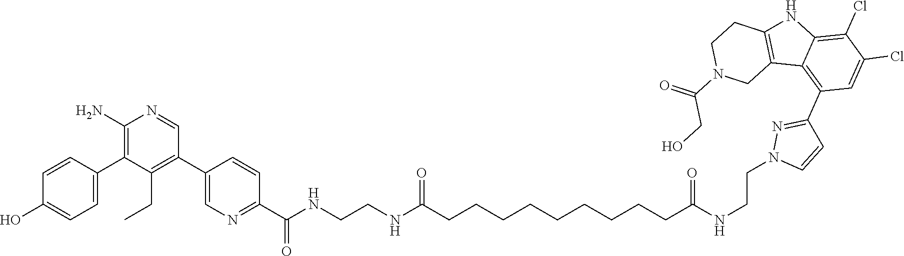 [SMILES: CCc1c(-c2ccc(C(=O)NCCNC(=O)CCCCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 146: N¹-(2-(6′-amino-4′-ethyl-5′-(4-hydroxyphenyl)-[3,3′-bipyridine]-6-carboxamido)ethyl)-N¹¹-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)undecanediamide (KL186-165)

Example 146 was synthesized following similar procedure for preparing example 139 from intermediate 6 and linker 28. Light yellow solid, 4.8 mg, 50% yield. ¹H NMR (400 MHz, Methanol-d₄) δ 8.54 (d, J=2.2 Hz, 1H), 8.08 (d, J=8.0 Hz, 1H), 7.95-7.81 (m, 1H), 7.66 (s, 1H), 7.55 (dd, J=23.8, 2.3 Hz, 1H), 7.13-7.02 (m, 3H), 6.95-6.87 (m, 2H), 6.38 (dd, J=26.8, 2.3 Hz, 1H), 4.49 (d, J=18.9 Hz, 2H), 4.31-4.22 (m, 3H), 4.13 (s, 1H), 3.84 (t, J=5.8 Hz, 1H), 3.65-3.55 (m, 3H), 3.46 (t, J=5.8 Hz, 2H), 3.41-3.32 (m, 2H), 2.84 (dt, J=25.4, 5.9 Hz, 2H), 2.31 (q, J=7.4 Hz, 2H), 2.08-2.00 (m, 4H), 1.40 (q, J=7.8, 7.3 Hz, 4H), 1.14-1.01 (m, 10H), 0.59 (t, J=7.5 Hz, 3H). MS (ESI) m/z 965.4 [M+H]⁺.

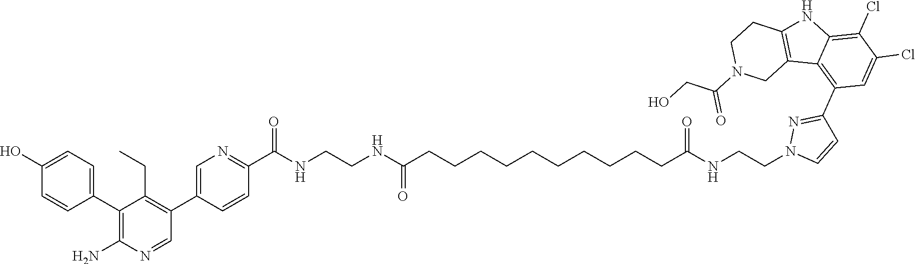 [SMILES: CCc1c(-c2ccc(C(=O)NCCNC(=O)CCCCCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 147: N¹-(2-(6′-amino-4′-ethyl-5′-(4-hydroxyphenyl)-[3,3′-bipyridine]-6-carboxamido)ethyl)-N¹²-(2-(3-(6,7-dichloro-2-(2-hydroxyacetyl)-2,3,4,5-tetrahydro-1H-pyrido[4,3-b]indol-9-yl)-1H-pyrazol-1-yl)ethyl) dodecanediamide (KL186-166)

Example 147 was synthesized following similar procedure for preparing example 139 from intermediate 6 and linker 36. Light yellow solid, 5.8 mg, 59% yield. ¹H NMR (400 MHz, Methanol-d₄) δ 8.54 (d, J=2.2 Hz, 1H), 8.08 (d, J=8.0 Hz, 1H), 7.89 (ddd, J=7.9, 4.9, 2.1 Hz, 1H), 7.66 (s, 1H), 7.55 (dd, J=24.0, 2.3 Hz, 1H), 7.16-7.01 (m, 3H), 6.98-6.83 (m, 2H), 6.38 (dd, J=27.0, 2.3 Hz, 1H), 4.49 (d, J=20.5 Hz, 2H), 4.33-4.20 (m, 3H), 4.13 (s, 1H), 3.93-3.79 (m, 1H), 3.65-3.54 (m, 3H), 3.53-3.42 (m, 2H), 3.35 (t, J=5.9 Hz, 2H), 2.84 (dt, J=25.2, 5.8 Hz, 2H), 2.31 (q, J=7.4 Hz, 2H), 2.09-1.99 (m, 4H), 1.58-1.33 (m, 4H), 1.17-1.00 (m, 12H), 0.59 (t, J=7.4 Hz, 3H). MS (ESI) m/z 979.4 [M+H]⁺.

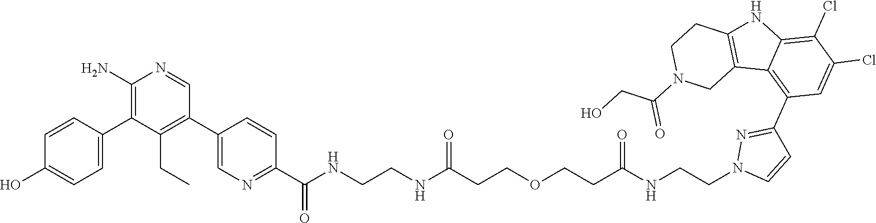 [SMILES: CCc1c(-c2ccc(C(=O)NCCNC(=O)CCOCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 148: 6′-amino-N-(2-(3-(3-((2-(3-(6,7-dichloro-2-(2-hydroxyacetyl)-2,3,4,5-tetrahydro-1H-pyrido[4,3-b]indol-9-yl)-1H-pyrazol-1-yl)ethyl)amino)-3-oxopropoxy)propanamido)ethyl)-4′-ethyl-5′-(4-hydroxyphenyl)-[3,3′-bipyridine]-6-carboxamide (KL186-167)

Example 148 was synthesized following similar procedure for preparing example 139 from intermediate 6 and linker 37. Light yellow solid, 4.8 mg, 54% yield. ¹H NMR (400 MHz, Methanol-d₄) δ 8.49 (t, J=2.7 Hz, 1H), 8.04 (d, J=8.0 Hz, 1H), 7.82 (dd, J=8.1, 2.1 Hz, 1H), 7.66 (d, J=3.8 Hz, 1H), 7.57 (dd, J=24.2, 2.3 Hz, 1H), 7.16-7.01 (m, 3H), 6.97-6.78 (m, 2H), 6.37 (dd, J=27.9, 2.3 Hz, 1H), 4.47 (d, J=12.1 Hz, 2H), 4.34-4.22 (m, 3H), 4.13 (s, 1H), 3.83 (t, J=5.9 Hz, 1H), 3.63-3.57 (m, 3H), 3.55-3.49 (m, 4H), 3.44 (t, J=5.9 Hz, 2H), 3.33 (dd, J=6.7, 4.8 Hz, 2H), 2.83 (dt, J=26.2, 5.9 Hz, 2H), 2.36-2.21 (m, 6H), 0.55 (t, J=7.5 Hz, 3H). MS (ESI) m/z 911.3 [M+H]⁺.

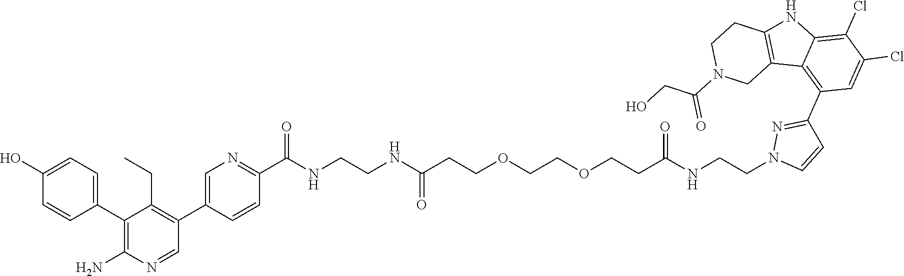 [SMILES: CCc1c(-c2ccc(C(=O)NCCNC(=O)CCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 149: 6′-amino-N-(16-(3-(6,7-dichloro-2-(2-hydroxyacetyl)-2,3,4,5-tetrahydro-1H-pyrido[4,3-b]indol-9-yl)-1H-pyrazol-1-yl)-4,13-dioxo-7,10-dioxa-3,14-diazahexadecyl)-4′-ethyl-5′-(4-hydroxyphenyl)-[3,3′-bipyridine]-6-carboxamide (KL186-168)

Example 149 was synthesized following similar procedure for preparing example 139 from intermediate 6 and linker 38. Light yellow solid, 5.5 mg, 58% yield. ¹H NMR (400 MHz, Methanol-d₄) δ 8.52 (d, J=2.2 Hz, 1H), 8.06 (d, J=8.0 Hz, 1H), 7.85 (dt, J=8.0, 2.1 Hz, 1H), 7.72-7.52 (m, 2H), 7.21-7.02 (m, 3H), 6.97-6.81 (m, 2H), 6.38 (dd, J=29.5, 2.3 Hz, 1H), 4.49 (d, J=11.1 Hz, 2H), 4.26 (d, J=7.1 Hz, 3H), 4.14 (s, 1H), 3.93-3.78 (m, 1H), 3.67-3.53 (m, 7H), 3.42 (q, J=6.6 Hz, 6H), 3.37-3.29 (m, 2H), 2.84 (dt, J=25.3, 5.9 Hz, 2H), 2.43-2.21 (m, 6H), 0.58 (t, J=7.5 Hz, 3H). MS (ESI) m/z 955.3 [M+H]⁺.

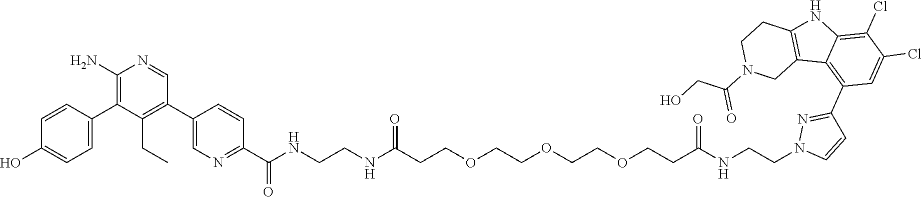 [SMILES: CCc1c(-c2ccc(C(=O)NCCNC(=O)CCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 150: 6′-amino-N-(19-(3-(6,7-dichloro-2-(2-hydroxyacetyl)-2,3,4,5-tetrahydro-1H-pyrido[4,3-b]indol-9-yl)-1H-pyrazol-1-yl)-4,16-dioxo-7,10,13-trioxa-3,17-diazanonadecyl)-4′-ethyl-5′-(4-hydroxyphenyl)-[3,3′-bipyridine]-6-carboxamide (KL186-169)

Example 150 was synthesized following similar procedure for preparing example 139 from intermediate 6 and linker 39. Light yellow solid, 4.7 mg, 47% yield. ¹H NMR (400 MHz, Methanol-d₄) δ 8.53 (d, J=2.2 Hz, 1H), 8.06 (d, J=8.0 Hz, 1H), 7.86 (dt, J=8.3, 2.6 Hz, 1H), 7.74-7.49 (m, 2H), 7.19-6.99 (m, 3H), 6.95-6.82 (m, 2H), 6.38 (dd, J=27.6, 2.3 Hz, 1H), 4.49 (d, J=14.9 Hz, 2H), 4.35-4.20 (m, 3H), 4.13 (s, 1H), 3.84 (t, J=5.8 Hz, 1H), 3.69-3.53 (m, 7H), 3.49-3.36 (m, 10H), 3.37-3.28 (m, 2H), 2.84 (dt, J=24.8, 5.8 Hz, 2H), 2.34-2.26 (m, 6H), 0.57 (t, J=7.5 Hz, 3H). MS (ESI) m/z 999.4 [M+H]⁺.

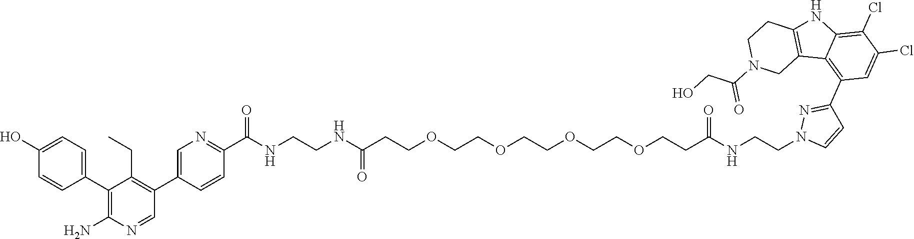 [SMILES: CCc1c(-c2ccc(C(=O)NCCNC(=O)CCOCCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 151: N¹-(2-(6′-amino-4′-ethyl-5′-(4-hydroxyphenyl)-[3,3′-bipyridine]-6-carboxamido)ethyl)-N¹⁶-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-4,7,10,13-tetraoxahexadecanediamide (KL186-170)

Example 151 was synthesized following similar procedure for preparing example 139 from intermediate 6 and linker 40. Light yellow solid, 5.8 mg, 56% yield. ¹H NMR (400 MHz, Methanol-d₄) δ 8.54 (d, J=2.2 Hz, 1H), 8.07 (d, J=8.0 Hz, 1H), 7.88 (dt, J=8.0, 2.3 Hz, 1H), 7.73-7.48 (m, 2H), 7.21-6.97 (m, 3H), 6.97-6.80 (m, 2H), 6.39 (dd, J=26.7, 2.3 Hz, 1H), 4.50 (d, J=14.3 Hz, 2H), 4.34-4.20 (m, 3H), 4.13 (s, 1H), 3.95-3.78 (m, 1H), 3.69-3.52 (m, 7H), 3.50-3.37 (m, 14H), 3.37-3.28 (m, 2H), 2.84 (dt, J=24.4, 5.8 Hz, 2H), 2.35-2.27 (m, 6H), 0.58 (t, J=7.4 Hz, 3H). MS (ESI) m/z 1043.5 [M+H]⁺.

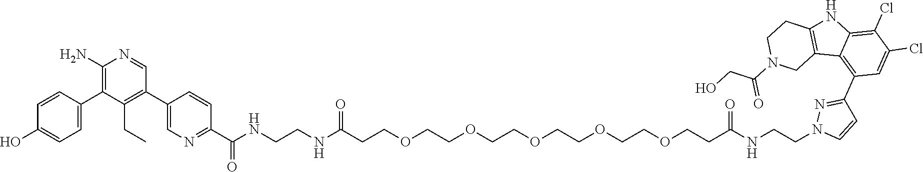 [SMILES: CCc1c(-c2ccc(C(=O)NCCNC(=O)CCOCCOCCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1]

### Example 152: N¹-(2-(6′-amino-4′-ethyl-5′-(4-hydroxyphenyl)-[3,3′-bipyridine]-6-carboxamido)ethyl)-N¹⁹-(2-(3-(6,7-dichloro-2-(2-hydroxyacetyl)-2,3,4,5-tetrahydro-1H-pyrido[4,3-b]indol-9-yl)-1H-pyrazol-1-yl)ethyl)-4,7,10,13,16-pentaoxanonadecanediamide (KL186-171)

Example 152 was synthesized following similar procedure for preparing example 139 from intermediate 6 and linker 41. Light yellow solid, 5.1 mg, 47% yield. ¹H NMR (400 MHz, Methanol-d₄) δ 8.55 (d, J=2.2 Hz, 1H), 8.08 (d, J=8.0 Hz, 1H), 7.88 (dd, J=8.1, 2.1 Hz, 1H), 7.68 (s, 1H), 7.60 (dd, J=20.1, 2.3 Hz, 1H), 7.19-7.01 (m, 3H), 6.96-6.79 (m, 2H), 6.39 (dd, J=26.1, 2.3 Hz, 1H), 4.50 (d, J=14.2 Hz, 2H), 4.34-4.22 (m, 3H), 4.13 (s, 1H), 3.86 (q, J=6.1 Hz, 1H), 3.68-3.55 (m, 7H), 3.51-3.38 (m, 18H), 3.38-3.30 (m, 2H), 2.84 (dt, J=24.3, 5.8 Hz, 2H), 2.41-2.23 (m, 6H), 0.59 (t, J=7.4 Hz, 3H). MS (ESI) m/z 1089.5 [M+H]⁺.

### Procedure for the Synthesis of CFTR Based Bivalent Compounds

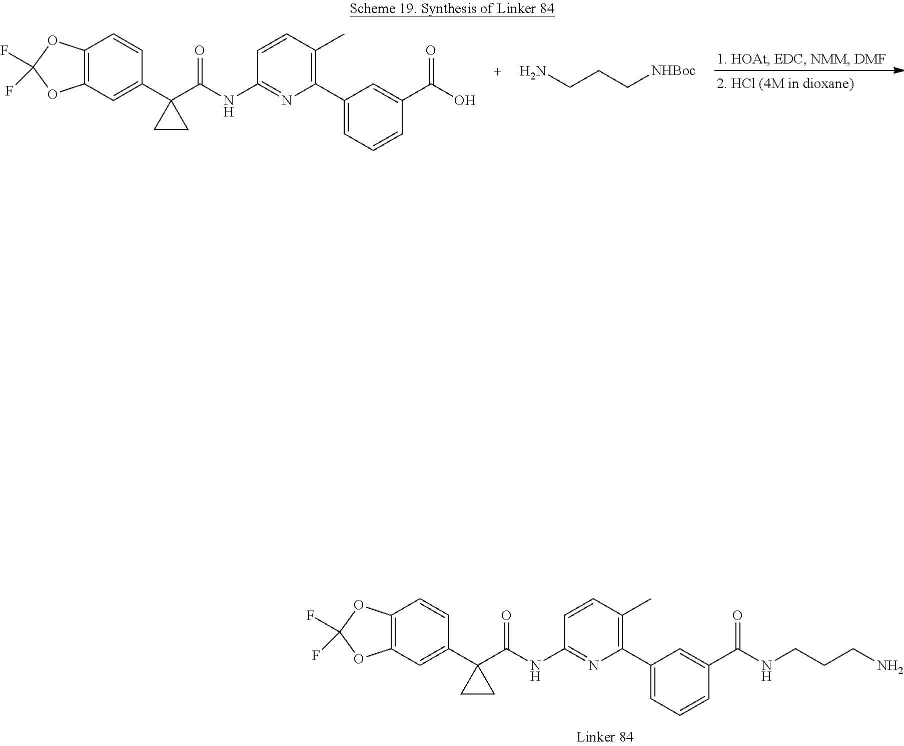

### Linker 84: N-(3-aminopropyl)-3-(6-(1-(2,2-difluorobenzo[d][1,3]dioxol-5-yl)cyclopropane-1-carboxamido)-3-methylpyridin-2-yl)benzamide

3-(6-(1-(2,2-difluorobenzo[d][1,3]dioxol-5-yl)cyclopropane-1-carboxamido)-3-methylpyridin-2-yl)benzoic acid (100 mg, 0.22 mmol, 1.0 eq) (prepared following previous reported procedure) (Henning et al., 2022), tert-butyl (3-aminopropyl)carbamate (43 mg, 0.24 mmol, 1.1 eq), EDC·HCl (63 mg, 0.33 mmol, 1.5 eq), HOAt (45 mg, 0.33 mmol, 1.5 eq), NMM (67 mg, 0.66 mmol, 3.0 eq) were stirred in 1.5 mL DMF at room temperature for 2 h. Then the mixture was purified via RP-C18 column [MeOH/Water (0.1% TFA)]. Then the product was dissolved in 1 mL MeOH and 1 mL HCl (4M in dioxane), the mixture was stirred at room temperature for 2 h. Then removed all the volatiles, the product was obtained as a white solid (50 mg, 45% yield over two steps). ¹H NMR (400 MHz, CD₃OD) δ 8.12 (d, J=9.0 Hz, 1H), 8.08-8.00 (m, 3H), 7.75-7.66 (m, 2H), 7.41 (s, 1H), 7.35 (d, J=8.3 Hz, 1H), 7.25 (d, J=8.3 Hz, 1H), 3.53 (t, J=5.9 Hz, 2H), 3.02 (t, J=7.3 Hz, 2H), 2.35 (s, 3H), 1.99 (p, J=6.9 Hz, 2H), 1.75 (s, 2H), 1.35 (s, 2H). MS (ESI) [M+H]⁺=509.1.

Linkers 85-97 are synthesized as the same procedure for preparing linker 84.

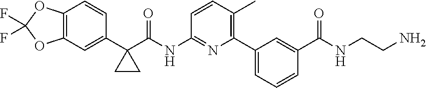 [SMILES: Cc1ccc(NC(=O)C2(c3ccc4c(c3)OC(F)(F)O4)CC2)nc1-c1cccc(C(=O)NCCN)c1]

### Linker 85: N-(2-aminoethyl)-3-(6-(1-(2,2-difluorobenzo[d][1,3]dioxol-5-yl)cyclopropane-1-carboxamido)-3-methylpyridin-2-yl)benzamide

White solid, 61% yield. ¹H NMR (400 MHz, CD₃OD) δ 8.13 (d, J=8.8 Hz, 1H), 8.07 (d, J=7.6 Hz, 1H), 8.02-7.98 (m, 2H), 7.70-7.63 (m, 2H), 7.37-7.30 (m, 2H), 7.23 (d, J=10.8 Hz, 1H), 3.80-3.77 (m, 2H), 3.31 (s, 2H), 2.36 (s, 3H), 1.75 (s, 2H), 1.31 (t, J=3.2 Hz, 2H). MS (TOF) [M+H]⁺=495.5.

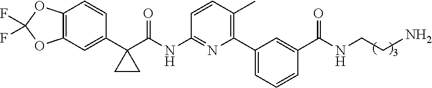

### Linker 86: N-(4-aminobutyl)-3-(6-(1-(2,2-difluorobenzo[d][1,3]dioxol-5-yl)cyclopropane-1-carboxamido)-3-methylpyridin-2-yl)benzamide

White solid, 60% yield. ¹H NMR (400 MHz, CD₃OD) δ 8.09 (d, J=8.4 Hz, 1H), 7.89 (d, J=5.4 Hz, 1H), 7.84 (s, 1H), 7.80 (d, J=8.6 Hz, 1H), 7.60-7.55 (m, 2H), 7.40 (s, 1H), 7.34 (d, J=8.3 Hz, 1H), 7.23 (d, J=8.3 Hz, 1H), 3.44 (s, 2H), 2.99 (s, 2H), 2.26 (s, 3H), 1.77-1.66 (m, 6H), 1.27 (s, 2H). MS (ESI) [M+H]⁺=523.5.

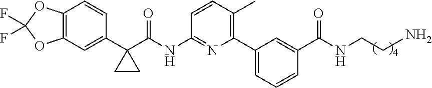

### Linker 87: N-(5-aminopentyl)-3-(6-(1-(2,2-difluorobenzo[d][1,3]dioxol-5-yl)cyclopropane-1-carboxamido)-3-methylpyridin-2-yl)benzamide

White solid, 65% yield. ¹H NMR (400 MHz, CD₃OD) δ 8.19-7.84 (m, 4H), 7.67-7.53 (m, 2H), 7.30 (s, 1H), 7.24 (d, J=7.9 Hz, 1H), 7.15 (d, J=8.9 Hz, 1H), 3.33 (t, J=7.8 Hz, 2H), 2.84 (t, J=7.6 Hz, 2H), 2.31-2.20 (m, 3H), 1.69-1.54 (m, 6H), 1.45-1.35 (m, 2H), 1.30-1.21 (m, 2H). MS (ESI) [M+H]⁺=537.2.

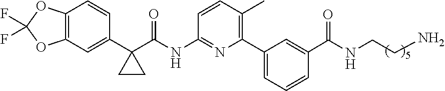

### Linker 88: N-(6-aminohexyl)-3-(6-(1-(2,2-difluorobenzo[d][1,3]dioxol-5-yl)cyclopropane-1-carboxamido)-3-methylpyridin-2-yl)benzamide

White solid, 62% yield. ¹H NMR (400 MHz, CD₃OD) § 8.39 (d, J=8.0 Hz, 1H), 8.16-8.06 (m, 3H), 7.80-7.70 (m, 2H), 7.35-7.30 (m, 2H), 7.20-7.18 (m, 1H), 3.38-3.34 (m, 2H), 2.91 (s, 2H), 2.38 (s, 3H), 1.75-1.63 (m, 6H), 1.42-1.34 (m, 8H). MS (ESI) [M+H]⁺=551.4.

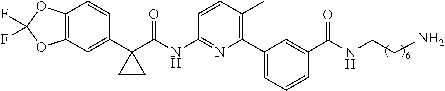

### Linker 89: N-(7-aminoheptyl)-3-(6-(1-(2,2-difluorobenzo[d][1,3]dioxol-5-yl)cyclopropane-1-carboxamido)-3-methylpyridin-2-yl)benzamide

White solid, 64% yield. ¹H NMR (400 MHz, CD₃OD) δ 8.38 (d, J=8.0 Hz, 1H), 8.12-8.04 (m, 3H), 7.81-7.70 (m, 2H), 7.35-7.30 (m, 2H), 7.19 (d, J=7.2 Hz, 1H), 3.37-3.34 (s, 2H), 2.89 (s, 2H), 2.38 (s, 3H), 1.74-1.61 (m, 6H), 1.38-1.34 (m, 8H). MS (TOF) [M+H]⁺=565.7.

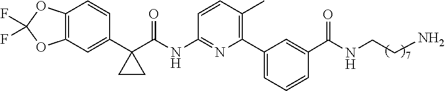

### Linker 90: N-(8-aminooctyl)-3-(6-(1-(2,2-difluorobenzo[d][1,3]dioxol-5-yl)cyclopropane-1-carboxamido)-3-methylpyridin-2-yl)benzamide

White solid, 60% yield. ¹H NMR (400 MHz, CD₃OD) δ 8.38 (d, J=8.0 Hz, 1H), 8.14-8.04 (m, 3H), 7.81-7.70 (m, 2H), 7.35-7.30 (m, 2H), 7.19-7.18 (m, 1H), 3.36-3.34 (m, 2H), 2.89 (t, J=7.6 Hz, 2H), 2.38 (s, 3H), 1.80-1.53 (m, 6H), 1.35 (s, 10H). MS (TOF) [M+H]⁺=579.6.

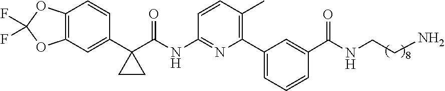

### Linker 91: N-(9-aminononyl)-3-(6-(1-(2,2-difluorobenzo[d][1,3]dioxol-5-yl)cyclopropane-1-carboxamido)-3-methylpyridin-2-yl)benzamide

White solid, 59% yield. ¹H NMR (400 MHz, CD₃OD) § 8.38 (d, J=8.0 Hz, 1H), 8.14-8.04 (m, 3H), 7.81-7.70 (m, 2H), 7.35-7.30 (m, 2H), 7.20-7.18 (m, 1H), 3.35-3.37 (m, 2H), 2.89 (t, J=8.0 Hz, 2H), 2.38 (s, 3H), 1.75 (s, 2H), 1.66-1.58 (m, 4H), 1.34-1.33 (m, 12H). MS (TOF) [M+H]⁺=593.5.

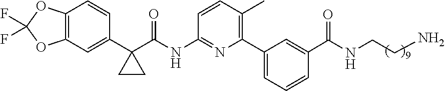

### Linker 92: N-(10-aminodecyl)-3-(6-(1-(2,2-difluorobenzo[d][1,3]dioxol-5-yl)cyclopropane-1-carboxamido)-3-methylpyridin-2-yl)benzamide

White solid, 64% yield. ¹H NMR (400 MHz, CD₃OD) δ 8.39 (s, 1H), 8.18-8.05 (m, 3H), 7.82-7.69 (m, 2H), 7.33-7.30 (m, 2H), 7.18 (s, 1H), 3.34-7.32 (m, 2H), 2.89 (s, 2H), 2.39 (s, 3H), 1.75-1.59 (m, 6H), 1.33-1.30 (m, 14H). MS (TOF) [M+H]⁺=607.5.

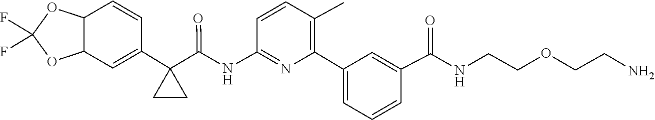 [SMILES: Cc1ccc(NC(=O)C2(C3=CC4OC(F)(F)OC4C=C3)CC2)nc1-c1cccc(C(=O)NCCOCCN)c1]

### Linker 93: N-(2-(2-aminoethoxy)ethyl)-3-(6-(1-(2,2-difluorobenzo[d][1,3]dioxol-5-yl)cyclopropane-1-carboxamido)-3-methylpyridin-2-yl)benzamide

White solid, 62% yield. 1H NMR (400 MHz, CD₃OD) § 8.39 (d, J=8.4 Hz, 1H), 8.19-8.11 (m, 3H), 7.83-7.81 (m, 1H), 7.71 (t, J=7.6 Hz, 1H), 7.34-7.30 (m, 2H), 7.20-7.18 (m, 1H), 3.72-3.68 (m, 4H), 3.59 (s, 2H), 3.13 (s, 2H), 2.39 (s, 3H), 1.75 (s, 2H), 1.34 (s, 2H). MS (TOF) [M+H]⁺=539.2.

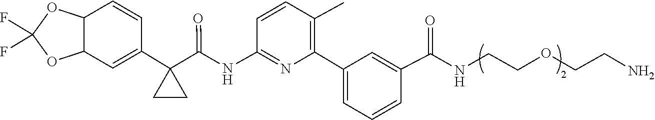

### Linker 94: N-(2-(2-(2-aminoethoxy)ethoxy)ethyl)-3-(6-(1-(2,2-difluorobenzo[d][1,3]dioxol-5-yl)cyclopropane-1-carboxamido)-3-methylpyridin-2-yl)benzamide

White solid, 60% yield. 1H NMR (400 MHz, CD₃OD) δ 8.08 (d, J=8.6 Hz, 1H), 7.84-7.77 (m, 3H), 7.52-7.50 (m, 1H), 7.41 (t, J=8.0 Hz, 1H), 7.04-7.00 (m, 2H), 6.91-6.88 (m, 1H), 3.39-3.36 (m, 8H), 3.28-3.27 (m, 2H), 2.77 (s, 2H), 2.09 (s, 3H), 1.45 (s, 2H), 1.05 (s, 2H). MS (TOF) [M+H]⁺=583.2.

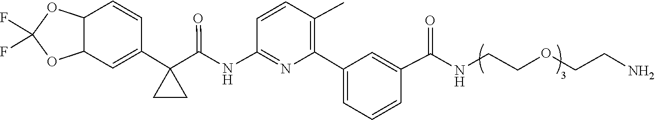

### Linker 95: N-(2-(2-(2-(2-aminoethoxy)ethoxy)ethoxy)ethyl)-3-(6-(1-(2,2-difluorobenzo[d][1,3]dioxol-5-yl)cyclopropane-1-carboxamido)-3-methylpyridin-2-yl)benzamide

White solid, 61% yield. ¹H NMR (400 MHz, CD₃OD) δ 7.70 (d, J=8.0 Hz, 1H), 7.59-7.50 (m, 3H), 7.22-7.19 (m 2H), 6.95-6.91 (m, 2H), 6.83-6.81 (m, 1H), 3.35-3.31 (m, 12H), 3.25-3.24 (m 2H), 2.78 (s, 2H), 1.93 (s, 3H), 1.31 (s, 2H), 0.88 (s, 2H). MS (TOF) [M+H]⁺=627.1.

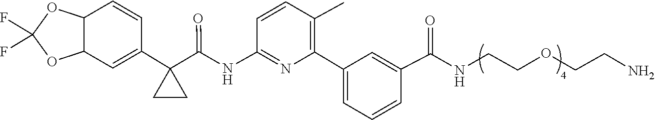

### Linker 96: N-(14-amino-3,6,9,12-tetraoxatetradecyl)-3-(6-(1-(2,2-difluorobenzo[d][1,3]dioxol-5-yl)cyclopropane-1-carboxamido)-3-methylpyridin-2-yl)benzamide

White solid, 58% yield. ¹H NMR (400 MHz, CD₃OD) δ 7.72 (d, J=8.0 Hz, 1H), 7.62-7.57 (m, 3H), 7.27-7.26 (m, 2H), 6.997-6.94 (m, 2H), 6.85-6.83 (m, 1H), 3.39-3.30 (m, 18H), 2.82 (s, 2H), 1.96 (s, 3H), 1.35 (s, 2H), 0.92 (s, 2H MS (TOF) [M+H]⁺=670.9.

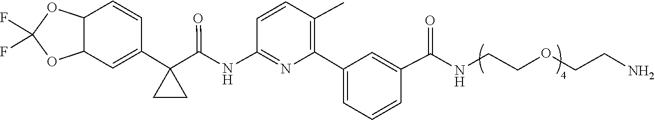

### Linker 97: N-(17-amino-3,6,9,12,15-pentaoxaheptadecyl)-3-(6-(1-(2,2-difluorobenzo[d][1,3]dioxol-5-yl)cyclopropane-1-carboxamido)-3-methylpyridin-2-yl)benzamide

White solid, 63% yield. ¹H NMR (400 MHz, CD₃OD) δ 7.85 (d, J=8.0 Hz, 1H), 7.73-7.63 (m, 3H), 7.37-7.33 (m, 2H), 7.11-7.06 (m, 2H), 6.98-6.96 (m, 1H), 3.52 (s, 2H), 3.45-3.40 (m, 20H), 2.94 (s, 2H), 2.06 (s, 3H), 1.46 (s, 2H), 1.03 (s, 2H). MS (TOF) [M+H]⁺=715.2.

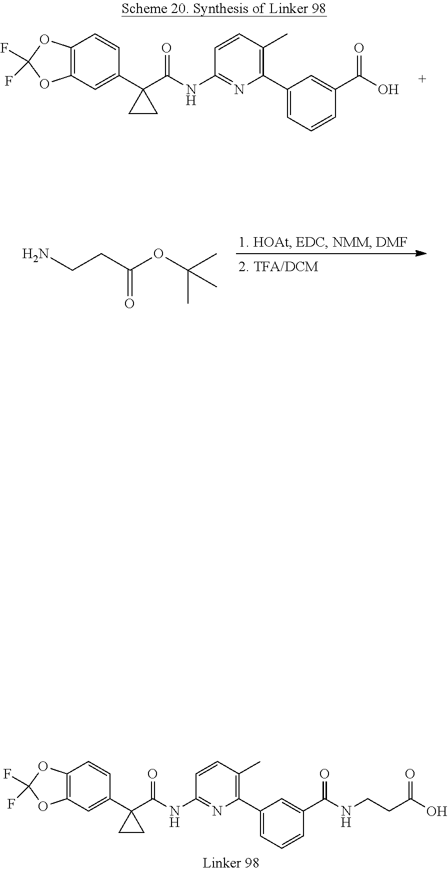

### Linker 98: 3-(3-(6-(1-(2,2-difluorobenzo[d][1,3]dioxol-5-yl)cyclopropane-1-carboxamido)-3-methylpyridin-2-yl)benzamido)propanoic acid

3-(6-(1-(2,2-difluorobenzo[d][1,3]dioxol-5-yl)cyclopropane-1-carboxamido)-3-methylpyridin-2-yl)benzoic acid (100 mg, 0.22 mmol, 1.0 eq), tert-butyl 3-aminopropanoate (31.5 mg, 0.24 mmol, 1.1 eq), EDC·HCl (63 mg, 0.33 mmol, 1.5 eq), HOAt (45 mg, 0.33 mmol, 1.5 eq), NMM (67 mg, 0.66 mmol, 3.0 eq) were stirred in 1.5 mL DMF at room temperature for 2 h. Then the mixture was purified via RP-C18 column [MeOH/Water (0.1% TFA)]. Then the product was dissolved in 1 mL DCM and 1 mL TFA, the mixture was stirred at room temperature for 1 h. Then removed all the volatiles and purified via RP-C18 column [MeOH/Water (0.1% TFA)]. The product was obtained as a white solid (70 mg, 61% yield over two steps). ¹H NMR (400 MHz, CD₃OD) δ 8.09-7.90 (m, 4H), 7.69-7.60 (m, 2H), 7.40 (s, 1H), 7.35 (d, J=8.4 Hz, 1H), 7.25 (d, J=8.3 Hz, 1H), 3.71-3.63 (m, 3H), 2.66 (t, J=7.3 Hz, 2H), 2.33 (s, 3H), 1.74 (s, 2H), 1.32 (s, 2H). MS (ESI) [M+H]⁺=524.3.

Linkers 99-110 are synthesized following the same procedure for preparing linker 98.

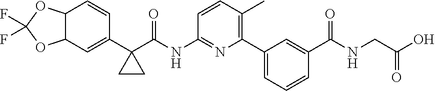 [SMILES: Cc1ccc(NC(=O)C2(C3=CC4OC(F)(F)OC4C=C3)CC2)nc1-c1cccc(C(=O)NCC(=O)O)c1]

### Linker 99: (3-(6-(1-(2,2-difluorobenzo[d][1,3]dioxol-5-yl)cyclopropane-1-carboxamido)-3-methylpyridin-2-yl)benzoyl)glycine

White solid, 62% yield. ¹H NMR (400 MHz, CD₃OD) δ 8.08 (d, J=8.5 Hz, 1H), 7.96 (d, J=6.6 Hz, 1H), 7.92 (s, 1H), 7.84 (d, J=8.5 Hz, 1H), 7.64-7.57 (dt, J=6.4, 1.6 Hz, 2H), 7.39 (s, 1H), 7.34 (d, J=8.3 Hz, 1H), 7.23 (d, J=8.3 Hz, 1H), 4.10 (s, 2H), 2.28 (s, 3H), 1.70 (s, 2H), 1.28 (s, 2H). MS (ESI) [M+H]⁺=510.3.

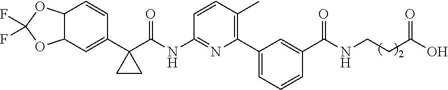

### Linker 100: 4-(3-(6-(1-(2,2-difluorobenzo[d][1,3]dioxol-5-yl)cyclopropane-1-carboxamido)-3-methylpyridin-2-yl)benzamido)butanoic acid

White solid, 63% yield. ¹H NMR (400 MHz, CD₃OD) δ 7.96 (d, J=8.5 Hz, 1H), 7.83-7.76 (m, 3H), 7.54-7.43 (m, 2H), 7.27 (s, 1H), 7.22 (d, J=8.3 Hz, 1H), 7.11 (d, J=8.3 Hz, 1H), 3.33 (t, J=6.9 Hz, 2H), 2.28 (t, J=7.3 Hz, 2H), 2.18 (s, 3H), 1.81 (p, J=7.2 Hz, 2H), 1.59 (s, 2H), 1.17 (s, 2H). MS (ESI) [M+H]⁺=538.3.

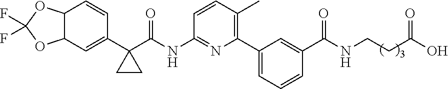

### Linker 101: 5-(3-(6-(1-(2,2-difluorobenzo[d][1,3]dioxol-5-yl)cyclopropane-1-carboxamido)-3-methylpyridin-2-yl)benzamido)pentanoic acid

White solid, 63% yield. ¹H NMR (400 MHz, CD₃OD) δ 8.08 (d, J=8.6 Hz, 1H), 7.94-7.84 (m, 3H), 7.62-7.57 (m, 2H), 7.40 (s, 1H), 7.34 (d, J=10.4 Hz, 1H), 7.24 (d, J=8.4 Hz, 1H), 3.42 (s, 2H), 2.37 (s, 2H), 2.29 (s, 3H), 1.73-1.63 (m, 6H), 1.29 (s, 2H). MS (ESI) [M+H]⁺=552.4.

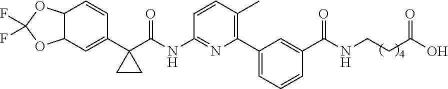

### Linker 102: 6-(3-(6-(1-(2,2-difluorobenzo[d][1,3]dioxol-5-yl)cyclopropane-1-carboxamido)-3-methylpyridin-2-yl)benzamido)hexanoic acid

White solid, 65% yield. ¹H NMR (400 MHz, CD₃OD) δ 8.06 (d, J=8.5 Hz, 1H), 7.88 (d, J=6.4 Hz, 1H), 7.85-7.80 (m, 2H), 7.56 (d, J=5.2 Hz, 2H), 7.37 (s, 1H), 7.31 (d, J=8.3 Hz, 1H), 7.21 (d, J=8.3 Hz, 1H), 3.38 (t, J=6.3 Hz, 2H), 2.31 (t, J=8.4 Hz, 2H), 2.27 (s, 3H), 1.71-1.58 (m, 6H), 1.48-1.37 (m, 2H), 1.26 (s, 2H). MS (ESI) [M+H]⁺=566.4.

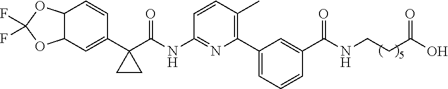

### Linker 103: 7-(3-(6-(1-(2,2-difluorobenzo[d][1,3]dioxol-5-yl)cyclopropane-1-carboxamido)-3-methylpyridin-2-yl)benzamido)heptanoic acid

White solid, 61% yield. ¹H NMR (400 MHz, CD₃OD) δ 8.05 (d, J=8.5 Hz, 1H), 7.87 (d, J=5.1 Hz, 1H), 7.85-7.79 (m, 2H), 7.59-7.51 (m, 2H), 7.35 (s, 1H), 7.30 (d, J=8.3 Hz, 1H), 7.19 (d, J=8.3 Hz, 1H), 3.38-3.33 (m, 2H), 2.31-2.24 (m, 5H), 1.66 (q, J=3.5 Hz, 2H), 1.64-1.56 (m, 4H), 1.39 (s, 4H), 1.24 (s, 2H). MS (ESI) [M+H]⁺=580.4.

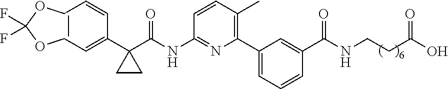

### Linker 104: 8-(3-(6-(1-(2,2-difluorobenzo[d][1,3]dioxol-5-yl)cyclopropane-1-carboxamido)-3-methylpyridin-2-yl)benzamido)octanoic acid

White solid, 59% yield. ¹H NMR (400 MHz, CD₃OD) δ 8.04 (d, J=8.5 Hz, 1H), 7.86 (d, J=6.0 Hz, 1H), 7.83 (s, 1H), 7.79 (d, J=8.5 Hz, 1H), 7.54 (d, J=5.0 Hz, 2H), 7.34 (s, 1H), 7.29 (d, J=8.1 Hz, 1H), 7.18 (d, J=8.3 Hz, 1H), 3.38-3.33 (m, 2H), 2.30-2.22 (m, 5H), 1.66 (s, 2H), 1.59 (s, 4H), 1.36 (s, 6H), 1.23 (s, 2H). MS (ESI) [M+H]⁺=594.4.

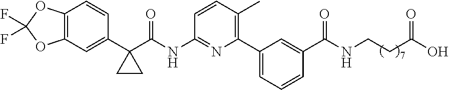

### Linker 105: 9-(3-(6-(1-(2,2-difluorobenzo[d][1,3]dioxol-5-yl)cyclopropane-1-carboxamido)-3-methylpyridin-2-yl)benzamido)nonanoic acid

White solid, 52% yield. MS (ESI) [M+H]⁺=608.5.

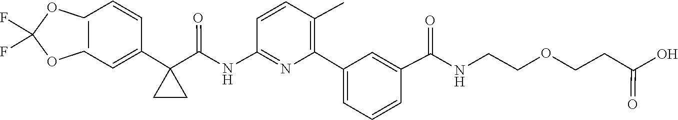

### Linker 106: 3-(2-(3-(6-(1-(2,2-difluorobenzo[d][1,3]dioxol-5-yl)cyclopropane-1-carboxamido)-3-methylpyridin-2-yl)benzamido)ethoxy)propanoic acid

White solid, 67% yield. ¹H NMR (400 MHz, CD₃OD) δ 8.03 (d, J=8.6 Hz, 1H), 7.88 (d, J=6.8 Hz, 1H), 7.83 (s, 1H), 7.78 (d, J=8.6 Hz, 1H), 7.57-7.49 (m, 2H), 7.32 (s, 1H), 7.28 (d, J=8.3 Hz, 1H), 7.17 (d, J=8.3 Hz, 1H), 3.71 (t, J=6.1 Hz, 2H), 3.64-3.57 (m, 2H), 3.56-3.50 (m, 2H), 3.29 (s, 1H), 2.52 (t, J=6.1 Hz, 2H), 2.24 (s, 3H), 1.65 (s, 2H), 1.22 (s, 2H). MS (ESI) [M+H]⁺=568.4.

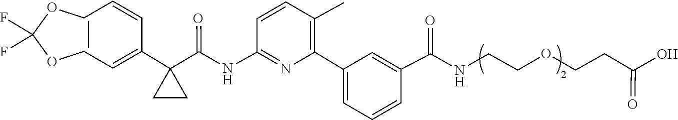

### Linker 107: 3-(2-(2-(3-(6-(1-(2,2-difluorobenzo[d][1,3]dioxol-5-yl)cyclopropane-1-carboxamido)-3-methylpyridin-2-yl)benzamido)ethoxy)ethoxy)propanoic acid

White solid, 63% yield. ¹H NMR (400 MHz, CD₃OD) δ 8.05 (d, J=8.8 Hz, 1H), 7.93-7.89 (m, 1H), 7.86 (s, 1H), 7.83 (d, J=7.9 Hz, 1H), 7.60-7.53 (m, 2H), 7.36 (s, 1H), 7.31 (d, J=8.2 Hz, 1H), 7.21 (d, J=8.3 Hz, 1H), 3.71 (d, J=5.5 Hz, 2H), 3.67-3.53 (m, 8H), 2.48 (t, J=5.8 Hz, 2H), 2.26 (s, 3H), 1.68 (s, 2H), 1.25 (s, 2H). MS (ESI) [M+H]⁺=612.4.

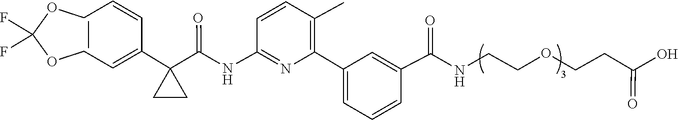

### Linker 108: 1-(3-(6-(1-(2,2-difluorobenzo[d][1,3]dioxol-5-yl)cyclopropane-1-carboxamido)-3-methylpyridin-2-yl)phenyl)-1-oxo-5,8,11-trioxa-2-azatetradecan-14-oic acid

White solid, 68% yield. ¹H NMR (400 MHz, CD₃OD) δ 8.04 (d, J=7.8 Hz, 1H), 7.95-7.85 (m, 3H), 7.63-7.54 (m, 2H), 7.36 (s, 1H), 7.30 (d, J=9.0 Hz, 1H), 7.20 (d, J=7.4 Hz, 1H), 3.67-3.50 (m, 14H), 2.47 (t, J=6.1 Hz, 2H), 2.27 (s, 3H), 1.68 (s, 2H), 1.26 (s, 2H). MS (ESI) [M+H]⁺=656.6.

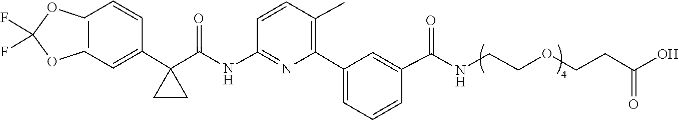

### Linker 109: 1-(3-(6-(1-(2,2-difluorobenzo[d][1,3]dioxol-5-yl)cyclopropane-1-carboxamido)-3-methylpyridin-2-yl)phenyl)-1-oxo-5,8,11,14-tetraoxa-2-azaheptadecan-17-oic acid

White solid, 65% yield. ¹H NMR (400 MHz, CD₃OD) δ 8.04-7.97 (m, 2H), 7.97-7.93 (m, 1H), 7.91 (s, 1H), 7.65-7.56 (m, 2H), 7.36 (s, 1H), 7.30 (d, J=7.9 Hz, 1H), 7.20 (d, J=8.1 Hz, 1H), 3.68-3.60 (m, 8H), 3.59-3.50 (m, 10H), 2.48 (t, J=6.1 Hz, 2H), 2.29 (s, 3H), 1.69 (s, 2H), 1.28 (s, 2H). MS (ESI) [M+H]⁺=700.5.

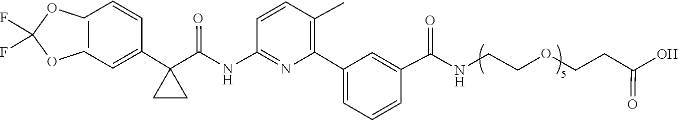

### Linker 110: 1-(3-(6-(1-(2,2-difluorobenzo[d][1,3]dioxol-5-yl)cyclopropane-1-carboxamido)-3-methylpyridin-2-yl)phenyl)-1-oxo-5,8,11,14,17-pentaoxa-2-azaicosan-20-oic acid

White solid, 61% yield. ¹H NMR (400 MHz, CD₃OD) δ 7.97 (s, 2H), 7.91 (d, J=6.9 Hz, 1H), 7.87 (s, 1H), 7.63-7.53 (m, 2H), 7.31 (s, 1H), 7.25 (d, J=8.2 Hz, 1H), 7.16 (d, J=8.3 Hz, 1H), 3.65-3.56 (m, 8H), 3.55-3.47 (m, 14H), 2.45 (t, J=6.0 Hz, 2H), 2.25 (s, 3H), 1.65 (s, 2H), 1.24 (s, 2H). MS (ESI) [M+H]⁺=744.5.

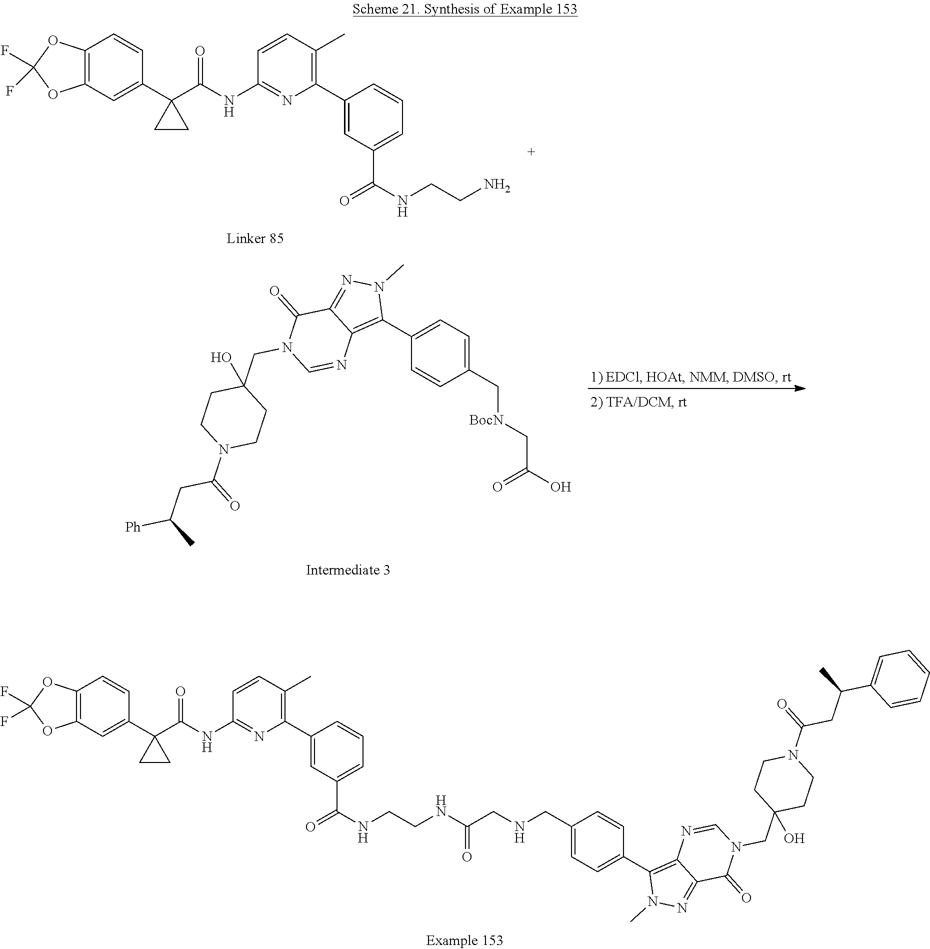

### Example 153: (R)-3-(6-(1-(2,2-difluorobenzo[d][1,3]dioxol-5-yl)cyclopropane-1-carboxamido)-3-methylpyridin-2-yl)-N-(2-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)ethyl)benzamide (XH168-51)

To a solution of linker 85 (10 mg, 0.013 mmol) in DMF (0.5 mL) were added intermediate 3 (2, 8.5 mg, 0.013 mmol, 1.0 equiv), 1-ethyl-3-(3-dimethylaminopropyl)carbodiimide (EDCI, 2.7 mg, 0.0195 mmol, 1.5 equiv), 1-hydroxy-7-azabenzo-triazole (HOAt, 3.7 mg, 0.0195 mmol, 1.5 equiv), and N-methylmorpholine (NMM, 3.9 mg, 0.039 mmol, 3.0 equiv). After being stirred for overnight at rt, the resulting mixture was purified by preparative HPLC to afford crude product. The crude product was dissolved in MeOH (0.5 mL) and HCl in dioxane (4 M, 0.5 mL). After being stirred at rt for 30 min, the resulting mixture was purified by preparative HPLC to afford titled compound as white solid (9.8 mg, yield 59%). ¹H NMR (400 MHz, Methanol-d₄) δ 9.09 (s, 1H), 8.38 (d, J=8.0 Hz, 1H), 8.14-8.11 (m, 3H), 7.81-7.79 (m, 3H), 7.72-7.70 (m, 3H), 7.39-7.20 (m, 8H), 4.37 (s, 2H), 4.25 (s, 1H), 4.09 (s, 4H), 3.89 (s, 2H), 3.72-3.69 (m, 1H), 3.64-3.60 (m, 1H), 3.56-3.53 (m, 2H), 3.46-3.45 (m, 2H), 3.26 (s, 3H), 3.22-3.20 (m, 1H), 3.12-3.01 (m, 2H), 2.80-2.76 (m, 1H), 2.37 (s, 3H), 1.74 (s, 2H), 1.58-1.55 (m, 1H), 1.40-1.34 (m, 6H). MS (ESI) [M+H]⁺=1049.3.

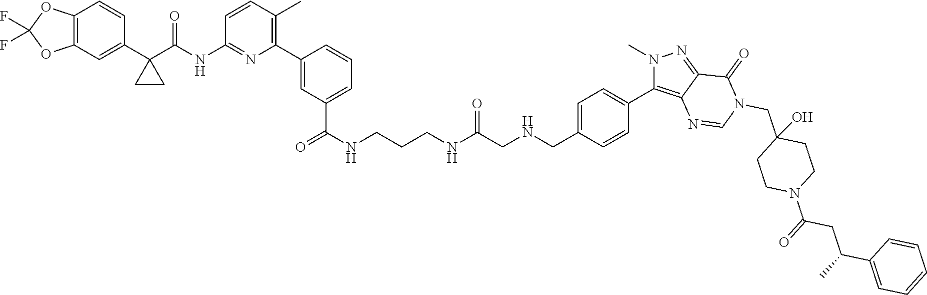 [SMILES: Cc1ccc(NC(=O)C2(c3ccc4c(c3)OC(F)(F)O4)CC2)nc1-c1cccc(C(=O)NCCCNC(=O)CNCc2ccc(-c3c4ncn(CC5(O)CCN(C(=O)C[C@@H](C)c6ccccc6)CC5)c(=O)c4nn3C)cc2)c1]

### Example 154: (R)-3-(6-(1-(2,2-difluorobenzo[d][1,3]dioxol-5-yl)cyclopropane-1-carboxamido)-3-methylpyridin-2-yl)-N-(3-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)propyl)benzamide (XH168-32)

Example 154 was synthesized following the same procedure for preparing example 153 from linker 84 and intermediate 3. (white solid, 9.3 mg, yield 55%). ¹H NMR (400 MHz, Methanol-d₄) δ 8.39 (s, 2H), 8.09-8.08 (m, 3H), 7.77-7.75 (m, 6H), 7.40-7.22 (m, 8H), 4.39 (s, 2H), 4.23-4.01 (m, 5H), 3.94-3.90 (m, 2H), 3.73-3.57 (m, 4H), 3.43 (s, 2H), 3.34 (s, 2H), 3.24-3.23 (m, 2H), 3.05-2.91 (m, 1H), 2.86-2.75 (m, 1H), 2.63-2.50 (m, 1H), 2.41 (s, 3H), 1.78 (s, 2H), 1.62-1.58 (m, 1H), 1.39-1.27 (m, 8H). MS (ESI) [M+H]⁺=1063.3.

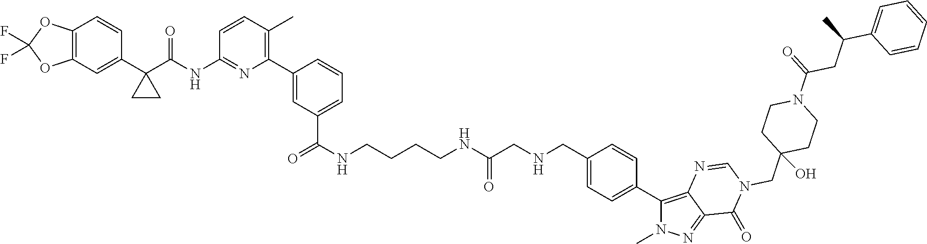 [SMILES: Cc1ccc(NC(=O)C2(c3ccc4c(c3)OC(F)(F)O4)CC2)nc1-c1cccc(C(=O)NCCCCNC(=O)CNCc2ccc(-c3c4ncn(CC5(O)CCN(C(=O)C[C@@H](C)c6ccccc6)CC5)c(=O)c4nn3C)cc2)c1]

### Example 155: (R)-3-(6-(1-(2,2-difluorobenzo[d][1,3]dioxol-5-yl)cyclopropane-1-carboxamido)-3-methylpyridin-2-yl)-N-(4-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)butyl)benzamide (XH168-33)

Example 155 was synthesized following the same procedure for preparing example 153 from linker 86 and intermediate 3. (white solid, 9.8 mg, yield 58%). ¹H NMR (400 MHz, Methanol-d₄) δ 8.39-8.35 (m, 2H), 8.08-8.06 (m, 3H), 7.80-7.72 (m, 6H), 7.40-7.16 (m, 8H), 4.38 (s, 2H), 4.23-4.11 (m, 5H), 4.05-3.87 (m, 3H), 3.73-3.57 (m, 3H), 3.41 (s, 2H), 3.34 (d, J=2.4 Hz, 2H), 3.24-3.21 (m, 2H), 3.02-2.91 (m, 1H), 2.85-2.75 (m, 1H), 2.63-2.49 (m, 1H), 2.40 (s, 3H), 1.78 (s, 2H), 1.68-1.61 (m, 4H), 1.39-1.32 (m, 8H). MS (ESI) [M+H]⁺=1077.4.

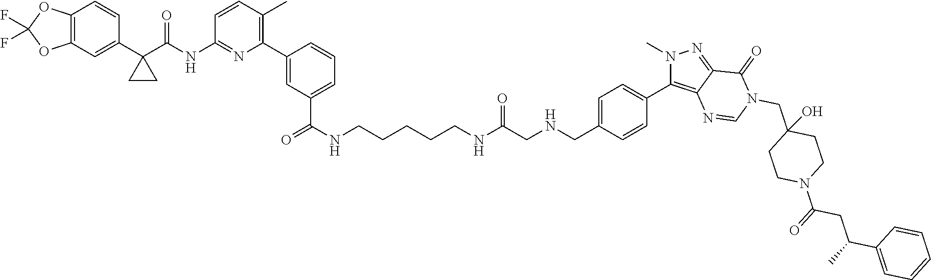 [SMILES: Cc1ccc(NC(=O)C2(c3ccc4c(c3)OC(F)(F)O4)CC2)nc1-c1cccc(C(=O)NCCCCCNC(=O)CNCc2ccc(-c3c4ncn(CC5(O)CCN(C(=O)C[C@@H](C)c6ccccc6)CC5)c(=O)c4nn3C)cc2)c1]

### Example 156: (R)-3-(6-(1-(2,2-difluorobenzo[d][1,3]dioxol-5-yl)cyclopropane-1-carboxamido)-3-methylpyridin-2-yl)-N-(5-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)pentyl)benzamide (XH168-59)

Example 156 was synthesized following the same procedure for preparing example 153 from linker 87 and intermediate 3. (white solid, 13.9 mg, yield 81%). ¹H NMR (400 MHz, Methanol-d₄) δ 8.49-8.36 (m, 2H), 8.03 (s, 3H), 7.74-7.69 (m, 6H), 7.36-7.18 (m, 8H), 4.35 (s, 2H), 4.18-3.99 (m, 5H), 3.92-3.83 (m, 3H), 3.69-3.53 (m, 3H), 3.35 (s, 2H), 3.26-3.23 (m, 3H), 2.99-2.92 (m, 1H), 2.80-2.74 (m, 1H), 2.60-2.51 (m, 1H), 2.38 (s, 3H), 1.75 (s, 2H), 1.62-1.55 (m, 4H), 1.41-1.24 (m, 10H). MS (ESI) [M+H]⁺=1091.4.

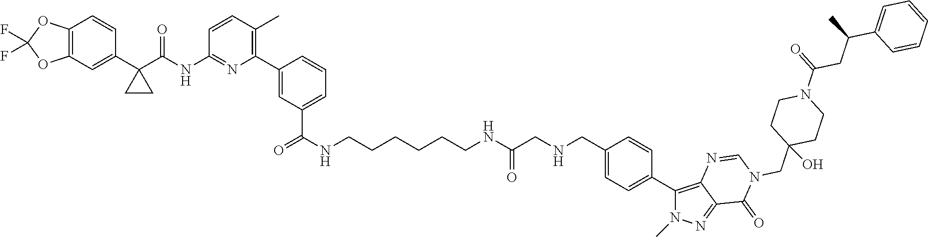 [SMILES: Cc1ccc(NC(=O)C2(c3ccc4c(c3)OC(F)(F)O4)CC2)nc1-c1cccc(C(=O)NCCCCCCNC(=O)CNCc2ccc(-c3c4ncn(CC5(O)CCN(C(=O)C[C@@H](C)c6ccccc6)CC5)c(=O)c4nn3C)cc2)c1]

### Example 157: (R)-3-(6-(1-(2,2-difluorobenzo[d][1,3]dioxol-5-yl)cyclopropane-1-carboxamido)-3-methylpyridin-2-yl)-N-(6-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)hexyl)benzamide (XH168-66)

Example 157 was synthesized following the same procedure for preparing example 153 from linker 88 and intermediate 3. (white solid, 9.8 mg, yield 59%). ¹H NMR (400 MHz, Methanol-d₄) δ 8.50-8.42 (m, 2H), 8.10 (s, 3H), 7.80-7.75 (m, 6H), 7.43-7.20 (m, 8H), 4.42 (s, 2H), 4.26-4.06 (s, 5H), 3.99-3.90 (s, 3H), 3.76-3.60 (m, 3H), 3.42-3.37 (m, 3H), 3.29 (s, 4H), 3.06-2.95 (m, 1H), 2.89-2.78 (m, 1H), 2.67-2.56 (m, 1H), 2.44 (s, 3H), 1.81 (s, 2H), 1.67 (s, 2H), 1.59 (s, 2H), 1.45-1.37 (m, 10H). MS (ESI) [M+H]⁺=1105.4.

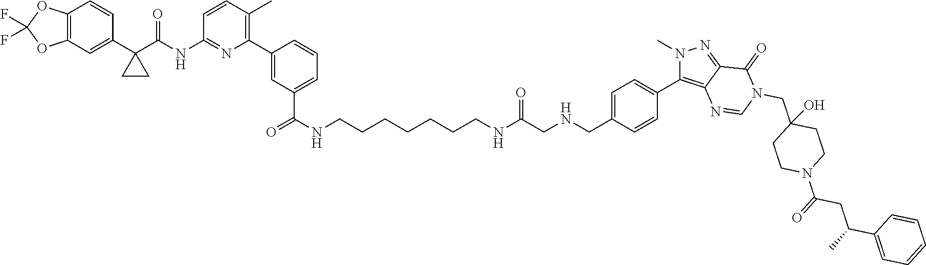 [SMILES: Cc1ccc(NC(=O)C2(c3ccc4c(c3)OC(F)(F)O4)CC2)nc1-c1cccc(C(=O)NCCCCCCCNC(=O)CNCc2ccc(-c3c4ncn(CC5(O)CCN(C(=O)C[C@@H](C)c6ccccc6)CC5)c(=O)c4nn3C)cc2)c1]

### Example 158: (R)-3-(6-(1-(2,2-difluorobenzo[d][1,3]dioxol-5-yl)cyclopropane-1-carboxamido)-3-methylpyridin-2-yl)-N-(7-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)heptyl)benzamide (XH168-67)

Example 158 was synthesized following the same procedure for preparing example 153 from linker 89 and intermediate 3. (white solid, 11.8 mg, yield 76%). ¹H NMR (400 MHz, Methanol-d₄) δ 8.44-8.38 (m, 2H), 8.07-8.06 (m, 3H), 7.80-7.70 (m, 6H), 7.40-7.16 (m, 8H), 4.38 (s, 2H), 4.23-4.18 (m, 5H), 4.05-3.86 (m, 3H), 3.78-3.57 (m, 3H), 3.40-3.34 (m, 3H), 3.26-3.23 (m, 4H), 3.06-2.92 (m, 1H), 2.87-2.74 (m, 1H), 2.63-2.50 (m, 1H), 2.40 (s, 3H), 1.78 (s, 2H), 1.63 (s, 3H), 1.54 (s, 2H), 1.48-1.28 (m, 12H). MS (ESI) [M+H]⁺=1119.5.

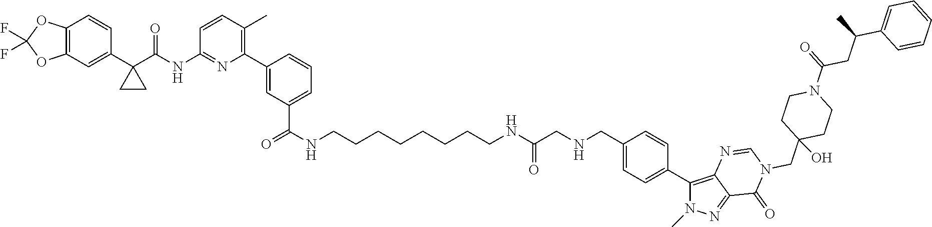 [SMILES: Cc1ccc(NC(=O)C2(c3ccc4c(c3)OC(F)(F)O4)CC2)nc1-c1cccc(C(=O)NCCCCCCCCNC(=O)CNCc2ccc(-c3c4ncn(CC5(O)CCN(C(=O)C[C@@H](C)c6ccccc6)CC5)c(=O)c4nn3C)cc2)c1]

### Example 133: (R)-3-(6-(1-(2,2-difluorobenzo[d][1,3]dioxol-5-yl)cyclopropane-1-carboxamido)-3-methylpyridin-2-yl)-N-(8-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)octyl)benzamide (XH168-68)

Example 159 was synthesized following the same procedure for preparing example 153 from linker 90 and intermediate 3. (white solid, 13.6 mg, yield 87%). ¹H NMR (400 MHz, Methanol-d₄) δ 8.84-8.81 (m, 1H), 8.44-8.42 (m, 1H), 8.11 (s, 3H), 7.84-7.76 (m, 6H), 7.44-7.23 (m, 8H), 4.43 (s, 2H), 4.29-4.05 (m, 6H), 3.91 (s, 2H), 3.78-3.60 (m, 4H), 3.41-3.38 (m, 3H), 3.27 (s, 3H), 3.09-3.03 (m, 1H), 2.95-2.85 (m, 1H), 2.72-2.61 (m, 1H), 2.44 (s, 3H), 1.82 (s, 2H), 1.66 (s, 2H), 1.57 (s, 2H), 1.42-1.31 (m, 14H). MS (ESI) [M+H]⁺=1133.4.

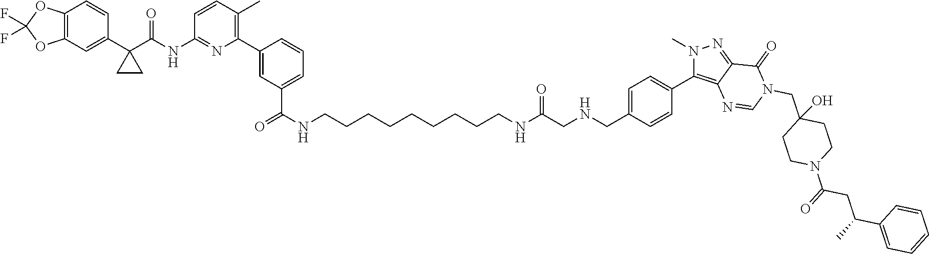 [SMILES: Cc1ccc(NC(=O)C2(c3ccc4c(c3)OC(F)(F)O4)CC2)nc1-c1cccc(C(=O)NCCCCCCCCCNC(=O)CNCc2ccc(-c3c4ncn(CC5(O)CCN(C(=O)C[C@@H](C)c6ccccc6)CC5)c(=O)c4nn3C)cc2)c1]

### Example 160: (R)-3-(6-(1-(2,2-difluorobenzo[d][1,3]dioxol-5-yl)cyclopropane-1-carboxamido)-3-methylpyridin-2-yl)-N-(9-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)nonyl)benzamide (XH168-69)

Example 160 was synthesized following the same procedure for preparing example 153 from linker 91 and intermediate 3. (white solid, 11.6 mg, yield 73%). ¹H NMR (400 MHz, Methanol-d₄) δ 8.66 (s, 1H), 8.39 (s, 1H), 8.06 (s, 3H), 7.77-7.72 (m, 6H), 7.39-7.18 (m, 8H), 4.38 (s, 2H), 4.19-3.96 (m, 6H), 3.86 (s, 2H), 3.72-3.58 (m, 4H), 3.37 (s, 3H), 3.23 (s, 3H), 3.04-2.97 (m, 1H), 2.85-2.78 (m, 1H), 2.65-2.54 (m, 1H), 2.40 (s, 3H), 1.78 (s, 2H), 1.62 (s, 2H), 1.51 (s, 2H), 1.36-1.27 (m, 16H). MS (ESI) [M+H]⁺=1147.2.

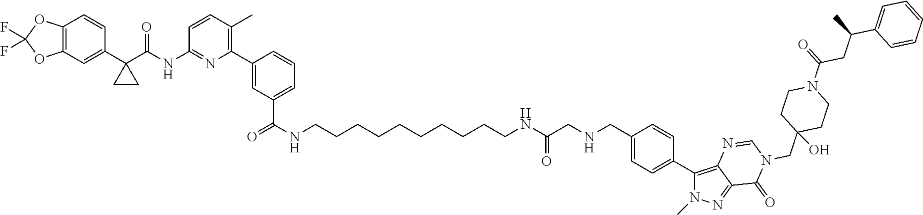 [SMILES: Cc1ccc(NC(=O)C2(c3ccc4c(c3)OC(F)(F)O4)CC2)nc1-c1cccc(C(=O)NCCCCCCCCCCNC(=O)CNCc2ccc(-c3c4ncn(CC5(O)CCN(C(=O)C[C@@H](C)c6ccccc6)CC5)c(=O)c4nn3C)cc2)c1]

### Example 161: (R)-3-(6-(1-(2,2-difluorobenzo[d][1,3]dioxol-5-yl)cyclopropane-1-carboxamido)-3-methylpyridin-2-yl)-N-(10-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)decyl)benzamide (XH168-70)

Example 161 was synthesized following the same procedure for preparing example 153 from linker 92 and intermediate 3. (white solid, 13.2 mg, yield 82%). ¹H NMR (400 MHz, Methanol-d₄) δ 8.50-8.46 (m, 1H), 8.40-8.38 (m, 1H), 8.07-8.05 (m, 3H), 7.80-7.73 (m, 6H), 7.40-7.16 (m, 8H), 4.38 (s, 2H), 4.23-3.93 (m, 6H), 3.86 (s, 2H), 3.73-3.57 (m, 4H), 3.40-3.34 (m, 4H), 3.25-3.22 (m, 3H), 3.06-2.95 (m, 1H), 2.87-2.76 (m, 1H), 2.64-2.51 (m, 1H), 2.41 (s, 3H), 1.78 (s, 2H), 1.62 (s, 2H), 1.52 (s, 2H), 1.39-1.32 (m, 18H). MS (ESI) [M+H]⁺=1161.8.

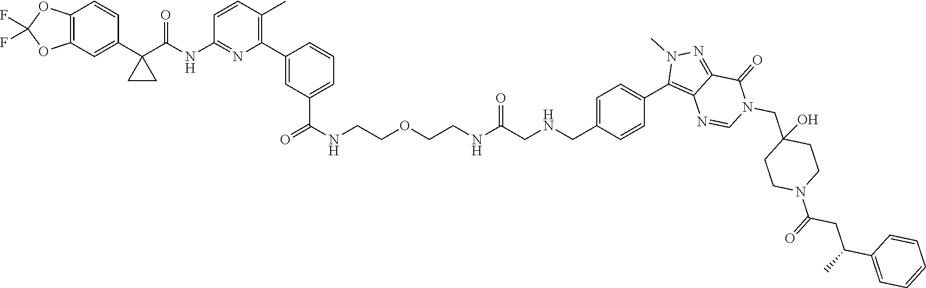 [SMILES: Cc1ccc(NC(=O)C2(c3ccc4c(c3)OC(F)(F)O4)CC2)nc1-c1cccc(C(=O)NCCOCCNC(=O)CNCc2ccc(-c3c4ncn(CC5(O)CCN(C(=O)C[C@@H](C)c6ccccc6)CC5)c(=O)c4nn3C)cc2)c1]

### Example 162: (R)-3-(6-(1-(2,2-difluorobenzo[d][1,3]dioxol-5-yl)cyclopropane-1-carboxamido)-3-methylpyridin-2-yl)-N-(2-(2-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)ethoxy)ethyl)benzamide (XH168-58)

Example 162 was synthesized following the same procedure for preparing example 153 from linker 93 and intermediate 3. (white solid, 14.1 mg, yield 93%). ¹H NMR (400 MHz, Methanol-d₄) δ 8.50-8.39 (m, 2H), 8.09 (m, 3H), 7.77-7.72 (m, 6H), 7.40-7.17 (m, 8H), 4.37 (s, 2H), 4.22-4.03 (m, 6H), 3.96-3.88 (m, 2H), 3.73-3.59 (m, 8H), 3.45 (s, 2H), 3.34 (s, 2H), 3.23 (s, 1H), 3.03-2.95 (m, 1H), 2.84-2.77 (m, 1H), 2.64-2.54 (m, 1H), 2.41 (s, 3H), 1.78 (s, 2H), 1.61 (s, 1H), 1.45-1.28 (m, 6H). MS (ESI) [M+H]⁺=1093.4.

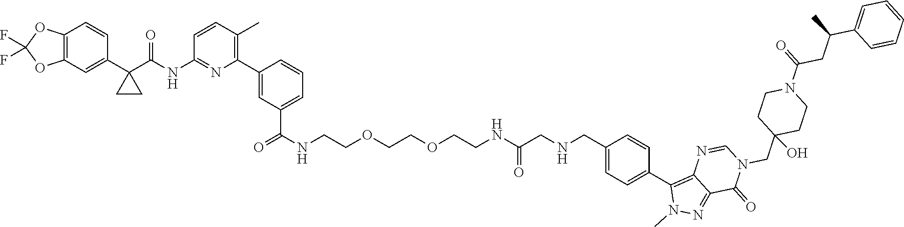 [SMILES: Cc1ccc(NC(=O)C2(c3ccc4c(c3)OC(F)(F)O4)CC2)nc1-c1cccc(C(=O)NCCOCCOCCNC(=O)CNCc2ccc(-c3c4ncn(CC5(O)CCN(C(=O)C[C@@H](C)c6ccccc6)CC5)c(=O)c4nn3C)cc2)c1]

### Example 163: (R)-3-(6-(1-(2,2-difluorobenzo[d][1,3]dioxol-5-yl)cyclopropane-1-carboxamido)-3-methylpyridin-2-yl)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11-dioxa-2,5-diazatridecan-13-yl)benzamide (XH168-47)

Example 163 was synthesized following the same procedure for preparing example 153 from linker 94 and intermediate 3. (white solid, 6.6 mg, yield 42%) ¹H NMR (400 MHz, Methanol-d₄) δ 8.38 (d, J=8.0 Hz, 1H), 8.33-8.29 (m, 1H), 8.07-8.05 (m, 3H), 7.80-7.72 (m, 6H), 7.40 (s, 1H), 7.34-7.16 (m, 7H), 4.38 (s, 2H), 4.22-4.00 (m, 6H), 3.93-3.86 (s, 2H), 3.73-3.65 (m, 8H), 3.56 (s, 4H), 3.42 (s, 2H), 3.34 (s, 2H), 3.25-3.23 (m, 1H), 3.04-2.90 (m, 1H), 2.85-2.73 (m, 1H), 2.63-2.49 (m, 1H), 2.40 (s, 3H), 1.78 (s, 2H), 1.63-1.55 (m, 1H), 1.39-1.28 (m, 6H). MS (ESI) [M+H]⁺=1137.5.

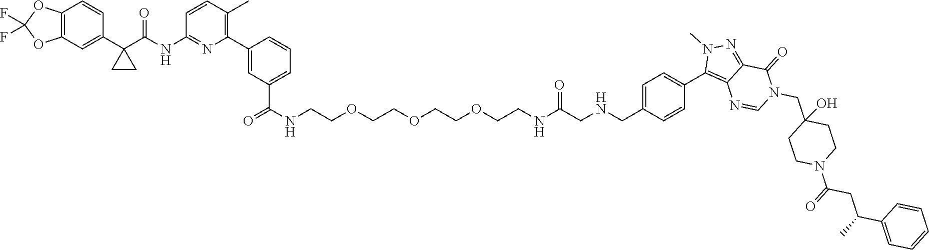 [SMILES: Cc1ccc(NC(=O)C2(c3ccc4c(c3)OC(F)(F)O4)CC2)nc1-c1cccc(C(=O)NCCOCCOCCOCCNC(=O)CNCc2ccc(-c3c4ncn(CC5(O)CCN(C(=O)C[C@@H](C)c6ccccc6)CC5)c(=O)c4nn3C)cc2)c1]

### Example 164: (R)-3-(6-(1-(2,2-difluorobenzo[d][1,3]dioxol-5-yl)cyclopropane-1-carboxamido)-3-methylpyridin-2-yl)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11,14-trioxa-2,5-diazahexadecan-16-yl)benzamide (XH168-48)

Example 164 was synthesized following the same procedure for preparing example 153 from linker 95 and intermediate 3. (white solid, 15.1 mg, yield 92%). ¹H NMR (400 MHz, Methanol-d₄) δ 8.67 (s, 1H), 8.40 (s, 1H), 8.08 (s, 3H), 7.80-7.73 (m, 6H), 7.40-7.16 (m, 8H), 4.40 (s, 2H), 4.20-4.13 (m, 5H), 3.88 (s, 3H), 3.63-3.57 (m, 16H), 3.41 (s, 2H), 3.34 (s, 3H), 3.24 (s, 1H), 3.05-2.98 (m, 1H), 2.85-2.81 (m, 1H), 2.66-2.56 (m, 1H), 2.42 (s, 3H), 1.78 (s, 2H), 1.50-1.35 (m, 7H). MS (ESI) [M+H]⁺=1181.4.

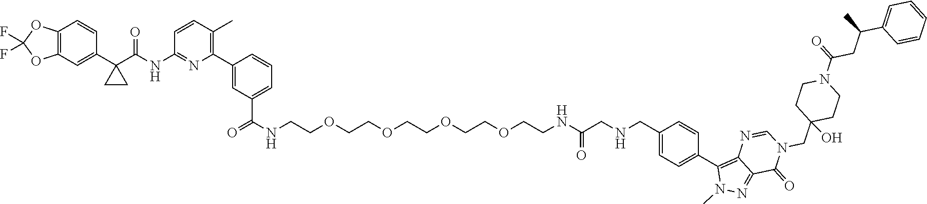 [SMILES: Cc1ccc(NC(=O)C2(c3ccc4c(c3)OC(F)(F)O4)CC2)nc1-c1cccc(C(=O)NCCOCCOCCOCCOCCNC(=O)CNCc2ccc(-c3c4ncn(CC5(O)CCN(C(=O)C[C@@H](C)c6ccccc6)CC5)c(=O)c4nn3C)cc2)c1]

### Example 165: (R)-3-(6-(1-(2,2-difluorobenzo[d][1,3]dioxol-5-yl)cyclopropane-1-carboxamido)-3-methylpyridin-2-yl)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11,14,17-tetraoxa-2,5-diazanonadecan-19-yl)benzamide (XH168-49)

Example 165 was synthesized following the same procedure for preparing example 153 from linker 96 and intermediate 3. (white solid, 15.7 mg, yield 93%). ¹H NMR (400 MHz, Methanol-d₄) δ 8.52-8.40 (m, 2H), 8.07 (s, 3H), 7.78-7.74 (m, 6H), 7.40-7.17 (m, 8H), 4.39 (s, 2H), 4.23-4.03 (m, 5H), 3.96-3.88 (m, 3H), 3.73-3.58 (m, 20H), 3.43 (s, 2H), 3.34 (m, 2H), 3.23 (s, 1H), 3.03-2.95 (m, 1H), 2.87-2.76 (m, 1H), 2.64-2.53 (m, 1H), 2.41 (s, 3H), 1.78 (s, 2H), 1.60 (s, 1H), 1.39-1.28 (m, 6H). MS (ESI) [M+H]⁺=1225.4.

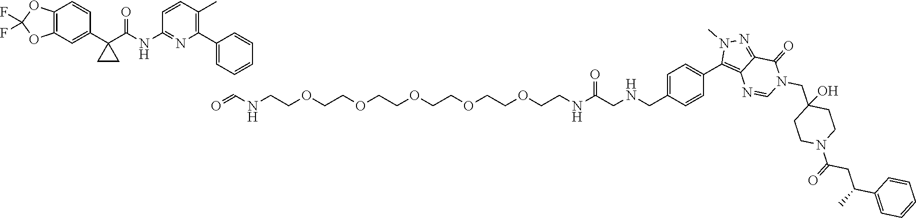

### Example 166: (R)-3-(6-(1-(2,2-difluorobenzo[d][1,3]dioxol-5-yl)cyclopropane-1-carboxamido)-3-methylpyridin-2-yl)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11,14,17,20-pentaoxa-2,5-diazadocosan-22-yl)benzamide (XH168-50)

Example 166 was synthesized following the same procedure for preparing example 153 from linker 97 and intermediate 3. (white solid, 16.1 mg, yield 92%) ¹H NMR (400 MHz, Methanol-d₄) δ 8.07-7.97 (m, 2H), 7.89-7.84 (m, 2H), 7.78-7.68 (m, 5H), 7.58-7.52 (m, 2H), 7.36-7.14 (m, 8H), 4.35 (s, 2H), 4.22-4.12 (m, 4H), 4.08-3.99 (m, 1H), 3.96-3.89 (m, 1H), 3.84 (s, 2H), 3.62-3.55 (m, 22H), 3.43-3.42 (m, 2H), 3.25-3.15 (m, 3H), 3.05-2.89 (m, 1H), 2.81-2.69 (m, 1H), 2.61-2.45 (m, 1H), 2.23 (s, 3H), 1.67 (s, 2H), 1.62-1.39 (m, 2H), 1.34-1.24 (m, 6H). MS (ESI) [M+H]⁺=1269.5.

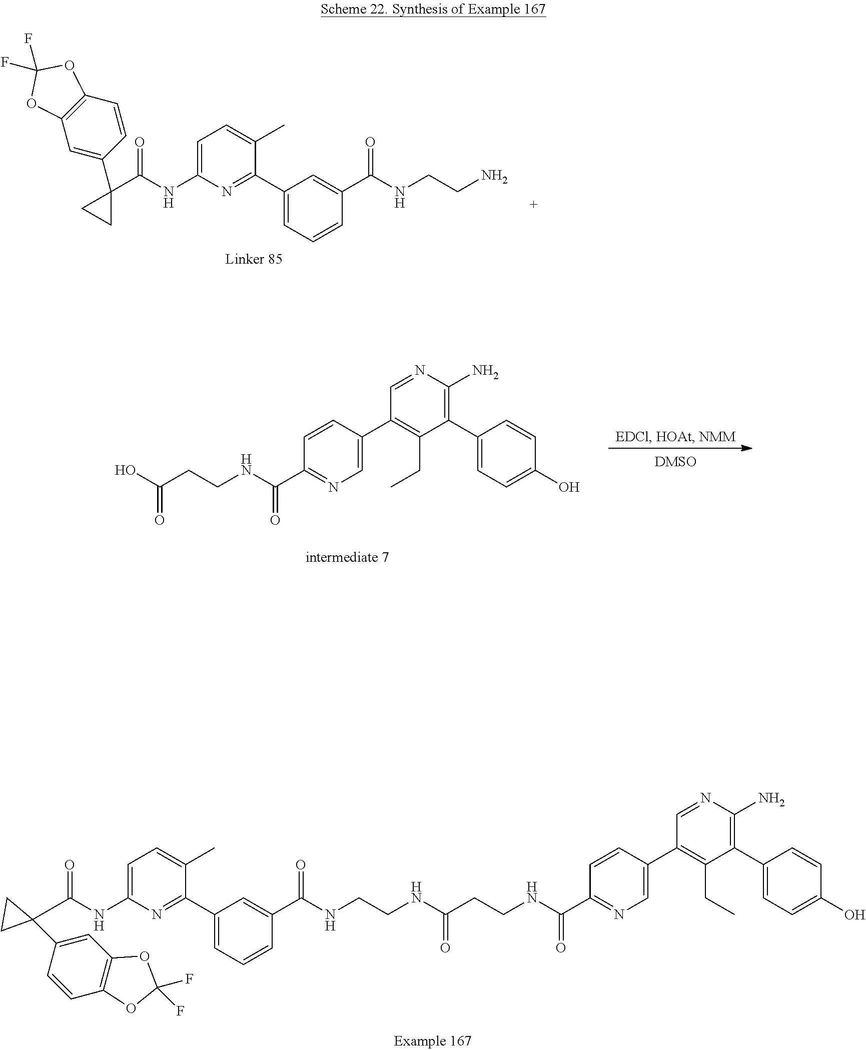 [SMILES: Cc1ccc(NC(=O)C2(c3ccc4c(c3)OC(F)(F)O4)CC2)nc1-c1cccc(C(=O)NCCN)c1 ; CCc1c(-c2ccc(C(=O)NCCC(=O)O)nc2)cnc(N)c1-c1ccc(O)cc1 ; CCc1c(-c2ccc(C(=O)NCCC(=O)NCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 167: 6′-amino-N-(3-((2-(3-(6-(1-(2,2-difluorobenzo[d][1,3]dioxol-5-yl)cyclopropane-1-carboxamido)-3-methylpyridin-2-yl)benzamido)ethyl)amino)-3-oxopropyl)-4′-ethyl-5′-(4-hydroxyphenyl)-[3,3′-bipyridine]-6-carboxamide (KL180-124)

To a solution of intermediate 7 (4 mg, 0.01 mmol) in DMSO (1 mL) were added linker 85 (4.9 mg, 0.01 mmol, 1.0 equiv), EDCI (1-ethyl-3-(3-dimethylaminopropyl)carbodiimide) (3.0 mg, 0.015 mmol, 1.5 equiv), HOAt (1-hydroxy-7-azabenzo-triazole) (2.0 mg, 0.015 mmol, 1.5 equiv), and NMM (N-Methylmorpholine) (3.0 mg, 0.03 mmol, 3.0 equiv). After being stirred overnight at room temperature, the resulting mixture was purified by preparative HPLC (5%-70% acetonitrile/0.1% TFA in H₂O) to afford titled compound as white solid (4.8 mg, 55%). ¹H NMR (400 MHz, Methanol-d₄) δ 8.63 (d, J=3.4 Hz, 1H), 8.20-8.12 (m, 1H), 8.11-8.05 (m, 1H), 8.05-7.98 (m, 1H), 7.94-7.85 (m, 2H), 7.83-7.76 (m, 2H), 7.62-7.52 (m, 2H), 7.39 (t, J=2.2 Hz, 1H), 7.37-7.31 (m, 1H), 7.28-7.15 (m, 3H), 7.09-6.97 (m, 2H), 3.71 (q, J=6.2, 4.6 Hz, 2H), 3.51 (q, J=5.4, 4.4 Hz, 2H), 3.44 (q, J=6.0, 5.2 Hz, 2H), 2.63-2.50 (m, 2H), 2.46-2.40 (m, 2H), 2.27 (t, J=2.6 Hz, 3H), 1.71-1.67 (m, 2H), 1.32 (t, J=2.6 Hz, 1H), 1.29-1.25 (m, 2H), 0.72 (q, J=7.0 Hz, 3H). MS (ESI) m/z 883.5 [M+H]⁺.

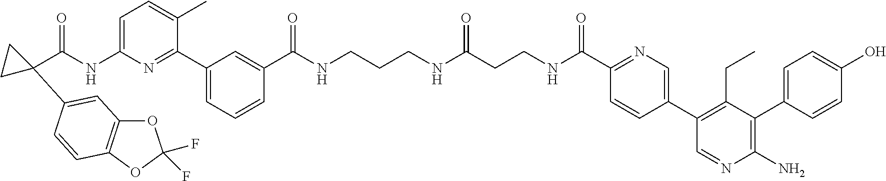 [SMILES: CCc1c(-c2ccc(C(=O)NCCC(=O)NCCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 168: 6′-amino-N-(3-((3-(3-(6-(1-(2,2-difluorobenzo[d][1,3]dioxol-5-yl)cyclopropane-1-carboxamido)-3-methylpyridin-2-yl)benzamido)propyl)amino)-3-oxopropyl)-4′-ethyl-5′-(4-hydroxyphenyl)-[3,3′-bipyridine]-6-carboxamide (KL180-125)

Example 168 was synthesized following similar procedure for preparing example 167 from linker 84 and intermediate 7. White solid, 5.0 mg, 54% yield. ¹H NMR (400 MHz, Methanol-d₄) δ 8.63 (d, J=3.3 Hz, 1H), 8.26-8.17 (m, 1H), 8.11-8.05 (m, 1H), 8.04-8.01 (m, 1H), 7.98-7.85 (m, 2H), 7.84-7.75 (m, 2H), 7.58 (dd, J=7.5, 4.3 Hz, 2H), 7.39 (d, J=3.4 Hz, 1H), 7.34 (dd, J=8.6, 3.1 Hz, 1H), 7.23 (dd, J=6.6, 4.0 Hz, 1H), 7.19-7.16 (dt, J=8.8, 2.1 Hz, 2H), 7.02 (dt, J=8.7, 2.3 Hz, 2H), 3.82-3.69 (m, 2H), 3.40 (q, J=4.1, 3.4 Hz, 2H), 2.66-2.55 (m, 2H), 2.44-2.38 (m, 2H), 2.28 (t, J=2.6 Hz, 3H), 1.81-1.75 (m, 2H), 1.72-1.68 (m, 2H), 1.32 (t, J=2.6 Hz, 2H), 1.29-1.25 m, 2H), 0.69 (d, J=9.3 Hz, 3H). MS (ESI) m/z 897.5 [M+H]⁺.

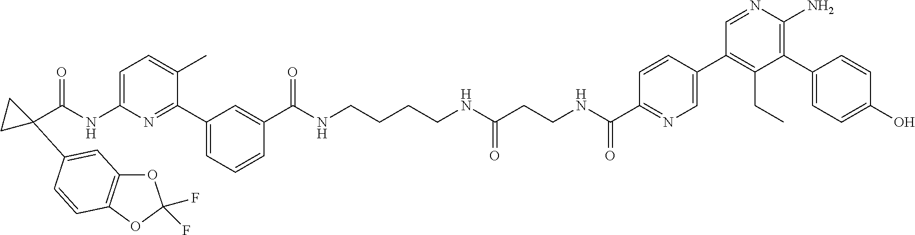 [SMILES: CCc1c(-c2ccc(C(=O)NCCC(=O)NCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 169: 6′-amino-N-(3-((4-(3-(6-(1-(2,2-difluorobenzo[d][1,3]dioxol-5-yl)cyclopropane-1-carboxamido)-3-methylpyridin-2-yl)benzamido)butyl)amino)-3-oxopropyl)-4′-ethyl-5′-(4-hydroxyphenyl)-[3,3′-bipyridine]-6-carboxamide (KL180-126)

Example 169 was synthesized following similar procedure for preparing example 167 from linker 86 and intermediate 7. White solid, 5.5 mg, 61% yield. ¹H NMR (400 MHz, Methanol-d₄) δ 8.66 (d, J=3.6 Hz, 1H), 8.26-8.17 (m, 1H), 8.08 (dt, J=8.6, 2.6 Hz, 1H), 8.06-8.00 (m, 1H), 7.94-7.84 (m, 2H), 7.82 (t, J=2.9 Hz, 2H), 7.57 (q, J=5.8, 4.4 Hz, 2H), 7.39 (d, J=3.5 Hz, 1H), 7.34 (dd, J=8.7, 3.2 Hz, 1H), 7.26-7.21 (m, 1H), 7.19 (dt, J=8.6, 2.2 Hz, 2H), 7.03 (dt, J=8.8, 2.3 Hz, 2H), 3.79-3.67 (m, 2H), 3.40-3.37 (m, 1H), 3.26 (d, J=16.1 Hz, 2H), 2.59-2.54 (m, 2H), 2.50-2.37 (m, 2H), 2.27 (t, J=2.6 Hz, 3H), 1.70 (q, J=3.0 Hz, 2H), 1.67-1.54 (m, 4H), 1.32 (t, J=2.6 Hz, 1H), 1.29-1.25 (m, 2H), 0.73-0.69 (m, 3H). MS (ESI) m/z 911.5 [M+H]⁺.

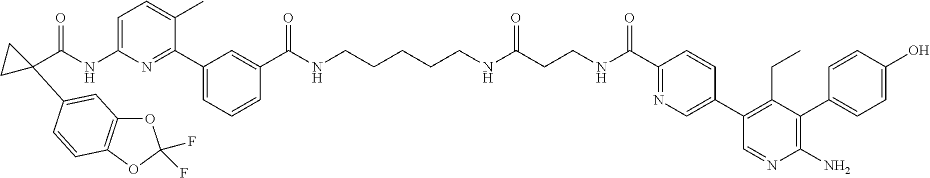 [SMILES: CCc1c(-c2ccc(C(=O)NCCC(=O)NCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 170: 6′-amino-N-(3-((5-(3-(6-(1-(2,2-difluorobenzo[d][1,3]dioxol-5-yl)cyclopropane-1-carboxamido)-3-methylpyridin-2-yl)benzamido)pentyl)amino)-3-oxopropyl)-4′-ethyl-5′-(4-hydroxyphenyl)-[3,3′-bipyridine]-6-carboxamide (KL180-127)

Example 170 was synthesized following similar procedure for preparing example 167 from linker 87 and intermediate 7. White solid, 4.9 mg, 53% yield. ¹H NMR (400 MHz, Methanol-d₄) δ 8.69 (d, J=3.3 Hz, 1H), 8.25-8.16 (m, 1H), 8.10-8.04 (m, 2H), 7.92-7.84 (m, 2H), 7.82 (q, J=5.1, 3.6 Hz, 2H), 7.58 (d, J=3.9 Hz, 2H), 7.40 (d, J=3.4 Hz, 1H), 7.34 (dd, J=8.7, 3.0 Hz, 1H), 7.26-7.20 (m, 1H), 7.18 (dt, J=8.6, 2.2 Hz, 2H), 7.02 (dt, J=8.5, 2.4 Hz, 2H), 3.72-3.68 (m, 2H), 3.37 (d, J=6.3 Hz, 2H), 3.23 (d, J=16.6 Hz, 2H), 2.53 (q, J=5.3, 4.3 Hz, 2H), 2.43 (dt, J=9.6, 5.0 Hz, 2H), 2.32-2.23 (m, 3H), 1.72-1.69 (m, 2H), 1.63 (d, J=8.5 Hz, 2H), 1.56 (t, J=8.5 Hz, 2H), 1.46-1.36 (m, 2H), 1.30-1.26 (m, 2H), 0.72 (q, J=6.8, 6.2 Hz, 3H). MS (ESI) m/z 925.6 [M+H]⁺.

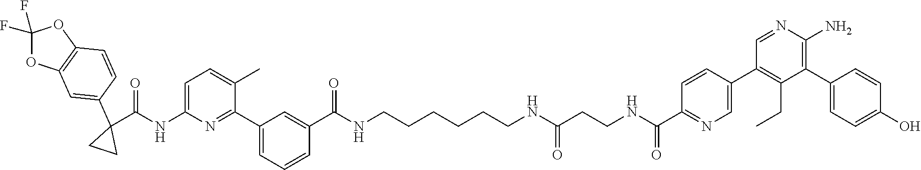 [SMILES: CCc1c(-c2ccc(C(=O)NCCC(=O)NCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 171: 6′-amino-N-(3-((6-(3-(6-(1-(2,2-difluorobenzo[d][1,3]dioxol-5-yl)cyclopropane-1-carboxamido)-3-methylpyridin-2-yl)benzamido)hexyl)amino)-3-oxopropyl)-4′-ethyl-5′-(4-hydroxyphenyl)-[3,3′-bipyridine]-6-carboxamide (KL180-128)

Example 171 was synthesized following similar procedure for preparing example 167 from linker 88 and intermediate 7. White solid, 5.3 mg, 57% yield. ¹H NMR (400 MHz, Methanol-d₄) δ 8.68 (d, J=3.4 Hz, 1H), 8.22 (q, J=2.8 Hz, 1H), 8.10-8.03 (m, 2H), 7.93-7.84 (m, 2H), 7.84-7.77 (m, 2H), 7.57 (q, J=5.6, 4.1 Hz, 2H), 7.42-7.36 (m, 1H), 7.34 (dd, J=8.9, 3.0 Hz, 1H), 7.23 (d, J=7.7 Hz, 1H), 7.18 (dd, J=7.2, 4.3 Hz, 2H), 7.02 (dt, J=8.8, 2.3 Hz, 2H), 3.74-3.70 (m, 2H), 3.38 (dd, J=6.4, 3.1 Hz, 1H), 3.24-3.19 (m, 2H), 2.58-2.53 (m, 2H), 2.48-2.41 (m, 2H), 2.27 (t, J=2.6 Hz, 3H), 1.72-1.68 (m, 2H), 1.61 (d, J=8.8 Hz, 2H), 1.56-1.48 (m, 2H), 1.45-1.35 (m, 4H), 1.32 (t, J=2.7 Hz, 1H), 1.29-1.25 (m, 2H), 0.72 (q, J=7.0, 5.8 Hz, 3H). MS (ESI) m/z 940.0 [M+H]⁺.

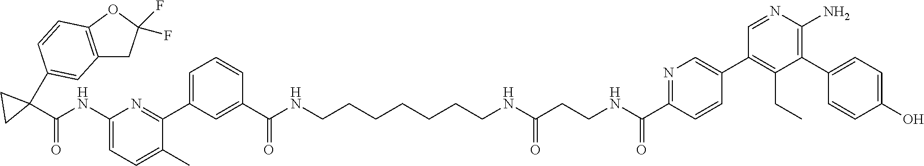 [SMILES: CCc1c(-c2ccc(C(=O)NCCC(=O)NCCCCCCCNC(=O)c3cccc(-c4nc(NC(=O)C5(c6ccc7c(c6)CC(F)(F)O7)CC5)ccc4C)c3)nc2)cnc(N)c1-c1ccc(O)cc1]

### Example 172: 6′-amino-N-(3-((7-(3-(6-(1-(2,2-difluorobenzo[d][1,3]dioxol-5-yl)cyclopropane-1-carboxamido)-3-methylpyridin-2-yl)benzamido)heptyl)amino)-3-oxopropyl)-4′-ethyl-5′-(4-hydroxyphenyl)-[3,3′-bipyridine]-6-carboxamide (KL180-129)

Example 172 was synthesized following similar procedure for preparing example 167 from linker 89 and intermediate 7. White solid, 4.5 mg, 47% yield. ¹H NMR (400 MHz, Methanol-d₄) δ 8.69 (d, J=3.5 Hz, 1H), 8.27-8.18 (m, 1H), 8.12-8.02 (m, 2H), 7.93-7.84 (m, 2H), 7.82 (t, J=2.9 Hz, 2H), 7.58 (q, J=5.6, 4.0 Hz, 2H), 7.40 (d, J=3.4 Hz, 1H), 7.35 (dd, J=8.7, 3.1 Hz, 1H), 7.25-7.21 (m, 1H), 7.18 (t, J=5.2 Hz, 2H), 7.02 (dt, J=7.9, 2.5 Hz, 2H), 3.79-3.64 (m, 2H), 3.39-3.36 (m, 1H), 3.20 (d, J=17.0 Hz, 2H), 2.56 (q, J=4.4, 3.6 Hz, 2H), 2.52-2.40 (m, 2H), 2.28 (t, J=2.6 Hz, 3H), 1.70 (q, J=2.9 Hz, 2H), 1.61 (s, 2H), 1.51 (s, 2H), 1.34 (d, J=16.6 Hz, 7H), 1.28 (q, J=2.9 Hz, 2H), 0.72 (q, J=5.9, 4.8 Hz, 3H). MS (ESI) m/z 953.5 [M+H]⁺.

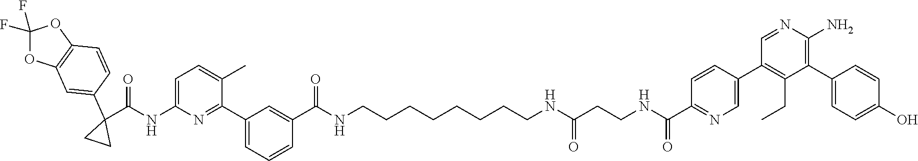 [SMILES: CCc1c(-c2ccc(C(=O)NCCC(=O)NCCCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 173: 6′-amino-N-(3-((8-(3-(6-(1-(2,2-difluorobenzo[d][1,3]dioxol-5-yl)cyclopropane-1-carboxamido)-3-methylpyridin-2-yl)benzamido)octyl)amino)-3-oxopropyl)-4′-ethyl-5′-(4-hydroxyphenyl)-[3,3′-bipyridine]-6-carboxamide (KL180-130)

Example 173 was synthesized following similar procedure for preparing example 167 from linker 90 and intermediate 7. White solid, 6.1 mg, 63% yield. ¹H NMR (400 MHz, Methanol-d₄) § 8.69 (d, J=3.4 Hz, 1H), 8.27-8.18 (m, 1H), 8.08 (td, J=10.5, 2.8 Hz, 2H), 7.93-7.87 (m, 1H), 7.88-7.85 (m, 1H), 7.85-7.79 (m, 2H), 7.58 (q, J=5.6, 4.1 Hz, 2H), 7.42-7.38 (m, 1H), 7.35 (dd, J=8.9, 3.1 Hz, 1H), 7.24 (d, J=8.1 Hz, 1H), 7.25-7.18 (m, 2H), 7.03 (dt, J=8.9, 2.4 Hz, 2H), 3.74-3.70 (m, 2H), 3.39 (d, J=8.8 Hz, 1H), 3.20 (q, J=8.4, 6.9 Hz, 2H), 2.57-2.53 (m, 2H), 2.48-2.42 (m, 2H), 2.28 (t, J=2.5 Hz, 3H), 1.73-1.68 (m, 2H), 1.66-1.55 (m, 2H), 1.50 (d, J=9.1 Hz, 2H), 1.43-1.30 (m, 10H), 1.28 (d, J=2.9 Hz, 1H), 0.80-0.68 (m, 3H). MS (ESI) m/z 967.5 [M+H]⁺.

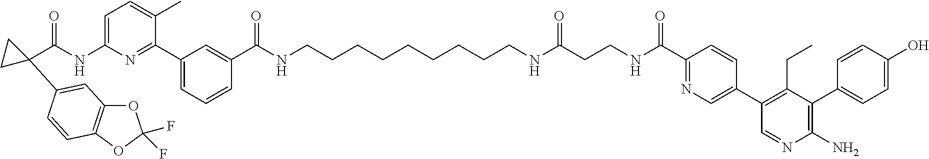 [SMILES: CCc1c(-c2ccc(C(=O)NCCC(=O)NCCCCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 174: 6′-amino-N-(3-((9-(3-(6-(1-(2,2-difluorobenzo[d][1,3]dioxol-5-yl)cyclopropane-1-carboxamido)-3-methylpyridin-2-yl)benzamido)nonyl)amino)-3-oxopropyl)-4′-ethyl-5′-(4-hydroxyphenyl)-[3,3′-bipyridine]-6-carboxamide (KL180-131)

Example 174 was synthesized following similar procedure for preparing example 167 from linker 91 and intermediate 7. White solid, 5.1 mg, 52% yield. ¹H NMR (400 MHz, Methanol-d₄) δ 8.69 (d, J=3.4 Hz, 1H), 8.25-8.18 (m, 1H), 8.11-8.04 (m, 2H), 7.94-7.88 (m, 1H), 7.86 (d, J=2.8 Hz, 1H), 7.82 (q, J=3.6, 2.5 Hz, 2H), 7.58 (q, J=5.6, 4.1 Hz, 2H), 7.40 (d, J=3.3 Hz, 1H), 7.35 (dd, J=8.8, 3.0 Hz, 1H), 7.25-7.18 (m, 3H), 7.03 (dt, J=8.8, 2.2 Hz, 2H), 3.75-3.70 (m, 2H), 3.39 (d, J=8.7 Hz, 2H), 3.19 (q, J=6.8, 5.1 Hz, 2H), 2.57-2.53 (m, 2H), 2.50-2.40 (m, 2H), 2.28 (t, J=2.7 Hz, 3H), 1.72-1.68 (m, 2H), 1.62 (t, J=8.0 Hz, 2H), 1.49 (t, J=7.5 Hz, 2H), 1.36 (s, 4H), 1.32 (d, J=3.7 Hz, 6H), 1.28 (q, J=2.9 Hz, 2H), 0.80-0.63 (m, 3H). MS (ESI) m/z 981.3 [M+H]⁺.

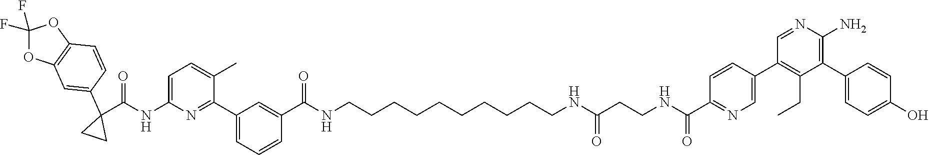 [SMILES: CCc1c(-c2ccc(C(=O)NCCC(=O)NCCCCCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 175: 6′-amino-N-(3-((10-(3-(6-(1-(2,2-difluorobenzo[d][1,3]dioxol-5-yl)cyclopropane-1-carboxamido)-3-methylpyridin-2-yl)benzamido)decyl)amino)-3-oxopropyl)-4′-ethyl-5′-(4-hydroxyphenyl)-[3,3′-bipyridine]-6-carboxamide (KL180-132)

Example 175 was synthesized following similar procedure for preparing example 167 from linker 92 and intermediate 7. White solid, 5.6 mg, 56% yield. ¹H NMR (400 MHz, Methanol-d₄) δ 8.69 (d, J=3.5 Hz, 1H), 8.27-8.18 (m, 1H), 8.14-8.02 (m, 2H), 7.93-7.88 (m, 1H), 7.86 (d, J=2.9 Hz, 1H), 7.84-7.81 (m, 2H), 7.59 (q, J=5.1, 3.9 Hz, 2H), 7.42-7.37 (m, 1H), 7.38-7.31 (m, 1H), 7.29-7.15 (m, 3H), 7.03 (dt, J=8.9, 2.2 Hz, 2H), 3.81-3.63 (m, 2H), 3.38 (d, J=4.7 Hz, 2H), 3.21-3.17 (m, 2H), 2.57-2.53 (m, 2H), 2.52-2.40 (m, 2H), 2.28 (t, J=2.7 Hz, 3H), 1.73-1.68 (m, 2H), 1.63 (d, J=10.5 Hz, 2H), 1.50 (d, J=9.1 Hz, 2H), 1.37 (d, J=6.9 Hz, 3H), 1.31 (d, J=5.2 Hz, 10H), 1.28 (t, J=3.0 Hz, 1H), 0.73 (q, J=6.9, 6.2 Hz, 3H). MS (ESI) m/z 995.6 [M+H]⁺.

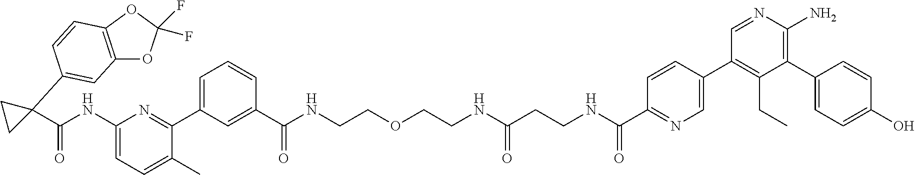 [SMILES: CCc1c(-c2ccc(C(=O)NCCC(=O)NCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 176: 6′-amino-N-(3-((2-(2-(3-(6-(1-(2,2-difluorobenzo[d][1,3]dioxol-5-yl)cyclopropane-1-carboxamido)-3-methylpyridin-2-yl)benzamido)ethoxy)ethyl)amino)-3-oxopropyl)-4′-ethyl-5′-(4-hydroxyphenyl)-[3,3′-bipyridine]-6-carboxamide (KL180-133)

Example 176 was synthesized following similar procedure for preparing example 167 from linker 93 and intermediate 7. White solid, 4.0 mg, 64% yield. ¹H NMR (400 MHz, Methanol-d₄) δ 8.65 (d, J=3.3 Hz, 1H), 8.18 (dd, J=6.4, 3.9 Hz, 1H), 8.11-7.99 (m, 2H), 7.95-7.85 (m, 2H), 7.85-7.75 (m, 2H), 7.55 (d, J=4.0 Hz, 2H), 7.39 (d, J=3.2 Hz, 1H), 7.34 (dd, J=8.8, 2.9 Hz, 1H), 7.26-7.17 (m, 3H), 7.03 (dt, J=8.6, 2.5 Hz, 2H), 3.64 (dd, J=9.7, 5.1 Hz, 4H), 3.55 (s, 4H), 3.41 (t, J=4.8 Hz, 2H), 2.50-2.38 (m, 4H), 2.26 (t, J=2.5 Hz, 3H), 1.72-1.68 (m, 2H), 1.32 (t, J=2.6 Hz, 2H), 1.29-1.25 (m, 2H), 0.76-0.67 (m, 3H). MS (ESI) m/z 927.4 [M+H]⁻.

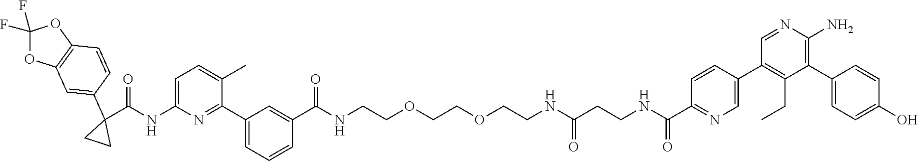 [SMILES: CCc1c(-c2ccc(C(=O)NCCC(=O)NCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 177: 6′-amino-N-(1-(3-(6-(1-(2,2-difluorobenzo[d][1,3]dioxol-5-yl)cyclopropane-1-carboxamido)-3-methylpyridin-2-yl)phenyl)-1,12-dioxo-5,8-dioxa-2,11-diazatetradecan-14-yl)-4′-ethyl-5′-(4-hydroxyphenyl)-[3,3′-bipyridine]-6-carboxamide (KL180-134)

Example 177 was synthesized following similar procedure for preparing example 167 from linker 94 and intermediate 7. White solid, 5.7 mg, 59% yield. ¹H NMR (400 MHz, Methanol-d₄) δ 8.66 (d, J=3.1 Hz, 1H), 8.25-8.16 (m, 1H), 8.10-8.03 (m, 2H), 7.95-7.86 (m, 2H), 7.82 (t, J=4.5 Hz, 2H), 7.58 (d, J=4.1 Hz, 2H), 7.40 (d, J=3.1 Hz, 1H), 7.34 (dd, J=8.9, 2.8 Hz, 1H), 7.22 (ddt, J=13.6, 8.5, 2.4 Hz, 3H), 7.03 (dt, J=8.6, 2.4 Hz, 2H), 3.72-3.65 (m, 4H), 3.62 (dd, J=11.6, 5.4 Hz, 4H), 3.54 (d, J=4.8 Hz, 2H), 2.52 (q, J=5.6, 4.5 Hz, 2H), 2.44 (d, J=8.4 Hz, 2H), 2.31-2.24 (m, 3H), 2.18 (t, J=2.4 Hz, 1H), 1.72-1.68 (m, 2H), 1.32 (t, J=2.5 Hz, 3H), 1.29-1.26 (m, 2H), 0.78-0.70 (m, 3H). MS (ESI) m/z 971.4 [M+H]⁺.

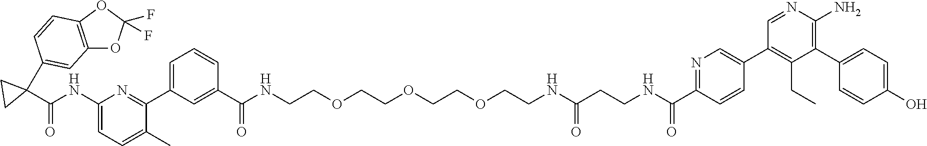 [SMILES: CCc1c(-c2ccc(C(=O)NCCC(=O)NCCOCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 178: 6′-amino-N-(1-(3-(6-(1-(2,2-difluorobenzo[d][1,3]dioxol-5-yl)cyclopropane-1-carboxamido)-3-methylpyridin-2-yl)phenyl)-1,15-dioxo-5,8,11-trioxa-2,14-diazaheptadecan-17-yl)-4′-ethyl-5′-(4-hydroxyphenyl)-[3,3′-bipyridine]-6-carboxamide (KL180-135)

Example 178 was synthesized following similar procedure for preparing example 167 from linker 95 and intermediate 7. White solid, 6.4 mg, 63% yield. ¹H NMR (400 MHz, Methanol-d₄) δ 8.66 (s, 1H), 8.24-8.17 (m, 1H), 8.13-7.99 (m, 2H), 7.95-7.90 (m, 1H), 7.88 (s, 1H), 7.87-7.80 (m, 2H), 7.62-7.52 (m, 2H), 7.40 (t, J=2.2 Hz, 1H), 7.35 (dd, J=8.9, 3.2 Hz, 1H), 7.27-7.14 (m, 3H), 7.03 (dt, J=8.5, 2.6 Hz, 2H), 3.73-3.64 (m, 8H), 3.61 (d, J=4.8 Hz, 4H), 3.54 (t, J=4.2 Hz, 2H), 3.48 (t, J=5.0 Hz, 2H), 2.58-2.49 (m, 2H), 2.49-2.38 (m, 2H), 2.28 (t, J=2.6 Hz, 3H), 1.72-1.69 (m, 2H), 1.33-1.32 (m, 2H), 1.29-1.26 (m, 2H), 0.74-0.70 (m, 3H). MS (ESI) m/z 1015.4 [M+H]⁺.

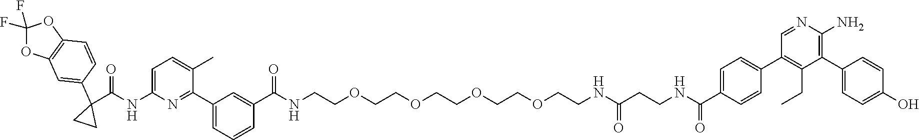 [SMILES: CCc1c(-c2ccc(C(=O)NCCC(=O)NCCOCCOCCOCCOCCNC(=O)c3cccc(-c4nc(NC(=O)C5(c6ccc7c(c6)OC(F)(F)O7)CC5)ccc4C)c3)cc2)cnc(N)c1-c1ccc(O)cc1]

### Example 179: 6′-amino-N-(1-(3-(6-(1-(2,2-difluorobenzo[d][1,3]dioxol-5-yl)cyclopropane-1-carboxamido)-3-methylpyridin-2-yl)phenyl)-1,18-dioxo-5,8,11,14-tetraoxa-2,17-diazaicosan-20-yl)-4′-ethyl-5′-(4-hydroxyphenyl)-[3,3′-bipyridine]-6-carboxamide (KL180-136)

Example 179 was synthesized following similar procedure for preparing example 167 from linker 96 and intermediate 7. White solid, 6.1 mg, 58% yield. ¹H NMR (400 MHz, Methanol-d₄) δ 8.68 (d, J=3.3 Hz, 1H), 8.24-8.16 (m, 1H), 8.11-8.01 (m, 2H), 7.96-7.91 (m, 1H), 7.89 (d, J=2.9 Hz, 1H), 7.87-7.80 (m, 2H), 7.64-7.53 (m, 2H), 7.43-7.38 (m, 1H), 7.39-7.31 (m, 1H), 7.29-7.12 (m, 3H), 7.03 (dt, J=8.5, 2.6 Hz, 2H), 3.74-3.49 (m, 19H), 3.37 (d, J=4.3 Hz, 1H), 2.57 (dt, J=9.0, 5.2 Hz, 2H), 2.49-2.40 (m, 2H), 2.28 (t, J=2.6 Hz, 3H), 1.73-1.69 (m, 2H), 1.32 (t, J=2.6 Hz, 2H), 1.29 (q, J=2.9 Hz, 2H), 0.73 (q, J=7.0, 6.5 Hz, 3H). MS (ESI) m/z [M+H]⁺.

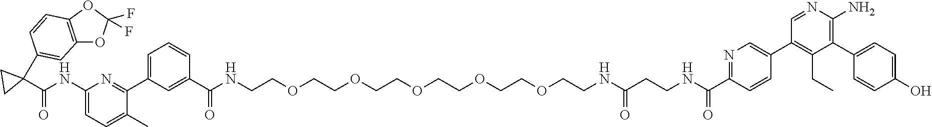 [SMILES: CCc1c(-c2ccc(C(=O)NCCC(=O)NCCOCCOCCOCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 180: 6′-amino-N-(1-(3-(6-(1-(2,2-difluorobenzo[d][1,3]dioxol-5-yl)cyclopropane-1-carboxamido)-3-methylpyridin-2-yl)phenyl)-1,21-dioxo-5,8,11,14,17-pentaoxa-2,20-diazatricosan-23-yl)-4′-ethyl-5′-(4-hydroxyphenyl)-[3,3′-bipyridine]-6-carboxamide (KL180-137)

Example 180 was synthesized following similar procedure for preparing example 167 from linker 97 and intermediate 7. White solid, 5.4 mg, 49% yield. ¹H NMR (400 MHz, Methanol-d₄) δ 8.68 (d, J=3.3 Hz, 1H), 8.24-8.16 (m, 1H), 8.08 (td, J=11.7, 10.3, 4.3 Hz, 2H), 7.96-7.90 (m, 1H), 7.89 (d, J=3.1 Hz, 1H), 7.86-7.81 (m, 2H), 7.60 (d, J=4.1 Hz, 2H), 7.40 (t, J=2.2 Hz, 1H), 7.35 (dd, J=8.9, 3.1 Hz, 1H), 7.27-7.22 (m, 1H), 7.20 (dt, J=8.6, 2.2 Hz, 2H), 7.03 (dt, J=8.4, 2.5 Hz, 2H), 3.75-3.69 (m, 2H), 3.65 (d, J=3.0 Hz, 6H), 3.62-3.57 (m, 12H), 3.53 (t, J=5.0 Hz, 2H), 3.39 (t, J=4.9 Hz, 2H), 2.58 (q, J=4.1, 3.1 Hz, 2H), 2.48-2.42 (m, 2H), 2.28 (t, J=2.6 Hz, 3H), 1.73-1.69 (m, 2H), 1.32 (t, J=2.7 Hz, 2H), 1.28 (q, J=2.9 Hz, 2H), 0.75-0.71 (m, 3H). MS (ESI) m/z 1059.5 [M+H]⁺.

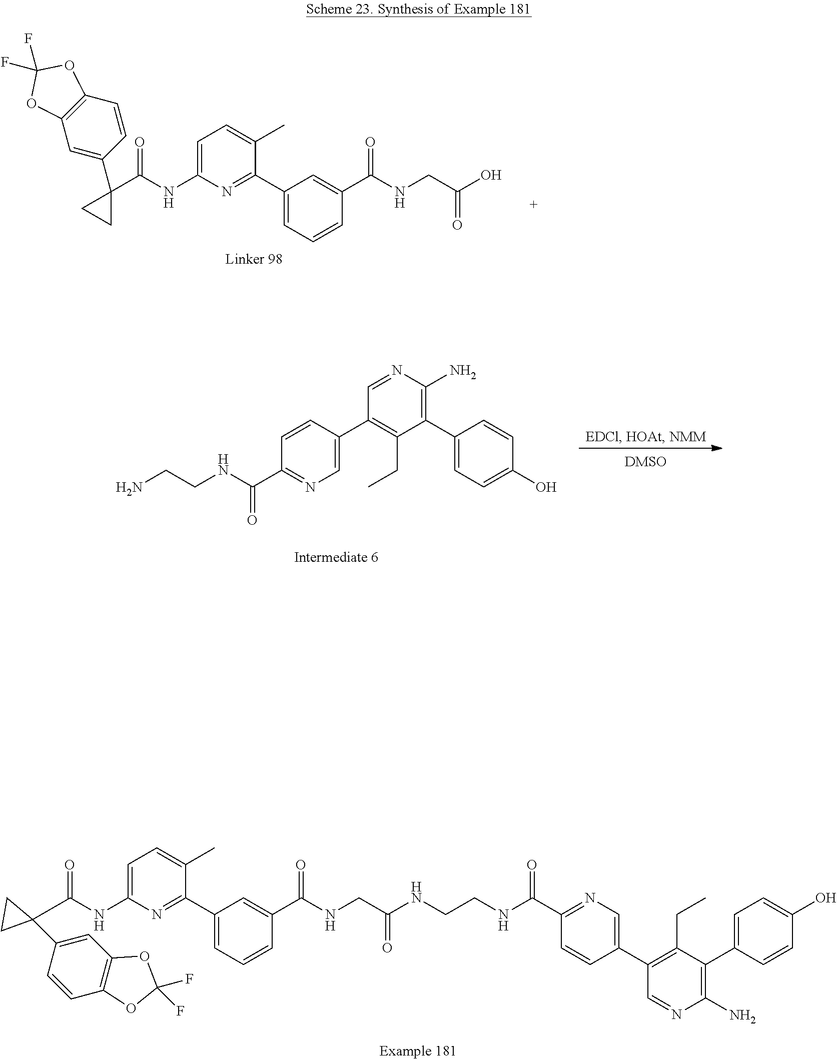 [SMILES: Cc1ccc(NC(=O)C2(c3ccc4c(c3)OC(F)(F)O4)CC2)nc1-c1cccc(C(=O)NCC(=O)O)c1 ; CCc1c(-c2ccc(C(=O)NCCN)nc2)cnc(N)c1-c1ccc(O)cc1 ; CCc1c(-c2ccc(C(=O)NCCNC(=O)CNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 181: 6′-amino-N-(2-(2-(3-(6-(1-(2,2-difluorobenzo[d][1,3]dioxol-5-yl)cyclopropane-1-carboxamido)-3-methylpyridin-2-yl)benzamido)acetamido)ethyl)-4′-ethyl-5′-(4-hydroxyphenyl)-[3,3′-bipyridine]-6-carboxamide (KL180-162)

To a solution of intermediate 6 (3.8 mg, 0.01 mmol) in DMSO (1 mL) were added linker 98 (5.1 mg, 0.01 mmol, 1.0 equiv), EDCI (1-ethyl-3-(3-dimethylaminopropyl)carbodiimide) (3.0 mg, 0.015 mmol, 1.5 equiv), HOAt (1-hydroxy-7-azabenzo-triazole) (2.0 mg, 0.015 mmol, 1.5 equiv), and NMM (N-Methylmorpholine) (3.0 mg, 0.03 mmol, 3.0 equiv). After being stirred overnight at room temperature, the resulting mixture was purified by preparative HPLC (5%-70% acetonitrile/0.1% TFA in H₂O) to afford titled compound as white solid (4.9 mg, 57%). ¹H NMR (400 MHz, Methanol-d₄) δ 8.65 (d, J=3.5 Hz, 1H), 8.12-8.07 (m, 2H), 7.97 (t, J=4.9 Hz, 3H), 7.80 (q, J=3.9, 2.4 Hz, 2H), 7.60 (d, J=10.3 Hz, 2H), 7.38 (t, J=2.3 Hz, 1H), 7.37-7.29 (m, 1H), 7.26-7.16 (m, 3H), 7.04 (dt, J=8.7, 2.6 Hz, 2H), 4.04 (t, J=2.6 Hz, 2H), 3.62 (q, J=5.4, 5.0 Hz, 2H), 3.51 (q, J=5.4 Hz, 2H), 2.49-2.35 (m, 2H), 2.28 (t, J=2.6 Hz, 3H), 1.71-1.67 (m, 2H), 1.29-1.25 (m, 2H), 0.70 (q, J=6.8, 5.3 Hz, 3H). MS (ESI) m/z 869.3 [M+H]⁺.

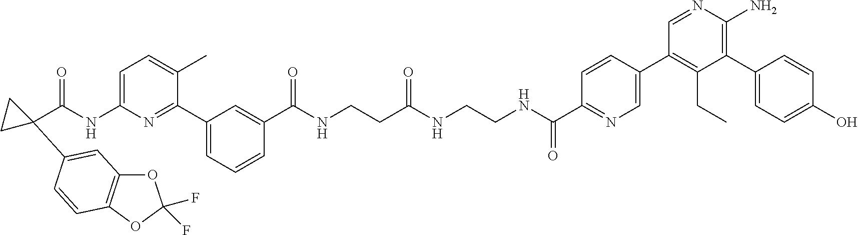 [SMILES: CCc1c(-c2ccc(C(=O)NCCNC(=O)CCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 182: 6′-amino-N-(2-(3-(3-(6-(1-(2,2-difluorobenzo[d][1,3]dioxol-5-yl)cyclopropane-1-carboxamido)-3-methylpyridin-2-yl)benzamido)propanamido)ethyl)-4′-ethyl-5′-(4-hydroxyphenyl)-[3,3′-bipyridine]-6-carboxamide (KL180-163)

Example 182 was synthesized following similar procedure for preparing example 181 from intermediate 6 and linker 99. White solid, 4.7 mg, 53% yield. ¹H NMR (400 MHz, Methanol-d₄) δ 8.65 (d, J=2.9 Hz, 1H), 8.16 (dt, J=8.0, 2.4 Hz, 1H), 8.12-8.05 (m, 1H), 8.01 (dt, J=8.4, 2.6 Hz, 1H), 7.91-7.83 (m, 2H), 7.79 (dt, J=11.6, 2.5 Hz, 2H), 7.64-7.47 (m, 2H), 7.42-7.36 (m, 1H), 7.34 (dd, J=9.4, 2.6 Hz, 1H), 7.28-7.14 (m, 3H), 7.03 (dt, J=8.6, 2.3 Hz, 2H), 3.65 (q, J=8.0, 6.7 Hz, 2H), 3.58 (q, J=5.1 Hz, 2H), 3.47 (d, J=4.9 Hz, 2H), 2.54 (q, J=6.2, 4.9 Hz, 2H), 2.49-2.38 (m, 2H), 2.26 (t, J=2.4 Hz, 3H), 1.71-1.67 (m, 2H), 1.29-1.25 (m, 2H), 0.83-0.63 (m, 3H). MS (ESI) m/z 883.3 [M+H]⁺.

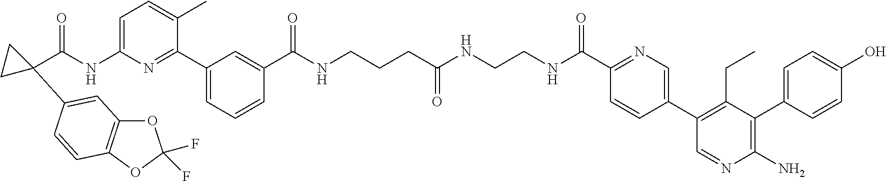 [SMILES: CCc1c(-c2ccc(C(=O)NCCNC(=O)CCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 183: 6′-amino-N-(2-(4-(3-(6-(1-(2,2-difluorobenzo[d][1,3]dioxol-5-yl)cyclopropane-1-carboxamido)-3-methylpyridin-2-yl)benzamido)butanamido)ethyl)-4′-ethyl-5′-(4-hydroxyphenyl)-[3,3′-bipyridine]-6-carboxamide (KL180-164)

Example 183 was synthesized following similar procedure for preparing example 181 from intermediate 6 and linker 100. White solid, 4.9 mg, 55% yield. ¹H NMR (400 MHz, Methanol-d₄) δ 8.63 (s, 1H), 8.24-8.14 (m, 1H), 8.07 (dt, J=8.9, 2.6 Hz, 1H), 8.04-7.97 (m, 1H), 7.89 (dd, J=11.3, 4.8 Hz, 2H), 7.83-7.75 (m, 2H), 7.57 (d, J=9.5 Hz, 2H), 7.39 (d, J=3.3 Hz, 1H), 7.33 (dd, J=8.6, 3.0 Hz, 1H), 7.22 (dt, J=8.2, 2.4 Hz, 1H), 7.18 (dt, J=8.5, 2.3 Hz, 2H), 7.02 (dt, J=8.5, 2.2 Hz, 2H), 3.57 (t, J=5.0 Hz, 2H), 3.47 (t, J=5.1 Hz, 2H), 3.40 (d, J=16.7 Hz, 2H), 2.44-2.37 (m, 2H), 2.35-2.28 (m, 2H), 2.27 (t, J=2.5 Hz, 3H), 1.93-1.89 (m, 2H), 1.72-1.68 (m, 2H), 1.29-1.25 (m, 2H), 0.69 (q, J=7.9, 6.6 Hz, 3H). MS (ESI) m/z 897.5 [M+H]⁺.

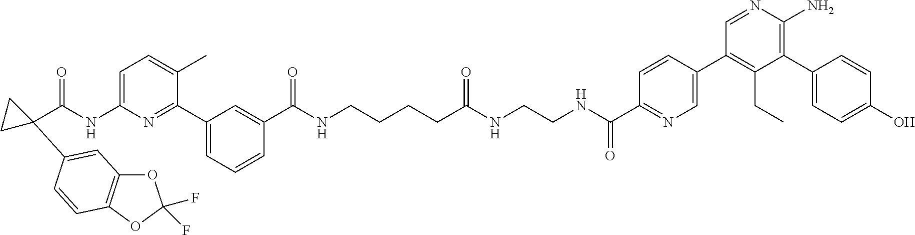 [SMILES: CCc1c(-c2ccc(C(=O)NCCNC(=O)CCCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 184: 6′-amino-N-(2-(5-(3-(6-(1-(2,2-difluorobenzo[d][1,3]dioxol-5-yl)cyclopropane-1-carboxamido)-3-methylpyridin-2-yl)benzamido)pentanamido)ethyl)-4′-ethyl-5′-(4-hydroxyphenyl)-[3,3′-bipyridine]-6-carboxamide (KL180-165)

Example 184 was synthesized following similar procedure for preparing example 181 from intermediate 6 and linker 101. White solid, 5.4 mg, 59% yield. ¹H NMR (400 MHz, Methanol-d₄) δ 8.66 (s, 1H), 8.23-8.14 (m, 1H), 8.13-8.05 (m, 1H), 8.02 (t, J=5.2 Hz, 1H), 7.92 (q, J=4.4, 3.8 Hz, 3H), 7.82 (d, J=2.9 Hz, 1H), 7.61 (d, J=10.3 Hz, 2H), 7.43-7.38 (m, 1H), 7.37-7.31 (m, 1H), 7.24 (t, J=5.2 Hz, 1H), 7.18 (t, J=5.4 Hz, 2H), 7.03 (dt, J=8.0, 2.7 Hz, 2H), 3.58 (t, J=5.2 Hz, 2H), 3.49 (t, J=5.1 Hz, 2H), 2.48-2.36 (m, 2H), 2.30 (d, J=3.1 Hz, 3H), 2.28 (d, J=5.4 Hz, 2H), 1.83-1.54 (m, 6H), 1.41 (q, J=5.6 Hz, 1H), 1.30 (q, J=3.0 Hz, 2H), 0.71 (d, J=4.7 Hz, 2H). MS (ESI) m/z 911.5

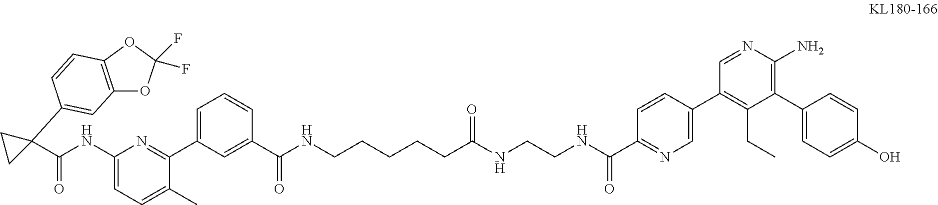 [SMILES: CCc1c(-c2ccc(C(=O)NCCNC(=O)CCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 185: 6′-amino-N-(2-(6-(3-(6-(1-(2,2-difluorobenzo[d][1,3]dioxol-5-yl)cyclopropane-1-carboxamido)-3-methylpyridin-2-yl)benzamido)hexanamido)ethyl)-4′-ethyl-5′-(4-hydroxyphenyl)-[3,3′-bipyridine]-6-carboxamide (KL180-166)

Example 185 was synthesized following similar procedure for preparing example 181 from intermediate 6 and linker 102. White solid, 5.4 mg, 58% yield. ¹H NMR (400 MHz, Methanol-d₄) δ 8.69 (d, J=3.1 Hz, 1H), 8.23-8.16 (m, 1H), 8.06 (ddt, J=18.8, 8.0, 2.4 Hz, 2H), 7.91-7.84 (m, 2H), 7.80 (dd, J=10.6, 3.0 Hz, 2H), 7.57 (d, J=4.3 Hz, 2H), 7.43-7.37 (m, 1H), 7.34 (dd, J=8.7, 3.0 Hz, 1H), 7.26-7.21 (m, 1H), 7.18 (dt, J=8.9, 2.2 Hz, 2H), 7.06-6.97 (m, 2H), 3.55 (t, J=5.1 Hz, 2H), 3.44 (q, J=5.5 Hz, 2H), 3.37 (s, 2H), 2.46-2.40 (m, 2H), 2.26 (t, J=2.5 Hz, 3H), 2.23 (d, J=9.2 Hz, 2H), 1.70 (q, J=3.0 Hz, 2H), 1.62 (dd, J=19.0, 9.0 Hz, 4H), 1.40 (q, J=7.6, 6.2 Hz, 2H), 1.30-1.25 (m, 2H), 0.71 (q, J=7.0, 5.9 Hz, 3H). MS (ESI) m/z 925.6 [M+H]⁺.

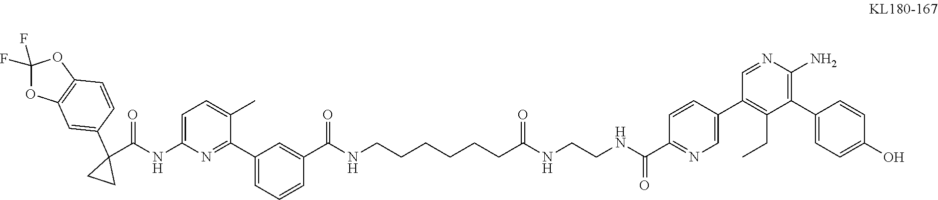 [SMILES: CCc1c(-c2ccc(C(=O)NCCNC(=O)CCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 186: 6′-amino-N-(2-(7-(3-(6-(1-(2,2-difluorobenzo[d][1,3]dioxol-5-yl)cyclopropane-1-carboxamido)-3-methylpyridin-2-yl)benzamido)heptanamido)ethyl)-4′-ethyl-5′-(4-hydroxyphenyl)-[3,3′-bipyridine]-6-carboxamide (KL180-167)

Example 186 was synthesized following similar procedure for preparing example 181 from intermediate 6 and linker 103. White solid, 4.8 mg, 52% yield. ¹H NMR (400 MHz, Methanol-d₄) δ 8.70 (d, J=3.3 Hz, 1H), 8.25-8.17 (m, 1H), 8.11-8.06 (m, 1H), 8.06-7.99 (m, 1H), 7.91-7.84 (m, 2H), 7.83-7.77 (m, 2H), 7.57 (q, J=5.5, 4.1 Hz, 2H), 7.43-7.37 (m, 1H), 7.37-7.30 (m, 1H), 7.24 (t, J=5.3 Hz, 1H), 7.18 (dd, J=7.3, 4.3 Hz, 2H), 7.02 (dt, J=8.7, 2.5 Hz, 2H), 3.58 (t, J=5.2 Hz, 2H), 3.47 (q, J=5.5 Hz, 2H), 3.37 (s, 2H), 2.47-2.41 (m, 2H), 2.27 (t, J=2.8 Hz, 3H), 2.25-2.19 (m, 2H), 1.70 (q, J=2.9 Hz, 2H), 1.60 (t, J=9.5 Hz, 4H), 1.43-1.31 (m, 4H), 1.30-1.25 (m, 2H), 0.78-0.67 (m, 3H). MS (ESI) m/z 939.6 [M+H]⁺.

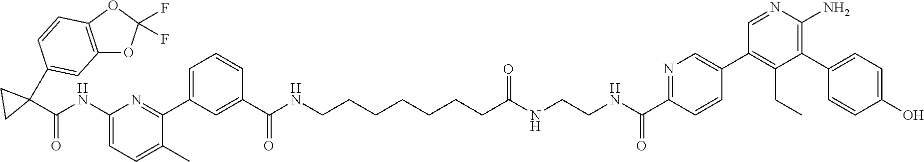 [SMILES: CCc1c(-c2ccc(C(=O)NCCNC(=O)CCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 187: 6′-amino-N-(2-(8-(3-(6-(1-(2,2-difluorobenzo[d][1,3]dioxol-5-yl)cyclopropane-1-carboxamido)-3-methylpyridin-2-yl)benzamido)octanamido)ethyl)-4′-ethyl-5′-(4-hydroxyphenyl)-[3,3′-bipyridine]-6-carboxamide (KL180-168)

Example 187 was synthesized following similar procedure for preparing example 181 from intermediate 6 and linker 104. White solid, 5.3 mg, 56% yield. ¹H NMR (400 MHz, Methanol-d₄) δ 8.70 (d, J=3.4 Hz, 1H), 8.24-8.15 (m, 1H), 8.12-8.03 (m, 2H), 7.92-7.83 (m, 2H), 7.84-7.74 (m, 2H), 7.62-7.50 (m, 2H), 7.44-7.38 (m, 1H), 7.38-7.31 (m, 1H), 7.28-7.21 (m, 1H), 7.21-7.15 (m, 2H), 7.08-6.98 (m, 2H), 3.57 (t, J=5.2 Hz, 2H), 3.47 (t, J=5.1 Hz, 2H), 3.37 (d, J=6.2 Hz, 2H), 2.48-2.42 (m, 2H), 2.27 (t, J=2.8 Hz, 3H), 2.21 (q, J=7.1, 6.4 Hz, 2H), 1.70 (q, J=3.0 Hz, 2H), 1.63-1.57 (m, 4H), 1.35 (s, 6H), 1.28 (q, J=3.0 Hz, 2H), 0.75-0.71 (m, 3H). MS (ESI) m/z 953.6 [M+H]⁺.

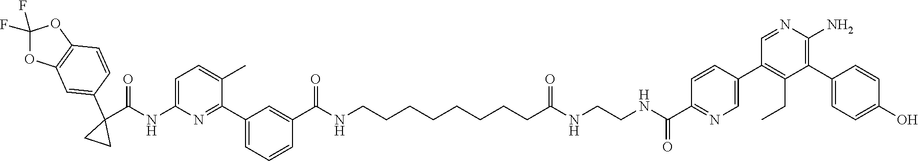 [SMILES: CCc1c(-c2ccc(C(=O)NCCNC(=O)CCCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 188: 6′-amino-N-(2-(9-(3-(6-(1-(2,2-difluorobenzo[d][1,3]dioxol-5-yl)cyclopropane-1-carboxamido)-3-methylpyridin-2-yl)benzamido)nonanamido)ethyl)-4′-ethyl-5′-(4-hydroxyphenyl)-[3,3′-bipyridine]-6-carboxamide (KL180-169)

Example 188 was synthesized following similar procedure for preparing example 181 from intermediate 6 and linker 105. White solid, 6.2 mg, 64% yield. ¹H NMR (400 MHz, Methanol-d₄) δ 8.70 (d, J=3.5 Hz, 1H), 8.25-8.17 (m, 1H), 8.13-8.01 (m, 2H), 7.92-7.84 (m, 2H), 7.81 (dd, J=6.8, 4.6 Hz, 2H), 7.58 (q, J=6.2, 4.3 Hz, 2H), 7.40 (t, J=2.3 Hz, 1H), 7.37-7.32 (m, 1H), 7.24 (dd, J=7.1, 4.2 Hz, 1H), 7.19 (dt, J=8.7, 2.2 Hz, 2H), 7.05-6.97 (m, 2H), 3.58 (q, J=5.5 Hz, 2H), 3.46 (q, J=5.5 Hz, 2H), 3.41-3.35 (m, 2H), 2.52-2.35 (m, 2H), 2.28 (t, J=2.8 Hz, 3H), 2.20 (q, J=6.8, 5.1 Hz, 2H), 1.72-1.68 (m, 2H), 1.61 (d, J=9.3 Hz, 4H), 1.33 (d, J=9.8 Hz, 8H), 1.28 (q, J=3.0 Hz, 2H), 0.75-0.71 (m, 3H). MS (ESI) m/z 967.6 [M+H]⁺.

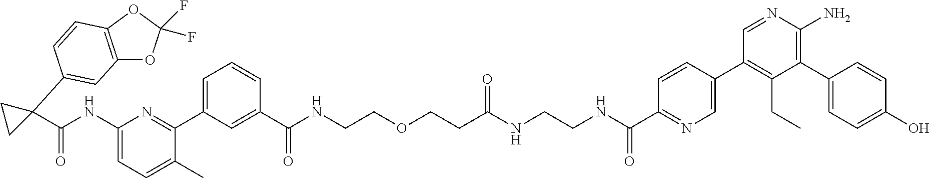 [SMILES: CCc1c(-c2ccc(C(=O)NCCNC(=O)CCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 189: 6′-amino-N-(2-(3-(2-(3-(6-(1-(2,2-difluorobenzo[d][1,3]dioxol-5-yl)cyclopropane-1-carboxamido)-3-methylpyridin-2-yl)benzamido)ethoxy)propanamido)-ethyl)-4′-ethyl-5′-(4-hydroxyphenyl)-[3,3′-bipyridine]-6-carboxamide (KL180-170)

Example 189 was synthesized following similar procedure for preparing example 181 from intermediate 6 and linker 106. White solid, 5.3 mg, 57% yield. ¹H NMR (400 MHz, Methanol-d₄) δ 8.62 (d, J=3.2 Hz, 1H), 8.17 (dt, J=8.2, 2.3 Hz, 1H), 8.10-8.04 (m, 1H), 8.05-7.98 (m, 1H), 7.90 (t, J=5.9 Hz, 2H), 7.85-7.75 (m, 2H), 7.59-7.47 (m, 2H), 7.41-7.35 (m, 1H), 7.35-7.29 (m, 1H), 7.26-7.14 (m, 3H), 7.03 (dt, J=8.8, 2.2 Hz, 2H), 3.74 (q, J=5.2 Hz, 2H), 3.62 (t, J=4.7 Hz, 2H), 3.55 (q, J=4.9 Hz, 2H), 3.44 (q, J=5.4 Hz, 2H), 2.48-2.40 (m, 4H), 2.26 (t, J=2.5 Hz, 3H), 1.71-1.67 (m, 2H), 1.29-1.25 (m, 2H), 0.71 (q, J=7.2, 6.7 Hz, 3H). MS (ESI) m/z 927.5 [M+H]⁺.

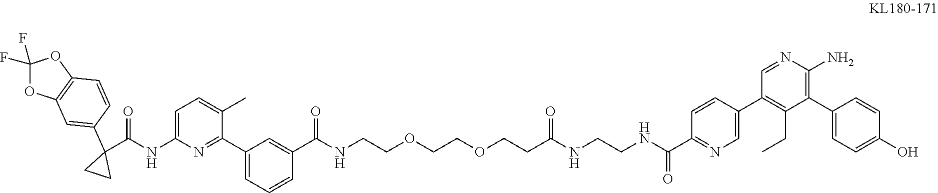 [SMILES: CCc1c(-c2ccc(C(=O)NCCNC(=O)CCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 190: 6′-amino-N-(1-(3-(6-(1-(2,2-difluorobenzo[d][1,3]dioxol-5-yl)cyclopropane-1-carboxamido)-3-methylpyridin-2-yl)phenyl)-1,11-dioxo-5,8-dioxa-2,12-diazatetradecan-14-yl)-4′-ethyl-5′-(4-hydroxyphenyl)-[3,3′-bipyridine]-6-carboxamide (KL180-171)

Example 190 was synthesized following similar procedure for preparing example 181 from intermediate 6 and linker 107. White solid, 5.0 mg, 51% yield. ¹H NMR (400 MHz, Methanol-d₄) δ 8.67 (d, J=3.6 Hz, 1H), 8.24-8.14 (m, 1H), 8.08 (dt, J=8.5, 2.7 Hz, 1H), 8.06-8.00 (m, 1H), 7.95-7.85 (m, 2H), 7.85-7.72 (m, 2H), 7.57 (q, J=4.6, 3.5 Hz, 2H), 7.43-7.38 (m, 1H), 7.37-7.31 (m, 1H), 7.27-7.14 (m, 3H), 7.03 (dt, J=8.7, 2.6 Hz, 2H), 3.72 (q, J=5.5 Hz, 2H), 3.64 (t, J=4.9 Hz, 2H), 3.63-3.49 (m, 8H), 3.43 (t, J=5.3 Hz, 2H), 2.47-2.40 (m, 4H), 2.32-2.20 (m, 3H), 1.72-1.68 (m, 2H), 1.27 (q, J=3.0 Hz, 2H), 0.74-0.70 (m, 3H). MS (ESI) m/z 971.6 [M+H]⁺.

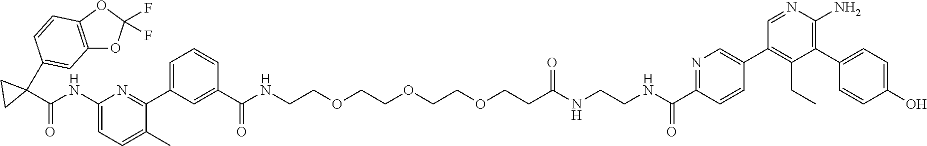 [SMILES: CCc1c(-c2ccc(C(=O)NCCNC(=O)CCOCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 191: 6′-amino-N-(1-(3-(6-(1-(2,2-difluorobenzo[d][1,3]dioxol-5-yl)cyclopropane-1-carboxamido)-3-methylpyridin-2-yl)phenyl)-1,14-dioxo-5,8,11-trioxa-2,15-diazaheptadecan-17-yl)-4′-ethyl-5′-(4-hydroxyphenyl)-[3,3′-bipyridine]-6-carboxamide (KL180-172)

Example 191 was synthesized following similar procedure for preparing example 181 from intermediate 6 and linker 108. White solid, 5.5 mg, 54% yield. ¹H NMR (400 MHz, Methanol-d₄) δ 8.68 (d, J=3.5 Hz, 1H), 8.20 (q, J=3.1 Hz, 1H), 8.07 (ddd, J=16.2, 9.3, 3.1 Hz, 2H), 7.96-7.86 (m, 2H), 7.83 (dd, J=11.0, 2.6 Hz, 2H), 7.62-7.55 (m, 2H), 7.43-7.38 (m, 1H), 7.34 (dd, J=8.7, 3.1 Hz, 1H), 7.25-7.18 (m, 3H), 7.03 (dt, J=8.7, 2.4 Hz, 2H), 3.73-3.61 (m, 8H), 3.60-3.54 (m, 8H), 3.45 (t, J=5.2 Hz, 2H), 2.47-2.40 (m, 4H), 2.32-2.25 (m, 3H), 1.72-1.69 (m, 2H), 1.28 (q, J=2.9 Hz, 2H), 0.74-0.70 (m, 3H). MS (ESI) m/z 1015.6 [M+H]⁺.

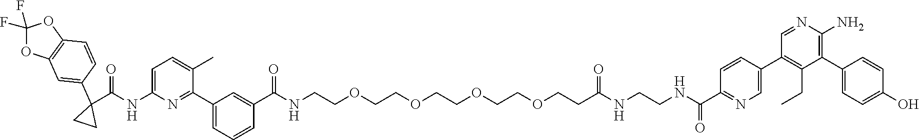 [SMILES: CCc1c(-c2ccc(C(=O)NCCNC(=O)CCOCCOCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 192: 6′-amino-N-(1-(3-(6-(1-(2,2-difluorobenzo[d][1,3]dioxol-5-yl)cyclopropane-1-carboxamido)-3-methylpyridin-2-yl)phenyl)-1,17-dioxo-5,8,11,14-tetraoxa-2,18-diazaicosan-20-yl)-4′-ethyl-5′-(4-hydroxyphenyl)-[3,3′-bipyridine]-6-carboxamide (KL180-173)

Example 192 was synthesized following similar procedure for preparing example 181 from intermediate 6 and linker 109. White solid, 6.1 mg, 58% yield. ¹H NMR (400 MHz, Methanol-d₄) δ 8.69 (s, 1H), 8.25-8.16 (m, 1H), 8.13-8.01 (m, 2H), 7.94-7.85 (m, 2H), 7.85-7.78 (m, 2H), 7.63-7.52 (m, 2H), 7.44-7.37 (m, 1H), 7.37-7.32 (m, 1H), 7.28-7.17 (m, 3H), 7.03 (dt, J=8.9, 2.3 Hz, 2H), 3.74-3.62 (m, 8H), 3.63-3.50 (m, 12H), 3.46 (q, J=5.6 Hz, 2H), 2.45 (q, J=4.3 Hz, 4H), 2.28 (t, J=2.6 Hz, 3H), 1.71-1.68 (m, 2H), 1.30-1.26 (m, 2H), 0.79-0.66 (m, 3H). MS (ESI) m/z 1059.6 [M+H]⁺.

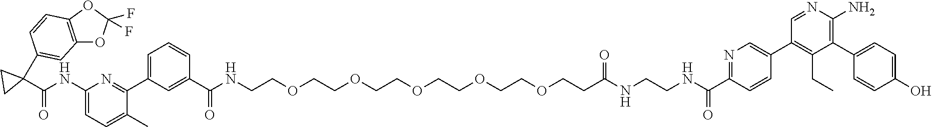 [SMILES: CCc1c(-c2ccc(C(=O)NCCNC(=O)CCOCCOCCOCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1]

### Example 193: 6′-amino-N-(1-(3-(6-(1-(2,2-difluorobenzo[d][1,3]dioxol-5-yl)cyclopropane-1-carboxamido)-3-methylpyridin-2-yl)phenyl)-1,20-dioxo-5,8,11,14,17-pentaoxa-2,21-diazatricosan-23-yl)-4′-ethyl-5′-(4-hydroxyphenyl)-[3,3′-bipyridine]-6-carboxamide (KL180-174)

Example 193 was synthesized following similar procedure for preparing example 181 from intermediate 6 and linker 110. White solid, 5.8 mg, 53% yield. ¹H NMR (400 MHz, Methanol-d₄) δ 8.70 (s, 1H), 8.22 (d, J=7.5 Hz, 1H), 8.13-8.01 (m, 2H), 7.98-7.87 (m, 2H), 7.83 (q, J=5.1, 3.9 Hz, 2H), 7.59 (q, J=6.2, 4.3 Hz, 2H), 7.40 (t, J=2.4 Hz, 1H), 7.38-7.31 (m, 1H), 7.29-7.13 (m, 3H), 7.03 (dt, J=8.6, 2.6 Hz, 2H), 3.71 (q, J=5.9 Hz, 2H), 3.65 (t, J=2.8 Hz, 6H), 3.63-3.55 (m, 14H), 3.47 (q, J=5.6 Hz, 2H), 2.48-2.42 (m, 4H), 2.28 (t, J=2.7 Hz, 3H), 1.70 (q, J=3.0 Hz, 2H), 1.31-1.27 (m, 2H), 0.75-0.71 (m, 3H). MS (ESI) m/z 1103.7 [M+H]⁺.

Example AMPK based DUBTACs are set forth in Table 1 below.

TABLE 1

| Example | | | |
|---|---|---|---|
| No. | Cpd ID | Structure | name |
| 1 | XH181- 1 | 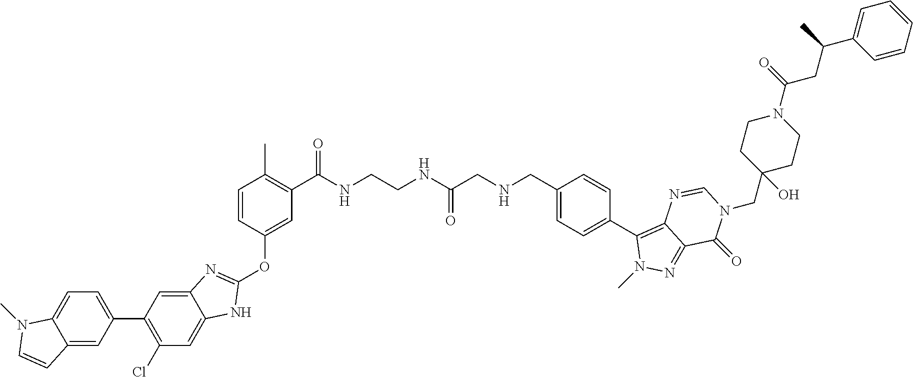 [SMILES: Cc1ccc(Oc2nc3cc(-c4ccc5c(ccn5C)c4)c(Cl)cc3[nH]2)cc1C(=O)NCCNC(=O)CNCc1ccc(-c2c3ncn(CC4(O)CCN(C(=O)C[C@@H](C)c5ccccc5)CC4)c(=O)c3nn2C)cc1] | (R)-5-((6- chloro-5-(1- methyl-1H- indol-5-yl)-1H- benzo[d]imidazol- 2-yl)oxy)-N- (2-(2-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) acetamido)eth- yl)-2- methylbenzamide |
| 2 | XH181- 2 | 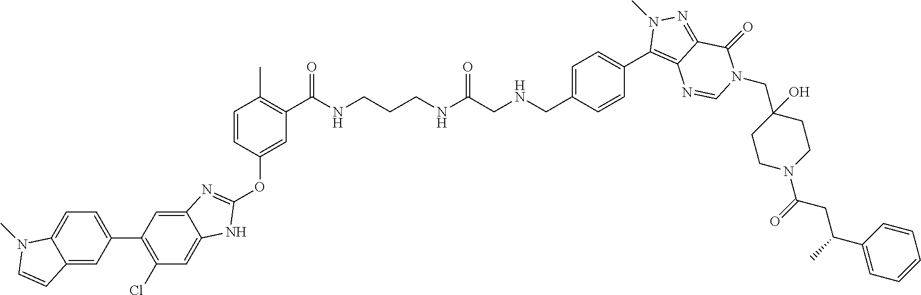 [SMILES: Cc1ccc(Oc2nc3cc(-c4ccc5c(ccn5C)c4)c(Cl)cc3[nH]2)cc1C(=O)NCCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N-(3- (2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)propyl)- 2- methylbenzamide |
| 3 | XH181- 3 | 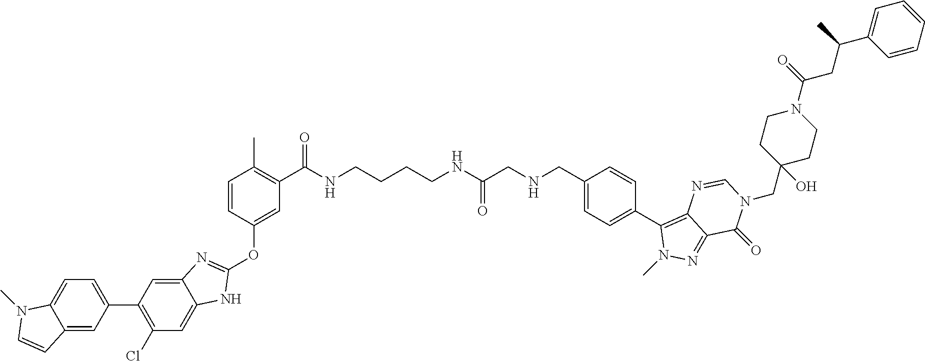 [SMILES: Cc1ccc(Oc2nc3cc(-c4ccc5c(ccn5C)c4)c(Cl)cc3[nH]2)cc1C(=O)NCCCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N-(4- (2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)butyl)- 2- methylbenzamide |
| 4 | XH181- 4 | 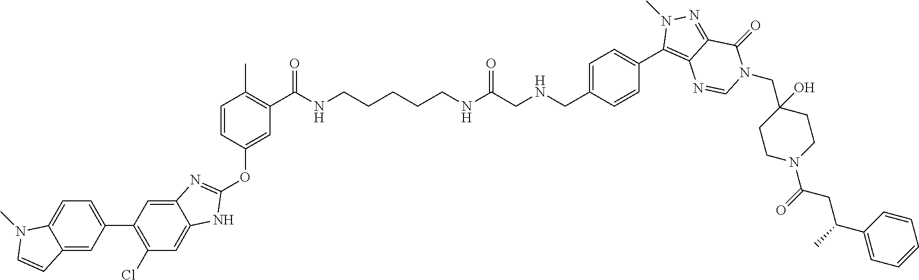 [SMILES: Cc1ccc(Oc2nc3cc(-c4ccc5c(ccn5C)c4)c(Cl)cc3[nH]2)cc1C(=O)NCCCCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N-(5- (2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)pentyl)- 2- |
| | | | methylbenzamide |
| 5 | XH181- 5 | 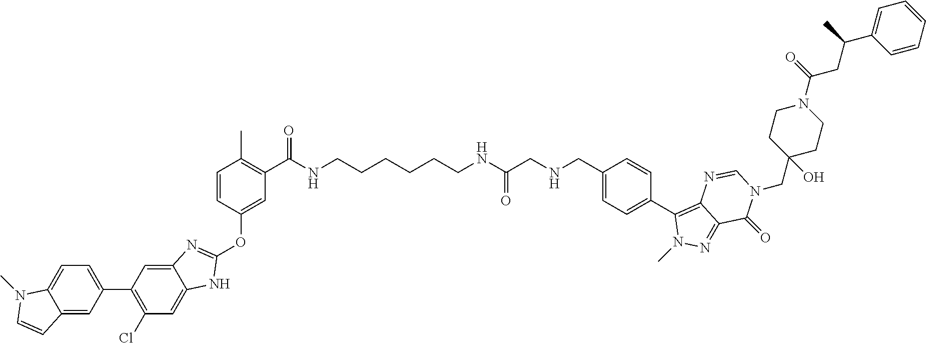 [SMILES: Cc1ccc(Oc2nc3cc(-c4ccc5c(ccn5C)c4)c(Cl)cc3[nH]2)cc1C(=O)NCCCCCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N-(6- (2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)hexyl)- 2- methylbenzamide |
| 6 | XH181- 6 | 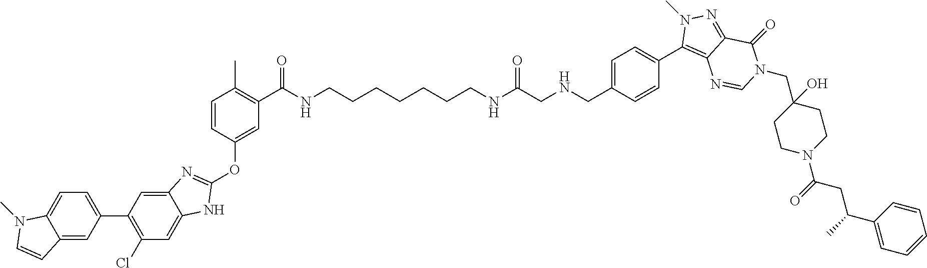 [SMILES: Cc1ccc(Oc2nc3cc(-c4ccc5c(ccn5C)c4)c(Cl)cc3[nH]2)cc1C(=O)NCCCCCCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N-(7- (2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)heptyl)- 2- |
| | | | methylbenzamide |
| 7 | XH181- 7 | 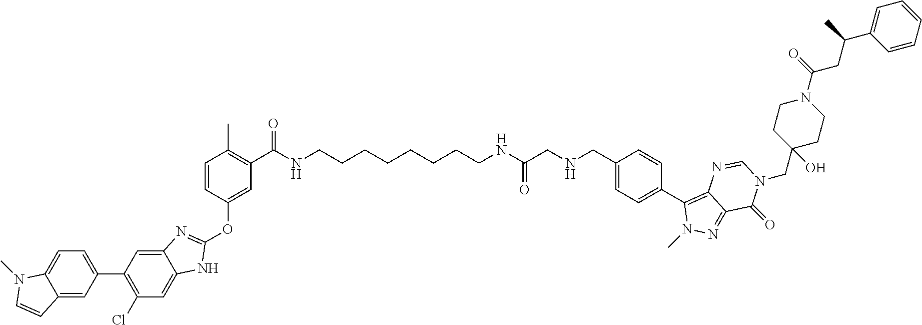 [SMILES: Cc1ccc(Oc2nc3cc(-c4ccc5c(ccn5C)c4)c(Cl)cc3[nH]2)cc1C(=O)NCCCCCCCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N-(8- (2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)octyl)- 2- methylbenzamide |
| 8 | XH181- 8 | 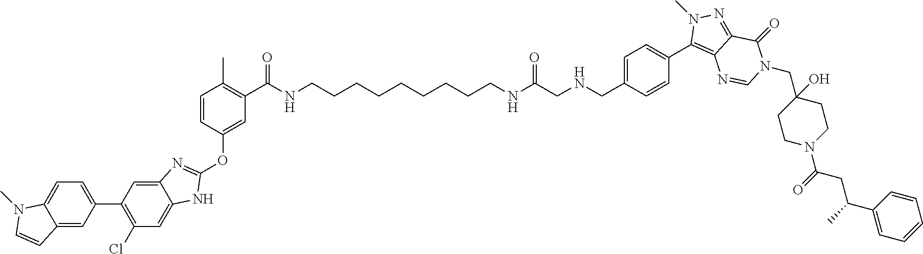 [SMILES: Cc1ccc(Oc2nc3cc(-c4ccc5c(ccn5C)c4)c(Cl)cc3[nH]2)cc1C(=O)NCCCCCCCCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N-(9- (2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)nonyl)- |
| | | | 2- |
| | | | methylbenzamide |
| 9 | XH181- 9 | 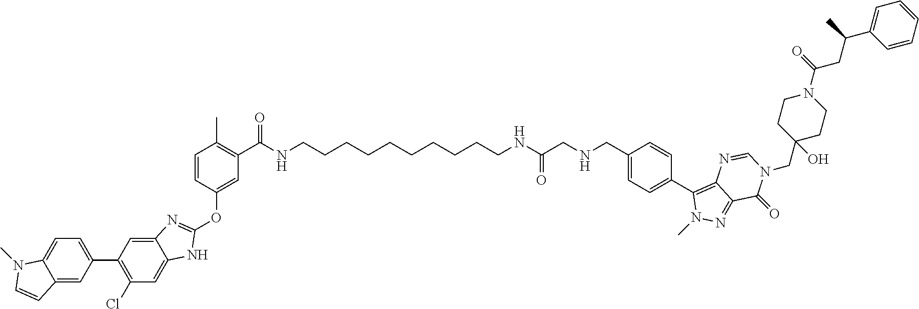 [SMILES: Cc1ccc(Oc2nc3cc(-c4ccc5c(ccn5C)c4)c(Cl)cc3[nH]2)cc1C(=O)NCCCCCCCCCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N- (10-(2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)decyl)- 2- methylbenzamide |
| 10 | XH181- 15 | 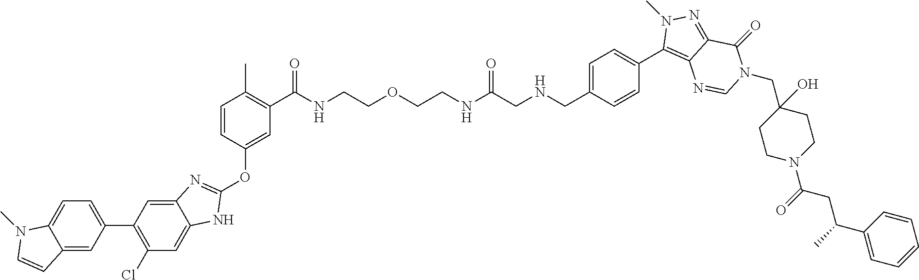 [SMILES: Cc1ccc(Oc2nc3cc(-c4ccc5c(ccn5C)c4)c(Cl)cc3[nH]2)cc1C(=O)NCCOCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N-(2- (2-(2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)ethoxy) ethyl)-2- |
| | | | methylbenzamide |
| 11 | XH181- 16 | 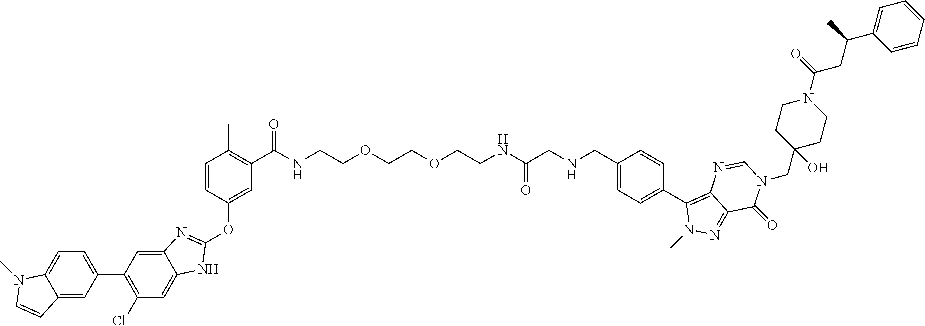 [SMILES: Cc1ccc(Oc2nc3cc(-c4ccc5c(ccn5C)c4)c(Cl)cc3[nH]2)cc1C(=O)NCCOCCOCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N-(1- (4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)phenyl)-4- oxo-8,11-dioxa- 2,5- diazatridecan- 13-yl)-2- methylbenzamide |
| 12 | XH181- 17 | 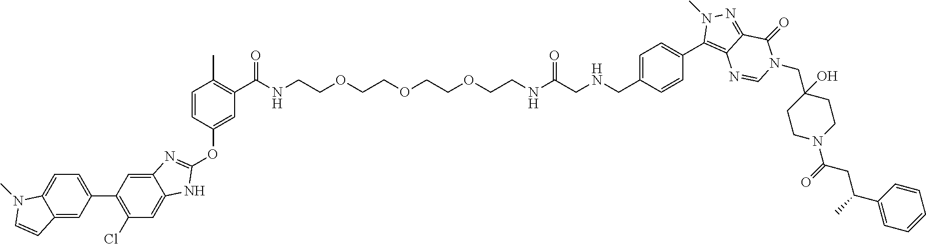 [SMILES: Cc1ccc(Oc2nc3cc(-c4ccc5c(ccn5C)c4)c(Cl)cc3[nH]2)cc1C(=O)NCCOCCOCCOCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N-(1- (4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)phenyl)-4- |
| | | | oxo-8,11,14- |
| | | | trioxa-2,5- |
| | | | diazahexadecan- |
| | | | 16-yl)-2- |
| | | | methylbenzamide |
| 13 | XH181- 18 (MS8118) | 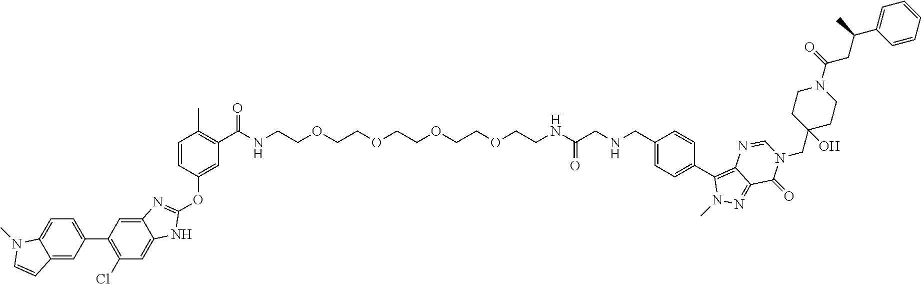 [SMILES: Cc1ccc(Oc2nc3cc(-c4ccc5c(ccn5C)c4)c(Cl)cc3[nH]2)cc1C(=O)NCCOCCOCCOCCOCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N-(1- (4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)phenyl)-4- oxo-8,11,14,17- tetraoxa-2,5- diazanonadecan- |
| | | | 19-yl)-2- |
| | | | methylbenzamide |
| 14 | XH181- 19 | 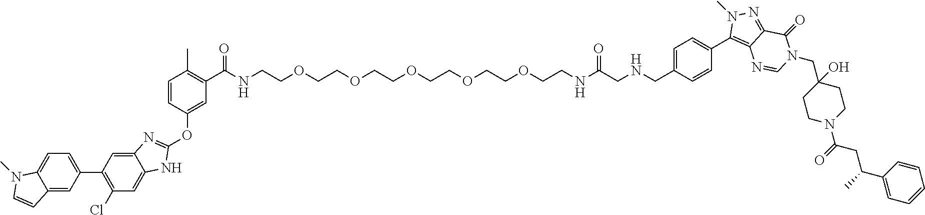 [SMILES: Cc1ccc(Oc2nc3cc(-c4ccc5c(ccn5C)c4)c(Cl)cc3[nH]2)cc1C(=O)NCCOCCOCCOCCOCCOCCNC(=O)CNCc1ccc(-c2c3ncn(CC4(O)CCN(C(=O)C[C@@H](C)c5ccccc5)CC4)c(=O)c3nn2C)cc1] | (R)-5-((6-chloro- 5-(1-methyl-1H- indol-5-yl)-1H- benzo[d]imidazol- 2-yl)oxy)-N-(1- (4-(6-((4- hydroxy-1-(3- phenylbutanoyl) piperidin-4- yl)methyl)-2- methyl-7-oxo- 6,7-dihydro-2H- pyrazolo[4,3- |
| | | | d]pyrimidin-3- |
| | | | yl)phenyl)-4- |
| | | | oxo- |
| | | | 8,11,14,17,20- |
| | | | pentaoxa-2,5- |
| | | | diazadocosan- |
| | | | 22-yl)-2- |
| | | | methylbenzamide |
| 15 | KL186- 1 | 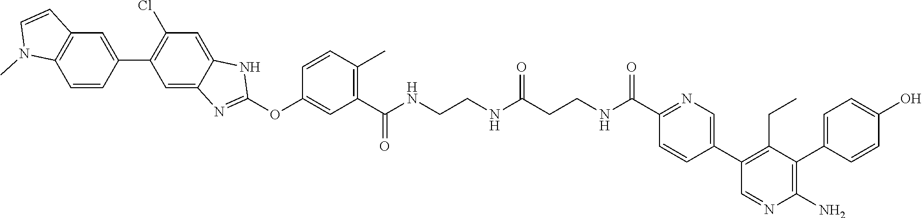 [SMILES: CCc1c(-c2ccc(C(=O)NCCC(=O)NCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(3- ((2-(5-((6-chloro- 5-(1-methyl-1H- indol-5-yl)-1H- benzo[d]imidazol- 2-yl)oxy)-2- methylbenzamido) ethyl)amino)- 3-oxopropyl)-4′- ethyl-5′-(4- hydroxyphenyl)- [3,3′- bipyridine]-6- carboxamide |
| 16 | KL186- 2 | 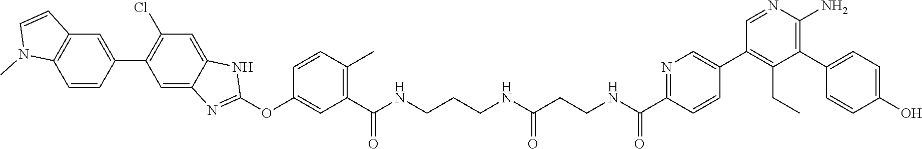 [SMILES: CCc1c(-c2ccc(C(=O)NCCC(=O)NCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(3- ((3-(5-((6-chloro- 5-(1-methyl-1H- indol-5-yl)-1H- benzo[d]imidazol- 2-yl)oxy)-2- methylbenzamido) propyl)amino)- 3-oxopropyl)- |
| | | | 4′-ethyl-5′-(4- |
| | | | hydroxyphenyl)- |
| | | | [3,3′- |
| | | | bipyridine]-6- |
| | | | carboxamide |
| 17 | KL186- 3 | 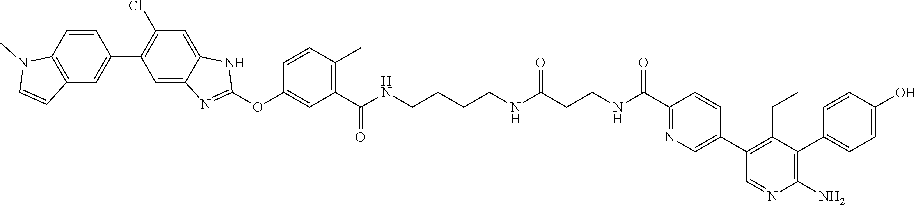 [SMILES: CCc1c(-c2ccc(C(=O)NCCC(=O)NCCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(3- ((4-(5-((6-chloro- 5-(1-methyl-1H- indol-5-yl)-1H- benzo[d]imidazol- 2-yl)oxy)-2- methylbenzamido) butyl)amino)- 3-oxopropyl)-4′- ethyl-5′-(4- hydroxyphenyl)- [3,3′- bipyridine]-6- |
| | | | carboxamide |
| 18 | KL186- 4 | 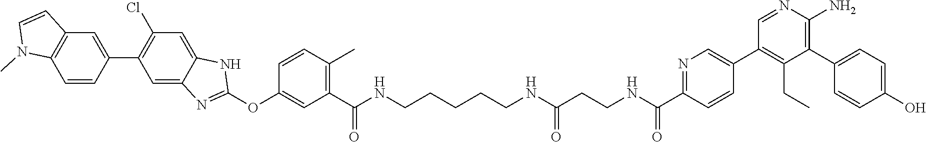 [SMILES: CCc1c(-c2ccc(C(=O)NCCC(=O)NCCCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(3- ((5-(5-((6-chloro- 5-(1-methyl-1H- indol-5-yl)-1H- benzo[d]imidazol- 2-yl)oxy)-2- methylbenzamido) pentyl)amino)- 3-oxopropyl)-4′- |
| | | | ethyl-5′-(4- |
| | | | hydroxyphenyl)- |
| | | | [3,3′- |
| | | | bipyridine]-6- |
| | | | carboxamide |
| 19 | KL186- 5 | 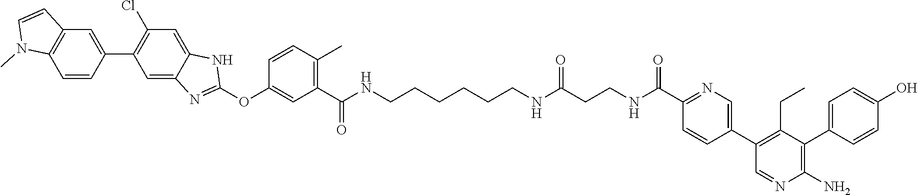 [SMILES: CCc1c(-c2ccc(C(=O)NCCC(=O)NCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(3- ((6-(5-((6-chloro- 5-(1-methyl-1H- indol-5-yl)-1H- benzo[d]imidazol- 2-yl)oxy)-2- methylbenzamido) hexyl)amino)- 3-oxopropyl)-4′- ethyl-5′-(4- hydroxyphenyl)- [3,3′- |
| | | | bipyridine]-6- |
| | | | carboxamide |
| 20 | KL186- 6 | 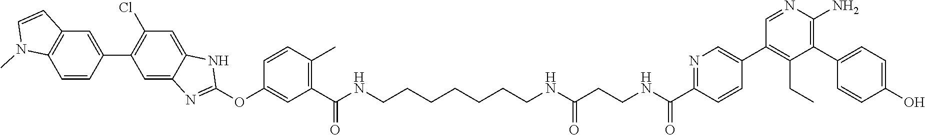 [SMILES: CCc1c(-c2ccc(C(=O)NCCC(=O)NCCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(3- ((7-(5-((6-chloro- 5-(1-methyl-1H- indol-5-yl)-1H- benzo[d]imidazol- 2-yl)oxy)-2- methylbenzamido) heptyl)amino)- 3-oxopropyl)-4′- |
| | | | ethyl-5′-(4- |
| | | | hydroxyphenyl)- |
| | | | [3,3′- |
| | | | bipyridine]-6- |
| | | | carboxamide |
| 21 | KL186- 7 | 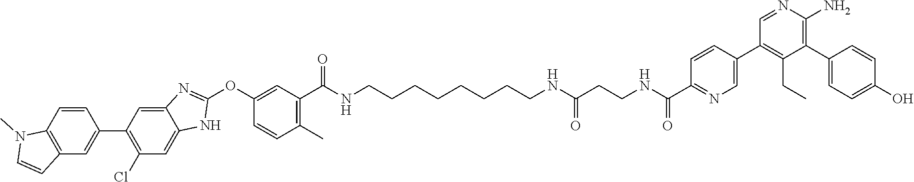 [SMILES: CCc1c(-c2ccc(C(=O)NCCC(=O)NCCCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(3- ((8-(5-((6-chloro- 5-(1-methyl-1H- indol-5-yl)-1H- benzo[d]imidazol- 2-yl)oxy)-2- methylbenzamido) octyl)amino)- 3-oxopropyl)-4′- ethyl-5′-(4- hydroxyphenyl)- [3,3′- |
| | | | bipyridine]-6- |
| | | | carboxamide |
| 22 | KL186- 8 | 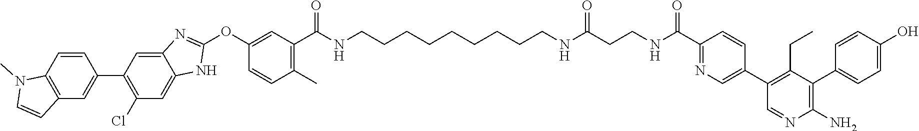 [SMILES: CCc1c(-c2ccc(C(=O)NCCC(=O)NCCCCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(3- ((9-(5-((6-chloro- 5-(1-methyl-1H- indol-5-yl)-1H- benzo[d]imidazol- 2-yl)oxy)-2- methylbenzamido) nonyl)amino)- 3-oxopropyl)-4′- |
| | | | ethyl-5′-(4- |
| | | | hydroxyphenyl)- |
| | | | [3,3′- |
| | | | bipyridine]-6- |
| | | | carboxamide |
| 23 | KL186- 9 | 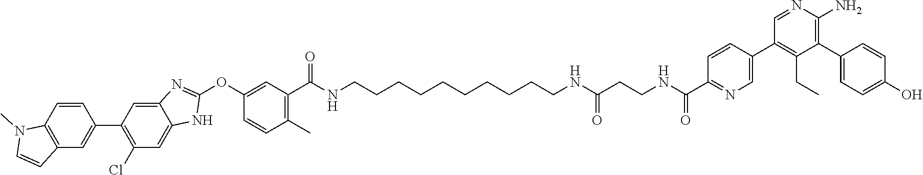 [SMILES: CCc1c(-c2ccc(C(=O)NCCC(=O)NCCCCCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(3- ((10-(5-((6- chloro-5-(1- methyl-1H- indol-5-yl)-1H- benzo[d]imidazol- 2-yl)oxy)-2- methylbenzamido) decyl)amino)- 3-oxopropyl)-4′- ethyl-5′-(4- |
| | | | hydroxyphenyl)- |
| | | | [3,3′- |
| | | | bipyridine]-6- |
| | | | carboxamide |
| 24 | KL186- 10 | 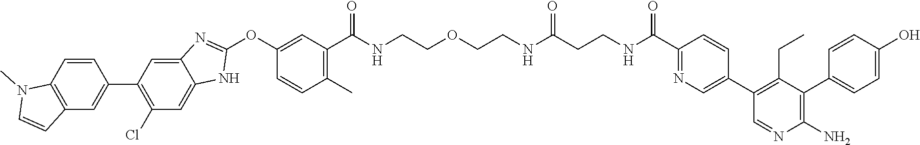 [SMILES: CCc1c(-c2ccc(C(=O)NCCC(=O)NCCOCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(3- ((2-(2-(5-((6- chloro-5-(1- methyl-1H- indol-5-yl)-1H- benzo[d]imidazol- 2-yl)oxy)-2- methylbenzamido) ethoxy)ethyl)a- |
| | | | mino)-3- |
| | | | oxopropyl)-4′- |
| | | | ethyl-5′-(4- |
| | | | hydroxyphenyl)- |
| | | | [3,3′- |
| | | | bipyridine]-6- |
| | | | carboxamide |
| 25 | KL186- 11 | 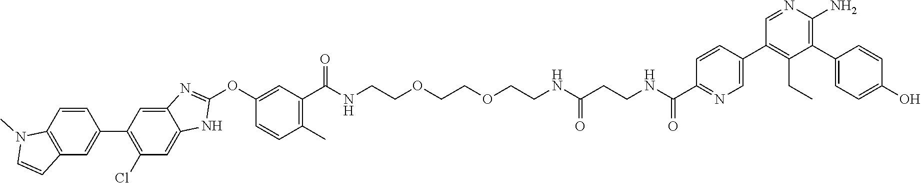 [SMILES: CCc1c(-c2ccc(C(=O)NCCC(=O)NCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(1- (5-((6-chloro-5- (1-methyl-1H- indol-5-yl)-1H- benzo[d]imidazol- 2-yl)oxy)-2- methylphenyl)- 1,12-dioxo-5,8- dioxa-2,11- diazatetradecan- 14-yl)-4′-ethyl- 5′-(4- |
| | | | hydroxyphenyl)- |
| | | | [3,3′- |
| | | | bipyridine]-6- |
| | | | carboxamide |
| 26 | KL186- 12 | 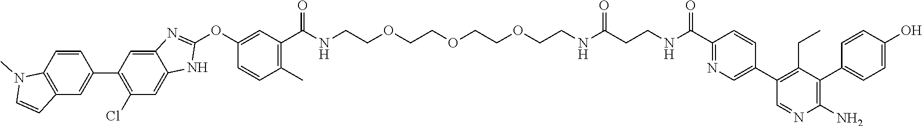 [SMILES: CCc1c(-c2ccc(C(=O)NCCC(=O)NCCOCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(1- (5-((6-chloro-5- (1-methyl-1H- indol-5-yl)-1H- benzo[d]imidazol- 2-yl)oxy)-2- methylphenyl)- 1,15-dioxo- |
| | | | 5,8,11-trioxa- |
| | | | 2,14- |
| | | | diazaheptadecan- |
| | | | 17-yl)-4′-ethyl- |
| | | | 5′-(4- |
| | | | hydroxyphenyl)- |
| | | | [3,3′- |
| | | | bipyridine]-6- |
| | | | carboxamide |
| 27 | KL186- 13 | 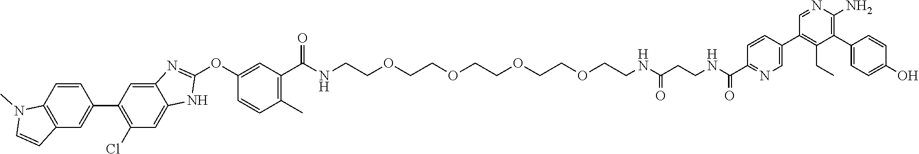 [SMILES: CCc1c(-c2ccc(C(=O)NCCC(=O)NCCOCCOCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(1- (5-((6-chloro-5- (1-methyl-1H- indol-5-yl)-1H- benzo[d]imidazol- 2-yl)oxy)-2- methylphenyl)- 1,18-dioxo- 5,8,11,14- tetraoxa-2,17- |
| | | | diazaicosan-20- |
| | | | yl)-4′-ethyl-5′- |
| | | | (4- |
| | | | hydroxyphenyl)- |
| | | | [3,3′- |
| | | | bipyridine]-6- |
| | | | carboxamide |
| 28 | KL186- 14 | 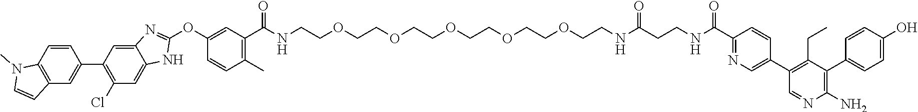 [SMILES: CCc1c(-c2ccc(C(=O)NCCC(=O)NCCOCCOCCOCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(1- (5-((6-chloro-5- (1-methyl-1H- indol-5-yl)-1H- benzo[d]imidazol- 2-yl)oxy)-2- methylphenyl)- |
| | | | 1,21-dioxo- |
| | | | 5,8,11,14,17- |
| | | | pentaoxa-2,20- |
| | | | diazatricosan- |
| | | | 23-yl)-4′-ethyl- |
| | | | 5′-(4- |
| | | | hydroxyphenyl)- |
| | | | [3,3′- |
| | | | bipyridine]-6- |
| | | | carboxamide |
| 29 | KL186- 48 | 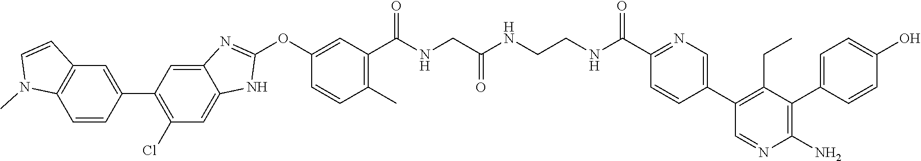 [SMILES: CCc1c(-c2ccc(C(=O)NCCNC(=O)CNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(2- (2-(5-((6-chloro- 5-(1-methyl-1H- indol-5-yl)-1H- benzo[d]imidazol- 2-yl)oxy)-2- methylbenzamido) acetamido)eth- yl)-4′-ethyl-5′- (4- |
| | | | hydroxyphenyl)- |
| | | | [3,3′- |
| | | | bipyridine]-6- |
| | | | carboxamide |
| 30 | KL186- 49 | 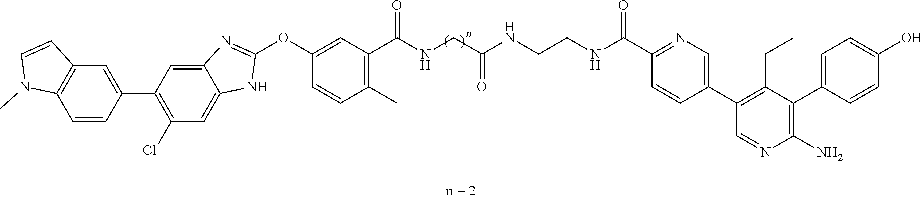 | 6′-amino-N-(2- (3-(5-((6-chloro- 5-(1-methyl-1H- indol-5-yl)-1H- benzo[d]imidazol- 2-yl)oxy)-2- methylbenzamido) propanamido) ethyl)-4′-ethyl- 5′-(4- hydroxyphenyl)- [3,3′- |
| | | | bipyridine]-6- |
| | | | carboxamide |
| 31 | KL186- 50 | 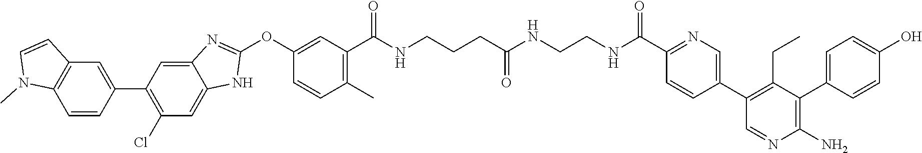 [SMILES: CCc1c(-c2ccc(C(=O)NCCNC(=O)CCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(2- (4-(5-((6-chloro- 5-(1-methyl-1H- indol-5-yl)-1H- benzo[d]imidazol- 2-yl)oxy)-2- methylbenzamido) butanamido)eth- yl)-4′-ethyl-5′- (4- |
| | | | hydroxyphenyl)- |
| | | | [3,3′- |
| | | | bipyridine]-6- |
| | | | carboxamide |
| 32 | KL186- 51 | 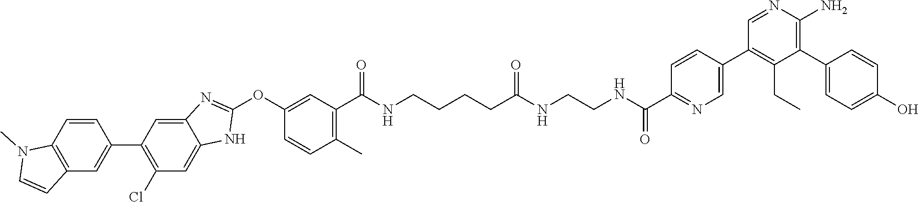 [SMILES: CCc1c(-c2ccc(C(=O)NCCNC(=O)CCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(2- (5-(5-((6-chloro- 5-(1-methyl-1H- indol-5-yl)-1H- benzo[d]imidazol- 2-yl)oxy)-2- methylbenzamido) pentanamido)eth- yl)-4′-ethyl-5′- (4- hydroxyphenyl)- [3,3′- bipyridine]-6- |
| | | | carboxamid |
| 33 | KL186- 52 | 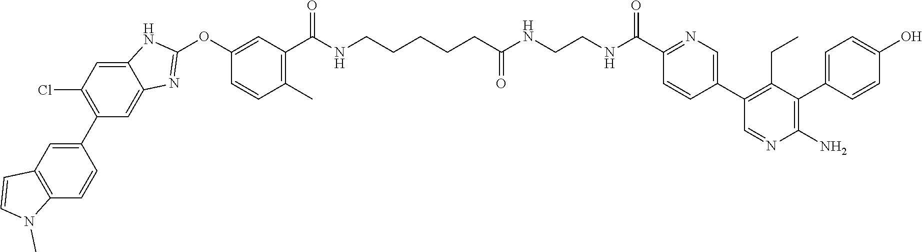 [SMILES: CCc1c(-c2ccc(C(=O)NCCNC(=O)CCCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(2- (6-(5-((6-chloro- 5-(1-methyl-1H- indol-5-yl)-1H- benzo[d]imidazol- 2-yl)oxy)-2- methylbenzamido) hexanamido)eth- yl)-4′-ethyl-5′- (4- hydroxyphenyl)- [3,3′- bipyridine]-6- carboxamide |
| 34 | KL186- 53 | 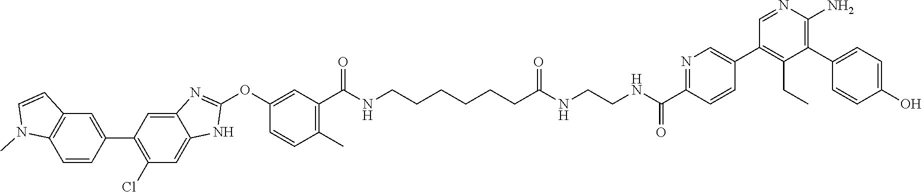 [SMILES: CCc1c(-c2ccc(C(=O)NCCNC(=O)CCCCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(2- (7-(5-((6-chloro- 5-(1-methyl-1H- indol-5-yl)-1H- benzo[d]imidazol- 2-yl)oxy)-2- methylbenzamido) heptanamido)eth- yl)-4′-ethyl-5′- (4- hydroxyphenyl)- [3,3′- |
| | | | bipyridine]-6- |
| | | | carboxamide |
| 35 | KL186- 54 | 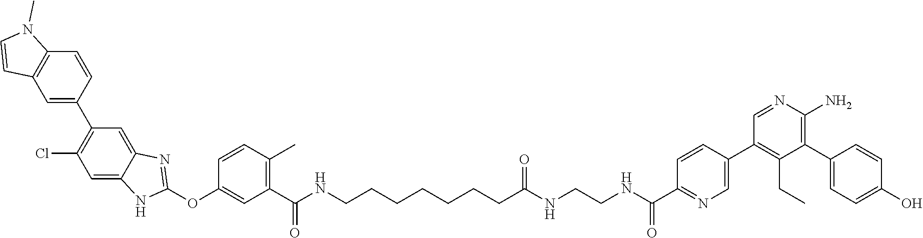 [SMILES: CCc1c(-c2ccc(C(=O)NCCNC(=O)CCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(2- (8-(5-((6-chloro- 5-(1-methyl-1H- indol-5-yl)-1H- benzo[d]imidazol- 2-yl)oxy)-2- methylbenzamido) octanamido)eth- yl)-4′-ethyl-5′- (4- hydroxyphenyl)- [3,3′- bipyridine]-6- carboxamide |
| 36 | KL186- 55 (MS8655) | 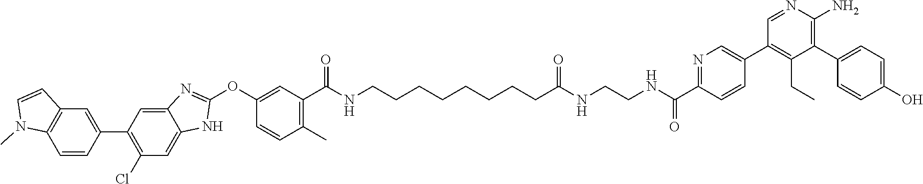 [SMILES: CCc1c(-c2ccc(C(=O)NCCNC(=O)CCCCCCCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(2- (9-(5-((6-chloro- 5-(1-methyl-1H- indol-5-yl)-1H- benzo[d]imidazol- 2-yl)oxy)-2- methylbenzamido) nonanamido)eth- yl)-4′-ethyl-5′- (4- hydroxyphenyl)- [3,3′- |
| | | | bipyridine]-6- |
| | | | carboxamide |
| 37 | KL186- 56 (MS8656) | 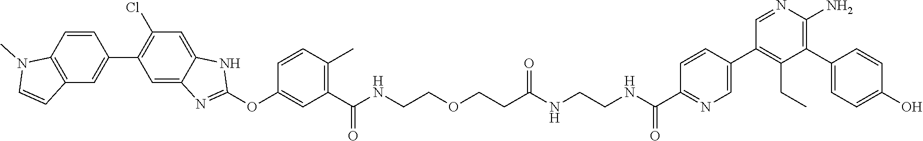 [SMILES: CCc1c(-c2ccc(C(=O)NCCNC(=O)CCOCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(2- (3-(2-(5-((6- chloro-5-(1- methyl-1H- indol-5-yl)-1H- benzo[d]imidazol- 2-yl)oxy)-2- methylbenzamido) ethoxy)propan- |
| | | | amido)ethyl)-4′- |
| | | | ethyl-5′-(4- |
| | | | hydroxyphenyl)- |
| | | | [3,3′- |
| | | | bipyridine]-6- |
| | | | carboxamide |
| 38 | KL186- 57 (MS8657) | 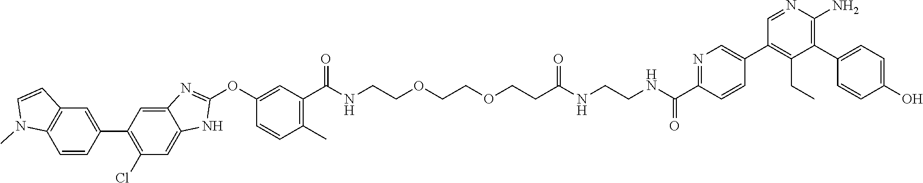 [SMILES: CCc1c(-c2ccc(C(=O)NCCNC(=O)CCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(1- (5-((6-chloro-5- (1-methyl-1H- indol-5-yl)-1H- benzo[d]imidazol- 2-yl)oxy)-2- methylphenyl)- 1,11-dioxo-5,8- dioxa-2,12- diazatetradecan- 14-yl)-4′-ethyl- 5′-(4- |
| | | | hydroxyphenyl)- |
| | | | [3,3′- |
| | | | bipyridine]-6- |
| | | | carboxamide |
| 39 | KL186- 58 | 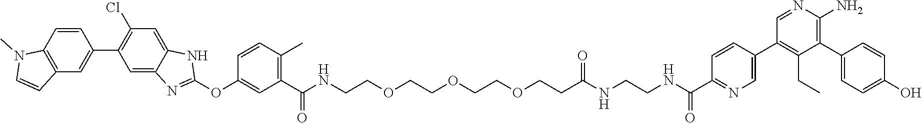 [SMILES: CCc1c(-c2ccc(C(=O)NCCNC(=O)CCOCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(1- (5-((6-chloro-5- (1-methyl-1H- indol-5-yl)-1H- benzo[d]imidazol- 2-yl)oxy)-2- methylphenyl)- 1,14-dioxo- |
| | | | 5,8,11-trioxa- |
| | | | 2,15- |
| | | | diazaheptadecan- |
| | | | 17-yl)-4′-ethyl- |
| | | | 5′-(4- |
| | | | hydroxyphenyl)- |
| | | | [3,3′- |
| | | | bipyridine]-6- |
| | | | carboxamide |
| 40 | KL186- 59 | 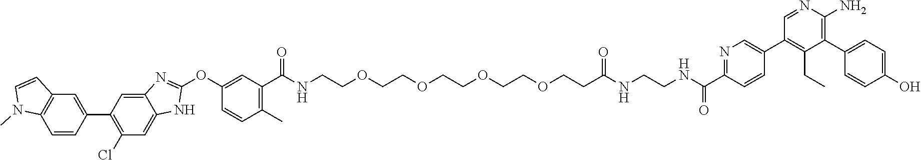 [SMILES: CCc1c(-c2ccc(C(=O)NCCNC(=O)CCOCCOCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(1- (5-((6-chloro-5- (1-methyl-1H- indol-5-yl)-1H- benzo[d]imidazol- 2-yl)oxy)-2- methylphenyl)- 1,17-dioxo- 5,8,11,14- tetraoxa-2,18- |
| | | | diazaicosan-20- |
| | | | yl)-4′-ethyl-5′- |
| | | | (4- |
| | | | hydroxyphenyl)- |
| | | | [3,3′- |
| | | | bipyridine]-6- |
| | | | carboxamide |
| 41 | KL186- 60 | 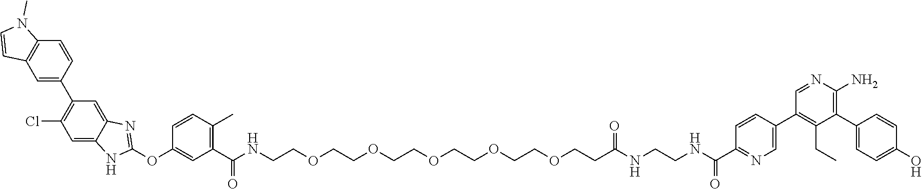 [SMILES: CCc1c(-c2ccc(C(=O)NCCNC(=O)CCOCCOCCOCCOCCOCCNC(=O)c3cc(Oc4nc5cc(-c6ccc7c(ccn7C)c6)c(Cl)cc5[nH]4)ccc3C)nc2)cnc(N)c1-c1ccc(O)cc1] | 6′-amino-N-(1- (5-((6-chloro-5- (1-methyl-1H- indol-5-yl)-1H- benzo[d]imidazol- 2-yl)oxy)-2- methylphenyl)- 1,20-dioxo- 5,8,11,14,17- pentaoxa-2,21- diazatricosan- 23-yl)-4′-ethyl- |
| | | | 5′-(4- |
| | | | hydroxyphenyl)- |
| | | | [3,3′- |
| | | | bipyridine]-6- |
| | | | carboxamide |

Example cGAS based DUBTACs are set forth in Table 2 below.

TABLE 2

| Example | | | |
|---|---|---|---|
| No. | Cpd ID | Structure | Name |
| 42 | XH168- 142 | 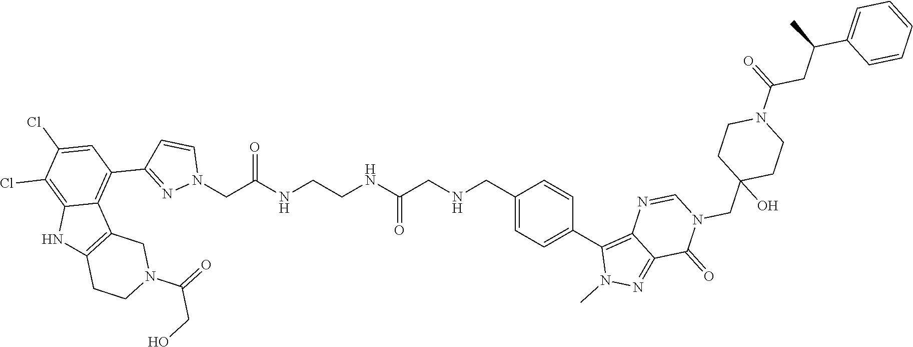 [SMILES: C[C@H](CC(=O)N1CCC(O)(Cn2cnc3c(-c4ccc(CNCC(=O)NCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (2-(2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)ethyl) acetamide |
| 43 | XH168- 143 | 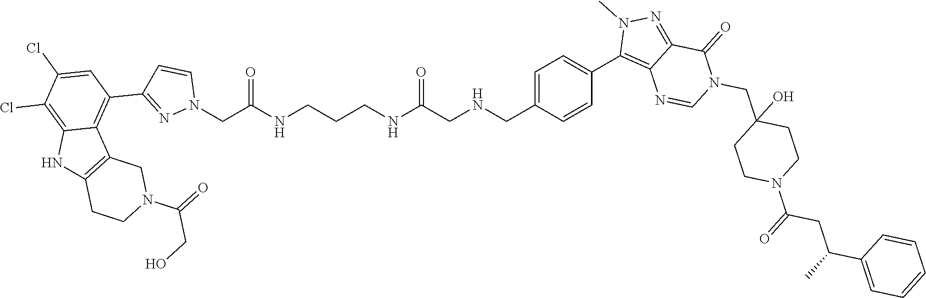 [SMILES: C[C@H](CC(=O)N1CCC(O)(Cn2cnc3c(-c4ccc(CNCC(=O)NCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (3-(2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)propyl) acetamide |
| 44 | XH168- 144 | 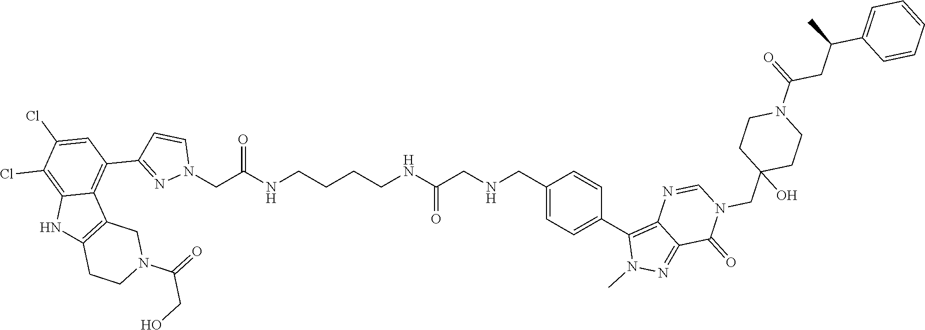 [SMILES: C[C@H](CC(=O)N1CCC(O)(Cn2cnc3c(-c4ccc(CNCC(=O)NCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (4-(2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)butyl) acetamide |
| 45 | XH168- 145 | 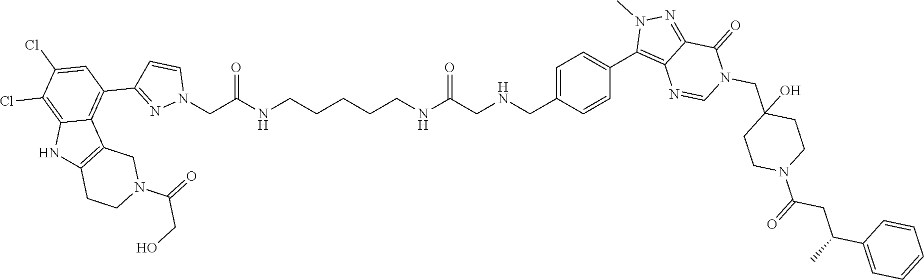 [SMILES: C[C@H](CC(=O)N1CCC(O)(Cn2cnc3c(-c4ccc(CNCC(=O)NCCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (5-(2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)pentyl) acetamide |
| 46 | XH168- 146 | 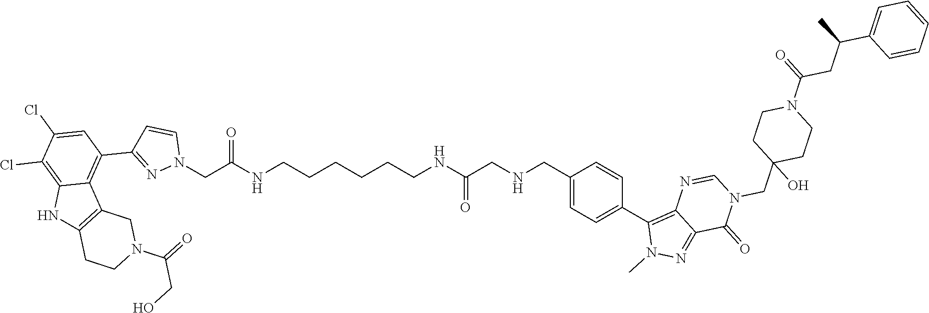 [SMILES: C[C@H](CC(=O)N1CCC(O)(Cn2cnc3c(-c4ccc(CNCC(=O)NCCCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (6-(2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)hexyl) acetamide |
| 47 | XH168- 147 | 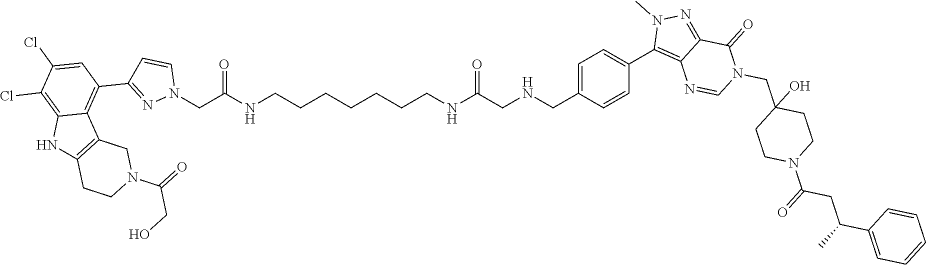 [SMILES: C[C@H](CC(=O)N1CCC(O)(Cn2cnc3c(-c4ccc(CNCC(=O)NCCCCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (7-(2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)heptyl) acetamide |
| 48 | XH168- 148 | 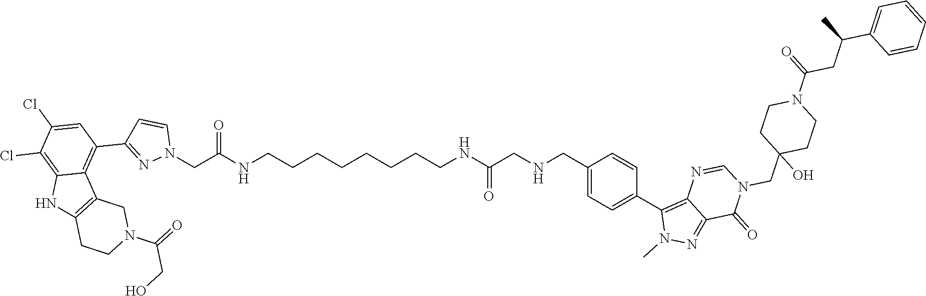 [SMILES: C[C@H](CC(=O)N1CCC(O)(Cn2cnc3c(-c4ccc(CNCC(=O)NCCCCCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (8-(2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)octyl) acetamide |
| 49 | XH168- 149 | 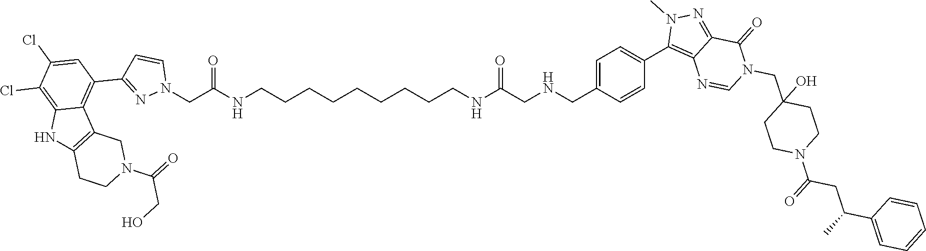 [SMILES: C[C@H](CC(=O)N1CCC(O)(Cn2cnc3c(-c4ccc(CNCC(=O)NCCCCCCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (9-(2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)nonyl) acetamide |
| 50 | XH168- 150 | 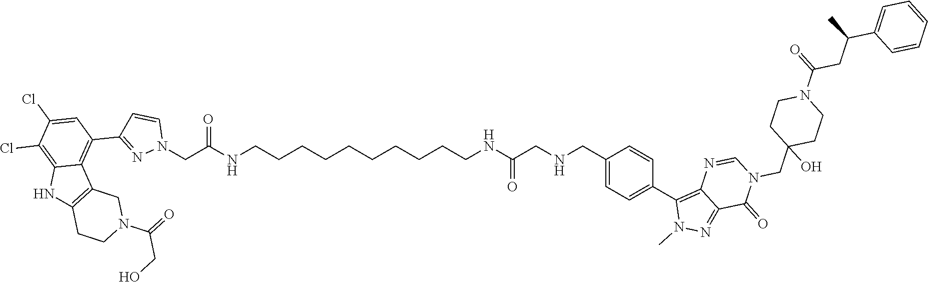 [SMILES: C[C@H](CC(=O)N1CCC(O)(Cn2cnc3c(-c4ccc(CNCC(=O)NCCCCCCCCCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (10-(2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)decyl) acetamide |
| 51 | XH168- 151 | 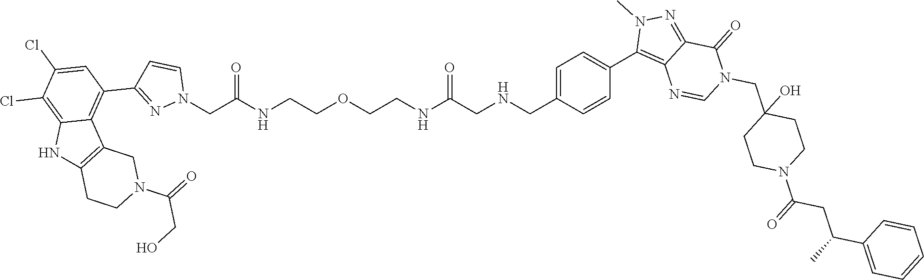 [SMILES: C[C@H](CC(=O)N1CCC(O)(Cn2cnc3c(-c4ccc(CNCC(=O)NCCOCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (2-(2-(2-((4-(6- ((4-hydroxy-1- (3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)ethoxy) ethyl)acetamide |
| 52 | XH168- 152 | 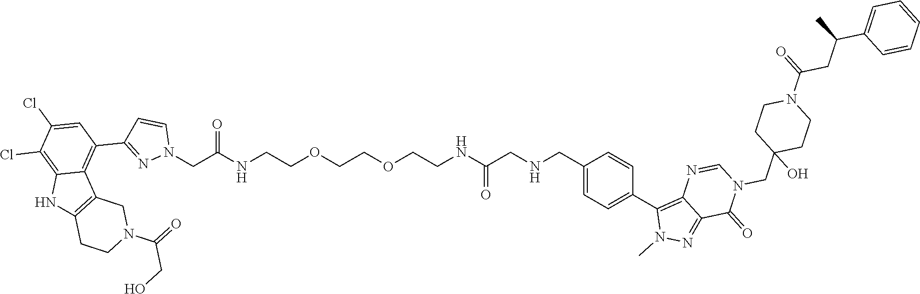 [SMILES: C[C@H](CC(=O)N1CCC(O)(Cn2cnc3c(-c4ccc(CNCC(=O)NCCOCCOCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (1-(4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)phenyl)-4- oxo-8,11-dioxa- 2,5- diazatridecan- 13-yl)acetamide |
| 53 | XH168- 153 | 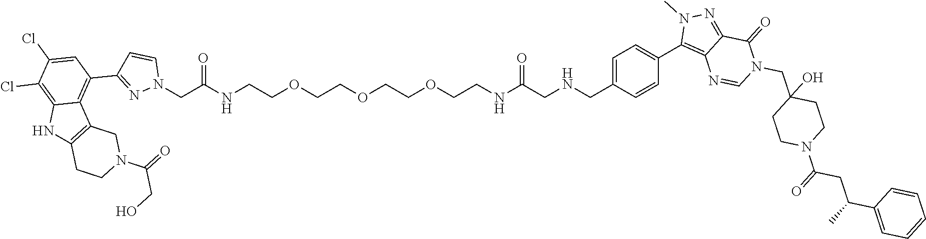 [SMILES: C[C@H](CC(=O)N1CCC(O)(Cn2cnc3c(-c4ccc(CNCC(=O)NCCOCCOCCOCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (1-(4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)phenyl)-4- oxo-8,11,14- trioxa-2,5- diazahexadecan- 16-yl)acetamide |
| 54 | XH168- 154 | 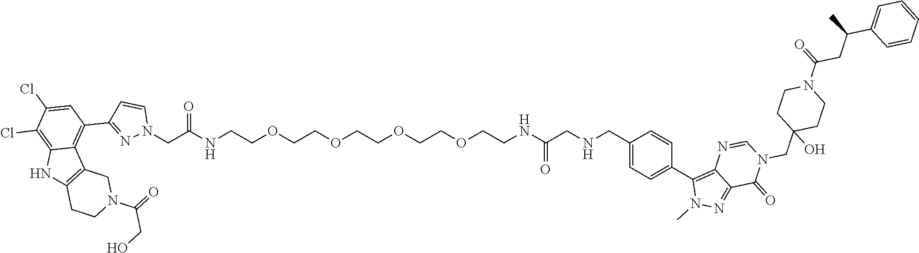 [SMILES: C[C@H](CC(=O)N1CCC(O)(Cn2cnc3c(-c4ccc(CNCC(=O)NCCOCCOCCOCCOCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (1-(4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)phenyl)-4- oxo-8,11,14,17- tetraoxa-2,5- diazanonadecan- 19-yl)acetamide |
| 55 | XH168- 155 | 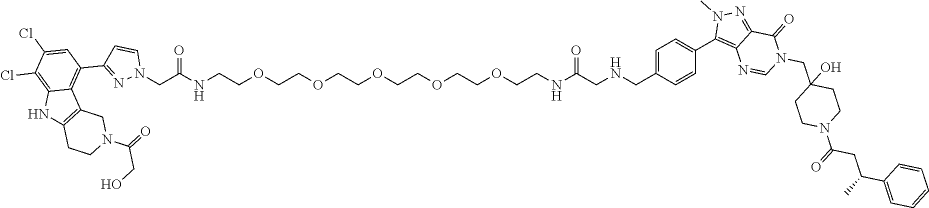 [SMILES: C[C@H](CC(=O)N1CCC(O)(Cn2cnc3c(-c4ccc(CNCC(=O)NCCOCCOCCOCCOCCOCCNC(=O)Cn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)-1H- pyrazol-1-yl)-N- (1-(4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)phenyl)-4- oxo- 8,11,14,17,20- pentaoxa-2,5- diazadocosan- 22-yl)acetamide |
| 56 | XH181- 72 | 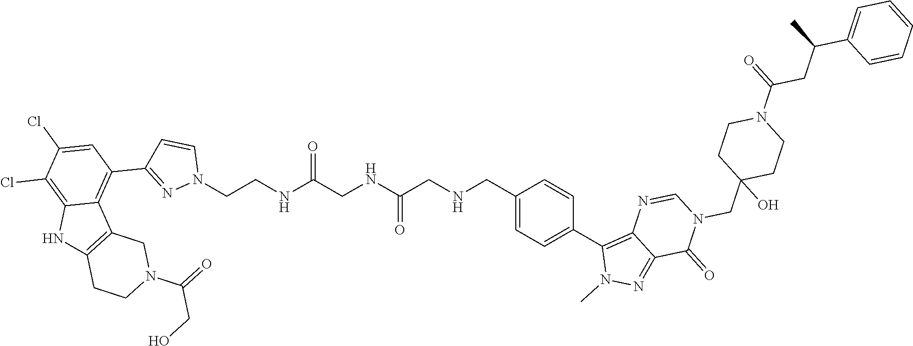 [SMILES: C[C@H](CC(=O)N1CCC(O)(Cn2cnc3c(-c4ccc(CNCC(=O)NCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-2-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido) acetamide |
| 57 | XH181- 73 | 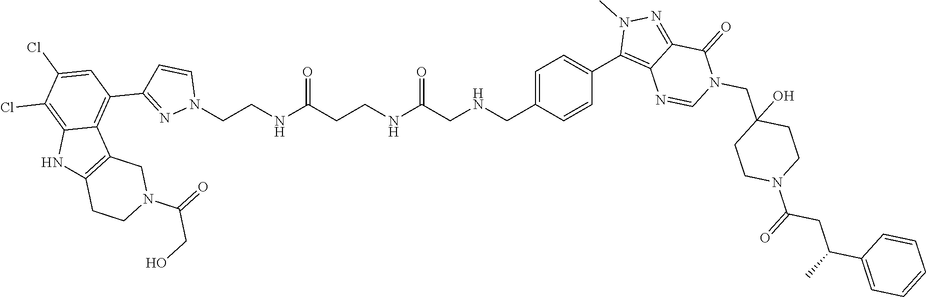 [SMILES: C[C@H](CC(=O)N1CCC(O)(Cn2cnc3c(-c4ccc(CNCC(=O)NCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-3-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido) propanamide |
| 58 | XH181- 90 | 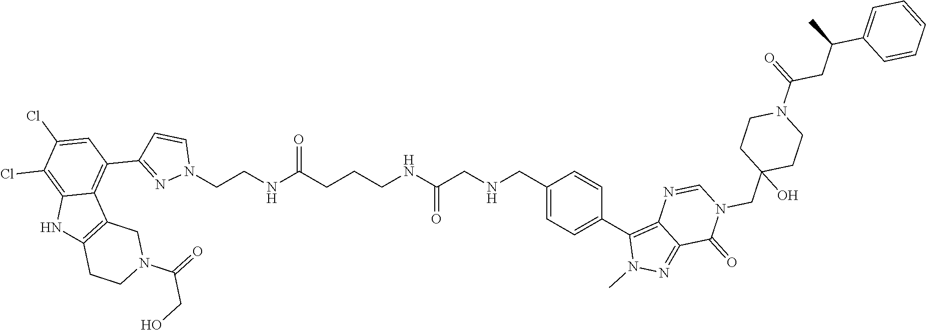 [SMILES: C[C@H](CC(=O)N1CCC(O)(Cn2cnc3c(-c4ccc(CNCC(=O)NCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-4-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido) butanamide |
| 59 | XH181- 91 | 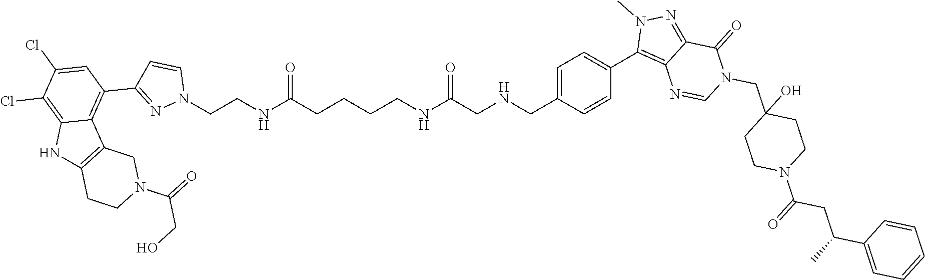 [SMILES: C[C@H](CC(=O)N1CCC(O)(Cn2cnc3c(-c4ccc(CNCC(=O)NCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-5-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido) pentanamide |
| 60 | XH181- 92 | 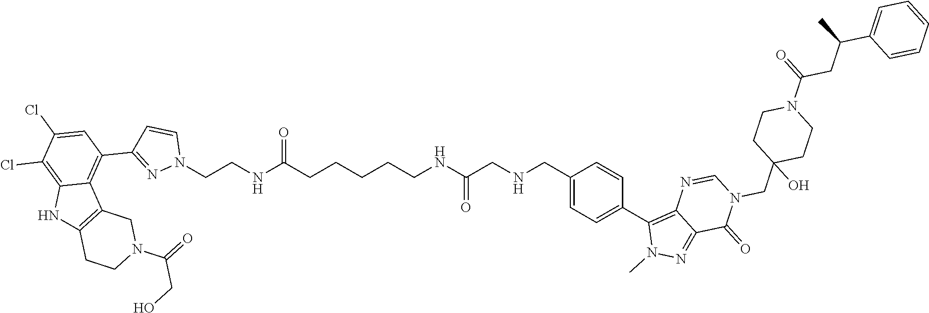 [SMILES: C[C@H](CC(=O)N1CCC(O)(Cn2cnc3c(-c4ccc(CNCC(=O)NCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-6-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido) hexanamide |
| 61 | XH181- 93 | 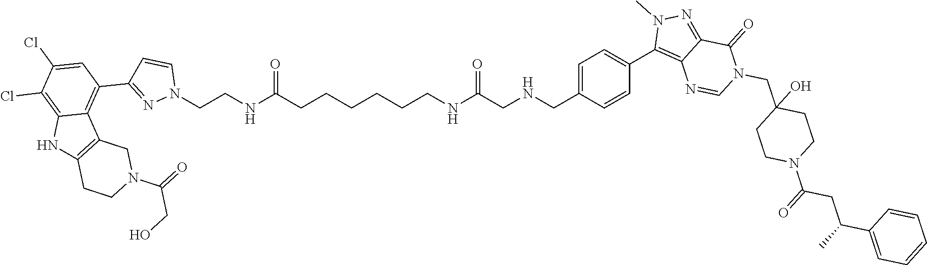 [SMILES: C[C@H](CC(=O)N1CCC(O)(Cn2cnc3c(-c4ccc(CNCC(=O)NCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-7-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido) heptanamide |
| 62 | XH181- 94 | 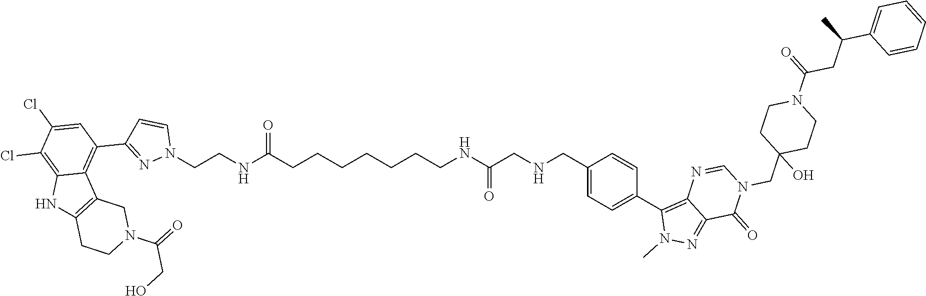 [SMILES: C[C@H](CC(=O)N1CCC(O)(Cn2cnc3c(-c4ccc(CNCC(=O)NCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-8-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido) octanamide |
| 63 | XH181- 95 | 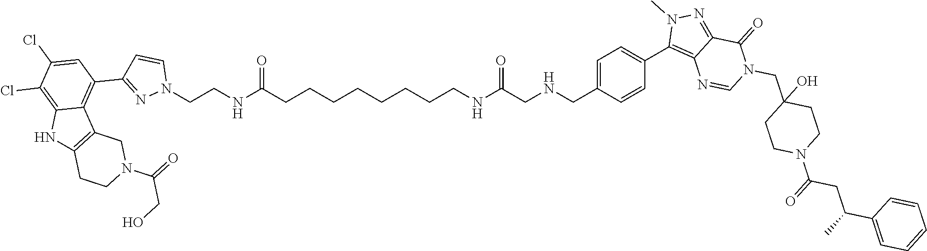 [SMILES: C[C@H](CC(=O)N1CCC(O)(Cn2cnc3c(-c4ccc(CNCC(=O)NCCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-9-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido) nonanamide |
| 64 | XH181- 96 | 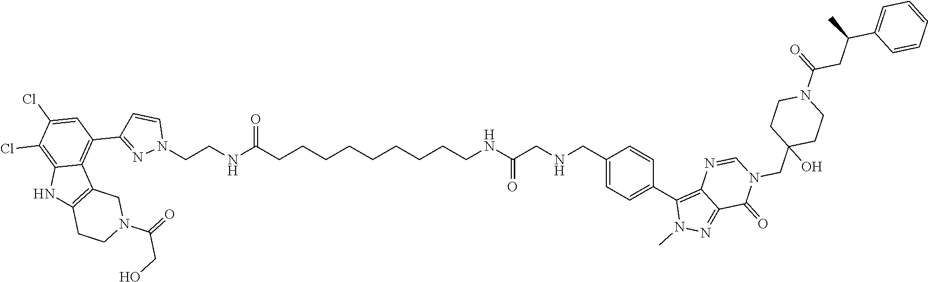 [SMILES: C[C@H](CC(=O)N1CCC(O)(Cn2cnc3c(-c4ccc(CNCC(=O)NCCCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-10-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido) decanamide |
| 65 | XH181- 97 | 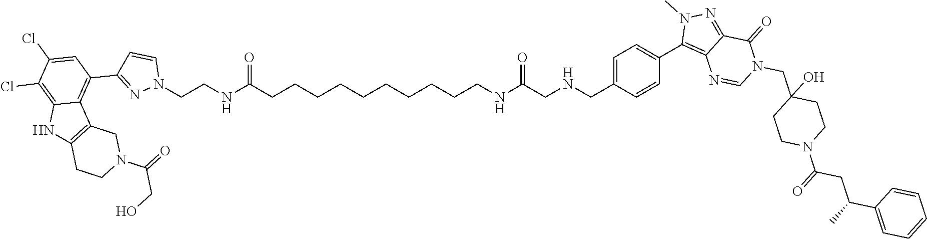 [SMILES: C[C@H](CC(=O)N1CCC(O)(Cn2cnc3c(-c4ccc(CNCC(=O)NCCCCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-11-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido) undecanamide |
| 66 | XH181- 99 | 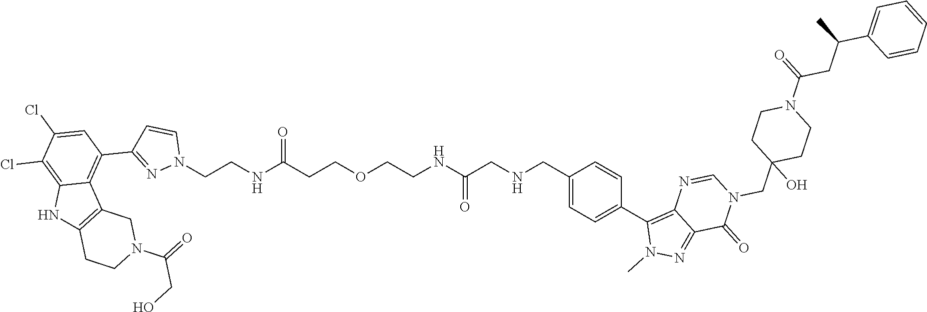 [SMILES: C[C@H](CC(=O)N1CCC(O)(Cn2cnc3c(-c4ccc(CNCC(=O)NCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-3-(2-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)ethoxy) propanamide |
| 67 | XH181- 100 | 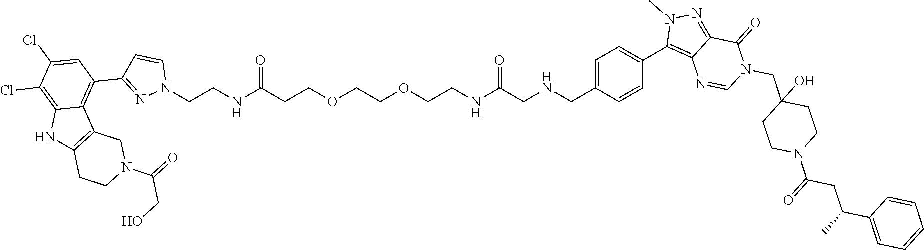 [SMILES: C[C@H](CC(=O)N1CCC(O)(Cn2cnc3c(-c4ccc(CNCC(=O)NCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-3-(2-(2- (2-((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)ethoxy) ethoxy)propan amide |
| 68 | XH181- 101 | 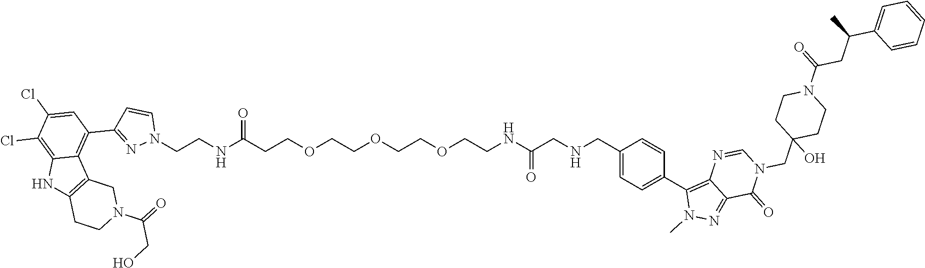 [SMILES: C[C@H](CC(=O)N1CCC(O)(Cn2cnc3c(-c4ccc(CNCC(=O)NCCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-3-((1- (4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)phenyl)-4- oxo-8,11-dioxa- 2,5- diazatridecan- 13- yl)oxy) propanamide |
| 69 | XH181- 102 | 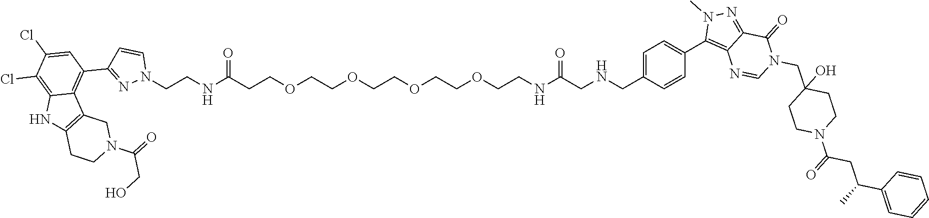 [SMILES: C[C@H](CC(=O)N1CCC(O)(Cn2cnc3c(-c4ccc(CNCC(=O)NCCOCCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-1-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)- 3,6,9,12- tetraoxapentadecan- 15-amide |
| 70 | XH181- 103 | 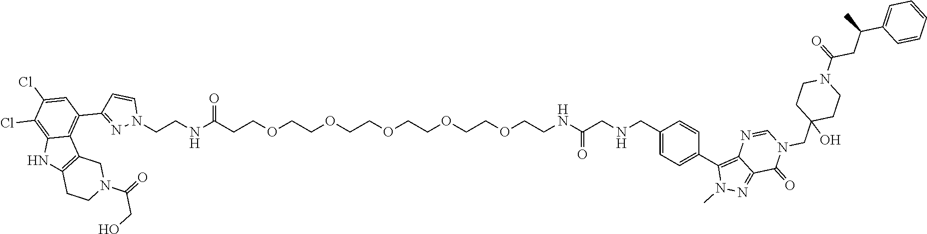 [SMILES: C[C@H](CC(=O)N1CCC(O)(Cn2cnc3c(-c4ccc(CNCC(=O)NCCOCCOCCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3-(6,7- dichloro-2-(2- hydroxyacetyl)- 2,3,4,5- tetrahydro-1H- pyrido[4,3- b]indol-9-yl)-1H- pyrazol-1- yl)ethyl)-1-(2- ((4-(6-((4- hydroxy-1-(3- phenylbutanoyl) piperidin-4- yl)methyl)-2- methyl-7-oxo- 6,7-dihydro-2H- pyrazolo[4,3- d]pyrimidin-3- yl)benzyl)amino) acetamido)- 3,6,9,12,15- pentaoxaoctadecan- 18-amide |
| 71 | XH188- 48 | 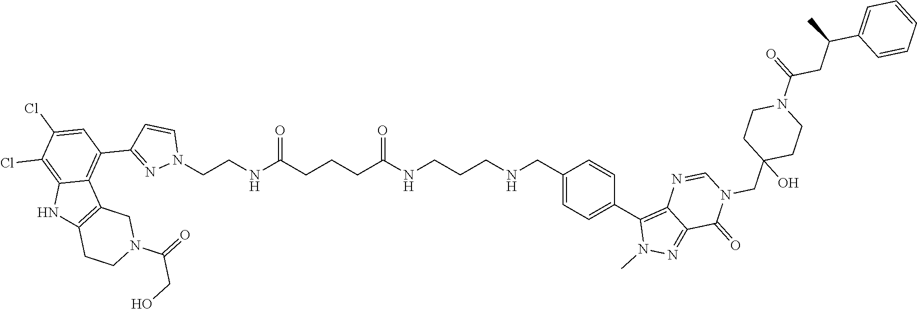 [SMILES: C[C@H](CC(=O)N1CCC(O)(Cn2cnc3c(-c4ccc(CNCCCNC(=O)CCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N1-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-N5- (3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) glutaramide |
| 72 | XH188- 49 | 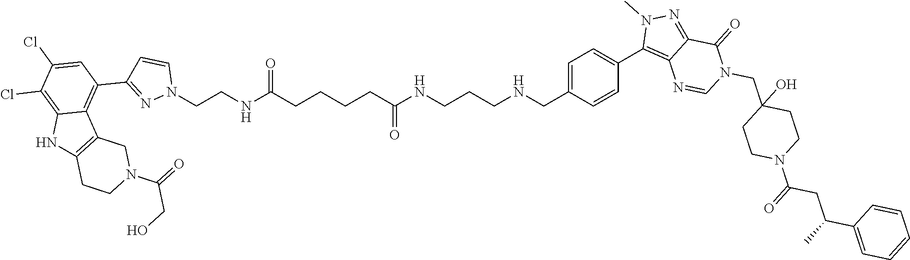 [SMILES: C[C@H](CC(=O)N1CCC(O)(Cn2cnc3c(-c4ccc(CNCCCNC(=O)CCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N1-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-N6- (3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) adipamide |
| 73 | XH188- 50 | 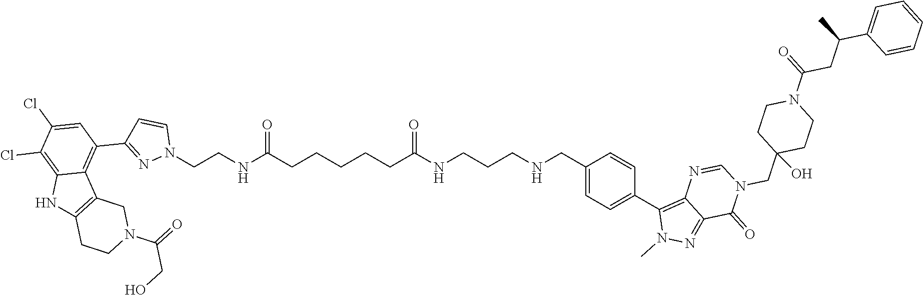 [SMILES: C[C@H](CC(=O)N1CCC(O)(Cn2cnc3c(-c4ccc(CNCCCNC(=O)CCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N1-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-N7- (3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) heptanediamide |
| 74 | XH188- 51 | 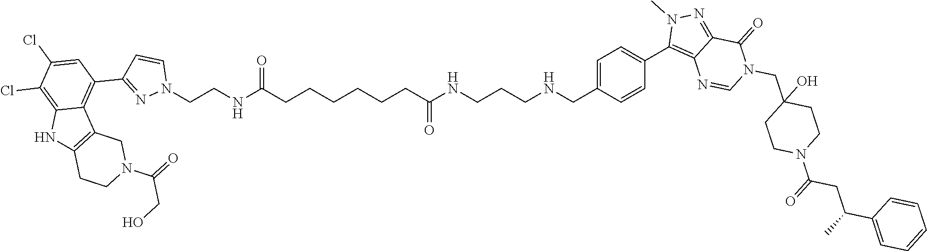 [SMILES: C[C@H](CC(=O)N1CCC(O)(Cn2cnc3c(-c4ccc(CNCCCNC(=O)CCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N1-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-N8- (3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) octanediamide |
| 75 | XH188- 52 | 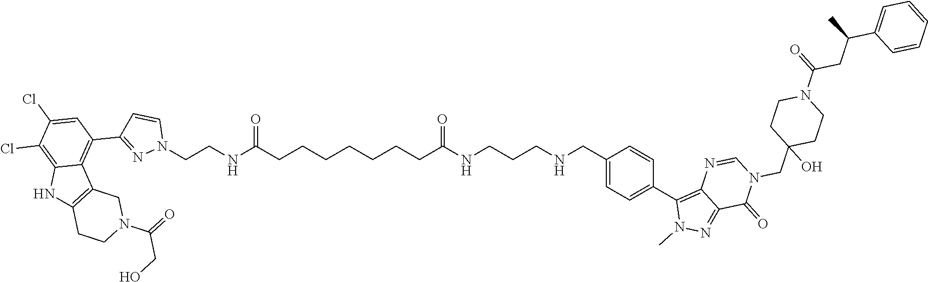 [SMILES: C[C@H](CC(=O)N1CCC(O)(Cn2cnc3c(-c4ccc(CNCCCNC(=O)CCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N1-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-N9- (3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) nonanediamide |
| 76 | XH188- 53 | 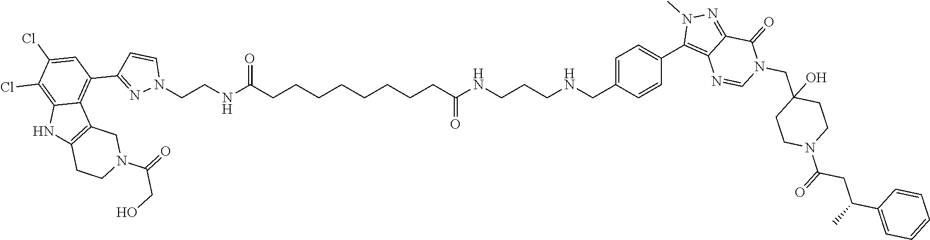 [SMILES: C[C@H](CC(=O)N1CCC(O)(Cn2cnc3c(-c4ccc(CNCCCNC(=O)CCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N1-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-N10- (3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) decanediamide |
| 77 | XH188- 54 | 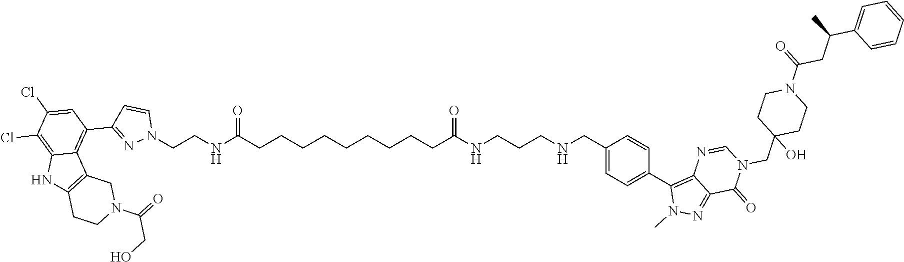 [SMILES: C[C@H](CC(=O)N1CCC(O)(Cn2cnc3c(-c4ccc(CNCCCNC(=O)CCCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N1-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-N11- (3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) undecanediamide |
| 78 | XH188- 55 | 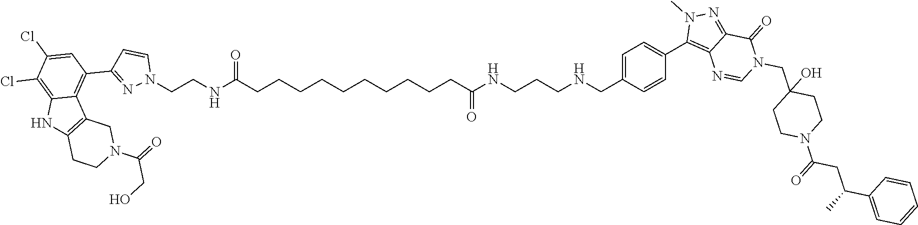 [SMILES: C[C@H](CC(=O)N1CCC(O)(Cn2cnc3c(-c4ccc(CNCCCNC(=O)CCCCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N1-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-N12- (3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) dodecanediamide |
| 79 | XH188- 56 | 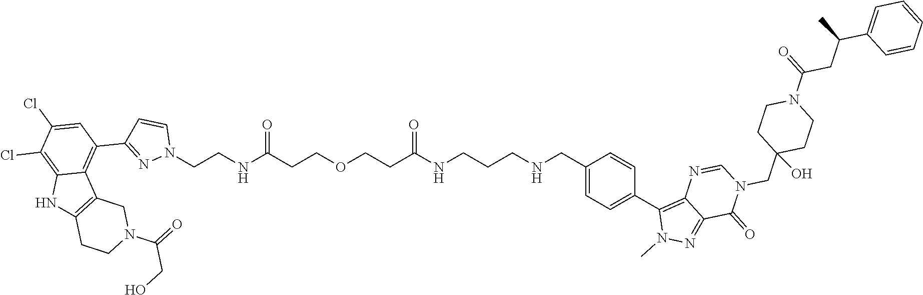 [SMILES: C[C@H](CC(=O)N1CCC(O)(Cn2cnc3c(-c4ccc(CNCCCNC(=O)CCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-3-(3- ((3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl)amino)- 3- oxopropoxy) propenamide |
| 80 | XH188- 57 | 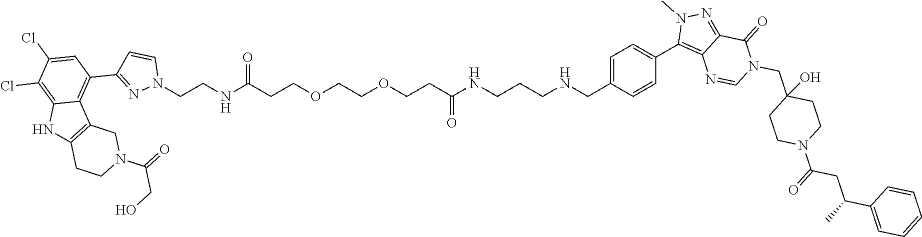 [SMILES: C[C@H](CC(=O)N1CCC(O)(Cn2cnc3c(-c4ccc(CNCCCNC(=O)CCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-3-(2- (3-((3-((4-(6- ((4-hydroxy-1- (3- phenylbutanoyl) piperidin-4- yl)methyl)-2- methyl-7-oxo- 6,7-dihydro- 2H- pyrazolo[4,3- d]pyrimidin-3- yl)benzyl)amino) propyl)amino)- 3- oxopropoxy) ethoxy) propenamide |
| 81 | XH188- 58 | 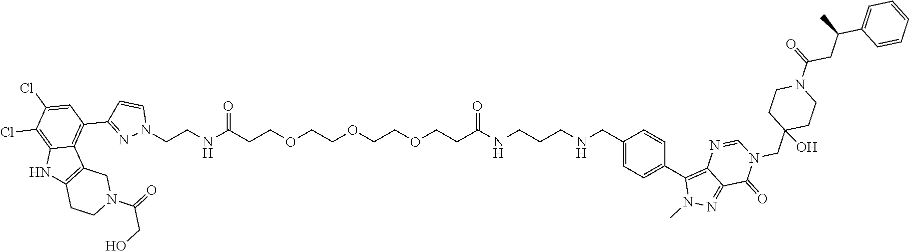 [SMILES: C[C@H](CC(=O)N1CCC(O)(Cn2cnc3c(-c4ccc(CNCCCNC(=O)CCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-3-((1- (4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)phenyl)-7- oxo-10,13- dioxa-2,6- diazapentadecan- 15- yl)oxy) propenamide |
| 82 | XH188- 59 | 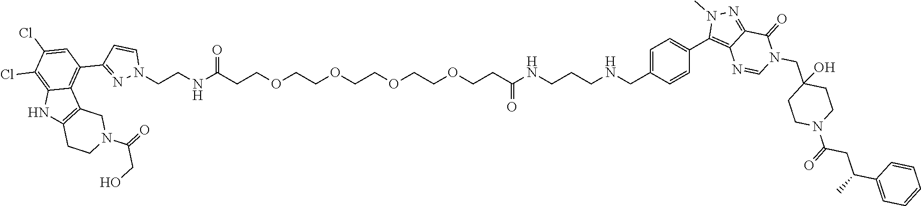 [SMILES: C[C@H](CC(=O)N1CCC(O)(Cn2cnc3c(-c4ccc(CNCCCNC(=O)CCOCCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N1-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-N16- (3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl)- 4,7,10,13- tetraoxahexade canediamide |
| 83 | XH188- 60 | 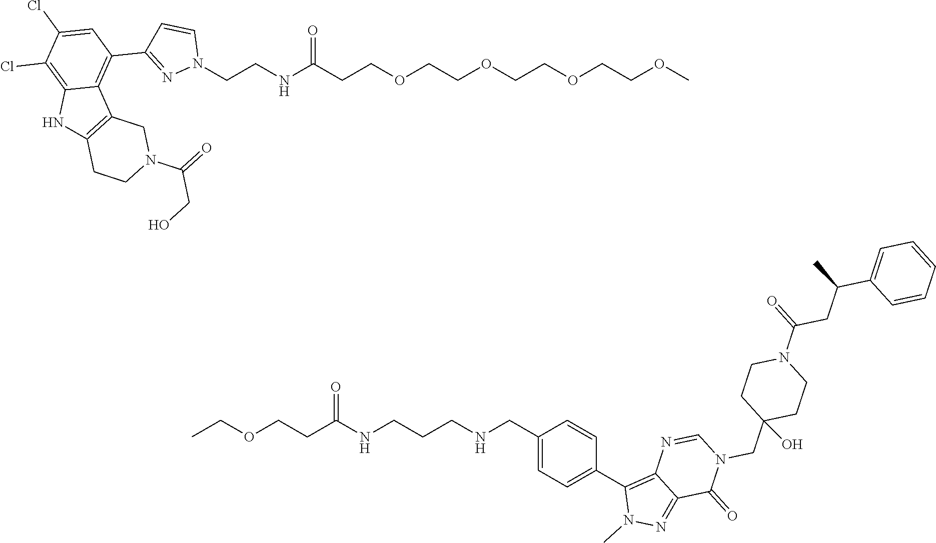 | (R)-N¹-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-N¹⁹-_ (3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl)- 4,7,10,13,16- pentaoxa- nonadecanediamide |
| 84 | XH188- 90 | 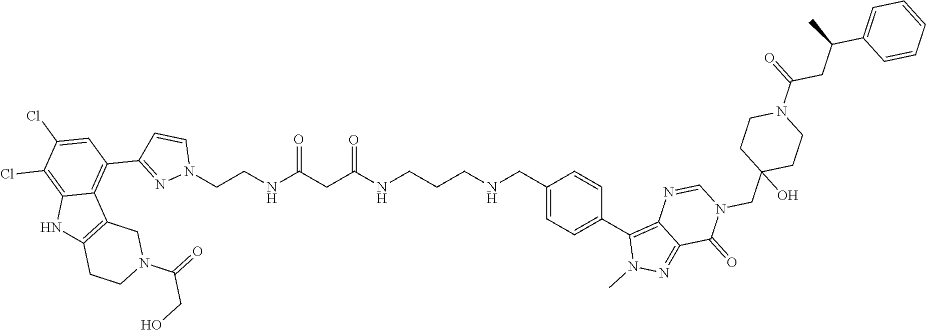 [SMILES: C[C@H](CC(=O)N1CCC(O)(Cn2cnc3c(-c4ccc(CNCCCNC(=O)CC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-2-(3-(6,7- dichloro-2-(2- hydroxyacetyl)- 2,3,4,5- tetrahydro-1H- pyrido[4,3- b]indol-9-yl)- 1H-pyrazol-1- yl)-N-(2-((3- ((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl)amino)- 2- oxoethyl) acetamide |
| 85 | XH188- 91 | 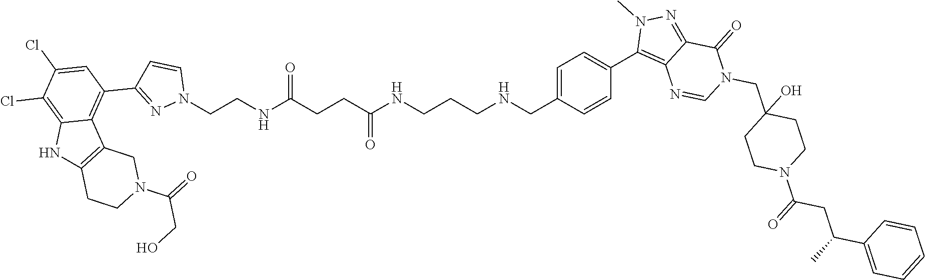 [SMILES: C[C@H](CC(=O)N1CCC(O)(Cn2cnc3c(-c4ccc(CNCCCNC(=O)CCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-3-(2-(3- (6,7-dichloro- 2-(2- hydroxyacetyl)- 2,3,4,5- tetrahydro-1H- pyrido[4,3- b]indol-9-yl)- 1H-pyrazol-1- yl)acetamido)- N-(3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) propanamide |
| 86 | XH188- 92 | 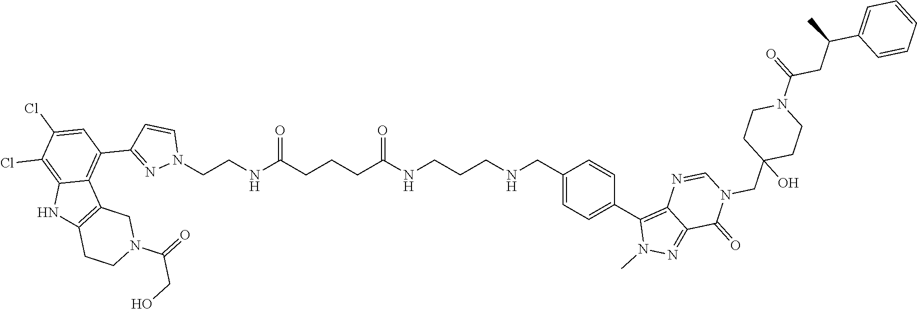 [SMILES: C[C@H](CC(=O)N1CCC(O)(Cn2cnc3c(-c4ccc(CNCCCNC(=O)CCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-4-(2-(3- (6,7-dichloro- 2-(2- hydroxyacetyl)- 2,3,4,5- tetrahydro-1H- pyrido[4,3- b]indol-9-yl)- 1H-pyrazol-1- yl)acetamido)- N-(3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) butanamide |
| 87 | XH188- 93 | 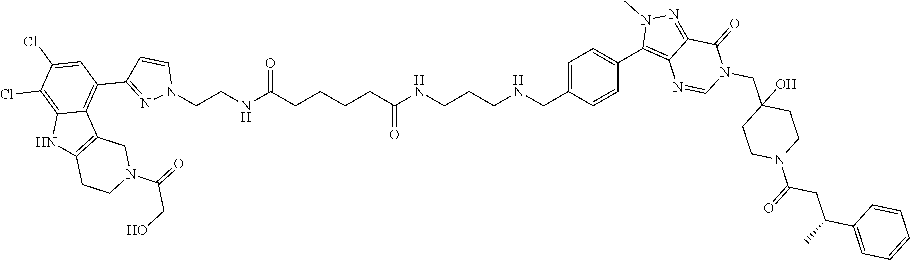 [SMILES: C[C@H](CC(=O)N1CCC(O)(Cn2cnc3c(-c4ccc(CNCCCNC(=O)CCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-5-(2-(3- (6,7-dichloro- 2-(2- hydroxyacetyl)- 2,3,4,5- tetrahydro-1H- pyrido[4,3- b]indol-9-yl)- 1H-pyrazol-1- yl)acetamido)- N-(3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) pentanamide |
| 88 | XH188- 94 | 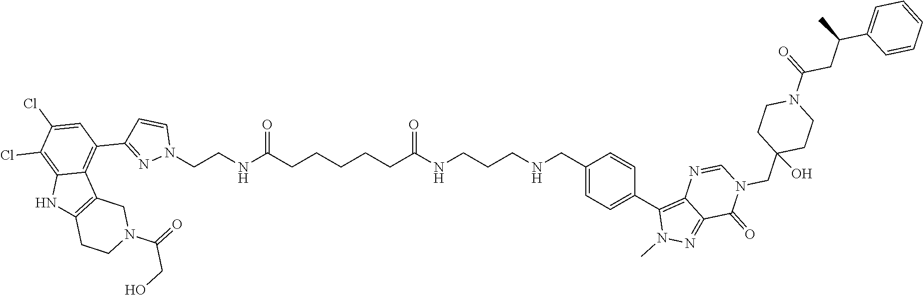 [SMILES: C[C@H](CC(=O)N1CCC(O)(Cn2cnc3c(-c4ccc(CNCCCNC(=O)CCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-6-(2-(3- (6,7-dichloro- 2-(2- hydroxyacetyl)- 2,3,4,5- tetrahydro-1H- pyrido[4,3- b]indol-9-yl)- 1H-pyrazol-1- yl)acetamido)- N-(3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) hexanamide |
| 89 | XH188- 95 | 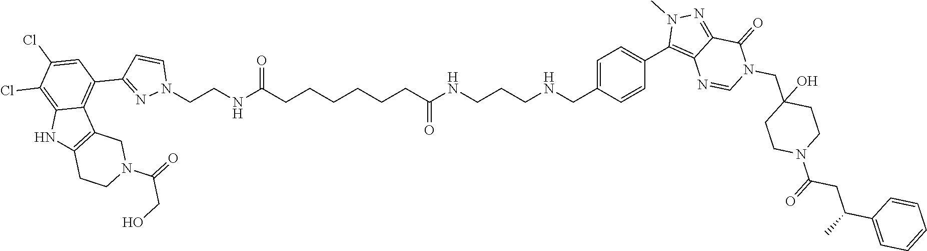 [SMILES: C[C@H](CC(=O)N1CCC(O)(Cn2cnc3c(-c4ccc(CNCCCNC(=O)CCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-7-(2-(3- (6,7-dichloro- 2-(2- hydroxyacetyl)- 2,3,4,5- tetrahydro-1H- pyrido[4,3- b]indol-9-yl)- 1H-pyrazol-1- yl)acetamido)- N-(3-((4-(6-((4- hydroxy-1-(3- phenylbutanoyl) piperidin-4- yl)methyl)-2- methyl-7-oxo- 6,7-dihydro- 2H- pyrazolo[4,3- djpyrimidin-3- yl)benzyl)amino) propyl) heptanamide |
| 90 | XH188- 96 | 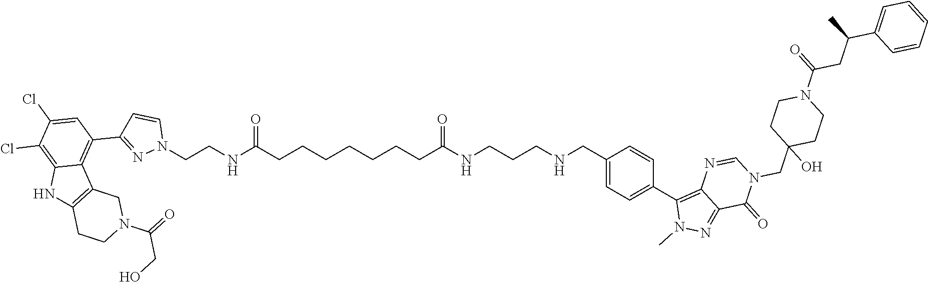 [SMILES: C[C@H](CC(=O)N1CCC(O)(Cn2cnc3c(-c4ccc(CNCCCNC(=O)CCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-8-(2-(3- (6,7-dichloro- 2-(2- hydroxyacetyl)- 2,3,4,5- tetrahydro-1H- pyrido[4,3- b]indol-9-yl)- 1H-pyrazol-1- yl)acetamido)- N-(3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) octanamide |
| 91 | XH188- 97 | 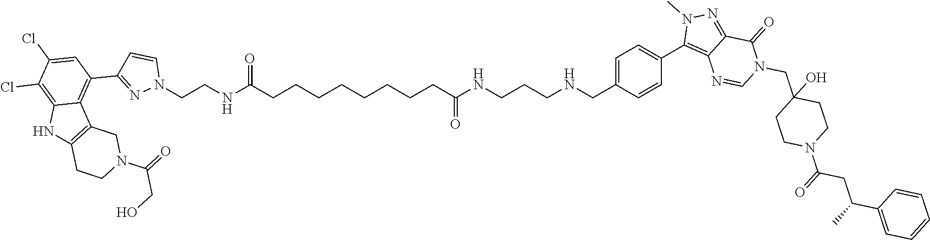 [SMILES: C[C@H](CC(=O)N1CCC(O)(Cn2cnc3c(-c4ccc(CNCCCNC(=O)CCCCCCCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-9-(2-(3- (6,7-dichloro- 2-(2- hydroxyacetyl)- 2,3,4,5- tetrahydro-1H- pyrido[4,3- b]indol-9-yl)- 1H-pyrazol-1- yl)acetamido)- N-(3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) nonanamide |
| 92 | XH188- 98 | 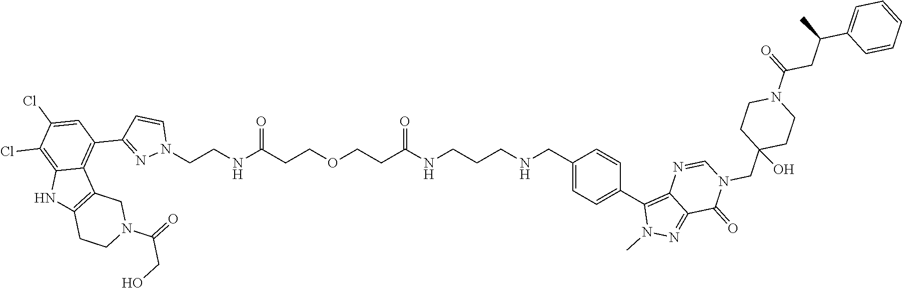 [SMILES: C[C@H](CC(=O)N1CCC(O)(Cn2cnc3c(-c4ccc(CNCCCNC(=O)CCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-N-(2-(3- (6,7-dichloro- 2-(2- hydroxyacetyl)- 2,3,4,5- tetrahydro-1H- pyrido[4,3- b]indol-9-yl)- 1H-pyrazol-1- yl)ethyl)-3-(3- ((3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl)amino)- 3- oxopropoxy) propanamide |
| 93 | XH188- 99 | 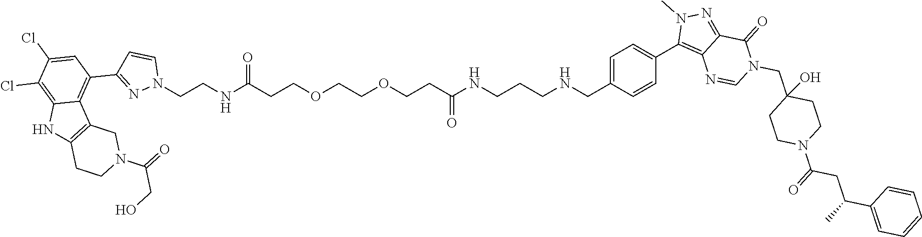 [SMILES: C[C@H](CC(=O)N1CCC(O)(Cn2cnc3c(-c4ccc(CNCCCNC(=O)CCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-3-(2-(2-(3- (6,7-dichloro- 2-(2- hydroxyacetyl)- 2,3,4,5- tetrahydro-1H- pyrido[4,3- b]indol-9-yl)- 1H-pyrazol-1- yl)acetamido) ethoxy)-N-(3- ((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) propanamide |
| 94 | XH188- 100 | 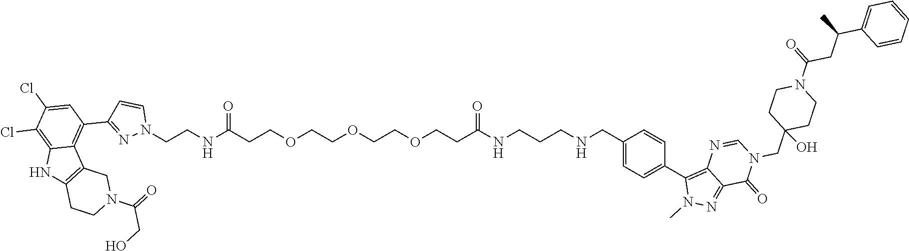 [SMILES: C[C@H](CC(=O)N1CCC(O)(Cn2cnc3c(-c4ccc(CNCCCNC(=O)CCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-3-(2-(2-(2- (2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) ethoxy)ethoxy) ethoxy)-N-(3- ((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl) propanamide |
| 95 | XH188- 101 | 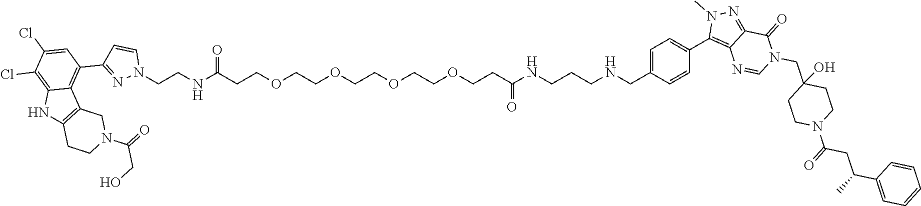 [SMILES: C[C@H](CC(=O)N1CCC(O)(Cn2cnc3c(-c4ccc(CNCCCNC(=O)CCOCCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-1-(2-(3- (6,7-dichloro- 2-(2- hydroxyacetyl)- 2,3,4,5- tetrahydro-1H- pyrido[4,3- b]indol-9-yl)- 1H-pyrazol-1- yl)acetamido)- N-(3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl)- 3,6,9,12- tetraoxapentadecan- 15-amide |
| 96 | XH188- 102 | 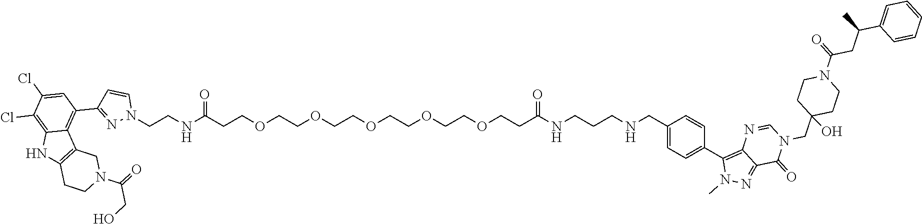 [SMILES: C[C@H](CC(=O)N1CCC(O)(Cn2cnc3c(-c4ccc(CNCCCNC(=O)CCOCCOCCOCCOCCOCCC(=O)NCCn5ccc(-c6cc(Cl)c(Cl)c7[nH]c8c(c67)CN(C(=O)CO)CC8)n5)cc4)n(C)nc3c2=O)CC1)c1ccccc1] | (R)-1-(2-(3- (6,7-dichloro- 2-(2- hydroxyacetyl)- 2,3,4,5- tetrahydro-1H- pyrido[4,3- b]indol-9-yl)- 1H-pyrazol-1- yl)acetamido)- N-(3-((4-(6-((4- hydroxy-1-(3- phenylbutanoyl) piperidin-4- yl)methyl)-2- methyl-7-oxo- 6,7-dihydro- 2H- pyrazolo[4,3- d]pyrimidin-3- yl)benzyl)amino) propyl)- 3,6,9,12,15- pentaoxaoctadecan- 18-amide |
| 97 | KL186- 107 | 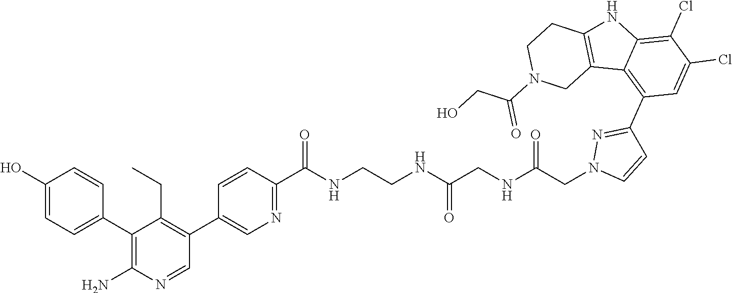 [SMILES: CCc1c(-c2ccc(C(=O)NCCNC(=O)CNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(2- (2-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) acetamido)ethyl)- 4′-ethyl-5′-(4- hydroxyphenyl)- [3,3′- bipyridine]-6- carboxamide |
| 98 | KL186- 115 | 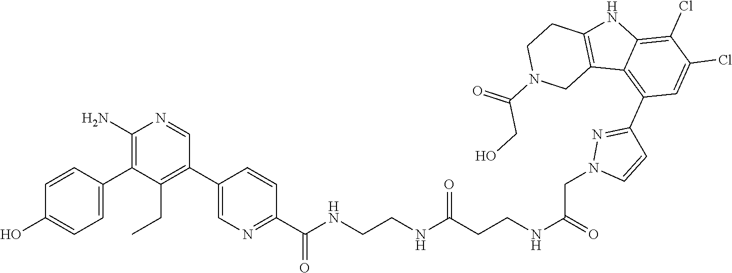 [SMILES: CCc1c(-c2ccc(C(=O)NCCNC(=O)CCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(2- (3-(2-(3-(6,7- dichloro-2-(2- hydroxyacetyl)- 2,3,4,5- tetrahydro-1H- pyrido[4,3- b]indol-9-yl)-1H- pyrazol-1- yl)acetamido) propane- mido)ethyl)-4′- ethyl-5′-(4- hydroxyphenyl)- [3,3′- bipyridine]-6- carboxamide |
| 99 | KL186- 116 | 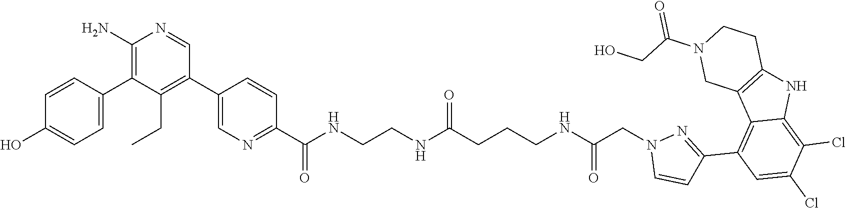 [SMILES: CCc1c(-c2ccc(C(=O)NCCNC(=O)CCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(2- (4-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) butanamido)- ethyl)-4′-ethyl- 5′-(4- hydroxyphenyl)- [3,3′- bipyridine]-6- carboxamide |
| 100 | KL186- 117 | 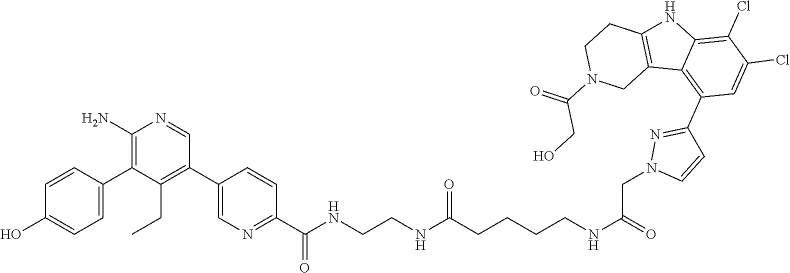 [SMILES: CCc1c(-c2ccc(C(=O)NCCNC(=O)CCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(2- (5-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido)- pentan- amido)ethyl)-4′- ethyl-5′-(4- hydroxyphenyl)- [3,3′- bipyridine]-6- carboxamide |
| 101 | KL186- 118 | 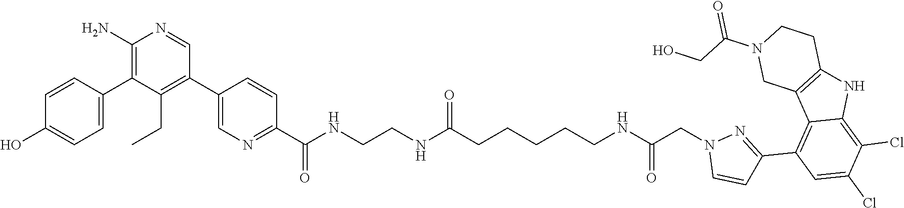 [SMILES: CCc1c(-c2ccc(C(=O)NCCNC(=O)CCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(2- (6-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) hexan- amido)ethyl)-4′- ethyl-5′-(4- hydroxyphenyl)- [3,3′- bipyridine]-6- carboxamide |
| 102 | KL186- 119 | 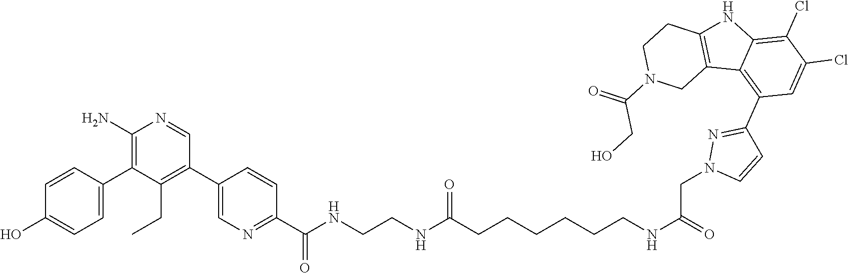 [SMILES: CCc1c(-c2ccc(C(=O)NCCNC(=O)CCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(2- (7-(2-(3-(6,7- dichloro-2-(2- hydroxyacetyl)- 2,3,4,5- tetrahydro-1H- pyrido[4,3- b]indol-9-yl)-1H- pyrazol-1- yl)acetamido) heptan- amido)ethyl)-4′- ethyl-5′-(4- hydroxyphenyl)- [3,3′- bipyridine]-6- carboxamide |
| 103 | KL186- 120 | 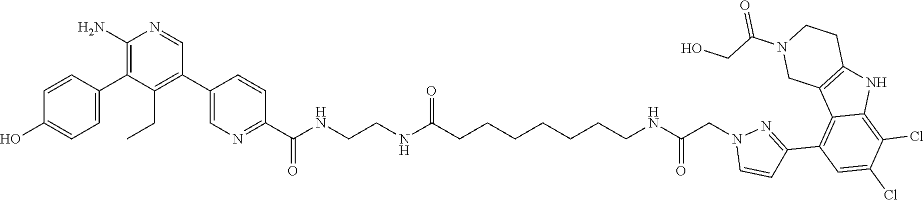 [SMILES: CCc1c(-c2ccc(C(=O)NCCNC(=O)CCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(2- (8-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) octanamido)- ethyl)-4′-ethyl- 5′-(4- hydroxyphenyl)- [3,3′- bipyridine]-6- carboxamide |
| 104 | KL186- 121 | 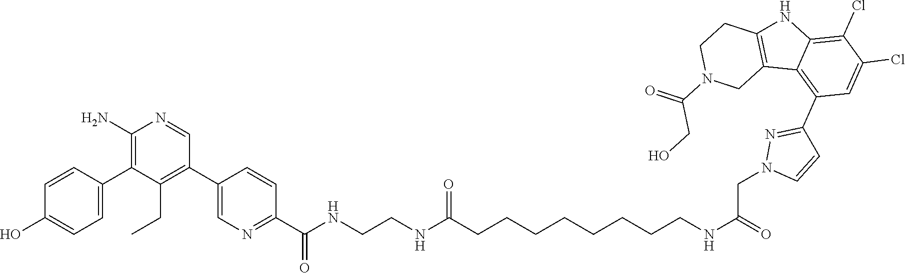 [SMILES: CCc1c(-c2ccc(C(=O)NCCNC(=O)CCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(2- (9-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) nonan- amido)ethyl)-4′- ethyl-5′-(4- hydroxyphenyl)- [3,3′- bipyridine]-6- carboxamide |
| 105 | KL186- 122 | 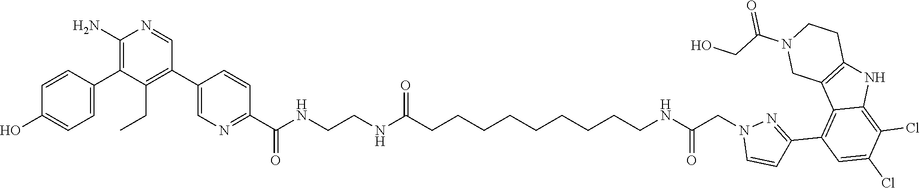 [SMILES: CCc1c(-c2ccc(C(=O)NCCNC(=O)CCCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(2- (10-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) decan- amido)ethyl)-4′- ethyl-5′-(4- hydroxyphenyl)- [3,3′- bipyridine]-6- carboxamide |
| 106 | KL186- 123 | 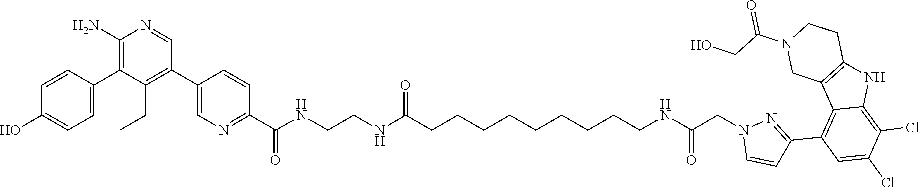 [SMILES: CCc1c(-c2ccc(C(=O)NCCNC(=O)CCCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(2- (11-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) undecane- amido)ethyl)-4′- ethyl-5′-(4- hydroxyphenyl)- [3,3′- bipyridine]-6- carboxamide |
| 107 | KL186- 124 | 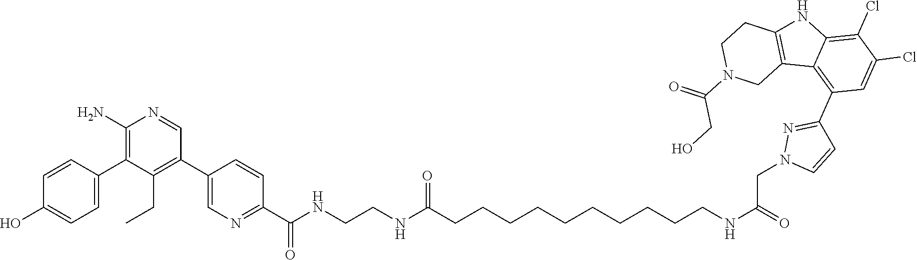 [SMILES: CCc1c(-c2ccc(C(=O)NCCNC(=O)CCCCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(2- (3-(2-(2-(3- (6,7-dichloro- 2-(2- hydroxyacetyl)- 2,3,4,5- tetrahydro-1H- pyrido[4,3- b]indol-9-yl)- 1H-pyrazol-1- yl)acetamido) ethoxy)pro- panamido)ethyl)- 4′-ethyl-5′-(4- hydroxyphenyl)- [3,3′- bipyridine]-6- carboxamide |
| 108 | KL186- 125 | 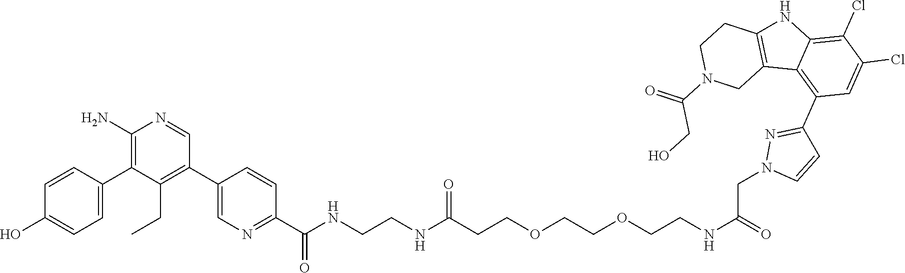 [SMILES: CCc1c(-c2ccc(C(=O)NCCNC(=O)CCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(1- (3-(6,7-dichloro- 2-(2- hydroxyacetyl)- 2,3,4,5- tetrahydro-1H- pyrido[4,3- b]indol-9-yl)-1H- pyrazol-1-yl)- 2,12-dioxo-6,9- dioxa-3,13- diazapentadecan- 15-yl)-4′-ethyl- 5′-(4- hydroxyphenyl)- [3,3′- bipyridine]-6- carboxamide |
| 109 | KL186- 126 | 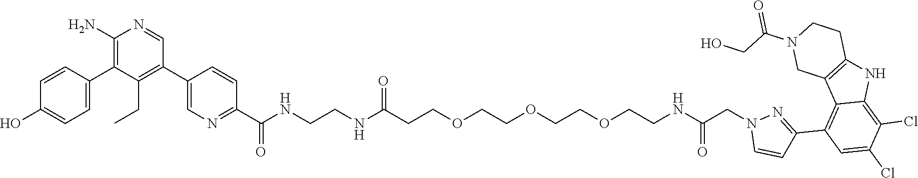 [SMILES: CCc1c(-c2ccc(C(=O)NCCNC(=O)CCOCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(1- (3-(6,7-dichloro- 2-(2- hydroxyacetyl)- 2,3,4,5- tetrahydro-1H- pyrido[4,3- b]indol-9-yl)-1H- pyrazol-1-yl)- 2,15-dioxo- 6,9,12-trioxa- 3,16- diazaoctadecan- 18-yl)-4′-ethyl- 5′-(4- hydroxyphenyl)- [3,3′- bipyridine]-6- carboxamide |
| 110 | KL186- 127 | 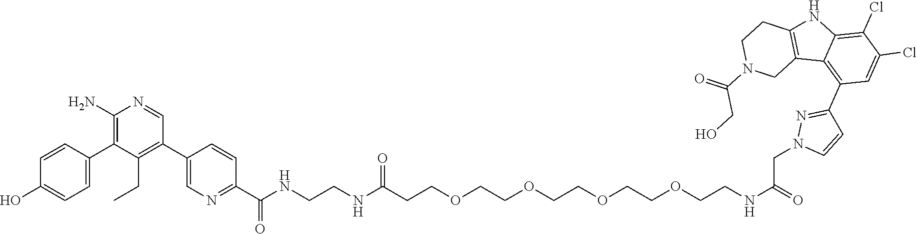 [SMILES: CCc1c(-c2ccc(C(=O)NCCNC(=O)CCOCCOCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(1- (3-(6,7-dichloro- 2-(2- hydroxyacetyl)- 2,3,4,5- tetrahydro-1H- pyrido[4,3- b]indol-9-yl)-1H- pyrazol-1-yl)- 2,18-dioxo- 6,9,12,15- tetraoxa-3,19- diazahenicosan- 21-yl)-4′-ethyl- 5′-(4- hydroxyphenyl)- [3,3′- bipyridine]-6- carboxamide |
| 111 | KL186- 128 | 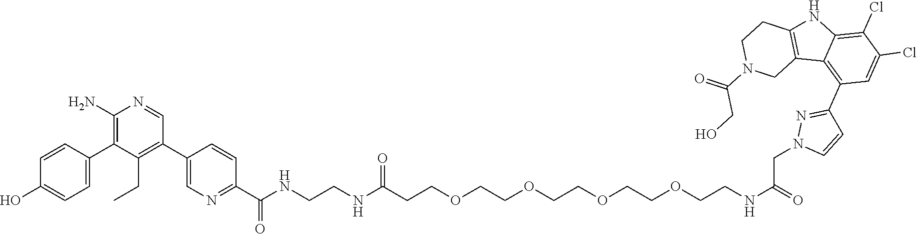 [SMILES: CCc1c(-c2ccc(C(=O)NCCNC(=O)CCOCCOCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(1- (3-(6,7-dichloro- 2-(2- hydroxyacetyl)- 2,3,4,5- tetrahydro-1H- pyrido[4,3- b]indol-9-yl)-1H- pyrazol-1-yl)- 2,21-dioxo- 6,9,12,15,18- pentaoxa-3,22- diazatetracosan- 24-yl)-4′-ethyl- 5′-(4- hydroxyphenyl)- [3,3′- bipyridine]-6- carboxamide |
| 112 | KL186- 129 | 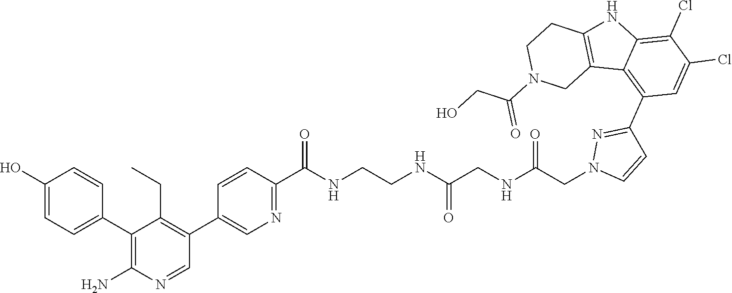 [SMILES: CCc1c(-c2ccc(C(=O)NCCNC(=O)CNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((2-(2-(3-(6,7- dichloro-2-(2- hydroxyacetyl)- 2,3,4,5- tetrahydro-1H- pyrido[4,3- b]indol-9-yl)-1H- pyrazol-1- yl)acetamido) ethyl)amino)-3- oxopropyl)-4′- ethyl-5′-(4- hydroxyphenyl)- [3,3′- bipyridine]-6- carboxamide |
| 113 | KL186- 130 | 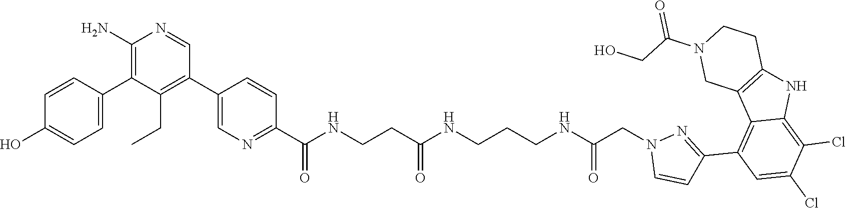 [SMILES: CCc1c(-c2ccc(C(=O)NCCC(=O)NCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((3-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) propyl)- amino)-3- oxopropyl)-4′- ethyl-5′-(4- hydroxyphenyl)- [3,3′- bipyridine]-6- carboxamide |
| 114 | KL186- 131 | 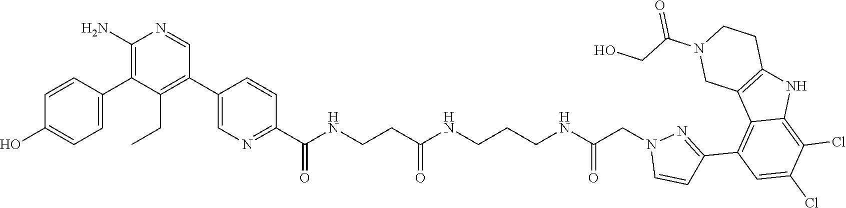 [SMILES: CCc1c(-c2ccc(C(=O)NCCC(=O)NCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((4-(2-(3-(6,7- dichloro-2-(2- hydroxyacetyl)- 2,3,4,5- tetrahydro-1H- pyrido[4,3- b]indol-9-yl)-1H- pyrazol-1- yl)acetamido) butyl)-amino)-3- oxopropyl)-4′- ethyl-5′-(4- hydroxyphenyl)- [3,3′- bipyridine]-6- carboxamide |
| 115 | KL186- 132 | 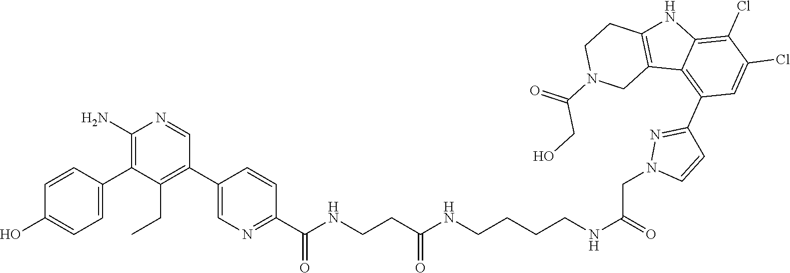 [SMILES: CCc1c(-c2ccc(C(=O)NCCC(=O)NCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((5-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) pentyl)- amino)-3- oxopropyl)-4′- ethyl-5′-(4- hydroxyphenyl)- [3,3′- bipyridine]-6- carboxamide |
| 116 | KL186- 133 | 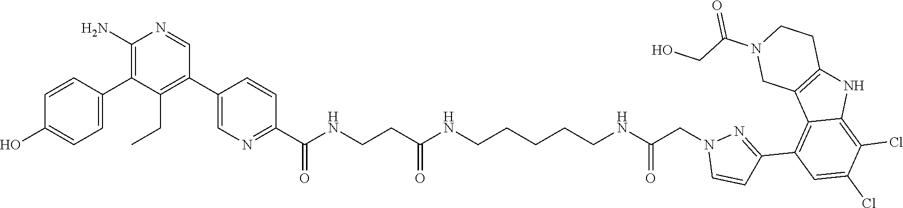 [SMILES: CCc1c(-c2ccc(C(=O)NCCC(=O)NCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((6-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) hexyl)- amino)-3- oxopropyl)-4′- ethyl-5′-(4- hydroxyphenyl)- [3,3′- bipyridine]-6- carboxamide |
| 117 | KL186- 134 | 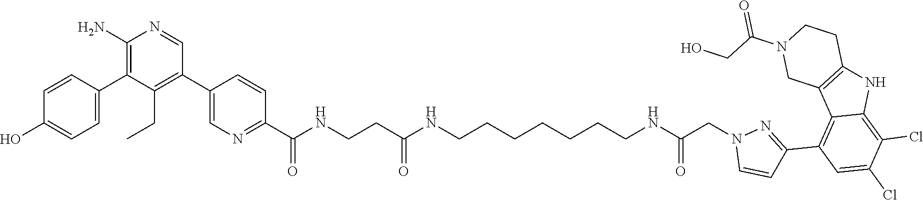 [SMILES: CCc1c(-c2ccc(C(=O)NCCC(=O)NCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((7-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) heptyl)amino)- 3-oxopropyl)- 4′-ethyl-5′-(4- hydroxyphenyl)- [3,3′- bipyridine]-6- carboxamide |
| 118 | KL186- 135 | 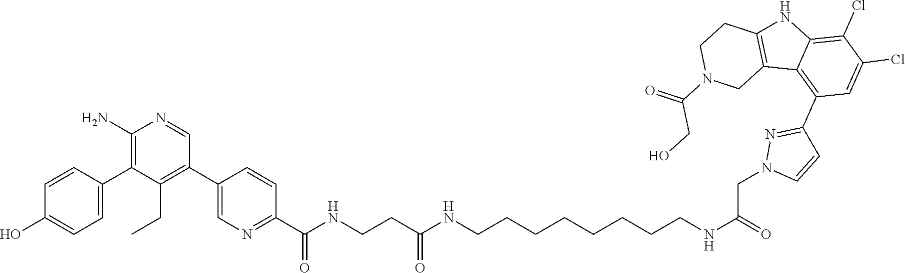 [SMILES: CCc1c(-c2ccc(C(=O)NCCC(=O)NCCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((8-(2-(3-(6,7- dichloro-2-(2- hydroxyacetyl)- 2,3,4,5- tetrahydro-1H- pyrido[4,3- b]indol-9-yl)-1H- pyrazol-1- yl)acetamido) octyl)amino)-3- oxopropyl)-4′- ethyl-5′-(4- hydroxyphenyl)- [3,3′- bipyridine]-6- carboxamide |
| 119 | KL186- 136 | 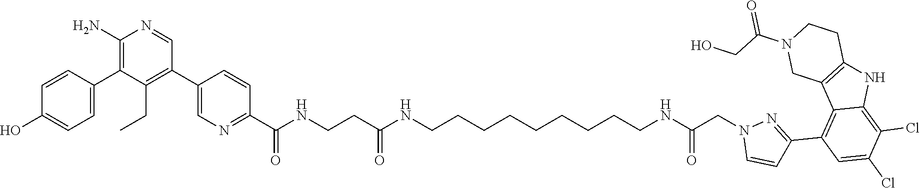 [SMILES: CCc1c(-c2ccc(C(=O)NCCC(=O)NCCCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((9-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) nonyl)amino)- 3-oxopropyl)- 4′-ethyl-5′-(4- hydroxyphenyl)- [3,3′- bipyridine]-6- carboxamide |
| 120 | KL186- 137 | 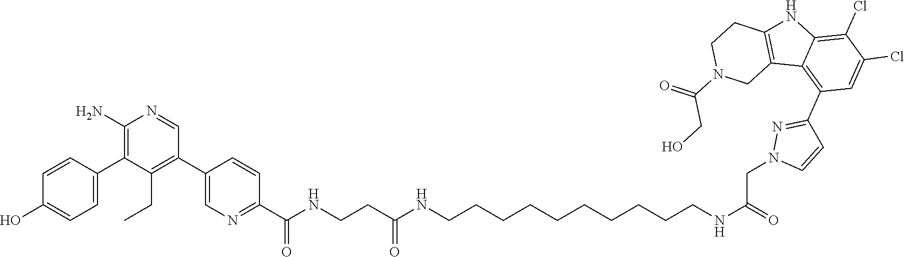 [SMILES: CCc1c(-c2ccc(C(=O)NCCC(=O)NCCCCCCCCCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((10-(2-(3-(6,7- dichloro-2-(2- hydroxyacetyl)- 2,3,4,5- tetrahydro-1H- pyrido[4,3- b]indol-9-yl)- 1H-pyrazol-1- yl)acetamido) decyl)amino)- 3-oxopropyl)- 4′-ethyl-5′-(4- hydroxyphenyl)- [3,3′- bipyridine]-6- carboxamide |
| 121 | KL186- 138 | 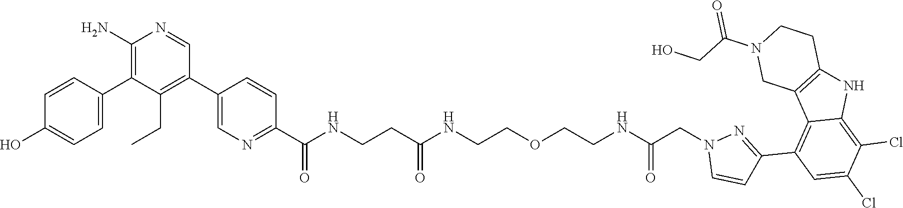 [SMILES: CCc1c(-c2ccc(C(=O)NCCC(=O)NCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((2-(2-(2-(3- (6,7-dichloro- 2-(2- hydroxyacetyl)- 2,3,4,5- tetrahydro-1H- pyrido[4,3- b]indol-9-yl)- 1H-pyrazol-1- yl)acetamido) ethoxy)- ethyl)amino)-3- oxopropyl)-4′- ethyl-5′-(4- hydroxyphenyl)- [3,3′- bipyridine]-6- carboxamide |
| 122 | KL186- 139 | 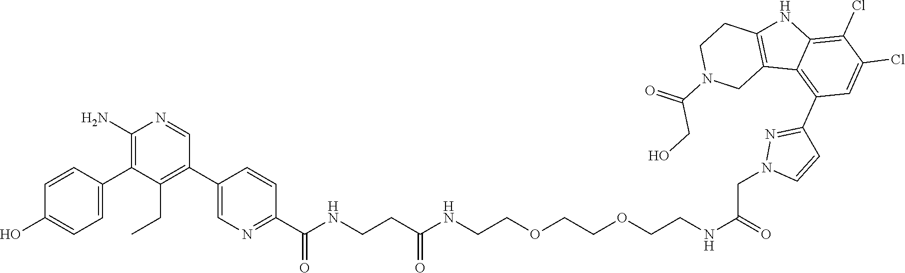 [SMILES: CCc1c(-c2ccc(C(=O)NCCC(=O)NCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(1- (3-(6,7-dichloro- 2-(2- hydroxyacetyl)- 2,3,4,5- tetrahydro-1H- pyrido[4,3- b]indol-9-yl)-1H- pyrazol-1-yl)- 2,13-dioxo-6,9- dioxa-3,12- diazapentadecan- 15-yl)-4′-ethyl- 5′-(4- hydroxyphenyl)- [3,3′- bipyridine]-6- carboxamide |
| 123 | KL186- 140 | 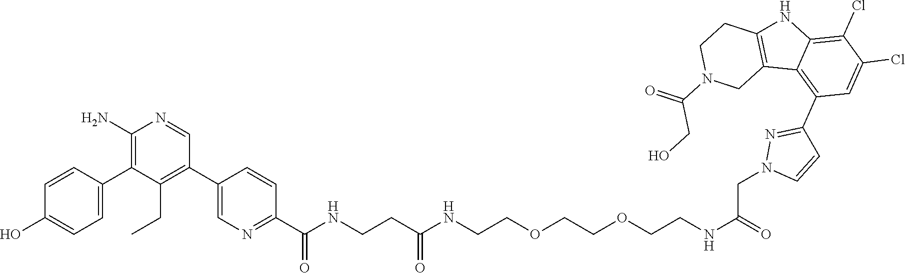 [SMILES: CCc1c(-c2ccc(C(=O)NCCC(=O)NCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(1- (3-(6,7-dichloro- 2-(2- hydroxyacetyl)- 2,3,4,5- tetrahydro-1H- pyrido[4,3- b]indol-9-yl)-1H- pyrazol-1-yl)- 2,16-dioxo- 6,9,12-trioxa- 3,15- diazaoctadecan- 18-yl)-4′-ethyl- 5′-(4- hydroxyphenyl)- [3,3′- bipyridine]-6- carboxamide |
| 124 | KL186- 141 | 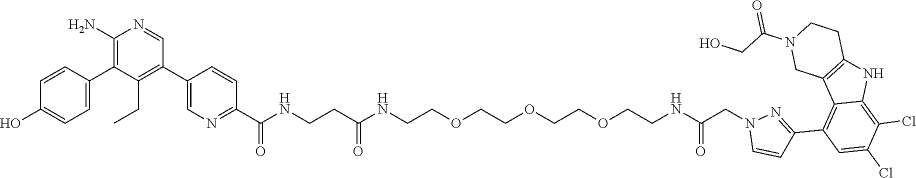 [SMILES: CCc1c(-c2ccc(C(=O)NCCC(=O)NCCOCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(1- (3-(6,7-dichloro- 2-(2- hydroxyacetyl)- 2,3,4,5- tetrahydro-1H- pyrido[4,3- b]indol-9-yl)-1H- pyrazol-1-yl)- 2,19-dioxo- 6,9,12,15- tetraoxa-3,18- diazahenicosan- 21-yl)-4′-ethyl- 5′-(4- hydroxyphenyl)- [3,3′- bipyridine]-6- carboxamide |
| 125 | KL186- 142 | 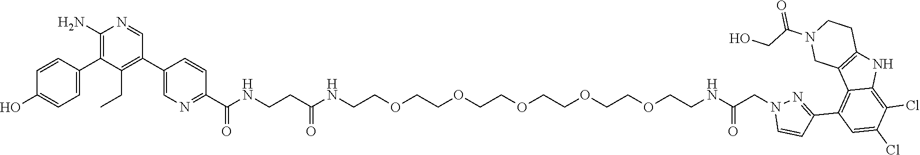 [SMILES: CCc1c(-c2ccc(C(=O)NCCC(=O)NCCOCCOCCOCCOCCOCCNC(=O)Cn3ccc(-c4cc(Cl)c(Cl)c5[nH]c6c(c45)CN(C(=O)CO)CC6)n3)nc2)cnc(N)c1-c1ccc(O)cc1] | 6′-amino-N-(1- (3-(6,7-dichloro- 2-(2- hydroxyacetyl)- 2,3,4,5- tetrahydro-1H- pyrido[4,3- b]indol-9-yl)-1H- pyrazol-1-yl)- 2,22-dioxo- 6,9,12,15,18- pentaoxa-3,21- diazatetracosan- 24-yl)-4′-ethyl- 5′-(4- hydroxyphenyl)- [3,3′- bipyridine]-6- carboxamide |
| 126 | KL186- 144 | 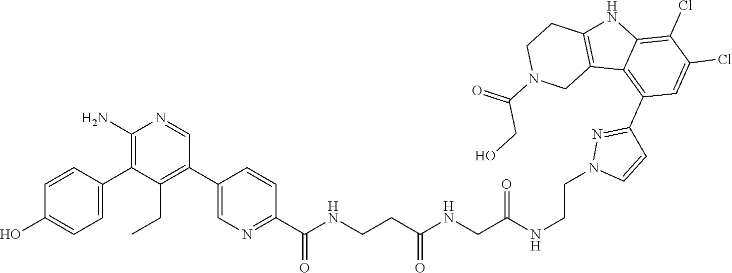 [SMILES: CCc1c(-c2ccc(C(=O)NCCC(=O)NCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((2-((2-(3-(6,7- dichloro-2-(2- hydroxyacetyl)- 2,3,4,5- tetrahydro-1H- pyrido[4,3- b]indol-9-yl)-1H- pyrazol-1- yl)ethyl)amino)- 2- oxoethyl)amino)- 3-oxopropyl)- 4′-ethyl-5′-(4- hydroxyphenyl)- [3,3′- bipyridine]-6- carboxamide |
| 127 | KL186- 145 | 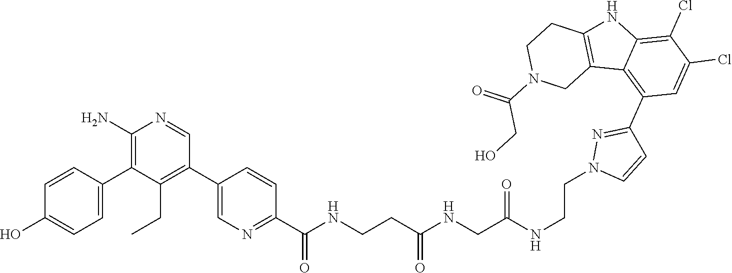 [SMILES: CCc1c(-c2ccc(C(=O)NCCC(=O)NCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((3-((2-(3-(6,7- dichloro-2-(2- hydroxyacetyl)- 2,3,4,5- tetrahydro-1H- pyrido[4,3- b]indol-9-yl)-1H- pyrazol-1- yl)ethyl)amino)- 3- oxopropyl)amino)- 3-oxopropyl)- 4′-ethyl-5′-(4- hydroxyphenyl)- [3,3′- bipyridine]-6- carboxamide |
| 128 | KL186- 146 | 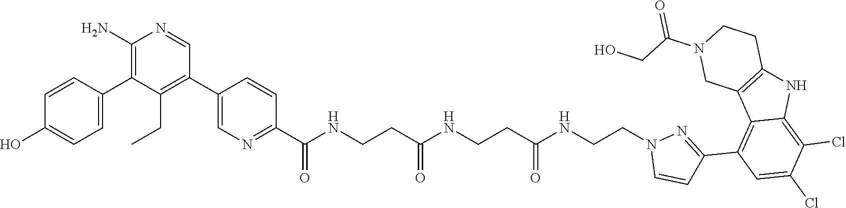 [SMILES: CCc1c(-c2ccc(C(=O)NCCC(=O)NCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((4-((2-(3-(6,7- dichloro-2-(2- hydroxyacetyl)- 2,3,4,5- tetrahydro-1H- pyrido[4,3- b]indol-9-yl)-1H- pyrazol-1- yl)ethyl)amino)- 4- oxobutyl)amino)- 3-oxopropyl)- 4′-ethyl-5′-(4- hydroxyphenyl)- [3,3′- bipyridine]-6- carboxamide |
| 129 | KL186- 147 | 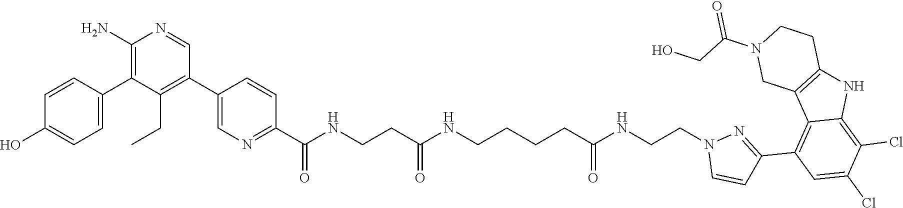 [SMILES: CCc1c(-c2ccc(C(=O)NCCC(=O)NCCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((5-((2-(3-(6,7- dichloro-2-(2- hydroxyacetyl)- 2,3,4,5- tetrahydro-1H- pyrido[4,3- b]indol-9-yl)-1H- pyrazol-1- yl)ethyl)amino)- 5- oxopentyl)amino)- 3-oxopropyl)- 4′-ethyl-5′-(4- hydroxyphenyl)- [3,3′- bipyridine]-6- carboxamide |
| 130 | KL186- 148 | 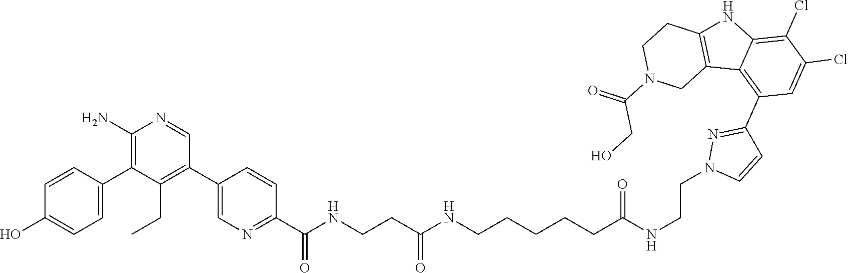 [SMILES: CCc1c(-c2ccc(C(=O)NCCC(=O)NCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((6-((2-(3-(6,7- dichloro-2-(2- hydroxyacetyl)- 2,3,4,5- tetrahydro-1H- pyrido[4,3- b]indol-9-yl)-1H- pyrazol-1- yl)ethyl)amino)- 6- oxohexyl)amino)- 3-oxopropyl)- 4′-ethyl-5′-(4- hydroxyphenyl)- [3,3′- bipyridine]-6- carboxamide |
| 131 | KL186- 149 | 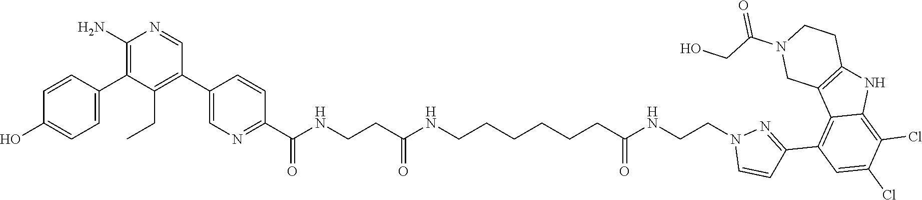 [SMILES: CCc1c(-c2ccc(C(=O)NCCC(=O)NCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((7-((2-(3-(6,7- dichloro-2-(2- hydroxyacetyl)- 2,3,4,5- tetrahydro-1H- pyrido[4,3- b]indol-9-yl)-1H- pyrazol-1- yl)ethyl)amino)- 7- oxoheptyl)amino)- 3-oxopropyl)- 4′-ethyl-5′-(4- hydroxyphenyl)- [3,3′- bipyridine]-6- carboxamide |
| 132 | KL186- 150 | 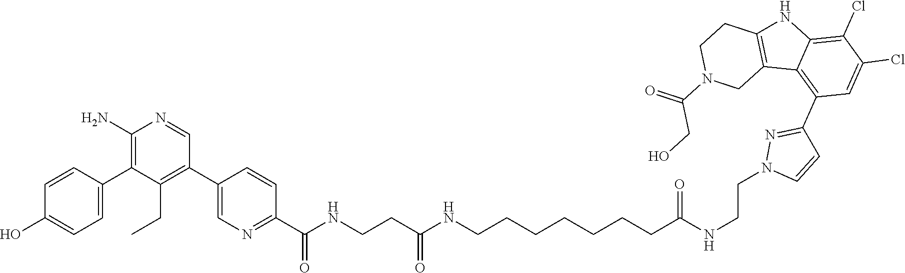 [SMILES: CCc1c(-c2ccc(C(=O)NCCC(=O)NCCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((8-((2-(3-(6,7- dichloro-2-(2- hydroxyacetyl)- 2,3,4,5- tetrahydro-1H- pyrido[4,3- b]indol-9-yl)-1H- pyrazol-1- yl)ethyl)amino)- 8- oxooctyl)amino)- 3-oxopropyl)-4′- ethyl-5′-(4- hydroxyphenyl)- [3,3′- bipyridine]-6- carboxamide |
| 133 | KL186- 151 | 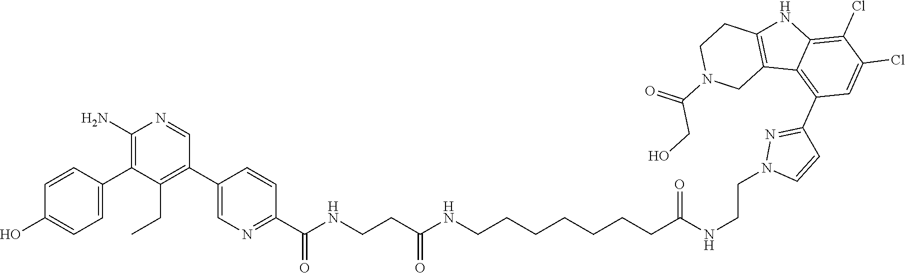 [SMILES: CCc1c(-c2ccc(C(=O)NCCC(=O)NCCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((9-((2-(3-(6,7- dichloro-2-(2- hydroxyacetyl)- 2,3,4,5- tetrahydro-1H- pyrido[4,3- b]indol-9-yl)-1H- pyrazol-1- yl)ethyl)amino)- 9- oxononyl)amino)- 3-oxopropyl)- 4′-ethyl-5′-(4- hydroxyphenyl)- [3,3′- bipyridine]-6- carboxamide |
| 134 | KL186- 152 | 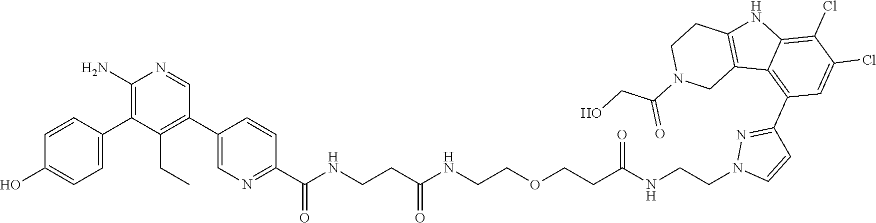 [SMILES: CCc1c(-c2ccc(C(=O)NCCC(=O)NCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(3- ((2-(3-((2-(3- (6,7-dichloro-2- (2- hydroxyacetyl)- 2,3,4,5- tetrahydro-1H- pyrido[4,3- b]indol-9-yl)-1H- pyrazol-1- yl)ethyl)amino)- 3- oxopropoxy)ethyl) amino)-3- oxopropyl)-4′- ethyl-5′-(4- hydroxyphenyl)- [3,3′- bipyridine]-6- carboxamide |
| 135 | KL186- 153 | 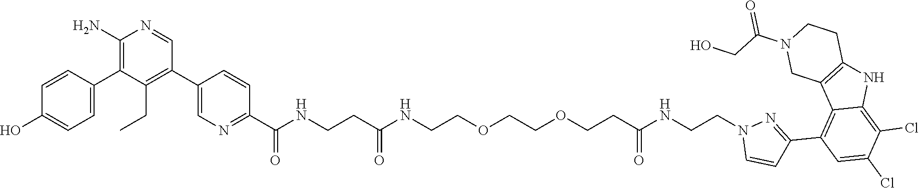 [SMILES: CCc1c(-c2ccc(C(=O)NCCC(=O)NCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(1- (3-(6,7-dichloro- 2-(2- hydroxyacetyl)- 2,3,4,5- tetrahydro-1H- pyrido[4,3- b]indol-9-yl)-1H- pyrazol-1-yl)- 4,14-dioxo-7,10- dioxa-3,13- diazahexadecan- 16-yl)-4′-ethyl- 5′-(4- hydroxyphenyl)- [3,3′- bipyridine]-6- carboxamide |
| 136 | KL186- 154 | 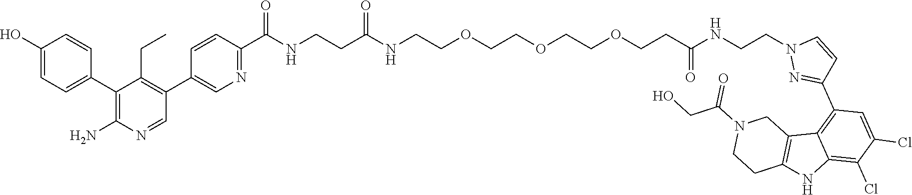 [SMILES: CCc1c(-c2ccc(C(=O)NCCC(=O)NCCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(1- (3-(6,7-dichloro- 2-(2- hydroxyacetyl)- 2,3,4,5- tetrahydro-1H- pyrido[4,3- b]indol-9-yl)-1H- pyrazol-1-yl)- 4,17-dioxo- 7,10,13-trioxa- 3,16- diazanonadecan- 19-yl)-4′-ethyl- 5′-(4- hydroxyphenyl)- [3,3′- bipyridine]-6- carboxamide |
| 137 | KL186- 155 | 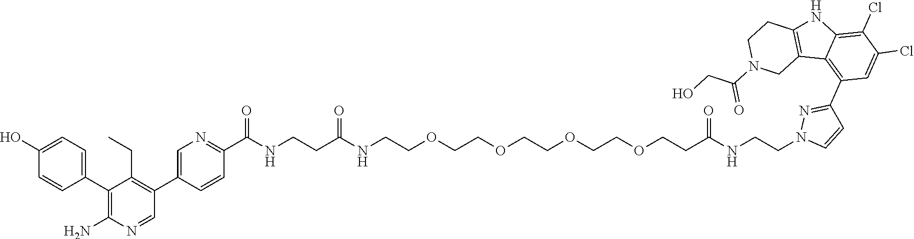 [SMILES: CCc1c(-c2ccc(C(=O)NCCC(=O)NCCOCCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(1- (3-(6,7-dichloro- 2-(2- hydroxyacetyl)- 2,3,4,5- tetrahydro-1H- pyrido[4,3- b]indol-9-yl)-1H- pyrazol-1-yl)- 4,20-dioxo- 7,10,13,16- tetraoxa-3,19- diazadocosan- 22-yl)-4′-ethyl- 5′-(4- hydroxyphenyl)- [3,3′- bipyridine]-6- carboxamide |
| 138 | KL186- 156 | 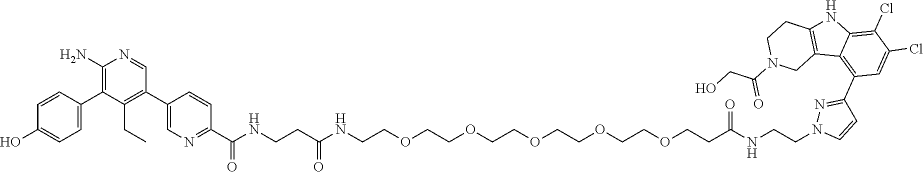 [SMILES: CCc1c(-c2ccc(C(=O)NCCC(=O)NCCOCCOCCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(1- (3-(6,7-dichloro- 2-(2- hydroxyacetyl)- 2,3,4,5- tetrahydro-1H- pyrido[4,3- b]indol-9-yl)-1H- pyrazol-1-yl)- 4,23-dioxo- 7,10,13,16,19- pentaoxa-3,22- diazapentacosan- 25-yl)-4′-ethyl- 5′-(4- hydroxyphenyl)- [3,3′- bipyridine]-6- carboxamide |
| 139 | KL186- 158 | 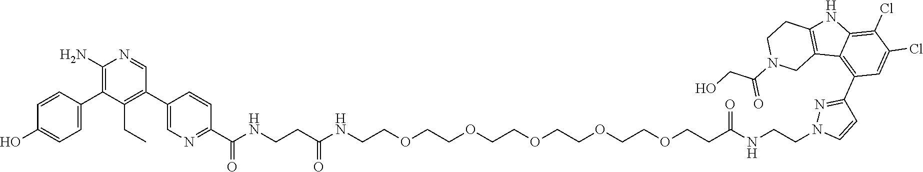 [SMILES: CCc1c(-c2ccc(C(=O)NCCC(=O)NCCOCCOCCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1] | N¹-(2-(6′-amino- 4′-ethyl-5′-(4- hydroxyphenyl)- [3,3′- bipyridine]-6- carboxamido) ethyl)-N⁴-(2-(3- (6,7-dichloro-2- (2- hydroxyacetyl)- 2,3,4,5- tetrahydro-1H- pyrido[4,3- b]indol-9-yl)-1H- pyrazol-1- yl)ethyl) succinamide |
| 140 | KL186- 159 | 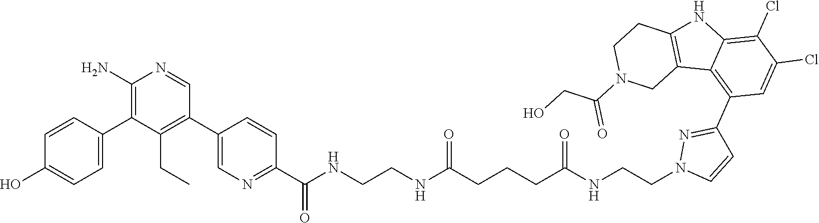 [SMILES: CCc1c(-c2ccc(C(=O)NCCNC(=O)CCCC(=O)NCCn3ccc(-c4cc(Cl)c(Cl)c5[nH]c6c(c45)CN(C(=O)CO)CC6)n3)nc2)cnc(N)c1-c1ccc(O)cc1] | N¹-(2-(6′-amino- 4′-ethyl-5′-(4- hydroxyphenyl)- [3,3′- bipyridine]-6- carboxamido) ethyl)-N⁵-(2-(3- (6,7-dichloro-2- (2- hydroxyacetyl)- 2,3,4,5- tetrahydro-1H- pyrido[4,3- b]indol-9-yl)- 1H-pyrazol-1- yl)ethyl) glutaramide |
| 141 | KL186- 160 | 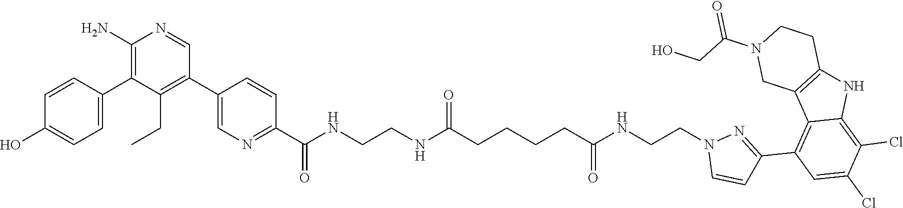 [SMILES: CCc1c(-c2ccc(C(=O)NCCNC(=O)CCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | N¹-(2-(6′-amino- 4′-ethyl-5′-(4- hydroxyphenyl)- [3,3′- bipyridine]-6- carboxamido) ethyl)-N⁶-(2-(3- (6,7-dichloro-2- (2- hydroxyacetyl)- 2,3,4,5- tetrahydro-1H- pyrido[4,3- b]indol-9-yl)-1H- pyrazol-1- yl)ethyl) adipamide |
| 142 | KL186- 161 | 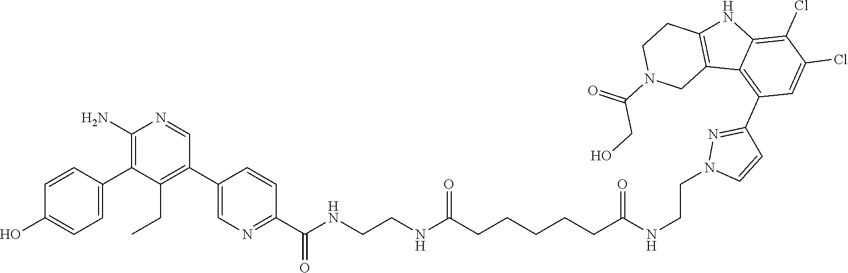 [SMILES: CCc1c(-c2ccc(C(=O)NCCNC(=O)CCCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | N¹-(2-(6′-amino- 4′-ethyl-5′-(4- hydroxyphenyl)- [3,3′- bipyridine]-6- carboxamido) ethyl)-N⁷-(2-(3- (6,7-dichloro-2- (2- hydroxyacetyl)- 2,3,4,5- tetrahydro-1H- pyrido[4,3- b]indol-9-yl)-1H- pyrazol-1- yl)ethyl) heptanediamide |
| 143 | KL186- 162 | 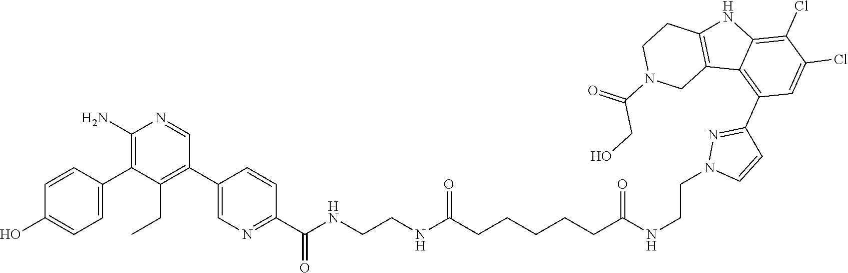 [SMILES: CCc1c(-c2ccc(C(=O)NCCNC(=O)CCCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | N¹-(2-(6′-amino- 4′-ethyl-5′-(4- hydroxyphenyl)- [3,3′- bipyridine]-6- carboxamido) ethyl)-N⁸-(2-(3- (6,7-dichloro-2- (2- hydroxyacetyl)- 2,3,4,5- tetrahydro-1H- pyrido[4,3- b]indol-9-yl)-1H- pyrazol-1- yl)ethyl) octanediamide |
| 144 | KL186- 163 | 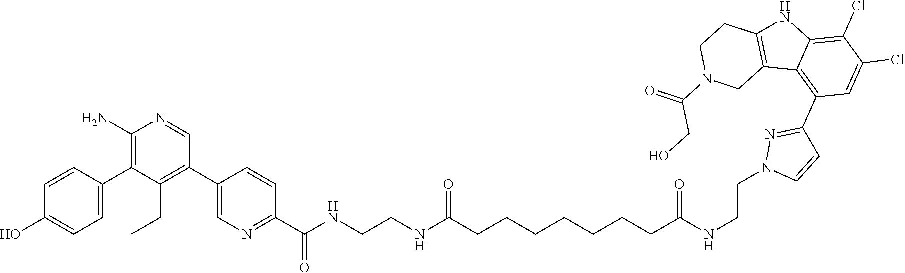 [SMILES: CCc1c(-c2ccc(C(=O)NCCNC(=O)CCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | N¹-(2-(6′-amino- 4′-ethyl-5′-(4- hydroxyphenyl)- [3,3′- bipyridine]-6- carboxamido) ethyl)-N⁹-(2-(3- (6,7-dichloro-2- (2- hydroxyacetyl)- 2,3,4,5- tetrahydro-1H- pyrido[4,3- b]indol-9-yl)-1H- pyrazol-1- yl)ethyl) nonanediamide |
| 145 | KL186- 164 | 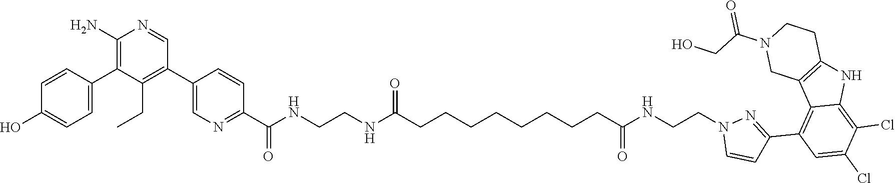 [SMILES: CCc1c(-c2ccc(C(=O)NCCNC(=O)CCCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | N¹-(2-(6′-amino- 4′-ethyl-5′-(4- hydroxyphenyl)- [3,3′- bipyridine]-6- carboxamido)et hyl)-N¹⁰-(2-(3- (6,7-dichloro-2- (2- hydroxyacetyl)- 2,3,4,5- tetrahydro-1H- pyrido[4,3- b]indol-9-yl)-1H- pyrazol-1- yl)ethyl) decanediamide |
| 146 | KL186- 165 | 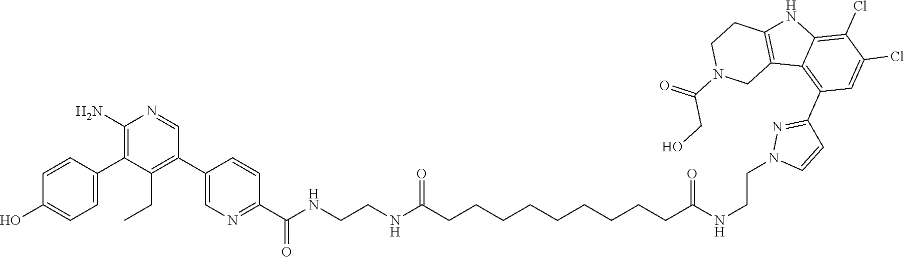 [SMILES: CCc1c(-c2ccc(C(=O)NCCNC(=O)CCCCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | N¹-(2-(6′-amino- 4′-ethyl-5′-(4- hydroxyphenyl)- [3,3′- bipyridine]-6- carboxamido) ethyl)-N¹¹-(2-(3- (6,7-dichloro-2- (2- hydroxyacetyl)- 2,3,4,5- tetrahydro-1H- pyrido[4,3- b]indol-9-yl)-1H- pyrazol-1- yl)ethyl) undecanediamide |
| 147 | KL186- 166 | 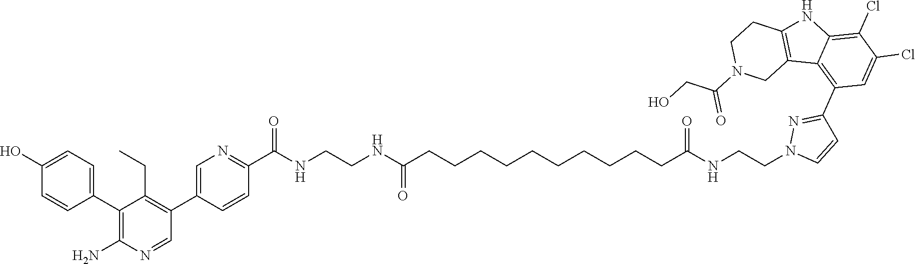 [SMILES: CCc1c(-c2ccc(C(=O)NCCNC(=O)CCCCCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | N¹-(2-(6′-amino- 4′-ethyl-5′-(4- hydroxyphenyl)- [3,3′- bipyridine]-6- carboxamido) ethyl)-N¹²-(2-(3- (6,7-dichloro-2- (2- hydroxyacetyl)- 2,3,4,5- tetrahydro-1H- pyrido[4,3- b]indol-9-yl)-1H- pyrazol-1- yl)ethyl) dodecanediamide |
| 148 | KL186- 167 | 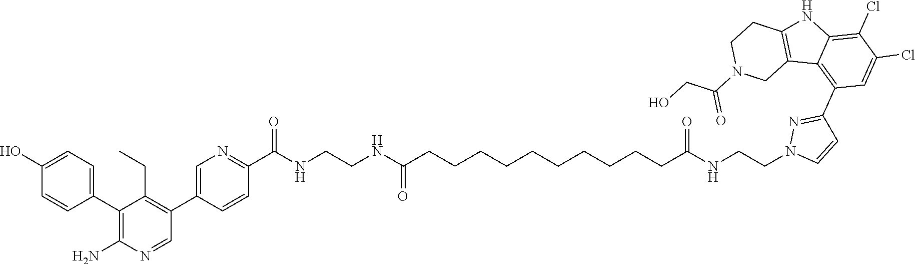 [SMILES: CCc1c(-c2ccc(C(=O)NCCNC(=O)CCCCCCCCCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(2- (3-(3-((2-(3-(6,7- dichloro-2-(2- hydroxyacetyl)- 2,3,4,5- tetrahydro-1H- pyrido[4,3- b]indol-9-yl)-1H- pyrazol-1- yl)ethyl)amino)- 3- oxopropoxy) propanamido)ethyl)- 4′-ethyl-5′-(4- hydroxyphenyl)- [3,3′- bipyridine]-6- carboxamide |
| 149 | KL186- 168 | 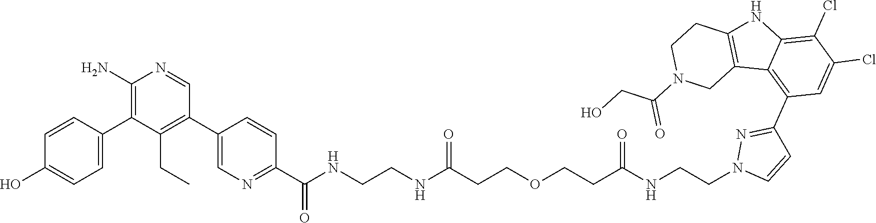 [SMILES: CCc1c(-c2ccc(C(=O)NCCNC(=O)CCOCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(16- (3-(6,7-dichloro- 2-(2- hydroxyacetyl)- 2,3,4,5- tetrahydro-1H- pyrido[4,3- b]indol-9-yl)-1H- pyrazol-1-yl)- 4,13-dioxo-7,10- dioxa-3,14- diazahexadecyl)- 4′-ethyl-5′-(4- hydroxyphenyl)- [3,3′- bipyridine]-6- carboxamide |
| 150 | KL186- 169 | 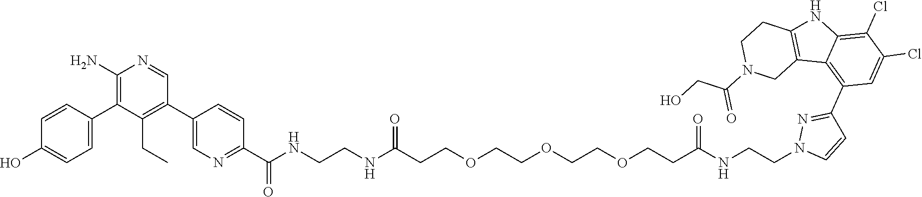 [SMILES: CCc1c(-c2ccc(C(=O)NCCNC(=O)CCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1] | 6′-amino-N-(19- (3-(6,7-dichloro- 2-(2- hydroxyacetyl)- 2,3,4,5- tetrahydro-1H- pyrido[4,3- b]indol-9-yl)-1H- pyrazol-1-yl)- 4,16-dioxo- 7,10,13-trioxa- 3,17- diazanonadecyl)- 4′-ethyl-5′-(4- hydroxyphenyl)- [3,3′- bipyridine]-6- carboxamide |
| 151 | KL186- 170 | 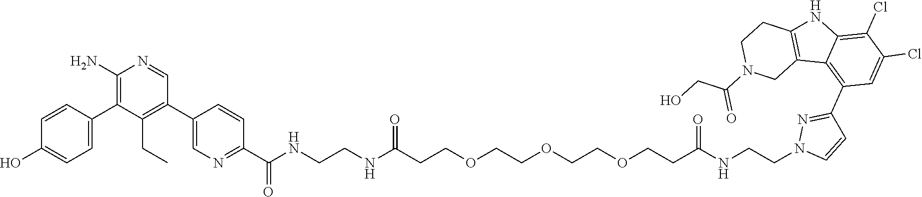 [SMILES: CCc1c(-c2ccc(C(=O)NCCNC(=O)CCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1] | ¹-(2-(6′-amino- 4′-ethyl-5′-(4- hydroxyphenyl)- [3,3′- bipyridine]-6- carboxamido) ethyl)-N¹⁶-(2-(3- (6,7-dichloro-2- (2- hydroxyacetyl)- 2,3,4,5- tetrahydro-1H- pyrido[4,3- b]indol-9-yl)-1H- pyrazol-1- yl)ethyl)- 4,7,10,13- tetraoxahexa- decanediamide |
| 152 | KL186- 171 | 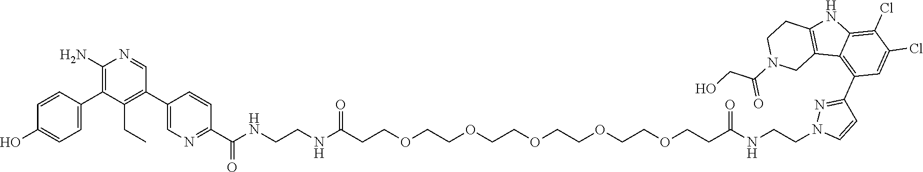 [SMILES: CCc1c(-c2ccc(C(=O)NCCNC(=O)CCOCCOCCOCCOCCOCCC(=O)NCCn3ccc(-c4cc(Cl)c(Cl)c5[nH]c6c(c45)CN(C(=O)CO)CC6)n3)nc2)cnc(N)c1-c1ccc(O)cc1] | N¹-(2-(6′-amino- 4′-ethyl-5′-(4- hydroxyphenyl)- [3,3′- bipyridine]-6- carboxamido) ethyl)-N¹⁹-(2-(3- (6,7-dichloro-2- (2- hydroxyacetyl)- 2,3,4,5- tetrahydro-1H- pyrido[4,3- b]indol-9-yl)-1H- pyrazol-1- yl)ethyl)- 4,7,10,13,16- pentaoxanonade canediamide |

Example CFTR based DUBTACs are set forth in Table 3 below.

TABLE 3

| Ex- | | | |
|---|---|---|---|
| ample | | | |
| No. | Cpd ID | Structure | name |
| 153 | XH168-51 | 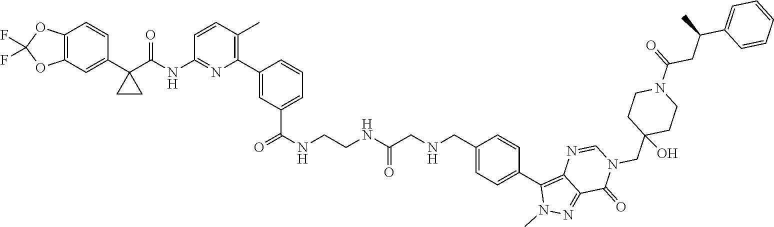 [SMILES: Cc1ccc(NC(=O)C2(c3ccc4c(c3)OC(F)(F)O4)CC2)nc1-c1cccc(C(=O)NCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(2-(2-((4- (6-((4-hydroxy- 1-(3- phenylbutanoyl) piperidin-4- yl)methyl)-2- methyl-7-oxo- |
| | | | 6,7-dihydro-2H- |
| | | | pyrazolo[4,3- |
| | | | d]pyrimidin-3- |
| | | | yl)benzyl)amino) |
| | | | acetamido)ethyl) |
| | | | benzamide |
| 154 | XH168-32 | 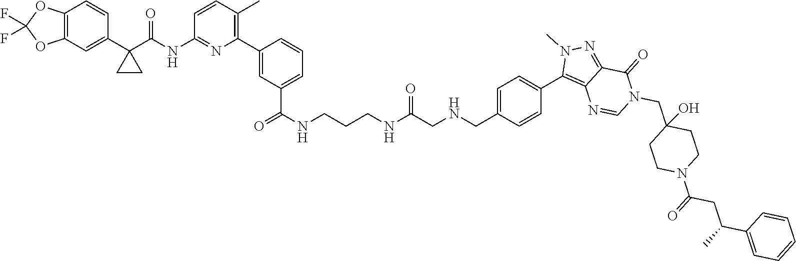 [SMILES: Cc1ccc(NC(=O)C2(c3ccc4c(c3)OC(F)(F)O4)CC2)nc1-c1cccc(C(=O)NCCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(3-(2-((4- (6-((4-hydroxy- 1-(3- phenylbutanoyl) piperidin-4- yl)methyl)-2- methyl-7-oxo- 6,7-dihydro-2H- pyrazolo[4,3- |
| | | | d]pyrimidin-3- |
| | | | yl)benzyl)amino) |
| | | | acetamido) |
| | | | propyl)benzamide |
| 155 | XH168-33 | 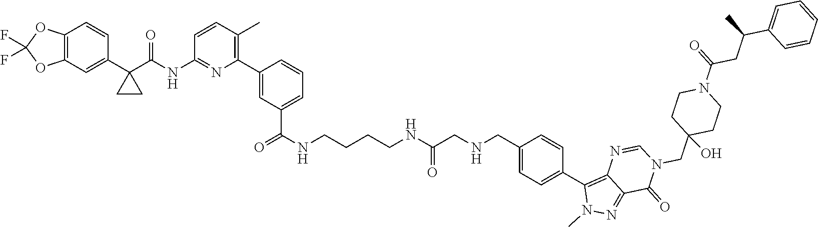 [SMILES: Cc1ccc(NC(=O)C2(c3ccc4c(c3)OC(F)(F)O4)CC2)nc1-c1cccc(C(=O)NCCCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(4-(2-((4- (6-((4-hydroxy- 1-(3- phenylbutanoyl) piperidin-4- yl)methyl)-2- methyl-7-oxo- |
| | | | 6,7-dihydro-2H- |
| | | | pyrazolo[4,3- |
| | | | d]pyrimidin-3- |
| | | | yl)benzyl)amino) |
| | | | acetamido)butyl) |
| | | | benzamide |
| 156 | XH168-59 | 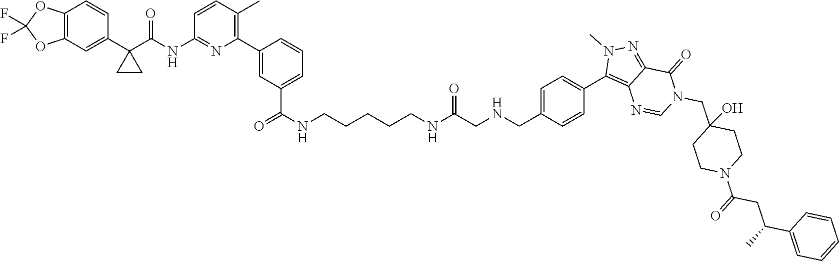 [SMILES: Cc1ccc(NC(=O)C2(c3ccc4c(c3)OC(F)(F)O4)CC2)nc1-c1cccc(C(=O)NCCCCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(5-(2-((4- (6-((4-hydroxy- 1-(3- phenylbutanoyl) piperidin-4- yl)methyl)-2- methyl-7-oxo- 6,7-dihydro-2H- pyrazolo[4,3- |
| | | | d]pyrimidin-3- |
| | | | yl)benzyl)amino) |
| | | | acetamido)pentyl) |
| | | | benzamide |
| 157 | XH168-66 | 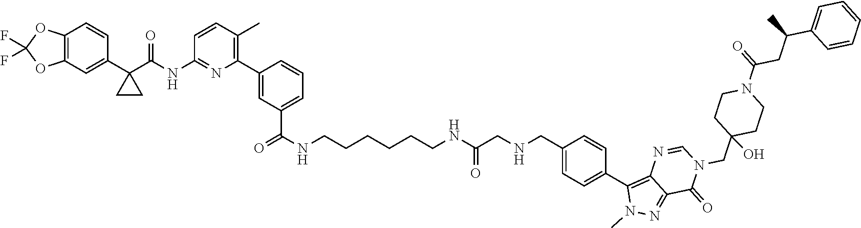 [SMILES: Cc1ccc(NC(=O)C2(c3ccc4c(c3)OC(F)(F)O4)CC2)nc1-c1cccc(C(=O)NCCCCCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(5-(2-((4- (6-((4-hydroxy- 1-(3- phenylbutanoyl) piperidin-4- yl)methyl)-2- methyl-7-oxo- |
| | | | 6,7-dihydro-2H- |
| | | | pyrazolo[4,3- |
| | | | d]pyrimidin-3- |
| | | | yl)benzyl)amino) |
| | | | acetamido)pentyl) |
| | | | benzamide |
| 158 | XH168-67 | 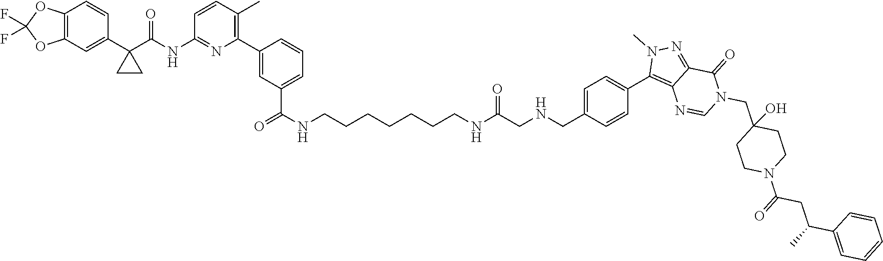 [SMILES: Cc1ccc(NC(=O)C2(c3ccc4c(c3)OC(F)(F)O4)CC2)nc1-c1cccc(C(=O)NCCCCCCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(7-(2-((4- (6-((4-hydroxy- 1-(3- phenylbutanoyl) piperidin-4- yl)methyl)-2- methyl-7-oxo- 6,7-dihydro-2H- pyrazolo[4,3- |
| | | | d]pyrimidin-3- |
| | | | yl)benzyl)amino) |
| | | | acetamido)heptyl) |
| | | | benzamide |
| 159 | XH168-68 | 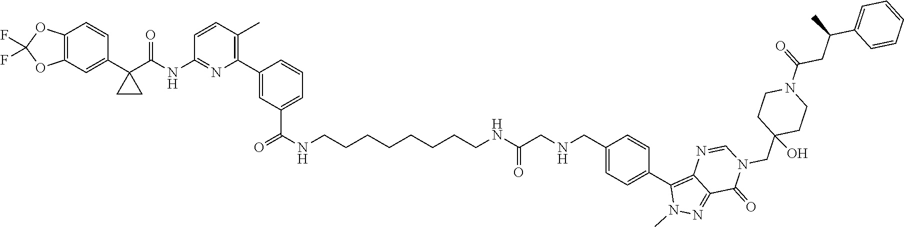 [SMILES: Cc1ccc(NC(=O)C2(c3ccc4c(c3)OC(F)(F)O4)CC2)nc1-c1cccc(C(=O)NCCCCCCCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(8-(2-((4- (6-((4-hydroxy- 1-(3- phenylbutanoyl) piperidin-4- yl)methyl)-2- methyl-7-oxo- |
| | | | 6,7-dihydro-2H- |
| | | | pyrazolo[4,3- |
| | | | d]pyrimidin-3- |
| | | | yl)benzyl)amino) |
| | | | acetamido)octyl) |
| | | | benzamide |
| 160 | XH168-69 (MS6869) | 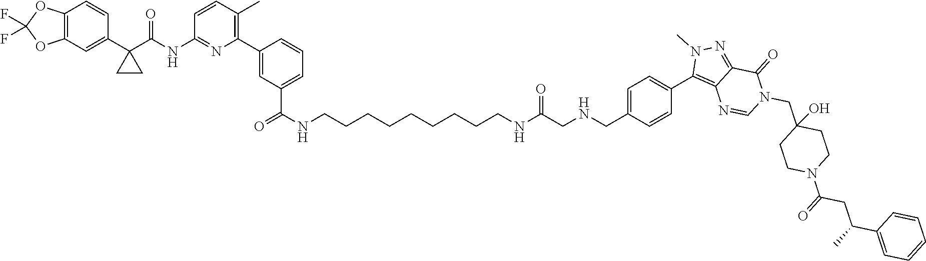 [SMILES: Cc1ccc(NC(=O)C2(c3ccc4c(c3)OC(F)(F)O4)CC2)nc1-c1cccc(C(=O)NCCCCCCCCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(9-(2-((4- (6-((4-hydroxy- 1-(3- phenylbutanoyl) piperidin-4- yl)methyl)-2- methyl-7-oxo- 6,7-dihydro-2H- pyrazolo[4,3- |
| | | | d]pyrimidin-3- |
| | | | yl)benzyl)amino) |
| | | | acetamido)nonyl) |
| | | | benzamide |
| 161 | XH168-70 | 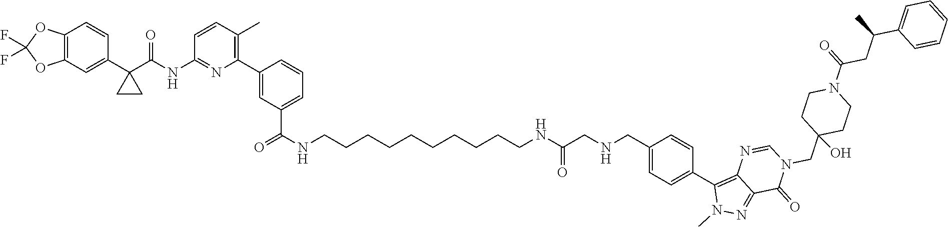 [SMILES: Cc1ccc(NC(=O)C2(c3ccc4c(c3)OC(F)(F)O4)CC2)nc1-c1cccc(C(=O)NCCCCCCCCCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(10-(2- ((4-(6-((4- hydroxy-1-(3- phenylbutanoyl) piperidin-4- yl)methyl)-2- methyl-7-oxo- |
| | | | 6,7-dihydro-2H- |
| | | | pyrazolo[4,3- |
| | | | d]pyrimidin-3- |
| | | | yl)benzyl)amino) |
| | | | acetamido)decyl) |
| | | | benzamide |
| 162 | XH168-58 | 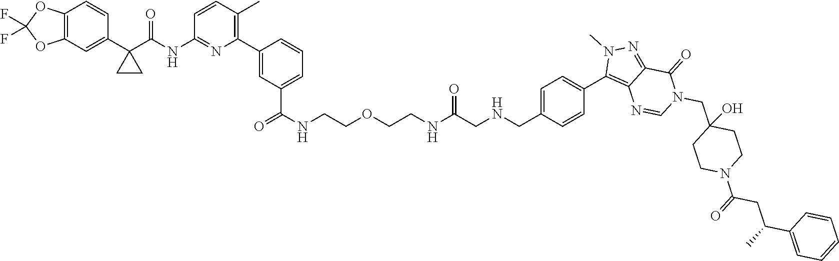 [SMILES: Cc1ccc(NC(=O)C2(c3ccc4c(c3)OC(F)(F)O4)CC2)nc1-c1cccc(C(=O)NCCOCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(2-(2-(2- ((4-(6-((4- hydroxy-1-(3- phenylbutanoyl) piperidin-4- yl)methyl)-2- methyl-7-oxo- 6,7-dihydro-2H- pyrazolo[4,3- |
| | | | d]pyrimidin-3- |
| | | | yl)benzyl)amino) |
| | | | acetamido)ethoxy) |
| | | | ethyl)benzamide |
| 163 | XH168-47 | 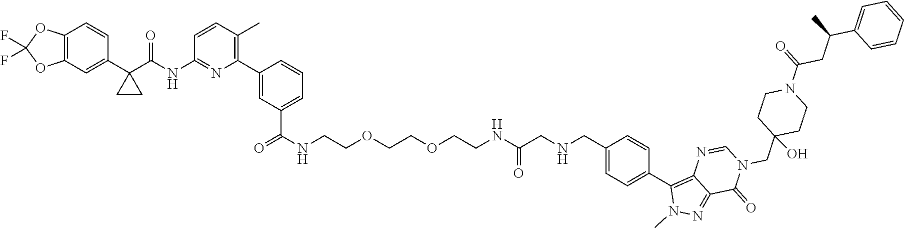 [SMILES: Cc1ccc(NC(=O)C2(c3ccc4c(c3)OC(F)(F)O4)CC2)nc1-c1cccc(C(=O)NCCOCCOCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(1-(4-(6- ((4-hydroxy-1- (3- phenylbutanoyl) piperidin-4- yl)methyl)-2- methyl-7-oxo- |
| | | | 6,7-dihydro-2H- |
| | | | pyrazolo[4,3- |
| | | | d]pyrimidin-3- |
| | | | yl)phenyl)-4- |
| | | | oxo-8,11-dioxa- |
| | | | 2,5- |
| | | | diazatridecan- |
| | | | 13-yl)benzamide |
| 164 | XH168-48 | 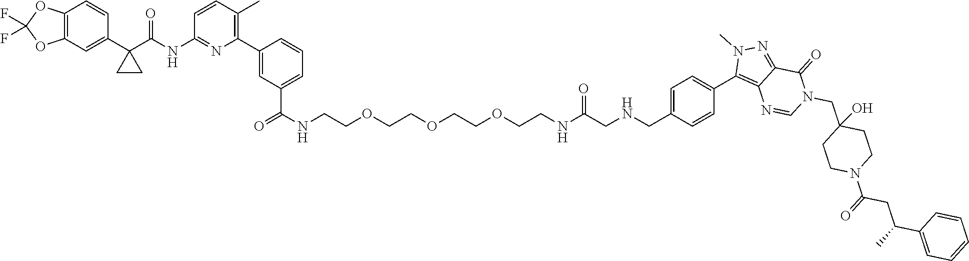 [SMILES: Cc1ccc(NC(=O)C2(c3ccc4c(c3)OC(F)(F)O4)CC2)nc1-c1cccc(C(=O)NCCOCCOCCOCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(1-(4-(6- ((4-hydroxy-1- (3- phenylbutanoyl) piperidin-4- yl)methyl)-2- methyl-7-oxo- 6,7-dihydro-2H- pyrazolo[4,3- |
| | | | d]pyrimidin-3- |
| | | | yl)phenyl)-4- |
| | | | oxo-8,11,14- |
| | | | trioxa-2,5- |
| | | | diazahexadecan- |
| | | | 16-yl)benzamide |
| 165 | XH168-49 | 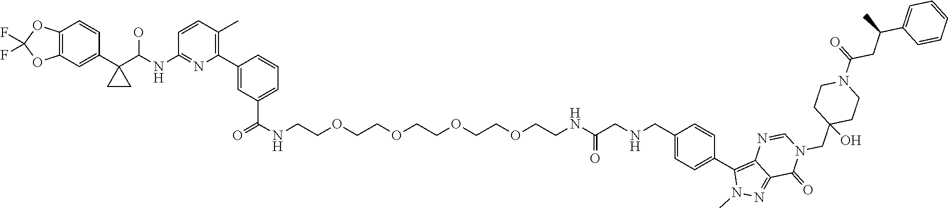 [SMILES: Cc1ccc(NC(O)C2(c3ccc4c(c3)OC(F)(F)O4)CC2)nc1-c1cccc(C(=O)NCCOCCOCCOCCOCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(1-(4-(6- ((4-hydroxy-1- (3- phenylbutanoyl) piperidin-4- yl)methyl)-2- |
| | | | methyl-7-oxo- |
| | | | 6,7-dihydro-2H- |
| | | | pyrazolo[4,3- |
| | | | d]pyrimidin-3- |
| | | | yl)phenyl)-4- |
| | | | oxo-8,11,14,17- |
| | | | tetraoxa-2,5- |
| | | | diazanonadecan- |
| | | | 19-yl)benzamide |
| 166 | XH168-50 | 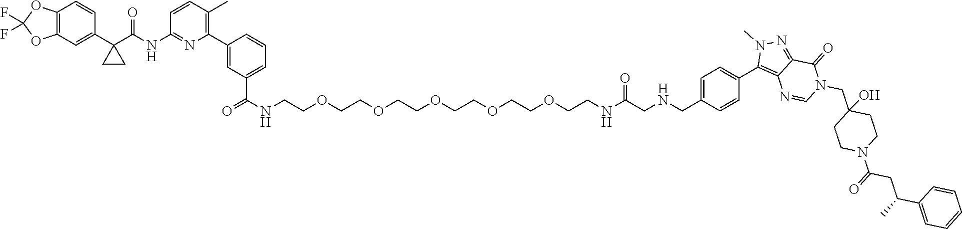 [SMILES: Cc1ccc(NC(=O)C2(c3ccc4c(c3)OC(F)(F)O4)CC2)nc1-c1cccc(C(=O)NCCOCCOCCOCCOCCOCCNC(=O)CNCc2ccc(-c3c4ncn(CC5(O)CCN(C(=O)C[C@@H](C)c6ccccc6)CC5)c(=O)c4nn3C)cc2)c1] | (R)-3-(6-(1-(2,2- difluorobenzo[d] [1,3]dioxol-5- yl)cyclopropane- 1-carboxamido)- 3- methylpyridin- 2-yl)-N-(1-(4-(6- ((4-hydroxy-1- (3- phenylbutanoyl) piperidin-4- yl)methyl)-2- methyl-7-oxo- 6,7-dihydro-2H- |
| | | | pyrazolo[4,3- |
| | | | d]pyrimidin-3- |
| | | | yl)phenyl)-4- |
| | | | oxo- |
| | | | 8,11,14,17,20- |
| | | | pentaoxa-2,5- |
| | | | diazadocosan- |
| | | | 22-yl)benzamide |
| 167 | KL180-124 | 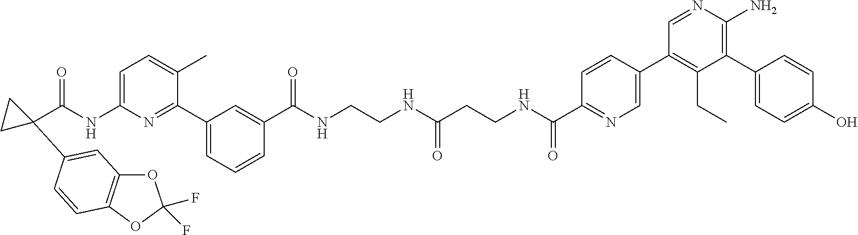 [SMILES: CCc1c(-c2ccc(C(=O)NCCC(=O)NCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(3- ((2-(3-(6-(1-(2,2- difluorobenzo[d] [1,3]dioxol-5- yl)cyclopropane- 1-carboxamido)- 3- methylpyridin- 2- yl)benzamido) ethyl)amino)-3- oxopropyl)-4′- ethyl-5′-(4- hydroxyphenyl)- [3,3′- |
| | | | bipyridine]-6- |
| | | | carboxamide |
| 168 | KL180-125 | 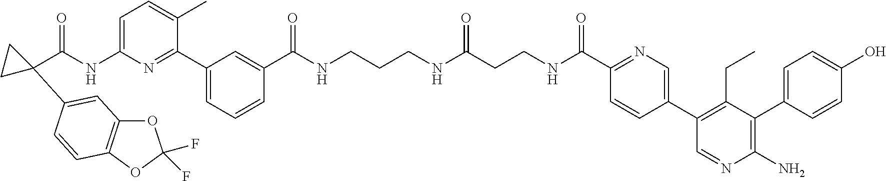 [SMILES: CCc1c(-c2ccc(C(=O)NCCC(=O)NCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(3- ((3-(3-(6-(1-(2,2- difluorobenzo[d] [1,3]dioxol-5- yl)cyclopropane- 1-carboxamido)- 3- methylpyridin- 2- yl)benzamido) propyl)amino)-3- |
| | | | oxopropyl)-4′- |
| | | | ethyl-5′-(4- |
| | | | hydroxyphenyl)- |
| | | | [3,3′- |
| | | | bipyridine]-6- |
| | | | carboxamide |
| 169 | KL180-126 | 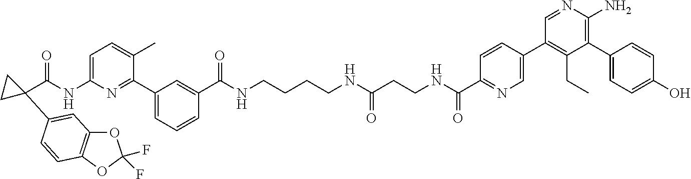 [SMILES: CCc1c(-c2ccc(C(=O)NCCC(=O)NCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(3- ((4-(3-(6-(1-(2,2- difluorobenzo[d] [1,3]dioxol-5- yl)cyclopropane- 1-carboxamido)- 3- methylpyridin- 2- yl)benzamido) butyl)amino)-3- |
| | | | oxopropyl)-4′- |
| | | | ethyl-5′-(4- |
| | | | hydroxyphenyl)- |
| | | | [3,3′- |
| | | | bipyridine]-6- |
| | | | carboxamide |
| 170 | KL180-127 | 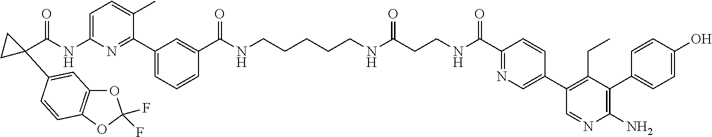 [SMILES: CCc1c(-c2ccc(C(=O)NCCC(=O)NCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(3- ((5-(3-(6-(1-(2,2- difluorobenzo[d] [1,3]dioxol-5- yl)cyclopropane- 1-carboxamido)- 3- methylpyridin- 2- |
| | | | yl)benzamido) |
| | | | pentyl)amino)-3- |
| | | | oxopropyl)-4′- |
| | | | ethyl-5′-(4- |
| | | | hydroxyphenyl)- |
| | | | [3,3′- |
| | | | bipyridine]-6- |
| | | | carboxamide |
| 171 | KL180-128 | 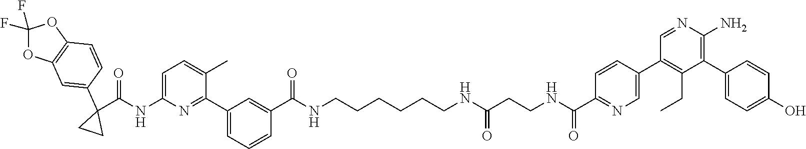 [SMILES: CCc1c(-c2ccc(C(=O)NCCC(=O)NCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(3- ((6-(3-(6-(1-(2,2- difluorobenzo[d] [1,3]dioxol-5- yl)cyclopropane- 1-carboxamido)- 3- methylpyridin- 2- yl)benzamido) |
| | | | hexyl)amino)-3- |
| | | | oxopropyl)-4′- |
| | | | ethyl-5′-(4- |
| | | | hydroxyphenyl)- |
| | | | [3,3′- |
| | | | bipyridine]-6- |
| | | | carboxamide |
| 172 | KL180-129 | 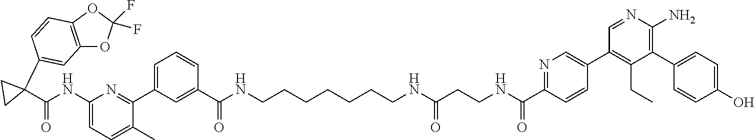 [SMILES: CCc1c(-c2ccc(C(=O)NCCC(=O)NCCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(3- ((7-(3-(6-(1-(2,2- difluorobenzo[d] [1,3]dioxol-5- yl)cyclopropane- 1-carboxamido)- 3- methylpyridin- 2- |
| | | | yl)benzamido) |
| | | | heptyl)amino)-3- |
| | | | oxopropyl)-4′- |
| | | | ethyl-5′-(4- |
| | | | hydroxyphenyl)- |
| | | | [3,3′- |
| | | | bipyridine]-6- |
| | | | carboxamide |
| 173 | KL180-130 | 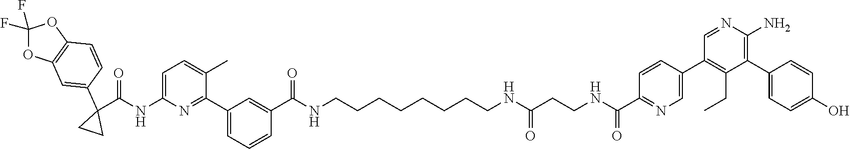 [SMILES: CCc1c(-c2ccc(C(=O)NCCC(=O)NCCCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(3- ((8-(3-(6-(1-(2,2- difluorobenzo[d] [1,3]dioxol-5- yl)cyclopropane- 1-carboxamido)- 3- methylpyridin- 2- yl)benzamido) |
| | | | octyl)amino)-3- |
| | | | oxopropyl)-4′- |
| | | | ethyl-5′-(4- |
| | | | hydroxyphenyl)- |
| | | | [3,3′- |
| | | | bipyridine]-6- |
| | | | carboxamide |
| 174 | KL180-131 | 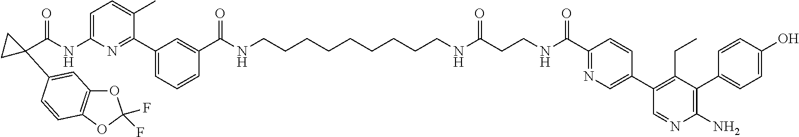 [SMILES: CCc1c(-c2ccc(C(=O)NCCC(=O)NCCCCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(3- ((9-(3-(6-(1-(2,2- difluorobenzo[d] [1,3]dioxol-5- yl)cyclopropane- 1-carboxamido)- 3- methylpyridin- 2- |
| | | | yl)benzamido) |
| | | | nonyl)amino)-3- |
| | | | oxopropyl)-4′- |
| | | | ethyl-5′-(4- |
| | | | hydroxyphenyl)- |
| | | | [3,3′- |
| | | | bipyridine]-6- |
| | | | carboxamide |
| 175 | KL180-132 | 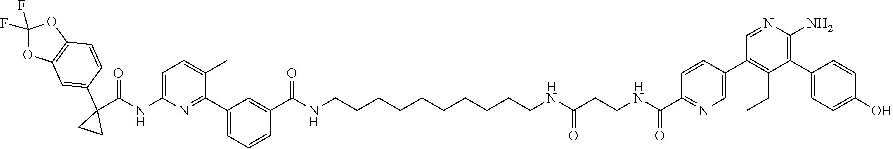 [SMILES: CCc1c(-c2ccc(C(=O)NCCC(=O)NCCCCCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(3- ((10-(3-(6-(1- (2,2- difluorobenzo[d] [1,3]dioxol-5- yl)cyclopropane- 1-carboxamido)- 3- methylpyridin- 2- |
| | | | yl)benzamido) |
| | | | decyl)amino)-3- |
| | | | oxopropyl)-4′- |
| | | | ethyl-5′-(4- |
| | | | hydroxyphenyl)- |
| | | | [3,3′- |
| | | | bipyridine]-6- |
| | | | carboxamide |
| 176 | KL180-133 | 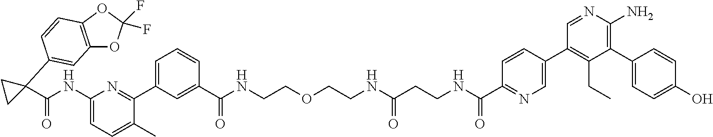 [SMILES: CCc1c(-c2ccc(C(=O)NCCC(=O)NCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(3- ((2-(2-(3-(6-(1- (2,2- difluorobenzo[d] [1,3]dioxol-5- yl)cyclopropane- 1-carboxamido)- 3- methylpyridin- |
| | | | 2- |
| | | | yl)benzamido) |
| | | | ethoxy)ethyl) |
| | | | amino)-3- |
| | | | oxopropyl)- |
| | | | 4′-ethyl-5′-(4- |
| | | | hydroxyphenyl)- |
| | | | [3,3′- |
| | | | bipyridine]-6- |
| | | | carboxamide |
| 177 | KL180-134 | 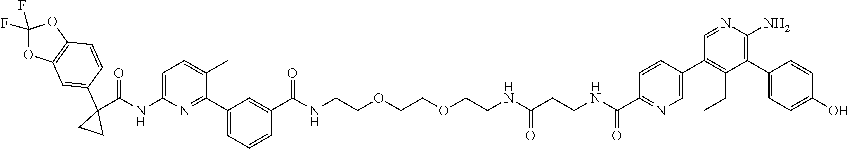 [SMILES: CCc1c(-c2ccc(C(=O)NCCC(=O)NCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(1- (3-(6-(1-(2,2- difluorobenzo[d] [1,3]dioxol-5- yl)cyclopropane- 1-carboxamido)- 3- methylpyridin- 2-yl)phenyl)- 1,12-dioxo-5,8- |
| | | | dioxa-2,11- |
| | | | diazatetradecan- |
| | | | 14-yl)-4′-ethyl- |
| | | | 5′-(4- |
| | | | hydroxyphenyl)- |
| | | | [3,3′- |
| | | | bipyridine]-6- |
| | | | carboxamide |
| 178 | KL180-135 | 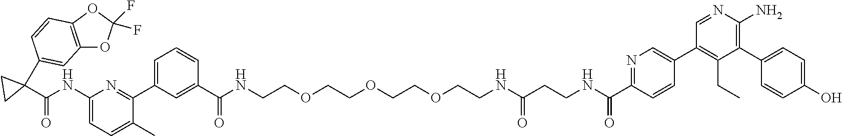 [SMILES: CCc1c(-c2ccc(C(=O)NCCC(=O)NCCOCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(1- (3-(6-(1-(2,2- difluorobenzo[d] [1,3]dioxol-5- yl)cyclopropane- 1-carboxamido)- 3- methylpyridin- 2-yl)phenyl)- |
| | | | 1,15-dioxo- |
| | | | 5,8,11-trioxa- |
| | | | 2,14- |
| | | | diazaheptadecan- |
| | | | 17-yl)-4′-ethyl- |
| | | | 5′-(4- |
| | | | hydroxyphenyl)- |
| | | | [3,3′- |
| | | | bipyridine]-6- |
| | | | carboxamide |
| 179 | KL180-136 | 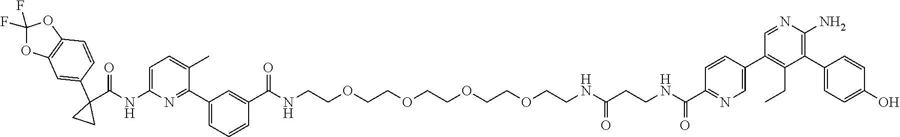 [SMILES: CCc1c(-c2ccc(C(=O)NCCC(=O)NCCOCCOCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(1- (3-(6-(1-(2,2- difluorobenzo[d] [1,3]dioxol-5- yl)cyclopropane- 1-carboxamido)- 3- methylpyridin- 2-yl)phenyl)- |
| | | | 1,18-dioxo- |
| | | | 5,8,11,14- |
| | | | tetraoxa-2,17- |
| | | | diazaicosan-20- |
| | | | yl)-4′-ethyl-5′- |
| | | | (4- |
| | | | hydroxyphenyl)- |
| | | | [3,3′- |
| | | | bipyridine]-6- |
| | | | carboxamide |
| 180 | KL180-137 | 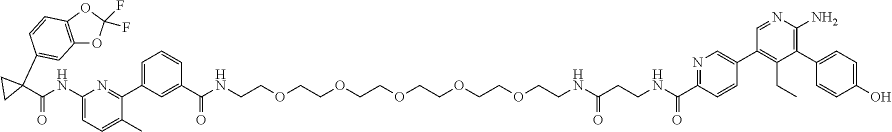 [SMILES: CCc1c(-c2ccc(C(=O)NCCC(=O)NCCOCCOCCOCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(1- (3-(6-(1-(2,2- difluorobenzo[d] [1,3]dioxol-5- yl)cyclopropane- 1-carboxamido)- 3- methylpyridin- |
| | | | 2-yl)phenyl)- |
| | | | 1,21-dioxo- |
| | | | 5,8,11,14,17- |
| | | | pentaoxa-2,20- |
| | | | diazatricosan- |
| | | | 23-yl)-4′-ethyl- |
| | | | 5′-(4- |
| | | | hydroxyphenyl)- |
| | | | [3,3′- |
| | | | bipyridine]-6- |
| | | | carboxamide |
| 181 | KL180-162 | 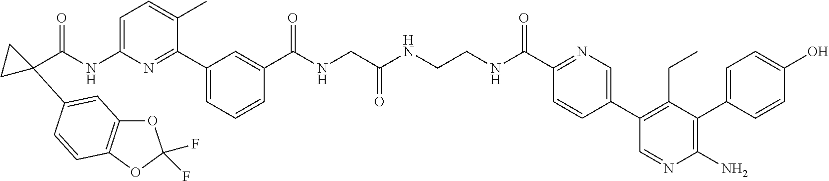 [SMILES: CCc1c(-c2ccc(C(=O)NCCNC(=O)CNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(2- (2-(3-(6-(1-(2,2- difluorobenzo[d] [1,3]dioxol-5- yl)cyclopropane- 1-carboxamido)- 3- methylpyridin- 2- yl)benzamido) acetamido)ethyl)- |
| | | | 4′-ethyl-5′-(4- |
| | | | hydroxyphenyl)- |
| | | | [3,3′- |
| | | | bipyridine]-6- |
| | | | carboxamide |
| 182 | KL180-163 | 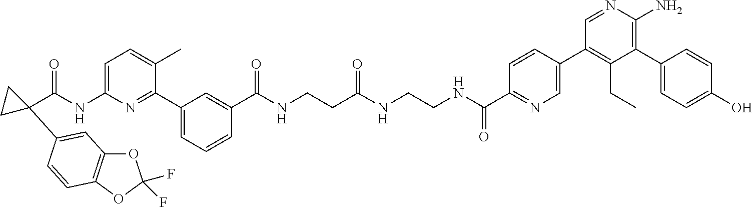 [SMILES: CCc1c(-c2ccc(C(=O)NCCNC(=O)CCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(2- (3-(3-(6-(1-(2,2- difluorobenzo[d] [1,3]dioxol-5- yl)cyclopropane- 1-carboxamido)- 3- methylpyridin- 2- yl)benzamido) propanamido) ethyl)- 4′-ethyl-5′-(4- |
| | | | hydroxyphenyl)- |
| | | | [3,3′- |
| | | | bipyridine]-6- |
| | | | carboxamide |
| 183 | KL180-164 | 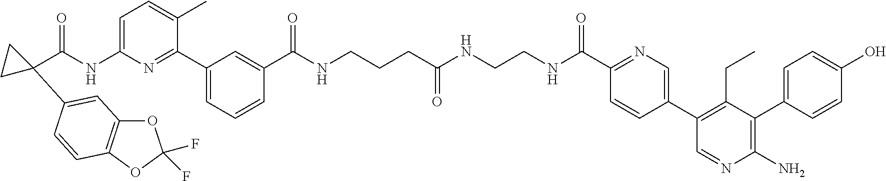 [SMILES: CCc1c(-c2ccc(C(=O)NCCNC(=O)CCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(2- (4-(3-(6-(1-(2,2- difluorobenzo[d] [1,3]dioxol-5- yl)cyclopropane- 1-carboxamido)- 3- methylpyridin- 2- yl)benzamido) butanamido) ethyl)- |
| | | | 4′-ethyl-5′-(4- |
| | | | hydroxyphenyl)- |
| | | | [3,3′- |
| | | | bipyridine]-6- |
| | | | carboxamide |
| 184 | KL180-165 | 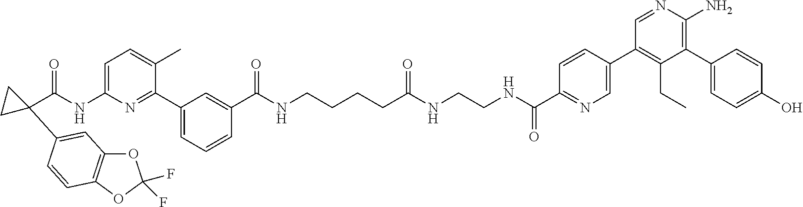 [SMILES: CCc1c(-c2ccc(C(=O)NCCNC(=O)CCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(2- (5-(3-(6-(1-(2,2- difluorobenzo[d] [1,3]dioxol-5- yl)cyclopropane- 1-carboxamido)- 3- methylpyridin- 2- yl)benzamido) pentanamido) ethyl)- 4′-ethyl-5′-(4- |
| | | | hydroxyphenyl)- |
| | | | [3,3′- |
| | | | bipyridine]-6- |
| | | | carboxamide |
| 185 | KL180-166 | 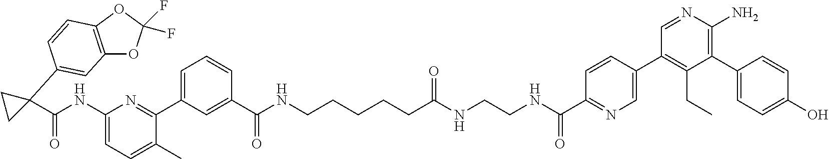 [SMILES: CCc1c(-c2ccc(C(=O)NCCNC(=O)CCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(2- (6-(3-(6-(1-(2,2- difluorobenzo[d] [1,3]dioxol-5- yl)cyclopropane- 1-carboxamido)- 3- methylpyridin- 2- yl)benzamido) |
| | | | hexanamido) |
| | | | ethyl)-4′- |
| | | | ethyl-5′-(4- |
| | | | hydroxyphenyl)- |
| | | | [3,3′- |
| | | | bipyridine]-6- |
| | | | carboxamide |
| 186 | KL180-167 | 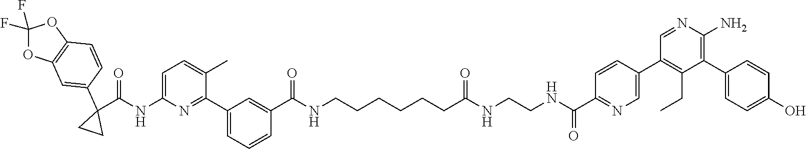 [SMILES: CCc1c(-c2ccc(C(=O)NCCNC(=O)CCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(2- (7-(3-(6-(1-(2,2- difluorobenzo[d] [1,3]dioxol-5- yl)cyclopropane- 1-carboxamido)- 3- methylpyridin- 2- yl)benzamido) |
| | | | heptanamido) |
| | | | ethyl)-4′- |
| | | | ethyl-5′-(4- |
| | | | hydroxyphenyl)- |
| | | | [3,3′- |
| | | | bipyridine]-6- |
| | | | carboxamide |
| 187 | KL180-168 | 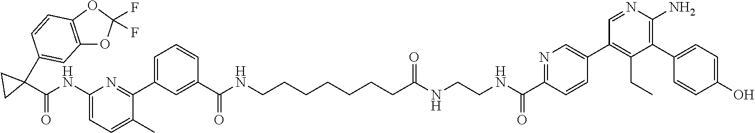 [SMILES: CCc1c(-c2ccc(C(=O)NCCNC(=O)CCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(2- (8-(3-(6-(1-(2,2- difluorobenzo[d] [1,3]dioxol-5- yl)cyclopropane- 1-carboxamido)- 3- methylpyridin- 2- |
| | | | yl)benzamido) |
| | | | octanamido)ethyl)- |
| | | | 4′-ethyl-5′-(4- |
| | | | hydroxyphenyl)- |
| | | | [3,3′- |
| | | | bipyridine]-6- |
| | | | carboxamide |
| 188 | KL180-169 | 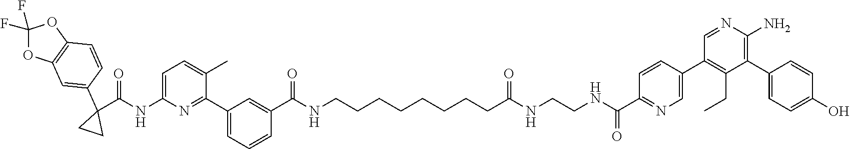 [SMILES: CCc1c(-c2ccc(C(=O)NCCNC(=O)CCCCCCCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(2- (9-(3-(6-(1-(2,2- difluorobenzo[d] [1,3]dioxol-5- yl)cyclopropane- 1-carboxamido)- 3- methylpyridin- 2- yl)benzamido) |
| | | | nonanamido)ethyl)- |
| | | | 4′-ethyl-5′-(4- |
| | | | hydroxyphenyl)- |
| | | | [3,3′- |
| | | | bipyridine]-6- |
| | | | carboxamide |
| 189 | KL180-170 | 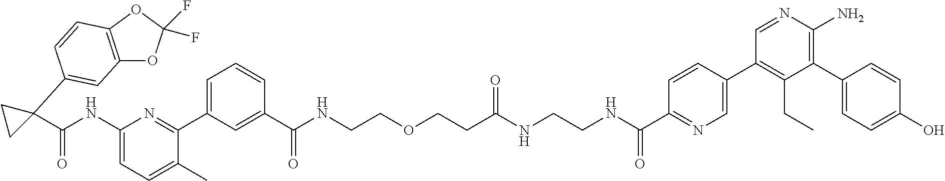 [SMILES: CCc1c(-c2ccc(C(=O)NCCNC(=O)CCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(2- (3-(2-(3-(6-(1- (2,2- difluorobenzo [d][1,3]dioxol-5- yl)cyclopropane- 1- carboxamido)- 3- methylpyridin- 2- |
| | | | yl)benzamido) |
| | | | ethoxy) |
| | | | propanamido)- |
| | | | ethyl)-4′-ethyl- |
| | | | 5′-(4- |
| | | | hydroxyphenyl)- |
| | | | [3,3′- |
| | | | bipyridine]-6- |
| | | | carboxamide |
| 190 | KL180-171 | 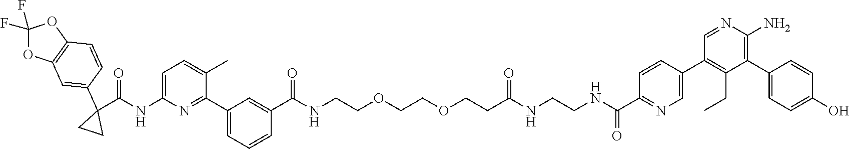 [SMILES: CCc1c(-c2ccc(C(=O)NCCNC(=O)CCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(1- (3-(6-(1-(2,2- difluorobenzo[d] [1,3]dioxol-5- yl)cyclopropane- 1-carboxamido)- 3- methylpyridin- 2-yl)phenyl)- 1,11-dioxo-5,8- |
| | | | dioxa-2,12- |
| | | | diazatetradecan- |
| | | | 14-yl)-4′-ethyl- |
| | | | 5′-(4- |
| | | | hydroxyphenyl)- |
| | | | [3,3′- |
| | | | bipyridine]-6- |
| | | | carboxamide |
| 191 | KL180-172 | 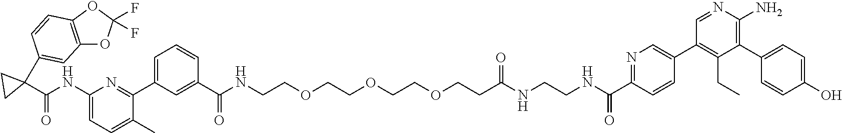 [SMILES: CCc1c(-c2ccc(C(=O)NCCNC(=O)CCOCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(1- (3-(6-(1-(2,2- difluorobenzo[d] [1,3]dioxol-5- yl)cyclopropane- 1-carboxamido)- 3- methylpyridin- 2-yl)phenyl)- |
| | | | 1,14-dioxo- |
| | | | 5,8,11-trioxa- |
| | | | 2,15- |
| | | | diazaheptadecan- |
| | | | 17-yl)-4′-ethyl- |
| | | | 5′-(4- |
| | | | hydroxyphenyl)- |
| | | | [3,3′- |
| | | | bipyridine]-6- |
| | | | carboxamide |
| 192 | KL180-173 | 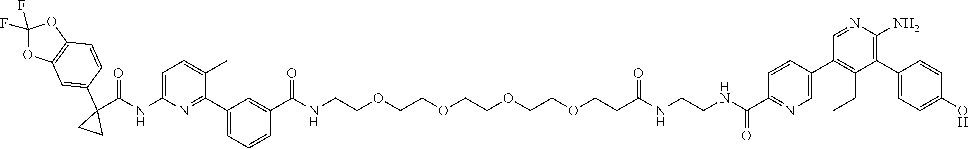 [SMILES: CCc1c(-c2ccc(C(=O)NCCNC(=O)CCOCCOCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(1- (3-(6-(1-(2,2- difluorobenzo[d] [1,3]dioxol-5- yl)cyclopropane- 1-carboxamido)- 3- methylpyridin- 2-yl)phenyl)- 1,17-dioxo- |
| | | | 5,8,11,14- |
| | | | tetraoxa-2,18- |
| | | | diazaicosan-20- |
| | | | yl)-4′-ethyl-5′- |
| | | | (4- |
| | | | hydroxyphenyl)- |
| | | | [3,3′- |
| | | | bipyridine]-6- |
| | | | carboxamide |
| 193 | KL180-174 | 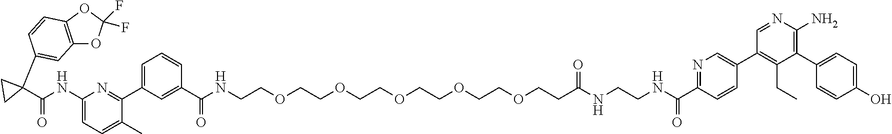 [SMILES: CCc1c(-c2ccc(C(=O)NCCNC(=O)CCOCCOCCOCCOCCOCCNC(=O)c3cccc(-c4nc(NC(=O)C5(c6ccc7c(c6)OC(F)(F)O7)CC5)ccc4C)c3)nc2)cnc(N)c1-c1ccc(O)cc1] | 6′-amino-N-(1- (3-(6-(1-(2,2- difluorobenzo[d] [1,3]dioxol-5- yl)cyclopropane- 1-carboxamido)- 3- methylpyridin- |
| | | | 2-yl)phenyl)- |
| | | | 1,20-dioxo- |
| | | | 5,8,11,14,17- |
| | | | pentaoxa-2,21- |
| | | | diazatricosan- |
| | | | 23-yl)-4′-ethyl- |
| | | | 5′-(4- |
| | | | hydroxyphenyl)- |
| | | | [3,3′- |
| | | | bipyridine]-6- |
| | | | carboxamide |

### Example 194. Assessment of the Effect of AMPK Based Bivalent Compounds (XH Series) on Stabilization of the AMPK Protein Level in Hela Cells Using WB Assay (FIG. 1)

The Hela cells was treated with DMSO, or selected bivalent compound at 10 μM for 24 h. The Western blot results showed that multiple compounds can increase AMPKB1 protein level.

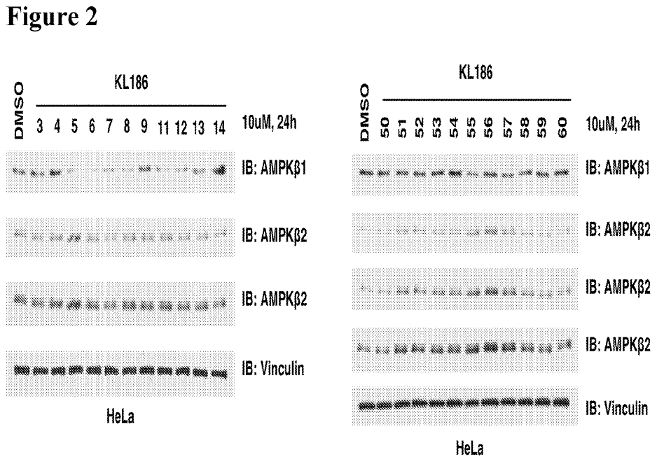

### Example 195. Assessment of the Effect of AMPK Based Bivalent Compounds (KL Series) on Stabilization of the AMPK Protein Level in Hela Cells Using WB Assay (FIG. 2)

The Hela cells was treated with DMSO, or selected bivalent compound at 10 UM for 24 h. The Western blot results showed that multiple compounds can increase AMPKB2 protein level.

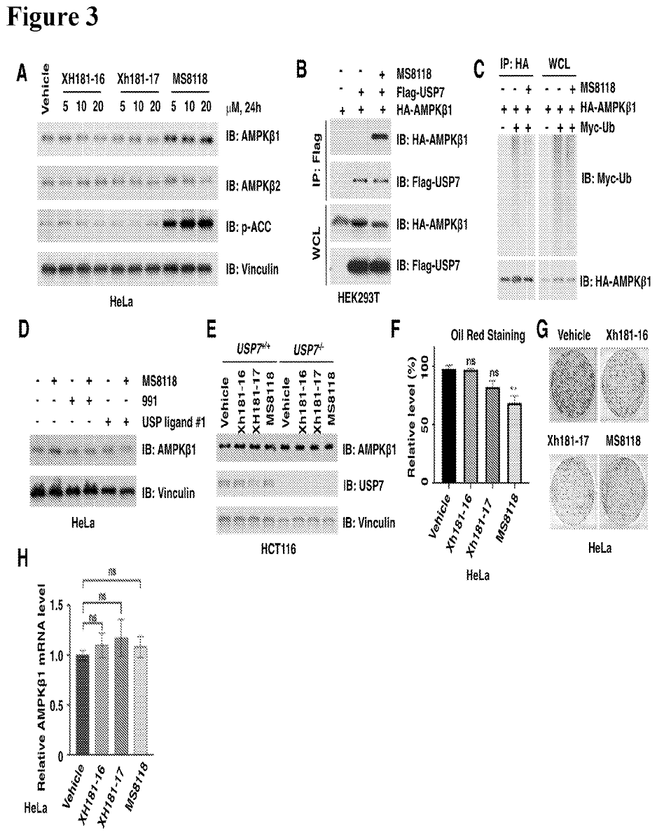

### Example 196. Assessment of the Effect of Selected AMPK Based Bivalent Compounds (XH Series) in Stabilization AMPK Protein Level and Activating its Downstream Targets in Hela Cells (FIG. 3)

(A) HeLa cells were treated with AMPK DUBTAC XH181-16, XH181-17, or XH181-18 (MS8118) for 24 h, followed by Western blot analysis. (B) Flag-USP7 and HA-AMPKβ1 were transfected in HEK293T cells, with or without treatment with 10 μM MS8118 for 24 h, followed by co-immunoprecipitation (co-IP) analysis. (C) IB analysis of HA-IP and WCL derived from HEK293 cells transfected with Myc-Ub and HA-AMPKβ1. Cells were treated with or without 20 μM MS8118 for 24 h and 30 μM MG132 for 8 h before harvesting. (D) HeLa cells were treated with 10 μM MS8118, together with AMPK agonist 991, or USP7 ligand #1 for 24 h, followed by Western blot analysis. (E) USP7+/+ and USP7−/− HCT116 cells were treated with AMPK DUBTAC XH181-16, XH181-17, or XH181-18 (MS8118) for 24 h. (F) HeLa cells were treated with 10 μM AMPK DUBTAC XH181-16, XH181-17, or XH181-18 (MS8118) for 24 h, followed by Oil Red O staining. n=3. **: p<0.01. ns: not significant. (G) Representative images of the colony formation assay. HeLa cells were treated with 10 μM AMPK DUBTAC XH181-16, XH181-17, or XH181-18 (MS8118) for 14 days and then subjected to crystal violet staining. n=3. (H) AMPK DUBTAC XH181-16, XH181-17, or XH181-18 (MS8118) do not affect the mRNA levels of AMPKβ1 in cells. Hela cells were treated with 10 μM indicated compounds for 24 h, followed by RT-qPCR analysis for the mRNA levels of AMPKβ1.

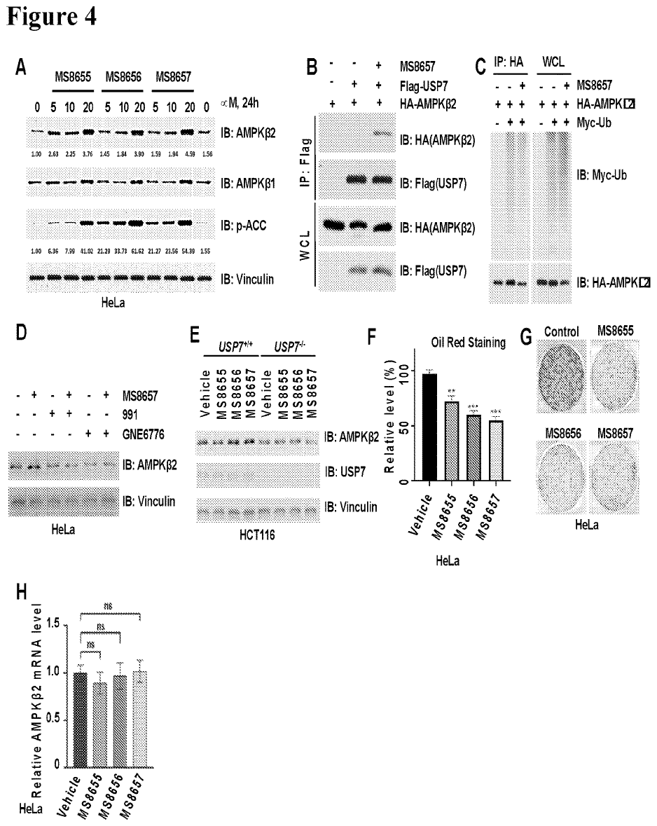

### Example 197. Assessment of the Effect of Selected AMPK Based Bivalent Compounds (KL Series) in Stabilization AMPK Protein Level and Activating its Downstream Targets in Hela Cells (FIG. 4)

(A) HeLa cells were treated with the indicated concentrations of MS8655, MS8656, and MS8657 for 24 h, followed by Western blot analysis. (B) Flag-USP7 and HA-AMPKβ2 were transfected in HEK293T cells, with or without treatment with 10 μM MS8657 for 24 h, followed by co-immunoprecipitation (co-IP) analysis. (C) IB analysis of HA-IP and WCL derived from HEK293 cells transfected with Myc-Ub and HA-AMPKβ2. Cells were treated with or without 20 μM MS8657 for 24 h and 30 μM proteasome inhibitor MG132 for 8 h before harvesting. (D) HeLa cells were treated with 10 μM MS8657, together with AMPK agonist 991, or GNE6776 for 24 h, followed by Western blot analysis. (E) USP7+/+ and USP7−/− HCT116 cells were treated with 10 μM MS8655, MS8656, or MS8657 for 24 h. (F) HeLa cells were treated with 10 M DUBTAC for 24 h, followed by Oil Red staining. n=3. **: p<0.01. ***: p<0.001. (G) Representative images of the colony formation assay. HeLa cells were treated with 10 μM MS8655, MS8656, or MS8657 for 14 days, followed by crystal violet staining. n=3. (H) AMPK DUBTAC MS8655, MS8656 and MS8657 do not affect the mRNA levels of AMPKβ2 in cells. Hela cells were treated with 10 μM indicated compounds for 24 h, followed by RT-qPCR analysis for the mRNA levels of AMPKβ2.

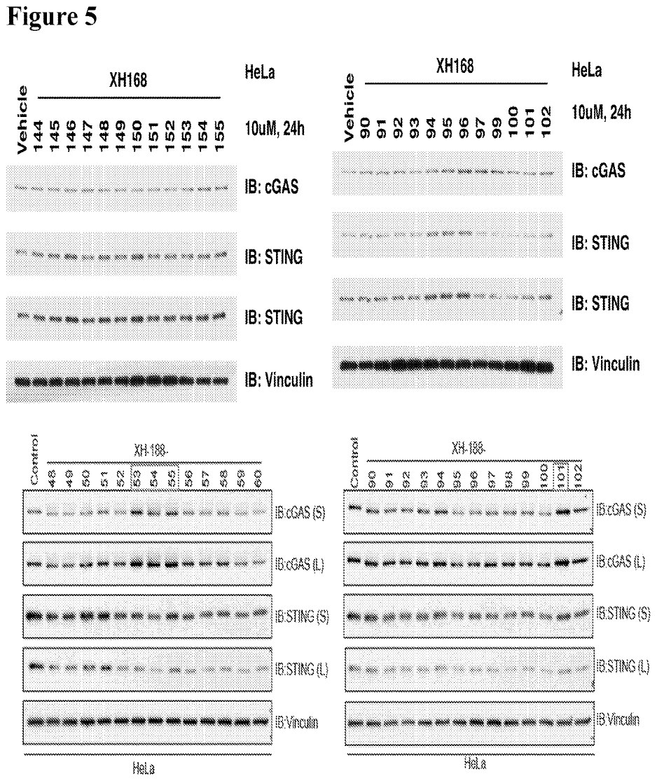

### Example 198. Assessment of the Effect of Exemplary cGAS Based Bivalent Compounds (XH Series) on Stabilization of the cGAS Protein Level in Hela Cells (FIG. 5)

The Hela cells was treated with DMSO, or selected bivalent compound at 10 μM for 24 h. The Western blot results showed that multiple compounds can increase cGAS and its downstream target STING proteins level.

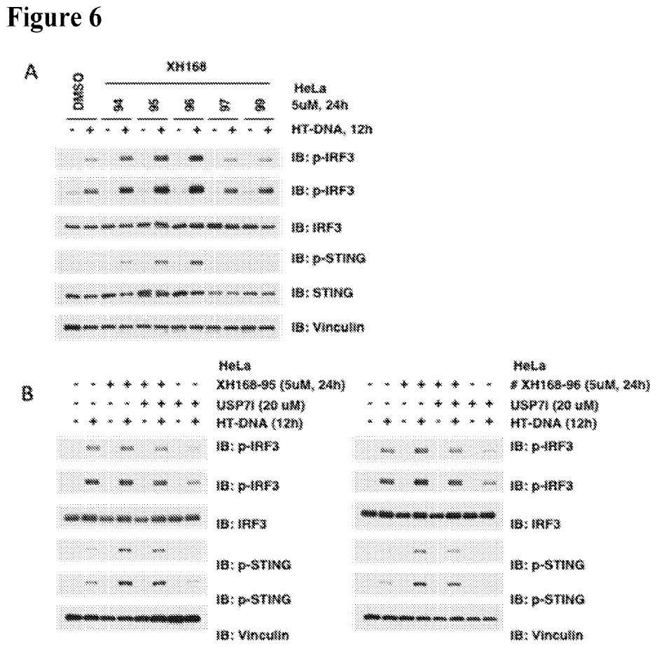

### Example 199. Assessment of the Effect of Selected cGAS Based Bivalent Compounds (XH Series) in Stabilization cGAS Protein Level and Activating its Downstream Targets with or without DNA Stimulation. (FIG. 6)

(A) The Hela cells was treated with DMSO, or selected bivalent compound at 5 μM for 24 h with or without DNA stimulation (20 μM). The western blot results showed that several compounds can effectively increase cGAS and its downstream target STING proteins level and activate the phosphorylation of STING and IRF3 under DNA stimulation. (B) Pretreatment with USP7 ligand (20 μM) can reverse the stabilization and activation effect of selected bivalent compounds.

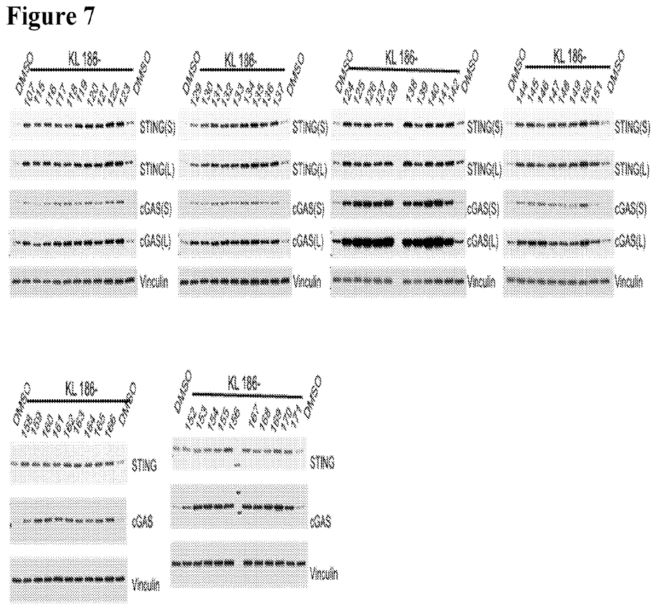

### Example 200. Assessment of the Effect of Exemplary cGAS Based Bivalent Compounds (KL Series) on Stabilization of the cGAS Protein Level in Hela Cells (FIG. 7)

The Hela cells was treated with DMSO, or selected bivalent compound at 10 μM for 24 h. The Western blot results showed that multiple compounds can increase cGAS and its downstream target STING proteins level.

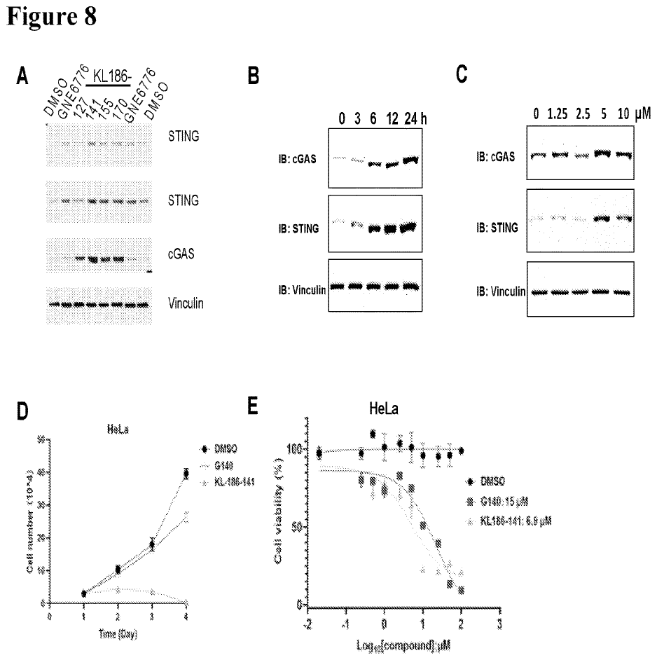

### Example 201. Assessment of the Effect of Selected Exemplary cGAS Based Bivalent Compounds (KL Series) in Stabilization cGAS Protein Level and Antiproliferation Effect in Hela Cells. (FIG. 8)

(A) The Hela cells were treated with DMSO, USP7 inhibitor GNE6776 or selected bivalent compound at 5 μM for 24 h. The western blot results showed that several compounds can effectively increase cGAS and its downstream target STING protein levels. (B) KL186-141 increased cGAS and its downstream target STING protein levels in time-dependent manner. Hela cells were treated with KL186-141 (10 μM) at indicated time. (C) KL186-141 increased cGAS and its downstream target STING protein levels in concentration-dependent manner. Hela cells were treated with KL186-141 at indicated concentrations for 24 h. (D) KL186-141, but not cGAS agonist G140 effective suppressed Hela cells proliferation. Hela cells were treated with DMSO, G140 or KL186-141 (10 M). (E) IC₅₀ determination of KL186-141 in Hela cells. Hela cells were treated with DMSO, G140 or KL186-141 at indicated concentrations for 3 days.

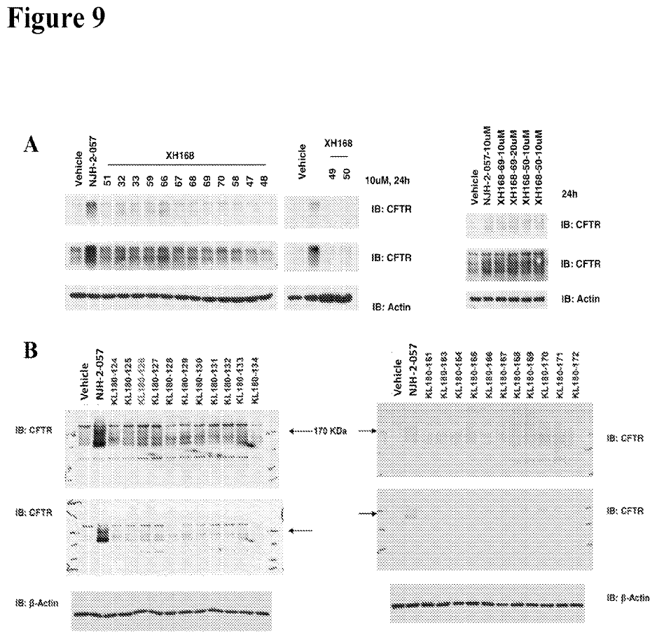

### Example 202. Assessment of the Effect of Exemplary CFTR Based Bivalent Compounds in Stabilization CFTR Protein Level in Human Cystic Fibrosis Bronchial Epithelial Cells (FIG. 9)

(A) Left, the CFBE41o− 4.7 ΔF508-CFTR human cystic fibrosis bronchial epithelial cells were treated with DMSO, positive control (NJH-2-057) or selected bivalent compound at 10 μM for 24 h. The Western blot results showed that multiple compounds can increase CFTR protein level; Right, the CFBE41o− 4.7 ΔF508-CFTR human cystic fibrosis bronchial epithelial cells were treated with DMSO, positive control (NJH-2-057) or selected bivalent compound at 10 or 20 μM for 24 h. (b) CFBE41o− 4.7 ΔF508-CFTR human cystic fibrosis bronchial epithelial cells was treated with DMSO, positive control (NJH-2-057) or selected bivalent compound at indicated concentrations (10 μM) for 24 h. The Western blot results showed that the compounds can effectively increase CFTR protein level.

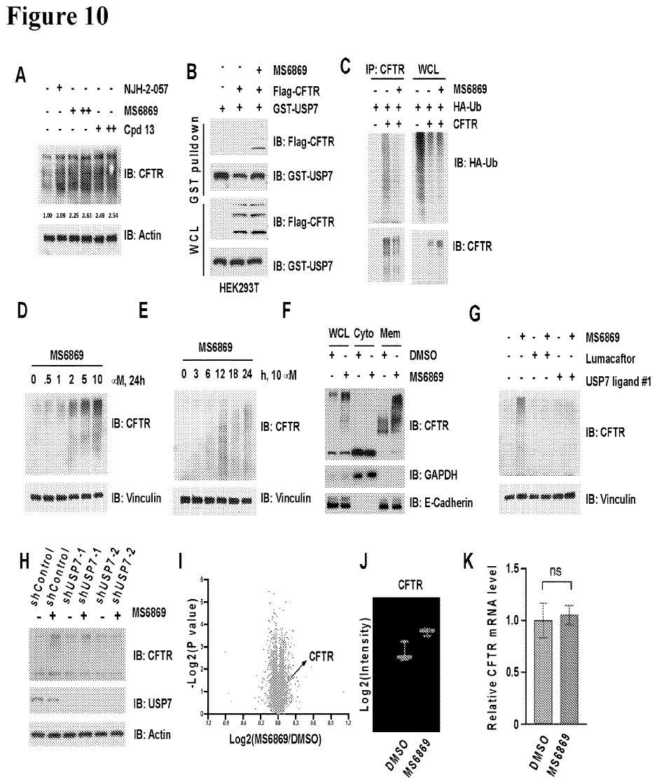

### Example 203. Assessment of the Effect of Selected Exemplary CFTR Based Bivalent Compound in Stabilization CFTR Protein Level in Human Cystic Fibrosis Bronchial Epithelial Cells and Related Mechanism of Action. (FIG. 10)

(A) The human cystic fibrosis bronchial epithelial CFBE41o− 4.7 ΔF508-CFTR cells were treated with 10 or 20 μM of the indicated compounds for 24 h, with NJH-2-057 as a positive control, followed by Western blot analysis. (B) Flag-CFTR and GST-USP7 were transfected into HEK293T cells, with or without treatment with 10 μM MS6869 for 24 h, followed by the GST pulldown assay. (C) IB analysis of CFTR-IP and WCL derived from HEK293 cells transfected with HA-Ub and CFTR expression plasmids. Cells were treated with or without 20 μM MS8118 for 24 h and 30 μM proteasome inhibitor MG132 for 8 h before harvesting. (D) CFBE41o− 4.7 ΔF508-CFTR cells were treated with the indicated concentrations of MS6869 for 24 h, followed by Western blot analysis. (E) CFBE41o− 4.7 ΔF508-CFTR cells were treated with 10 μM MS6869 for the indicated hours, followed by Western blot analysis. (F) CFBE41o− 4.7 ΔF508-CFTR cells were treated with 10 μM MS6869 for 24 h, followed by the cellular component fraction for cytoplasm (Cyto) and plasma membrane (Mem). (G) CFBE41o− 4.7 ΔF508-CFTR cells were treated with MS6869, with or without the co-treatment with USP7 ligand #1 or CFTR ligand Lumacaftor for 24 h, followed by Western blot analysis. (H) CFBE41o− 4.7 ΔF508-CFTR cells were depleted of USP7 and then treated with 10 μM MS6869 for 24 h, followed by Western blot analysis. (I-J) CFBE41o− 4.7 ΔF508-CFTR cells were treated with 10 μM of MS6869 for 24 h, followed by TMT-labeling-based mass spectrometry analysis. CFTR was highlighted in the volcano plot with boxplot showing CFTR protein abundance in DMSO and MS6869-treated cells. Results from three biological replicates (n=3) and unpaired t test (*p<0.05) are shown. (K) CFTR DUBTAC MS6869 do not affect the mRNA levels of CFTR in cells. CFBE41o− 4.7 ΔF508-CFTR cells were treated with 10 μM of MS6869 for 24 h, followed by RT-qPCR analysis for the mRNA levels of CFTR.

### Materials and Methods

### Cell Culture

The CFBE41o− 4.7 ΔF508-CFTR human cystic fibrosis bronchial epithelial cells were purchased from Millipore Sigma (SCC159). CFBE41o− 4.7 ΔF508-CFTR cells are derived from the parental CFBE41o− cells by introduced the ΔF508-CFTR construct. CFBE41o− 4.7 ΔF508-CFTR cells were cultured in α-MEM media (Sigma. #M2279) supplemented with 10% FBS, 2 mM L-Glutamine, 300 μg/mL Hygromycin B, 10,000 units/mL Penicillin and 10,000 μg/mL Streptomycin. For treatment, cells were plated in 6-well plate which is pre-coated with coating mixture (10 μg/mL Fibronectin (Sigma, #F2006), 30 μg/mL PureCol Collagen (Sigma, #5006), and 100 μg/mL BSA (Sigma. #126575)), and treated with indicated drugs when cells reach 60-70% confluency. Hela cells were cultured in Dulbecco's Modified Eagle's Medium (DMEM) containing 10% fetal bovine serum (FBS), 100 Units of penicillin and 100 μg/ml streptomycin. For treatment, cells in 6-well plate were treated with indicated compounds. To activate the cGAS/STING pathways, cells were transfected with either HT-DNA (Sigma, D6898) with PEI (Polysciences, #23966). For free ligand competition assay, the free ligand, USP7 ligand, cGAS ligand or AMPK ligand in DMSO were co-treated with indicated DUBTAC compounds.

### Western Blot

Cells were lysed in EBC buffer (120 mM NaCl, 50 mM Tris-Cl pH 8.0, 0.5% NP-40) supplemented with protease inhibitors cocktail (Pierce) and phosphatase inhibitors (phosphatase inhibitor cocktail set I and II, Calbiochem). The protein samples were resolved by 7% SDS-PAGE at 130 V for 80 min, and immunoblotted with indicated antibodies at 4° C. overnight, washed four times with Tris-buffered saline with 0.1% Tween-20 (TBST) buffer, incubated with secondary antibody in 5% non-fat milk for 1 hour at room temperature, and then washed another four times with TBST buffer. CFTR (#78335, 1:1,000) antibody was purchased from Cell Signaling Technologies. Anti-Actin (A2228, 1:50,000), anti-vinculin antibody (V-4505, 1:50,000), peroxidase-conjugated anti-mouse secondary antibody (A-4416, 1:3000) and peroxidase-conjugated anti-rabbit secondary antibody (A-4914, 1:3,000) were purchased from Sigma. All primary antibodies were diluted in 5% bovine serum albumin (BSA) in TBST buffer, and secondary antibodies were diluted in 5% non-fat milk in TBST buffer.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.
Aledavood, E., Gheeraert, A., Forte, A., Vuillon, L., Rivalta, I., Luque, F. J., and Estarellas, C. (2021). Elucidating the activation mechanism of AMPK by direct pan-activator PF-739. Front. Mol. Biosci. 8, 760026. 10.3389/fmolb.2021.760026.
Colland, F., Formstecher, E., Jacq, X., Reverdy, C., Planquette, C., Conrath, S., Trouplin, V., Bianchi, J., Aushev, V. N., Camonis, J., et al. (2009). Small-molecule inhibitor of USP7/HAUSP ubiquitin protease stabilizes and activates p53 in cells. Mol. Cancer. Ther. 8, 2286-2295. 10.1158/1535-7163.Mct-09-0097.
Cusi, K., Alkhouri, N., Harrison, S. A., Fouqueray, P., Moller, D. E., Hallakou-Bozec, S., Bolze, S., Grouin, J. M., Jeannin Megnien, S., Dubourg, J., and Ratziu, V. (2021). Efficacy and safety of PXL770, a direct AMP kinase activator, for the treatment of non-alcoholic fatty liver disease (STAMP-NAFLD): a randomised, double-blind, placebo-controlled, phase 2a study. Lancet Gastroenterol Hepatol. 6, 889-902. 10.1016/s2468-1253 (21) 00300-9.
Dale, B., Anderson, C., Park, K.-S., Kaniskan, H. Ü., Ma, A., Shen, Y., Zhang, C., Xie, L., Chen, X., Yu, X., and Jin, J. (2022). Targeting Triple-Negative Breast Cancer by a Novel Proteolysis Targeting Chimera Degrader of Enhancer of Zeste Homolog 2. ACS Pharmacol. Transl. Sci. 5, 491-507. 10.1021/acsptsci.2c00100.
Di Lello, P., Pastor, R., Murray, J. M., Blake, R. A., Cohen, F., Crawford, T. D., Drobnick, J., Drummond, J., Kategaya, L., Kleinheinz, T., et al. (2017). Discovery of small-molecule inhibitors of ubiquitin specific protease 7 (USP7) using integrated NMR and in silico techniques. J. Med. Chem. 60, 10056-10070. 10.1021/acs.jmedchem.7b01293.
Edmonds, D. J., Kung, D. W., Kalgutkar, A. S., Filipski, K. J., Ebner, D. C., Cabral, S., Smith, A. C., Aspnes, G. E., Bhattacharya, S. K., Borzilleri, K. A., et al. (2018). Optimization of metabolic and renal clearance in a series of indole acid direct activators of 5′-Adenosine Monophosphate-Activated Protein Kinase (AMPK). J. Med. Chem. 61, 2372-2383. 10.1021/acs.jmedchem.7b01641.
Figarola, J. L., and Rahbar, S. (2013). Small-molecule COH—SR4 inhibits adipocyte differentiation via AMPK activation. Int. J. Mol. Med. 31, 1166-1176. 10.3892/ijmm.2013.1313.
Gavory, G., O'Dowd, C. R., Helm, M. D., Flasz, J., Arkoudis, E., Dossang, A., Hughes, C., Cassidy, E., McClelland, K., Odrzywol, E., et al. (2018). Discovery and characterization of highly potent and selective allosteric USP7 inhibitors. Nat. Chem. Biol. 14, 118-125. 10.1038/nchembio.2528.
Henning, N. J., Boike, L., Spradlin, J. N., Ward, C. C., Liu, G., Zhang, E., Belcher, B. P., Brittain, S. M., Hesse, M. J., Dovala, D., et al. (2022). Deubiquitinase-targeting chimeras for targeted protein stabilization. Nat. Chem. Biol. 18, 412-421. 10.1038/s41589-022-00971-2.
Hou, D., Yuan, B., Duan, X., Wang, J., Min, W., Chen, S., Liu, X., Liu, S., and Sun, Y. (2022). Usp7 inhibitor. patent application WO2022048498.
Jeon, S. M. (2016). Regulation and function of AMPK in physiology and diseases. Exp. Mol. Med. 48, e245. 10.1038/emm.2016.81.
Kategaya, L., Di Lello, P., Rouge, L., Pastor, R., Clark, K. R., Drummond, J., Kleinheinz, T., Lin, E., Upton, J.-P., Prakash, S., et al. (2017). USP7 small-molecule inhibitors interfere with ubiquitin binding. Nature 550, 534-538. 10.1038/nature24006.
Kim, Y. M., Kim, M. Y., Kim, H. J., Roh, G. S., Ko, G. H., Seo, H. G., Lee, J. H., and Chang, K. C. (2011). Compound C independent of AMPK inhibits ICAM-1 and VCAM-1 expression in inflammatory stimulants-activated endothelial cells in vitro and in vivo. Atherosclerosis 219, 57-64. 10.1016/j.atherosclerosis.2011.06.043.
Kotschy, A., Weber, C., Vasas, A., Molnar, B., Kiss, A., Macias, A., Murray, J. B., Lewkowicz, E., Geneste, O., and Chanrion, M. (2017). New heteroaryl-substituted-piperidinyl derivatives, a process for their preparation and pharmaceutical compositions containing them. patent application WO2017212010.
Lama, L., Adura, C., Xie, W., Tomita, D., Kamei, T., Kuryavyi, V., Gogakos, T., Steinberg, J. I., Miller, M., Ramos-Espiritu, L., et al. (2019). Development of human cGAS-specific small-molecule inhibitors for repression of dsDNA-triggered interferon expression. Nat. Comm. 10, 2261. 10.1038/s41467-019-08620-4.
Lamberto, I., Liu, X., Seo, H.-S., Schauer, N. J., lacob, R. E., Hu, W., Das, D., Mikhailova, T., Weisberg, E. L., Engen, J. R., et al. (2017). Structure-guided development of a potent and selective non-covalent active-Site inhibitor of USP7. Cell Chem. Biol. 24, 1490-1500.e1411.
Leger, P. R., Hu, D. X., Biannic, B., Bui, M., Han, X., Karbarz, E., Maung, J., Okano, A., Osipov, M., Shibuya, G. M., et al. (2020). Discovery of potent, selective, and orally bioavailable inhibitors of USP7 with in antitumor vivo activity. J. Med. Chem. 63, 5398-5420. 10.1021/acs.jmedchem.0c00245.
Lemos, C., Schulze, V. K., Baumgart, S. J., Nevedomskaya, E., Heinrich, T., Lefranc, J., Bader, B., Christ, C. D., Briem, H., Kuhnke, L. P., et al. (2021). The potent AMPK inhibitor BAY-3827 shows strong efficacy in androgen-dependent prostate cancer models. Cell Oncol (Dordr) 44, 581-594. 10.1007/s13402-020-00584-8.
Li, M., Liu, S., Chen, H., Zhou, X., Zhou, J., Zhou, S., Yuan, H., Xu, Q.-L., Liu, J., Cheng, K., et al. (2020). N-benzylpiperidinol derivatives as novel USP7 inhibitors: Structure-activity relationships and X-ray crystallographic studies. Eur. J. Med. Chem. 199, 112279.
Liu, J., Yu, X., Chen, H., Kaniskan, H. Ü., Xie, L., Chen, X., Jin, J., and Wei, W. (2022). TF-DUBTACs stabilize tumor suppressor transcription factors. J. Am. Chem. SoC 144, 12934-12941. 10.1021/jacs.2c04824.
Myers, R. W., Guan, H. P., Ehrhart, J., Petrov, A., Prahalada, S., Tozzo, E., Yang, X., Kurtz, M. M., Trujillo, M., Gonzalez Trotter, D., et al. (2017). Systemic pan-AMPK activator MK-8722 improves glucose homeostasis but induces cardiac hypertrophy. Science 357, 507-511. 10.1126/science.aah5582.
Nakamaru, K., Matsumoto, K., Taguchi, T., Suefuji, M., Murata, Y., Igata, M., Kawashima, J., Kondo, T., Motoshima, H., Tsuruzoe, K., et al. (2005). AICAR, an activator of AMP-activated protein kinase, down-regulates the insulin receptor expression in HepG2 cells. Biochem. Biophys. Res. Commun. 328, 449-454. 10.1016/j.bbrc.2005.01.004.
Ngoei, K. R. W., Langendorf, C. G., Ling, N. X. Y., Hoque, A., Varghese, S., Camerino, M. A., Walker, S. R., Bozikis, Y. E., Dite, T. A., Ovens, A. J., et al. (2018). Structural determinants for small-molecule activation of skeletal muscle AMPK α2β2γ1 by the glucose importagog SC4. Cell Chem. Biol. 25, 728-737.e729. 10.1016/j.chembiol.2018.03.008.
Nicholson, B., Leach, C. A., Goldenberg, S. J., Francis, D. M., Kodrasov, M. P., Tian, X., Shanks, J., Sterner, D. E., Bernal, A., Mattern, M. R., et al. (2008). Characterization of ubiquitin and ubiquitin-like-protein isopeptidase activities. Protein Sci. 17, 1035-1043. 10.1110/ps.083450408.
O'Dowd, C., Harrison, T., Hewitt, P., Rountree, S., Hugues, M., Burkamp, F., Jordan, L., Helm, M., Broccatelli, F., and Crawford, J. J. (2018). Preparation of hydroxypiperidinyl ketones as inhibitors of ubiquitin specific protease 7 for treatment of cancer. patent application WO2018073602.
Pepe, A., and Mesecar, A. (2018). Preparation of 6-amino-2,4-dihydropyrano[2,3-c]pyrazoles as inhibitors of USP7 for use in diseases involving USP7 malfunction such as inflammation, cancer, and immunological disorders. patent application WO2018183587.
Reverdy, C., Conrath, S., Lopez, R., Planquette, C., Atmanene, C., Collura, V., Harpon, J., Battaglia, V., Vivat, V., Sippl, W., and Colland, F. (2012). Discovery of specific inhibitors of human USP7/HAUSP deubiquitinating enzyme. Chem. Biol. 19, 467-477.
Rhodes, N., Heerding, D. A., Duckett, D. R., Eberwein, D. J., Knick, V. B., Lansing, T. J., McConnell, R. T., Gilmer, T. M., Zhang, S. Y., Robell, K., et al. (2008). Characterization of an Akt kinase inhibitor with potent pharmacodynamic and antitumor activity. Cancer Res. 68, 2366-2374. 10.1158/0008-5472.Can-07-5783.
Samson, N., and Ablasser, A. (2022). The cGAS-STING pathway and cancer. Nat. Cancer 3, 1452-1463. 10.1038/s43018-022-00468-w.
Sanders, M. J., Ali, Z. S., Hegarty, B. D., Heath, R., Snowden, M. A., and Carling, D. (2007). Defining the mechanism of activation of AMP-activated protein kinase by the small molecule A-769662, a member of the thienopyridone family. J. Biol. Chem. 282, 32539-32548. 10.1074/jbc.M706543200.
Schauer, N. J., Liu, X., Magin, R. S., Doherty, L. M., Chan, W. C., Ficarro, S. B., Hu, W., Roberts, R. M., lacob, R. E., Stolte, B., et al. (2020). Selective USP7 inhibition elicits cancer cell killing through a p53-dependent mechanism. Sci. Rep. 10, 5324. 10.1038/s41598-020-62076-x.
Sun, G., You, Y., Li, H., Cheng, Y., Qian, M., Zhou, X., Yuan, H., Xu, Q. L., Dai, L., Wang, P., et al. (2020). Discovery of AdipoRon analogues as novel AMPK activators without inhibiting mitochondrial complex I. Eur. J. Med. Chem. 200, 112466. 10.1016/j.ejmech.2020.112466.
Sun, H., Chen, C., Zhou, X., Liu, S., Yuan, H., and Wen, X. (2021). Benzothiazole compound and medical application as USP7 C-terminal domain regulating agent. patent application WO2021175234.
Turnbull, A. P., loannidis, S., Krajewski, W. W., Pinto-Fernandez, A., Heride, C., Martin, A. C. L., Tonkin, L. M., Townsend, E. C., Buker, S. M., Lancia, D. R., et al. (2017). Molecular basis of USP7 inhibition by selective small-molecule inhibitors. Nature 550, 481-486. 10.1038/nature24451.
Vingtdeux, V., Chandakkar, P., Zhao, H., Davies, P., and Marambaud, P. (2011). Small-molecule activators of AMP-activated protein kinase (AMPK), RSVA314 and RSVA405, inhibit adipogenesis. Mol. Med. 17, 1022-1030. 10.2119/molmed.2011.00163.
Ward, C. L., Omura, S., and Kopito, R. R. (1995). Degradation of CFTR by the ubiquitin-proteasome pathway. Cell 83, 121-127. 10.1016/0092-8674 (95) 90240-6.
Weinstock, J., Wu, J., Cao, P., Kingsbury, W. D., McDermott, J. L., Kodrasov, M. P., Mckelvey, D. M., Suresh Kumar, K. G., Goldenberg, S. J., Mattern, M. R., and Nicholson, B. (2012). Selective dual inhibitors of the cancer-related deubiquitylating proteases USP7 and USP47. ACS Med. Chem. Lett. 3, 789-792. 10.1021/ml200276j.
Xiao, D., Lu, Z., Wang, Z., Zhou, S., Cao, M., Deng, J., Hu, X., Peng, M., He, C., Wu, J., et al. (2020). Synthesis, biological evaluation and anti-proliferative mechanism of fluorine-containing proguanil derivatives. Bioorg. Med. Chem. 28, 115258. 10.1016/j.bmc.2019.115258.
Zhao, J., Xiao, R., Zeng, R., He, E., and Zhang, A. (2022). Small molecules targeting cGAS-STING pathway for autoimmune disease. E. J. Med. Chem. 238, 114480.

1. A bivalent compound comprising a ligand bound to a de-ubiquitination tag through a linker, the de-ubiquitination tag including a ubiquitin-specific-processing protease 7 (USP7) recruiter according to Formula (E-I): 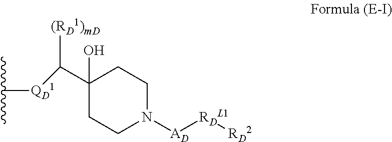
wherein [symbol]
denotes the point of attachment to the linker;
A_{D} is selected from C═O or C(R_{D}³)₂;
each R_{D}¹ and each R_{D}³ is independently selected from H, OH, C₁-C₆ alkyl and C₃-C₆ cycloalkyl, where each said C₁-C₆ alkyl and C₃-C₆ cycloalkyl is optionally substituted with halogen, oxo, OR_{D}⁴, NR_{D}⁴R_{D}⁵, C(O)OR_{D}⁴, C(O)NR_{D}⁴R_{D}⁵, OC(O)R_{D}⁶, NR_{D}⁴C(O)R_{D}⁶, S(O)₂NR_{D}⁴R_{D}⁵, NR_{D}⁴S(O)₂R_{D}⁶, C₁-C₃ alkyl, C₁-C₃ heteroalkyl, C₃-C₇ cycloalkyl, 3-7 membered heterocyclic, C₆-C₁₀ aryl, or 5-10 membered heteroaryl, or
two R_{D}¹, or two R_{D}³ together with the atom(s) to which they are attached, optionally form C₃-C₇ cycloalkyl or 3-7 membered heterocyclic;
R_{D}² is selected from hydrogen, C₁-C₆ alkyl, C₂-C₆ alkenyl, C₂-C₆ alkynyl, C₁-C₆ heteroalkyl, C₃-C₇ cycloalkyl, 3-7 membered heterocyclic, C₆-C₁₀ aryl, 5-10 membered heteroaryl, C₆-C₁₀ aryloxy, or 5-10 membered heteroaryloxy, where each said C₁-C₆ alkyl, C₂-C₆ alkenyl, C₂-C₆ alkynyl, C₁-C₆ heteroalkyl, C₃-C₇ cycloalkyl, 3-7 membered heterocyclic, C₆-C₁₀ aryl, 5-10 membered heteroaryl, C₆-C₁₀ aryloxy, 5-10 membered heteroaryloxy is optionally substituted with halogen, cyano, oxo, OR_{D}⁴, NR_{D}⁴R_{D}⁵, C(O)OR_{D}⁴, C(O)NR_{D}⁴R_{D}⁵, OC(O)R_{D}⁶, NRDC(O)R_{D}⁶, S(O)₂NR_{D}⁴R_{D}⁵, NR_{D}⁴S(O)₂R_{D}⁶, C₁-C₃ alkyl, C₁-C₃ heteroalkyl, C₃-C₇ cycloalkyl, 3-7 membered heterocyclic, C₆-C₁₀ aryl, or 5-10 membered heteroaryl;
R_{D}^{L1} is selected from a bond, C₁-C₆ alkylene, C₁-C₆ haloalkylene, C₁-C₆ heteroalkylene, C₃-C₁₂ cycloalkylene, 3-12 membered heterocyclicene, C₆-C₁₀ arylene, and 5-10 membered heteroarylene;
Q_{D}¹ is selected from nitrogen containing 5-10 membered heterocyclic and 5-10 membered heteroaryl;
each R_{D}⁴ and each R_{D}⁵ are independently selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic, or
R_{D}⁴ and R_{D}⁵, together with the atoms to which they are attached, optionally form 3-12 membered heterocyclic;
each R_{D}⁶ is selected from OR_{D}⁴, NR_{D}⁴R_{D}⁵, C₁-C₆ alkyl, C₁-C₆ haloalkyl, C₁-C₆ heteroalkyl, C₂-C₆ alkenyl, C₂-C₆ alkynyl, C₃-C₇ cycloalkyl, 3-7 membered heterocyclic, C₆-C₁₀ aryl, and 5-10 membered heteroaryl; and
m_{D} is selected from 0, 1 and 2, or a pharmaceutically acceptable salt of the compound, or stereoisomer, or a tautomer thereof.

2. The bivalent compound of claim 1, wherein Q_{D}¹ is selected from formulae Q_{D}-1, Q_{D}-2, Q_{D}-3, Q_{D}-4, Q_{D}-5, Q_{D}-6 and Q_{D}-7: 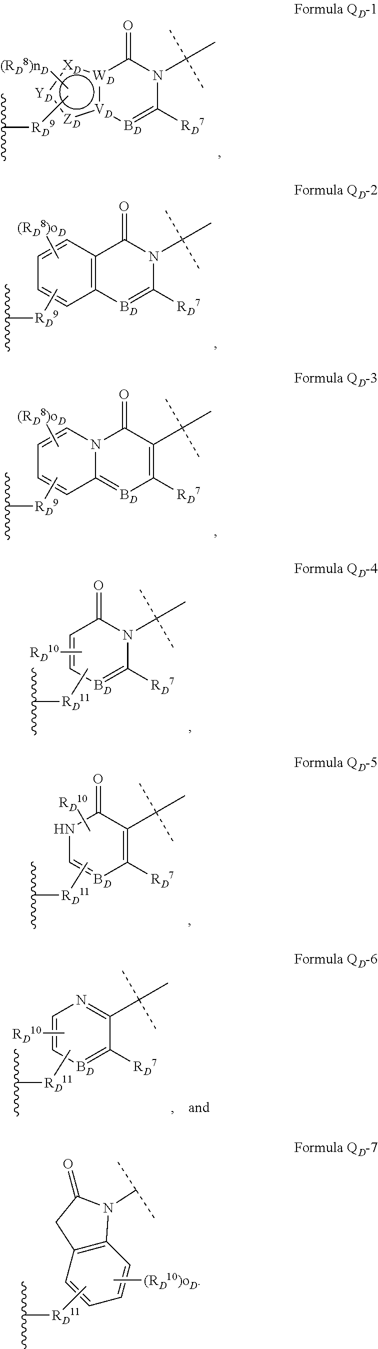
wherein
B_{D} is N or CH;
V_{D} and W_{D} are independently selected from N or C, with the proviso that at least one of V_{D} and
W_{D} is C;
X_{D}, Y_{D}, and Z_{D} are independently selected from S, O, N, NR_{D}¹², and CR_{D}⁸, with the proviso that when both V_{D} and W_{D} are C, one of X_{D}, Y_{D}, and Z_{D} is S, O, or NR_{D}¹², and when one of V_{D} and W_{D} is N, none of X_{D}, Y_{D}, and Z_{D} are S, O, or NR_{D}¹²;
R_{D}⁷ is selected from hydrogen, halogen, cyano, C₁-C₃ alkyl, C₁-C₃ haloalkyl, C₁-C₃ heteroalkyl, C₃-C₅ cycloalkyl, 3-5 membered heterocyclic;
each R_{D}⁸ and each R_{D}¹⁰ are independently selected from hydrogen, halogen, cyano, OR_{D}⁴, NR_{D}⁴R_{D}⁵, OC(O)R_{D}⁶, NR_{D}⁴C(O)R_{D}⁶, C(O)R_{D}⁶, C(O)OR_{D}⁴, C(O)NR_{D}⁴R_{D}⁵, S(O)₂NR_{D}⁴R_{D}⁵, NR_{D}⁴S(O)₂R_{D}⁶, C₁-C₆ alkyl, C₂-C₆ alkenyl, C₂-C₆ alkynyl, C₁-C₆ haloalkyl, C₁-C₆ heteroalkyl, C₃-C₁₂ cycloalkyl, 3-12 membered heterocyclic, C₆-C₁₀ aryl, and 5-10 membered heteroaryl;
each R_{D}⁹ and each R_{D}¹¹ are independently selected from —R_{D}^{L2}—R_{D}^{L3}—;
R_{D}^{L2} is selected from a bond, —O—, —NR_{D}⁴—, —C(O)—, —C(O)O—, —C(O)NR_{D}⁴—, —S(O)—, —S(O)₂—, —S(O)NR_{D}⁴—, —S(O)₂NR_{D}⁴—, C₁-C₆ alkylene, C₁-C₆ haloalkylene, C₁-C₆ heteroalkylene, C₃-C₁₂ cycloalkylene, 3-12 membered heterocyclicene, C₆-C₁₀ arylene, and 5-10 membered heteroarylene;
R_{D}^{L3} is selected from a bond, —O—, —NR_{D}⁴—, C₁-C₆ alkylene, C₁-C₆ haloalkylene, C₁-C₆ heteroalkylene, C₃-C₁₂ cycloalkylene, 3-12 membered heterocyclicene, C₆-C₁₀ arylene, and 5-10 membered heteroarylene;
R_{D}¹² is selected from hydrogen, C(O)R_{D}⁶, C(O)OR_{D}⁴, C(O)NR_{D}⁴R_{D}⁵, S(O)R_{D}⁶, S(O)₂R_{D}⁵, C₁-C₆ alkyl, C₁-C₆ haloalkyl, C₁-C₆ heteroalkyl, C₃-C₁₂ cycloalkyl, 3-12 membered heterocyclic, C₆-C₁₀ aryl, and 5-10 membered heteroaryl;
R_{D}⁴, R_{D}⁵ and R_{D}⁶ are defined in Formula (E-I);
n_{D} is selected from 0, 1 and 2; and
o_{D} is selected from 0, 1, 2, 3 and 4.

3. The bivalent compound of claim 2, wherein Q_{D}-1 is selected from formulae Q_{D}-8, Q_{D}-9, and Q_{D}-10:

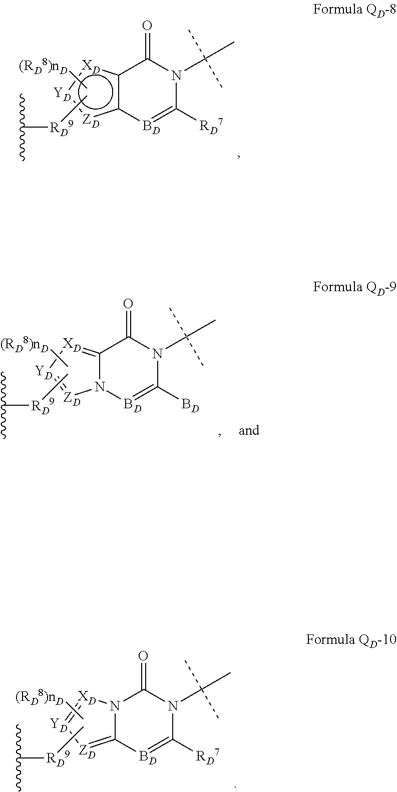

4. The bivalent compound of claim 2, wherein Q_{D}-1 is selected from formulae Q_{D}-11, Q_{D}-12, Q_{D}-13, Q_{D}-14, Q_{D}-15, Q_{D}-16, Q_{D}-17, Q_{D}-18, Q_{D}-19, Q_{D}-20, Q_{D}-21, Q_{D}-22, Q_{D}-23, Q_{D}-24, Q_{D}-25, Q_{D}-26, Q_{D}-27, Q_{D}-28, Q_{D}-29, Q_{D}-30, Q_{D}-31, Q_{D}-32, Q_{D}-33, Q_{D}-34, Q_{D}-35, and Q_{D}-36:

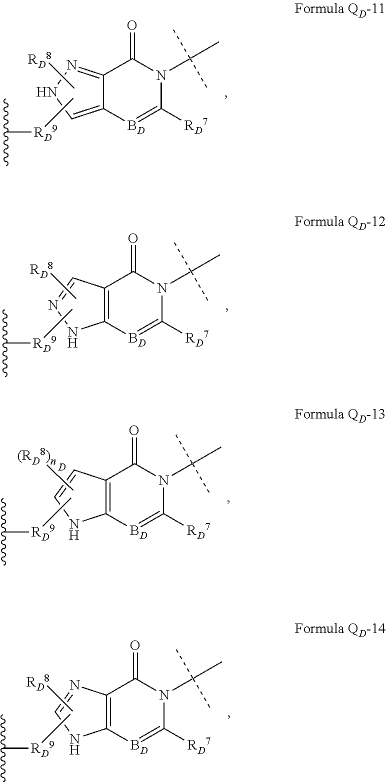

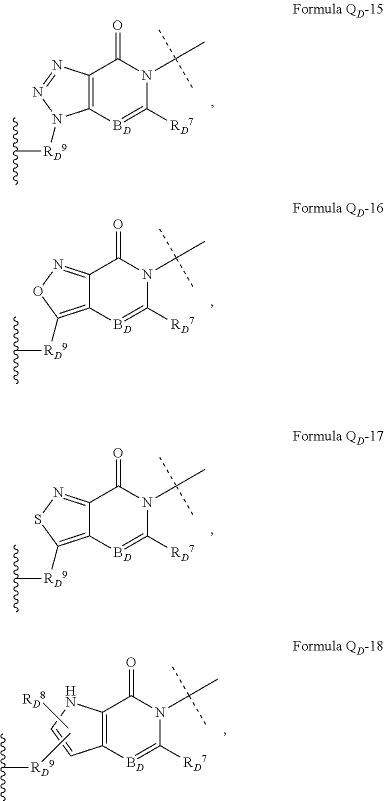

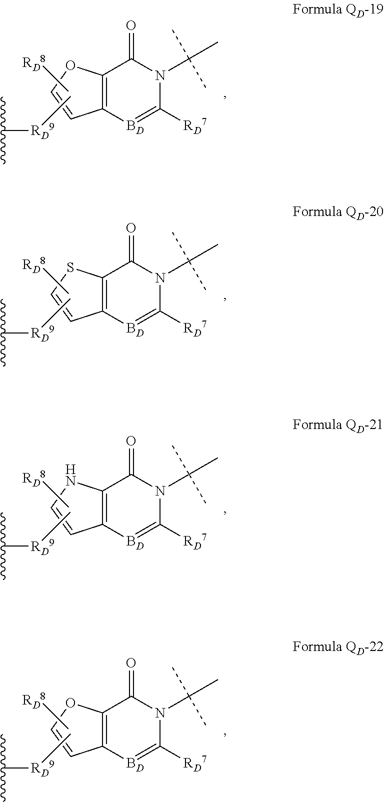

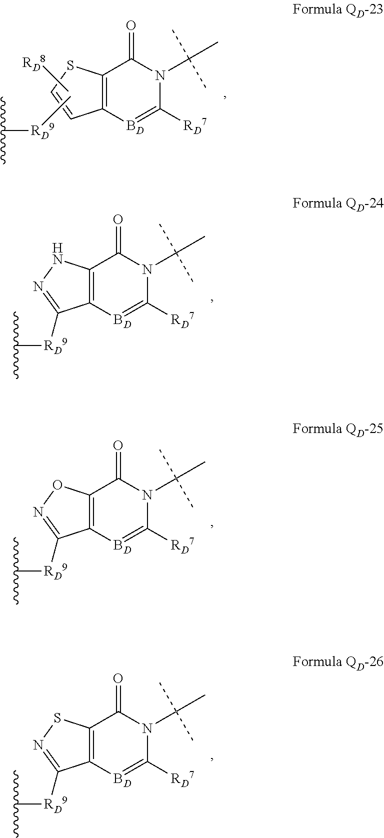

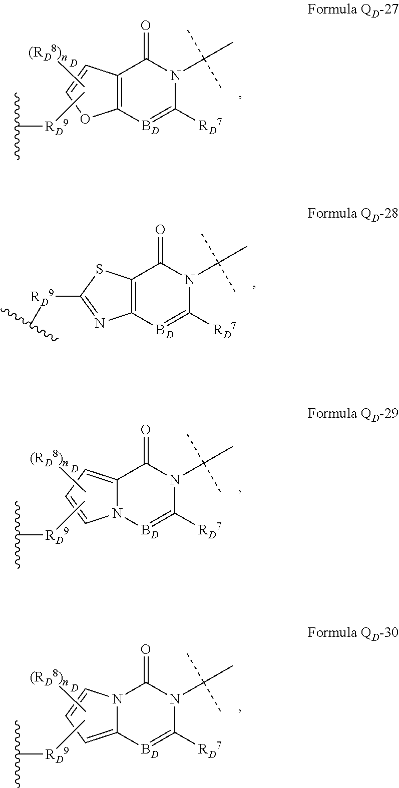

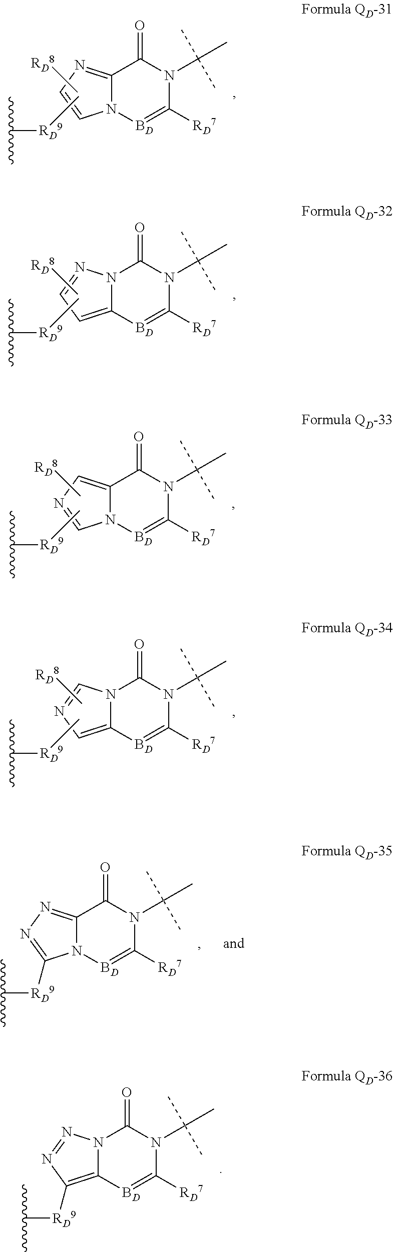

5. The bivalent compound of claim 2, wherein Q_{D}¹ is selected from formulae Q_{D}-43, Q_{D}-44, Q_{D}-45, Q_{D}-46, Q_{D}-47, Q_{D}-48:

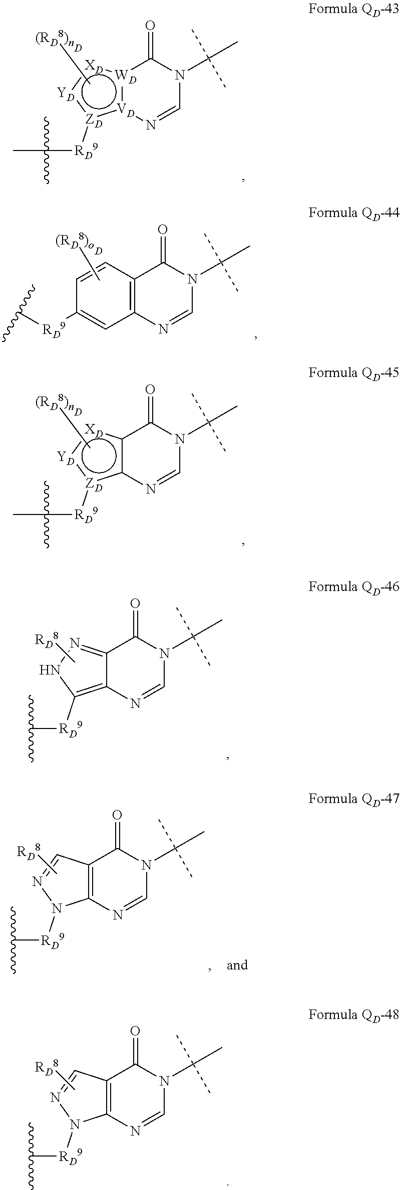

6. The bivalent compound of claim 1 wherein the USP7 recruiter has the structure of formula (E-I-a):

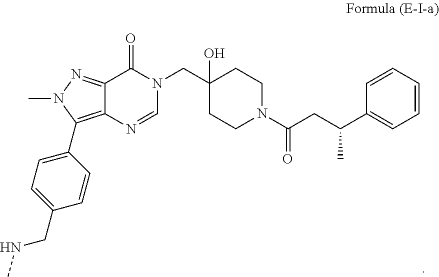

7. A bivalent compound comprising a ligand bound to a de-ubiquitination tag through a linker, the de-ubiquitination tag including a ubiquitin-specific-processing protease 7 (USP7) recruiter according to Formula (F—I): 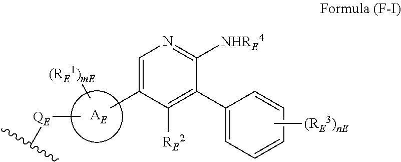
wherein [symbol]
denotes the point of attachment to the linker;
Ring A_{E} is selected from C₃-C₁₂ cycloalkylene, 3-12 membered heterocyclicene, C₆-C₁₀ arylene, and 5-10 membered heteroarylene;
Q_{E} is selected from a bond, —O—, —NR_{E}⁵—, —C(O)—, —C(O)O—, —C(O)NR_{E}⁵—, —S(O)—, —S(O)₂—, —S(O)NR_{E}⁵—, —S(O)₂NR_{E}⁵—, C₁-C₆ alkylene, C₁-C₆ haloalkylene, C₁-C₆ heteroalkylene, C₃-C₁₂ cycloalkylene, 3-12 membered heterocyclicene, C₆-C₁₀ arylene, and 5-10 membered heteroarylene;
each R_{E}¹ and each R_{E}³ are independently selected from H, halogen, cyano, CF₃, NO₂, OR_{E}⁵, NR_{E}⁵R_{E}⁶, C(O)OR_{E}⁵, C(O)NR_{E}⁵R_{E}⁶, S(O)₂NR_{E}⁵R_{E}⁶, OC(O)R_{E}⁷, NR_{E}⁵C(O)R_{E}⁷, NR_{E}⁵S(O)₂R_{E}⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR_{E}⁸, NR_{E}⁸R_{E}⁹, C(O)OR_{E}⁸, C(O)NR_{E}⁸R_{E}⁹, OP(O)(OH)₂, OP(O)(OR_{E}⁸) (OR_{E}⁹), S(O)₂R_{E}¹⁰, S(O)₂NR_{E}⁸R_{E}⁹, OC(O)R_{E}¹⁰, NR_{E}⁸C(O)R_{E}¹⁰, NR_{E}⁸S(O)₂R_{E}¹⁰, C₁-C₆ alkyl, C₁-C₆ haloalkyl, C₁-C₆ heteroalkyl, C₃-C₁₂ cycloalkyl, 3-12 membered heterocyclic, C₆-C₁₀ aryl, and 5-10 membered heteroaryl, or
two R_{E}¹, or two R_{E}³, together with the atom(s) to which they are attached, optionally form C₃-C₆ cycloalkyl or 4-7 membered heterocyclic, where each said C₃-C₆ cycloalkyl or 4-7 membered heterocyclic is optionally substituted with halogen or C₁-C₆ alkyl;
R_{E}² is selected from H, cyano, C₁-C₃ alkyl, C₁-C₃ heteroalkyl, or C₃-C₆ cycloalkyl, where each said C₁-C₃ alkyl, C₁-C₃ heteroalkyl, and C₃-C₆ cycloalkyl, is optionally substituted with halogen, especially F;
R_{E}⁴ is selected from H, C₁-C₃ alkyl, C₁-C₃ heteroalkyl, or C₃-C₆ cycloalkyl, where each said C₁-C₃ alkyl, C₁-C₃ heteroalkyl, and C₃-C₆ cycloalkyl, is optionally substituted with halogen;
each R_{E}⁵, each R_{E}⁶, each R_{E}⁸, and each R_{E}⁹, are independently selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic, or
R_{E}⁵ and R_{E}⁶, or R_{E}⁸ and R_{E} together with the atom(s) to which they are attached, optionally form 3-12 membered heterocyclic;
each R_{E}⁷, and each R_{E}¹⁰ are independently selected from ORES, NR_{E}⁵R_{E}⁶, C₁-C₆ alkyl, C₁-C₆ haloalkyl, C₁-C₆ heteroalkyl, C₂-C₆ alkenyl, C₂-C₆ alkynyl, C₃-C₇ cycloalkyl, 3-7 membered heterocyclic, C₆-C₁₀ aryl, and 5-10 membered heteroaryl;
m_{E} is selected from 0, 1, 2, 3, 4, and 5; and
n_{E} is selected from 0, 1, 2, 3, 4, 5, and 6, or a tautomer, stereoisomers, or a pharmaceutically acceptable salt of the compound thereof.

8. The bivalent compound of claim 7, wherein the USP7 recruiter comprises the structure of Formulae (F—I-a1), (F—I-a2) or (F—I-b1):

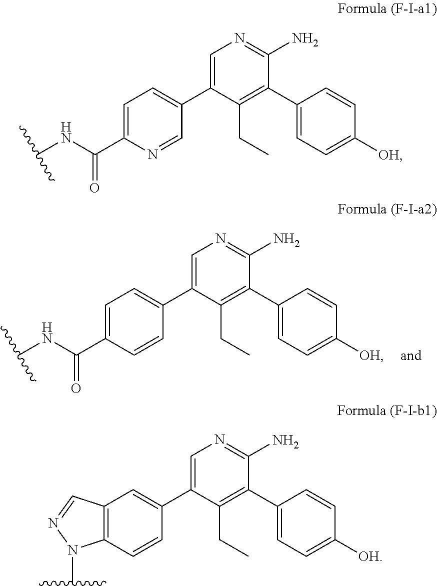 [SMILES: *NC(=O)c1ccc(-c2cnc(N)c(-c3ccc(O)cc3)c2CC)cn1 ; *NC(=O)c1ccc(-c2cnc(N)c(-c3ccc(O)cc3)c2CC)cc1 ; "and" ; *n1ncc2cc(-c3cnc(N)c(-c4ccc(O)cc4)c3CC)ccc21]

9. A bivalent compound comprising a ligand bound to a de-ubiquitination tag through a linker, the de-ubiquitination tag including a ubiquitin-specific-processing protease 7 (USP7) recruiter selected from derivatives of the following compounds: 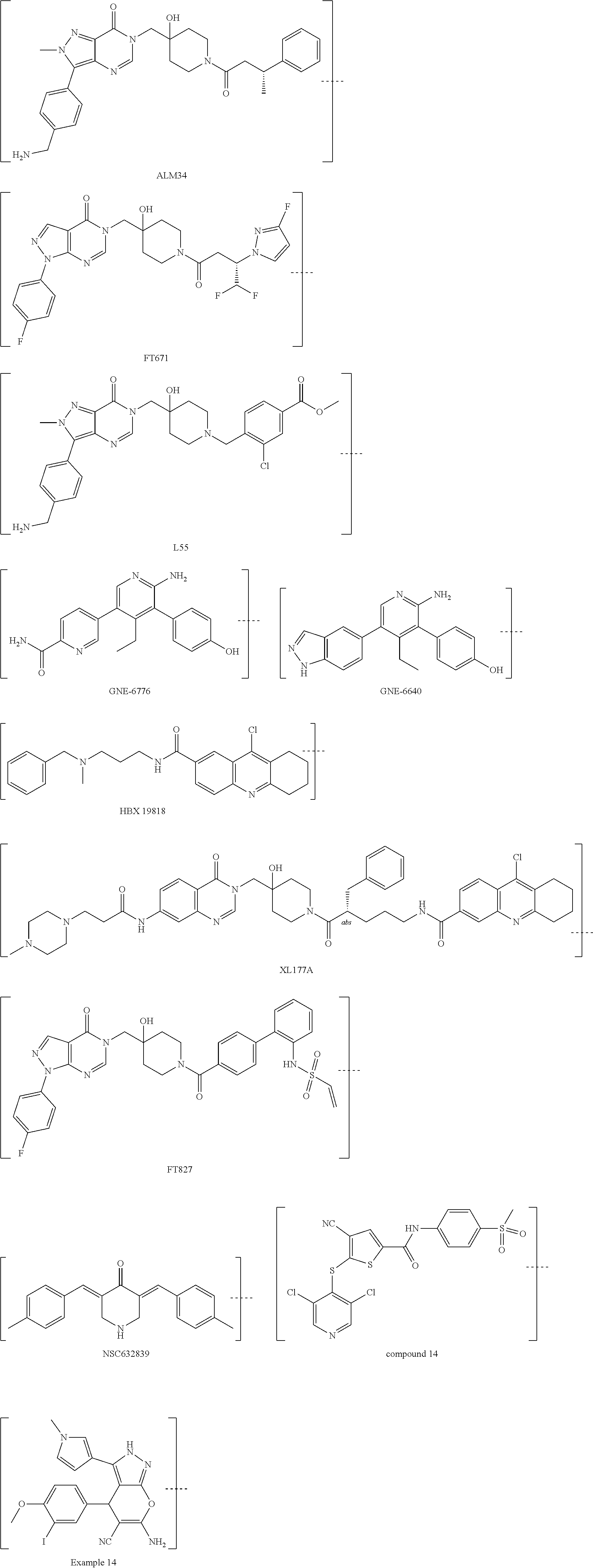 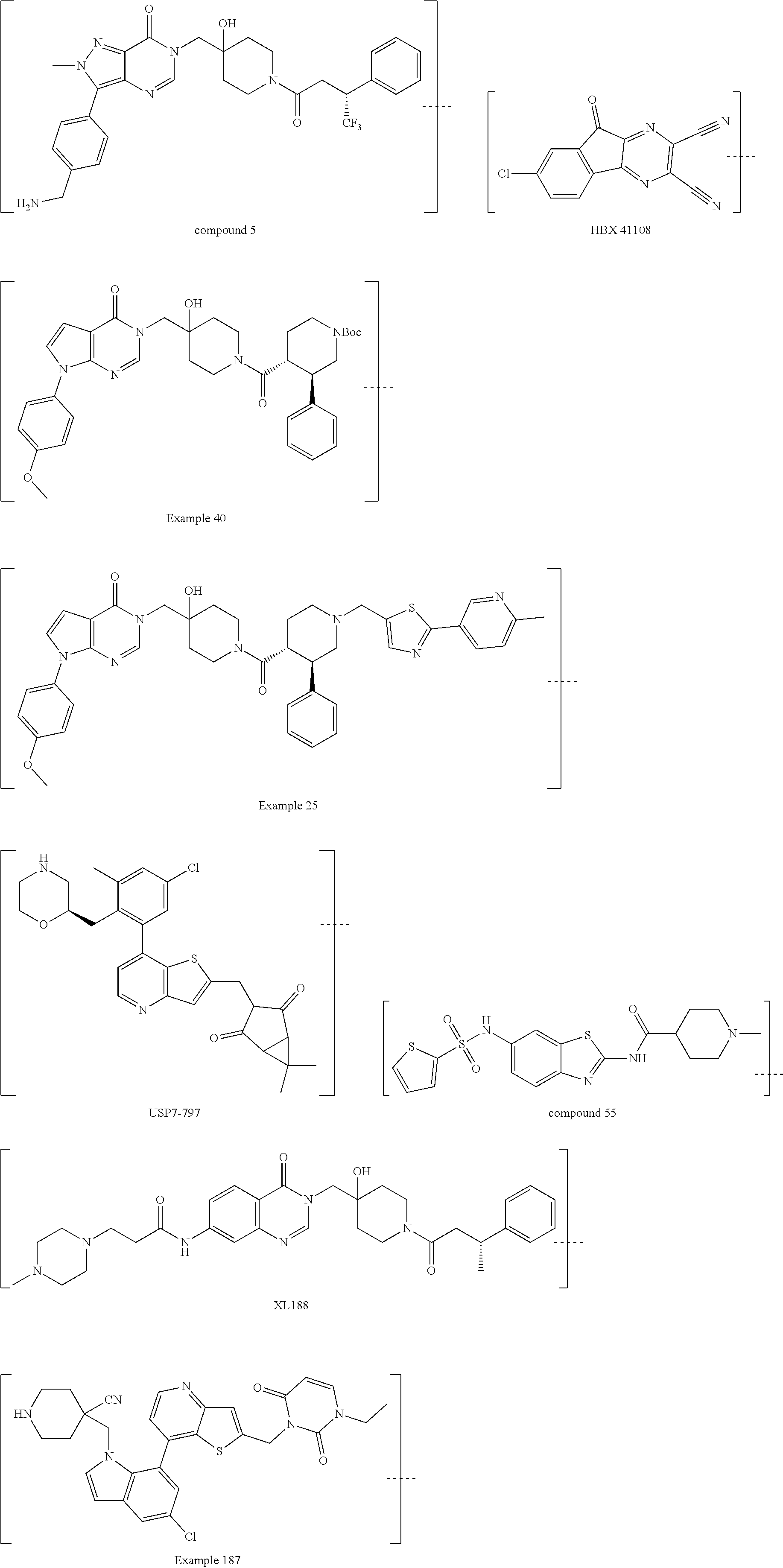
wherein, [symbol] denotes the point of attachment to the linker.

10. The bivalent compound according to any one of claims 1-9, wherein the linker is a moiety of Formula (L): 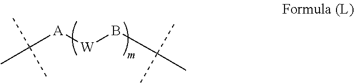
wherein
A, W and B, at each occurrence, are independently selected from null, or bivalent moiety selected from R′—R″, R′COR″, R′CO₂R″, R′C(O)N(R¹)R″, R′C(S)N(R¹)R″, R′OR″, R′OC(O)R″, R′OC(O)OR″, R′OCON(R¹)R″, R′SR″, R′SOR″, R′SO₂R″, R′SO₂N(R¹)R″, R′N(R¹)R″, R′NR¹COR″, R′NR¹C(O)OR″, R′NR¹CON(R²)R″, R′NR¹C(S)R″, R′NR¹S(O)R″, R′NR¹S(O)₂R″, and R′NR¹S(O)₂N(R²)R″, wherein
R′ and R″ are independently selected from a bond, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R^{r} is selected from optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclic, optionally substituted aryl, and optionally substituted heteroaryl;
R′ and R″, R¹ and R², R′ and R¹, R′ and R², R″ and R¹, R″ and R² together with the atom to which they are connected form a 3-20 membered cycloalkyl or 4-20 membered heterocyclic ring; and
m is 0 to 15.

11. The bivalent compound according to any one of claim 1-9, wherein the linker is a moiety of Formula (L-1): 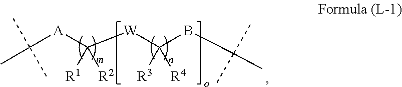
wherein
R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclic, optionally substituted aryl, and optionally substituted heteroaryl, or
R¹ and R², R³ and R⁴ together with the atom to which they are connected form a 3-20 membered cycloalkyl or 4-20 membered heterocyclic ring;
A, W and B, at each occurrence, are independently selected from null, or bivalent moiety selected from R′—R″, R′COR″, R′CO₂R″, R′C(O)N(R⁵)R″, R′C(S)N(R⁵)R″, R′OR″, R′OC(O)R″, R′OC(O)OR″, R′OCONR″R″, R′SR″, R′SOR″, R′SO₂R″, R′SO₂N(R⁵)R″, R′N(R⁵)R″, R′NR⁵COR″, R′NR⁵C(O)OR″, R′NR⁵CON(R⁶)R″, R′NR⁵C(S)R″, R′NR⁵S(O)R″, R′NR⁵S(O)₂R″, and R′NR⁵S(O)₂N(R⁶)R″, wherein
R′ and R″ are independently selected from null, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R^{r} is selected from optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclic, optionally substituted aryl, and optionally substituted heteroaryl;
R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclic ring;
m is 0 to 15;
n, at each occurrence, is 0 to 15; and
o is 0 to 15.

12. The bivalent compound according to any one of claim 1-9, wherein the linker is a moiety of Formula (L-2): 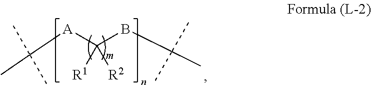
wherein
R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclic, optionally substituted aryl, and optionally substituted heteroaryl, or
R¹ and R² together with the atom to which they are connected form a 3-20 membered cycloalkyl or 4-20 membered heterocyclic ring;
A and B, at each occurrence, are independently selected from null, or bivalent moiety selected from R′—R″, R′COR″, R′CO₂R″, R′C(O)NR³R″, R′C(S)NR³R″, R′OR″, R′OC(O)R″, R′OC(O)OR″, R′OCON(R³)R″, R′SR″, R′SOR″, R′SO₂R″, R′SO₂N(R³)R″, R′N(R³)R″, R′NR³COR″, R′NR³C(O)OR″, R′NR³CON(R⁴)R′, R′NR³C(S)R″, R′NR³S(O)R″, R′NR³S(O)₂R″, and R′NR³S(O)₂N(R⁴)R″, wherein
R′ and R″ are independently selected from null, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R^{r} is selected from optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclic, optionally substituted aryl, and optionally substituted heteroaryl;
R′ and R″, R³ and R⁴, R′ and R³, R′ and R⁴, R″ and R³, R″ and R⁴ together with the atom to which they are connected form a 3-20 membered cycloalkyl or 4-20 membered heterocyclic ring;
m, at each occurrence, is 0 to 15; and
n is 0 to 15.

13. The bivalent compound according to any one of claims 1-9, wherein the linker is a moiety of Formula (L-2): Formula (L-3): 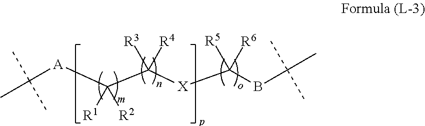
wherein
X is selected from O, NH, and NR⁷;
R¹, R², R³, R₄, R⁵, and R⁶, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered carbocyclyl, optionally substituted 3-8 membered cycloalkoxy, optionally substituted 4-10 membered heterocyclic, optionally substituted aryl, and optionally substituted heteroaryl;
A and B are independently selected from null, or bivalent moiety selected from R′—R″, R′COR″, R′CO₂R″, R′C(O)N(R⁸)R″, R′C(S)N(R⁸)R″, R′OR″, R′OC(O)R″, R′OC(O)OR″, R′OCON(R⁸)R″, R′SR″, R′SOR″, R′SO₂R″, R′SO₂N(R⁸)R″, R′N(R⁸)R″, R′NR⁸COR″, R′NR⁵C(O)OR″, R′NR⁸CON(R⁹)R″, R′NR⁵C(S)R″, R′NR⁸S(O)R″, R′NR⁸S(O)₂R″, and R′NR⁸S(O)₂N(R⁹)R″, wherein
R′ and R″ are independently selected from null, optionally substituted R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R^{r} is selected from optionally substituted 3-10 membered carbocyclyl, optionally substituted 4-10 membered heterocyclic, optionally substituted C₃-C₁₃ fused cycloalkyl, optionally substituted C₃-C₁₃ fused heterocyclic, optionally substituted C₃-C₁₃ bridged cycloalkyl, optionally substituted C₃-C₁₃ bridged heterocyclic, optionally substituted C₃-C₁₃ spiro cycloalkyl, optionally substituted C₃-C₁₃ spiro heterocyclic, optionally substituted aryl, and optionally substituted heteroaryl;
R⁷, R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclic, optionally substituted aryl, and optionally substituted heteroaryl;
R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclic ring;
m, at each occurrence, is 0 to 15;
n, at each occurrence, is 0 to 15;
o is 0 to 15; and
p is 0 to 15.

14. A bivalent compound according to any one of claims 1-9, wherein the linker is selected from null, optionally substituted —CO—(CH₂)₀₋₁₀—, optionally substituted —(CH₂)₀₋₁₀—, optionally substituted —(CH₂)₁₋₂—(CO)NH—(CH₂)₀₋₁₀—, optionally substituted —(CH₂)₁₋₂—(CO)NH—(CH₂)₁₋₃—(OCH₂CH₂)₁₋₇—, optionally substituted —(CH₂)₀₋₁—CO—(CH₂)₁₋₃—(OCH₂CH₂)₁₋₇—, optionally substituted —CO—(CH₂)₀₋₃-(alkenylene)-(CH₂)₀₋₃—, optionally substituted —CO—(CH₂)₀₋₃-(alkynylene)-(CH₂)₀₋₃—, optionally substituted —CO—(CH₂)₀₋₃-(3-8 membered carbocyclyl)-(CH₂)₀₋₃—, optionally substituted —CO—(CH₂)₀₋₃-(3-8 membered heterocyclic)-(CH₂)₀₋₃—, optionally substituted —(CH₂)₀₋₃-(alkenylene)-(CH₂)₀₋₃—, optionally substituted —(CH₂)₀₋₃-(alkynylene)-(CH₂)₀₋₃—, optionally substituted —(CH₂)₀₋₃-(3-8 membered carbocyclyl)-(CH₂)₀₋₃—, optionally substituted —(CH₂)₀₋₃-(3-8 membered heterocyclic)-(CH₂)₀₋₃—, optionally substituted —(CH₂)₀₋₈—R^{r}—(CH₂)₀₋₈—, optionally substituted —(CH₂)₀₋₈—R^{r}—(CO)—(CH₂)₁₋₈—, optionally substituted —(CH₂)₀₋₈—R^{r}—(CH₂)₁₋₂—(CO)—NH—(CH₂)₂₋₉—, optionally substituted —(CH₂)₀₋₈—R^{r}—(CH₂)₁₋₂—(CO)—NH—(CH₂)₁₋₃—(OCH₂CH₂)₁₋₇—, optionally substituted —(CH₂)₀₋₈—R^{r}—(CH₂)₀₋₁—(CO)—(CH₂)₁₋₃—(OCH₂CH₂)₁₋₇—, optionally substituted —(CH₂)₀₋₈—R′—(CO)—(CH₂)₀₋₃-(alkenylene)-(CH₂)₀₋₃—, optionally substituted —(CH₂)₀₋₈—R^{r}—(CO)—(CH₂)₀₋₃-(alkynylene)-(CH₂)₀₋₃—, optionally substituted —(CH₂)₀₋₈—R^{r}—(CO)—(CH₂)₀₋₃-(3-8 membered carbocyclyl)-(CH₂)₀₋₃—, optionally substituted —(CH₂)₀₋₈—R^{r}—(CO)—(CH₂)₀₋₃-(3-8 heterocyclic)-(CH₂)₀₋₃—, optionally substituted —(CH₂)₀₋₈—R^{r}—(CH₂)₀₋₃-(alkenylene)-(CH₂)₀₋₃—, optionally substituted —(CH₂)₀₋₈—R^{r}—(CH₂)₀₋₃-(alkynylene)-(CH₂)₀₋₃—, optionally substituted —(CH₂)₀₋₈—R^{r}—(CH₂)₀₋₃-(3-8 membered carbocyclyl)-(CH₂)₀₋₃—, and optionally substituted —(CH₂)₀₋₈—R^{r}—(CH₂)₀₋₃-(heterocyclic)-(CH₂)₀₋₃—.

15. A bivalent compound according to any one of claims 1-9, wherein the linker is a moiety of Formula (L-2-a) or (L-3-a): 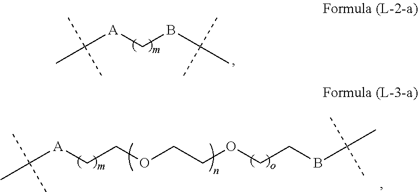
wherein
A is C═O or CH₂,
B is C═O or CH₂, and
m is 0-16,
n is 0-6, and
o is 0-6.

16. A bivalent compound according to any one of claims 1-9, wherein the linker is a moiety of Formula (L-2-b) or (L-3-b): 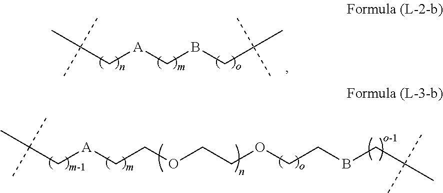
wherein,
A is —C(O)N—,
B is —C(O)N—,
m is 0-16,
n is 0-6,
o is 0-6,
n−1 is 0-6, and
o−1 is 0-6.

17. A bivalent compound according to any one of claims 1-16, wherein the ligand is a moiety of 5′ adenosine monophosphate-activated protein kinase (AMPK) having the structure according to Formula (B-I): 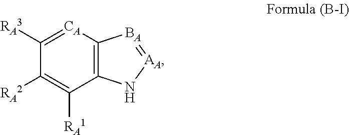
wherein
A_{A} is selected from N, CR_{A}⁴, or 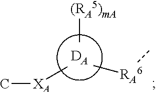
B_{A} is selected from N, CR_{A}⁴, or 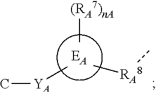 [symbol]
denotes the point of attachment to the linker,
C_{A} is selected from N or CR_{A}⁴;
Ring D_{A} and Ring E_{A} are independently selected from null, C₃-C₁₂ cycloalkyl, 3-12-membered heterocyclic, C₆-C₁₀ aryl, and 5-10 membered heteroaryl;
X_{A} and Y_{A} are independently selected from a bond, —O—, —S—, —NR_{A}⁹—, —C(O)—, —C(O)O—, —C(O)NR_{A}⁹—, —O—C(O)NR_{A}⁹—, —NR_{A}¹⁰C(O)NR_{A}⁹—, —S(O)—, —S(O)₂—, —S(O)NR_{A}⁹—, —S(O)₂NR_{A}⁹—, C₁-C₆ alkylene, C₁-C₆ haloalkylene, C₁-C₆ heteroalkylene, C₂-C₆ alkenylene, and C₂-C₆ alkynylene;
R_{A}¹, R_{A}², each R_{A}⁴, each R_{A}⁵, and each R_{A}⁷ are independently selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic;
R_{A}³ is selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl; or
R_{A}¹ and R_{A}², R_{A}² and R_{A}³, R_{A}³ and R_{A}⁴, two R_{A}⁵, or two R_{A}⁷, together with the atoms to which they are attached, optionally form C₄-C₆ cycloalkyl or 5-7 membered heterocyclic, C₆-C₁₀ aryl, and 5-10 membered heteroaryl;
R_{A}⁶ and R_{A}⁸ are independently selected from a bond, —O—, —NR_{A}¹³—, —C(O)—, —C(O)O—, —C(O)NR_{A}¹³—, —O—C(O)NR_{A}¹³—, —NR_{A}¹⁴C(O)NR_{A}¹³—, —S(O)—, —S(O)₂—, —S(O)NR_{A}¹³—, —S(O)₂NR_{A}¹³—, C₁-C₆ alkylene, C₁-C₆ haloalkylene, C₁-C₆ heteroalkylene, C₂-C₆ alkenylene, C₂-C₆ alkynylene, C₆-C₁₀ arylene, and 5-10 membered heteroarylene;
each R_{A}⁹, each R_{A}¹⁰, each R_{A}¹¹, each R_{A}¹², each R_{A}¹³, and each R_{A}¹⁴ are independently selected from H, C₁-C₆ alkyl, C₁-C₆ haloalkyl, C₁-C₆ heteroalkyl, C₃-C₁₂ cycloalkyl, and 3-12 membered heterocyclic, C₆-C₁₀ aryl, and 5-10 membered heteroaryl; or
R_{A}¹¹ and R_{A}¹² together with the atoms to which they are attached, optionally form 3-12 membered heterocyclic; and
m_{A} and n_{A} are independently selected from 0, 1, 2, 3, and 4.

18. The bivalent compound according to claim 17, wherein the AMPK ligand is selected from a structure according to Formulae (B-I-a2), (B-I-a3), (B-I-a4), (B-I-b3), or (B-I-b4): 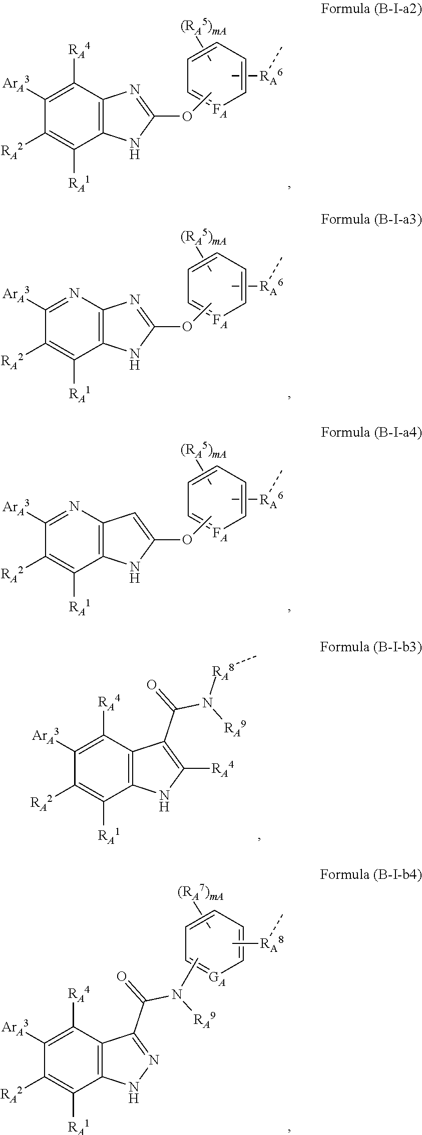
wherein
F_{A} is selected from N or CR_{A}⁵; and
G_{A} is selected from N or CR_{A}⁷.

19. The bivalent compound according to claim 17, wherein the AMPK ligand is selected from a structure according to Formulae (B-I-a5), (B-I-a6), (B-I-a7), (B-I-b5) or (B-I-b6): 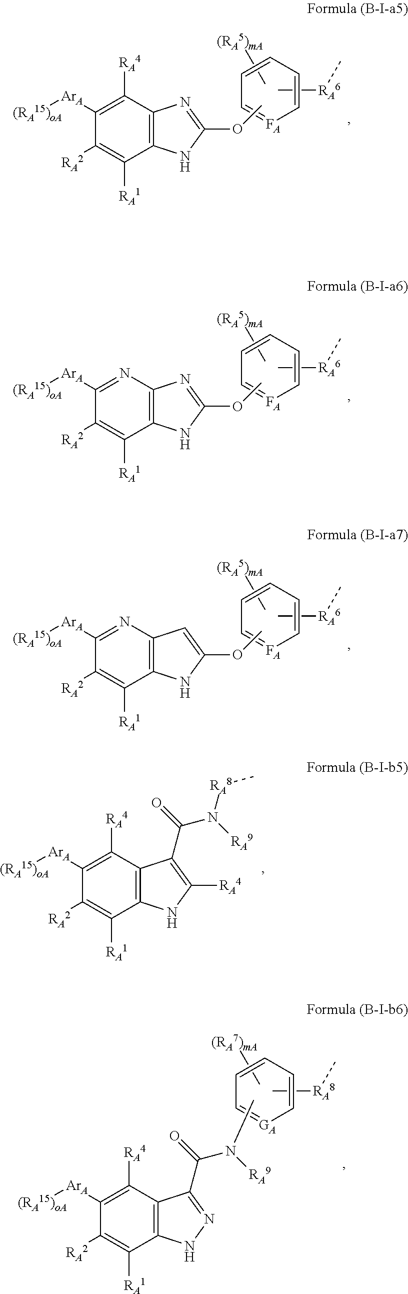
wherein Ar_{A} is selected from C₆-C₁₀ aryl, or 5-10 membered heteroaryl;
each R_{A}¹⁵ is independently selected from H, halogen, cyano, OR_{A}¹¹, NR_{A}¹¹R_{A}¹², C(O)OR_{A}¹¹, C(O)NR_{A}¹¹R_{A}¹², S(O)₂NR_{A}¹¹R_{A}¹², —N═S(O)R_{A}¹¹R_{A}¹², C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl and 5-10 membered heteroaryl, or
two of R_{A}¹⁵, together with the atoms to which they are attached, optionally form C₃-C₁₂ cycloalkyl, 3-12 membered heterocyclic, C₆-C₁₀ aryl or 5-10 membered heteroaryl; and
o_{A} is selected from 0, 1, 2, 3, 4, 5, or 6.

20. A bivalent compound according to any one of claims 1-16, wherein the ligand is selected from a derivative of one of the following compounds:

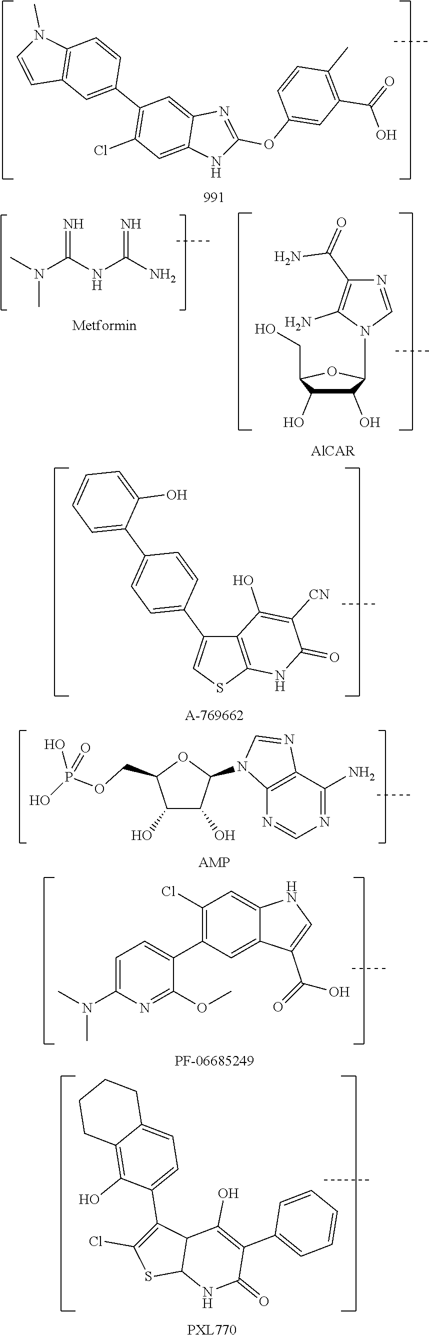

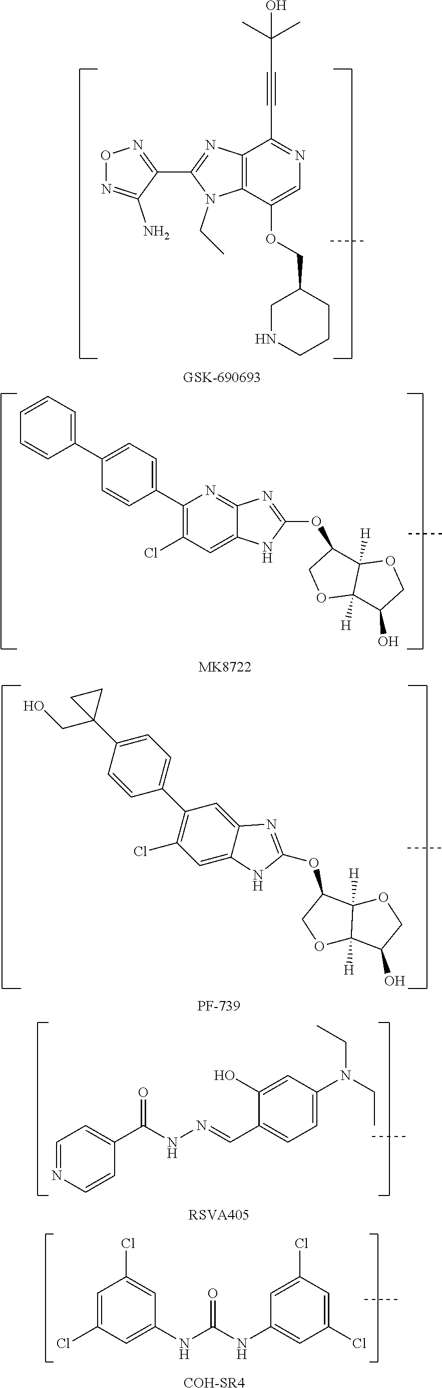

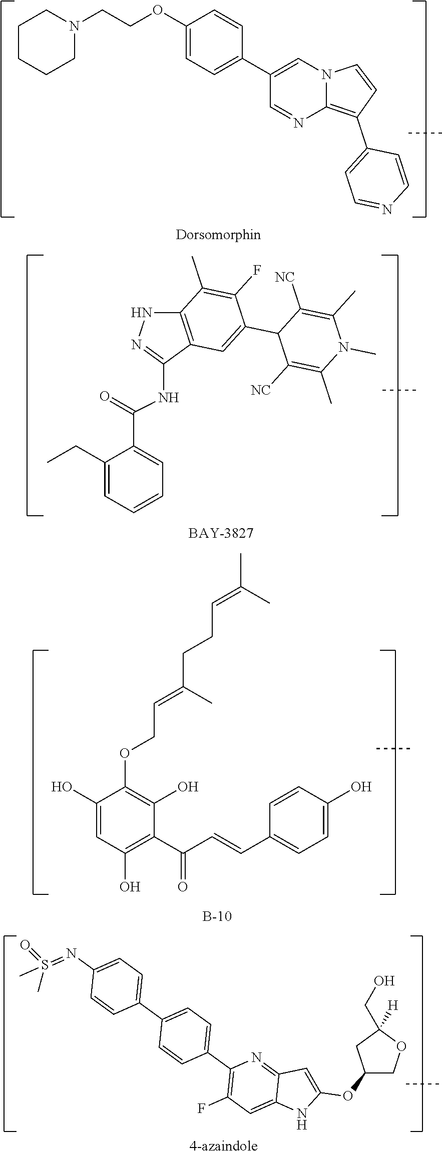

21. A bivalent compound according to any one of claims 1-16, wherein the ligand is a moiety of cyclic GMP-AMP (cGAMP) synthase (cGAS) having the structure according to Formula (C—I): 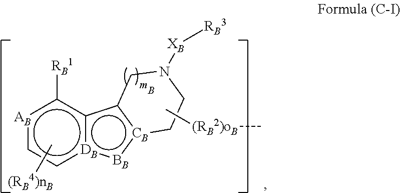
wherein [symbol]
denotes the point of attachment to the linker; [symbol]
connects to either R_{B}^{1b} or R_{B}^{3b}, with the proviso that when [symbol] connects to R_{B}^{1b}, R_{B}³ is R_{B}^{3a}; and when [symbol] connects to R_{B}^{3b}, R_{B}¹ is R_{B}^{1a};
A_{B} is selected from N and CR_{B}⁴;
B_{B} is selected from N, O, S, CR_{B}⁵ and NR_{B}⁵;
C_{B} is selected from N and C;
D_{B} is selected from N and C;
X_{B} is X_{B}^{a}—X_{B}^{b};
X_{B}^{a} is selected from a bond, —C(O)—, —C(O)O—, —C(O)NR_{B}⁶—, —S(O)—, —S(O)₂—, —S(O)NR_{B}⁶—, —S(O)₂NR_{B}⁶—;
X_{B}^{b} is selected from a bond, C₁-C₆ alkylene, C₁-C₆ haloalkylene, C₁-C₆ heteroalkylene, C₃-C₁₂ cycloalkylene, and 3-12 membered heterocyclicene, C₆-C₁₀ arylene, and 5-10 membered heteroarylene;
R_{B}¹ is selected from monovalent group R_{B}^{1a} and bivalent group R_{B}^{1b};
R_{B}^{1a} is selected from H, halogen, cyano, OR_{B}⁷, NR_{B}⁷R_{B}⁸, C(O)OR_{B}⁷, C(O)NR_{B}⁷R_{B}⁸, S(O)₂NR_{B}⁷R_{B}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl;
R_{B}^{1b} is connected to the linker, and is selected from a bond, —O—, —NR_{B}⁷—, —C(O)O—, —C(O)NR_{B}⁷—, —S(O)₂NR_{B}⁷—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, 3-12 membered heterocyclicene, C₆-C₁₀ arylene, and 5-10 membered heteroarylene;
each R_{B}² is independently selected from H, halogen, C₁-C₆ alkyl, C₁-C₆ haloalkyl, C₁-C₆ heteroalkyl, C₃-C₁₂ cycloalkyl, or
two R_{B}², together with the atoms to which they are attached, optionally form C₃-C₁₂ cycloalkyl or 3-12 membered heterocyclic;
R_{B}³ is selected from monovalent group R_{B}^{3a} and bivalent group R_{B}^{3b};
R_{B}^{3a} is selected from H, halogen, OR_{B}⁹, NR_{B}⁹R_{B}¹⁰, C(O)OR_{B}⁹, C(O)NR_{B}⁹R_{B}¹⁰, S(O)₂NR_{B}⁹R_{B}¹⁰, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic;
R_{B}^{3b} is connected to the linker, and is selected from a bond, —O—, —NR_{B}⁹—, —C(O)O—, —C(O)NR_{B}⁹—, —S(O)₂NR_{B}⁹—, C₁-C₆ alkylene, C₁-C₆ haloalkylene, C₁-C₆ heteroalkylene, C₂-C₆ alkenylene, C₂-C₆ alkynylene, C₃-C₁₂ cycloalkylene, and 3-12 membered heterocyclicene;
each R_{B}⁴ is independently selected from H, halogen, cyano, OR_{B}⁷, NR_{B}⁹R_{B}⁸, C(O)OR_{B}⁷, C(O)NR_{B}⁷R_{B}⁸, S(O)₂NR_{B}⁷R_{B}⁸, C₁-C₃ alkyl, C₁-C₃ haloalkyl, C₁-C₃ heteroalkyl, C₂-C₄ alkenyl, C₂-C₄ alkynyl, C₃-C₆ cycloalkyl, and 3-6 membered heterocyclic, or
two R_{B}⁴, together with the atoms to which they are attached, optionally form partially unsaturated C₃-C₁₂ cycloalkyl, partially unsaturated 3-12 membered heterocyclic, C₆-C₁₀ arylene, or 5-10 membered heteroarylene;
R_{B}⁵ is selected from H, halogen, C₁-C₃ alkyl, C₁-C₃ haloalkyl, C₁-C₃ heteroalkyl, C₃-C₆ cycloalkyl, and 3-6 membered heterocyclic;
R_{B}⁶, each R_{B}⁷, each R_{B}⁸, each R_{B}⁹, and each R_{B}¹⁰, are independently selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic, or
R_{B}⁷ and R_{B}⁸, and R_{B}⁹ and R_{B}¹⁰ together with the atoms to which they are attached, optionally form 3-12 membered heterocyclic;
m_{B} is selected from 1 and 2;
n_{B} is selected from 0, 1, 2 and 3; and
o_{B} is selected from 0, 1, 2, 3, 4, 5 and 6.

22. A bivalent compound according to claim 21, wherein the ligand is a moiety having the structure according to Formulae (C—I-a2) or (C—I-b2): 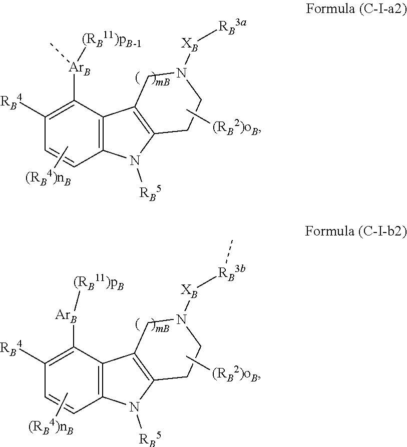
wherein
Ar_{B} is selected from C₆-C₁₀ aryl, and 5-10 membered heteroaryl;
each R_{B}¹¹ is independently selected from H, halogen, cyano, OR_{B}⁷, NR_{B}⁷R_{B}⁸, C(O)OR_{B}⁷, C(O)NR_{B}⁷R_{B}⁸, S(O)₂NR_{B}⁷R_{B}⁸, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, and 5-10 membered heteroaryl; or
two R_{B}¹¹, together with the atoms to which they are attached, optionally form partially unsaturated C₃-C₁₂ cycloalkyl, partially unsaturated 3-12 membered heterocyclic, C₆-C₁₀ aryl, or 5-10 membered heteroaryl;
p_{B} is selected from 0, 1, 2, 3, 4, and 5; and
p_{B-1} is selected from 0, 1, 2, 3, and 4.

23. A bivalent compound according to any one of claims 1-16, wherein the ligand is selected from a derivative of one of the following compounds:

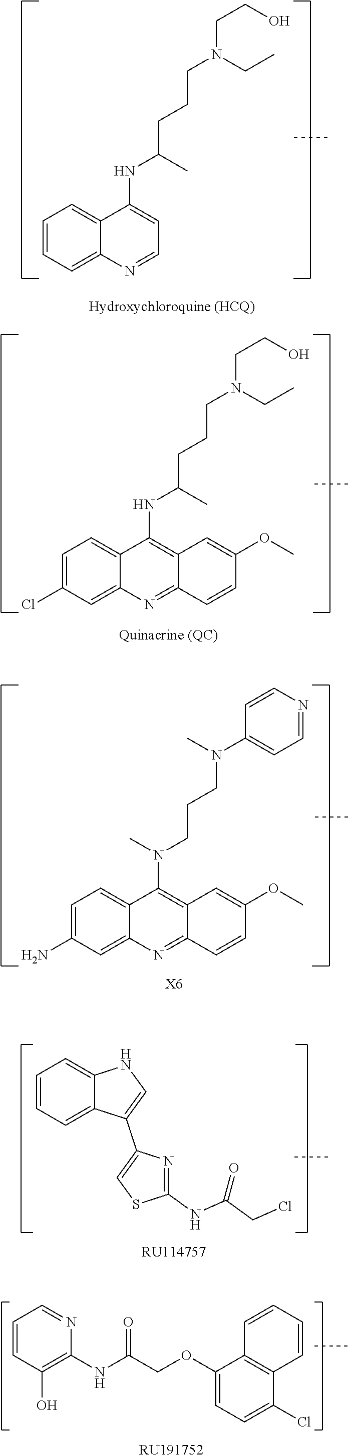

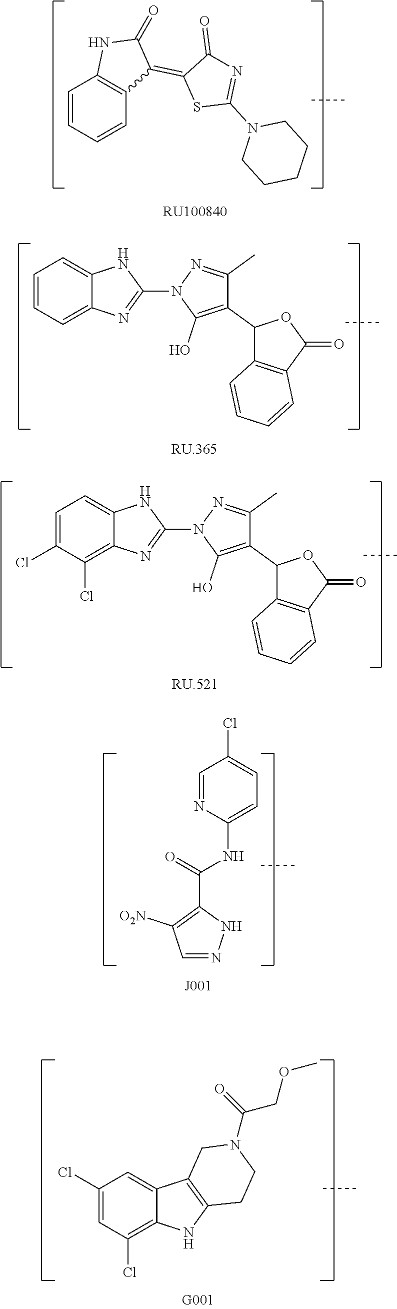

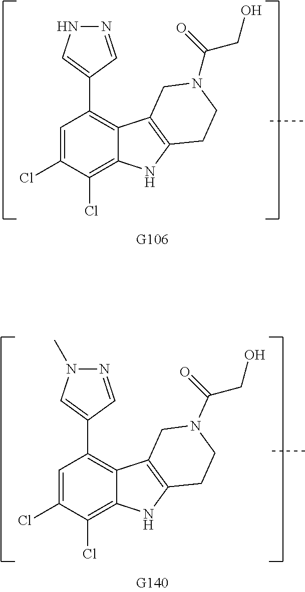

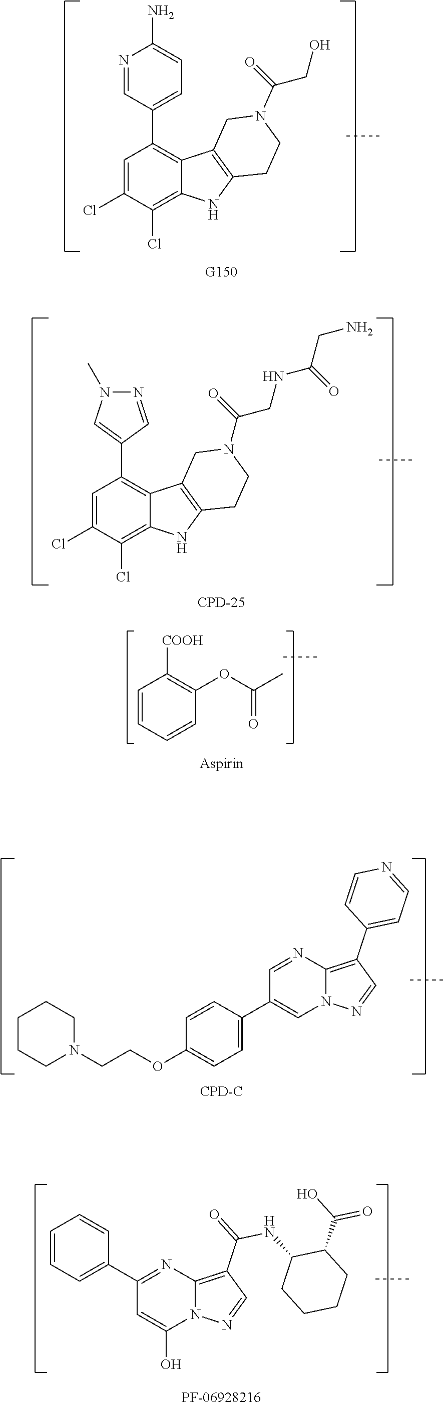

24. A bivalent compound according to any one of claims 1-16, wherein the ligand is a moiety of cystic fibrosis transmembrane conductance regulator (CFTR) having the structure according to Formula (D-I): 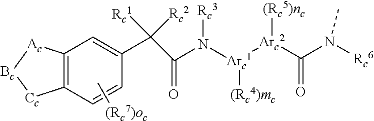
wherein [symbol]
denotes the point of attachment to the linker,
A_{C} and C_{C} are independently selected from O, S, or C(R_{C}⁸)(R_{C}⁹);
B_{C} is C(R_{C}¹⁰)(R_{C}¹¹) or NR_{C}¹²;
Ar_{C}¹ and Ar_{C}² are independently selected from null, C₆-C₁₀ aryl and 5-10 membered heteroaryl;
R_{C}¹ and R_{C}² are independently selected from H, halo, OR_{C}¹³, NR_{C}¹³R_{C}¹⁴, C(O)OR_{C}¹³, C(O)NR_{C}¹³R_{C}¹⁴, S(O)₂NR_{C}¹³R_{C}¹⁴, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic, or
R_{C}¹ and R_{C}², together with the atoms to which they are attached, optionally form C₃-C₁₂ cycloalkyl;
R_{C}³ and R_{C}⁶ are independently selected from H, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic;
each R_{C}⁴, each R_{C}⁵, and each R_{C}⁷ are independently selected from H, halogen, cyano, OR_{C}¹³, NR_{C}¹³R_{C}¹⁴, C(O)OR_{C}¹³, C(O)NR_{C}¹³R_{C}¹⁴, S(O)₂NR_{C}¹³R_{C}¹⁴, C₁-C₆ alkyl, C₁-C₆ haloalkyl, C₁-C₆ heteroalkyl, C₂-C₆ alkenyl, C₂-C₆ alkynyl, C₃-C₁₂ cycloalkyl, and 3-12 membered heterocyclic,
two R_{C}⁴, two R_{C}⁵, or two R_{C}⁷, together with the atoms to which they are attached, optionally form partially unsaturated C₃-C₁₂ cycloalkyl, partially unsaturated 3-12 membered heterocyclic, C₆-C₁₀ aryl and 5-10 membered heteroaryl;
R_{C}⁸, R_{C}⁹, R_{C}¹⁰ and R_{C}¹¹ are independently selected from H, halogen, OR_{C}¹³, NR_{C}¹³R_{C}¹⁴, C(O)OR_{C}¹³, C(O)NR_{C}¹³R_{C}¹⁴, S(O)₂NR_{C}¹³R_{C}¹⁴, C₁-C₆ alkyl, C₁-C₆ haloalkyl, C₁-C₆ heteroalkyl, C₃-C₁₂ cycloalkyl, and 3-12 membered heterocyclic, or
R_{C}⁸ and R_{C}⁹, and R_{C}¹⁰ and R_{C}¹¹, together with the atoms to which they are attached, optionally form C₃-C₁₂ cycloalkyl and 3-12 membered heterocyclic;
R_{C}¹² is selected from H, C₁-C₆ alkyl, C₁-C₆ haloalkyl, C₁-C₆ heteroalkyl, C₃-C₁₂ cycloalkyl, and 3-12 membered heterocyclic;
R_{C}¹³ and R_{C}¹⁴ are independently selected from H, C₁-C₆ alkyl, C₁-C₆ haloalkyl, C₁-C₆ heteroalkyl, C₃-C₁₂ cycloalkyl, and 3-12 membered heterocyclic, or R_{C}¹³ and R_{C}¹⁴ together with the atoms to which they are attached, optionally form 3-12 membered heterocyclic;
m_{C} is 0, 1, 2, or 3;
n_{C} is 0, 1, 2, or 3; and
o_{C} is 0, 1, 2, or 3.

25. A bivalent compound according to any one of claims 1-16, wherein the ligand is selected from a derivative of one of the following compounds:

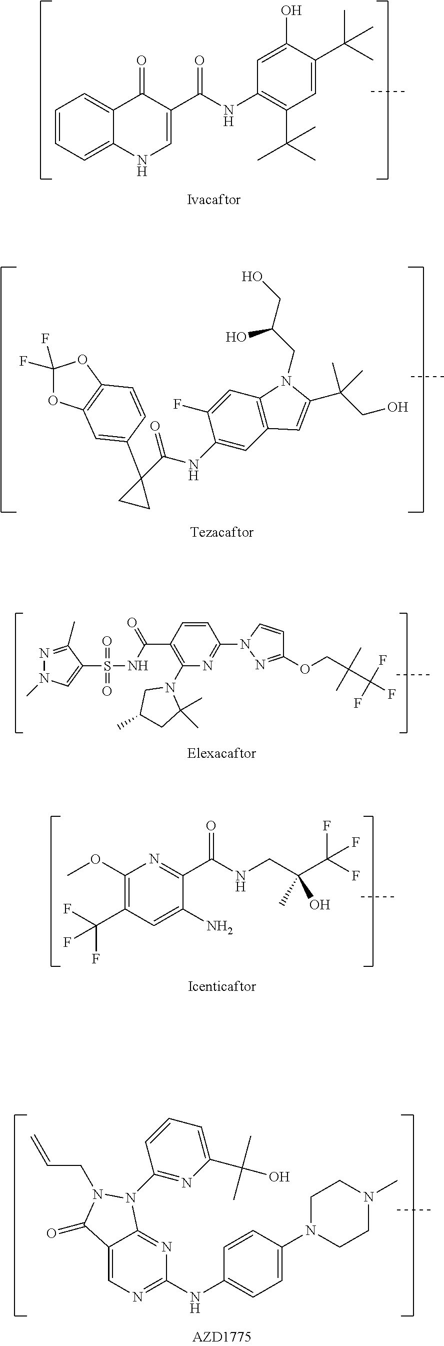

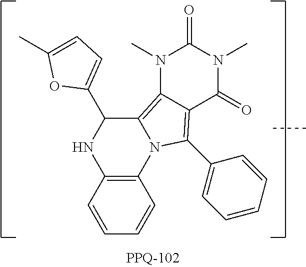

26. A bivalent compound selected from the group consisting of:
XH181-1, XH181-2, XH181-3, XH181-4, XH181-5, XH181-6, XH181-7, XH181-8, XH181-9, XH181-15, XH188-16, XH188-17, XH188-18, XH188-19, KL186-1, KL186-2, KL186-3, KL186-4, KL186-5, KL186-6, KL186-7, KL186-8, KL186-9, KL186-10, KL186-11, KL186-12, KL186-13, KL186-14, KL186-48, KL186-49, KL186-50, KL186-51, KL186-52, KL186-53, KL186-54, KL186-55, KL186-56, KL186-57, KL186-58, KL186-59 and KL186-60.

27. A bivalent compound selected from the group consisting of:
XH168-142, XH168-143, XH168-144, XH168-145, XH168-146, XH168-147, XH168-148, XH168-149, XH168-150, XH168-151, XH168-152, XH168-153, XH168-154, XH168-155, XH181-72, XH181-73, XH181-90, XH181-91, XH181-92, XH181-93, XH181-94, XH181-95, XH181-96, XH181-97, XH181-99, XH181-100, XH181-101, XH181-102, XH181-103, XH188-48, XH188-49, XH188-50, XH188-51, Xh188-52, XH188-53, XH188-54, XH188-55, XH188-56, XH188-57, XH188-58, XH188-59, XH188-60, XH188-90, XH188-91, XH188-92, XH188-93, XH188-94, XH188-95, XH188-96, XH188-97, XH188-98, XH188-99, XH188-100, XH188-101, XH188-102, KL186-107, KL186-115, KL186-116, KL186-117, KL186-118, KL186-119, KL186-120, KL186-121, KL186-122, KL186-123, KL186-124, KL186-125, KL186-126, KL186-127, KL186-128, KL186-129, KL186-130, KL186-131, KL186-132, KL186-133, KL186-134, KL186-135, KL186-136, KL186-137, KL186-138, KL186-139, KL186-140, KL186-141, KL186-142, KL186-143, KL186-144, KL186-145, KL186-146, KL186-147, KL186-148, KL186-149, v50, KL186-151, KL186-152, KL186-153, KL186-154, KL186-155, KL186-156, KL186-157, KL186-158, KL186-159, KL186-160, KL186-161, KL186-162, KL186-163, KL186-164, KL186-165, KL186-166, KL186-167, KL186-168, KL186-169, KL186-170 and KL186-171.

28. A bivalent compound selected from the group consisting of:
XH168-51, XH168-32, XH168-33, XH168-59, XH168-66, XH168-67, XH168-68, XH168-69, XH168-70, XH168-58, XH168-47, XH168-48, XH168-49, XH168-50, KL180-124, KL180-125, KL180-126, KL180-127, KL180-128, KL180-129, KL180-130, KL180-131, KL180-132, KL180-133, KL180-134, KL180-135, KL180-136, KL180-137, KL180-162, KL180-163, KL180-164, KL180-165, KL180-166, KL180-167, KL180-168, KL180-169, KL180-170, KL180-171, KL180-172, KL180-173 and KL180-174.

29. The bivalent compound (R)-5-((6-chloro-5-(1-methyl-1H-indol-5-yl)-1H-benzo[d]imidazol-2-yl)oxy)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-4-oxo-8,11,14,17-tetraoxa-2,5-diazanonadecan-19-yl)-2-methylbenzamide (XH181-18, MS8118).

30. The bivalent compound 6′-amino-N-(2-(9-(5-((6-chloro-5-(1-methyl-1H-indol-5-yl)-1H-benzo[d]imidazol-2-yl)oxy)-2-methylbenzamido)nonanamido)ethyl)-4′-ethyl-5′-(4-hydroxyphenyl)-[3,3′-bipyridine]-6-carboxamide (KL186-55, MS8655).

31. The bivalent compound 6′-amino-N-(2-(3-(2-(5-((6-chloro-5-(1-methyl-1H-indol-5-yl)-1H-benzo[d]imidazol-2-yl)oxy)-2-methylbenzamido)ethoxy)propanamido)ethyl)-4′-ethyl-5′-(4-hydroxyphenyl)-[3,3′-bipyridine]-6-carboxamide (KL186-56, MS8656).

32. The bivalent compound 6′-amino-N-(1-(5-((6-chloro-5-(1-methyl-1H-indol-5-yl)-1H-benzo[d]imidazol-2-yl)oxy)-2-methylphenyl)-1,11-dioxo-5,8-dioxa-2,12-diazatetradecan-14-yl)-4′-ethyl-5′-(4-hydroxyphenyl)-[3,3′-bipyridine]-6-carboxamide (KL186-57, MS8657).

33. The bivalent compound (R)-3-(6-(1-(2,2-difluorobenzo[d][1,3]dioxol-5-yl)cyclopropane-1-carboxamido)-3-methylpyridin-2-yl)-N-(9-(2-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)acetamido)nonyl)benzamide (XH168-69, MS6869).

34. A bivalent compound comprising a ligand bound to a de-ubiquitination tag through a linker, the de-ubiquitination tag including a ubiquitin-specific-processing protease 7 (USP7) recruiter according to Formula (E-I-a): 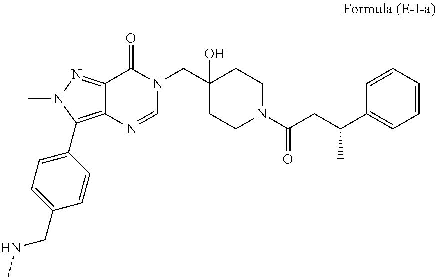
wherein the ligand has the following structure: 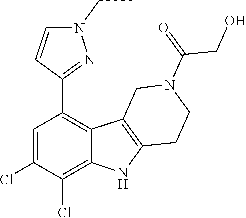
and the linker is selected from one of Formulas L-2-b and L-3-b: 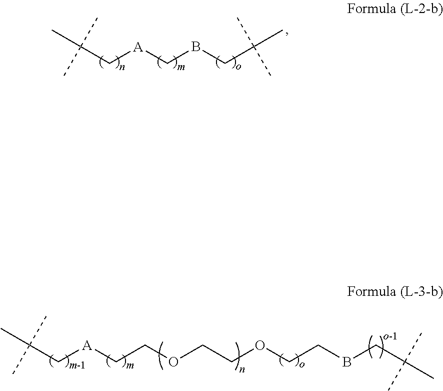
wherein,
A is —C(O)N—, or —NC(O)—
B is —C(O)N—, or —NC(O)—
m is 0-16,
n is 0-6,
o is 0-6,
n−1 is 0-6, and
o−1 is 0-6.

35. A pharmaceutical composition, comprising a compound according to any one of claims 1-34 and a pharmaceutically acceptable carrier.

36. A pharmaceutical composition according to claim 35, wherein the compound is present in an effective amount.

37. A pharmaceutical composition according to claim 35, wherein the composition is formulated for administration by oral, parenteral or transdermal delivery.

38. A method of treating an AMPK-mediated disease, comprising administering to a subject in need thereof a bivalent compound according to any one of claims 1-20 and 26.

39. A method of treating a cGAS-mediated disease, comprising administering to a subject in need thereof a bivalent compound according to any one of claims 1-16, 21-23 and 27.

40. A method of treating a CFTR-mediated disease, comprising administering to a subject in need thereof a bivalent compound according to any one of claims 1-16, 24, 25 and 28.

41. The method according to claim 38, wherein the disease is selected from cancer, metabolic syndrome, type-2 diabetes, non-alcoholic fatty liver disease and cardiovascular disease.

42. The method according to claim 38, wherein the AMPK-mediated cancer is selected from mesothelioma, hepatocellular cancer, central nervous system neoplasm, lung cancer, bone cancer, pancreatic cancer, skin cancer, head and neck cancer, melanoma, ovarian cancer, colon cancer, rectal cancer, anal cancer, stomach cancer, gastrointestinal cancer, breast cancer (e.g., estrogen receptor positive (ER+) breast cancer), uterine cancer, fallopian tube cancer, endometrial cancer, cervical cancer, vaginal cancer, vulvar cancer, esophageal cancer, gastrointestinal cancer, endocrine cancer, thyroid cancer, parathyroid cancer, adrenal cancer, soft tissue sarcoma, urethral cancer, penile cancer, prostate cancer, testicular cancer, leukemia, lymphoma, bladder cancer, renal cell cancer, brain stem glioma, pituitary cancer, adrenocortical cancer, gallbladder cancer, multiple myeloma, cholangiocarcinoma, fibrosarcoma, neuroblastoma, and/or retinoblastoma.

43. The method according to claim 39, wherein the disease is selected from mesothelioma, hepatocellular cancer, central nervous system neoplasm, lung cancer, bone cancer, pancreatic cancer, skin cancer, head and neck cancer, melanoma, ovarian cancer, colon cancer, rectal cancer, anal cancer, stomach cancer, gastrointestinal cancer, breast cancer (e.g., estrogen receptor positive (ER+) breast cancer), uterine cancer, fallopian tube cancer, endometrial cancer, cervical cancer, vaginal cancer, vulvar cancer, esophageal cancer, gastrointestinal cancer, endocrine cancer, thyroid cancer, parathyroid cancer, adrenal cancer, soft tissue sarcoma, urethral cancer, penile cancer, prostate cancer, testicular cancer, leukemia, lymphoma, bladder cancer, renal cell cancer, brain stem glioma, pituitary cancer, adrenocortical cancer, gallbladder cancer, multiple myeloma, cholangiocarcinoma, fibrosarcoma, neuroblastoma, and/or retinoblastoma.

44. The method according to claim 40, wherein the disease is selected from cystic fibrosis, male infertility, polycystic kidney disease, obstructive lung disease, intestinal obstruction syndromes, liver dysfunction, exocrine and endocrine pancreatic dysfunction and secretory diarrhea.
